(19) United States
(12) Patent Application Publication
(10) Pub. No.: US 2025/0228865 A1
(43) Pub. Date: Jul. 17, 2025

(54) COMPOUNDS FOR ENHANCING READ THROUGH OF GENES CONTAINING PREMATURE TERMINATION CODONS AND METHODS FOR MAKING AND USING THE SAME

(71) Applicant: Sanofi US Services Inc., Bridgewater, NJ (US)

(72) Inventors: Neil Castle, Cary, NC (US); Martin Smrcina, Oro Valley, AZ (US); Sylvain Lebreton, Tucson, AZ (US); Ingrid Attinost, Oro Valley, AZ (US); Anil Nair, Tucson, AZ (US); Andrea Fiumana, Ely (Cambs) (GB); Rachel Parsons, Royston (Herts) (GB); Mathew Calder, Cambridge (Cambs) (GB); Paul Webb, Chatteris (Cambs) (GB); Jared Marklew, Royston (Herts) (GB); Simon Bedford, Harlow (Essex) (GB)

(73) Assignee: Sanofi US Services Inc., Bridgewater, NJ (US)

(21) Appl. No.: 18/960,716

(22) Filed: Nov. 26, 2024

Related U.S. Application Data
(63) Continuation of application No. PCT/US2023/023979, filed on May 31, 2023.
(60) Provisional application No. 63/347,973, filed on Jun. 1, 2022.

(51) Int. Cl. A61K 31/5377; A61K 31/365; A61K 31/40; A61K 31/4015; A61K 31/421; A61K 31/4439; A61K 31/506; A61K 31/5375; A61K 31/5386; A61K 45/06; A61P 11/00; C07D 207/404; C07D 231/12; C07D 233/64; C07D 235/02; C07D 239/47; C07D 249/06; C07D 249/08; C07D 257/04; C07D 263/24; C07D 265/30; C07D 271/06; C07D 307/58; C07D 401/04; C07D 403/04; C07D 403/14; C07D 413/04; C07D 498/04; C07D 498/08

(52) U.S. Cl. CPC A61K 31/5377; A61K 31/365; A61K 31/40; A61K 31/4015; A61K 31/421; A61K 31/4439; A61K 31/506; A61K 31/5375; A61K 31/5386; A61K 45/06; A61P 11/00; C07D 207/404; C07D 231/12; C07D 233/64; C07D 235/02; C07D 239/47; C07D 249/06; C07D 249/08; C07D 257/04; C07D 263/24; C07D 265/30; C07D 271/06; C07D 307/58; C07D 401/04; C07D 403/04; C07D 403/14; C07D 413/04; C07D 498/04; C07D 498/08

(57) ABSTRACT

Disclosed herein are new aromatic compounds, compositions that include one or more aromatic compounds, and methods of synthesizing the same. Also disclosed herein are methods of enhancing readthrough of genes containing premature termination codons with one or more compounds or compositions described herein.

### CROSS-REFERENCE TO RELATED APPLICATIONS

This application is a continuation of International Application No. PCT/US2023/023979, filed May 31, 2023, which claims the benefit of U.S. Provisional Application No. 63/347,973, filed Jun. 1, 2022, the contents of all of which are incorporated herein by reference in their entirety.

### BACKGROUND

### Field

The present application relates to the fields of chemistry, biochemistry and medicine. More particularly, disclosed herein are new compounds, compositions that include one or more compounds, and methods of using and synthesizing the same. Also disclosed herein are methods of enhancing readthrough of genes containing premature termination codons with one or more compounds or compositions described herein.

### Description of the Related Art

Premature termination codons are codon sequences introduced into DNA through single nucleotide mutations that shift a normal triplet codon sequence into one of the three stop codons, TAA, TAG, or TGA, and stop transcription early, resulting in a loss of protein expression or production of truncated proteins with only residual function. By creating a dearth of protein expression, premature termination codons can be responsible for a number of genetic disorders and diseases including cystic fibrosis, amyloidosis, Duchenne muscular dystrophy, and a variety of cancers. However, sometimes the premature stop codons may be bypassed, resulting in the synthesis of full-length proteins. This phenomenon is known as stop codon readthrough.

Diseases and disorders associated with premature stop codons may be amenable to treatments that may allow readthrough of premature stop codons. Aminoglycoside compounds were shown to facilitate readthrough of premature stop codons over three decades ago. See, e.g, Burke J. F., Mogg A. E. Suppression of a nonsense mutation in mammalian cells in vivo by the aminoglycoside antibiotics G-418 and paromomycin. Nucleic Acids Res. 1985; 13:6265-6272. Since this first identification, additional aminoglycoside and non-aminoglycoside compounds were found to have activity in facilitating readthrough, including geneticin, paramomycin, neomycin, 2,6-diaminopurine and others. Ataluren, a non-aminoglycoside, was found to have therapeutic effects in connection with premature termination codon readthrough and has been approved in several countries for use in treating nonsense mutation Duchenne muscular dystrophy patients.

As readthrough modulation of premature stop codons has shown promising results in the treatment of a variety of disorders, a need exists for additional compounds and therapies that can enhance the readthrough of genes that will benefit patients suffering from premature stop codon related disorders.

### SUMMARY

Some embodiments disclosed herein include a compound having the structure of

### Formula (I):

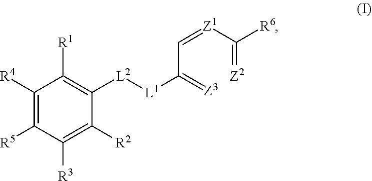

or a pharmaceutically acceptable salt thereof, wherein:
R¹, R², R³, R⁴, and R⁵ are each independently selected from the group consisting of: hydrogen, halo, —CN, —NO₂, —OH, C₁₋₆ alkyl, C₁₋₆ haloalkyl, C₁₋₆ alkoxy, C₁₋₆ haloalkoxy, and C₃₋₈ cycloalkyl;
L¹ is O, NH, or CH₂;
L² is CH₂, CHF, or CF₂;
Z¹ is CH, N, or C—OCH₃;
Z² is CH or N;
Z³ is CH, N, or CF;
R⁶ is optionally substituted heterocyclyl, optionally substituted heteroaryl, or —NR^{7a}R^{7b}; and
R^{7a} and R^{7b} are each independently selected from the group consisting of: hydrogen, optionally substituted C₁₋₆ alkyl, optionally substituted C₃₋₈ cycloalkyl; optionally substituted aryl, optionally substituted heterocyclyl, and optionally substituted heteroaryl.

In some embodiments, when R¹ and R² are both hydrogen and R⁶ is optionally substituted pyrazolyl, then R³ is not halo and R⁴ is not halo.

In some embodiments, L¹ is O. In other embodiments, L¹ is CH₂. In yet other embodiments, L¹ is NH. In some embodiments, L¹ is CHD. In some embodiments, L¹ is CD₂.

In some embodiments, L² is CH₂. In other embodiments, L² is CHF. In yet other embodiments, L² is CF₂. In some embodiments, L² is CHD. In some embodiments, L² is CD₂.

In some embodiments, Z¹ is N, Z² is N, and Z³ is CH. In other embodiments, Z¹ is N, Z² is CH, and Z³ is CH. In yet other embodiments, Z¹ is CH, Z² is CH, and Z³ is CH. In some embodiments, Z¹ is CH, Z² is CH, and Z³ is N. In still yet other embodiments, Z¹ is CH, Z² is CH, and Z³ is CF. In some embodiments, Z¹ is N, Z² is CH, and Z³ is N. In other embodiments, Z¹ is CH, Z² is N, and Z³ is N.

In some embodiments, R¹ is selected from the group consisting of —CN, halo, —C₁₋₆ alkyl, —C₁₋₆ alkoxy, —C₁₋₆ haloalkyl, and —C₁₋₆ haloalkoxy. In some embodiments, R¹ selected from the group consisting of: —CN, —F, —C₁, —Br, —CH₃, —CH₂CH₃, —CF₃, —CF₂CH₃, —OCF₃, —OCHF₂, and -cyclopropyl.

In some embodiments, R² is selected from the group consisting of —CN, halo, —C₁₋₆ alkyl, —C₁₋₆ alkoxy, —C₁₋₆ haloalkyl, and —C₁₋₆ haloalkoxy. In some embodiments, R² is selected from the group consisting of: —CN, —F, —C₁, —Br, —CH₃, —CH₂CH₃, —CF₃, —CF₂CH₃, —OCF₃, —OCHF₂, and -cyclopropyl.

In some embodiments, R³ is hydrogen. In other embodiments, R³ is halo.

In some embodiments, R⁴ is hydrogen. In other embodiments, R⁴ is halo.

In some embodiments, R⁵ is hydrogen. In other embodiments, R⁵ is halo.

In some embodiments, R⁶ is selected from the group consisting of:

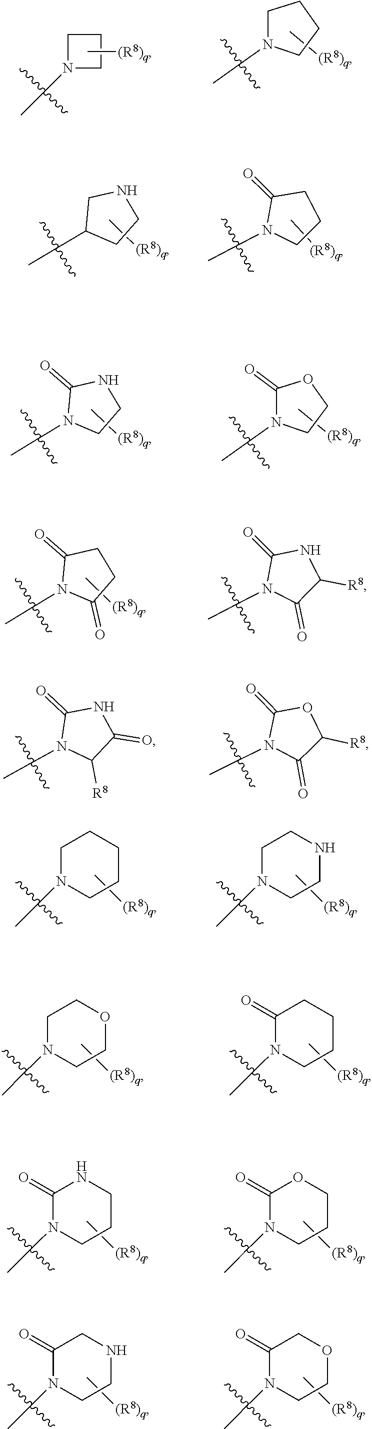

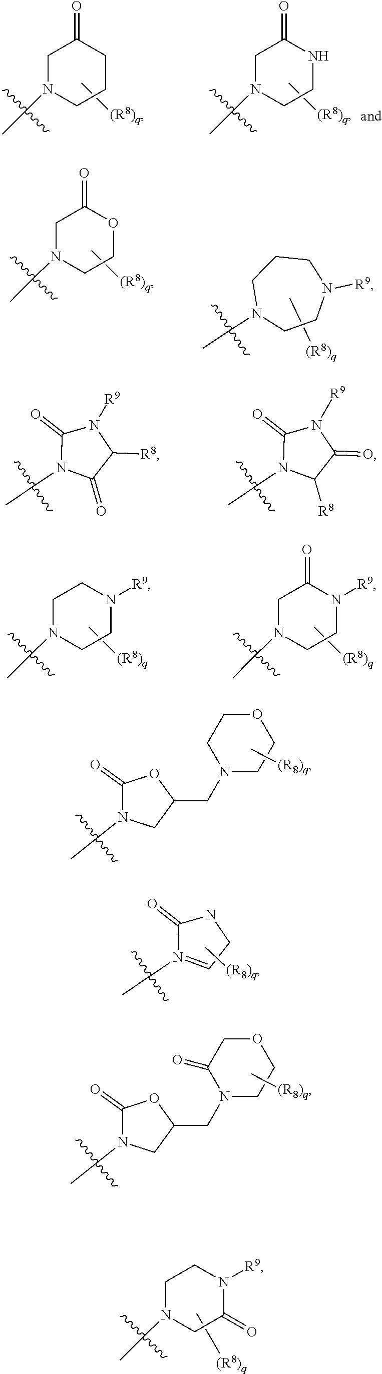

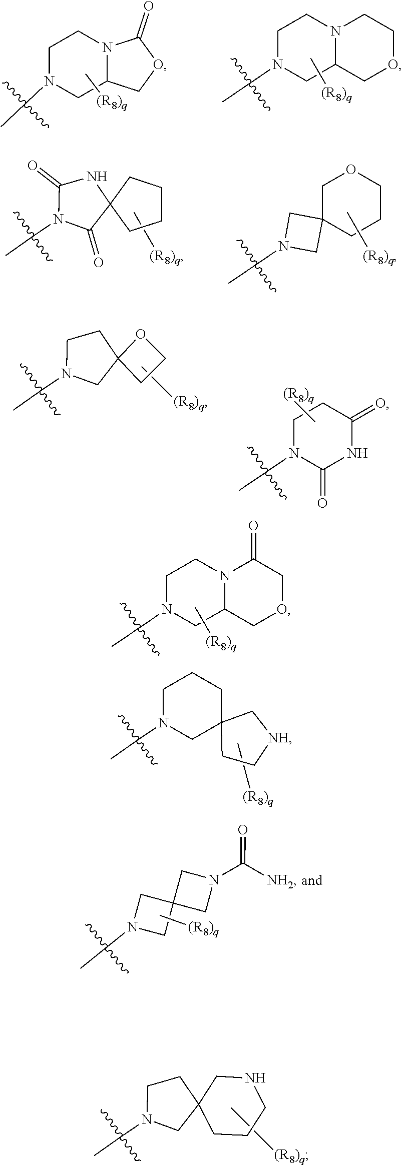

wherein q is an integer value selected from 0, 1, and 2;
wherein each R$ is independently fluorine, phenyl, pyridine, carboxyl, —C(═O)—(C₁-C₆ alkyl), (C₁₋₆ alkyl)-phenyl, —(C₁₋₆ alkyl)-phenyl-O—(C₁-C₆ alkyl), —CF₃, —C₁₋₆ alkyl, —(C₁₋₆ alkyl)-CN, —C₁₋₆ alkenyl, C₃₋₈ cycloalkyl, —(C₁₋₆ alkyl)═OH, —(C₁₋₆ alkyl)-NH₂, —S(═O)₂NH₂, —S(═O)₂—(C₁₋₆ alkyl), —(C₁₋₆ alkyl)-N(CH₃)₂, —(C₁-C₆ alkyl)═O-alkyl, —(C₁-C₆ alkyl)═O—(C₁-C₆ alkyl)═OH, —(C₁₋₆ alkyl)-NH₂, C₁₋₆ alkyl-O-alkenyl, —C(═O) NH₂, —C(═O) OH, —C(═O) OCH₃, —C(═O) NH(C; . . . 6 alkyl), C(═O)N(C₁₋₆ alkyl)₂, —C(═O)—O—(C₁₋₆ alkyl), —(C₁₋₆ alkyl)-NC(═O)—H, —OH, —(C₁₋₆ alkyl)-NC(═O)—CF₃, or —(C₁₋₆ alkyl)-NC(═O)(C₁₋₆ alkyl); and wherein R⁹ is hydrogen, —C₁₋₆ alkyl, —C₃₋₈ cycloalkyl, —(C₁₋₆ alkyl)═OH, —(C₁₋₆ alkyl)-NH₂, or —(C₁₋₆ alkyl)-NC(═O)(C₁₋₆ alkyl).

In some embodiments, R⁶ is selected from the group consisting of:

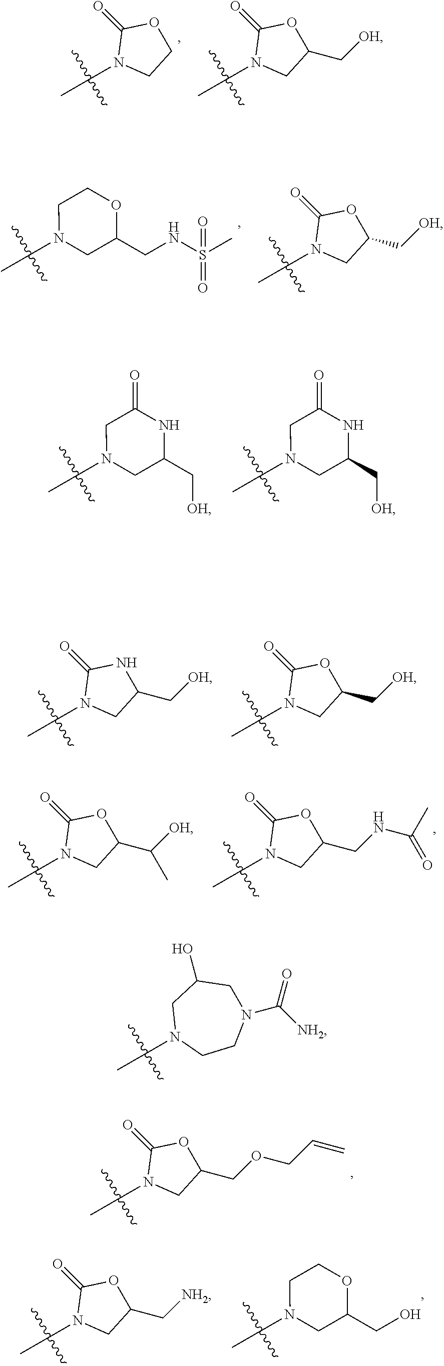

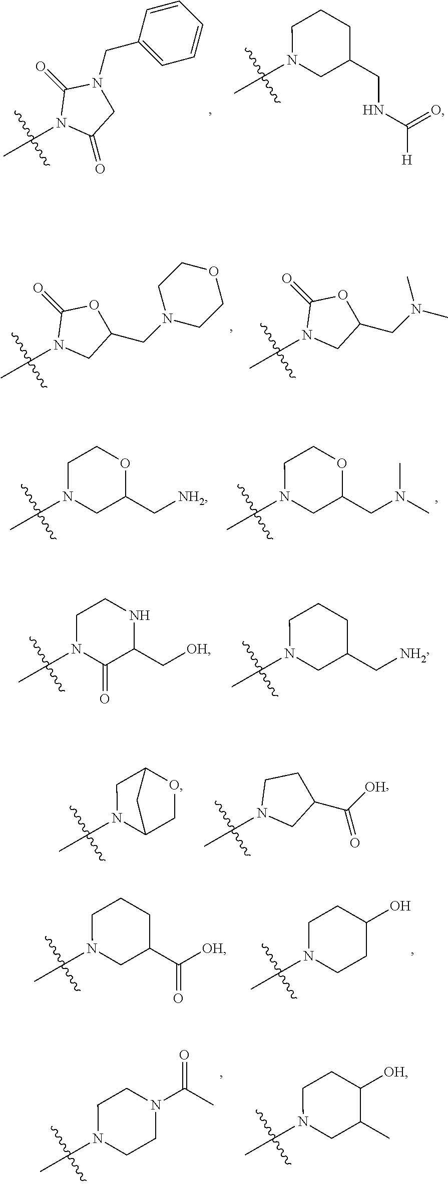

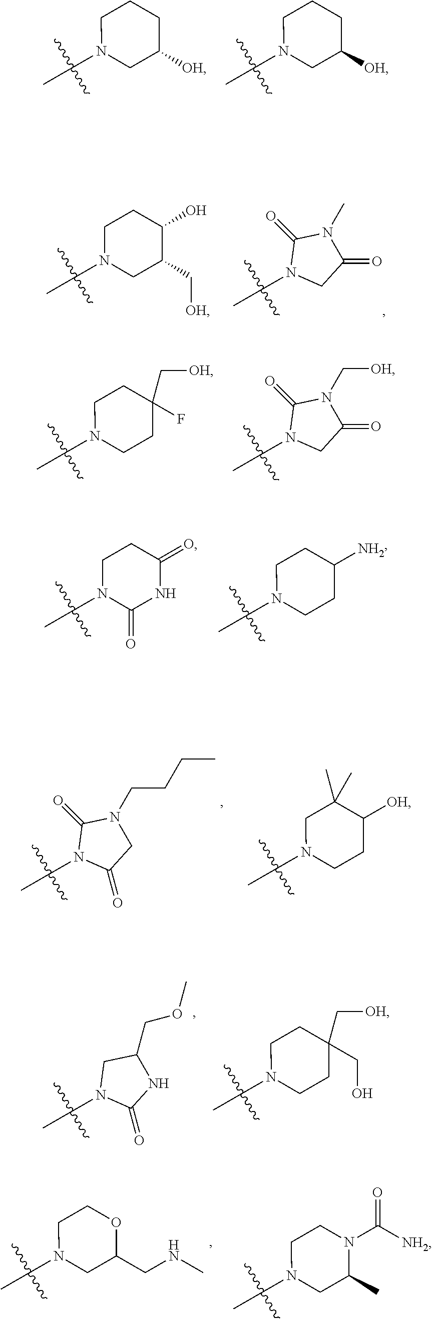

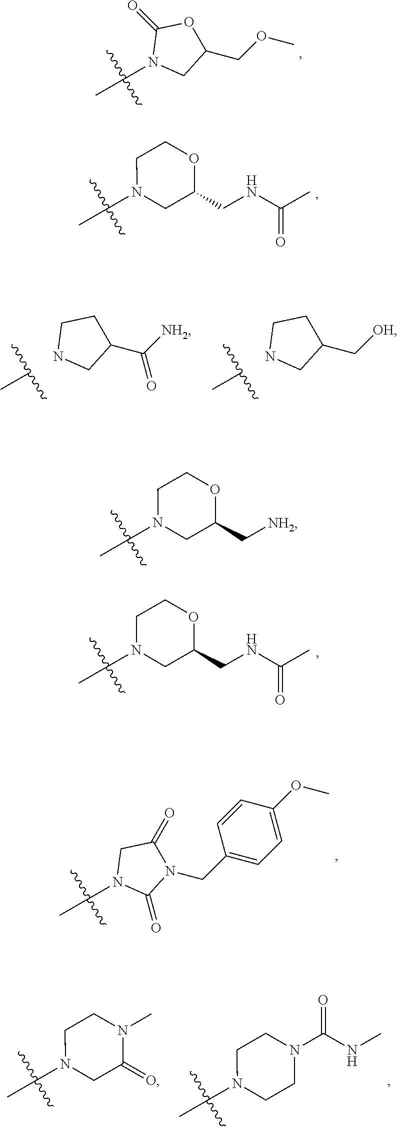

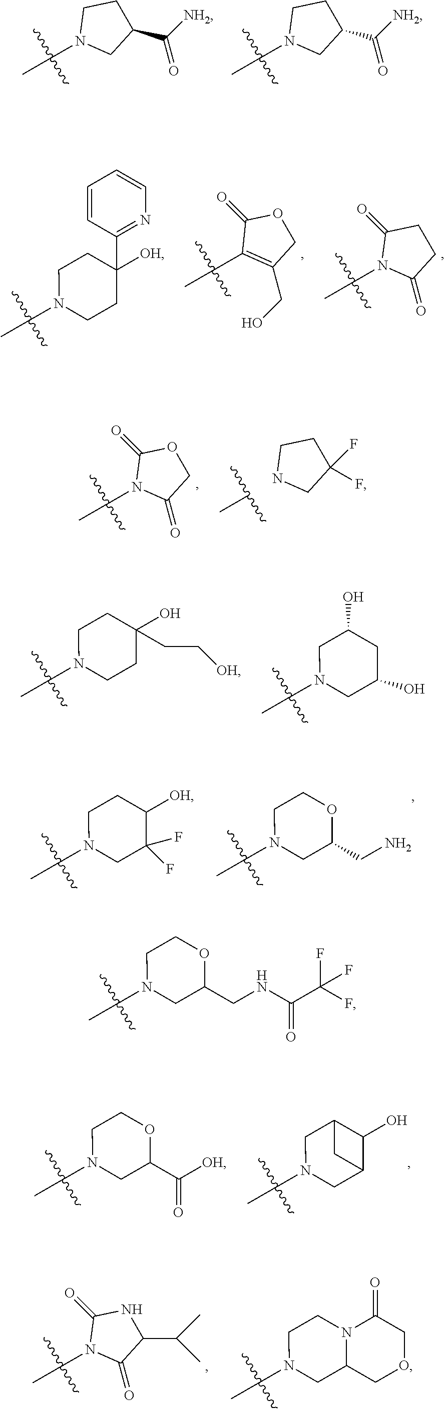

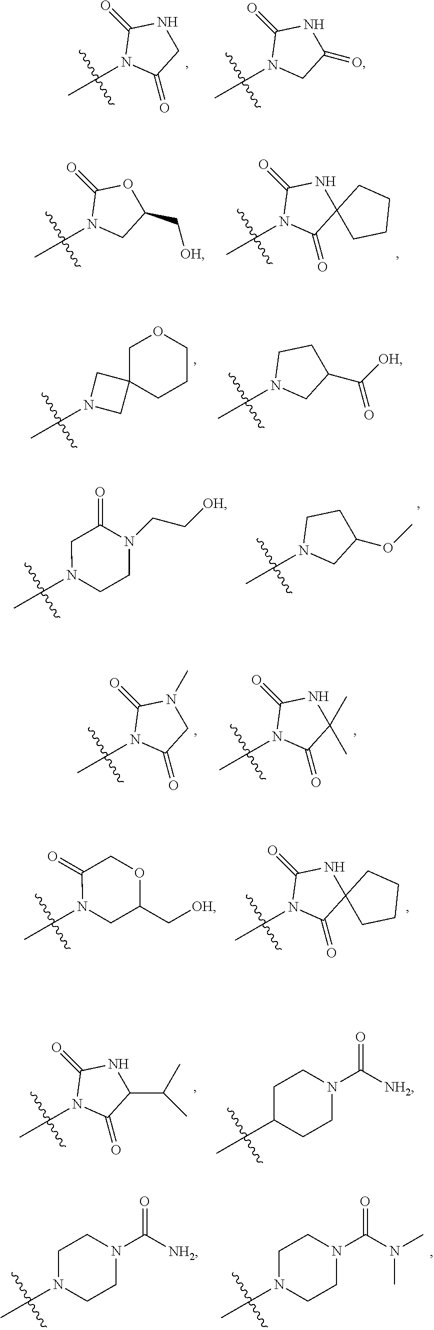

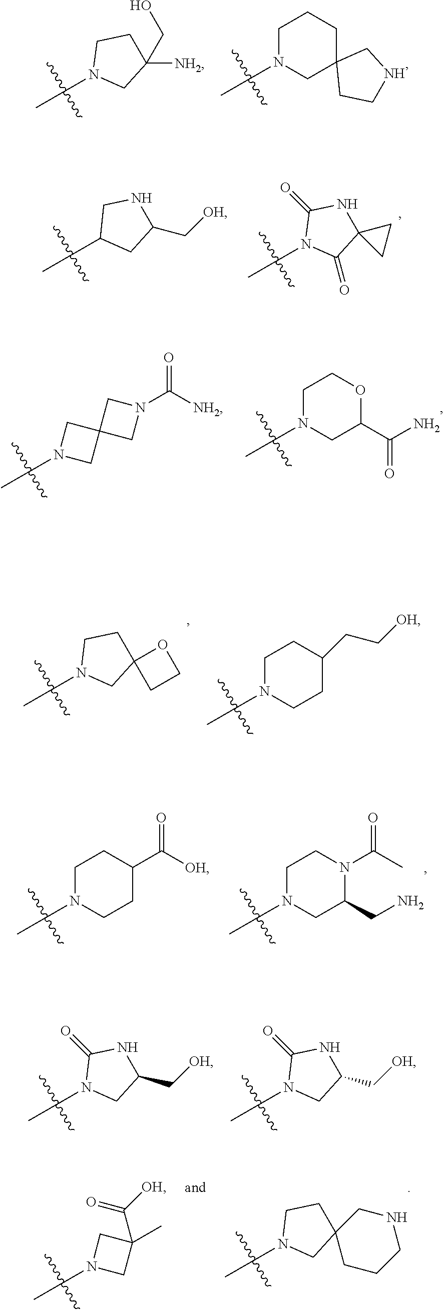

In some embodiments, R⁶ is selected from the group consisting of:

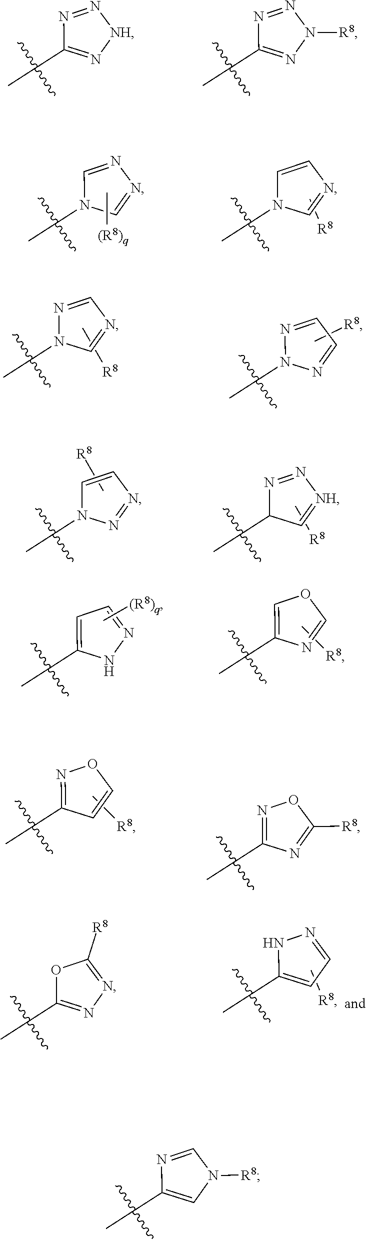

wherein q is an integer value selected from 0, 1, and 2;
wherein each R³ is independently fluorine, phenyl, pyridine, carboxyl, —C(═O)—(C₁-C₆ alkyl), —(C₁₋₆ alkyl)-phenyl, —(C₁₋₆ alkyl)-phenyl-O—(C₁-C₆ alkyl), —CF₃, —C₁₋₆ alkyl, —(C₁₋₆ alkyl)-CN, —C₁₋₆ alkenyl, —C₃₋₈ cycloalkyl, —(C₁₋₆ alkyl)═OH, —(C₁₋₆ alkyl)-NH₂, —S(═O)₂NH₂, —S(═O)₂—(C₁₋₆ alkyl), —(C₁₋₆ alkyl)-N(CH₃)₂, —(C₁-C₆ alkyl)═O-alkyl, —(C₁-C₆ alkyl)═O—(C₁-C₆ alkyl)═OH, —(C₁₋₆ alkyl)-NH₂, C₁₋₆ alkyl-O-alkenyl, —C(═O) NH₂, —C(═O)OH, —C(═O) OCH₃, —C(═O)NH(C₁₋₆ alkyl), C(═O)N(C₁₋₆ alkyl)₂, —C(═O)—O—(C₁₋₆ alkyl), —(C₁₋₆ alkyl)-NC(═O)—H, —OH, —(C₁₋₆ alkyl)-NC(═O)—CF₃, or —(C₁₋₆ alkyl)-NC(═O) (C₁₋₆ alkyl); and
wherein R⁹ is hydrogen, —C₁₋₆ alkyl, —C₃₋₈ cycloalkyl, —(C₁₋₆ alkyl)═OH, —(C₁₋₆ alkyl)-NH₂, or —(C₁₋₆ alkyl)-NC(═O) (C₁₋₆ alkyl).

In some embodiments, R⁶ is selected from the group consisting of:

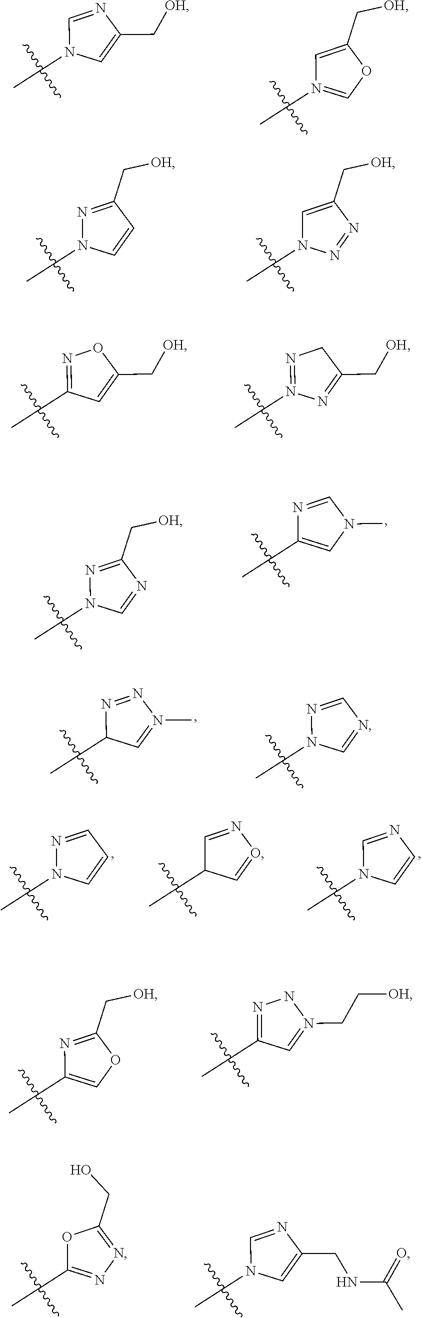

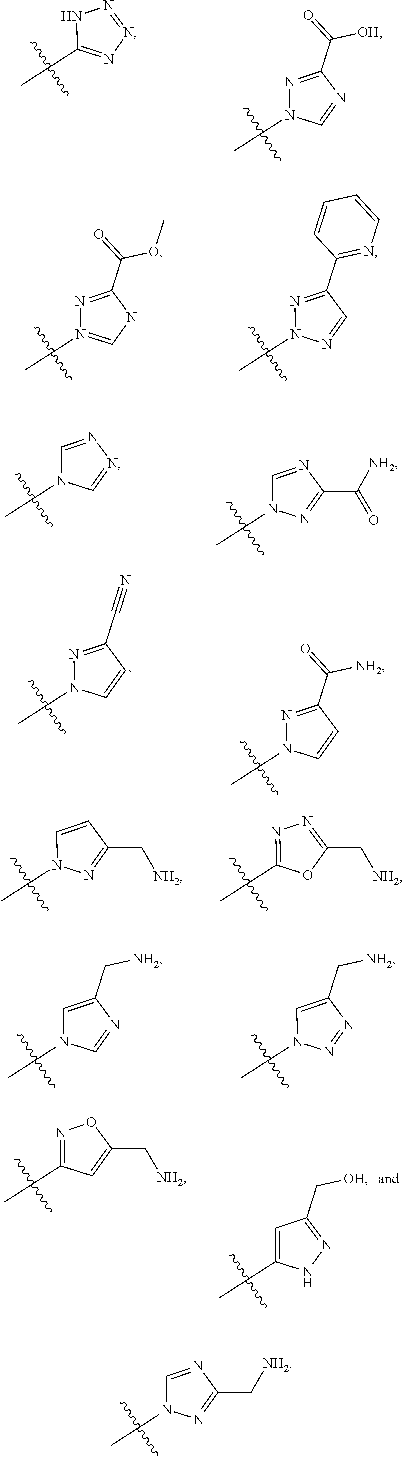

In some embodiments, R⁶ may include a group that is isotopically enriched at one or more atoms, including at the R⁸ group. In some embodiments, one or more hydrogren atoms found in the R⁶ group may be enriched with deuterium.

In some embodiments, the compound is a readthrough modulator disclosed herein, or a pharmaceutically acceptable salt thereof.

In a further embodiment, provided herein is a pharmaceutical composition comprising a therapeutically effective amount of one or more readthrough modulator compounds disclosed herein. In some embodiments, the pharmaceutical composition further comprises an aminoglycoside. In some embodiments, the aminoglycoside is selected from the group consisting of kanamycin A, amikacin, tobramycin, dibekacin, gentamicin, geneticin, sisomicin, netilmicin, neomycin B, neomycin C, paromomycin, streptomycin, plazomicin, tobramycin,

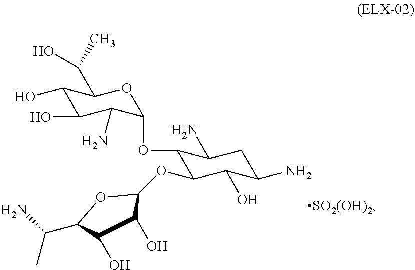

and pharmaceutically acceptable salts thereof. In some specific embodiments, the aminoglycoside is selected from the group consisting of geneticin, ELX-02, and paromomycin. In some embodiments, the pharmaceutical composition of the present disclosure may further comprise a compound or compounds that reduce intracellular levels of elongation release factor eRF3 (GSPT1) or inhibits eRF3 biological function (heretofor termed eRF3 modulator). In some embodiments, the reduction in eRF3 levels can by achieved by compounds that promote eRF3 degradation. Agents that can promote degradation of eRF3 include some cereblon E3 ligase modulating agents, including but not limited to CC-90009.

In a further embodiment, provided herein is a method of enhancing readthrough of genes containing premature termination codons, the method comprising administering to a subject in need thereof: (i) a readthrough modulator; and (ii) an aminoglycoside. In some embodiments, the readthrough modulator is a readthrough modulator disclosed herein. In some embodiments, aminioglycoside is selected from the group consisting of kanamycin A, amikacin, tobramycin, dibekacin, gentamicin, geneticin, sisomicin, netilmicin, neomycin B, neomycin C, paromomycin, streptomycin, plazomicin, tobramycin, ELX-02, and pharmaceutically acceptable salts thereof.

In some embodiments, said compound and aminoglycoside are selected to increase ribosomal readthrough of mRNA transcripts carrying a premature stop codon mutation of the cystic fibrosis CFTR channel. In some embodiments, the mutation is selected from one or more of G542X, R553X, R1162X, and W1282X. In some embodiments, the mutation is R1162X. In other embodiments, the mutation is G542X. In some embodiments, the increase in readthrough is greater than 10 percent as compared to administration of aminoglycoside alone. In other embodiments, the increase in readthrough is greater than 50 percent as compared to administration of aminoglycoside alone. In yet other embodiments, the increase in readthrough is greater than 100 percent as compared to administration of aminoglycoside alone.

In a further embodiment, provided herein is a method of treating cystic fibrosis, the method comprising administering (i) eRF3 modulator, (ii) an aminoglycoside, and (iii) a readthrough modulator to a subject in need thereof. In some embodiments, the readthrough modulator is a readthrough modulator compound disclosed herein. In some embodiments, the aminioglycoside is selected from the group consisting of kanamycin A, amikacin, tobramycin, dibekacin, gentamicin, geneticin, sisomicin, netilmicin, neomycin B, neomycin C, paromomycin, streptomycin, plazomicin, tobramycin, ELX-02, and pharmaceutically acceptable salts thereof. In some specific embodiments, the aminoglycoside is selected from the group consisting of geneticin, ELX-02, and paromomycin. In some embodiments, the eRF3 modulator is a cereblon E3 ligase modulator. In some embodiments the eRF3 modulator is a cereblon E3 disclosed herein.

In some embodiments, the eRF3 modulator, aminoglycoside, and readthrough modulator are selected to increase ribosomal readthrough of mRNA transcripts carrying a premature stop codon mutation of the cystic fibrosis CFTR channel. In some embodiments, the mutation is selected from one or more of G542X, R553X, R1162X, and W1282X. In some embodiments, the mutation is R1162X. In other embodiments, the mutation is G542X. In some embodiments, the increase in readthrough is greater than 10 percent as compared to administration of aminoglycoside alone. In other embodiments, the increase in readthrough is greater than 50 percent as compared to administration of aminoglycoside alone. In yet other embodiments, the increase in readthrough is greater than 100 percent as compared to administration of aminoglycoside alone.

### DETAILED DESCRIPTION

Provided herein are compounds having readthrough modulation activity. In some embodiments, the readthrough modulator compounds provided herein have the structure of Formula (I):

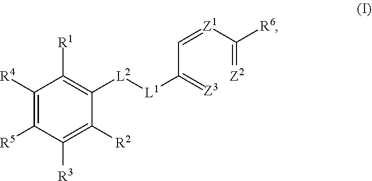

or a pharmaceutically acceptable salt thereof, wherein:
R¹, R², R³, R⁴, and R⁵ are each independently selected from the group consisting of: hydrogen, halo, —CN, —NO₂, —OH, C₁₋₆ alkyl, C₁₋₆ haloalkyl, C₁₋₆ alkoxy, C₁₋₆ haloalkoxy, and C₃₋₈ cycloalkyl;
L¹ is O, NH, or CH₂;
L² is CH₂, CHF, or CF₂;
Z¹ is CH, N, or C—OCH₃;
Z² is CH or N;
Z³ is CH, N, or CF;
R⁶ is optionally substituted heterocyclyl, optionally substituted heteroaryl, or —NR^{7a}R^{7b}; and
R^{7a} and R^{7b} are each independently selected from the group consisting of: hydrogen, optionally substituted C₁₋₆ alkyl, optionally substituted C₃₋₈ cycloalkyl; optionally substituted aryl, optionally substituted heterocyclyl, and optionally substituted heteroaryl.

In some embodiments of Formula (I), when R¹ and R² are both hydrogen and R⁶ is optionally substituted pyrazolyl, then R³ is not halo and R⁴ is not halo.

In some embodiments of Formula (I), L¹ can be O. In other embodiments of Formula (I), L¹ can be N. In yet other embodiments of Formula (I), L¹ can be CH₂. In some embodiments of Formula (I), L² can be CH₂. In some embodiments of Formula (I), L¹ can be O and L² can be CH₂. In other embodiments of Formula (I), L¹ can be O and L² can be CF₂. In yet other embodiments of Formula (I), L¹ can be O and L² can be CHF. In some embodiments of Formula (I), L¹ can be CH₂ and L² can be CH₂.

In some embodiments of Formula (I), Z¹ can be N. In some embodiments of Formula (I), Z¹ can be CH. In some embodiments of Formula (I), Z² can be N. In other embodiments of Formula (I), Z² can be CH. In some embodiments of Formula (I), Z³ can be N. In other embodiments of Formula (I), Z³ can be CH. In yet other embodiments of Formula (I), Z³ can be CF. In some embodiments of Formula (I), Z¹ is N, Z² is CH, and Z³ is CH. In some embodiments of Formula (I), Z¹ is CH, Z² is CH, and Z³ is CH. In other embodiments, of Formula (I), Z¹ is CH, Z² is N, and Z³ is CH. In yet other embodiments, of Formula (I), Z¹ is CH, Z² is CH, and Z³ is N. In some embodiments of Formula (I), Z¹ is CH, Z² is CH, and Z³ is CF. In some embodiments of Formula (I), Z¹ is N, Z² is CH, and Z³ is N. In other embodiments of Formula (I), Z¹ is CH, Z² is N, and Z³ is N.

In some embodiments of Formula (I), when L¹ is O and L² is CH₂, Z¹ can be N. In some embodiments of Formula (I), when L¹ is O and L² is CH₂, Z¹ can be CH. In some embodiments of Formula (I), when L¹ is O and L² is CH₂, Z² can be N. In other embodiments of Formula (I), when L¹ is O and L² is CH₂, Z² can be CH. In some embodiments of Formula (I), when L¹ is O and L² is CH₂, Z³ can be N. In other embodiments of Formula (I), when L¹ is O and L² is CH₂, Z³ can be CH. In yet other embodiments of Formula (I), when L¹ is O and L² is CH₂, Z³ can be CF. In some embodiments of Formula (I), when L¹ is O and L² is CH₂, Z¹ is N, Z² is CH, and Z³ is CH. In some embodiments of Formula (I), when L¹ is O and L² is CH₂, Z¹ is CH, Z² is CH, and Z³ is CH. In other embodiments, of Formula (I), when L¹ is O and L² is CH₂, Z¹ is CH, Z² is N, and Z³ is CH. In yet other embodiments, of Formula (I), when L¹ is O and L² is CH₂, Z¹ is CH, Z² is CH, and Z³ is N. In some embodiments of Formula (I), when L¹ is O and L² is CH₂, Z¹ is CH, Z² is CH, and Z³ is CF. In some embodiments of Formula (I), when L¹ is O and L² is CH₂, Z¹ is N, Z² is CH, and Z³ is N. In some embodiments of Formula (I), when L¹ is O and L² is CH₂, Z¹ is CH, Z² is N, and Z³ is N.

In some embodiments of Formula (I), when L¹ is O and L² is CF₂, Z¹ can be N. In some embodiments of Formula (I), when L¹ is O and L² is CF₂, Z¹ can be CH. In some embodiments of Formula (I), when L¹ is O and L² is CF₂, Z² can be N. In other embodiments of Formula (I), when L¹ is O and L² is CF₂, Z² can be CH. In some embodiments of Formula (I), when L¹ is O and L² is CF₂, Z³ can be N. In other embodiments of Formula (I), when L¹ is O and L² is CF₂, 73 can be CH. In yet other embodiments of Formula (I), when L¹ is O and L² is CF₂, Z³ can be CF. In some embodiments of Formula (I), when L¹ is O and L² is CF₂, Z¹ is N, Z² is CH, and Z³ is CH. In some embodiments of Formula (I), when L¹ is O and L² is CF₂, Z¹ is CH, Z² is CH, and Z³ is CH. In other embodiments, of Formula (I), when L¹ is O and L² is CF₂, Z¹ is CH, Z² is N, and Z³ is CH. In yet other embodiments, of Formula (I), when L¹ is O and L² is CF₂, Z¹ is CH, Z² is CH, and Z³ is N. In some embodiments of Formula (I), when L¹ is O and L² is CF₂, Z¹ is CH, Z² is CH, and Z³ is CF.

In some embodiments of Formula (I), R¹ may be halo, —CN, —C₁₋₆ alkyl, —C₁₋₆ alkoxy, —C₁₋₆ haloalkyl, —C₁₋₆ haloalkoxy, or -cyclopropyl. In some embodiments, R¹ may be halo. In some specific embodiments, R¹ may be —F, —C₁, —Br, or —I. In other embodiments, R¹ may be —C₁₋₆ alkyl. In some specific embodiments, R¹ may be —CH₃ or —CH₂CH₃. In other embodiments, R¹ may be —C₁₋₆ haloalkyl. In some specific embodiments, R¹ may be —CF₃ or —CF₂CH₃. In some embodiments, R¹ may be —C₁₋₆ alkoxy. In some specific embodiments, R¹ may be —OCH₃ or —OCH₂CH₃. In other embodiments, R¹ may be —C₁₋₆ haloalkoxy. In some specific embodiments, R¹ may be —OCF₃ or —OCHF₂.

In some embodiments of Formula (I), R² may be halo, —CN, —C₁₋₆ alkyl, —C₁₋₆ alkoxy, —C₁₋₆ haloalkyl, —C₁₋₆ haloalkoxy, or -cyclopropyl. In some embodiments, R² may be halo. In some specific embodiments, R² may be —F, —C₁, —Br, or —I. In other embodiments, R² may be —C₁₋₆ alkyl. In some specific embodiments, R¹ may be —CH₃ or —CH₂CH₃. In other embodiments, R² may be —C₁₋₆ haloalkyl. In some specific embodiments, R² may be —CF₃ or —CF₂CH₃. In some embodiments, R² may be —C₁₋₆ alkoxy. In some specific embodiments, R² may be —OCH₃ or —OCH₂CH₃. In other embodiments, R² may be —C₁₋₆ haloalkoxy. In some specific embodiments, R² may be —OCF₃ or —OCHF₂.

In some embodiments of Formula (I), R³ may be hydrogen. In some embodiments of Formula (I), R³ may be halo. In some embodiments, R³ may be —F, —C₁, or Br.

In some embodiments of Formula (I), R⁴ may be hydrogen. In some embodiments of Formula (I), R⁴ may be halo. In some embodiments, R⁴ may be —F, —C₁, or Br.

In some embodiments of Formula (I), R⁵ may be hydrogen. In some embodiments of Formula (I), R⁵ may be halo. In some embodiments, R⁵ may be —F, —C₁, or Br.

In some embodiments of Formula (I), R¹ may be halo and R² may be —C₁₋₆ alkyl. In some embodiments of Formula (I), R¹ may be -halo and R² may be —C₁₋₆ alkoxy. In some embodiments of Formula (I), R¹ may be -halo and R² may be —C₁₋₆ haloalkoxy. In some embodiments of Formula (I), R¹ may be —C₁₋₆ alkyl and R² may be —C₁₋₆ haloalkoxy. In some embodiments of Formula (I), R¹ may be —C₁₋₆ alkyl and R² may be —C₁₋₆ haloalkyl. In some embodiments of Formula (I), R¹ may be -halo and R² may be —C₁₋₆ haloalkyl. In some embodiments of Formula (I), R¹ may be -cyclopropyl and R² may be -halo. In some embodiments of Formula (I), R¹ may be —CN and R² may be -halo. In some embodiments of Formula (I), R¹ may be —C₁₋₆ alkyl and R² may be —C₁₋₆ alkyl.

In some embodiments of Formula (I), R¹ may be —C₁ and R² may be —C₁. In some embodiments of Formula (I), R¹ may be —CF₃ and R² may be —CH₃. In some embodiments of Formula (I), R¹ may be —CF₃ and R² may be —CH₂CH₃. In some embodiments of Formula (I), R¹ may be —C₁ and R² may be —CF₃. In some embodiments of Formula (I), R¹ may be —F and R² may be —CF₃. In some embodiments of Formula (I), R¹ may be —Br and R² may be —Br. In some embodiments of Formula (I), R¹ may be —F and R² may be —F. In some embodiments of Formula (I), R¹ may be —CH₃ and R² may be —CH₃. In some embodiments of Formula (I), R¹ may be —CN and R² may be —C₁. In some embodiments of Formula (I), R¹ may be —OCH₃ and R² may be —C₁. In some embodiments of Formula (I), R¹ may be —OCH₃ and R² may be —Br. In some embodiments of Formula (I), R¹ may be —OCHF₂ and R² may be —C₁. In some embodiments of Formula (I), R¹ may be —CF₃ and R² may be —OCF₃. In some embodiments of Formula (I), R¹ may be —CH₃ and R² may be —OCHF₂.

In some embodiments of Formula (I), R⁶ may be selected from

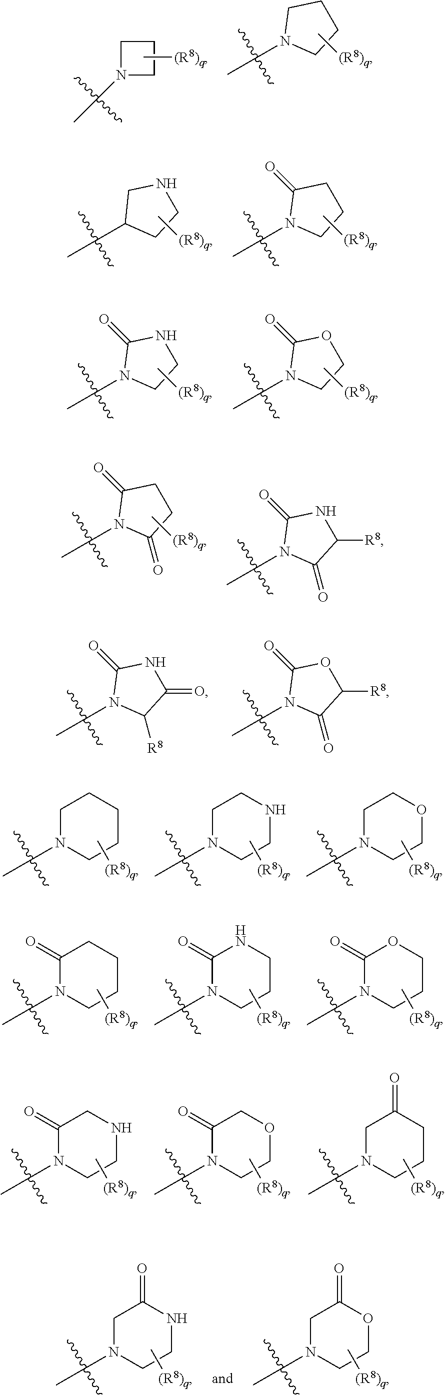

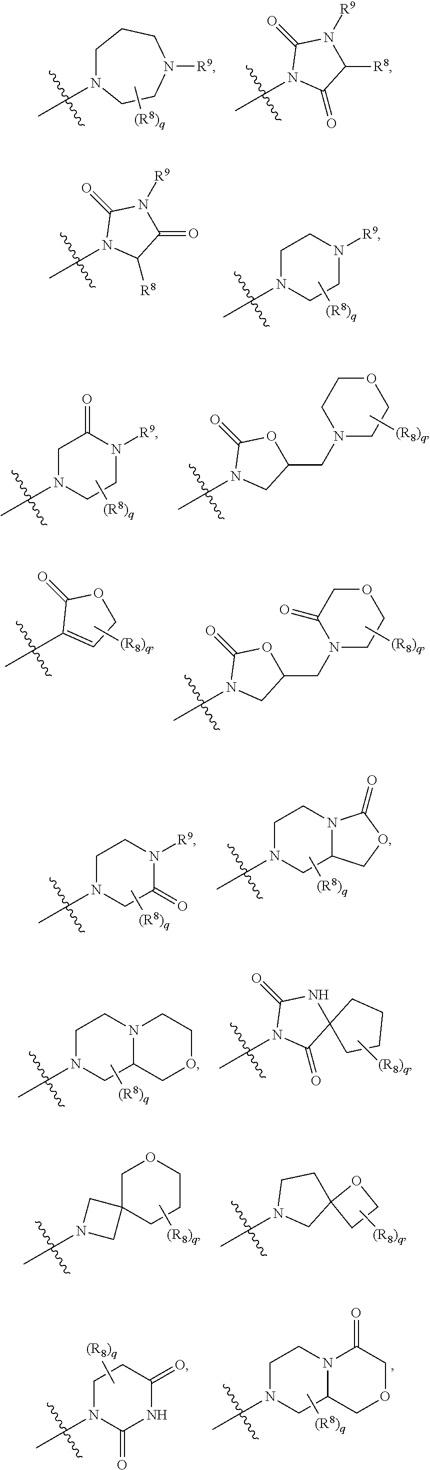

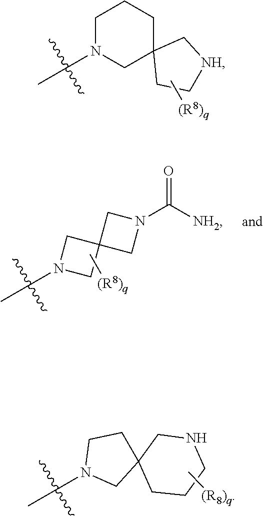

In other embodiments, R⁶ may be selected from

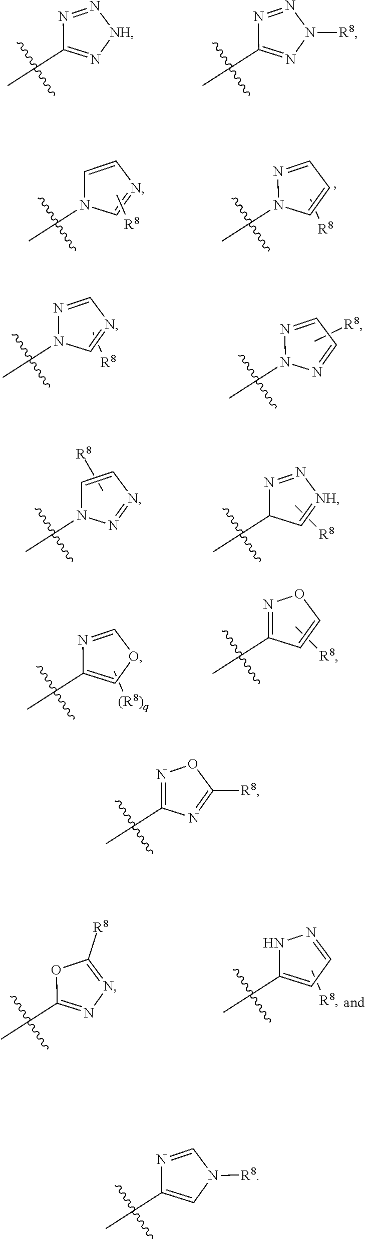

In some embodiments of Formula (I), each R⁸ may be independently fluorine, phenyl, carboxyl, —C(═O)—(C₁-C₆ alkyl), —(C₁₋₆ alkyl)-phenyl, —(C₁₋₆ alkyl)-phenyl-O—(C₁-C₆ alkyl), —CF₃, —C₁₋₆ alkyl, —C₁₋₆ alkenyl, —C₃₋₈ cycloalkyl, —(C₁₋₆ alkyl)═OH, —(C₁₋₆ alkyl)-NH₂, —S(═O)₂NH₂, —S(═O)₂—(C₁₋₆ alkyl), —(C₁₋₆ alkyl)-N(CH₃)₂, —(C₁-C₆ alkyl)═O-alkyl, —(C₁-C₆ alkyl)═O—(C₁₋₆ alkyl)═OH, —(C₁₋₆ alkyl)-NH₂, C₁₋₆ alkyl-O-alkenyl, —C(═O)NH₂, —OH, —C(═O)NH(C₁₋₆ alkyl), C(═O)N(C₁₋₆ alkyl)₂, —C(═O)—O—(C₁₋₆ alkyl), —(C₁₋₆ alkyl)-NC(═O)—H, —(C₁₋₆ alkyl)-NC(═O)—CF₃, or —(C₁₋₆ alkyl)-NC(═O) (C₁₋₆ alkyl). In some specific embodiments, each R⁸ may be —CH₂OH. In other specific embodiments, each R⁸ may be —C(═O) NH₂. In some embodiments, one or more atoms of each R⁸ may be isotopically enriched.

In some embodiments of Formula (I), R⁶ may be defined as above and q may be 0. In other embodiments of Formula (I), R⁶ and R⁸ may be defined as above and q may be 1. In some embodiments of Formula (I), R⁶ and R⁸ may be defined as above and q may be 2.

In some embodiments of Formula (1), R⁹ may be hydrogen. In some embodiments of Formula (I), R⁹ may be —C₁₋₆ alkyl. In some embodiments of Formula (I), R⁹ may be —C₃₋₈ cycloalkyl. In some embodiments of Formula (I), R⁹ may be —(C₁₋₆ alkyl)═OH. In some embodiments of Formula (I), R⁹ may be —(C₁₋₆ alkyl)-NH₂. In some embodiments of Formula (D), R⁹ may be —(C₁₋₆ alkyl)-NC(═O) (C₁₋₄ alkyl).

In some embodiments of Formula (I), R⁶ may be

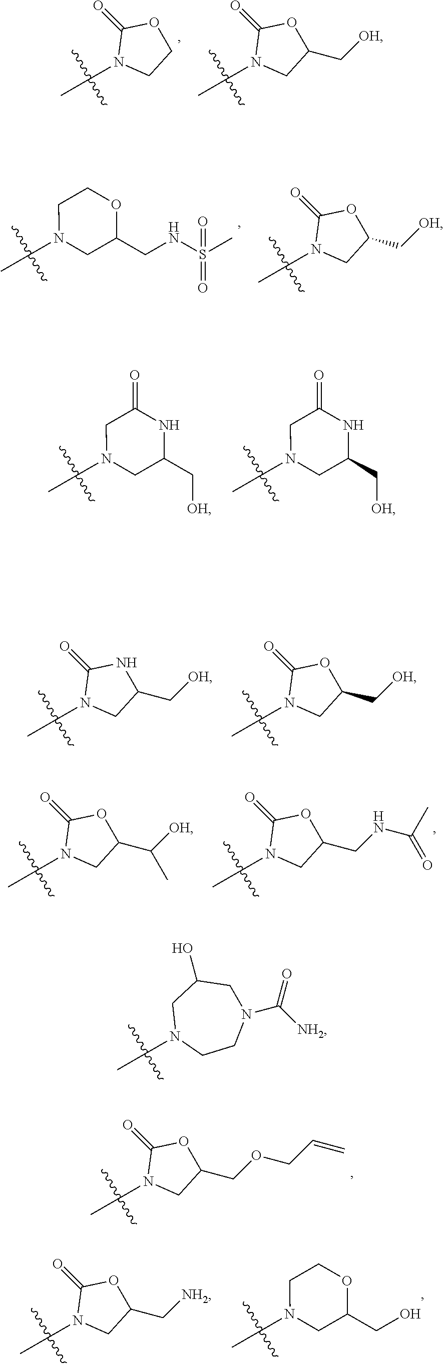

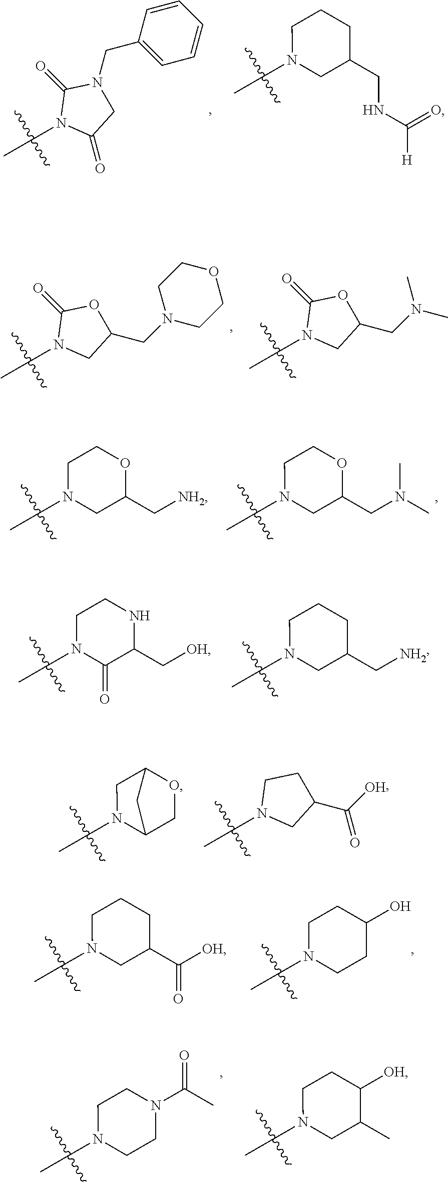

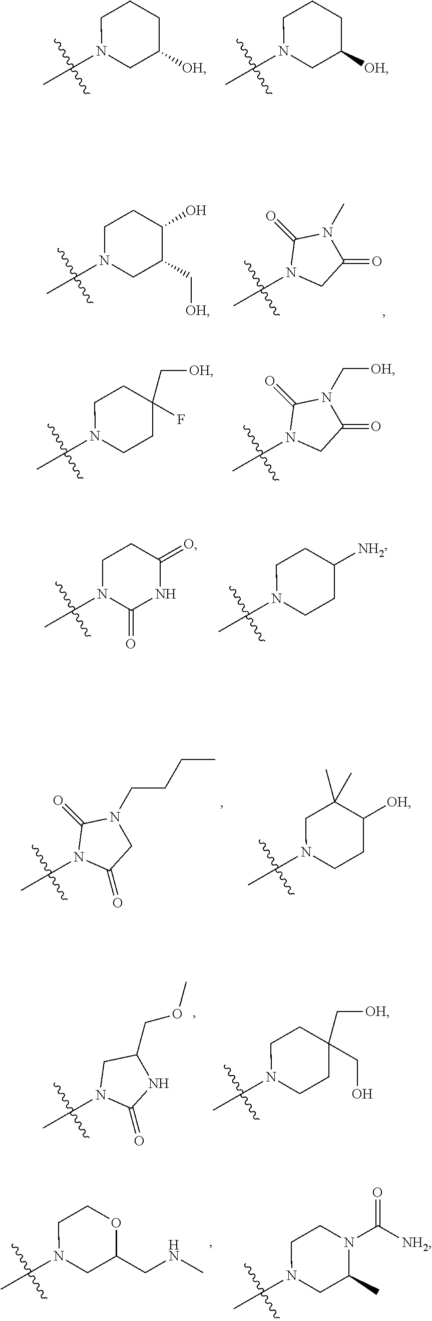

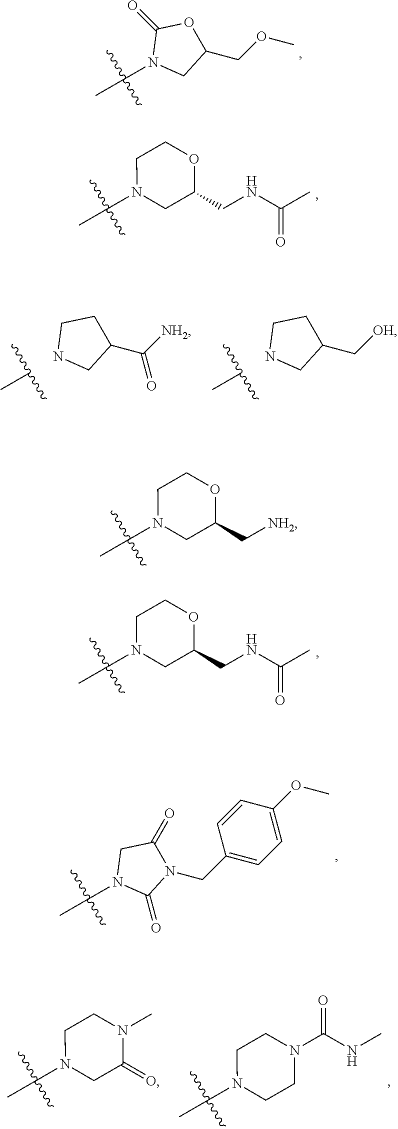

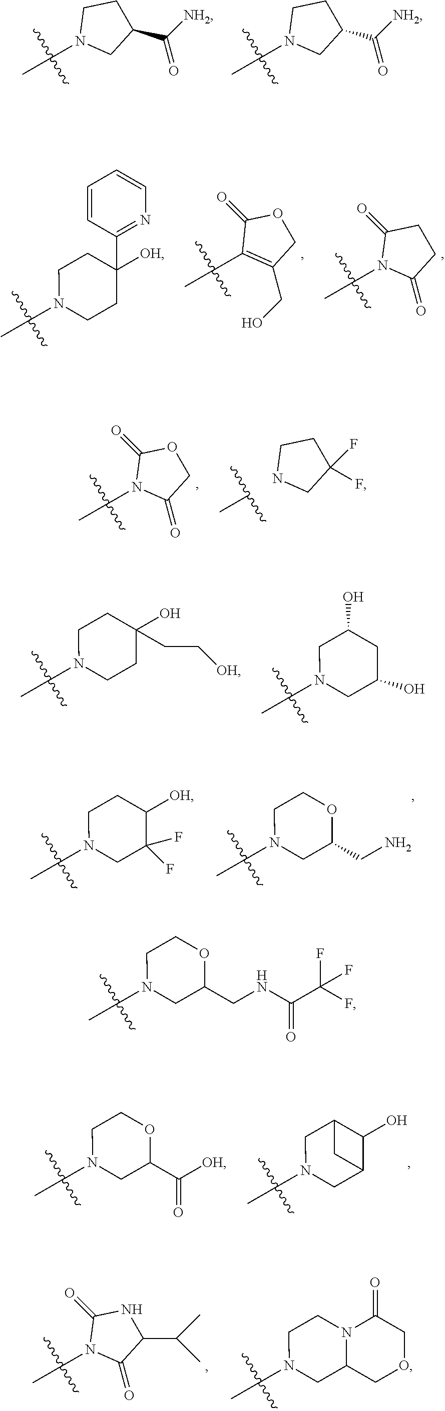

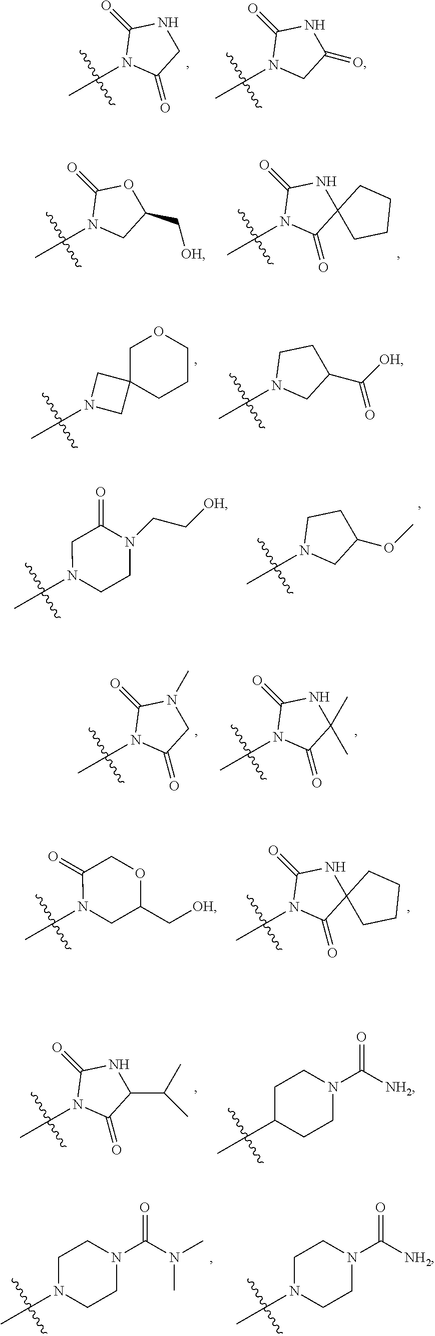

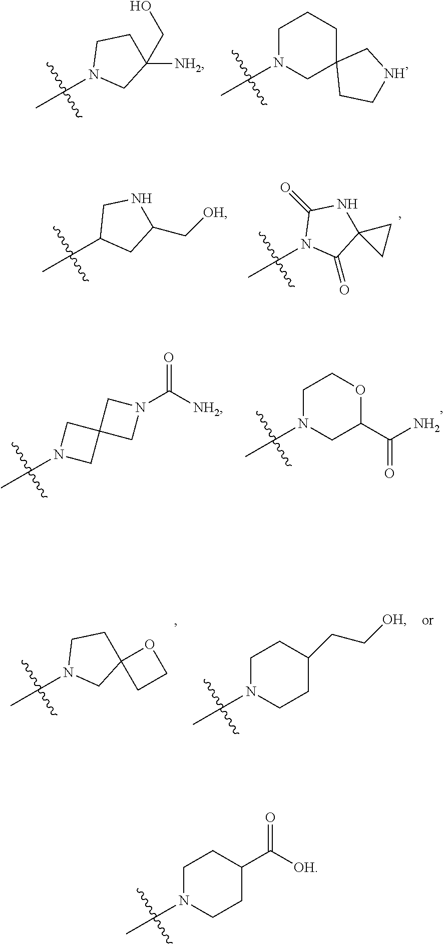

In other embodiments of Formula (I), R⁶ may be selected from the group consisting of:

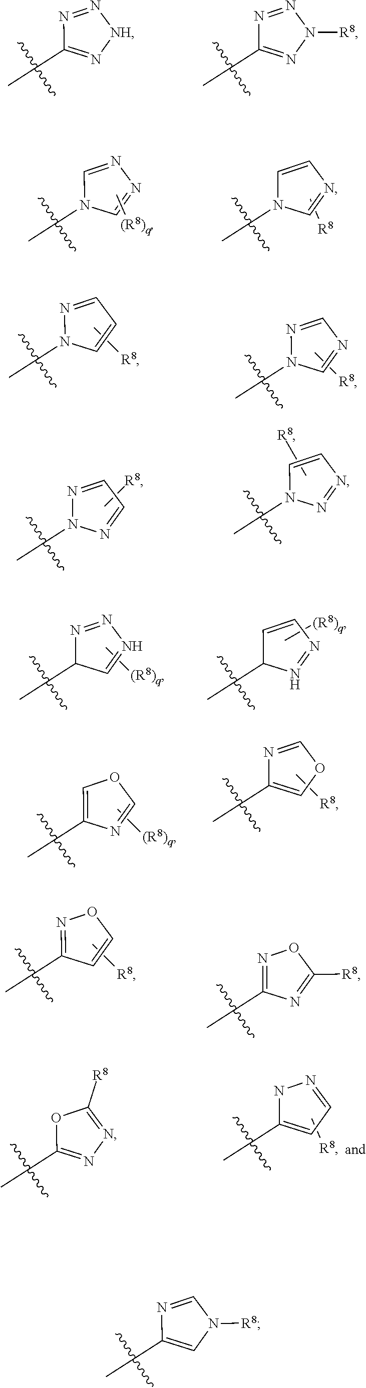

In some embodiments, q is selected from 0, 1, and 2. In some embodiments, each R⁸ is independently fluorine, phenyl, pyridine, carboxyl, —C(═O)—(C₁-C₆ alkyl), —(C₁₋₆ alkyl)-phenyl, —(C₁₋₆ alkyl)-phenyl-O—(C₁-C₆ alkyl), —CF₃, —C₁₋₆ alkyl, —(C₁₋₆ alkyl)-CN, —C₁₋₆ alkenyl, —C₃₋₈ cycloalkyl, —(C₁₋₆ alkyl)═OH, —(C₁₋₆ alkyl)-NH₂, —S(═O)₂NH₂, —S(═O)₂—(C₁₋₆ alkyl), —(C₁₋₆ alkyl)-N(CH₃)₂, —(C₁-C₆ alkyl)═O-alkyl, —(C₁-C₆ alkyl)═O—(C₁-C₆ alkyl)═OH, —(C₁₋₆ alkyl)-NH₂, C₁₋₆ alkyl-O-alkenyl, —C(═O) NH₂, —C(═O)OH, —C(═O) OCH₃, —C(═O)NH(C₁₋₆ alkyl), C(═O)N(C₁₋₆ alkyl)₂, —C(═O)—O—(C₁₋₆ alkyl), —(C₁₋₆ alkyl)-NC(═O)—H, —OH, —(C₁₋₆ alkyl)-NC(═O)—CF₃, or —(C₁₋₆ alkyl)-NC(═O) (C₁₋₆ alkyl). In some embodiments, R⁹ is hydrogen, —C₁₋₆ alkyl, —C₃₋₈ cycloalkyl, —(C₁₋₆ alkyl)═OH, —(C₁₋₆ alkyl)-NH₂, or —(C₁₋₆ alkyl)-NC(═O)(C₁₋₆ alkyl).

In some embodiments, the readthrough modulator compounds describe herein may be selected from the group consisting of:

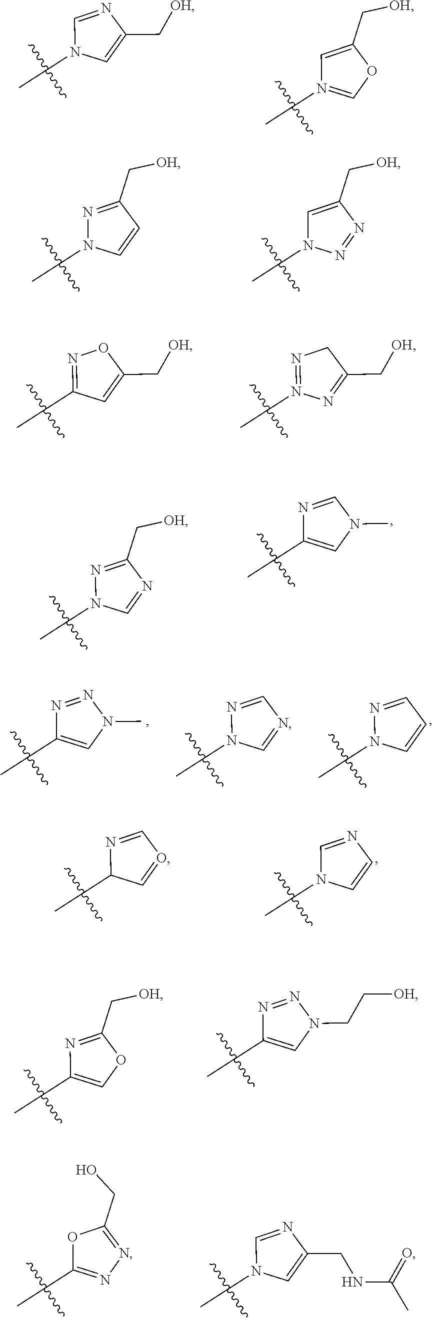

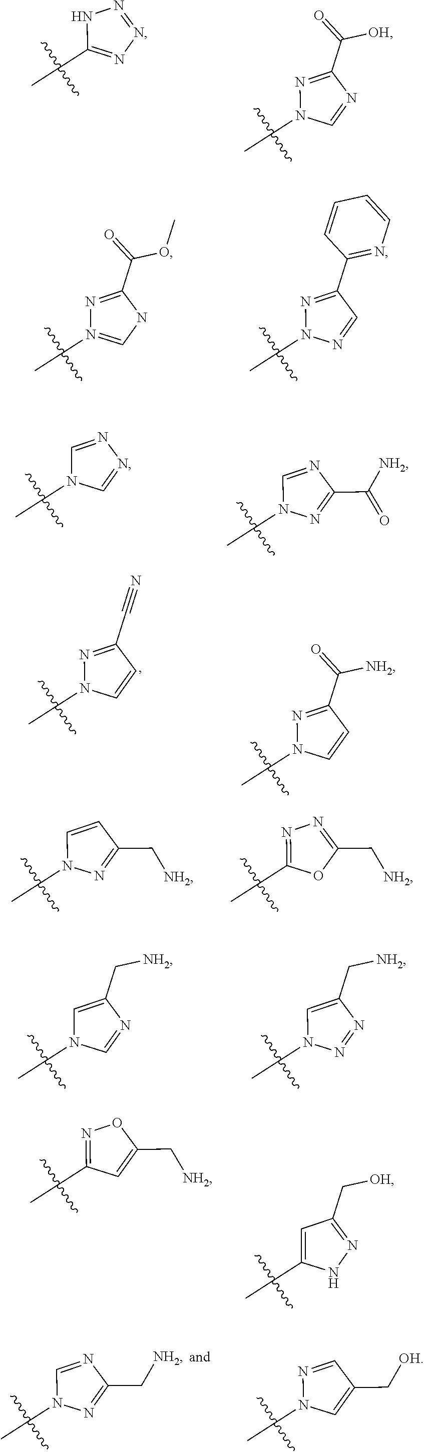

In some embodiments, the compound of Formula (I) includes the proviso that when R¹ and R² are both hydrogen and R⁶ is optionally substituted pyrazolyl, then R³ is not halo and R⁴ is not halo.

In some embodiments, the readthrough modulator compounds describe herein may be selected from the group consisting of:

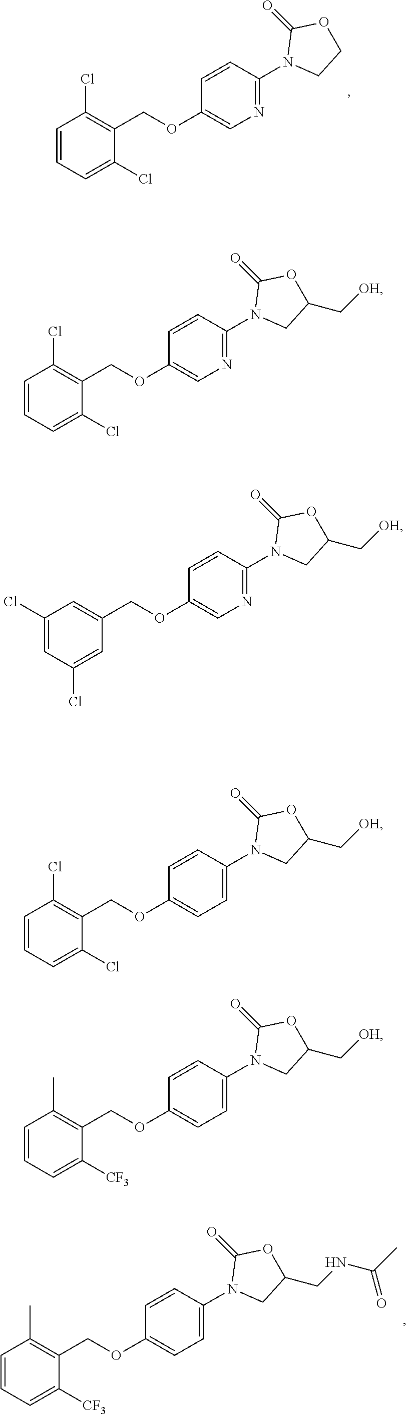 [SMILES: O=C1OCCN1c1ccc(OCc2c(Cl)cccc2Cl)cn1 ; O=C1OC(CO)CN1c1ccc(OCc2c(Cl)cccc2Cl)cn1 ; O=C1OC(CO)CN1c1ccc(OCc2cc(Cl)cc(Cl)c2)cn1 ; O=C1OC(CO)CN1c1ccc(OCc2c(Cl)cccc2Cl)cc1 ; Cc1cccc(C(F)(F)F)c1COc1ccc(N2CC(CO)OC2=O)cc1 ; CC(=O)NCC1CN(c2ccc(OCc3c(C)cccc3C(F)(F)F)cc2)C(=O)O1]

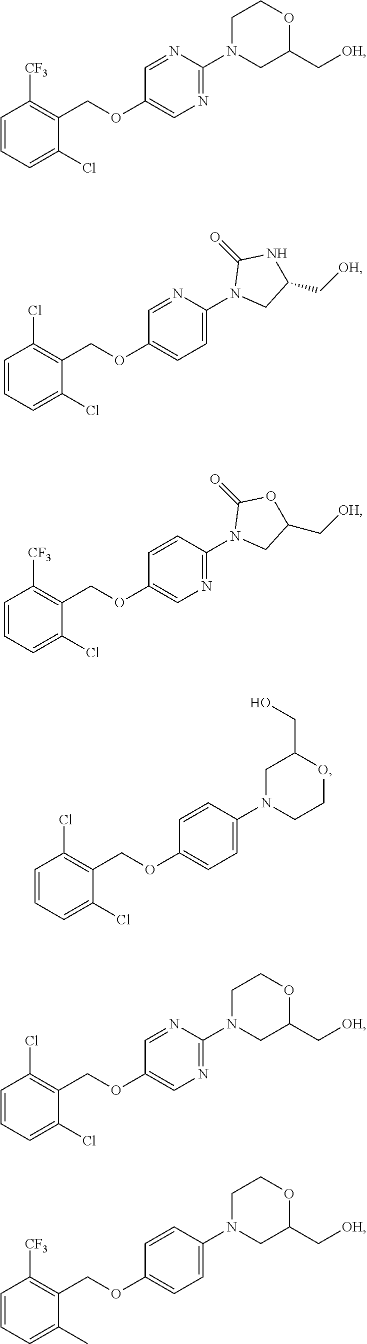 [SMILES: OCC1CN(c2ncc(OCc3c(Cl)cccc3C(F)(F)F)cn2)CCO1 ; O=C1N[C@H](CO)CN1c1ccc(OCc2c(Cl)cccc2Cl)cn1 ; O=C1OC(CO)CN1c1ccc(OCc2c(Cl)cccc2C(F)(F)F)cn1 ; OCC1CN(c2ccc(OCc3c(Cl)cccc3Cl)cc2)CCO1 ; OCC1CN(c2ncc(OCc3c(Cl)cccc3Cl)cn2)CCO1 ; Cc1cccc(C(F)(F)F)c1COc1ccc(N2CCOC(CO)C2)cc1]

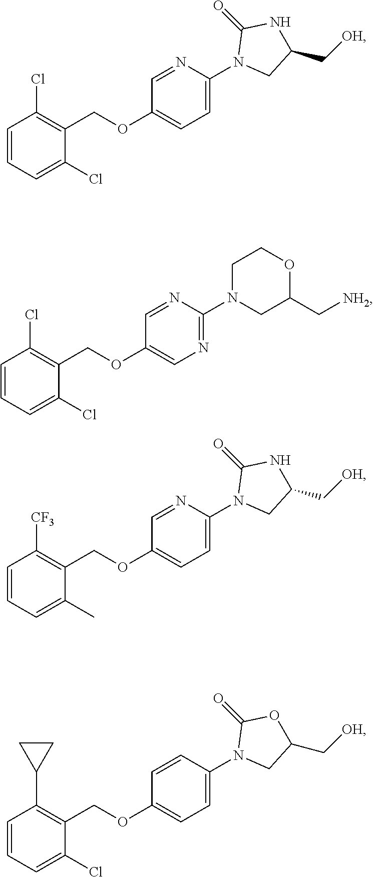 [SMILES: O=C1N[C@@H](CO)CN1c1ccc(OCc2c(Cl)cccc2Cl)cn1 ; NCC1CN(c2ncc(OCc3c(Cl)cccc3Cl)cn2)CCO1 ; Cc1cccc(C(F)(F)F)c1COc1ccc(N2C[C@@H](CO)NC2=O)nc1 ; O=C1OC(CO)CN1c1ccc(OCc2c(Cl)cccc2C2CC2)cc1]

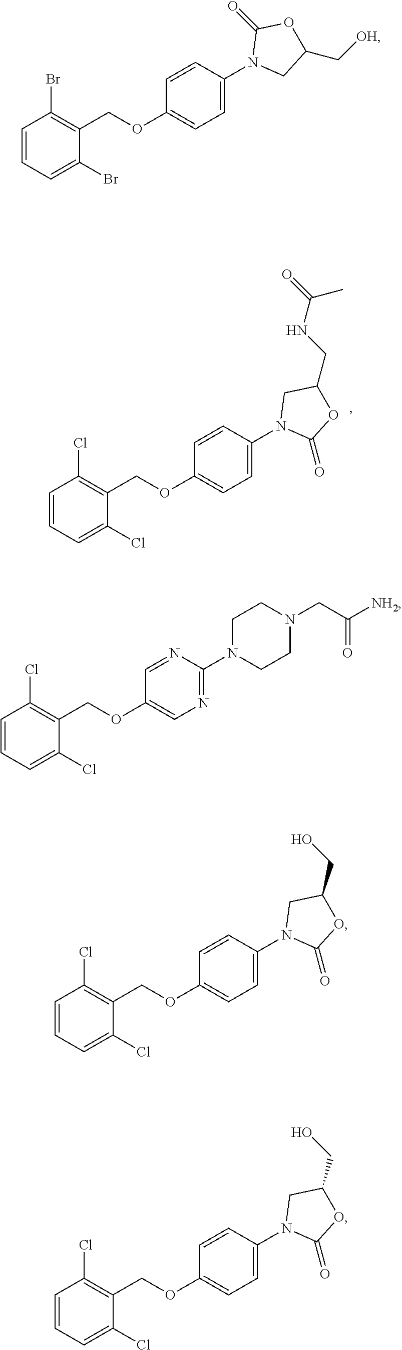 [SMILES: O=C1OC(CO)CN1c1ccc(OCc2c(Br)cccc2Br)cc1 ; CC(=O)NCC1CN(c2ccc(OCc3c(Cl)cccc3Cl)cc2)C(=O)O1 ; NC(=O)CN1CCN(c2ncc(OCc3c(Cl)cccc3Cl)cn2)CC1 ; O=C1O[C@H](CO)CN1c1ccc(OCc2c(Cl)cccc2Cl)cc1 ; O=C1O[C@@H](CO)CN1c1ccc(OCc2c(Cl)cccc2Cl)cc1]

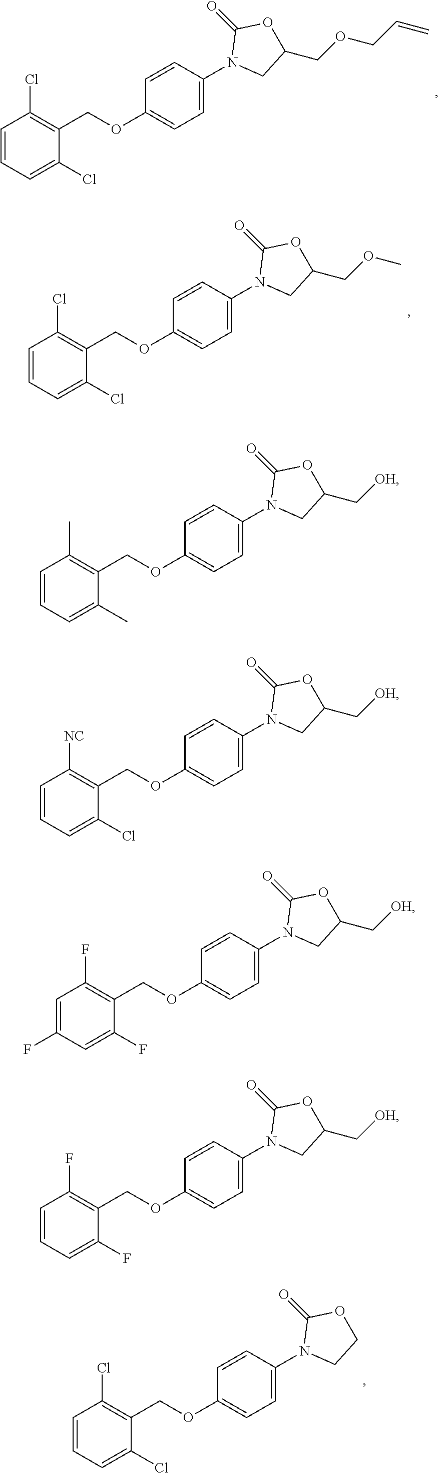

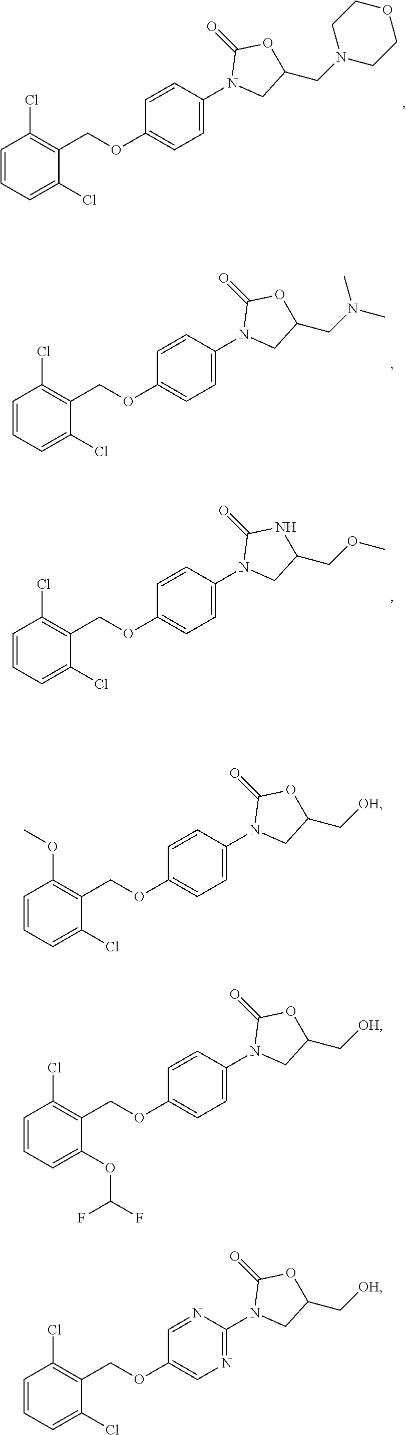 [SMILES: O=C1OC(CN2CCOCC2)CN1c1ccc(OCc2c(Cl)cccc2Cl)cc1 ; CN(C)CC1CN(c2ccc(OCc3c(Cl)cccc3Cl)cc2)C(=O)O1 ; COCC1CN(c2ccc(OCc3c(Cl)cccc3Cl)cc2)C(=O)N1 ; COc1cccc(Cl)c1COc1ccc(N2CC(CO)OC2=O)cc1 ; O=C1OC(CO)CN1c1ccc(OCc2c(Cl)cccc2OC(F)F)cc1 ; O=C1OC(CO)CN1c1ncc(OCc2c(Cl)cccc2Cl)cn1]

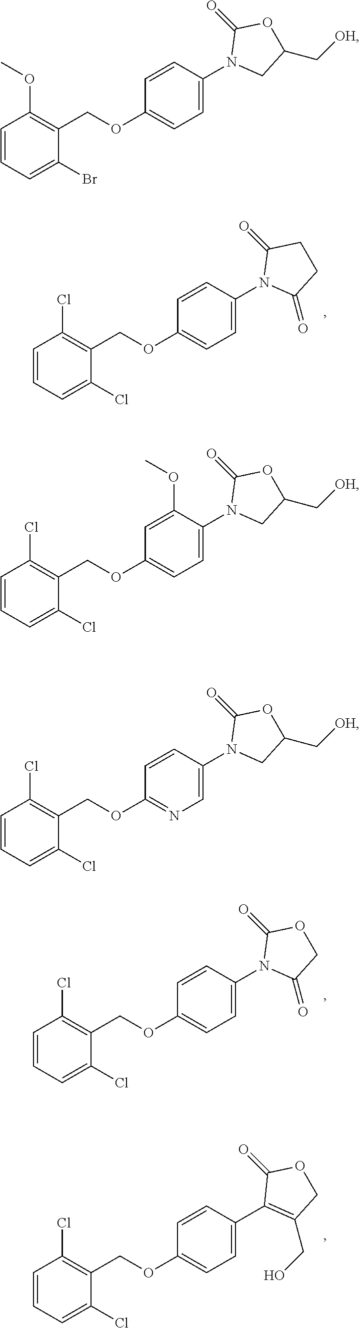 [SMILES: COc1cccc(Br)c1COc1ccc(N2CC(CO)OC2=O)cc1 ; O=C1CCC(=O)N1c1ccc(OCc2c(Cl)cccc2Cl)cc1 ; COc1cc(OCc2c(Cl)cccc2Cl)ccc1N1CC(CO)OC1=O ; O=C1OC(CO)CN1c1ccc(OCc2c(Cl)cccc2Cl)nc1 ; O=C1COC(=O)N1c1ccc(OCc2c(Cl)cccc2Cl)cc1 ; O=C1OCC(CO)=C1c1ccc(OCc2c(Cl)cccc2Cl)cc1]

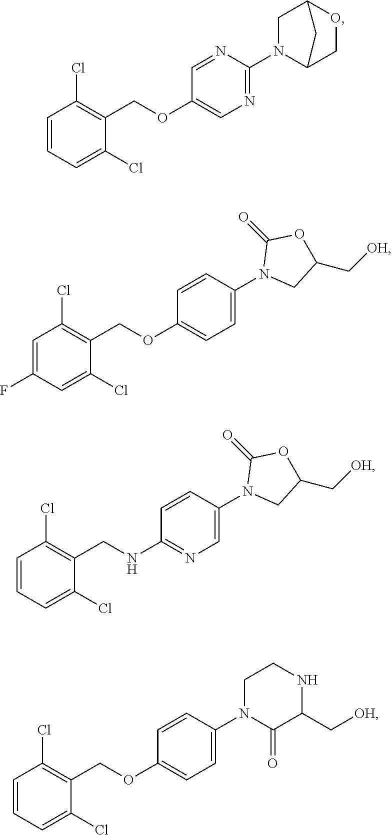 [SMILES: Clc1cccc(Cl)c1COc1cnc(N2CC3CC2CO3)nc1 ; O=C1OC(CO)CN1c1ccc(OCc2c(Cl)cc(F)cc2Cl)cc1 ; O=C1OC(CO)CN1c1ccc(NCc2c(Cl)cccc2Cl)nc1 ; O=C1C(CO)NCCN1c1ccc(OCc2c(Cl)cccc2Cl)cc1]

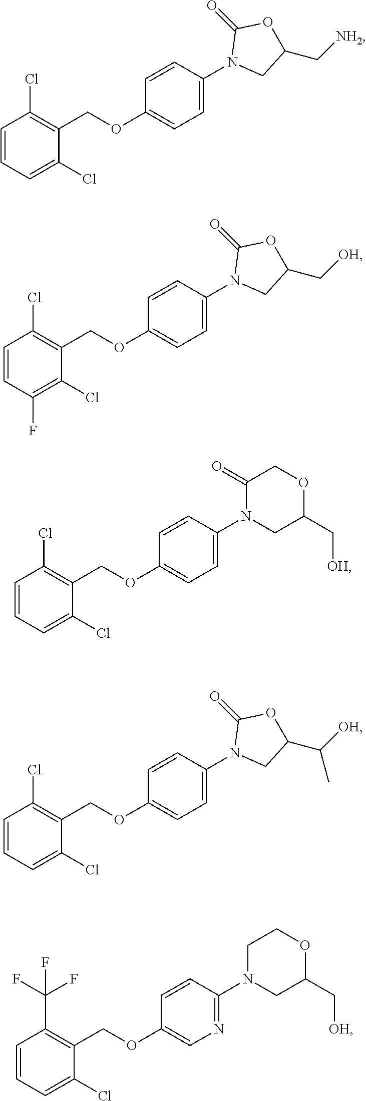 [SMILES: NCC1CN(c2ccc(OCc3c(Cl)cccc3Cl)cc2)C(=O)O1 ; O=C1OC(CO)CN1c1ccc(OCc2c(Cl)ccc(F)c2Cl)cc1 ; O=C1COC(CO)CN1c1ccc(OCc2c(Cl)cccc2Cl)cc1 ; CC(O)C1CN(c2ccc(OCc3c(Cl)cccc3Cl)cc2)C(=O)O1 ; OCC1CN(c2ccc(OCc3c(Cl)cccc3C(F)(F)F)cn2)CCO1]

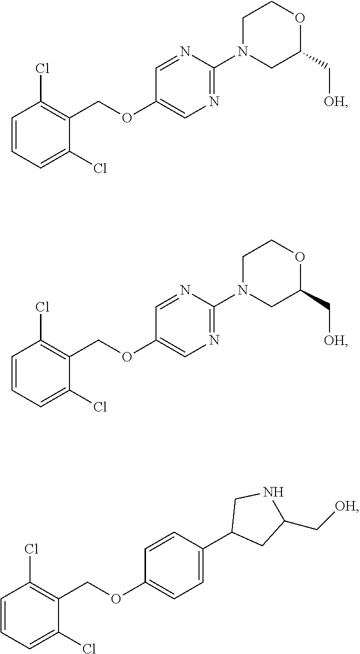 [SMILES: OC[C@@H]1CN(c2ncc(OCc3c(Cl)cccc3Cl)cn2)CCO1 ; OC[C@H]1CN(c2ncc(OCc3c(Cl)cccc3Cl)cn2)CCO1 ; OCC1CC(c2ccc(OCc3c(Cl)cccc3Cl)cc2)CN1]

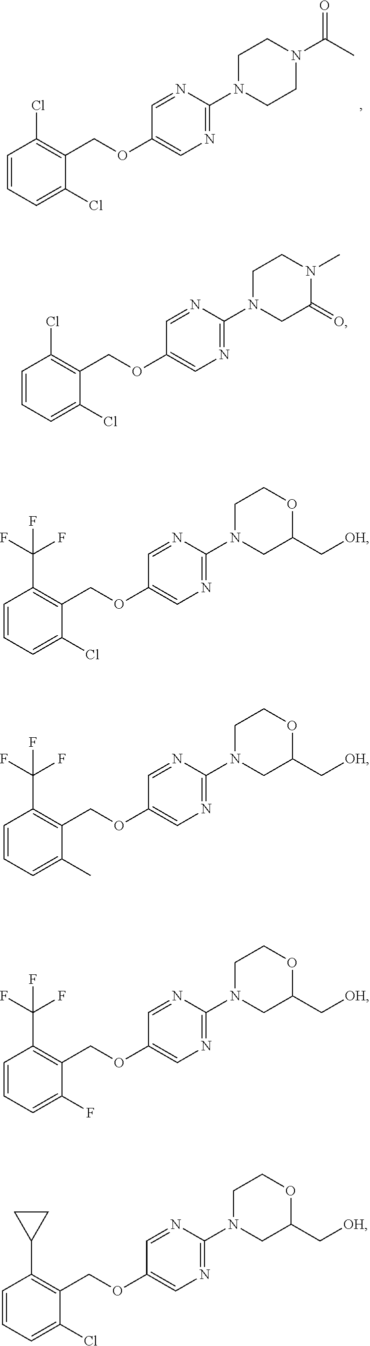 [SMILES: CC(=O)N1CCN(c2ncc(OCc3c(Cl)cccc3Cl)cn2)CC1 ; CN1CCN(c2ncc(OCc3c(Cl)cccc3Cl)cn2)CC1=O ; OCC1CN(c2ncc(OCc3c(Cl)cccc3C(F)(F)F)cn2)CCO1 ; Cc1cccc(C(F)(F)F)c1COc1cnc(N2CCOC(CO)C2)nc1 ; OCC1CN(c2ncc(OCc3c(F)cccc3C(F)(F)F)cn2)CCO1 ; OCC1CN(c2ncc(OCc3c(Cl)cccc3C3CC3)cn2)CCO1]

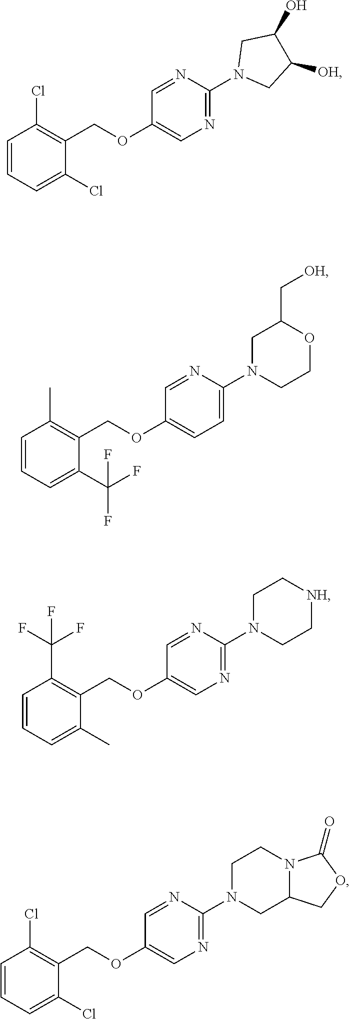 [SMILES: O[C@@H]1CN(c2ncc(OCc3c(Cl)cccc3Cl)cn2)C[C@@H]1O ; Cc1cccc(C(F)(F)F)c1COc1ccc(N2CCOC(CO)C2)nc1 ; Cc1cccc(C(F)(F)F)c1COc1cnc(N2CCNCC2)nc1 ; O=C1OCC2CN(c3ncc(OCc4c(Cl)cccc4Cl)cn3)CCN12]

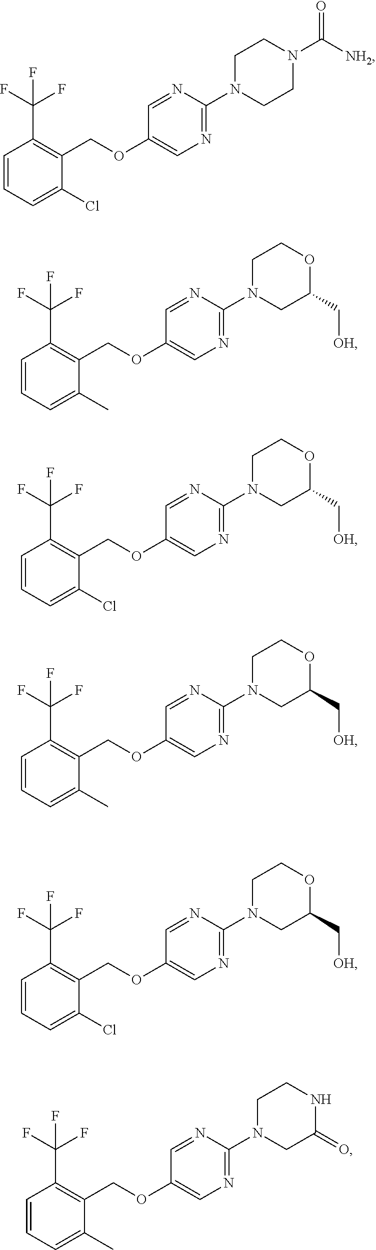 [SMILES: NC(=O)N1CCN(c2ncc(OCc3c(Cl)cccc3C(F)(F)F)cn2)CC1 ; Cc1cccc(C(F)(F)F)c1COc1cnc(N2CCO[C@H](CO)C2)nc1 ; OC[C@@H]1CN(c2ncc(OCc3c(Cl)cccc3C(F)(F)F)cn2)CCO1 ; Cc1cccc(C(F)(F)F)c1COc1cnc(N2CCO[C@@H](CO)C2)nc1 ; OC[C@H]1CN(c2ncc(OCc3c(Cl)cccc3C(F)(F)F)cn2)CCO1 ; Cc1cccc(C(F)(F)F)c1COc1cnc(N2CCNC(=O)C2)nc1]

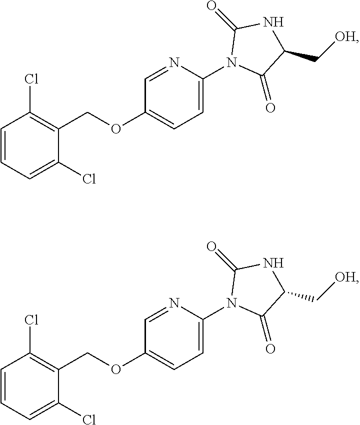 [SMILES: O=C1N[C@@H](CO)C(=O)N1c1ccc(OCc2c(Cl)cccc2Cl)cn1 ; O=C1N[C@H](CO)C(=O)N1c1ccc(OCc2c(Cl)cccc2Cl)cn1]

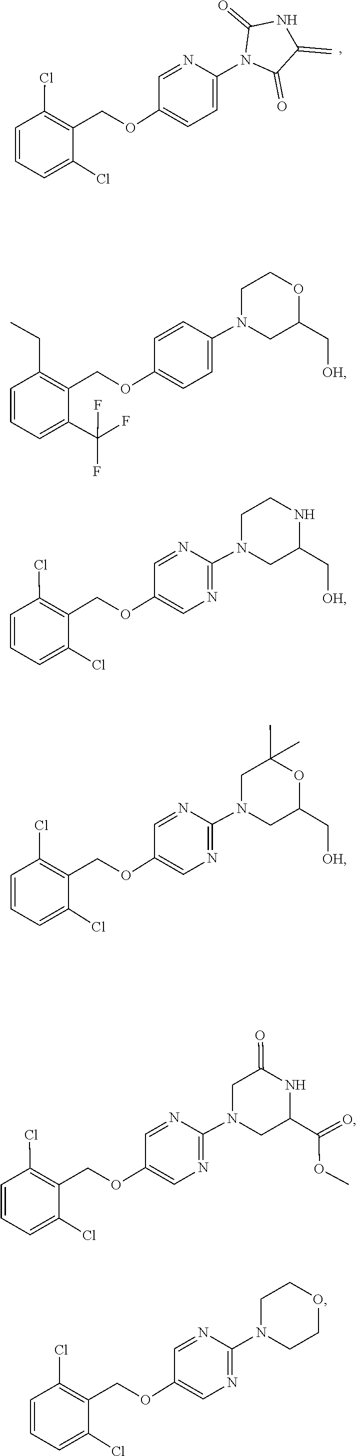

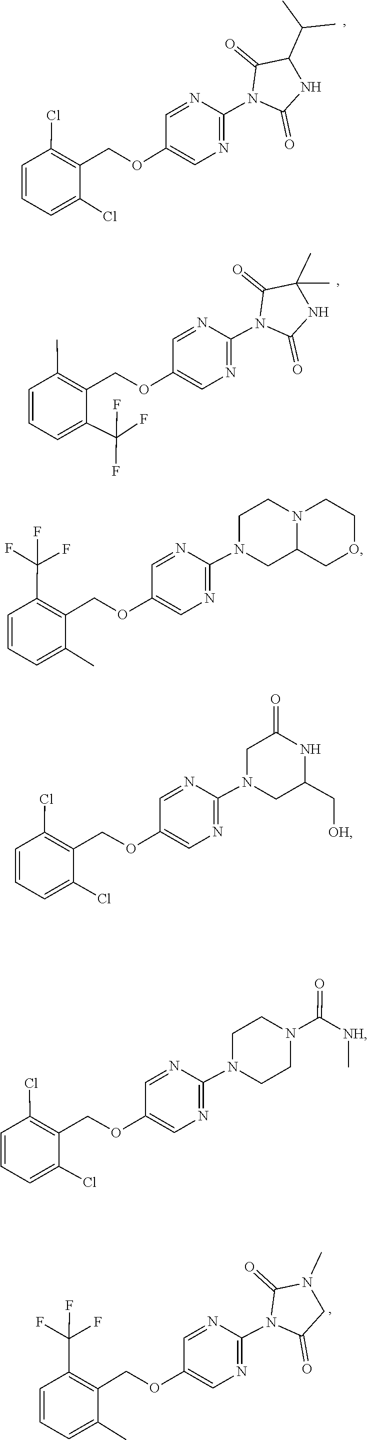 [SMILES: CC(C)C1NC(=O)N(c2ncc(OCc3c(Cl)cccc3Cl)cn2)C1=O ; Cc1cccc(C(F)(F)F)c1COc1cnc(N2C(=O)NC(C)(C)C2=O)nc1 ; Cc1cccc(C(F)(F)F)c1COc1cnc(N2CCN3CCOCC3C2)nc1 ; O=C1CN(c2ncc(OCc3c(Cl)cccc3Cl)cn2)CC(CO)N1 ; CNC(=O)N1CCN(c2ncc(OCc3c(Cl)cccc3Cl)cn2)CC1 ; Cc1cccc(C(F)(F)F)c1COc1cnc(N2C(=O)CN(C)C2=O)nc1]

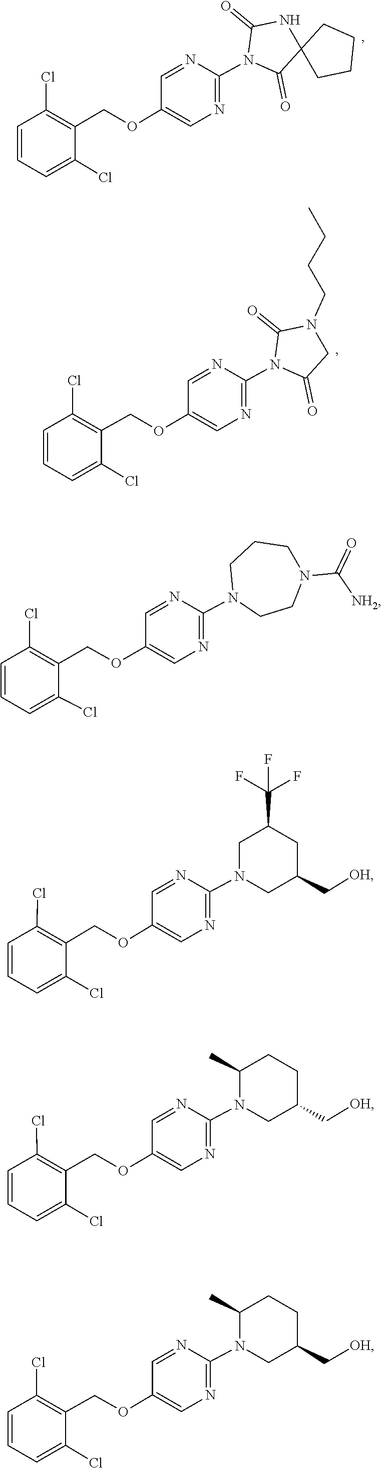 [SMILES: O=C1NC2(CCCC2)C(=O)N1c1ncc(OCc2c(Cl)cccc2Cl)cn1 ; CCCCN1CC(=O)N(c2ncc(OCc3c(Cl)cccc3Cl)cn2)C1=O ; NC(=O)N1CCCN(c2ncc(OCc3c(Cl)cccc3Cl)cn2)CC1 ; OC[C@@H]1C[C@H](C(F)(F)F)CN(c2ncc(OCc3c(Cl)cccc3Cl)cn2)C1 ; C[C@H]1CC[C@H](CO)CN1c1ncc(OCc2c(Cl)cccc2Cl)cn1 ; C[C@H]1CC[C@@H](CO)CN1c1ncc(OCc2c(Cl)cccc2Cl)cn1]

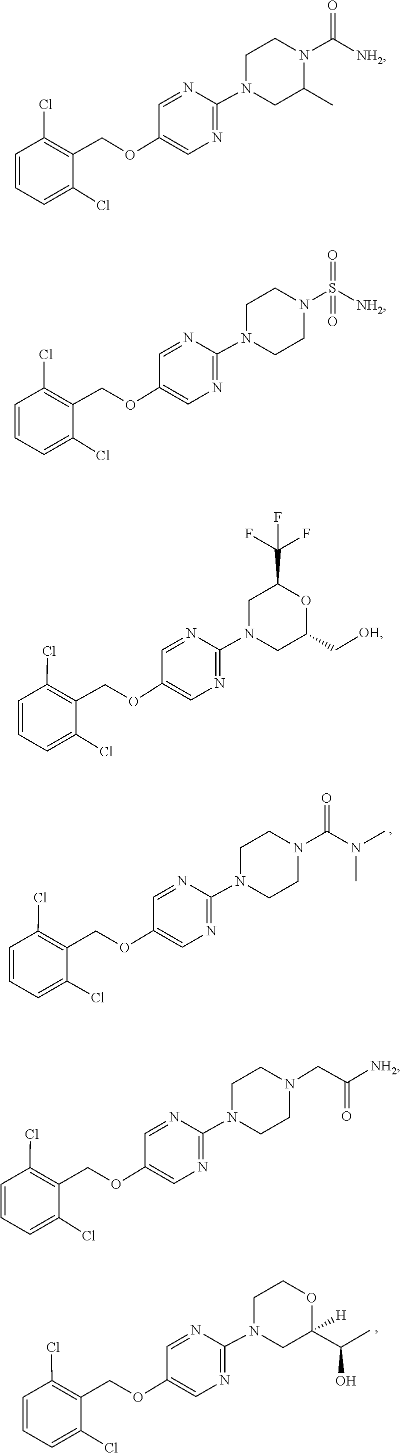 [SMILES: CC1CN(c2ncc(OCc3c(Cl)cccc3Cl)cn2)CCN1C(N)=O ; NS(=O)(=O)N1CCN(c2ncc(OCc3c(Cl)cccc3Cl)cn2)CC1 ; OC[C@@H]1CN(c2ncc(OCc3c(Cl)cccc3Cl)cn2)C[C@@H](C(F)(F)F)O1 ; CN(C)C(=O)N1CCN(c2ncc(OCc3c(Cl)cccc3Cl)cn2)CC1 ; NC(=O)CN1CCN(c2ncc(OCc3c(Cl)cccc3Cl)cn2)CC1 ; C[C@@H](O)[C@H]1CN(c2ncc(OCc3c(Cl)cccc3Cl)cn2)CCO1]

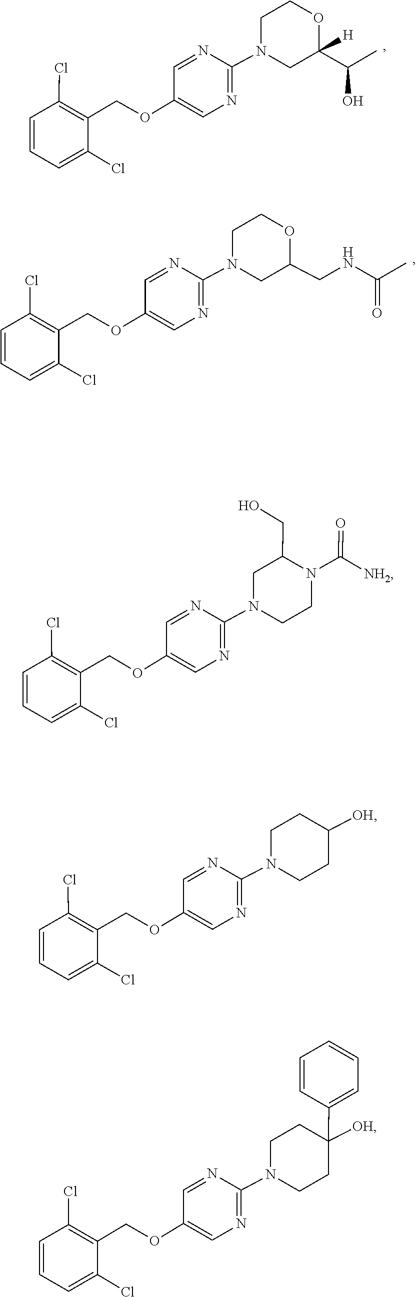 [SMILES: C[C@@H](O)[C@@H]1CN(c2ncc(OCc3c(Cl)cccc3Cl)cn2)CCO1 ; CC(=O)NCC1CN(c2ncc(OCc3c(Cl)cccc3Cl)cn2)CCO1 ; NC(=O)N1CCN(c2ncc(OCc3c(Cl)cccc3Cl)cn2)CC1CO ; OC1CCN(c2ncc(OCc3c(Cl)cccc3Cl)cn2)CC1 ; OC1(c2ccccc2)CCN(c2ncc(OCc3c(Cl)cccc3Cl)cn2)CC1]

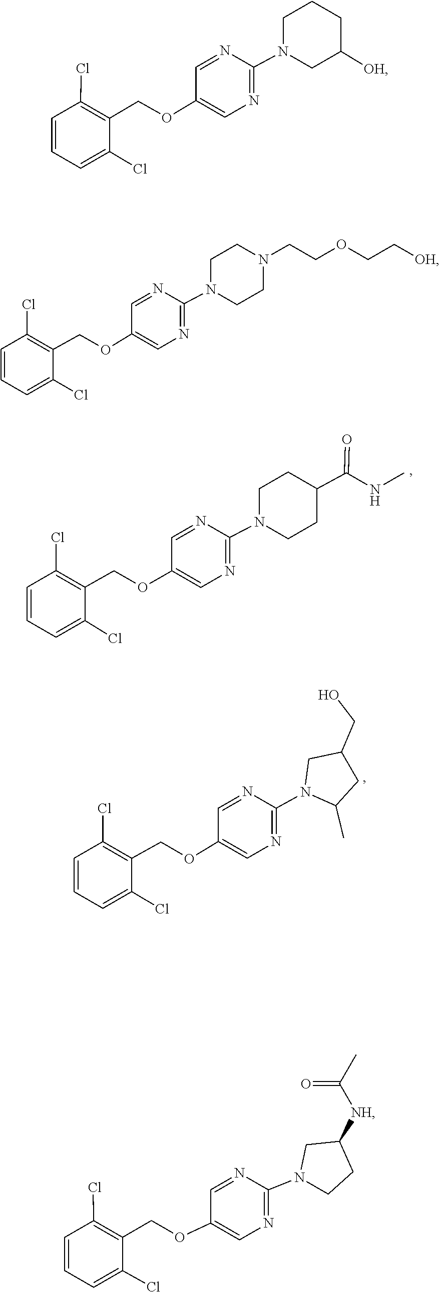 [SMILES: OC1CCCN(c2ncc(OCc3c(Cl)cccc3Cl)cn2)C1 ; OCCOCCN1CCN(c2ncc(OCc3c(Cl)cccc3Cl)cn2)CC1 ; CNC(=O)C1CCN(c2ncc(OCc3c(Cl)cccc3Cl)cn2)CC1 ; CC1CC(CO)CN1c1ncc(OCc2c(Cl)cccc2Cl)cn1 ; CC(=O)N[C@H]1CCN(c2ncc(OCc3c(Cl)cccc3Cl)cn2)C1]

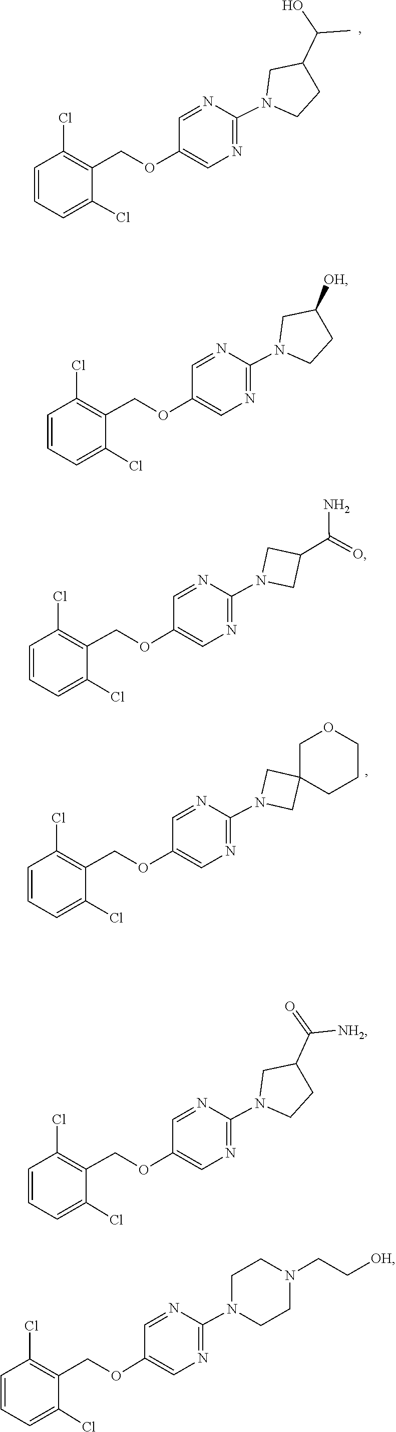 [SMILES: CC(O)C1CCN(c2ncc(OCc3c(Cl)cccc3Cl)cn2)C1 ; O[C@H]1CCN(c2ncc(OCc3c(Cl)cccc3Cl)cn2)C1 ; NC(=O)C1CN(c2ncc(OCc3c(Cl)cccc3Cl)cn2)C1 ; Clc1cccc(Cl)c1COc1cnc(N2CC3(CCCOC3)C2)nc1 ; NC(=O)C1CCN(c2ncc(OCc3c(Cl)cccc3Cl)cn2)C1 ; OCCN1CCN(c2ncc(OCc3c(Cl)cccc3Cl)cn2)CC1]

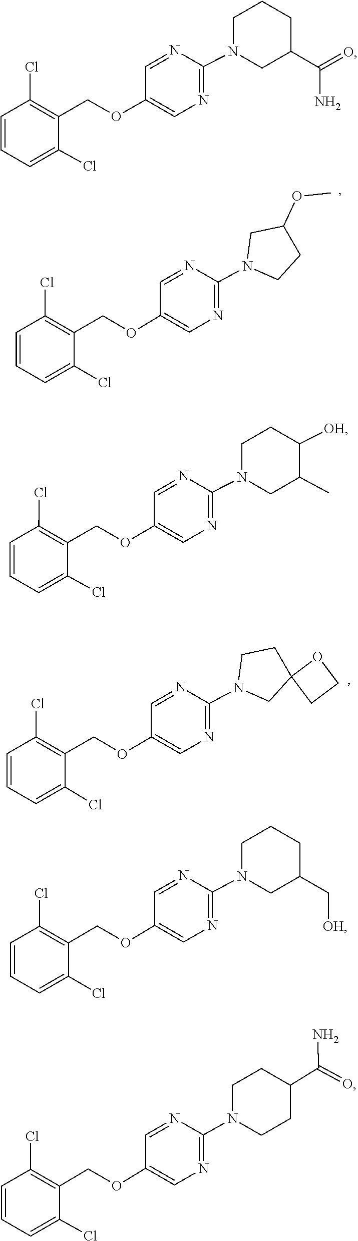 [SMILES: NC(=O)C1CCCN(c2ncc(OCc3c(Cl)cccc3Cl)cn2)C1 ; COC1CCN(c2ncc(OCc3c(Cl)cccc3Cl)cn2)C1 ; CC1CN(c2ncc(OCc3c(Cl)cccc3Cl)cn2)CCC1O ; Clc1cccc(Cl)c1COc1cnc(N2CCC3(CCO3)C2)nc1 ; OCC1CCCN(c2ncc(OCc3c(Cl)cccc3Cl)cn2)C1 ; NC(=O)C1CCN(c2ncc(OCc3c(Cl)cccc3Cl)cn2)CC1]

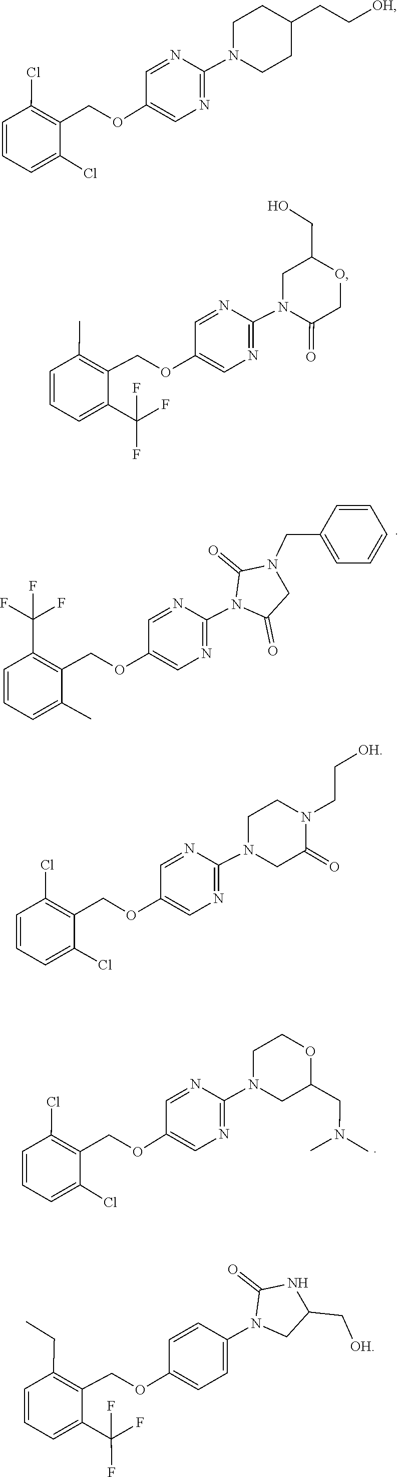 [SMILES: OCCC1CCN(c2ncc(OCc3c(Cl)cccc3Cl)cn2)CC1 ; Cc1cccc(C(F)(F)F)c1COc1cnc(N2CC(CO)OCC2=O)nc1 ; Cc1cccc(C(F)(F)F)c1COc1cnc(N2C(=O)CN(Cc3ccccc3)C2=O)nc1 ; O=C1CN(c2ncc(OCc3c(Cl)cccc3Cl)cn2)CCN1CCO ; CN(C)CC1CN(c2ncc(OCc3c(Cl)cccc3Cl)cn2)CCO1 ; CCc1cccc(C(F)(F)F)c1COc1ccc(N2CC(CO)NC2=O)cc1]

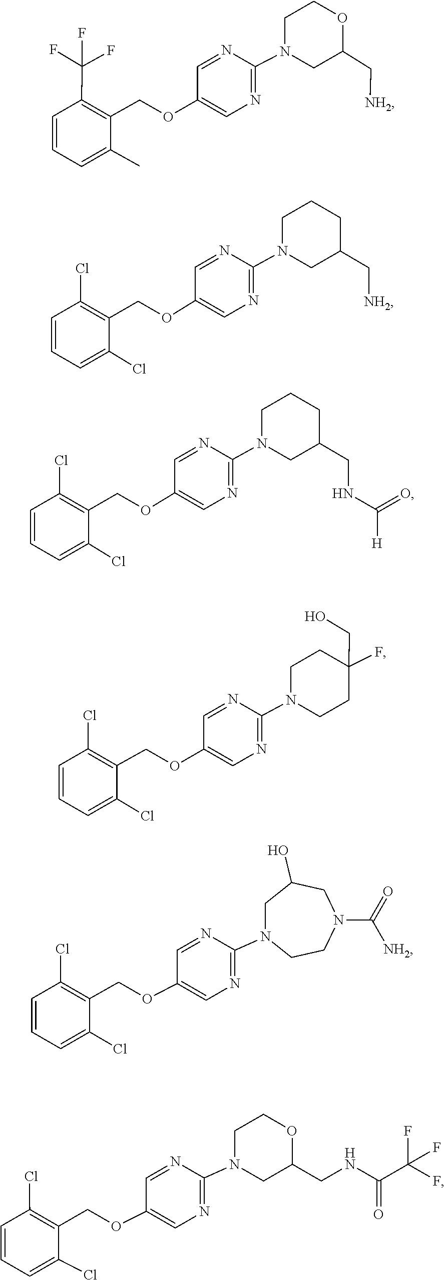 [SMILES: Cc1cccc(C(F)(F)F)c1COc1cnc(N2CCOC(CN)C2)nc1 ; NCC1CCCN(c2ncc(OCc3c(Cl)cccc3Cl)cn2)C1 ; O=CNCC1CCCN(c2ncc(OCc3c(Cl)cccc3Cl)cn2)C1 ; OCC1(F)CCN(c2ncc(OCc3c(Cl)cccc3Cl)cn2)CC1 ; NC(=O)N1CCN(c2ncc(OCc3c(Cl)cccc3Cl)cn2)CC(O)C1 ; O=C(NCC1CN(c2ncc(OCc3c(Cl)cccc3Cl)cn2)CCO1)C(F)(F)F]

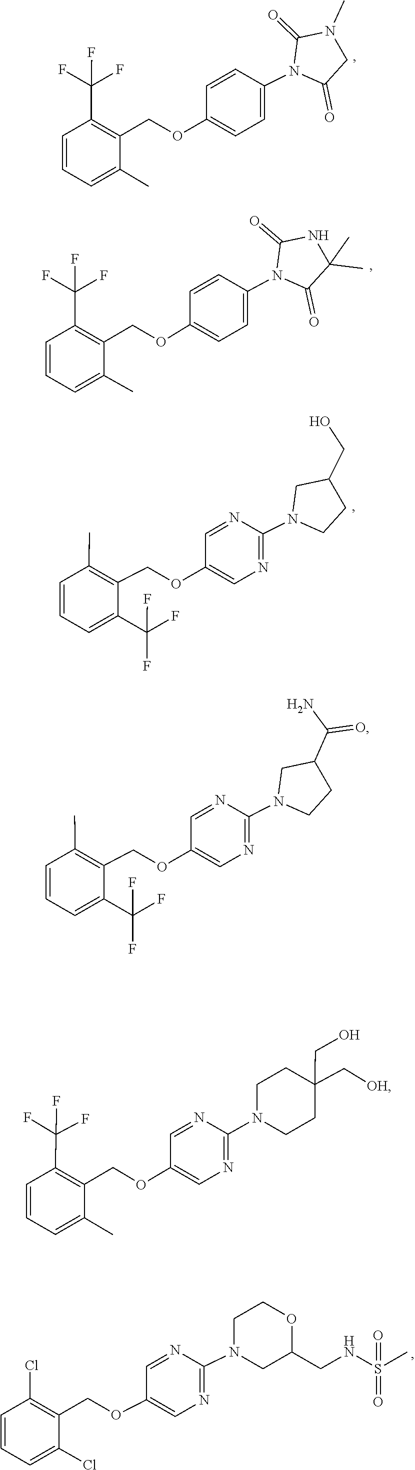 [SMILES: Cc1cccc(C(F)(F)F)c1COc1ccc(N2C(=O)CN(C)C2=O)cc1 ; Cc1cccc(C(F)(F)F)c1COc1ccc(N2C(=O)NC(C)(C)C2=O)cc1 ; Cc1cccc(C(F)(F)F)c1COc1cnc(N2CCC(CO)C2)nc1 ; Cc1cccc(C(F)(F)F)c1COc1cnc(N2CCC(C(N)=O)C2)nc1 ; Cc1cccc(C(F)(F)F)c1COc1cnc(N2CCC(CO)(CO)CC2)nc1 ; CS(=O)(=O)NCC1CN(c2ncc(OCc3c(Cl)cccc3Cl)cn2)CCO1]

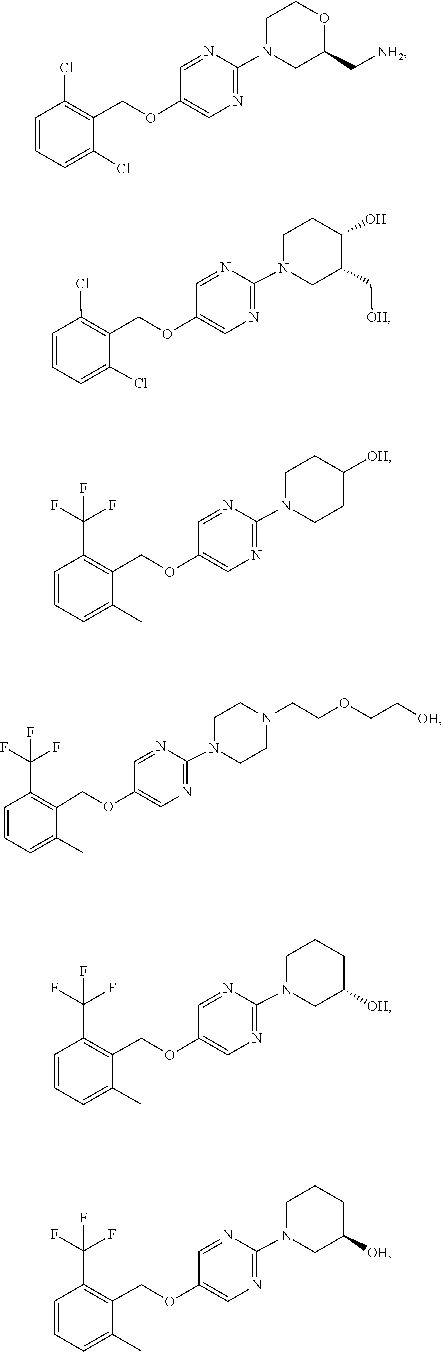 [SMILES: NC[C@H]1CN(c2ncc(OCc3c(Cl)cccc3Cl)cn2)CCO1 ; OC[C@@H]1CN(c2ncc(OCc3c(Cl)cccc3Cl)cn2)CC[C@@H]1O ; Cc1cccc(C(F)(F)F)c1COc1cnc(N2CCC(O)CC2)nc1 ; Cc1cccc(C(F)(F)F)c1COc1cnc(N2CCN(CCOCCO)CC2)nc1 ; Cc1cccc(C(F)(F)F)c1COc1cnc(N2CCC[C@H](O)C2)nc1 ; Cc1cccc(C(F)(F)F)c1COc1cnc(N2CCC[C@@H](O)C2)nc1]

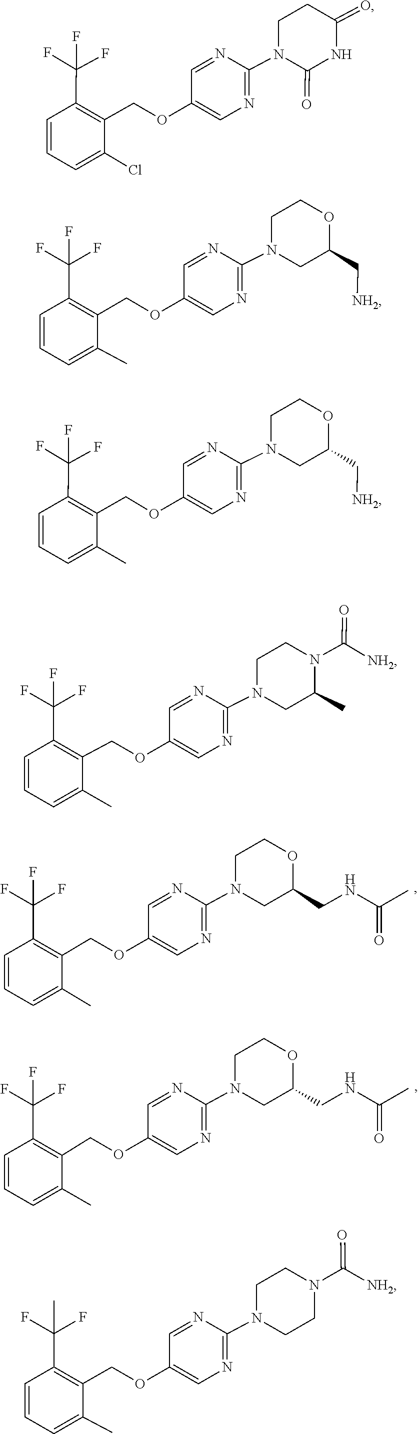

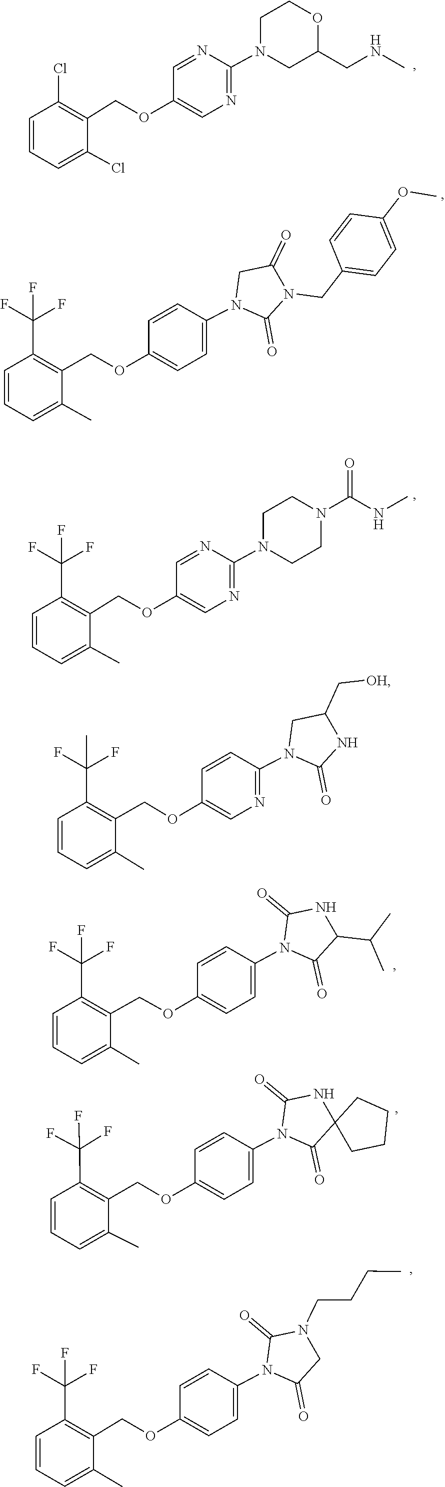

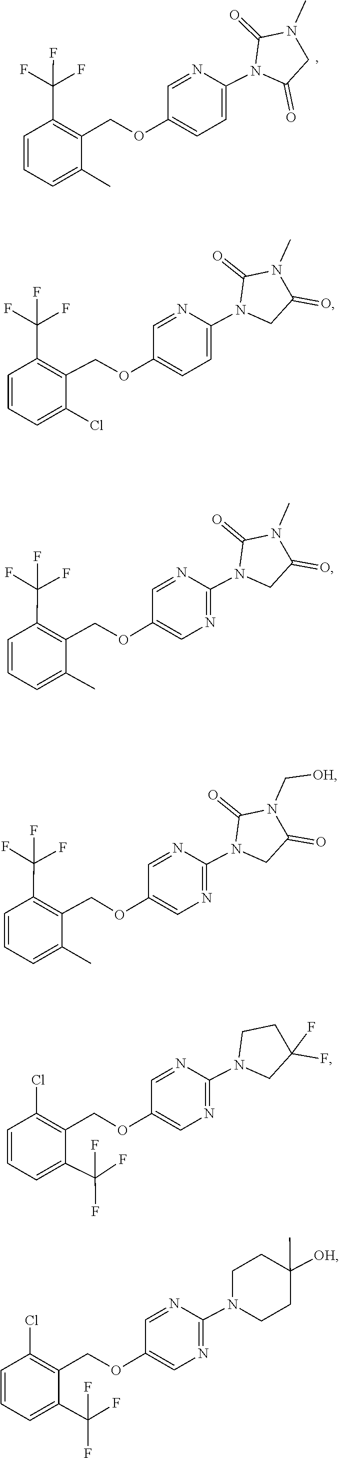 [SMILES: Cc1cccc(C(F)(F)F)c1COc1ccc(N2C(=O)CN(C)C2=O)nc1 ; CN1C(=O)CN(c2ccc(OCc3c(Cl)cccc3C(F)(F)F)cn2)C1=O ; Cc1cccc(C(F)(F)F)c1COc1cnc(N2CC(=O)N(C)C2=O)nc1 ; Cc1cccc(C(F)(F)F)c1COc1cnc(N2CC(=O)N(CO)C2=O)nc1 ; FC1(F)CCN(c2ncc(OCc3c(Cl)cccc3C(F)(F)F)cn2)C1 ; CC1(O)CCN(c2ncc(OCc3c(Cl)cccc3C(F)(F)F)cn2)CC1]

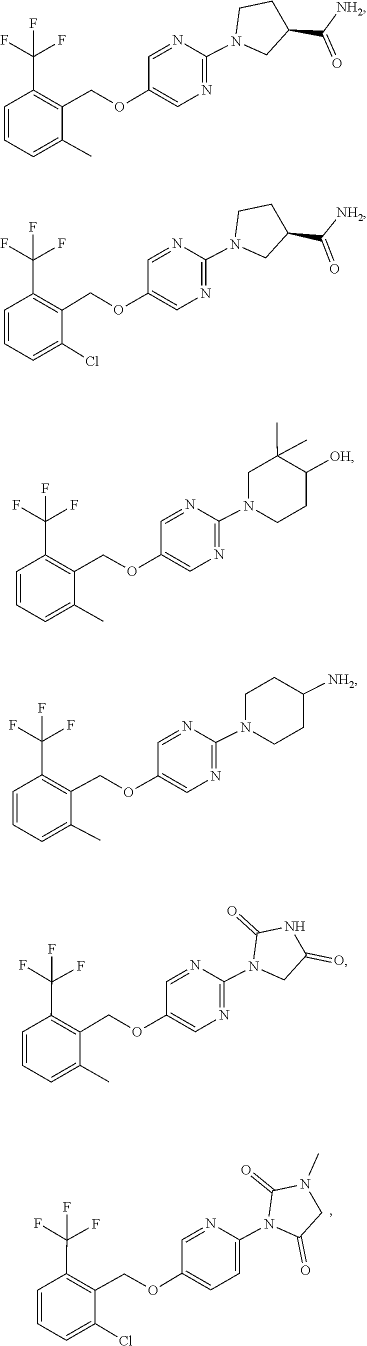 [SMILES: Cc1cccc(C(F)(F)F)c1COc1cnc(N2CC[C@@H](C(N)=O)C2)nc1 ; NC(=O)[C@@H]1CCN(c2ncc(OCc3c(Cl)cccc3C(F)(F)F)cn2)C1 ; Cc1cccc(C(F)(F)F)c1COc1cnc(N2CCC(O)C(C)(C)C2)nc1 ; Cc1cccc(C(F)(F)F)c1COc1cnc(N2CCC(N)CC2)nc1 ; Cc1cccc(C(F)(F)F)c1COc1cnc(N2CC(=O)NC2=O)nc1 ; CN1CC(=O)N(c2ccc(OCc3c(Cl)cccc3C(F)(F)F)cn2)C1=O]

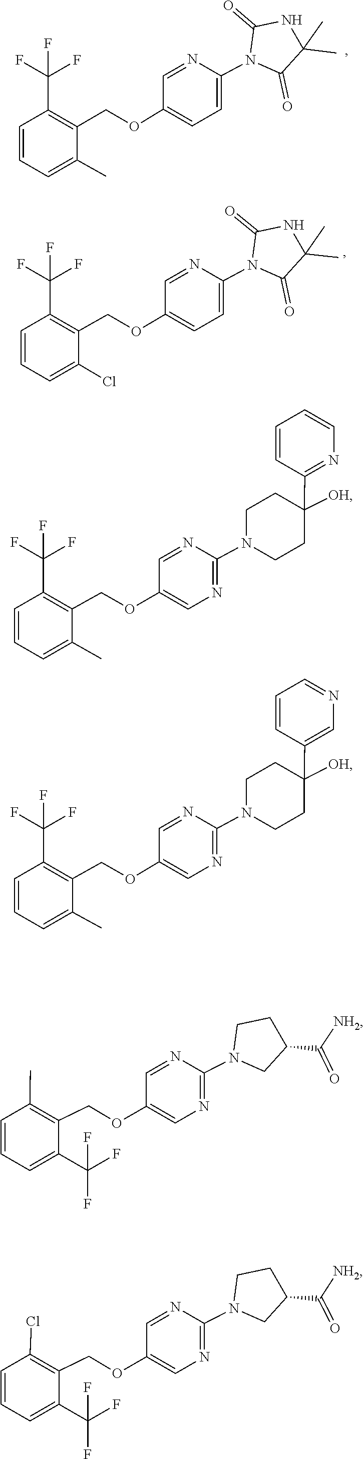 [SMILES: Cc1cccc(C(F)(F)F)c1COc1ccc(N2C(=O)NC(C)(C)C2=O)nc1 ; CC1(C)NC(=O)N(c2ccc(OCc3c(Cl)cccc3C(F)(F)F)cn2)C1=O ; Cc1cccc(C(F)(F)F)c1COc1cnc(N2CCC(O)(c3ccccn3)CC2)nc1 ; Cc1cccc(C(F)(F)F)c1COc1cnc(N2CCC(O)(c3cccnc3)CC2)nc1 ; Cc1cccc(C(F)(F)F)c1COc1cnc(N2CC[C@H](C(N)=O)C2)nc1 ; NC(=O)[C@H]1CCN(c2ncc(OCc3c(Cl)cccc3C(F)(F)F)cn2)C1]

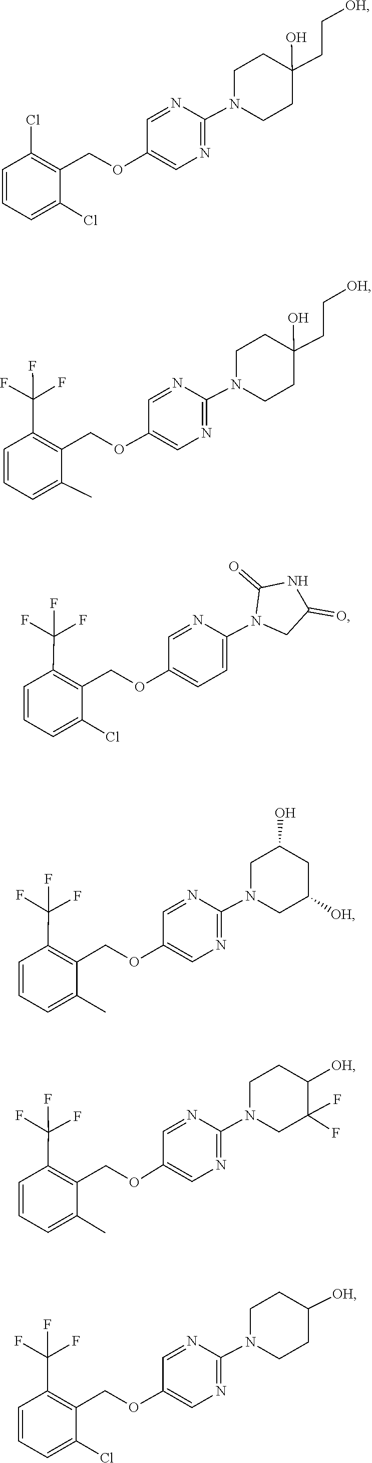 [SMILES: OCCC1(O)CCN(c2ncc(OCc3c(Cl)cccc3Cl)cn2)CC1 ; Cc1cccc(C(F)(F)F)c1COc1cnc(N2CCC(O)(CCO)CC2)nc1 ; O=C1CN(c2ccc(OCc3c(Cl)cccc3C(F)(F)F)cn2)C(=O)N1 ; Cc1cccc(C(F)(F)F)c1COc1cnc(N2C[C@H](O)C[C@H](O)C2)nc1 ; Cc1cccc(C(F)(F)F)c1COc1cnc(N2CCC(O)C(F)(F)C2)nc1 ; OC1CCN(c2ncc(OCc3c(Cl)cccc3C(F)(F)F)cn2)CC1]

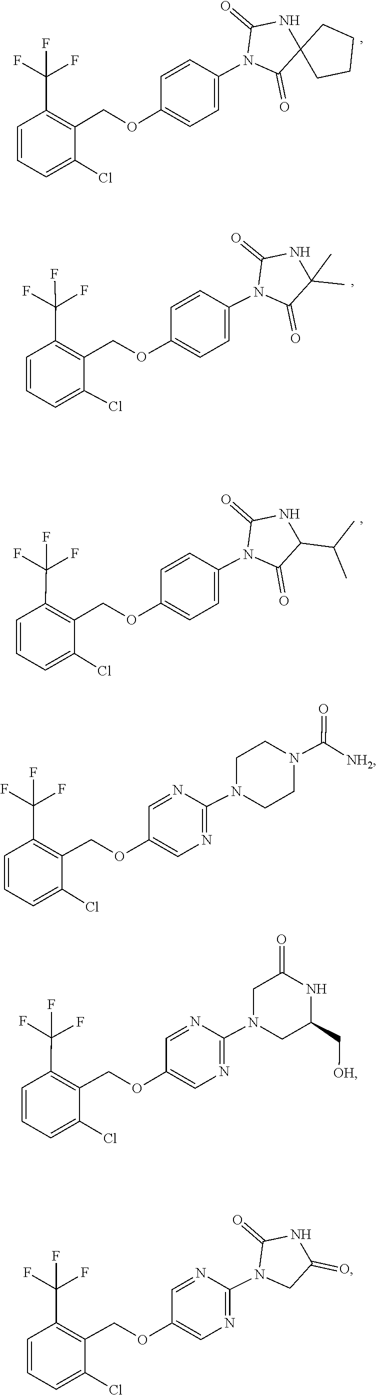 [SMILES: O=C1NC2(CCCC2)C(=O)N1c1ccc(OCc2c(Cl)cccc2C(F)(F)F)cc1 ; CC1(C)NC(=O)N(c2ccc(OCc3c(Cl)cccc3C(F)(F)F)cc2)C1=O ; CC(C)C1NC(=O)N(c2ccc(OCc3c(Cl)cccc3C(F)(F)F)cc2)C1=O ; NC(=O)N1CCN(c2ncc(OCc3c(Cl)cccc3C(F)(F)F)cn2)CC1 ; O=C1CN(c2ncc(OCc3c(Cl)cccc3C(F)(F)F)cn2)C[C@H](CO)N1 ; O=C1CN(c2ncc(OCc3c(Cl)cccc3C(F)(F)F)cn2)C(=O)N1]

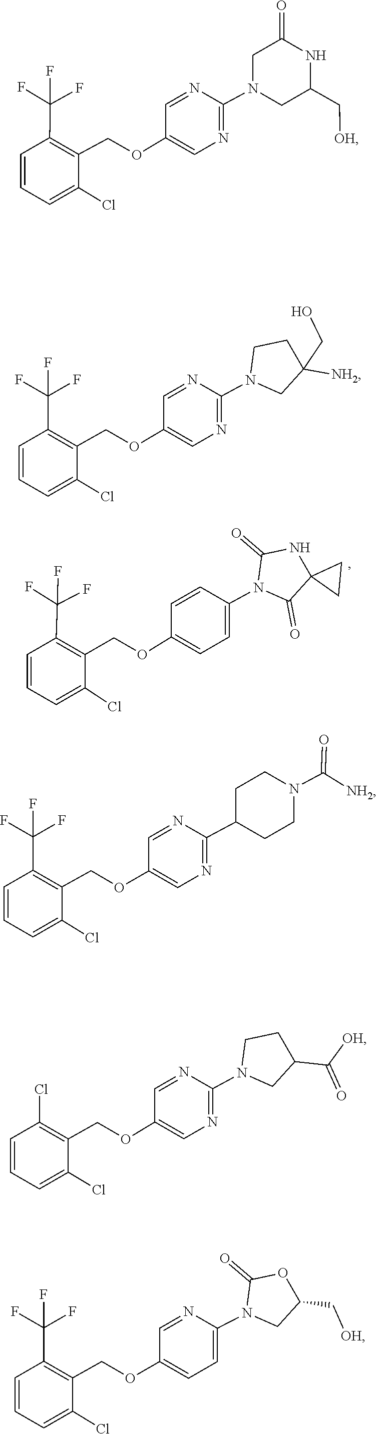 [SMILES: O=C1CN(c2ncc(OCc3c(Cl)cccc3C(F)(F)F)cn2)CC(CO)N1 ; NC1(CO)CCN(c2ncc(OCc3c(Cl)cccc3C(F)(F)F)cn2)C1 ; O=C1NC2(CC2)C(=O)N1c1ccc(OCc2c(Cl)cccc2C(F)(F)F)cc1 ; NC(=O)N1CCC(c2ncc(OCc3c(Cl)cccc3C(F)(F)F)cn2)CC1 ; O=C(O)C1CCN(c2ncc(OCc3c(Cl)cccc3Cl)cn2)C1 ; O=C1O[C@H](CO)CN1c1ccc(OCc2c(Cl)cccc2C(F)(F)F)cn1]

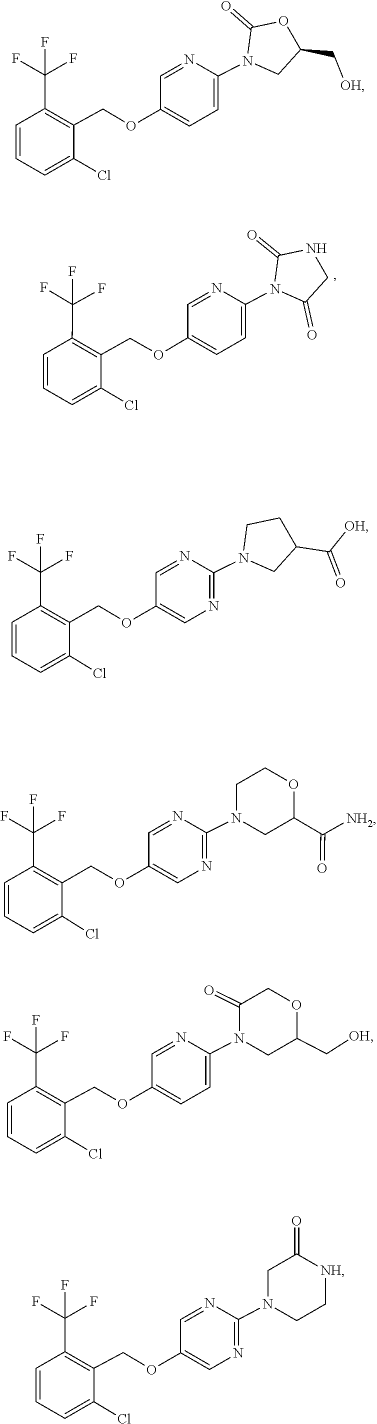 [SMILES: O=C1O[C@@H](CO)CN1c1ccc(OCc2c(Cl)cccc2C(F)(F)F)cn1 ; O=C1CNC(=O)N1c1ccc(OCc2c(Cl)cccc2C(F)(F)F)cn1 ; O=C(O)C1CCN(c2ncc(OCc3c(Cl)cccc3C(F)(F)F)cn2)C1 ; NC(=O)C1CN(c2ncc(OCc3c(Cl)cccc3C(F)(F)F)cn2)CCO1 ; O=C1COC(CO)CN1c1ccc(OCc2c(Cl)cccc2C(F)(F)F)cn1 ; O=C1CN(c2ncc(OCc3c(Cl)cccc3C(F)(F)F)cn2)CCN1]

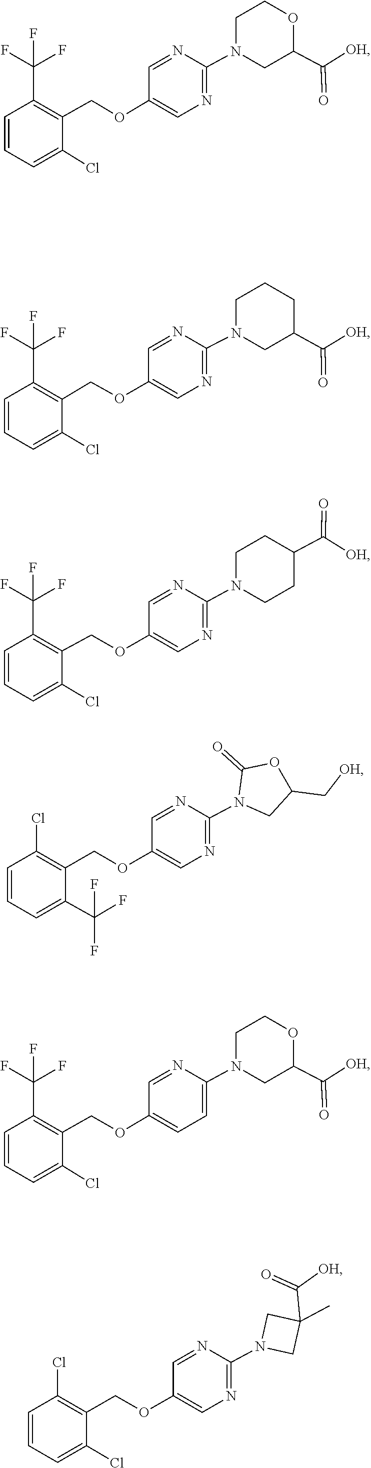 [SMILES: O=C(O)C1CN(c2ncc(OCc3c(Cl)cccc3C(F)(F)F)cn2)CCO1 ; O=C(O)C1CCCN(c2ncc(OCc3c(Cl)cccc3C(F)(F)F)cn2)C1 ; O=C(O)C1CCN(c2ncc(OCc3c(Cl)cccc3C(F)(F)F)cn2)CC1 ; O=C1OC(CO)CN1c1ncc(OCc2c(Cl)cccc2C(F)(F)F)cn1 ; O=C(O)C1CN(c2ccc(OCc3c(Cl)cccc3C(F)(F)F)cn2)CCO1 ; CC1(C(=O)O)CN(c2ncc(OCc3c(Cl)cccc3Cl)cn2)C1]

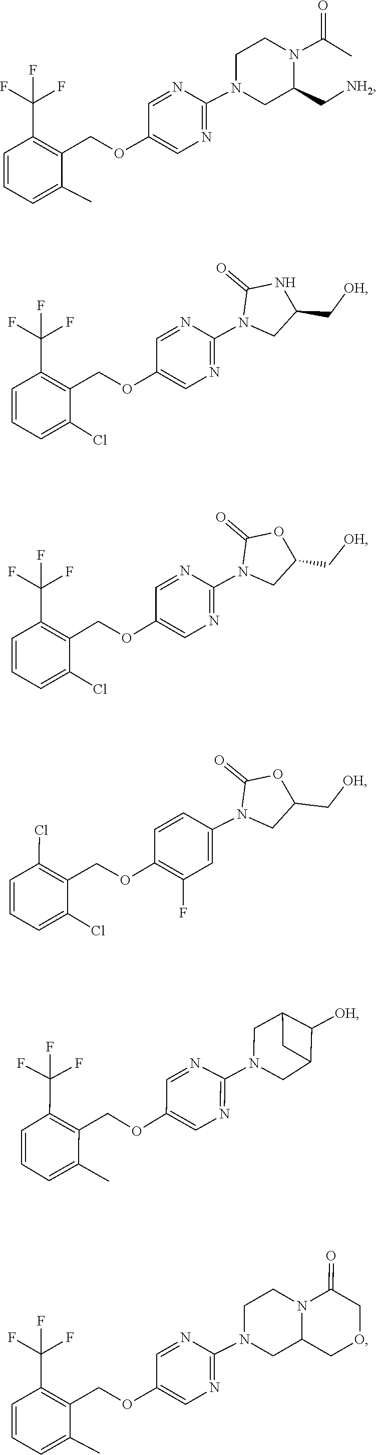 [SMILES: CC(=O)N1CCN(c2ncc(OCc3c(C)cccc3C(F)(F)F)cn2)C[C@@H]1CN ; O=C1N[C@@H](CO)CN1c1ncc(OCc2c(Cl)cccc2C(F)(F)F)cn1 ; O=C1O[C@H](CO)CN1c1ncc(OCc2c(Cl)cccc2C(F)(F)F)cn1 ; O=C1OC(CO)CN1c1ccc(OCc2c(Cl)cccc2Cl)c(F)c1 ; Cc1cccc(C(F)(F)F)c1COc1cnc(N2CC3CC(C2)C3O)nc1 ; Cc1cccc(C(F)(F)F)c1COc1cnc(N2CCN3C(=O)COCC3C2)nc1]

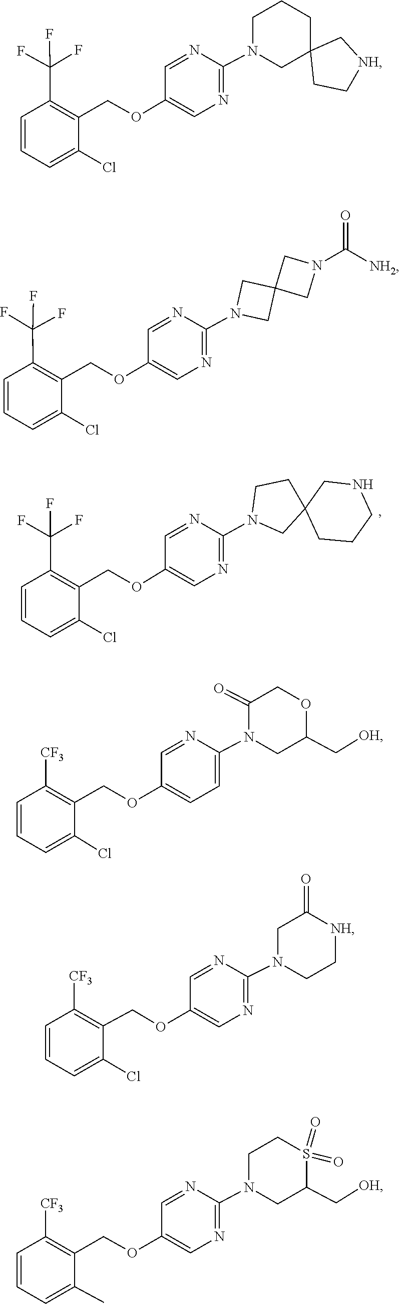 [SMILES: FC(F)(F)c1cccc(Cl)c1COc1cnc(N2CCCC3(CCNC3)C2)nc1 ; NC(=O)N1CC2(C1)CN(c1ncc(OCc3c(Cl)cccc3C(F)(F)F)cn1)C2 ; FC(F)(F)c1cccc(Cl)c1COc1cnc(N2CCC3(CCCNC3)C2)nc1 ; O=C1COC(CO)CN1c1ccc(OCc2c(Cl)cccc2C(F)(F)F)cn1 ; O=C1CN(c2ncc(OCc3c(Cl)cccc3C(F)(F)F)cn2)CCN1 ; Cc1cccc(C(F)(F)F)c1COc1cnc(N2CCS(=O)(=O)C(CO)C2)nc1]

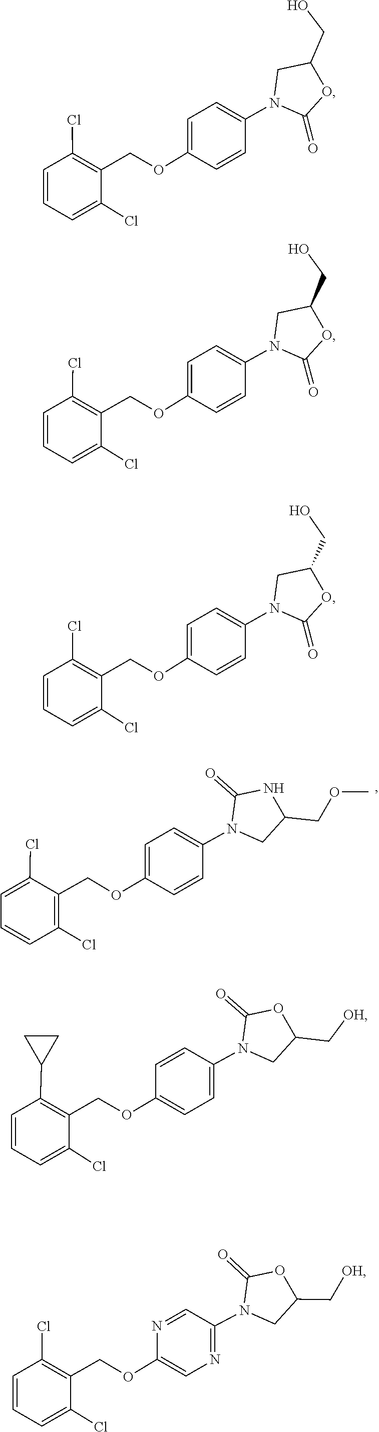 [SMILES: O=C1OC(CO)CN1c1ccc(OCc2c(Cl)cccc2Cl)cc1 ; O=C1O[C@H](CO)CN1c1ccc(OCc2c(Cl)cccc2Cl)cc1 ; O=C1O[C@@H](CO)CN1c1ccc(OCc2c(Cl)cccc2Cl)cc1 ; COCC1CN(c2ccc(OCc3c(Cl)cccc3Cl)cc2)C(=O)N1 ; O=C1OC(CO)CN1c1ccc(OCc2c(Cl)cccc2C2CC2)cc1 ; O=C1OC(CO)CN1c1cnc(OCc2c(Cl)cccc2Cl)cn1]

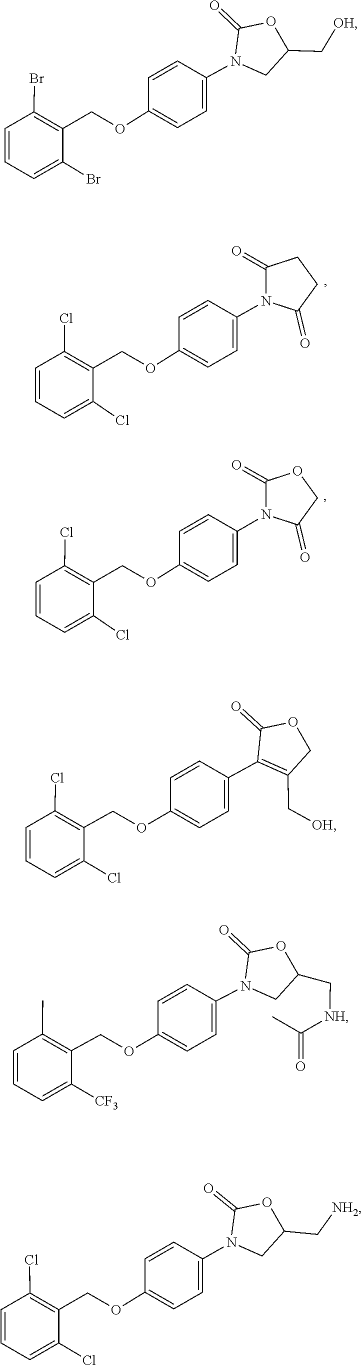 [SMILES: O=C1OC(CO)CN1c1ccc(OCc2c(Br)cccc2Br)cc1 ; O=C1CCC(=O)N1c1ccc(OCc2c(Cl)cccc2Cl)cc1 ; O=C1COC(=O)N1c1ccc(OCc2c(Cl)cccc2Cl)cc1 ; O=C1OCC(CO)=C1c1ccc(OCc2c(Cl)cccc2Cl)cc1 ; CC(=O)NCC1CN(c2ccc(OCc3c(C)cccc3C(F)(F)F)cc2)C(=O)O1 ; NCC1CN(c2ccc(OCc3c(Cl)cccc3Cl)cc2)C(=O)O1]

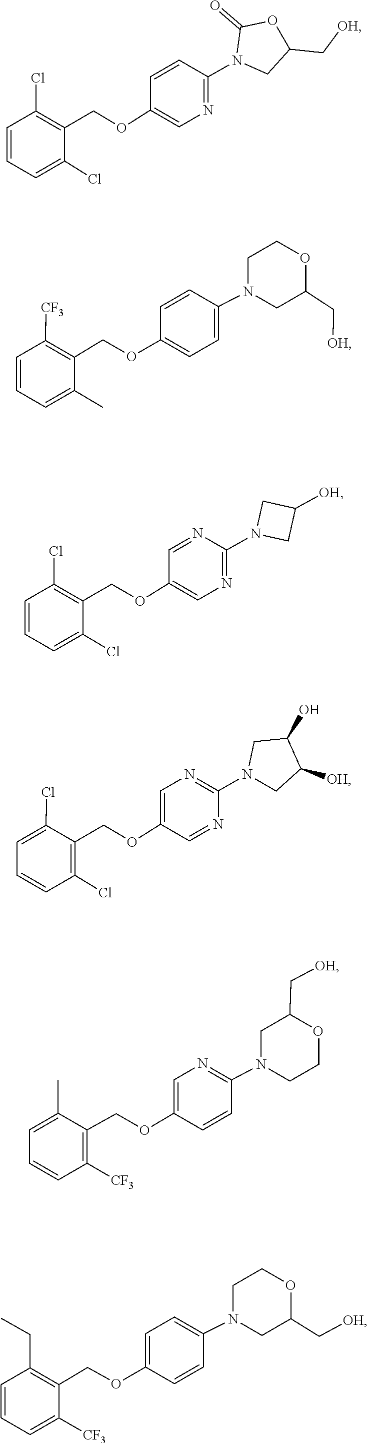 [SMILES: O=C1OC(CO)CN1c1ccc(OCc2c(Cl)cccc2Cl)cn1 ; Cc1cccc(C(F)(F)F)c1COc1ccc(N2CCOC(CO)C2)cc1 ; OC1CN(c2ncc(OCc3c(Cl)cccc3Cl)cn2)C1 ; O[C@@H]1CN(c2ncc(OCc3c(Cl)cccc3Cl)cn2)C[C@@H]1O ; Cc1cccc(C(F)(F)F)c1COc1ccc(N2CCOC(CO)C2)nc1 ; CCc1cccc(C(F)(F)F)c1COc1ccc(N2CCOC(CO)C2)cc1]

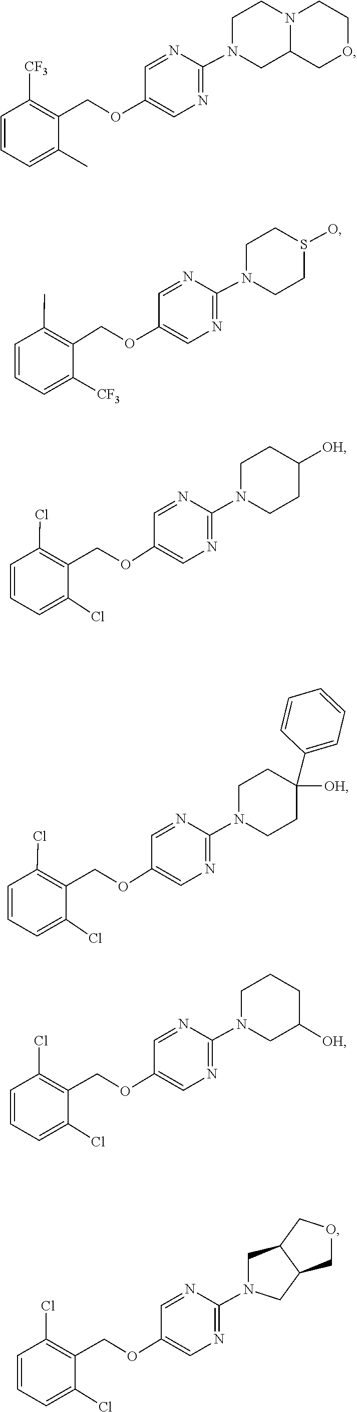

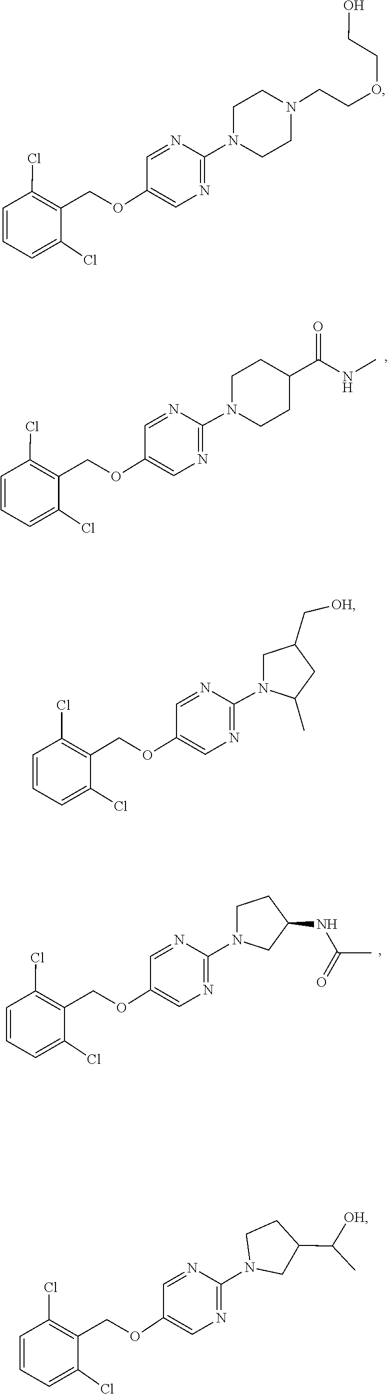 [SMILES: OCCOCCN1CCN(c2ncc(OCc3c(Cl)cccc3Cl)cn2)CC1 ; CNC(=O)C1CCN(c2ncc(OCc3c(Cl)cccc3Cl)cn2)CC1 ; CC1CC(CO)CN1c1ncc(OCc2c(Cl)cccc2Cl)cn1 ; CC(=O)N[C@@H]1CCN(c2ncc(OCc3c(Cl)cccc3Cl)cn2)C1 ; CC(O)C1CCN(c2ncc(OCc3c(Cl)cccc3Cl)cn2)C1]

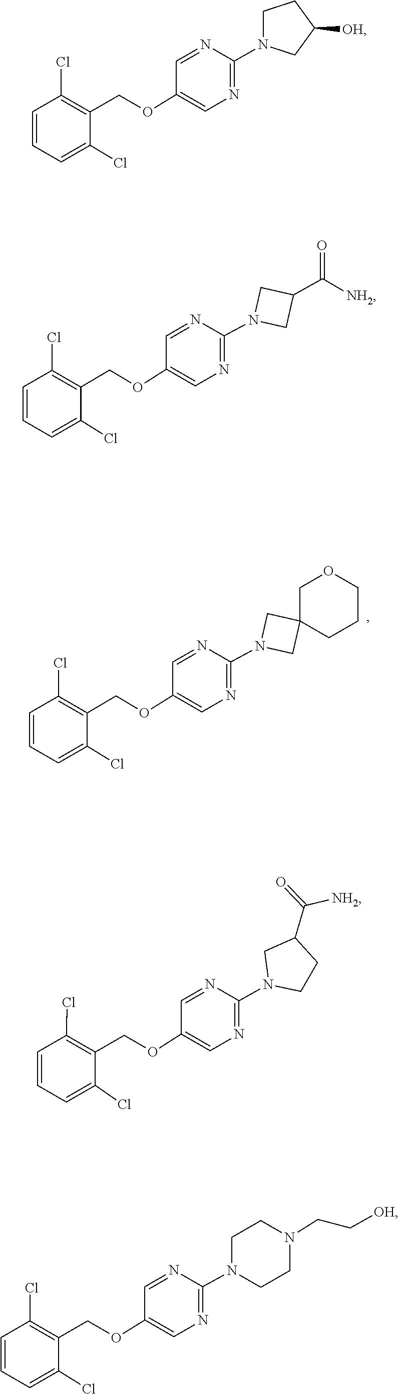 [SMILES: O[C@@H]1CCN(c2ncc(OCc3c(Cl)cccc3Cl)cn2)C1 ; NC(=O)C1CN(c2ncc(OCc3c(Cl)cccc3Cl)cn2)C1 ; Clc1cccc(Cl)c1COc1cnc(N2CC3(CCCOC3)C2)nc1 ; NC(=O)C1CCN(c2ncc(OCc3c(Cl)cccc3Cl)cn2)C1 ; OCCN1CCN(c2ncc(OCc3c(Cl)cccc3Cl)cn2)CC1]

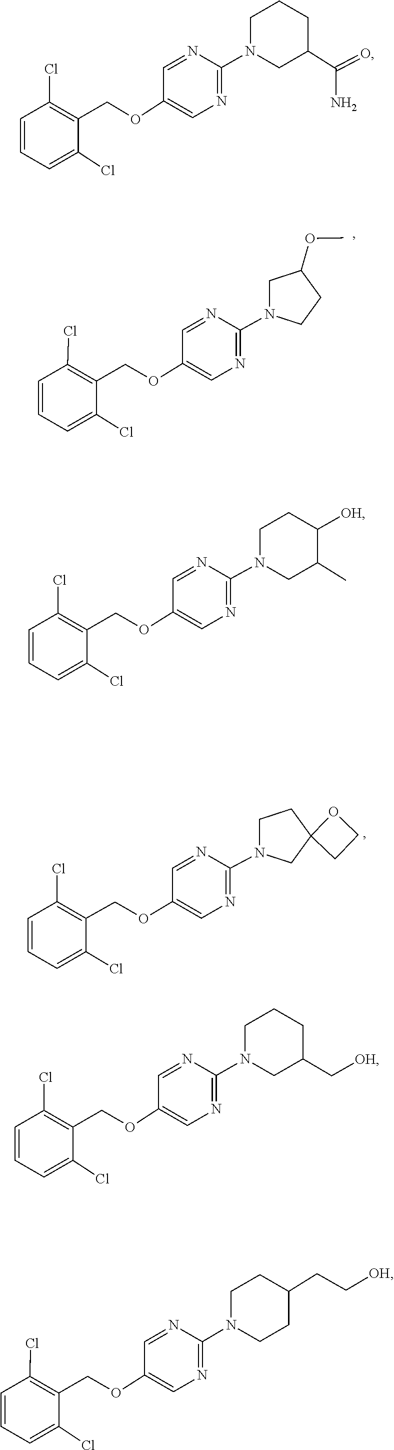 [SMILES: NC(=O)C1CCCN(c2ncc(OCc3c(Cl)cccc3Cl)cn2)C1 ; COC1CCN(c2ncc(OCc3c(Cl)cccc3Cl)cn2)C1 ; CC1CN(c2ncc(OCc3c(Cl)cccc3Cl)cn2)CCC1O ; Clc1cccc(Cl)c1COc1cnc(N2CCC3(CCO3)C2)nc1 ; OCC1CCCN(c2ncc(OCc3c(Cl)cccc3Cl)cn2)C1 ; OCCC1CCN(c2ncc(OCc3c(Cl)cccc3Cl)cn2)CC1]

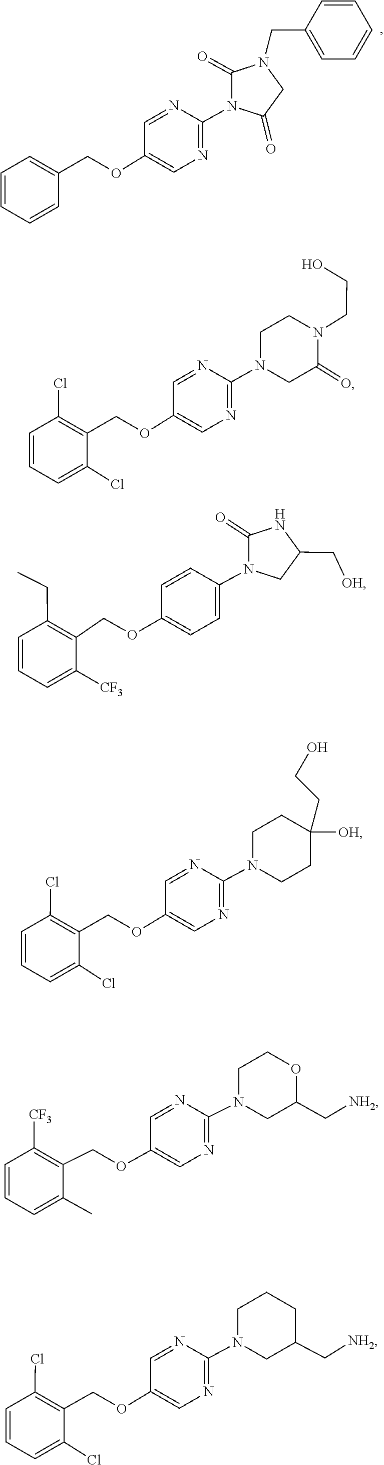 [SMILES: O=C1CN(Cc2ccccc2)C(=O)N1c1ncc(OCc2ccccc2)cn1 ; O=C1CN(c2ncc(OCc3c(Cl)cccc3Cl)cn2)CCN1CCO ; CCc1cccc(C(F)(F)F)c1COc1ccc(N2CC(CO)NC2=O)cc1 ; OCCC1(O)CCN(c2ncc(OCc3c(Cl)cccc3Cl)cn2)CC1 ; Cc1cccc(C(F)(F)F)c1COc1cnc(N2CCOC(CN)C2)nc1 ; NCC1CCCN(c2ncc(OCc3c(Cl)cccc3Cl)cn2)C1]

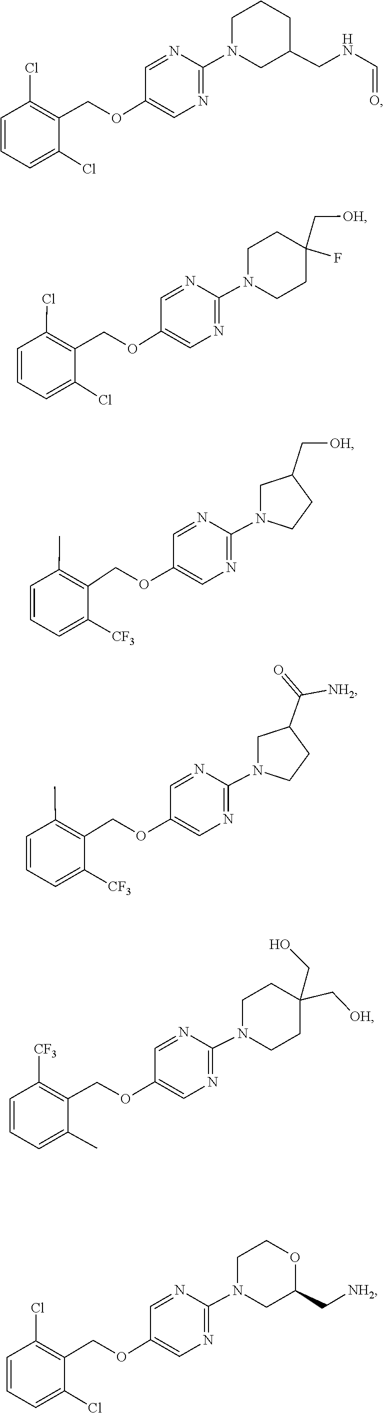 [SMILES: O=CNCC1CCCN(c2ncc(OCc3c(Cl)cccc3Cl)cn2)C1 ; OCC1(F)CCN(c2ncc(OCc3c(Cl)cccc3Cl)cn2)CC1 ; Cc1cccc(C(F)(F)F)c1COc1cnc(N2CCC(CO)C2)nc1 ; Cc1cccc(C(F)(F)F)c1COc1cnc(N2CCC(C(N)=O)C2)nc1 ; Cc1cccc(C(F)(F)F)c1COc1cnc(N2CCC(CO)(CO)CC2)nc1 ; NC[C@H]1CN(c2ncc(OCc3c(Cl)cccc3Cl)cn2)CCO1]

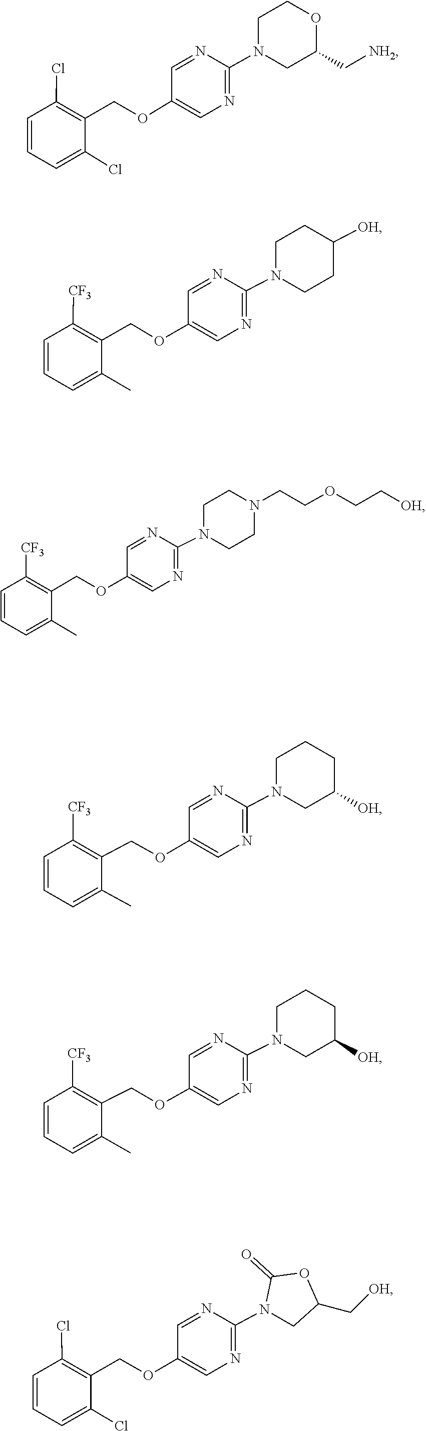 [SMILES: NC[C@@H]1CN(c2ncc(OCc3c(Cl)cccc3Cl)cn2)CCO1 ; Cc1cccc(C(F)(F)F)c1COc1cnc(N2CCC(O)CC2)nc1 ; Cc1cccc(C(F)(F)F)c1COc1cnc(N2CCN(CCOCCO)CC2)nc1 ; Cc1cccc(C(F)(F)F)c1COc1cnc(N2CCC[C@H](O)C2)nc1 ; Cc1cccc(C(F)(F)F)c1COc1cnc(N2CCC[C@@H](O)C2)nc1 ; O=C1OC(CO)CN1c1ncc(OCc2c(Cl)cccc2Cl)cn1]

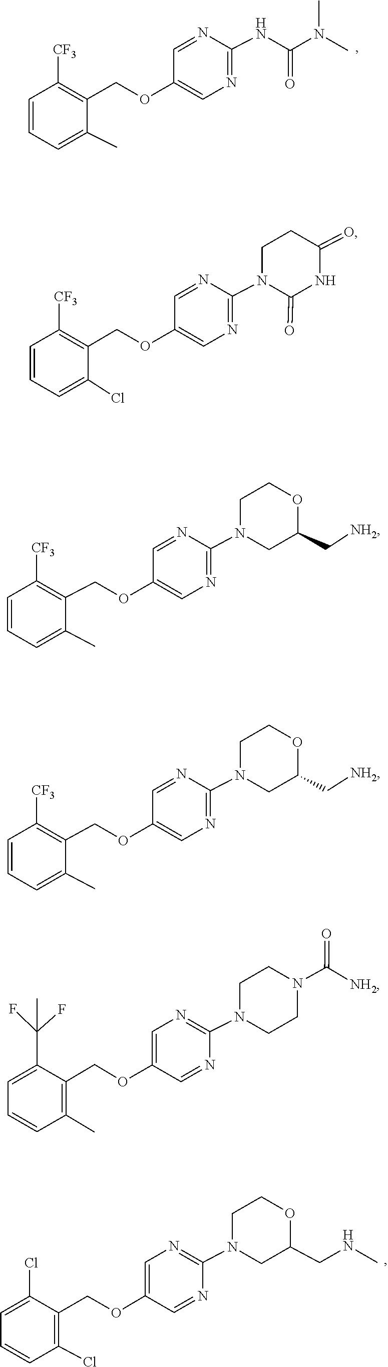 [SMILES: Cc1cccc(C(F)(F)F)c1COc1cnc(NC(=O)N(C)C)nc1 ; O=C1CCN(c2ncc(OCc3c(Cl)cccc3C(F)(F)F)cn2)C(=O)N1 ; Cc1cccc(C(F)(F)F)c1COc1cnc(N2CCO[C@@H](CN)C2)nc1 ; Cc1cccc(C(F)(F)F)c1COc1cnc(N2CCO[C@H](CN)C2)nc1 ; Cc1cccc(C(C)(F)F)c1COc1cnc(N2CCN(C(N)=O)CC2)nc1 ; CNCC1CN(c2ncc(OCc3c(Cl)cccc3Cl)cn2)CCO1]

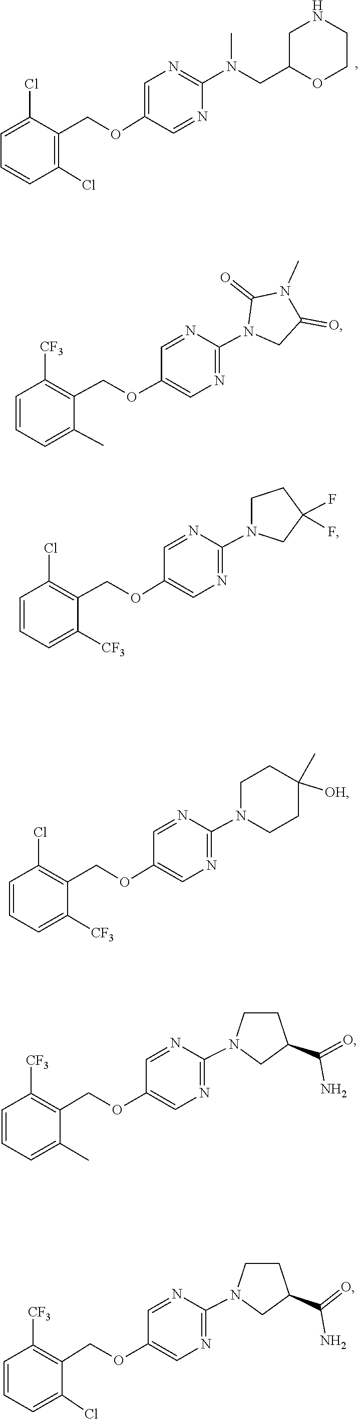 [SMILES: CN(CC1CNCCO1)c1ncc(OCc2c(Cl)cccc2Cl)cn1 ; Cc1cccc(C(F)(F)F)c1COc1cnc(N2CC(=O)N(C)C2=O)nc1 ; FC1(F)CCN(c2ncc(OCc3c(Cl)cccc3C(F)(F)F)cn2)C1 ; CC1(O)CCN(c2ncc(OCc3c(Cl)cccc3C(F)(F)F)cn2)CC1 ; Cc1cccc(C(F)(F)F)c1COc1cnc(N2CC[C@@H](C(N)=O)C2)nc1 ; NC(=O)[C@@H]1CCN(c2ncc(OCc3c(Cl)cccc3C(F)(F)F)cn2)C1]

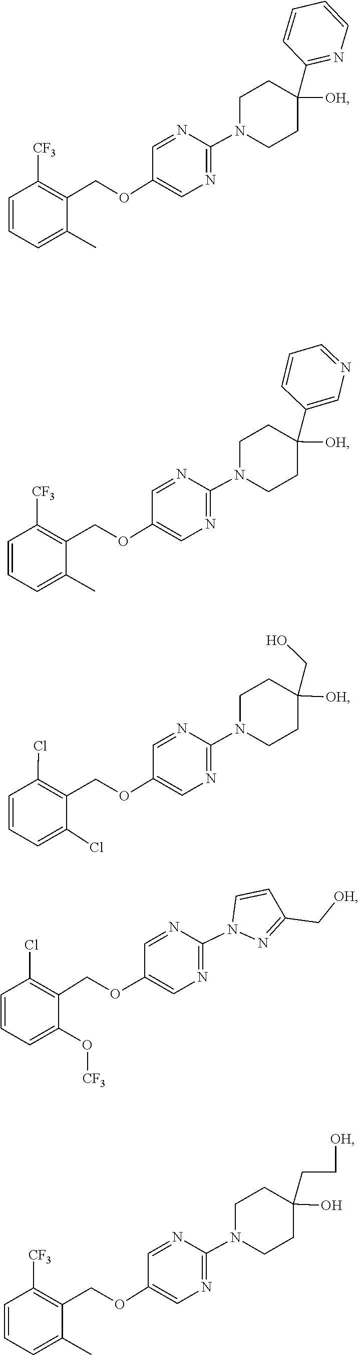 [SMILES: Cc1cccc(C(F)(F)F)c1COc1cnc(N2CCC(O)(c3ccccn3)CC2)nc1 ; Cc1cccc(C(F)(F)F)c1COc1cnc(N2CCC(O)(c3cccnc3)CC2)nc1 ; OCC1(O)CCN(c2ncc(OCc3c(Cl)cccc3Cl)cn2)CC1 ; OCc1ccn(-c2ncc(OCc3c(Cl)cccc3OC(F)(F)F)cn2)n1 ; Cc1cccc(C(F)(F)F)c1COc1cnc(N2CCC(O)(CCO)CC2)nc1]

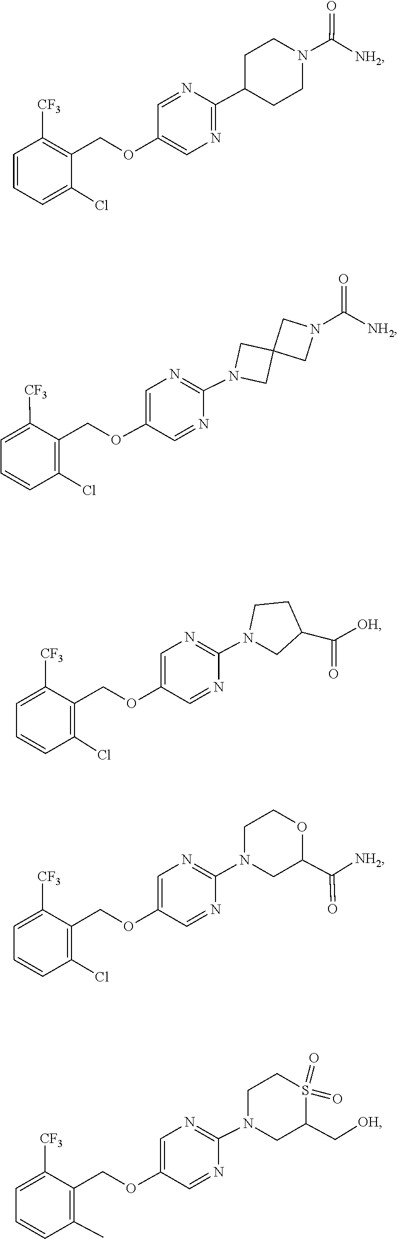 [SMILES: NC(=O)N1CCC(c2ncc(OCc3c(Cl)cccc3C(F)(F)F)cn2)CC1 ; NC(=O)N1CC2(C1)CN(c1ncc(OCc3c(Cl)cccc3C(F)(F)F)cn1)C2 ; O=C(O)C1CCN(c2ncc(OCc3c(Cl)cccc3C(F)(F)F)cn2)C1 ; NC(=O)C1CN(c2ncc(OCc3c(Cl)cccc3C(F)(F)F)cn2)CCO1 ; Cc1cccc(C(F)(F)F)c1COc1cnc(N2CCS(=O)(=O)C(CO)C2)nc1]

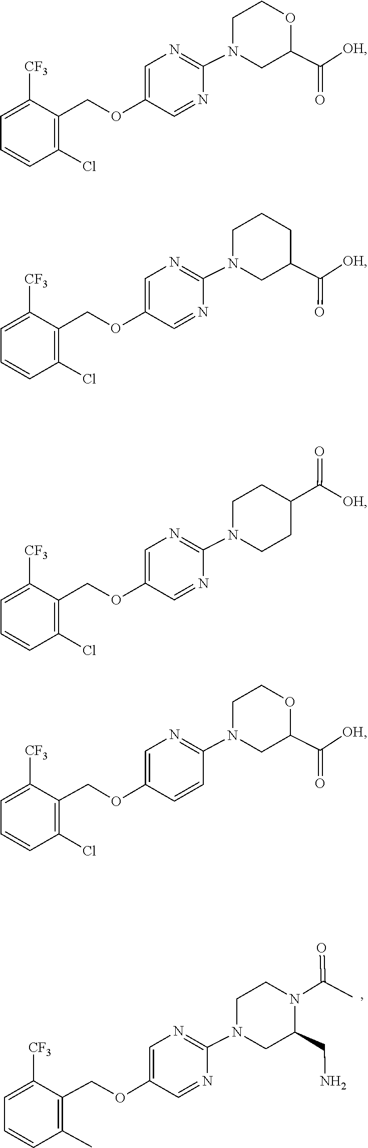 [SMILES: O=C(O)C1CN(c2ncc(OCc3c(Cl)cccc3C(F)(F)F)cn2)CCO1 ; O=C(O)C1CCCN(c2ncc(OCc3c(Cl)cccc3C(F)(F)F)cn2)C1 ; O=C(O)C1CCN(c2ncc(OCc3c(Cl)cccc3C(F)(F)F)cn2)CC1 ; O=C(O)C1CN(c2ccc(OCc3c(Cl)cccc3C(F)(F)F)cn2)CCO1 ; CC(=O)N1CCN(c2ncc(OCc3c(C)cccc3C(F)(F)F)cn2)C[C@@H]1CN]

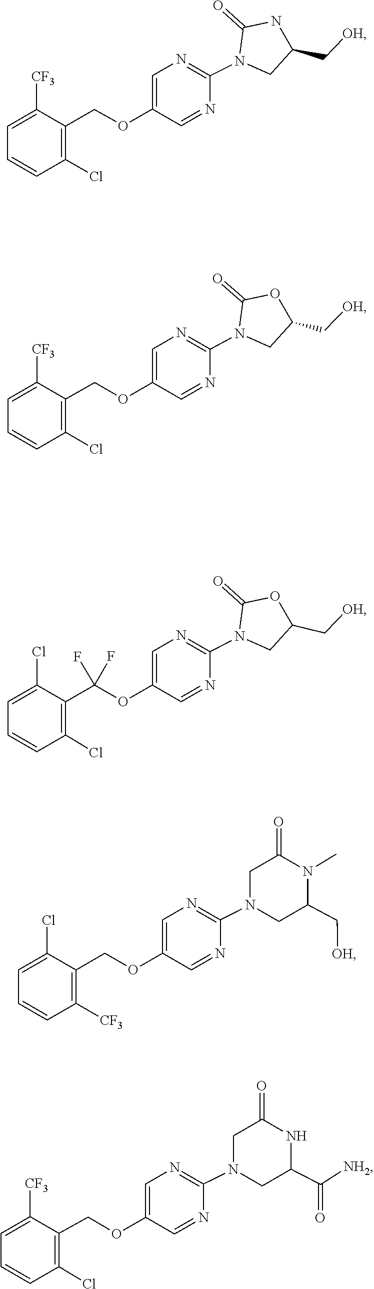

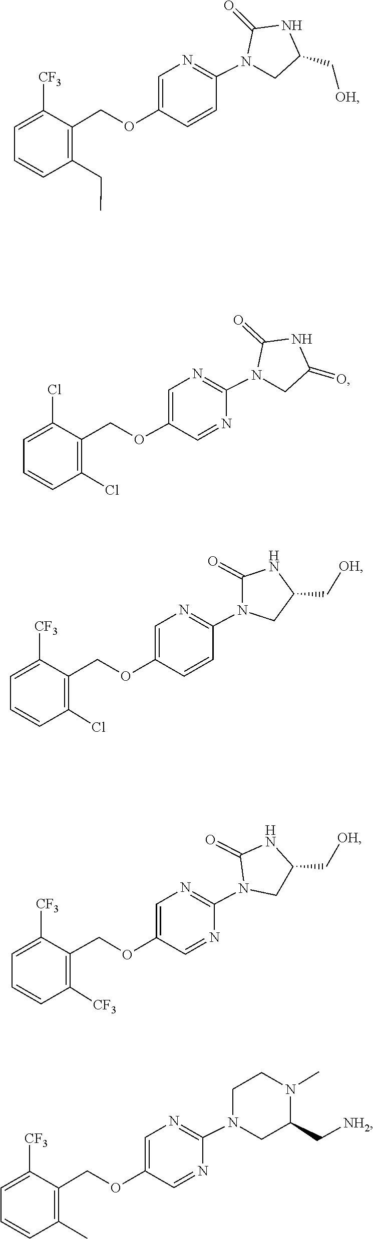 [SMILES: CCc1cccc(C(F)(F)F)c1COc1ccc(N2C[C@@H](CO)NC2=O)nc1 ; O=C1CN(c2ncc(OCc3c(Cl)cccc3Cl)cn2)C(=O)N1 ; O=C1N[C@H](CO)CN1c1ccc(OCc2c(Cl)cccc2C(F)(F)F)cn1 ; O=C1N[C@H](CO)CN1c1ncc(OCc2c(C(F)(F)F)cccc2C(F)(F)F)cn1 ; Cc1cccc(C(F)(F)F)c1COc1cnc(N2CCN(C)[C@@H](CN)C2)nc1]

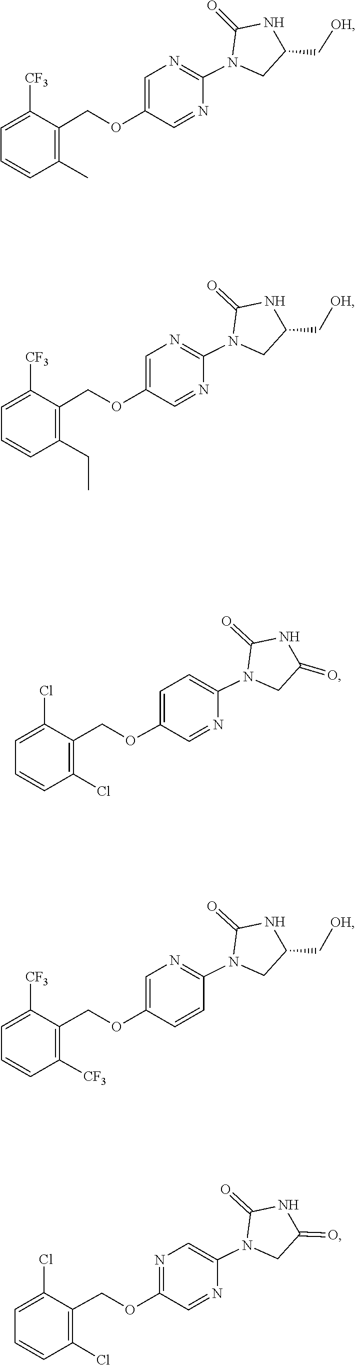 [SMILES: Cc1cccc(C(F)(F)F)c1COc1cnc(N2C[C@@H](CO)NC2=O)nc1 ; CCc1cccc(C(F)(F)F)c1COc1cnc(N2C[C@@H](CO)NC2=O)nc1 ; O=C1CN(c2ccc(OCc3c(Cl)cccc3Cl)cn2)C(=O)N1 ; O=C1N[C@H](CO)CN1c1ccc(OCc2c(C(F)(F)F)cccc2C(F)(F)F)cn1 ; O=C1CN(c2cnc(OCc3c(Cl)cccc3Cl)cn2)C(=O)N1]

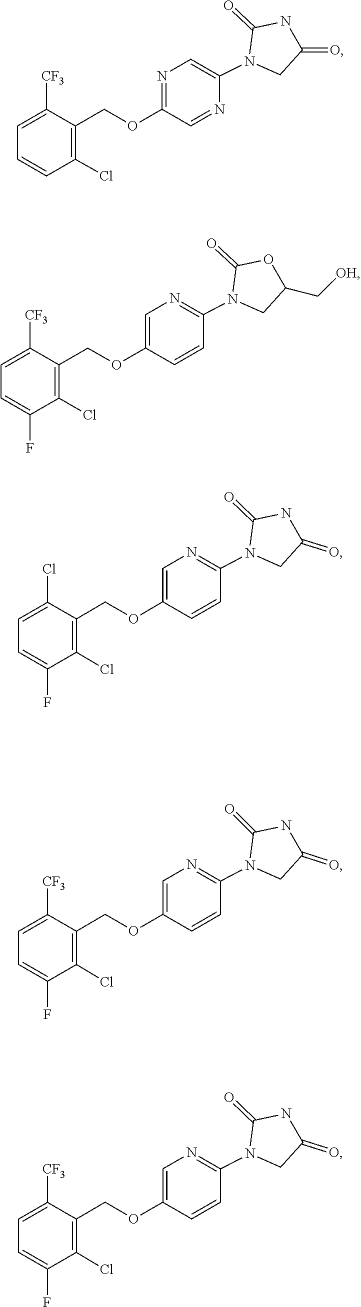

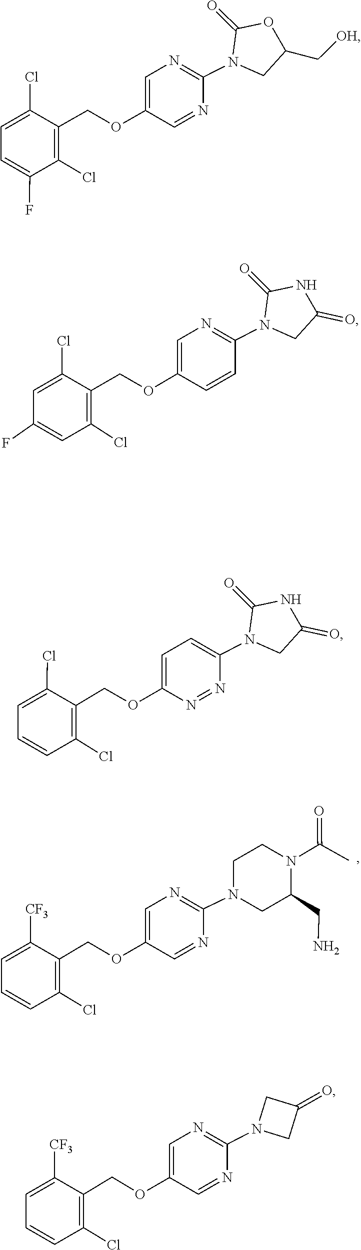 [SMILES: O=C1OC(CO)CN1c1ncc(OCc2c(Cl)ccc(F)c2Cl)cn1 ; O=C1CN(c2ccc(OCc3c(Cl)cc(F)cc3Cl)cn2)C(=O)N1 ; O=C1CN(c2ccc(OCc3c(Cl)cccc3Cl)nn2)C(=O)N1 ; CC(=O)N1CCN(c2ncc(OCc3c(Cl)cccc3C(F)(F)F)cn2)C[C@@H]1CN ; O=C1CN(c2ncc(OCc3c(Cl)cccc3C(F)(F)F)cn2)C1]

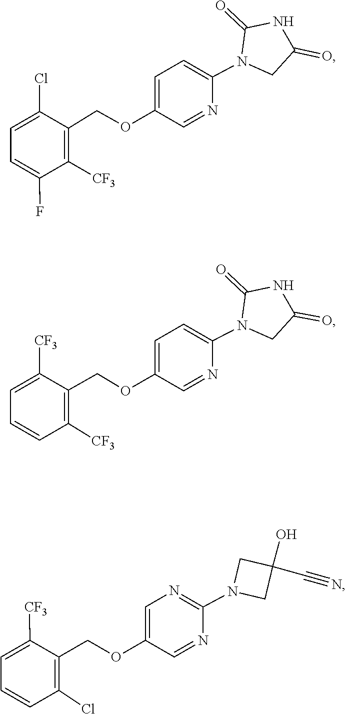 [SMILES: O=C1CN(c2ccc(OCc3c(Cl)ccc(F)c3C(F)(F)F)cn2)C(=O)N1 ; O=C1CN(c2ccc(OCc3c(C(F)(F)F)cccc3C(F)(F)F)cn2)C(=O)N1 ; N#CC1(O)CN(c2ncc(OCc3c(Cl)cccc3C(F)(F)F)cn2)C1]

and pharmaceutically acceptable salts thereof.

In some embodiments, the readthrough modulator compounds describe herein may be selected from the group consisting of:

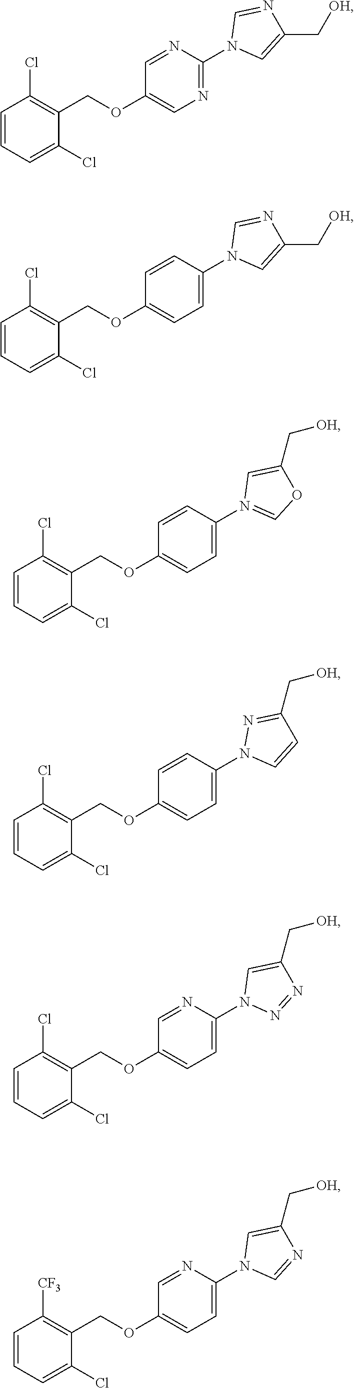

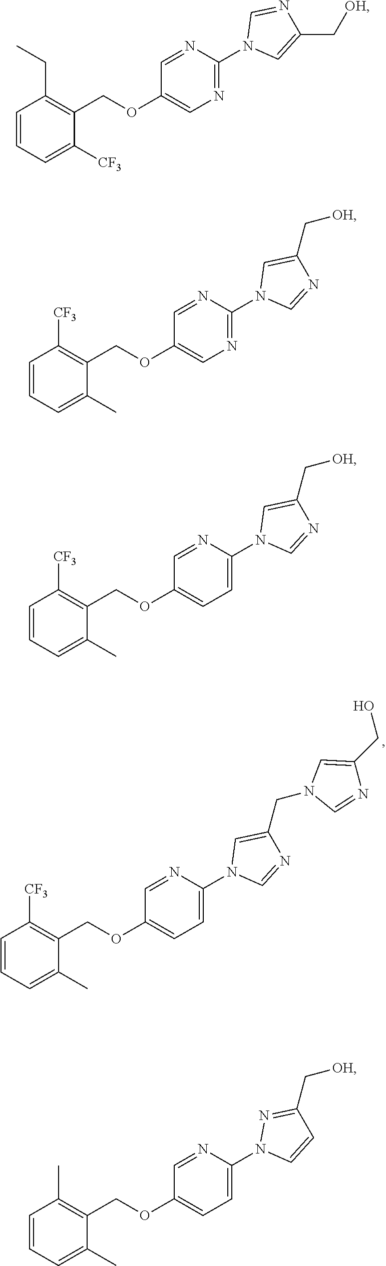 [SMILES: CCc1cccc(C(F)(F)F)c1COc1cnc(-n2cnc(CO)c2)nc1 ; Cc1cccc(C(F)(F)F)c1COc1cnc(-n2cnc(CO)c2)nc1 ; Cc1cccc(C(F)(F)F)c1COc1ccc(-n2cnc(CO)c2)nc1 ; Cc1cccc(C(F)(F)F)c1COc1ccc(-n2cnc(Cn3cnc(CO)c3)c2)nc1 ; Cc1cccc(C)c1COc1ccc(-n2ccc(CO)n2)nc1]

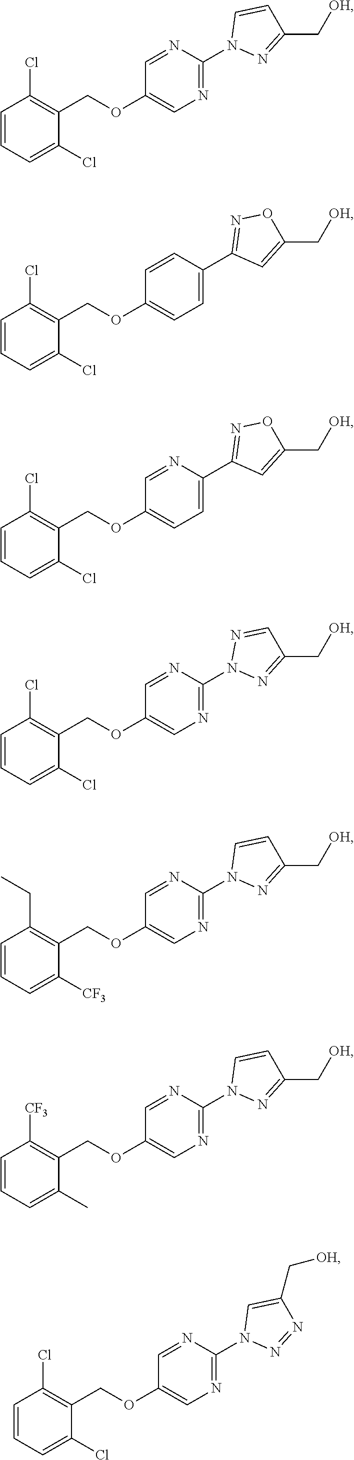

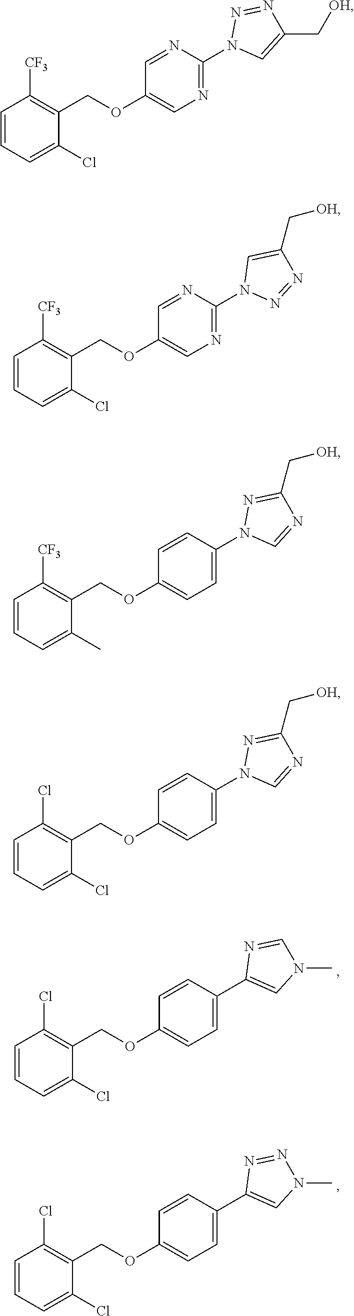 [SMILES: OCc1cn(-c2ncc(OCc3c(Cl)cccc3C(F)(F)F)cn2)nn1 ; OCc1cn(-c2ncc(OCc3c(Cl)cccc3C(F)(F)F)cn2)nn1 ; Cc1cccc(C(F)(F)F)c1COc1ccc(-n2cnc(CO)n2)cc1 ; OCc1ncn(-c2ccc(OCc3c(Cl)cccc3Cl)cc2)n1 ; Cn1cnc(-c2ccc(OCc3c(Cl)cccc3Cl)cc2)c1 ; Cn1cc(-c2ccc(OCc3c(Cl)cccc3Cl)cc2)nn1]

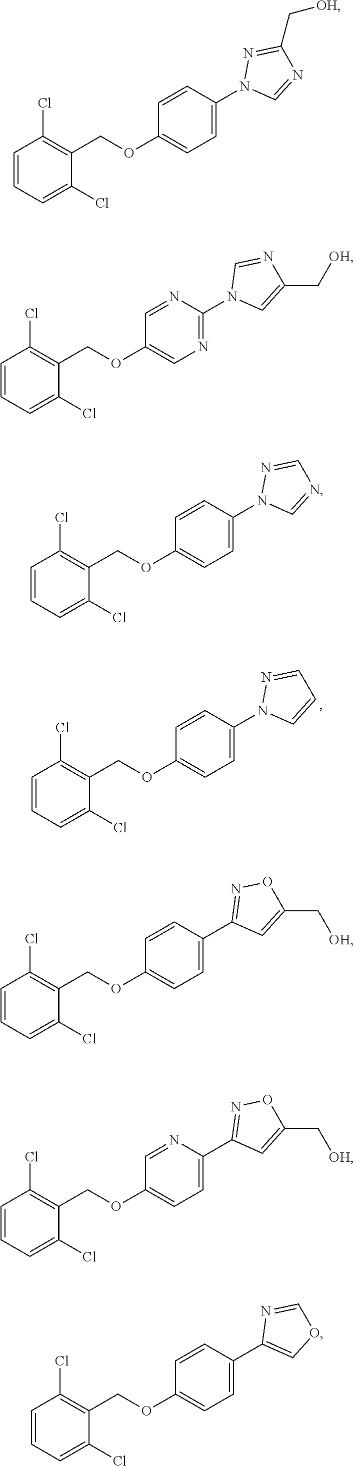

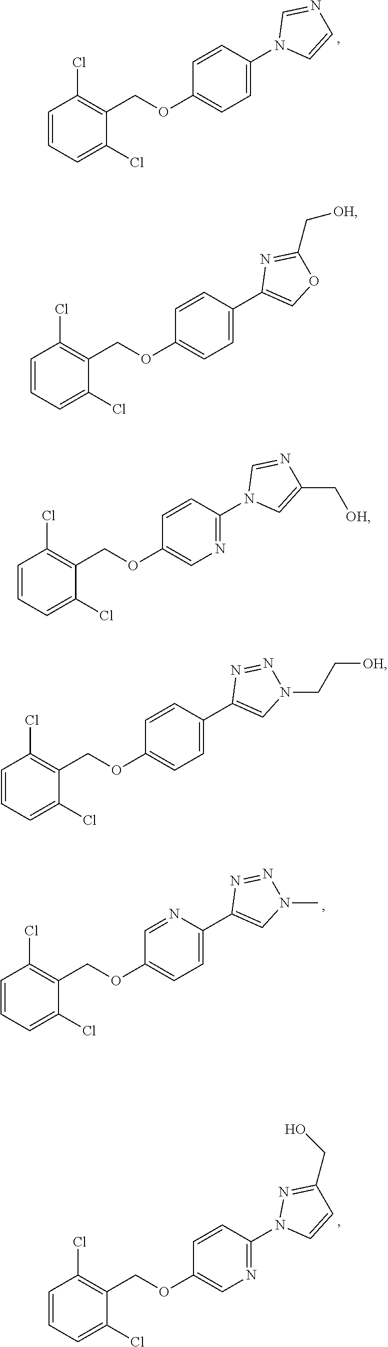 [SMILES: Clc1cccc(Cl)c1COc1ccc(-n2ccnc2)cc1 ; OCc1nc(-c2ccc(OCc3c(Cl)cccc3Cl)cc2)co1 ; OCc1cn(-c2ccc(OCc3c(Cl)cccc3Cl)cn2)cn1 ; OCCn1cc(-c2ccc(OCc3c(Cl)cccc3Cl)cc2)nn1 ; Cn1cc(-c2ccc(OCc3c(Cl)cccc3Cl)cn2)nn1 ; OCc1ccn(-c2ccc(OCc3c(Cl)cccc3Cl)cn2)n1]

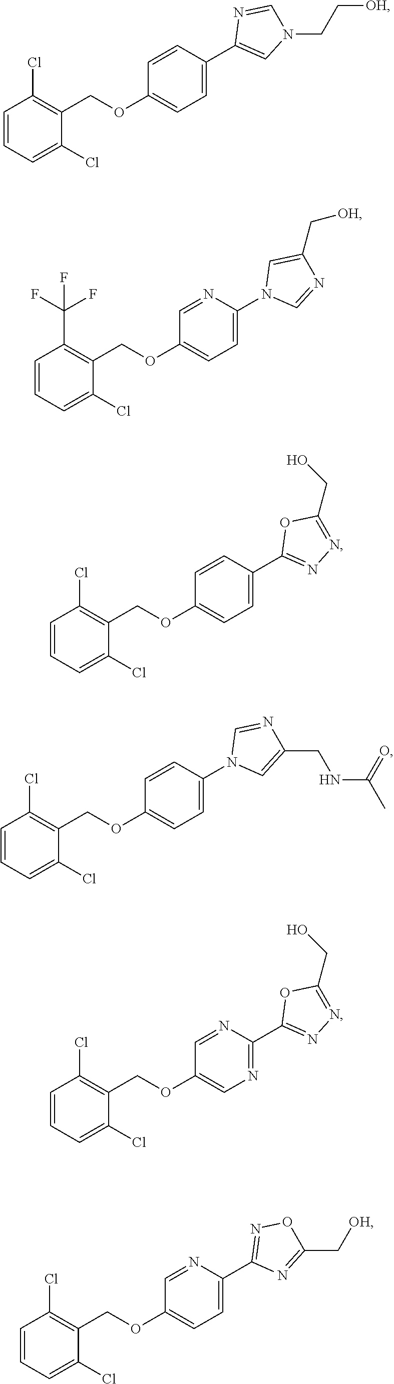 [SMILES: OCCn1cnc(-c2ccc(OCc3c(Cl)cccc3Cl)cc2)c1 ; OCc1cn(-c2ccc(OCc3c(Cl)cccc3C(F)(F)F)cn2)cn1 ; OCc1nnc(-c2ccc(OCc3c(Cl)cccc3Cl)cc2)o1 ; CC(=O)NCc1cn(-c2ccc(OCc3c(Cl)cccc3Cl)cc2)cn1 ; OCc1nnc(-c2ncc(OCc3c(Cl)cccc3Cl)cn2)o1 ; OCc1nc(-c2ccc(OCc3c(Cl)cccc3Cl)cn2)no1]

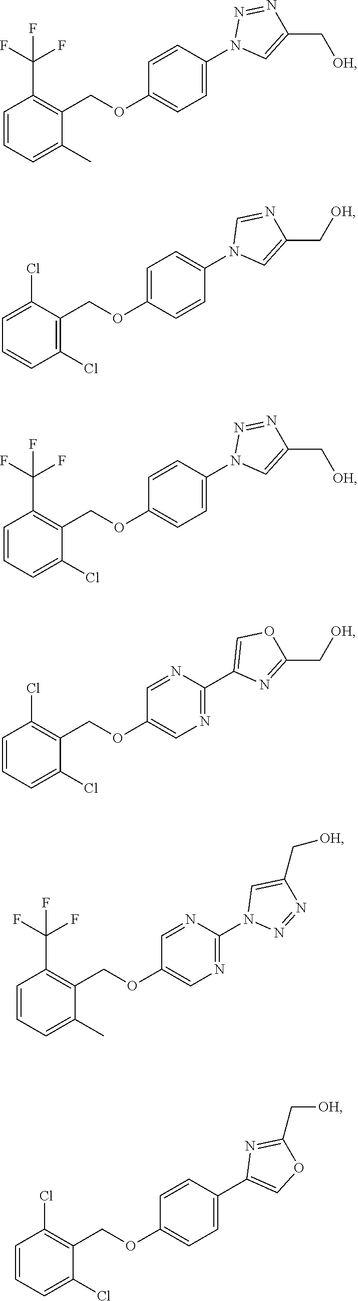 [SMILES: Cc1cccc(C(F)(F)F)c1COc1ccc(-n2cc(CO)nn2)cc1 ; OCc1cn(-c2ccc(OCc3c(Cl)cccc3Cl)cc2)cn1 ; OCc1cn(-c2ccc(OCc3c(Cl)cccc3C(F)(F)F)cc2)nn1 ; OCc1nc(-c2ncc(OCc3c(Cl)cccc3Cl)cn2)co1 ; Cc1cccc(C(F)(F)F)c1COc1cnc(-n2cc(CO)nn2)nc1 ; OCc1nc(-c2ccc(OCc3c(Cl)cccc3Cl)cc2)co1]

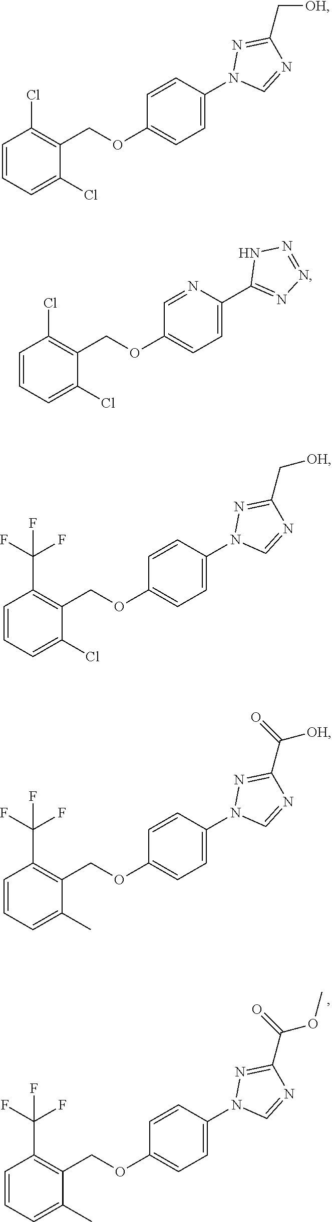 [SMILES: OCc1ncn(-c2ccc(OCc3c(Cl)cccc3Cl)cc2)n1 ; Clc1cccc(Cl)c1COc1ccc(-c2nnn[nH]2)nc1 ; OCc1ncn(-c2ccc(OCc3c(Cl)cccc3C(F)(F)F)cc2)n1 ; Cc1cccc(C(F)(F)F)c1COc1ccc(-n2cnc(C(=O)O)n2)cc1 ; COC(=O)c1ncn(-c2ccc(OCc3c(C)cccc3C(F)(F)F)cc2)n1]

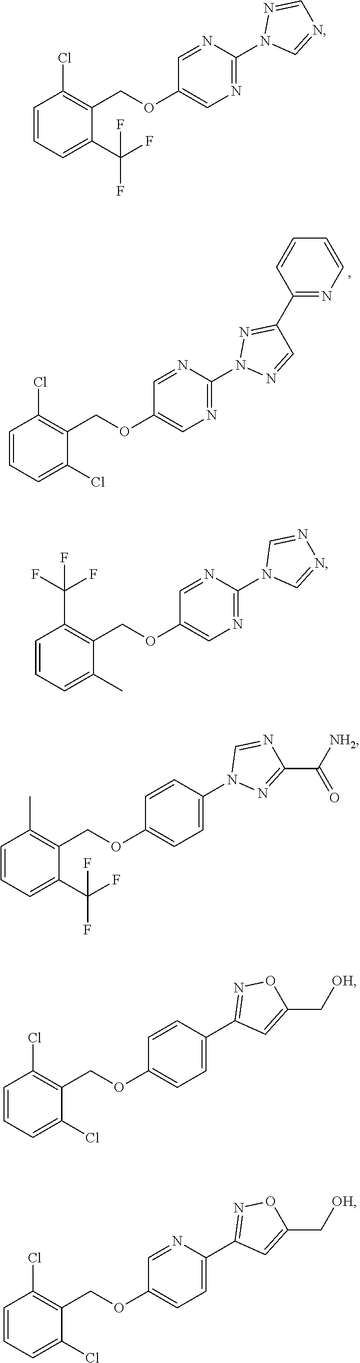 [SMILES: FC(F)(F)c1cccc(Cl)c1COc1cnc(-n2cncn2)nc1 ; Clc1cccc(Cl)c1COc1cnc(-n2ncc(-c3ccccn3)n2)nc1 ; Cc1cccc(C(F)(F)F)c1COc1cnc(-n2cnnc2)nc1 ; Cc1cccc(C(F)(F)F)c1COc1ccc(-n2cnc(C(N)=O)n2)cc1 ; OCc1cc(-c2ccc(OCc3c(Cl)cccc3Cl)cc2)no1 ; OCc1cc(-c2ccc(OCc3c(Cl)cccc3Cl)cn2)no1]

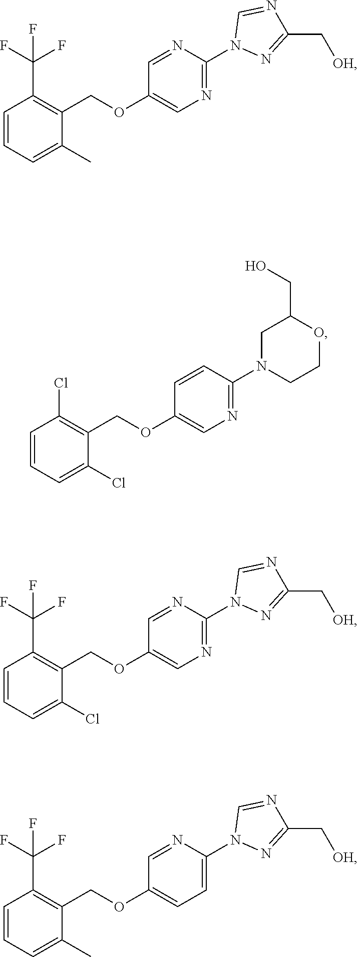 [SMILES: Cc1cccc(C(F)(F)F)c1COc1cnc(-n2cnc(CO)n2)nc1 ; OCC1CN(c2ccc(OCc3c(Cl)cccc3Cl)cn2)CCO1 ; OCc1ncn(-c2ncc(OCc3c(Cl)cccc3C(F)(F)F)cn2)n1 ; Cc1cccc(C(F)(F)F)c1COc1ccc(-n2cnc(CO)n2)nc1]

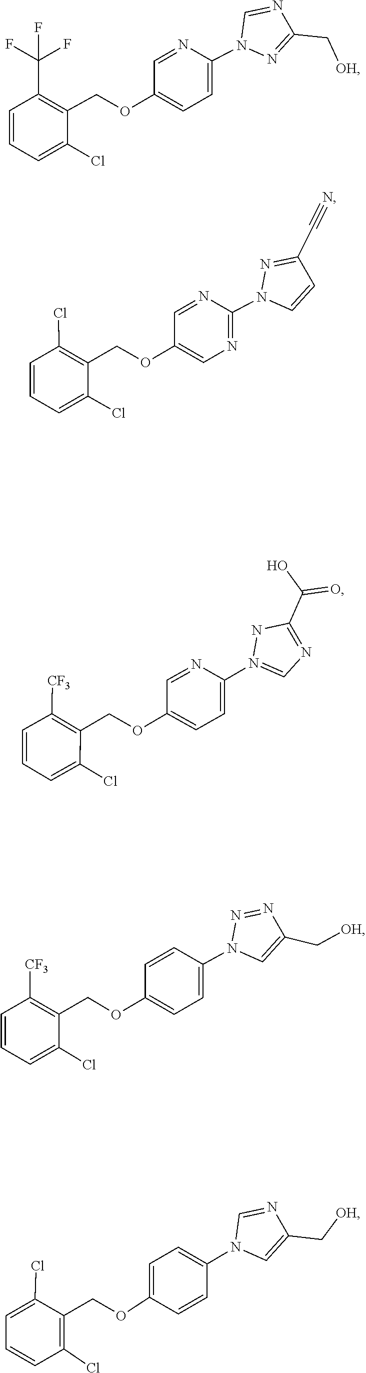

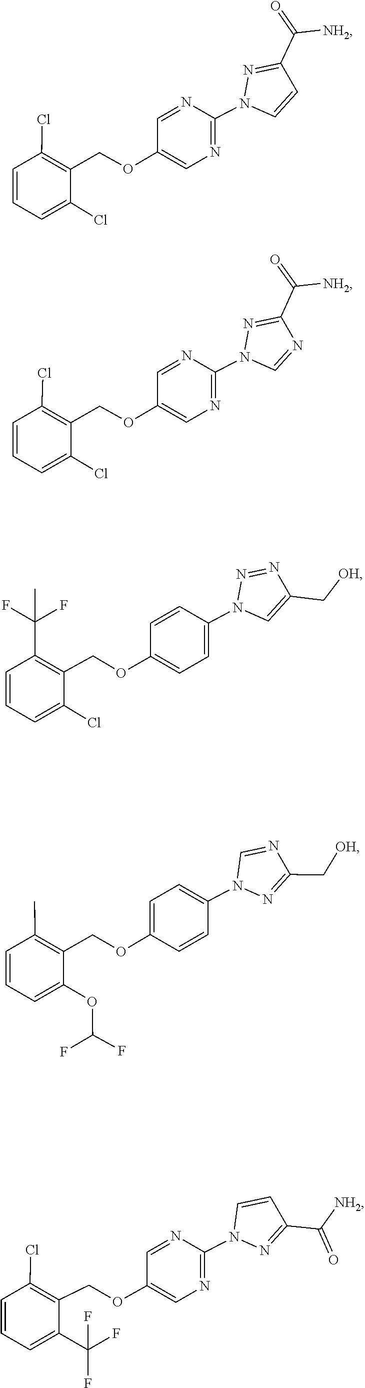 [SMILES: NC(=O)c1ccn(-c2ncc(OCc3c(Cl)cccc3Cl)cn2)n1 ; NC(=O)c1ncn(-c2ncc(OCc3c(Cl)cccc3Cl)cn2)n1 ; CC(F)(F)c1cccc(Cl)c1COc1ccc(-n2cc(CO)nn2)cc1 ; Cc1cccc(OC(F)F)c1COc1ccc(-n2cnc(CO)n2)cc1 ; NC(=O)c1ccn(-c2ncc(OCc3c(Cl)cccc3C(F)(F)F)cn2)n1]

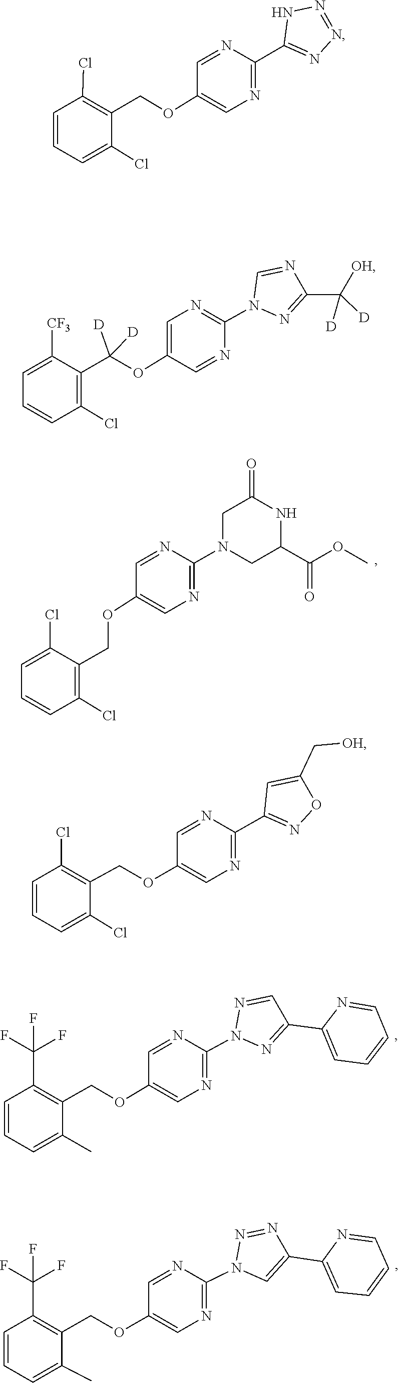 [SMILES: Clc1cccc(Cl)c1COc1cnc(-c2nnn[nH]2)nc1 ; [2H]C([2H])(O)c1ncn(-c2ncc(OC([2H])([2H])c3c(Cl)cccc3C(F)(F)F)cn2)n1 ; COC(=O)C1CN(c2ncc(OCc3c(Cl)cccc3Cl)cn2)CC(=O)N1 ; OCc1cc(-c2ncc(OCc3c(Cl)cccc3Cl)cn2)no1 ; Cc1cccc(C(F)(F)F)c1COc1cnc(-n2ncc(-c3ccccn3)n2)nc1 ; Cc1cccc(C(F)(F)F)c1COc1cnc(-n2cc(-c3ccccn3)nn2)nc1]

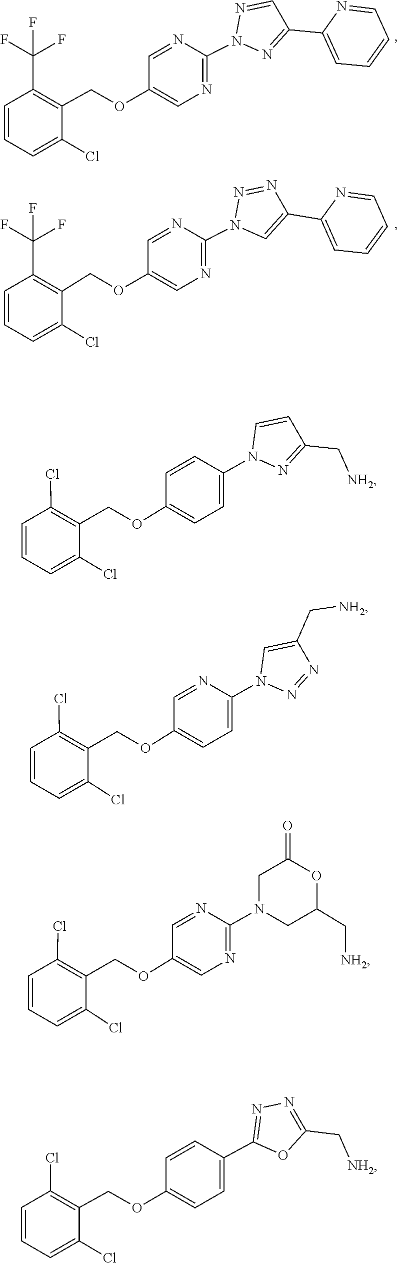 [SMILES: FC(F)(F)c1cccc(Cl)c1COc1cnc(-n2ncc(-c3ccccn3)n2)nc1 ; FC(F)(F)c1cccc(Cl)c1COc1cnc(-n2cc(-c3ccccn3)nn2)nc1 ; NCc1ccn(-c2ccc(OCc3c(Cl)cccc3Cl)cc2)n1 ; NCc1cn(-c2ccc(OCc3c(Cl)cccc3Cl)cn2)nn1 ; NCC1CN(c2ncc(OCc3c(Cl)cccc3Cl)cn2)CC(=O)O1 ; NCc1nnc(-c2ccc(OCc3c(Cl)cccc3Cl)cc2)o1]

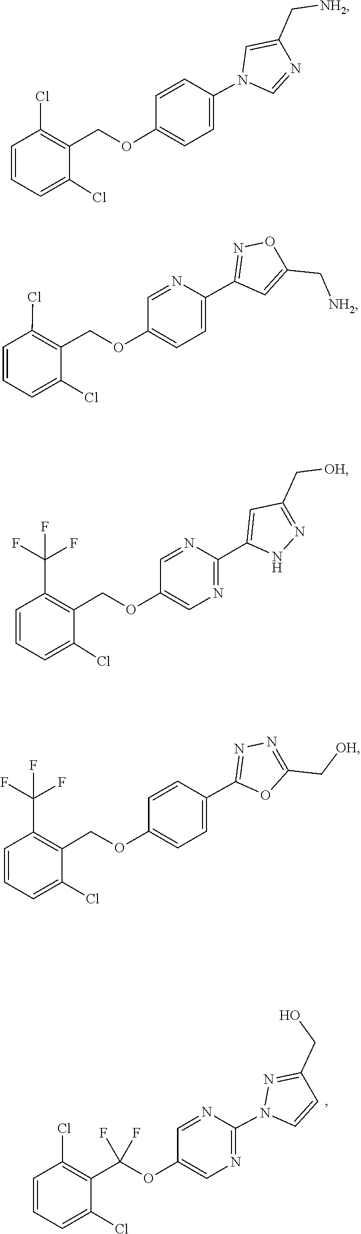 [SMILES: NCc1cn(-c2ccc(OCc3c(Cl)cccc3Cl)cc2)cn1 ; NCc1cc(-c2ccc(OCc3c(Cl)cccc3Cl)cn2)no1 ; OCc1cc(-c2ncc(OCc3c(Cl)cccc3C(F)(F)F)cn2)[nH]n1 ; OCc1nnc(-c2ccc(OCc3c(Cl)cccc3C(F)(F)F)cc2)o1 ; OCc1ccn(-c2ncc(OC(F)(F)c3c(Cl)cccc3Cl)cn2)n1]

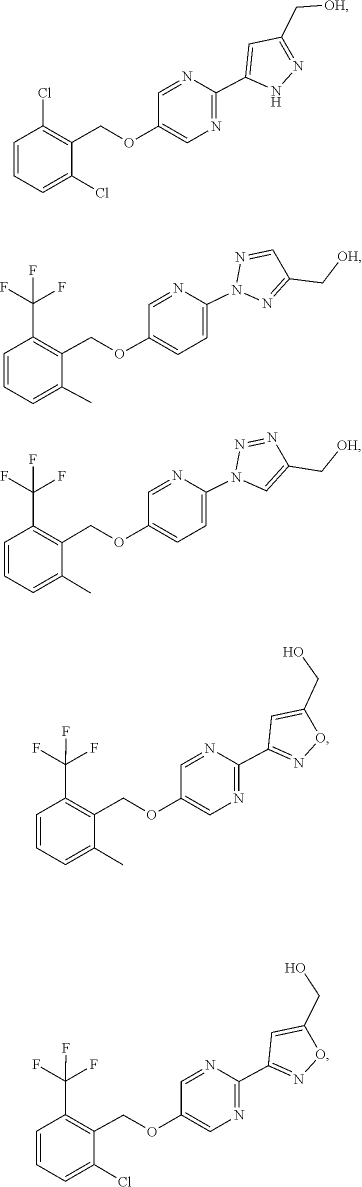 [SMILES: OCc1cc(-c2ncc(OCc3c(Cl)cccc3Cl)cn2)[nH]n1 ; Cc1cccc(C(F)(F)F)c1COc1ccc(-n2ncc(CO)n2)nc1 ; Cc1cccc(C(F)(F)F)c1COc1ccc(-n2cc(CO)nn2)nc1 ; Cc1cccc(C(F)(F)F)c1COc1cnc(-c2cc(CO)on2)nc1 ; OCc1cc(-c2ncc(OCc3c(Cl)cccc3C(F)(F)F)cn2)no1]

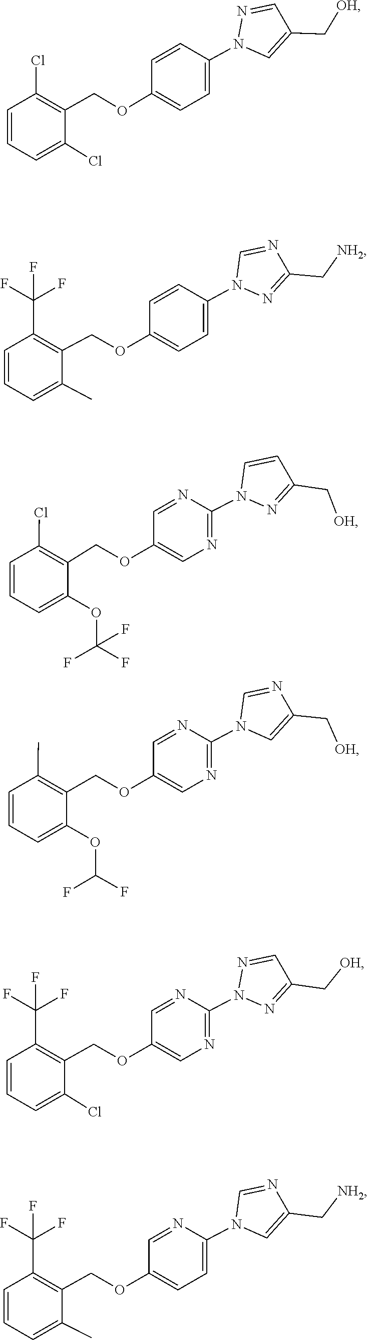 [SMILES: OCc1cnn(-c2ccc(OCc3c(Cl)cccc3Cl)cc2)c1 ; Cc1cccc(C(F)(F)F)c1COc1ccc(-n2cnc(CN)n2)cc1 ; OCc1ccn(-c2ncc(OCc3c(Cl)cccc3OC(F)(F)F)cn2)n1 ; Cc1cccc(OC(F)F)c1COc1cnc(-n2cnc(CO)c2)nc1 ; OCc1cnn(-c2ncc(OCc3c(Cl)cccc3C(F)(F)F)cn2)n1 ; Cc1cccc(C(F)(F)F)c1COc1ccc(-n2cnc(CN)c2)nc1]

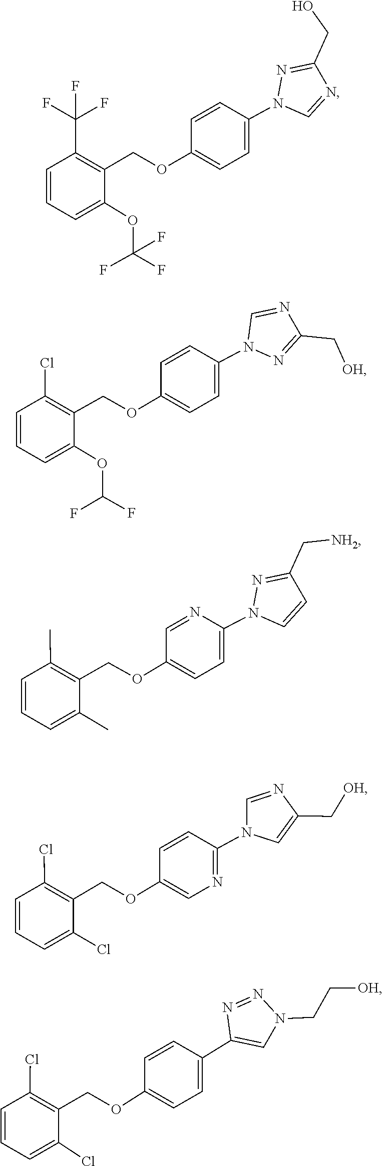 [SMILES: OCc1ncn(-c2ccc(OCc3c(OC(F)(F)F)cccc3C(F)(F)F)cc2)n1 ; OCc1ncn(-c2ccc(OCc3c(Cl)cccc3OC(F)F)cc2)n1 ; Cc1cccc(C)c1COc1ccc(-n2ccc(CN)n2)nc1 ; OCc1cn(-c2ccc(OCc3c(Cl)cccc3Cl)cn2)cn1 ; OCCn1cc(-c2ccc(OCc3c(Cl)cccc3Cl)cc2)nn1]

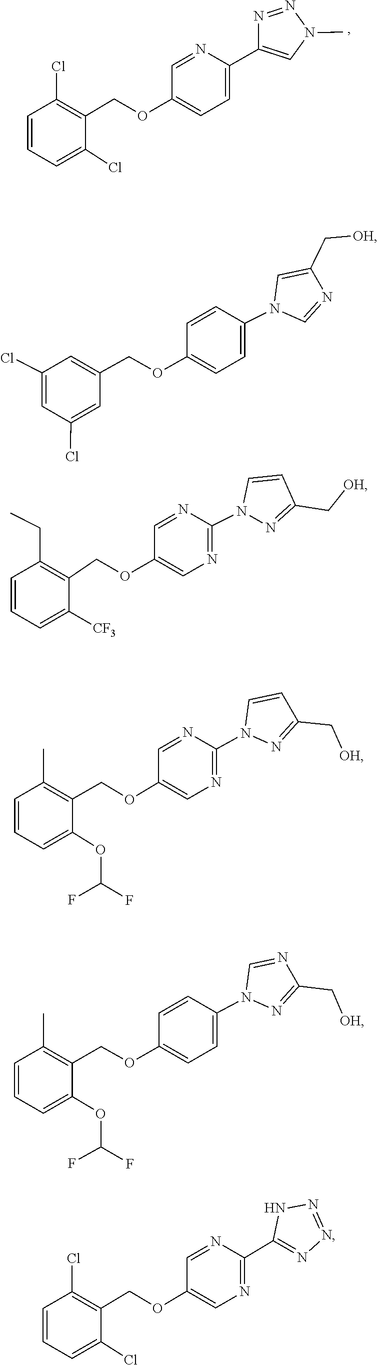

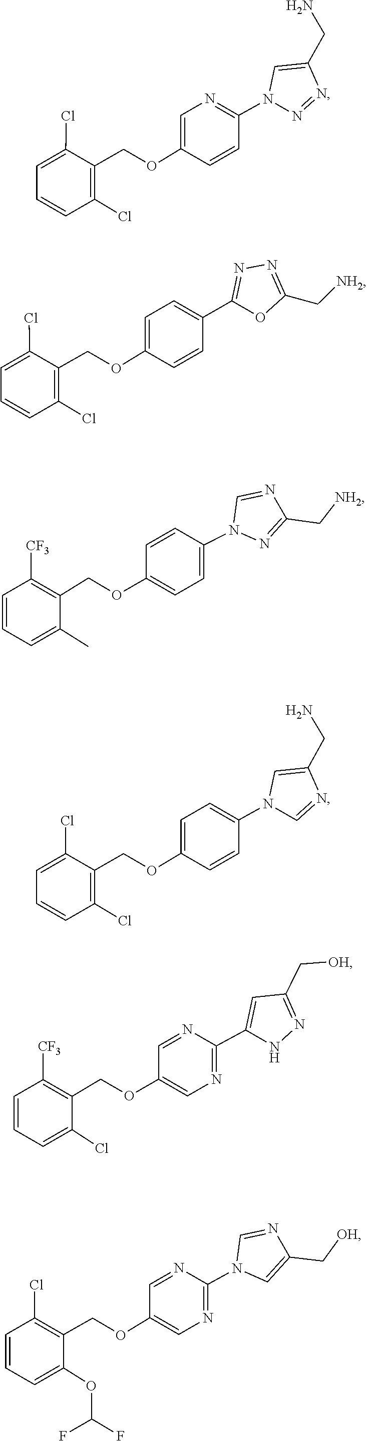 [SMILES: NCc1cn(-c2ccc(OCc3c(Cl)cccc3Cl)cn2)nn1 ; NCc1nnc(-c2ccc(OCc3c(Cl)cccc3Cl)cc2)o1 ; Cc1cccc(C(F)(F)F)c1COc1ccc(-n2cnc(CN)n2)cc1 ; NCc1cn(-c2ccc(OCc3c(Cl)cccc3Cl)cc2)cn1 ; OCc1cc(-c2ncc(OCc3c(Cl)cccc3C(F)(F)F)cn2)[nH]n1 ; OCc1cn(-c2ncc(OCc3c(Cl)cccc3OC(F)F)cn2)cn1]

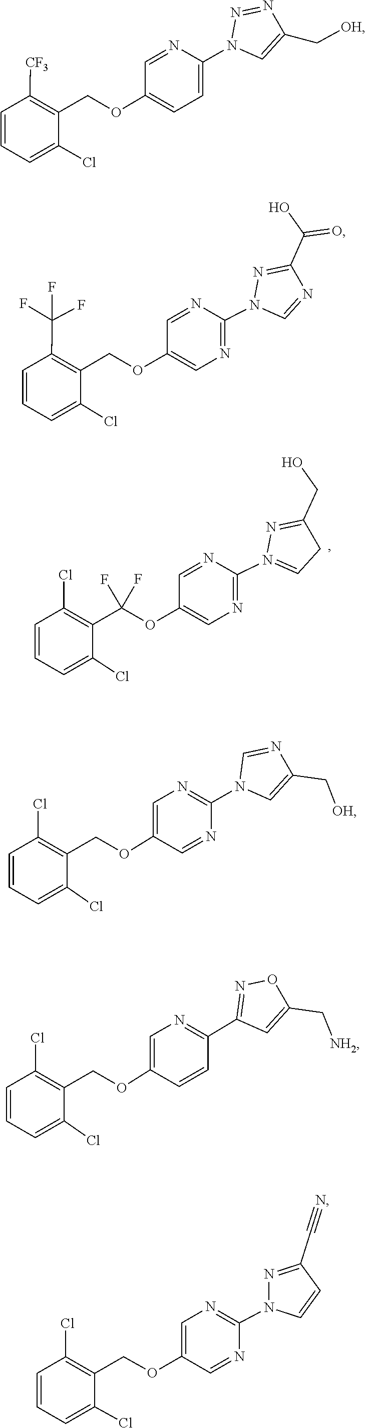

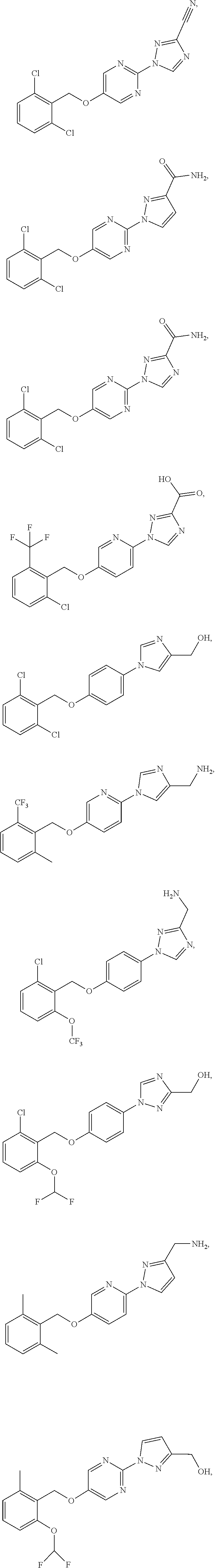

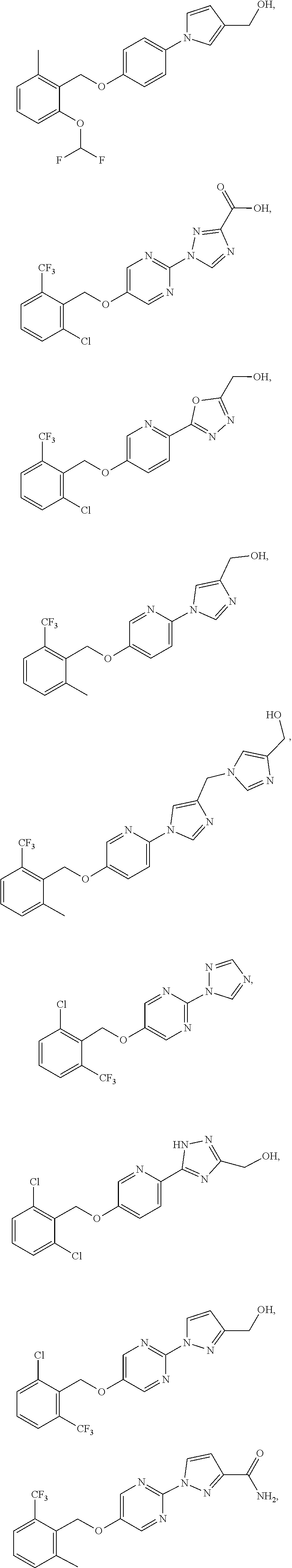

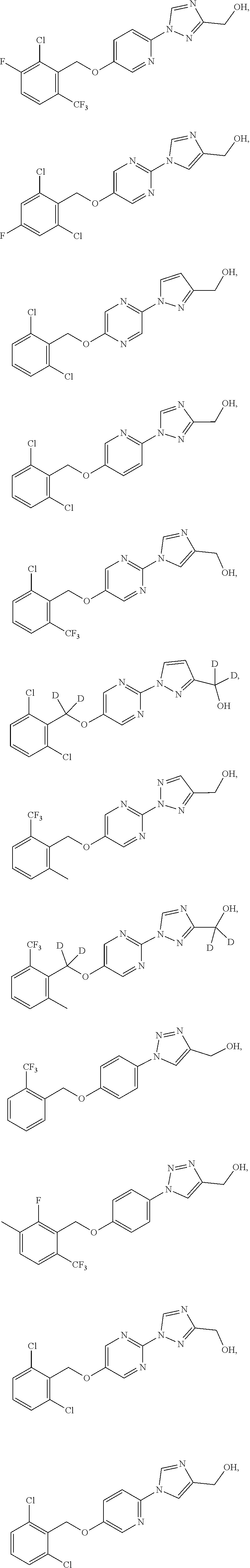

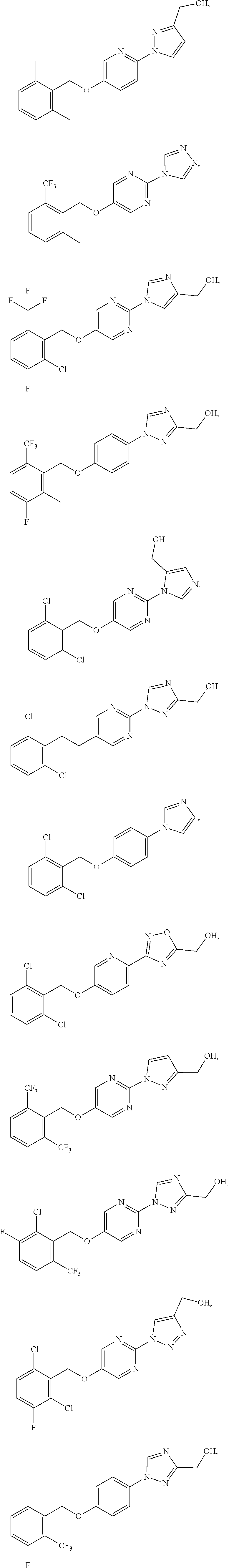

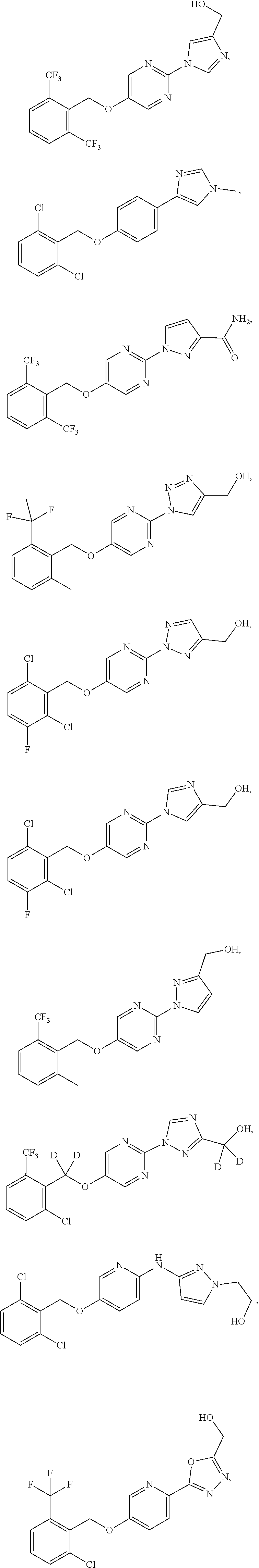

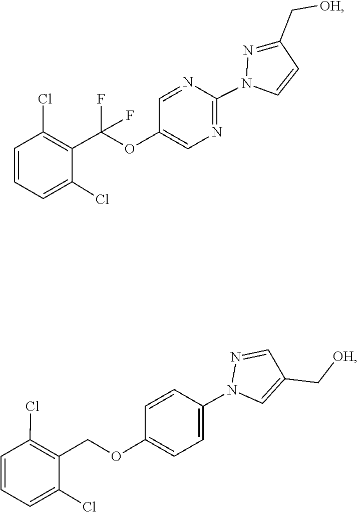 [SMILES: OCc1ccn(-c2ncc(OC(F)(F)c3c(Cl)cccc3Cl)cn2)n1 ; OCc1cnn(-c2ccc(OCc3c(Cl)cccc3Cl)cc2)c1]

and pharmaceutically acceptable salts thereof.

In some embodiments, the readthrough modulator compounds describe herein may be selected from the group consisting of:

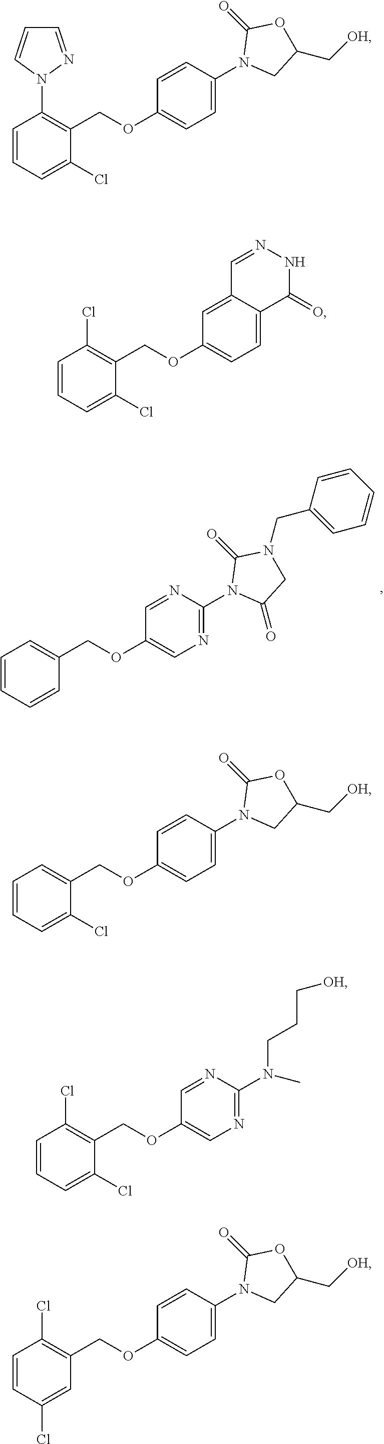 [SMILES: O=C1OC(CO)CN1c1ccc(OCc2c(Cl)cccc2-n2cccn2)cc1 ; O=c1[nH]ncc2cc(OCc3c(Cl)cccc3Cl)ccc12 ; O=C1CN(Cc2ccccc2)C(=O)N1c1ncc(OCc2ccccc2)cn1 ; O=C1OC(CO)CN1c1ccc(OCc2ccccc2Cl)cc1 ; CN(CCCO)c1ncc(OCc2c(Cl)cccc2Cl)cn1 ; O=C1OC(CO)CN1c1ccc(OCc2cc(Cl)ccc2Cl)cc1]

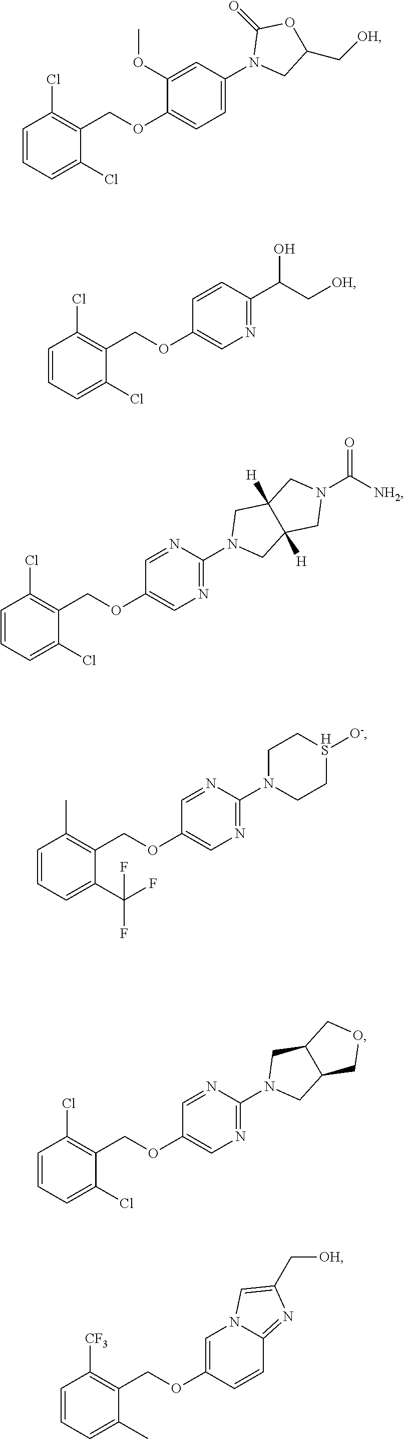

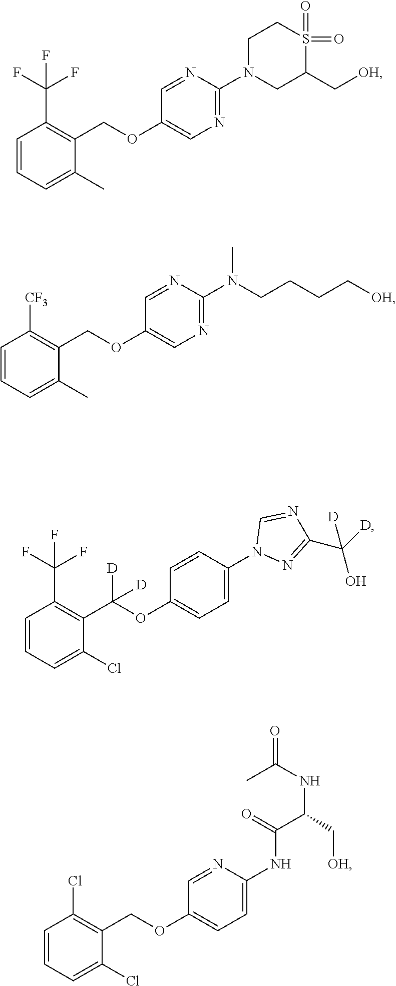

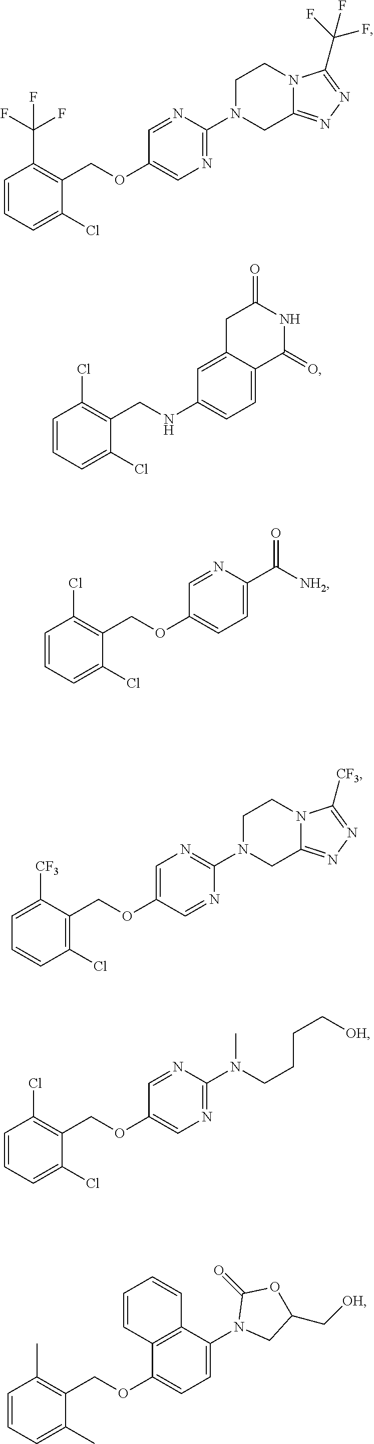 [SMILES: FC(F)(F)c1cccc(Cl)c1COc1cnc(N2CCn3c(nnc3C(F)(F)F)C2)nc1 ; O=C1Cc2cc(NCc3c(Cl)cccc3Cl)ccc2C(=O)N1 ; NC(=O)c1ccc(OCc2c(Cl)cccc2Cl)cn1 ; FC(F)(F)c1cccc(Cl)c1COc1cnc(N2CCn3c(nnc3C(F)(F)F)C2)nc1 ; CN(CCCCO)c1ncc(OCc2c(Cl)cccc2Cl)cn1 ; Cc1cccc(C)c1COc1ccc(N2CC(CO)OC2=O)c2ccccc12]

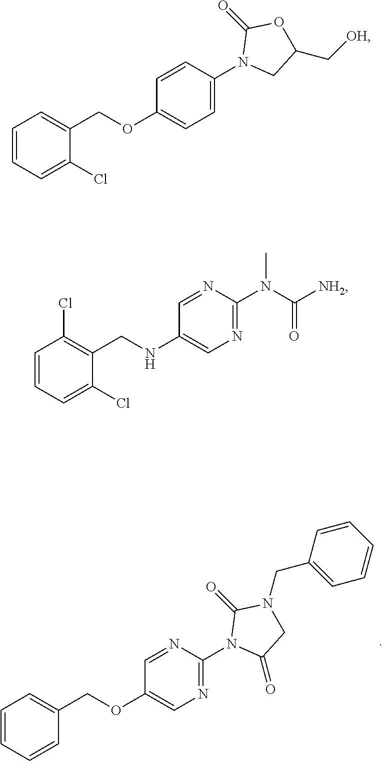 [SMILES: O=C1OC(CO)CN1c1ccc(OCc2ccccc2Cl)cc1 ; CN(C(N)=O)c1ncc(NCc2c(Cl)cccc2Cl)cn1 ; O=C1CN(Cc2ccccc2)C(=O)N1c1ncc(OCc2ccccc2)cn1]

and pharmaceutically acceptable salts thereof.

### Definitions

“Subject” as used herein, means a human or a non-human mammal including but not limited to a dog, cat, horse, donkey, mule, cow, domestic buffalo, camel, llama, alpaca, bison, yak, goat, sheep, pig, elk, deer, domestic antelope, or a non-human primate selected for treatment or therapy.

“Subject in need thereof” means a subject identified as in need of a therapy or treatment.

A therapeutic effect relieves, to some extent, one or more of the symptoms of a disease or disorder, and includes curing the disease or disorder. “Curing” means that the symptoms of active disease are eliminated. However, certain long-term or permanent effects of the disease may exist even after a cure is obtained (such as extensive tissue damage).

The phrase “therapeutically effective amount” means an amount of a compound or a combination of compounds that ameliorates, attenuates or eliminates one or more of the symptoms of a particular disease or condition or prevents, modifies, or delays the onset of one or more of the symptoms of a particular disease or condition.

“Treat,” “treatment,” or “treating,” as used herein refers to administering a pharmaceutical composition for prophylactic and/or therapeutic purposes. The term “prophylactic treatment” refers to treating a patient who does not yet have the relevant disease or disorder, but who is susceptible to, or otherwise at risk of, a particular disease or disorder, whereby the treatment reduces the likelihood that the patient will develop the disease or disorder. The term “therapeutic treatment” refers to administering treatment to a patient already having a disease or disorder.

“Preventing” or “prevention” refers to delaying or forestalling the onset, development or progression of a condition or disease for a period of time, including weeks, months, or years.

“Amelioration” means a lessening of severity of at least one indicator of a condition or disease. In certain embodiments, amelioration includes a delay or slowing in the progression of one or more indicators of a condition or disease. The severity of indicators may be determined by subjective or objective measures which are known to those skilled in the art.

“Modulation” or “modulate” means a perturbation of function or activity. In certain embodiments, modulation means an increase in gene expression. In certain embodiments, modulation means a decrease in gene expression. In certain embodiments, modulation means an increase or decrease in total serum levels of a specific protein. In certain embodiments, modulation means an increase or decrease in free serum levels of a specific protein. In certain embodiments, modulation means an increase or decrease in total serum levels of a specific non-protein factor. In certain embodiments, modulation means an increase or decrease in free serum levels of a specific non-protein factor. In certain embodiments, modulation means an increase or decrease in total bioavailability of a specific protein. In certain embodiments, modulation means an increase or decrease in total bioavailability of a specific non-protein factor.

As used herein, a “readthrough modulator” refers to a compound that modulates readthrough of premature stop codons, where such modulation occurs upon treatment with the readthrough modulator alone and/or treatment with the readthrough modulator in combination with an aminoglycoside. In some embodiments, the readthrough modulator modulates readthrough of premature stop codons when administered in combination with a sub-effective, sub-optimal, or sub-maximal amount of an aminoglycoside.

“Administering” means providing a pharmaceutical agent or composition to a subject, and includes, but is not limited to, administering by a medical professional and self-administering.

Administration of the compounds disclosed herein or the pharmaceutically acceptable salts thereof, or other agents disclosed herein can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly. Oral and parenteral administrations are customary in treating the indications that are the subject of the preferred embodiments.

“Parenteral administration,” means administration through injection or infusion. Parenteral administration includes, but is not limited to, subcutaneous administration, intravenous administration, intramuscular administration, intraarterial administration, and intracranial administration.

“Subcutaneous administration” means administration just below the skin.

“Intravenous administration” means administration into a vein.

“Intraarterial administration” means administration into an artery.

The term “agent” includes any substance, molecule, element, compound, entity, or a combination thereof. It includes, but is not limited to, e.g., protein, polypeptide, peptide or mimetic, small organic molecule, polysaccharide, polynucleotide, and the like. It can be a natural product, a synthetic compound, or a chemical compound, or a combination of two or more substances.

“Pharmaceutical agent” means a substance that provides a therapeutic effect when administered to a subject.

“Pharmaceutical composition” means a mixture of substances suitable for administering to an individual that includes a pharmaceutical agent.

The term “pharmaceutically acceptable salt” refers to salts that retain the biological effectiveness and properties of the compounds with which they are associated and, which are not biologically or otherwise undesirable. In many cases, the compounds herein are capable of forming acid and/or base salts by virtue of the presence of phenol and/or phosphonate groups or groups similar thereto. One of ordinary skill in the art will be aware that the protonation state of any or all of these compounds may vary with pH and ionic character of the surrounding solution, and thus the present disclosure contemplates multiple charge states of each compound. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

“Solvate” refers to the compound formed by the interaction of a solvent and an EPI, a metabolite, or salt thereof. Suitable solvates are pharmaceutically acceptable solvates including hydrates.

As used herein, “Cₐ to C_{b}” or “C_{a-b}” in which “a” and “b” are integers refer to the number of carbon atoms in the specified group. That is, the group can contain from “a” to “b”, inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—.

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group of the compounds may be designated as “C₁₋₄ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, isopropyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “haloalkyl” refers to a straight- or branched-chain alkyl group having from 1 to 12 carbon atoms in the chain, substituting one or more hydrogens with halogens. Examples of haloalkyl groups include, but are not limited to, —CF₃, —CHF₂, —CH₂F, —CH₂CF₃, —CH₂CHF₂, —CH₂CH₂F, —CH₂CH₂Cl, —CH₂CF₂CF₃ and other groups that in light of the ordinary skill in the art and the teachings provided herein, would be considered equivalent to any one of the foregoing examples.

As used herein, “alkoxy” refers to the formula═OR wherein R is an alkyl as is defined above, such as “C₁₋₉ alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “polyethylene glycol” refers to the formula

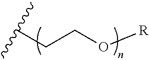

wherein n is an integer greater than one and R is a hydrogen or alkyl. The number of repeat units “n” may be indicated by referring to a number of members. Thus, for example, “2- to 5-membered polyethylene glycol” refers to n being an integer selected from two to five. In some embodiments, R is selected from methoxy, ethoxy, n-propoxy, 1-methylethoxy (isopropoxy), n-butoxy, iso-butoxy, sec-butoxy, and tert-butoxy.

As used herein, “heteroalkyl” refers to a straight or branched hydrocarbon chain containing one or more heteroatoms, that is, an element other than carbon, including but not limited to, nitrogen, oxygen and sulfur, in the chain backbone. The heteroalkyl group may have 1 to 20 carbon atoms although the present definition also covers the occurrence of the term “heteroalkyl” where no numerical range is designated. The heteroalkyl group may also be a medium size heteroalkyl having 1 to 9 carbon atoms. The heteroalkyl group could also be a lower heteroalkyl having 1 to 4 carbon atoms. In various embodiments, the heteroalkyl may have from 1 to 4 heteroatoms, from 1 to 3 heteroatoms, 1 or 2 heteroatoms, or 1 heteroatom. The heteroalkyl group of the compounds may be designated as “C₁₋₄ heteroalkyl” or similar designations. The heteroalkyl group may contain one or more heteroatoms. By way of example only, “C₁₋₄ heteroalkyl” indicates that there are one to four carbon atoms in the heteroalkyl chain and additionally one or more heteroatoms in the backbone of the chain.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆₋₁₀ aryl,” “C₆ or C₁₀ aryl,” or similar designations. Examples of aryl groups include, but are not limited to, phenyl, naphthyl, azulenyl, and anthracenyl.

As used herein, “aryloxy” and “arylthio” refers to RO- and RS-, in which R is an aryl as is defined above, such as “C₆₋₁₀ aryloxy” or “C₆₋₁₀ arylthio” and the like, including but not limited to phenyloxy.

An “aralkyl” or “arylalkyl” is an aryl group connected, as a substituent, via an alkylene group, such “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁₋₄ alkylene group).

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. In various embodiments, a heteroaryl contains from 1 to 4 heteroatoms, from 1 to 3 heteroatoms, from 1 to 2 heteroatoms, or 1 heteroatom. For example, in various embodiments, a heteroaryl contains 1 to 4 nitrogen atoms, 1 to 3 nitrogen atoms, 1 to 2 nitrogen atoms, 2 nitrogen atoms and 1 sulfur or oxygen atom, 1 nitrogen atom and 1 sulfur or oxygen atom, or 1 sulfur or oxygen atom. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C₁₋₄ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle [2.2.2]octanyl, adamantyl, and spiro[4.4]nonanyl.

A “(carbocyclyl) alkyl” is a carbocyclyl group connected, as a substituent, via an alkylene group, such as “C₄₋₁₀ (carbocyclyl) alkyl” and the like, including but not limited to, cyclopropylmethyl, cyclobutylmethyl, cyclopropylethyl, cyclopropylbutyl, cyclobutylethyl, cyclopropylisopropyl, cyclopentylmethyl, cyclopentylethyl, cyclohexylmethyl, cyclohexylethyl, cycloheptylmethyl, and the like. In some cases, the alkylene group is a lower alkylene group.

As used herein, “cycloalkyl” means a fully saturated carbocyclyl ring or ring system. Examples include cyclopropyl, cyclobutyl, cyclopentyl, and cyclohexyl.

As used herein, “cycloalkenyl” means a carbocyclyl ring or ring system having at least one double bond, wherein no ring in the ring system is aromatic. An example is cyclohexenyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations.

In various embodiments, a heterocyclyl contains from 1 to 4 heteroatoms, from 1 to 3 heteroatoms, from 1 to 2 heteroatoms, or 1 heteroatom. For example, in various embodiments, a heterocyclyl contains 1 to 4 nitrogen atoms, 1 to 3 nitrogen atoms, 1 to 2 nitrogen atoms, 2 nitrogen atoms and 1 sulfur or oxygen atom, 1 nitrogen atom and 1 sulfur or oxygen atom, or 1 sulfur or oxygen atom. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

A “(heterocyclyl) alkyl” is a heterocyclyl group connected, as a substituent, via an alkylene group. Examples include, but are not limited to, imidazolinylmethyl and indolinylethyl.

As used herein, “acyl” refers to —C(═O)R, wherein R is hydrogen, C₁₋₆ alkyl, C₂-6 alkenyl, C₂₋₆ alkynyl, C₃₋₇ carbocyclyl, aryl, 5-10 membered heteroaryl, and 5-10 membered heterocyclyl, as defined herein. Non-limiting examples include formyl, acetyl, propanoyl, benzoyl, and acryl.

An “O-carboxy” group refers to a “—OC(═O) R” group in which R is selected from hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein.

A “C-carboxy” group refers to a “—C(═O) OR” group in which R is selected from hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. A non-limiting example includes carboxyl (i.e., —C(═O) OH).

A “cyano” group refers to a “—CN” group.

A “cyanato” group refers to an “—OCN” group.

An “isocyanato” group refers to a “—NCO” group.

A “thiocyanato” group refers to a “—SCN” group.

An “isothiocyanato” group refers to an “—NCS” group.

A “sulfinyl” group refers to an “—S(═O) R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “sulfonyl” group refers to an “—SO₂R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “S-sulfonamido” group refers to a “—SO₂NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-sulfonamido” group refers to a “—N(R_{A}) SO₂R_{B}” group in which R_{A} and R_{b} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “O-carbamyl” group refers to a “—OC(═O) NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-carbamyl” group refers to an “—N(R_{A}) OC(═O) R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “O-thiocarbamyl” group refers to a “—OC(═S) NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-thiocarbamyl” group refers to an “—N(R_{A}) OC(=S) R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “C-amido” group refers to a “—C(═O) NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-amido” group refers to a “—N(R_{A}) C(═O) R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “amino” group refers to a “—NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “aminoalkyl” group refers to an amino group connected via an alkylene group.

An “alkoxyalkyl” group refers to an alkoxy group connected via an alkylene group, such as a “C₂₋₈ alkoxyalkyl” and the like.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more subsitutents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 5-10 membered heterocyclyl (optionally substituted with halo, C₁-C₆ alkyl, C₁-C₆ alkoxy, C₁-C₆ haloalkyl, and C₁-C₆ haloalkoxy), 5-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl (C₁-C₆) alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl (C₁-C₆) alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy (C₁-C₆) alkyl (i.e., ether), aryloxy, sulfhydryl (mercapto), halo (C₁-C₆) alkyl (e.g., —CF₃), halo (C₁-C₆) alkoxy (e.g., —OCF₃), C₁-C₆ alkylthio, arylthio, amino, amino (C₁-C₆) alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Wherever a group is described as “optionally substituted” that group can be substituted with the above substituents.

In some embodiments, substituted group(s) is (are) substituted with one or more substituent(s) individually and independently selected from C₁-C₄ alkyl, amino, hydroxy, and halogen.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

When two R groups are said to form a ring (e.g., a carbocyclyl, heterocyclyl, aryl, or heteroaryl ring) “together with the atom to which they are attached,” it is meant that the collective unit of the atom and the two R groups are the recited ring. The ring is not otherwise limited by the definition of each R group when taken individually. For example, when the following substructure is present:

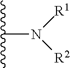

and R¹ and R² are defined as selected from the group consisting of hydrogen and alkyl, or R¹ and R² together with the nitrogen to which they are attached form a heterocyclyl, it is meant that R¹ and R² can be selected from hydrogen or alkyl, or alternatively, the substructure has structure:

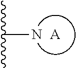

where ring A is a heterocyclyl ring containing the depicted nitrogen.

Similarly, when two “adjacent” R groups are said to form a ring “together with the atoms to which they are attached,” it is meant that the collective unit of the atoms, intervening bonds, and the two R groups are the recited ring. For example, when the following substructure is present:

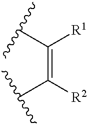

and R¹ and R² are defined as selected from the group consisting of hydrogen and alkyl, or R¹ and R² together with the atoms to which they are attached form an aryl or carbocyclyl, it is meant that R¹ and R² can be selected from hydrogen or alkyl, or alternatively, the substructure has structure:

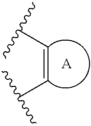

where A is an aryl ring or a carbocyclyl containing the depicted double bond.

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

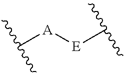

includes the substituent being oriented such that the A is attached at the leftmost attachment point of the molecule as well as the case in which A is attached at the rightmost attachment point of the molecule.

Isotopes may be present in the compounds described. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

### Combinations With Additional Pharmaceutical Agents

The compounds of Formula (I) presented herein may be administered in combination with one or more additional pharmaceutical agents. In some embodiments, the compounds described above may be administered in combination with one additional pharmaceutical agent. In some embodiments, the compounds described above may be administered in combination with two additional pharmaceutical agents. In some embodiments, the compounds described above may be administered in combination with three or more additional pharmaceutical agents.

In some embodiments, the compounds of Formula (I) presented herein may be administered simultaneously with one or more additional pharmaceutical agents. In other embodiments, the compounds of the present disclosure may be administered sequentially with one or more additional pharmaceutical agents.

### Aminoglycosides

In some embodiments, the compounds of Formula (I) presented herein may be administered in combination with an aminoglycoside. Aminoglycosides are compounds that have an amino-modified glycoside. Such compounds have medicinal applications, e.g., as antibiotics and by promoting readthrough of premature termination codons (PTC). In some specific embodiments, aminoglycosides may increase ribosomal readthrough of mRNA transcripts carrying a PTC mutation of the cystic fibrosis CFTR channel. Aminoglycosides include, but are not limited to, kanamycin A, amikacin, tobramycin, dibekacin, gentamicin, geneticin, sisomicin, netilmicin, neomycin B, neomycin C, paromomycin, streptomycin, plazomicin, tobramycin, and

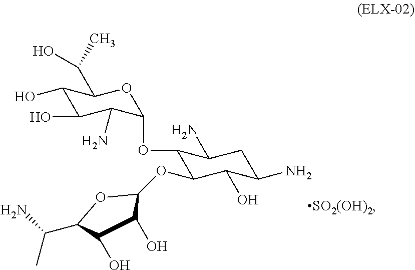

pharmaceutically acceptable salts thereof. In some embodiments, the aminoglycoside may be geneticin, ELX-02, or paromomycin.

Enhancement of premature termination codon readthrough using the readthrough modulator compounds disclosed herein may be observed when, e.g., co-administering the readthrough modular compounds with an aminoglycoside. For instance, co-administration of the readthrough modulator compounds disclosed herein with an aminoglycoside can result in enhancement of premature termination codon readthrough even with administration of a submaximal effective administration of an aminoglycoside. This has the benefit of maximizing enhancement of PTC mutant readthrough while limiting the toxic side effects of aminoglycosides. Without being bound by any particular theory, the readthrough modulator compounds disclosed herein may increase the potency and/or effect of the aminoglycoside. In some embodiments, administration of the readthrough modulator compounds disclosed herein may reduce the concentration of aminoglycoside necessary to achieve maximal effectiveness by 5%, 10%, 15%, 20%, 25%, 30%, 35%, 40%, 45%, 50%, 55%, 60%, 65%, 70%, 75%, 80%, 85%, 90%, or more as compared to the maximal effective concentration of aminoglycoside when administered alone, or within a range defined by any of the aforementioned percentages. Thus, for example, some embodiments include co-administering a compound of Formula (I) with an aminoglycoside, wherein the amount of aminoglycoside is between 5% and 90%, between 5% and 80%, between 5% and 60%, between 5% and 40%, or between 10% and 30% of the maximally effective amount of aminglycoside. In some embodiments, the aminoglycoside may be G418, ELX-02, or any aminoglycoside disclosed herein.

eRF3 Modulators

In some embodiments, the compounds of Formula (I) presented herein may be administered in combination with one or more compounds that reduce levels of eRF3 protein in a subject. The eRF3 protein plays a role in translation termination and post-termination events. See, e.g., Baradaran-Heravi et al. Nucleic Acids Research 2021, 1-17. In some embodiments, the compounds of Formula (I) presented herein may be administered in combination with both an aminoglycoside and an eRF3 modulator.

Enhancement of premature termination codon readthrough using the readthrough modulator compounds disclosed herein may also be observed when co-administering the readthrough modular compounds with an aminoglycoside and an eRF3 modulator,. e.g., such as a cereblon E3 ligase modulator. Co-administration of the readthrough modulator compounds disclosed herein with an aminoglycoside and an eRF3 modulator can result in enhancement of premature termination codon readthrough even with administration of a submaximal effective administration of an aminoglycoside. In some embodiments, administration of the readthrough modulator compounds disclosed herein in combination with an eRF3 modulatory may reduce the concentration of aminoglycoside neccesary to achieve maximal effectiveness by 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or more as compared to the maximal effective concentration of aminoglycoside when administered alone. Thus, for example, some embodiments include co-administering a compound of Formula (I) with an aminoglycoside and an eRF3 modulator, wherein the amount of aminoglycoside is between 5% and 90%, between 5% and 80%, between 5% and 60%, between 5% and 40%, or between 10% and 30% of the maximally effective amount of aminglycoside. In some embodiments, the aminoglycoside may be G418, ELX-02, or any aminoglycoside disclosed herein. In some embodiments, the eRF3 modulator may be a cereblon E3 ligase modulator disclosed herein.

Compounds that modulate eRF3 levels in a subject include cereblon E3 ligase modulators. Cereblon E3 ligase modulators may be used for targeted protein degradation in a subject. In some embodiments, the cereblon E3 ligase modulator may be a compound having the formula:

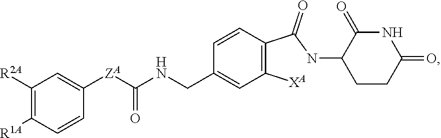

or a pharmaceutically acceptable salt thereof, wherein:
X^{A} is CH₂ or C═O;
Z^{A} is: (CH₂) m or NH;
m is 0 or 1;
R^{1A} is halogen or (C₁-C₆) alkyl optionally substituted with one or more halogen; and
R^{2A} is hydrogen, halogen, or (C₁-C₆) alkyl optionally substituted with one or more halogen,

In some embodiments, the cereblon E3 ligase modulator may be a compound having the formula:

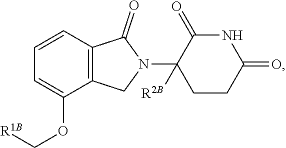

or a pharmaceutically acceptable salt thereof, wherein:
X^{B} is C═O or CH₂;
R^{1B} is —Y^{B}—R^{3B}.
R^{2B} is H or (C₁-C₅) alkyl;
Y^{B} is: 6 to 10 membered aryl optionally substituted with one or more halogen;
R^{3B} is:
   (i) —(CH₂)-aryl, —O—(CH₂)ₙ-aryl or —(CH₂), —O-aryl, wherein the aryl is optionally substituted with one or more: (C₁-C₆) alkyl, itself optionally substituted with one or more halogen; (C₁-C₆) alkoxy, itself substituted with one or more halogen; oxo; amino; carboxyl; cyano; hydroxyl; halogen; 6 to 10 membered aryl or heteroaryl, each optionally substituted with one or more (C₁-C₆) alkyl, (C₁-C₆) alkoxy or halogen; —CONH₂; or —COO—(C₁-C₆) alkyl, wherein the alkyl may be optionally substituted with one or more halogen;
   (ii) —(CH₂), -heterocycle, —O—(CH₂)-heterocycle or —(CH₂), —O-heterocycle, wherein the heterocycle is optionally substituted with one or more: (C₁-C₅) alkyl, itself optionally substituted with one or more halogen; (C₁-C₆) alkoxy, itself substituted with one or more halogen; oxo; amino; carboxyl; cyano; hydroxyl; halogen; 6 to 10 membered aryl or heteroaryl, each optionally substituted with one or more (C₁-C₆) alkyl, (C₁-C₆) alkoxy or halogen; —CONH₂; or —COO—(C₁-C₆) alkyl, wherein the alkyl may be optionally substituted with one or more halogen; or
   (iii) —(CH₂)-heteroaryl, —O—(CH₂)-heteroaryl or —(CH₂)ₐ—O-heteroaryl, wherein the heteroaryl is optionally substituted with one or more: (C₁-C₆) alkyl, itself optionally substituted with one or more halogen; (C₁-C₆) alkoxy, itself substituted with one or more halogen; oxo; amino; carboxyl; cyano; hydroxyl; halogen; 6 to 10 membered aryl or beteroaryl, each optionally substituted with one or more (C₁-C₆) alkyl, (C₁-C₆) alkoxy or halogen; —CONH₂; or —COO—(C₁-C₆) alkyl, wherein the alkyl may be optionally substituted with one or more halogen; and
n is 0, 1, 2 or 3.

In some embodiments, the cereblon E3 ligase modulator may be a compound having the formula:

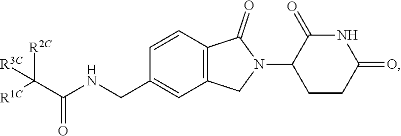

or a pharmaceutically acceptable salt thereof, wherein:
R^{1C} is optionally substituted cycloalkyl, optionally substituted aryl, optionally substituted heteroaryl or optionally substituted heterocyclyl;
R^{2C} and R^{3C} are each halo;
where the substituents on R^{1C}, when present, are one to three groups QC, where each QC is independently (C₁-C₆)alkyl, halo, (C₁-C₆)haloalkyl, (C₁-C₆)alkoxy-(C₁-C₆)alkyl, oxo, hydroxyl, (C₁-C₆)alkoxy, optionally substituted (C₃-C₆) cycloalkyl, optionally substituted cycloalkylalkyl, optionally substituted heterocyclyl, optionally substituted heterocyclylalkyl, optionally substituted aryl, optionally substituted heteroaryl, —R^{4C}OR^{5C}, —R^{4C}OR^{5C}—R^{4C}OR^{5C}, —R^{4C}N(R^{6C})(R^{7C}), —R^{4C}SR^{5C}, —R^{4C}OR^{4C}N(R^{6C})(R^{7C}), R^{4C}OR^{4C}C(J^{C})N(R^{6C})(R^{7C}), —C(J^{C}) R^{9C} or R^{4C}S(O)ₜR^{8C};
each R^{4C} is independently alkylene, alkenylene or a direct bond;
each R^{5C} is independently hydrogen, (C₁-C₆) alkyl, (C₁-C₆) haloalkyl, (C₁-C₆) hydroxyalkyl, (C₁-C₆) alkoxy-(C₁-C₆) alkyl, (C₃-Cₐ) cycloalkyl, aryl, heteroaryl, heterocyclyl or heterocyclylalkyl, where alkyl, haloalkyl, hydroxyalkyl, alkoxyalkyl, cycloalkyl, aryl, heteroaryl, heterocyclyl or heterocyclylalkyl groups in R^{5C} are each independently optionally substituted with 1-3 QC′ groups, where each QC′ is independently alkyl, haloalkyl or halo;
R^{6C} and R^{7C} are selected as follows:
i) R^{6C} and R^{7C} are each independently hydrogen or (C₁-C₆) alkyl; or
ii) R^{6C} and R^{7C} together with the nitrogen atom on which they are substituted form a 5 or 6-membered heterocyclyl or heteroaryl ring, optionally substituted with one or two halo, (C₁-C₅) alkyl or haloalkyl;
R^{8C} is (C₁-C₆) alkyl, (C₁-C₆) haloalkyl, or (C₁-C₆) hydroxyalkyl;
R^{9C} is (C₁-C₅) alkyl or aryl;
J^{C} is O or S; and
t is 1 or 2.

In some embodiments, the cereblon E3 ligase modulator may be a compound having the formula:

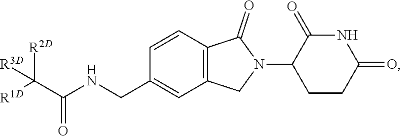

or a pharmaceutically acceptable, wherein:
R^{1D} is optionally substituted (C₃-Cₐ) cycloalkyl, optionally substituted aryl, optionally substituted heteroaryl or optionally substituted heterocyclyl;
R^{2D} and R^{3D} are each halo;
where the substituents on R^{1D}, when present, are one to three groups Q^{D}, where each Q^{D} is independently (C₁-C₆) alkyl, halo, (C₁-C₆) haloalkyl, (C₁-C₆) alkoxy-(C₁-C₆) alkyl, oxo, hydroxyl, (C₁-C₆) alkoxy, optionally substituted (C₃-C₈) cycloalkyl, optionally substituted cycloalkylalkyl, optionally substituted heterocyclyl, optionally substituted heterocyclylalkyl, optionally substituted aryl, optionally substituted heteroaryl, —R^{4D}OR^{5D}, —R^{4D} OR^{5D}—R^{4D} OR^{5D}, —R^{4D} N(R^{6D}) (R^{7D}), —R^{4D} SR^{5D}, —R^{4D} OR^{4D} N(R^{6D}) (R^{7D}),
R^{4D} OR^{4D} C(J^{D})N(R^{6D})(R^{7D}), —C(J^{D})R^{9D} or R^{4D} S(O)WR^{8D};
each R^{4D} is independently alkylene, alkenylene or a direct bond;
each R^{5D} is independently hydrogen, (C₁-C₆) alkyl, (C₁-C₅) haloalkyl, hydroxy (C₁-C₆) alkyl, (C₁-C₆) alkoxy-(C₁-C₆) alkyl, (C₃-C₈) cycloalkyl, aryl, heteroaryl, heterocyclyl or heterocyclylalkyl, where alkyl, haloalkyl, hydroxyalkyl, alkoxyalkyl, cycloalkyl, aryl, heteroaryl, heterocyclyl or heterocyclylalkyl groups in R^{5D} are each independently optionally substituted with 1-3 Q^{D′} groups, where each Q^{D′} is independently (C₁-C₆) alkyl, (C₁-C₆) haloalkyl or halo;
R^{6D} and R^{7D} are selected as follows:
i) R^{6D} and R^{7D} are each independently hydrogen or (C₁-C₆) alkyl; or
ii) R^{6D} and R^{7D} together with the nitrogen atom on which they are substituted form a 5 or 6-membered heterocyclyl or heteroaryl ring, optionally substituted with one or two halo, (C₁-C₅) alkyl or (C₁-C₆) haloalkyl;
R^{8D} is (C₁-C₆) alkyl, (C₁-C₆) haloalkyl, or hydroxy (C₁-C₆) alkyl;
R^{9D} is (C₁-C₆) alkyl or aryl;
J^{D} is O or S; and
w is 1 or 2.

In some embodiments, the cereblon E3 ligase modulator may be a compound selected from the group consisting of

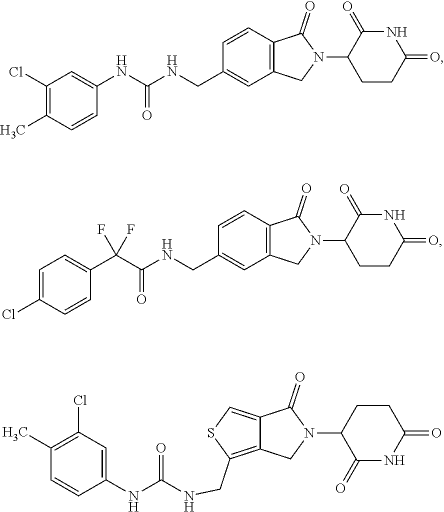 [SMILES: Cc1ccc(NC(=O)NCc2ccc3c(c2)CN(C2CCC(=O)NC2=O)C3=O)cc1Cl ; O=C1CCC(N2Cc3cc(CNC(=O)C(F)(F)c4ccc(Cl)cc4)ccc3C2=O)C(=O)N1 ; Cc1ccc(NC(=O)NCc2scc3c2CN(C2CCC(=O)NC2=O)C3=O)cc1Cl]

and pharmaceutically acceptable salts thereof.

### Pharmaceutical Compositions

The compounds of Formula (I) as described above and/or the additional pharmaceutical agents described above can be formulated into pharmaceutical compositions for use in treatment of the conditions described herein. Standard pharmaceutical formulation techniques are used, such as those disclosed in Remington's The Science and Practice of Pharmacy, 21st Ed., Lippincott Williams & Wilkins (2005), incorporated herein by reference in its entirety. Accordingly, some embodiments include pharmaceutical compositions comprising: (a) a safe and therapeutically effective amount of a compound described herein, or pharmaceutically acceptable salts thereof; and (b) a pharmaceutically acceptable carrier, diluent, excipient or combination thereof.

In some embodiments, the compounds of Formula (I) provided herein and the one or more additional pharmaceutical agents provided herein may be formulated into a single pharmaceutical composition for use in treatment of the conditions described herein. In some embodiments, a formulation comprising the compounds of Formula (I) provided herein may be administered in combination with one or more additional pharmaceutical agents provided herein or a pharmaceutical composition comprising one or more additional pharmaceutical agents provided herein. In some embodiments, the pharmaceutical composition may comprise one or more compounds of Formula (I) provided herein and one or more aminoglycosides. In other embodiments, the pharmaceutical composition may comprise one or more compounds of Formula (I) provided herein and one or more eRF3 modulators. In some embodiments, the pharmaceutical composition may comprise one or more compounds of Formula (I) provided herein, one or more aminoglycosides, and one or more eRF3 modulators. In some embodiments, the pharmaceutical composition may comprise one or more aminoglycosides and one or more eRF3 modulators. In some embodiments, the eRF3 modulator may be a cereblon E3 ligase modulator.

The term “pharmaceutically acceptable carrier” or “pharmaceutically acceptable excipient” includes any and all solvents, diluents, emulsifiers, binders, buffers, dispersion media, coatings, antibacterial and antifungal agents, isotonic and absorption delaying agents and the like, or any other such compound as is known by those of skill in the art to be useful in preparing pharmaceutical formulations. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. In addition, various adjuvants such as are commonly used in the art may be included. These and other such compounds are described in the literature, e.g., in the Merck Index, Merck & Company, Rahway, NJ. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such as sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is determined by the way the compound is to be administered.

The compositions described herein are preferably provided in unit dosage form. As used herein, a “unit dosage form” is a composition containing an amount of a compound that is suitable for administration to a subject, in a single dose, according to good medical practice. The preparation of a single or unit dosage form however, does not imply that the dosage form is administered once per day or once per course of therapy. A unit dosage form may comprise a single daily dose or a fractional sub-dose wherein several unit dosage forms are to be administered over the course of a day in order to complete a daily dose. According to the present disclosure, a unit dosage form may be given more or less often that once daily, and may be administered more than once during a course of therapy. Such dosage forms may be administered in any manner consistent with their formulation, including orally, parenterally, and may be administered as an infusion over a period of time (e.g., from about 30 minutes to about 2-6 hours). While single administrations are specifically contemplated, the compositions administered according to the methods described herein may also be administered as a continuous infusion or via an implantable infusion pump.

The methods as described herein may utilize any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions include compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropes, surface-active agents, and encapsulating substances. Optional pharmaceutically-active materials may be included, which do not substantially interfere with the activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carriers suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid, microcrystalline cellulose, carboxymethyl cellulose, and talc. Tablets may also comprise solubilizers or emulsifiers, such as poloxamers, cremophor/Kolliphor®/Lutrol®, methylcellulose, hydroxypropylmethylcellulose, or others as are known in the art. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which can be readily made by a person skilled in the art.

Peroral (PO) compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort may be maximized as much as possible, although sometimes formulation considerations (e.g. drug stability) may necessitate less than optimal comfort. In the case that comfort cannot be maximized, the liquid may be formulated such that the liquid is tolerable to the patient for topical ophthalmic use. Additionally, an ophthalmically acceptable liquid may either be packaged for single use, or contain a preservative to prevent contamination over multiple uses.

For ophthalmic application, solutions or medicaments are often prepared using a physiological saline solution as a major vehicle. Ophthalmic solutions may preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

Ophthalmically acceptable antioxidants include, but are not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For topical use, including for transdermal administration, creams, ointments, gels, solutions or suspensions, etc., containing the compound disclosed herein are employed. Topical formulations may generally be comprised of a pharmaceutical carrier, co-solvent, emulsifier, penetration enhancer, preservative system, and emollient.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J. Pharm. Sci. Tech. 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The actual unit dose of the readthrough modulator compounds described herein and/or additional pharmaceutical agents described herein depends on the specific compound, and on the condition to be treated. In some embodiments, the dose may be from about 0.01 mg/kg to about 120 mg/kg or more of body weight, from about 0.05 mg/kg or less to about 70 mg/kg, from about 0.1 mg/kg to about 50 mg/kg of body weight, from about 1.0 mg/kg to about 10 mg/kg of body weight, from about 5.0 mg/kg to about 10 mg/kg of body weight, or from about 10.0 mg/kg to about 20.0 mg/kg of body weight. In some embodiments, the dose may be less than 100 mg/kg, 90 mg/kg, 80 mg/kg, 70 mg/kg, 60 mg/kg, 50 mg/kg, 40 mg/kg, 30 mg/kg, 25 mg/kg, 20 mg/kg, 10 mg/kg, 7.5 mg/kg, 6 mg/kg, 5 mg/kg, 4 mg/kg, 3 mg/kg, 2.5 mg/kg, 1 mg/kg, 0.5 mg/kg, 0.1 mg/kg, 0.05 mg/kg or 0.005 mg/kg of body weight. In some embodiments, the actual unit dose is 0.05, 0.07, 0.1, 0.3, 1.0, 3.0, 5.0, 10.0 or 25.0 mg/kg of body weight, or a range between any two of these values. Thus, for administration to a 70 kg person, the dosage range would be from about 0.1 mg to 70 mg, from about 1 mg to about 50 mg, from about 0.5 mg to about 10 mg, from about 1 mg to about 10 mg, from about 2.5 mg to about 30 mg, from about 35 mg or less to about 700 mg or more, from about 7 mg to about 600 mg, from about 10 mg to about 500 mg, or from about 20 mg to about 300 mg, or from about 200 mg to about 2000 mg. In some embodiments, the actual unit dose is 0.1 mg. In some embodiments, the actual unit dose is 0.5 mg. In some embodiments, the actual unit dose is 1 mg. In some embodiments, the actual unit dose is 1.5 mg. In some embodiments, the actual unit dose is 2 mg. In some embodiments, the actual unit dose is 2.5 mg. In some embodiments, the actual unit dose is 3 mg. In some embodiments, the actual unit dose is 3.5 mg. In some embodiments, the actual unit dose is 4 mg. In some embodiments, the actual unit dose is 4.5 mg. In some embodiments, the actual unit dose is 5 mg. In some embodiments the actual unit dose is 10 mg. In some embodiments, the actual unit dose is 25 mg. In some embodiments, the actual unit dose is 250 mg or less. In some embodiments, the actual unit dose is 100 mg or less. In some embodiments, the actual unit dose is 70 mg or less.

In some embodiments, the readthrough modulator compounds described herein may be administered at a dose in the range of about 1-50 mg/m2 of the body surface area. In some embodiments, the readthrough modulator compounds described herein may be administered at a dose in the range of about 1-2, 1-3, 1-4, 1-5, 1-6, 1-7, 1-8, 1-9, 1-10, 1-11, 1-12, 1-13, 1-13.75, 1-14, 1-15, 1-16, 1-17, 1-18, 1-19, 1-20, 1-22.5, 1-25, 1-27.5, 1-30, 1.5-2, 1.5-3, 1.5-4, 1.5-5, 1.5-6, 1.5-7, 1.5-8, 1.5-9, 1.5-10, 1.5-11, 1.5-12, 1.5-13, 1.5-13.75, 1.5-14, 1.5-15, 1.5-16, 1.5-17, 1.5-18, 1.5-19, 1.5-20, 1.5-22.5, 1.5-25, 1.5-27.5, 1.5-30, 2.5-2, 2.5-3, 2.5-4, 2.5-5, 2.5-6, 2.5-7, 2.5-8, 2.5-9, 2.5-10, 2.5-11, 2.5-12, 2.5-13, 2.5-13.75, 2.5-14, 2.5-15, 2.5-16, 2.5-17, 2.5-18, 2.5-19, 2.5-20, 2.5-22.5, 2.5-25, 2.5-27.5, 2.5-30, 2.5-7.5, 3-4, 3-5, 3-6, 3-7, 3-8, 3-9, 3-10, 3-11, 3-12, 3-13, 3-13.75, 3-14, 3-15, 3-16, 3-17, 3-18, 3-19, 3-20, 3-22.5, 3-25, 3-27.5, 3-30, 3.5-6.5, 3.5-13.75, 3.5-15, 2.5-17.5, 4-5, 4-6, 4-7, 4-8, 4-9, 4-10, 4-11, 4-12, 4-13, 4-13.75, 4-14, 4-15, 4-16, 4-17, 4-18, 4-19, 4-20, 4-22.5, 4-25, 4-27.5, 4-30, 5-6, 5-7, 5-8, 5-9, 5-10, 5-11, 5-12, 5-13, 5-13.75, 5-14, 5-15, 5-16, 5-17, 5-18, 5-19, 5-20, 5-22.5, 5-25, 5-27.5, 5-30, 6-7, 6-8, 6-9, 6-10, 6-11, 6-12, 6-13, 6-13.75, 6-14, 6-15, 6-16, 6-17, 6-18, 6-19, 6-20, 6-22.5, 6-25, 6-27.5, 6-30, 7-8, 7-9, 7-10, 7-11, 7-12, 7-13, 7-13.75, 7-14, 7-15, 7-16, 7-17, 7-18, 7-19, 7-20, 7-22.5, 7-25, 7-27.5, 7-30, 7.5-12.5, 7.5-13.5, 7.5-15, 8-9, 8-10, 8-11, 8-12, 8-13, 8-13.75, 8-14, 8-15, 8-16, 8-17, 8-18, 8-19, 8-20, 8-22.5, 8-25, 8-27.5, 8-30, 9-10, 9-11, 9-12, 9-13, 9-13.75, 9-14, 9-15, 9-16, 9-17, 9-18, 9-19, 9-20, 9-22.5, 9-25, 9-27.5, 9-30, 10-11, 10-12, 10-13, 10-13.75, 10-14, 10-15, 10-16, 10-17, 10-18, 10-19, 10-20, 10-22.5, 10-25, 10-27.5, 10-30, 11.5-15.5, 12.5-14.5, 7.5-22.5, 8.5-32.5, 9.5-15.5, 15.5-24.5, 5-35, 17.5-22.5, 22.5-32.5, 25-35, 25.5-24.5, 27.5-32.5, 2-20, 2.5-22.5, or 9.5-21.5 mg/m², of the body surface area. In some embodiments, the readthrough modulator compounds described herein may be administered at a dose of about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40 mg/m² of the body surface area. In some embodiments, the readthrough modulator compounds described herein may be administered at a dose less than about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40 mg/m² of the body surface area. In some embodiments, the readthrough modulator compounds described herein may be administered at a dose greater than about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40, 41, 42, 43, 44, 45, 46, 47, 48, 49, 50 mg/m² of the body surface area.

In some embodiments, the readthrough modulator compound dose is about 0.1 mg-100 mg, 0.1 mg-50 mg, 0.1 mg-20 mg, 0.1 mg-10 mg, 0.5 mg-100 mg, 0.5 mg-50 mg, 0.5 mg-20 mg, 0.5 mg-10 mg, 1 mg-100 mg, 1 mg-50 mg, 1 mg-20 mg, 1 mg-10 mg, 2.5 mg-50 mg, 2.5 mg-20 mg, 2.5 mg-10 mg, or about 2.5 mg-5 mg. In some embodiments, the readthrough modulator compound dose is about 5 mg-300 mg, 5 mg-200 mg, 7.5 mg-200 mg, 10 mg-100 mg, 15 mg-100 mg, 20 mg-100 mg, 30 mg-100 mg, 40 mg-100 mg, 10 mg-80 mg, 15 mg-80 mg, 20 mg-80 mg, 30 mg-80 mg, 40 mg-80 mg, 10 mg-60 mg, 15 mg-60 mg, 20 mg-60 mg, 30 mg-60 mg, or about 40 mg-60 mg. In some embodiments, the readthrough modulator compound administered is about 20 mg-60 mg, 27 mg-60 mg, 20 mg-45 mg, or 27 mg-45 mg. In some embodiments, the readthrough modulator compound administered is about 5 mg-7.5 mg, 5 mg-9 mg, 5 mg-10 mg, 5 mg-12 mg, 5 mg-14 mg, 5 mg-15 mg, 5 mg-16 mg, 5 mg-18 mg, 5 mg-20 mg, 5 mg-22 mg, 5 mg-24 mg, 5 mg-26 mg, 5 mg-28 mg, 5 mg-30 mg, 5 mg-32 mg, 5 mg-34 mg, 5 mg-36 mg, 5 mg-38 mg, 5 mg-40 mg, 5 mg-42 mg, 5 mg-44 mg, 5 mg-46 mg, 5 mg-48 mg, 5 mg-50 mg, 5 mg-52 mg, 5 mg-54 mg, 5 mg-56 mg, 5 mg-58 mg, 5 mg-60 mg, 7 mg-7.7 mg, 7 mg-9 mg, 7 mg-10 mg, 7 mg-12 mg, 7 mg-14 mg, 7 mg-15 mg, 7 mg-16 mg, 7 mg-18 mg, 7 mg-20 mg, 7 mg-22 mg, 7 mg-24 mg, 7 mg-26 mg, 7 mg-28 mg, 7 mg-30 mg, 7 mg-32 mg, 7 mg-34 mg, 7 mg-36 mg, 7 mg-38 mg, 7 mg-40 mg, 7 mg-42 mg, 7 mg-44 mg, 7 mg-46 mg, 7 mg-48 mg, 7 mg-50 mg, 7 mg-52 mg, 7 mg-54 mg, 7 mg-56 mg, 7 mg-58 mg, 7 mg-60 mg, 9 mg-10 mg, 9 mg-12 mg, 9 mg-14 mg, 9 mg-15 mg, 9 mg-16 mg, 9 mg-18 mg, 9 mg-20 mg, 9 mg-22 mg, 9 mg-24 mg, 9 mg-26 mg, 9 mg-28 mg, 9 mg-30 mg, 9 mg-32 mg, 9 mg-34 mg, 9 mg-36 mg, 9 mg-38 mg, 9 mg-40 mg, 9 mg-42 mg, 9 mg-44 mg, 9 mg-46 mg, 9 mg-48 mg, 9 mg-50 mg, 9 mg-52 mg, 9 mg-54 mg, 9 mg-56 mg, 9 mg-58 mg, 9 mg-60 mg, 10 mg-12 mg, 10 mg-14 mg, 10 mg-15 mg, 10 mg-16 mg, 10 mg-18 mg, 10 mg-20 mg, 10 mg-22 mg, 10 mg-24 mg, 10 mg-26 mg, 10 mg-28 mg, 10 mg-30 mg, 10 mg-32 mg, 10 mg-34 mg, 10 mg-36 mg, 10 mg-38 mg, 10 mg-40 mg, 10 mg-42 mg, 10 mg-44 mg, 10 mg-46 mg, 10 mg-48 mg, 10 mg-50 mg, 10 mg-52 mg, 10 mg-54 mg, 10 mg-56 mg, 10 mg-58 mg, 10 mg-60 mg, 12 mg-14 mg, 12 mg-15 mg, 12 mg-16 mg, 12 mg-18 mg, 12 mg-20 mg, 12 mg-22 mg, 12 mg-24 mg, 12 mg-26 mg, 12 mg-28 mg, 12 mg-30 mg, 12 mg-32 mg, 12 mg-34 mg, 12 mg-36 mg, 12 mg-38 mg, 12 mg-40 mg, 12 mg-42 mg, 12 mg-44 mg, 12 mg-46 mg, 12 mg-48 mg, 12 mg-50 mg, 12 mg-52 mg, 12 mg-54 mg, 12 mg-56 mg, 12 mg-58 mg, 12 mg-60 mg, 15 mg-16 mg, 15 mg-18 mg, 15 mg-20 mg, 15 mg-22 mg, 15 mg-24 mg, 15 mg-26 mg, 15 mg-28 mg, 15 mg-30 mg, 15 mg-32 mg, 15 mg-34 mg, 15 mg-36 mg, 15 mg-38 mg, 15 mg-40 mg, 15 mg-42 mg, 15 mg-44 mg, 15 mg-46 mg, 15 mg-48 mg, 15 mg-50 mg, 15 mg-52 mg, 15 mg-54 mg, 15 mg-56 mg, 15 mg-58 mg, 15 mg-60 mg, 17 mg-18 mg, 17 mg-20 mg, 17 mg-22 mg, 17 mg-24 mg, 17 mg-26 mg, 17 mg-28 mg, 17 mg-30 mg, 17 mg-32 mg, 17 mg-34 mg, 17 mg-36 mg, 17 mg-38 mg, 17 mg-40 mg, 17 mg-42 mg, 17 mg-44 mg, 17 mg-46 mg, 17 mg-48 mg, 17 mg-50 mg, 17 mg-52 mg, 17 mg-54 mg, 17 mg-56 mg, 17 mg-58 mg, 17 mg-60 mg, 20 mg-22 mg, 20 mg-24 mg, 20 mg-26 mg, 20 mg-28 mg, 20 mg-30 mg, 20 mg-32 mg, 20 mg-34 mg, 20 mg-36 mg, 20 mg-38 mg, 20 mg-40 mg, 20 mg-42 mg, 20 mg-44 mg, 20 mg-46 mg, 20 mg-48 mg, 20 mg-50 mg, 20 mg-52 mg, 20 mg-54 mg, 20 mg-56 mg, 20 mg-58 mg, 20 mg-60 mg, 22 mg-24 mg, 22 mg-26 mg, 22 mg-28 mg, 22 mg-30 mg, 22 mg-32 mg, 22 mg-34 mg, 22 mg-36 mg, 22 mg-38 mg, 22 mg-40 mg, 22 mg-42 mg, 22 mg-44 mg, 22 mg-46 mg, 22 mg-48 mg, 22 mg-50 mg, 22 mg-52 mg, 22 mg-54 mg, 22 mg-56 mg, 22 mg-58 mg, 22 mg-60 mg, 25 mg-26 mg, 25 mg-28 mg, 25 mg-30 mg, 25 mg-32 mg, 25 mg-34 mg, 25 mg-36 mg, 25 mg-38 mg, 25 mg-40 mg, 25 mg-42 mg, 25 mg-44 mg, 25 mg-46 mg, 25 mg-48 mg, 25 mg-50 mg, 25 mg-52 mg, 25 mg-54 mg, 25 mg-56 mg, 25 mg-58 mg, 25 mg-60 mg, 27 mg-28 mg, 27 mg-30 mg, 27 mg-32 mg, 27 mg-34 mg, 27 mg-36 mg, 27 mg-38 mg, 27 mg-40 mg, 27 mg-42 mg, 27 mg-44 mg, 27 mg-46 mg, 27 mg-48 mg, 27 mg-50 mg, 27 mg-52 mg, 27 mg-54 mg, 27 mg-56 mg, 27 mg-58 mg, 27 mg-60 mg, 30 mg-32 mg, 30 mg-34 mg, 30 mg-36 mg, 30 mg-38 mg, 30 mg-40 mg, 30 mg-42 mg, 30 mg-44 mg, 30 mg-46 mg, 30 mg-48 mg, 30 mg-50 mg, 30 mg-52 mg, 30 mg-54 mg, 30 mg-56 mg, 30 mg-58 mg, 30 mg-60 mg, 33 mg-34 mg, 33 mg-36 mg, 33 mg-38 mg, 33 mg-40 mg, 33 mg-42 mg, 33 mg-44 mg, 33 mg-46 mg, 33 mg-48 mg, 33 mg-50 mg, 33 mg-52 mg, 33 mg-54 mg, 33 mg-56 mg, 33 mg-58 mg, 33 mg-60 mg, 36 mg-38 mg, 36 mg-40 mg, 36 mg-42 mg, 36 mg-44 mg, 36 mg-46 mg, 36 mg-48 mg, 36 mg-50 mg, 36 mg-52 mg, 36 mg-54 mg, 36 mg-56 mg, 36 mg-58 mg, 36 mg-60 mg, 40 mg-42 mg, 40 mg-44 mg, 40 mg-46 mg, 40 mg-48 mg, 40 mg-50 mg, 40 mg-52 mg, 40 mg-54 mg, 40 mg-56 mg, 40 mg-58 mg, 40 mg-60 mg, 43 mg-46 mg, 43 mg-48 mg, 43 mg-50 mg, 43 mg-52 mg, 43 mg-54 mg, 43 mg-56 mg, 43 mg-58 mg, 42 mg-60 mg, 45 mg-48 mg, 45 mg-50 mg, 45 mg-52 mg, 45 mg-54 mg, 45 mg-56 mg, 45 mg-58 mg, 45 mg-60 mg, 48 mg-50 mg, 48 mg-52 mg, 48 mg-54 mg, 48 mg-56 mg, 48 mg-58 mg, 48 mg-60 mg, 50 mg-52 mg, 50 mg-54 mg, 50 mg-56 mg, 50 mg-58 mg, 50 mg-60 mg, 52 mg-54 mg, 52 mg-56 mg, 52 mg-58 mg, or 52 mg-60 mg. In some embodiments, the readthrough modulator compound dose is greater than about 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, or about 200 mg. In some embodiments, the readthrough modulator compound dose is about less than about 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, or about 200 mg. In some embodiments, the readthrough modulator compound dose is about 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, about 200 mg, about 225 mg, about 250 mg, about 275 mg, or about 300 mg.

In some embodiments, the additional pharmaceutical agent is administered at a dose in the range of about 1-50 mg/m² of the body surface area. In some embodiments, the additional pharmaceutical agent is administered at a dose in the range of about 1-2, 1-3, 1-4, 1-5, 1-6, 1-7, 1-8, 1-9, 1-10, 1-11, 1-12, 1-13, 1-13.75, 1-14, 1-15, 1-16, 1-17, 1-18, 1-19, 1-20, 1-22.5, 1-25, 1-27.5, 1-30, 1.5-2, 1.5-3, 1.5-4, 1.5-5, 1.5-6, 1.5-7, 1.5-8, 1.5-9, 1.5-10, 1.5-11, 1.5-12, 1.5-13, 1.5-13.75, 1.5-14, 1.5-15, 1.5-16, 1.5-17, 1.5-18, 1.5-19, 1.5-20, 1.5-22.5, 1.5-25, 1.5-27.5, 1.5-30, 2.5-2, 2.5-3, 2.5-4, 2.5-5, 2.5-6, 2.5-7, 2.5-8, 2.5-9, 2.5-10, 2.5-11, 2.5-12, 2.5-13, 2.5-13.75, 2.5-14, 2.5-15, 2.5-16, 2.5-17, 2.5-18, 2.5-19, 2.5-20, 2.5-22.5, 2.5-25, 2.5-27.5, 2.5-30, 2.5-7.5, 3-4, 3-5, 3-6, 3-7, 3-8, 3-9, 3-10, 3-11, 3-12, 3-13, 3-13.75, 3-14, 3-15, 3-16, 3-17, 3-18, 3-19, 3-20, 3-22.5, 3-25, 3-27.5, 3-30, 3.5-6.5, 3.5-13.75, 3.5-15, 2.5-17.5, 4-5, 4-6, 4-7, 4-8, 4-9, 4-10, 4-11, 4-12, 4-13, 4-13.75, 4-14, 4-15, 4-16, 4-17, 4-18, 4-19, 4-20, 4-22.5, 4-25, 4-27.5, 4-30, 5-6, 5-7, 5-8, 5-9, 5-10, 5-11, 5-12, 5-13, 5-13.75, 5-14, 5-15, 5-16, 5-17, 5-18, 5-19, 5-20, 5-22.5, 5-25, 5-27.5, 5-30, 6-7, 6-8, 6-9, 6-10, 6-11, 6-12, 6-13, 6-13.75, 6-14, 6-15, 6-16, 6-17, 6-18, 6-19, 6-20, 6-22.5, 6-25, 6-27.5, 6-30, 7-8, 7-9, 7-10, 7-11, 7-12, 7-13, 7-13.75, 7-14, 7-15, 7-16, 7-17, 7-18, 7-19, 7-20, 7-22.5, 7-25, 7-27.5, 7-30, 7.5-12.5, 7.5-13.5, 7.5-15, 8-9, 8-10, 8-11, 8-12, 8-13, 8-13.75, 8-14, 8-15, 8-16, 8-17, 8-18, 8-19, 8-20, 8-22.5, 8-25, 8-27.5, 8-30, 9-10, 9-11, 9-12, 9-13, 9-13.75, 9-14, 9-15, 9-16, 9-17, 9-18, 9-19, 9-20, 9-22.5, 9-25, 9-27.5, 9-30, 10-11, 10-12, 10-13, 10-13.75, 10-14, 10-15, 10-16, 10-17, 10-18, 10-19, 10-20, 10-22.5, 10-25, 10-27.5, 10-30, 11.5-15.5, 12.5-14.5, 7.5-22.5, 8.5-32.5, 9.5-15.5, 15.5-24.5, 5-35, 17.5-22.5, 22.5-32.5, 25-35, 25.5-24.5, 27.5-32.5, 2-20, 2.5-22.5, or 9.5-21.5 mg/m², of the body surface area. In some embodiments, the additional pharmaceutical agent is administered at a dose of about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40 mg/m² of the body surface area. In some embodiments, the additional pharmaceutical agent is administered at a dose less than about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40 mg/m² of the body surface area. In some embodiments, the additional pharmaceutical agent is administered at a dose greater than about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40, 41, 42, 43, 44, 45, 46, 47, 48, 49, 50 mg/m² of the body surface area.

In some embodiments, the additional pharmaceutical agent dose is about 5 mg-300 mg, 5 mg-200 mg, 7.5 mg-200 mg, 10 mg-100 mg, 15 mg-100 mg, 20 mg-100 mg, 30 mg-100 mg, 40 mg-100 mg, 10 mg-80 mg, 15 mg-80 mg, 20 mg-80 mg, 30 mg-80 mg, 40 mg-80 mg, 10 mg-60 mg, 15 mg-60 mg, 20 mg-60 mg, 30 mg-60 mg, or about 40 mg-60 mg. In some embodiments, the additional pharmaceutical agent dose administered is about 20 mg-60 mg, 27 mg-60 mg, 20 mg-45 mg, or 27 mg-45 mg. In some embodiments, the additional pharmaceutical agent dose administered is about 5 mg-7.5 mg, 5 mg-9 mg, 5 mg-10 mg, 5 mg-12 mg, 5 mg-14 mg, 5 mg-15 mg, 5 mg-16 mg, 5 mg-18 mg, 5 mg-20 mg, 5 mg-22 mg, 5 mg-24 mg, 5 mg-26 mg, 5 mg-28 mg, 5 mg-30 mg, 5 mg-32 mg, 5 mg-34 mg, 5 mg-36 mg, 5 mg-38 mg, 5 mg-40 mg, 5 mg-42 mg, 5 mg-44 mg, 5 mg-46 mg, 5 mg-48 mg, 5 mg-50 mg, 5 mg-52 mg, 5 mg-54 mg, 5 mg-56 mg, 5 mg-58 mg, 5 mg-60 mg, 7 mg-7.7 mg, 7 mg-9 mg, 7 mg-10 mg, 7 mg-12 mg, 7 mg-14 mg, 7 mg-15 mg, 7 mg-16 mg, 7 mg-18 mg, 7 mg-20 mg, 7 mg-22 mg, 7 mg-24 mg, 7 mg-26 mg, 7 mg-28 mg, 7 mg-30 mg, 7 mg-32 mg, 7 mg-34 mg, 7 mg-36 mg, 7 mg-38 mg, 7 mg-40 mg, 7 mg-42 mg, 7 mg-44 mg, 7 mg-46 mg, 7 mg-48 mg, 7 mg-50 mg, 7 mg-52 mg, 7 mg-54 mg, 7 mg-56 mg, 7 mg-58 mg, 7 mg-60 mg, 9 mg-10 mg, 9 mg-12 mg, 9 mg-14 mg, 9 mg-15 mg, 9 mg-16 mg, 9 mg-18 mg, 9 mg-20 mg, 9 mg-22 mg, 9 mg-24 mg, 9 mg-26 mg, 9 mg-28 mg, 9 mg-30 mg, 9 mg-32 mg, 9 mg-34 mg, 9 mg-36 mg, 9 mg-38 mg, 9 mg-40 mg, 9 mg-42 mg, 9 mg-44 mg, 9 mg-46 mg, 9 mg-48 mg, 9 mg-50 mg, 9 mg-52 mg, 9 mg-54 mg, 9 mg-56 mg, 9 mg-58 mg, 9 mg-60 mg, 10 mg-12 mg, 10 mg-14 mg, 10 mg-15 mg, 10 mg-16 mg, 10 mg-18 mg, 10 mg-20 mg, 10 mg-22 mg, 10 mg-24 mg, 10 mg-26 mg, 10 mg-28 mg, 10 mg-30 mg, 10 mg-32 mg, 10 mg-34 mg, 10 mg-36 mg, 10 mg-38 mg, 10 mg-40 mg, 10 mg-42 mg, 10 mg-44 mg, 10 mg-46 mg, 10 mg-48 mg, 10 mg-50 mg, 10 mg-52 mg, 10 mg-54 mg, 10 mg-56 mg, 10 mg-58 mg, 10 mg-60 mg, 12 mg-14 mg, 12 mg-15 mg, 12 mg-16 mg, 12 mg-18 mg, 12 mg-20 mg, 12 mg-22 mg, 12 mg-24 mg, 12 mg-26 mg, 12 mg-28 mg, 12 mg-30 mg, 12 mg-32 mg, 12 mg-34 mg, 12 mg-36 mg, 12 mg-38 mg, 12 mg-40 mg, 12 mg-42 mg, 12 mg-44 mg, 12 mg-46 mg, 12 mg-48 mg, 12 mg-50 mg, 12 mg-52 mg, 12 mg-54 mg, 12 mg-56 mg, 12 mg-58 mg, 12 mg-60 mg, 15 mg-16 mg, 15 mg-18 mg, 15 mg-20 mg, 15 mg-22 mg, 15 mg-24 mg, 15 mg-26 mg, 15 mg-28 mg, 15 mg-30 mg, 15 mg-32 mg, 15 mg-34 mg, 15 mg-36 mg, 15 mg-38 mg, 15 mg-40 mg, 15 mg-42 mg, 15 mg-44 mg, 15 mg-46 mg, 15 mg-48 mg, 15 mg-50 mg, 15 mg-52 mg, 15 mg-54 mg, mg-56 mg, 15 mg-58 mg, 15 mg-60 mg, 17 mg-18 mg, 17 mg-20 mg, 17 mg-22 mg, 17 mg-24 mg, 17 mg-26 mg, 17 mg-28 mg, 17 mg-30 mg, 17 mg-32 mg, 17 mg-34 mg, 17 mg-36 mg, 17 mg-38 mg, 17 mg-40 mg, 17 mg-42 mg, 17 mg-44 mg, 17 mg-46 mg, 17 mg-48 mg, 17 mg-50 mg, 17 mg-52 mg, 17 mg-54 mg, 17 mg-56 mg, 17 mg-58 mg, 17 mg-60 mg, 20 mg-22 mg, 20 mg-24 mg, 20 mg-26 mg, 20 mg-28 mg, 20 mg-30 mg, 20 mg-32 mg, 20 mg-34 mg, 20 mg-36 mg, 20 mg-38 mg, mg-40 mg, 20 mg-42 mg, 20 mg-44 mg, 20 mg-46 mg, 20 mg-48 mg, 20 mg-50 mg, 20 mg-52 mg, mg-54 mg, 20 mg-56 mg, 20 mg-58 mg, 20 mg-60 mg, 22 mg-24 mg, 22 mg-26 mg, 22 mg-28 mg, 22 mg-30 mg, 22 mg-32 mg, 22 mg-34 mg, 22 mg-36 mg, 22 mg-38 mg, 22 mg-40 mg, 22 mg-42 mg, 22 mg-44 mg, 22 mg-46 mg, 22 mg-48 mg, 22 mg-50 mg, 22 mg-52 mg, 22 mg-54 mg, 22 mg-56 mg, 22 mg-58 mg, 22 mg-60 mg, 25 mg-26 mg, 25 mg-28 mg, 25 mg-30 mg, 25 mg-32 mg, mg-34 mg, 25 mg-36 mg, 25 mg-38 mg, 25 mg-40 mg, 25 mg-42 mg, 25 mg-44 mg, 25 mg-46 mg, mg-48 mg, 25 mg-50 mg, 25 mg-52 mg, 25 mg-54 mg, 25 mg-56 mg, 25 mg-58 mg, 25 mg-60 mg, 27 mg-28 mg, 27 mg-30 mg, 27 mg-32 mg, 27 mg-34 mg, 27 mg-36 mg, 27 mg-38 mg, 27 mg-40 mg, 27 mg-42 mg, 27 mg-44 mg, 27 mg-46 mg, 27 mg-48 mg, 27 mg-50 mg, 27 mg-52 mg, 27 mg-54 mg, 27 mg-56 mg, 27 mg-58 mg, 27 mg-60 mg, 30 mg-32 mg, 30 mg-34 mg, 30 mg-36 mg, 30 mg-38 mg, mg-40 mg, 30 mg-42 mg, 30 mg-44 mg, 30 mg-46 mg, 30 mg-48 mg, 30 mg-50 mg, 30 mg-52 mg, mg-54 mg, 30 mg-56 mg, 30 mg-58 mg, 30 mg-60 mg, 33 mg-34 mg, 33 mg-36 mg, 33 mg-38 mg, 33 mg-40 mg, 33 mg-42 mg, 33 mg-44 mg, 33 mg-46 mg, 33 mg-48 mg, 33 mg-50 mg, 33 mg-52 mg, 33 mg-54 mg, 33 mg-56 mg, 33 mg-58 mg, 33 mg-60 mg, 36 mg-38 mg, 36 mg-40 mg, 36 mg-42 mg, 36 mg-44 mg, 36 mg-46 mg, 36 mg-48 mg, 36 mg-50 mg, 36 mg-52 mg, 36 mg-54 mg, 36 mg-56 mg, 36 mg-58 mg, 36 mg-60 mg, 40 mg-42 mg, 40 mg-44 mg, 40 mg-46 mg, 40 mg-48 mg, 40 mg-50 mg, 40 mg-52 mg, 40 mg-54 mg, 40 mg-56 mg, 40 mg-58 mg, 40 mg-60 mg, 43 mg-46 mg, 43 mg-48 mg, 43 mg-50 mg, 43 mg-52 mg, 43 mg-54 mg, 43 mg-56 mg, 43 mg-58 mg, 42 mg-60 mg, 45 mg-48 mg, 45 mg-50 mg, 45 mg-52 mg, 45 mg-54 mg, 45 mg-56 mg, 45 mg-58 mg, 45 mg-60 mg, 48 mg-50 mg, 48 mg-52 mg, 48 mg-54 mg, 48 mg-56 mg, 48 mg-58 mg, 48 mg-60 mg, 50 mg-52 mg, 50 mg-54 mg, 50 mg-56 mg, 50 mg-58 mg, 50 mg-60 mg, 52 mg-54 mg, 52 mg-56 mg, 52 mg-58 mg, or 52 mg-60 mg. In some embodiments, the additional pharmaceutical agent dose is greater than about 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, or about 200 mg. In some embodiments, the additional pharmaceutical agent dose is about less than about 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, or about 200 mg. In some embodiments, the additional pharmaceutical agent dose is about 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, about 200 mg, about 225 mg, about 250 mg, about 275 mg, or about 300 mg.

In some embodiments, the additional pharmaceutical agent may be an aminoglycoside described here. In other embodiments, the additional pharmaceutical agent may be an eRF3 modulator. In some specific embodiments, the eRF3 modulator may be a cereblon E3 ligase modulator. In yet other embodiments, two or more additional pharmaceutical agents may be administered in combination with the readthrough modulators described herein. In some embodiments, the readthrough modulator may be administered in combination with two additional pharmaceutical agents, wherein the two additional pharmaceutical agents may be an aminoglycoside and an eRF3 modulator. In some specific embodiments, the eRF3 modulator may be a cereblon E3 ligase modulator.

In some embodiments, the mass ratio of readthrough modulator compound to any of the additional pharmaceutical agents is from about 10:1 to about 1:10. In some embodiments, the mass ratio of readthrough modulator compound to any of the additional pharmaceutical agents is from about 7:1 to about 1:7. In some embodiments, the mass ratio of readthrough modulator compound to any of the additional pharmaceutical agents is from about 5:1 to about 1:5. In some embodiments, the mass ratio of readthrough modulator compound to any of the additional pharmaceutical agents is from about 3:1 to about 1:3. In some embodiments, the mass ratio of readthrough modulator compound to any of the additional pharmaceutical agent is from about 2:1 to about 1:2. In some embodiments, the mass ratio of readthrough modulator compound to any of the additional pharmaceutical agents is from about 10:1 to about 1:1, from about 7:1 to about 1:1, from about 5:1 to about 1:1, from about 3:1 to about 1:1, or from about 2:1 to about 1:1. In some embodiments, the mass ratio of readthrough modulator compound to any of the additional pharmaceutical agents is from about 1:1 to about 1:2, from about 1:1 to about 1:3, from about 1:1 to about 1:5, from about 1:1 to about 1:7, or from about 1:1 to about 1:10. In some embodiments, the mass ratio of readthrough modulator compound to any of the additional pharmaceuticals agents is about 10:1. 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10, or any range between two of these values.

In some embodiments, the readthrough modulator compounds presented herein may be administered simultaneously with one or more additional pharmaceutical agents. In other embodiments, the compounds of the present disclosure may be administered sequentially with one or more additional pharmaceutical agents.

In some embodiments, the readthrough modulator compounds may be administered prior to administration of the additional pharmaceutical agent. In some embodiments the readthrough modulator compounds may be administered about 15 minutes, about 30 minutes, about 45 minutes, about 1 hour, about 2 hours, about 3 hours, about 4 hours, about 6 hours, about 8 hours, about 12 hours, or about 24 hours prior to administration of an additional pharmaceutical agent provided herein. In some embodiments, the readthrough modulator compounds may be administered after administration of the additional pharmaceutical agent. In some embodiments the readthrough modulator compounds may be administered about 15 minutes, about 30 minutes, about 45 minutes, about 1 hour, about 2 hours, about 3 hours, about 4 hours, about 6 hours, about 8 hours, about 12 hours, or about 24 hours after administration of an additional pharmaceutical agent provided herein.

### Methods of Treatment

Some embodiments according to the methods and compositions of the present disclosure relate to a method of enhancing readthrough of genes containing premature codons comprising administering an effective amount of a readthrough modulator compound described herein in combination with one or more additional pharmaceutical agents (e.g., an aminoglycoside) to a subject in need thereof.

Disorders and diseases related to premature termination codons include, but are not limited to, chronic fibrosis, muscular dystrophy, retinitis pigmentosa, hemophilia A, hemophilia B, Hurler's syndrome, aniridia, ataxia-telangiectasia, tuberous sclerosis, Usher syndrome, polycystic kidney disease, CNS disease, amyloidosis, cancer, Parkinson's disease, mucopolysaccharidosis type 1, muscular mucopolysaccharidosis type 3, spinal muscular atrophysacidosis, neurofibromatosis 1 and 2, Marfan syndrome, dwarfism, hyperthyroidism, hypothyroidism, Rett syndrome, cystic fibrosis, and spinal muscular atrophy. In some specific embodiments described herein, the disease or disorder may be cystic fibrosis. In some embodiments, the disease or disorder may be cancer. Exemplary cancers include, but are not limited to, Ewing's sarcoma, heme malignancies, myeloma, glioblastoma, acute myeloid leukemia (AML), pancreatic cancer, glioma (e.g., high grade glioma (HGG) or diffuse glioma), glioblastoma, non-small cell lung cancer, diffuse large B cell lymphoma (DLBCL), breast cancer, head and neck cancer, melanoma, non-small cell lung cancer, ovarian cancer, prostate cancer, and uterine cancer. The cancer can be a solid tumor (e.g., glioma, breast tumor, lung tumor) or a hematological malignancy (e.g., AML, DLBCL, myeloma, etc.)

In some embodiments, the disease or disorder associated with premature termination codon is cystic fibrosis. Mutations in the gene that produces the 1,480 amino acid cystic fibrosis transmembrane conductance regulator (CTFR) protein can disrupt the normal production or functioning of the CFTR protein found in the cells of the lungs and other parts of the body. In some embodiments, the compounds described herein may be used to increase ribosomal readthrough of mRNA transcripts carrying a premature stop codon mutation of the cystic fibrosis CFTR channel. In some embodiments, the mutation can be one or more of G542X, R553X, R1162X, S1255X, W1282X, W1316X. In some embodiments, the mutation is G542X. In other embodiments, the mutation is R1162X.

In some embodiments, the compounds described herein, either alone or in combination with one or more additional pharmaceutical agents, or compositions comprising the same, may result in an improvement of readthrough of a premature stop codon. In some embodiments, administration of the readthrough modulator compounds described herein may result in at least 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200% or greater readthrough of a premature stop codon as compared to readthrough without administration of the readthrough modulator compounds described herein. In some embodiments, the compounds described herein may be administered in combination with an aminoglycoside. In some embodiments, administration of the readthrough modulator compounds described herein in combination with an aminoglycoside may result in at least 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, or greater readthrough of a premature stop codon as compared to administration of the aminoglycoside alone. In some embodiments, the combination of the readthrough modulator compounds described herein may be administered in combination with a compound that reduces eRF3 protein levels in a subject. In some embodiments, the combination of readthrough modulator compound described herein, aminoglycoside, and compound that reduces eRF3 protein levels in a subject may result in at least 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, or greater readthrough of a premature stop codon as compared to administration of the aminoglycoside alone. In some embodiments, the compound that reduces eRF3 protein levels in a subject is a cereblon E3 ligase modulator described herein.

### Methods of Preparation

The compounds disclosed herein may be synthesized by methods described below, or by modification of these methods. Ways of modifying the methodology include, among others, temperature, solvent, reagents etc., known to those skilled in the art. In general, during any of the processes for preparation of the compounds disclosed herein, it may be necessary and/or desirable to protect sensitive or reactive groups on any of the molecules concerned. This may be achieved by means of conventional protecting groups, such as those described in Protective Groups in Organic Chemistry (ed. J.F.W. McOmie, Plenum Press, 1973); and P.G.M. Green, T.W. Wutts, Protecting Groups in Organic Synthesis (3rd ed.) Wiley, New York (1999), which are both hereby incorporated herein by reference in their entirety. The protecting groups may be removed at a convenient subsequent stage using methods known from the art. Synthetic chemistry transformations useful in synthesizing applicable compounds are known in the art and include e.g. those described in R. Larock, Comprehensive Organic Transformations, VCH Publishers, 1989, or L. Paquette, ed., Encyclopedia of Reagents for Organic Synthesis, John Wiley and Sons, 1995, which are both hereby incorporated herein by reference in their entirety. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

### Synthesis of Compounds of Formula (I)

The following example schemes are provided for the guidance of the reader, and collectively represent an example method for making the compounds encompassed herein. Furthermore, other methods for preparing compounds described herein will be readily apparent to the person of ordinary skill in the art in light of the following reaction schemes and examples. Unless otherwise indicated, all variables are as defined above. Characterization of the final product via 1H-NMR and LCMS spectra summarized in Table A bellow.

Compounds of Formula (I) were synthesized via two distinct strategies depicted in Scheme 1, where X is halogen, complemented by added and/or inserted steps of functional group and protecting group manipulation.

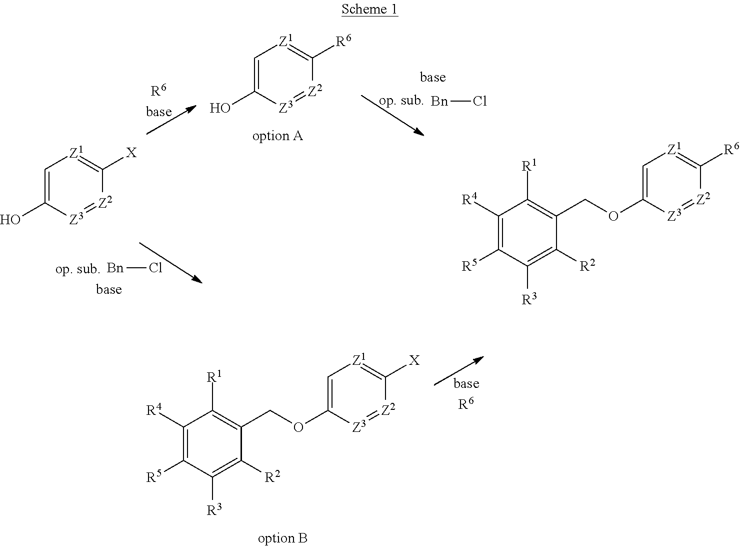

### EXAMPLES

### Examples 1 and 2

Synthesis of [1-[5-[(2,6-dichlorophenyl) methoxy]pyrimidin-2-yl]imidazol-4-yl]methanol (Compound 2)

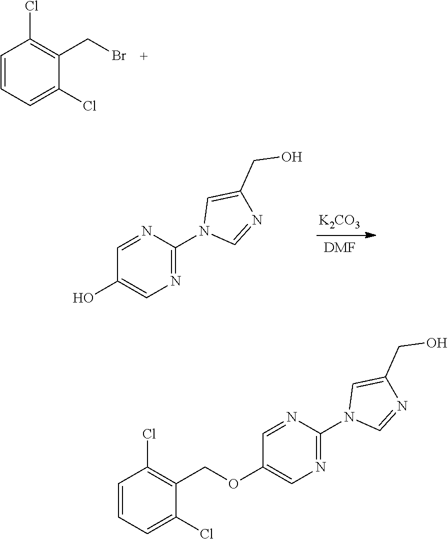

2,6-Dichlorobenzyl bromide (102.37 mg, 0.43 mmol, 1 eq), 2-[4-(hydroxymethyl) imidazole-1-yl]pyrimidin-5-ol (82 mg, 0.43 mmol, 1 eq) and potassium carbonate, (294.86 mg, 2.13 mmol, 5 eq) in dimethylformamide (DMF) (2 mL) were stirred at room temperature over the weekend under a nitrogen atmosphere. LC-MS shows the reaction had gone to completion. The reaction was diluted with ethyl acetate then washed with water (x2) followed by brine. The organic extract was dried (MgSO₄) then the remaining solvent was removed in vacuo. The resultant residue was triturated in diethyl ether and then collected by filtration. The resultant solid was dried overnight under vacuum at 40° C. to give [1-[5-[(2,6-dichlorophenyl) methoxy]pyrimidin-2-yl]imidazol-4-yl]methanol (89.71 mg, 0.26 mmol, 59.87%) as a white solid, Purity: >95%).

### Example 2

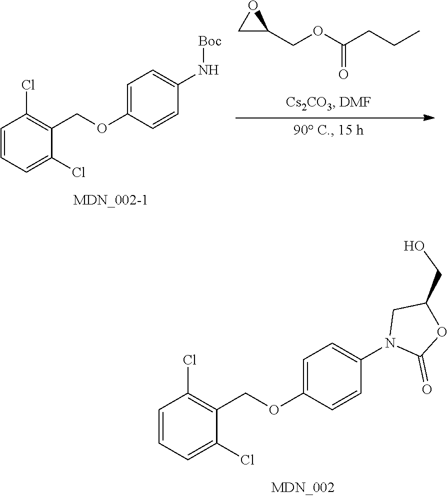

A mixture of MDN_002-1 (200 mg, 0.540 mmol), (R)-oxiran-2-ylmethyl butyrate (238 mg, 1.65 mmol) and Cs₂CO₃ (536 mg, 1.65 mmol) in DMF (4 mL) was stirred at 90oC for 15 h. After cooling, the reaction mixture was diluted with EtOAc (40 mL), washed with water (30 mL×2) and brine (30 mL). The organic layer was dried over Na2SO4, filtered and concentrated in vacuo. The residue was purified by Prep-TLC (petroleum ether (PE): EtOAc=1:1) to get yellow oil, which was purified by Prep-HPLC to afford a white solid (39.3 mg, 19.7% yield).

### Example 3

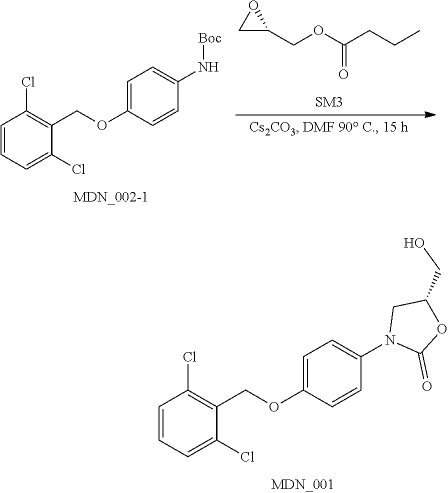

A mixture of MDN_002-1 (300 mg, 0.810 mmol), (S)-oxiran-2-ylmethyl butyrate (353 mg, 2.45 mmol) and Cs₂CO₃ (797 mg, 2.45 mmol) in DMF (6 mL) was stirred at 90oC for 15 h. After cooling, the reaction mixture was diluted with EtOAc (40 mL), washed with water (30 mL×2) and brine (30 mL). The organic layer was dried over Na2SO4, filtered and concentrated in vacuo. The residue was purified by Prep-TLC (PE: EtOAc=1:1) to get yellow oil, which was purified by Prep-HPLC to afford MDN_001 as a white solid (61.7 mg, 20.6% yield).

### Example 4

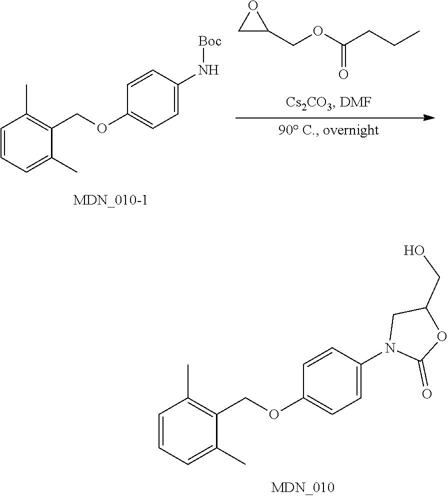

A mixture of MDN_010-1 (250 mg, 0.764 mmol), oxiran-2-ylmethyl butyrate (331 mg, 2.29 mmol) and Cs₂CO₃ (747 mg, 2.29 mmol) in DMF (5 mL) was stirred at 90oC overnight. The reaction mixture was diluted with water (30 mL), extracted with EA (30 mL×2). The organic layer was washed with brine (30 mL), dried over Na2SO4, filtered and concentrated. The residue was purified by silica gel column (PE:EA=1:1) and Prep-HPLC trifluoroacetic acid (TFA) to give MDN_010 as a white solid (42.0 mg, 17% yield).

### Example 5

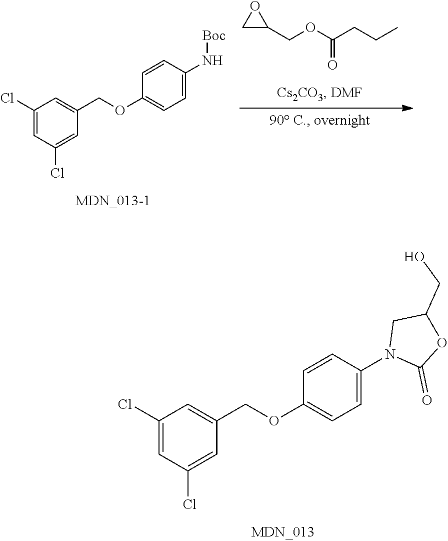

A mixture of MDN_013-1 (250 mg, 0.679 mmol), oxiran-2-ylmethyl butyrate (294 mg, 2.04 mmol) and Cs₂CO₃ (664 mg, 2.04 mmol) in DMF (5 mL) was stirred at 90oC overnight. The reaction mixture was diluted with water (30 mL), extracted with EA (30 mL x 2. The organic layer was washed with brine (30 mL), dried over Na2SO4, filtered and concentrated, purified by silica gel column (PE:EA=1:2) and Prep-HPLC (TFA) to give pure MDN_013 as a white solid (78.0 mg, 31% yield).

### Example 6

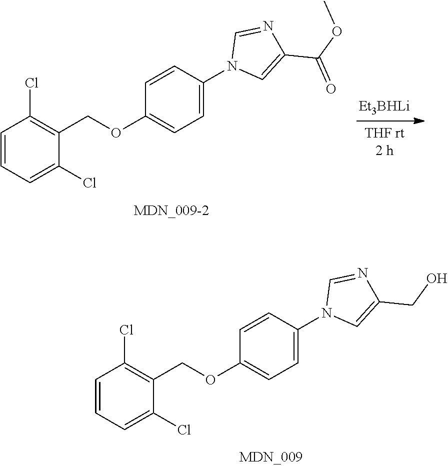 [SMILES: COC(=O)c1cn(-c2ccc(OCc3c(Cl)cccc3Cl)cc2)cn1 ; "Et3BHLi" ; OCc1cn(-c2ccc(OCc3c(Cl)cccc3Cl)cc2)cn1]

To a solution of MDN_009-2 (40 mg, 0.106 mmol) in THF (2 mL) was added Lithium triethylborohydride (1 M in THF, 1.06 mL, 1.06 mmol) at room temperature. The mixture was stirred at room temperature for 2 h. The mixture was quenched with NH4Cl aq, extracted with EA, purified by reversed phase column (H₂O: MeCN) to give the title compound MDN_009 as an off-white solid (20 mg, 54% yield).

### Example 7

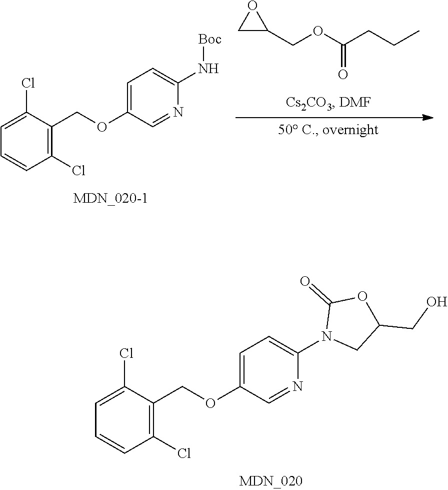

To a solution of MDN_020-1 (160 mg, 0.433 mmol) in DMF (2 ml) were added oxiran-2-ylmethyl butyrate (187 mg, 1.30 mmol) and Cs₂CO₃ (424 mg, 1.30 mmol). The reaction was stirred at 50° C. overnight. The reaction mixture was filtered and purified by prep-HPLC (NH4HCO3) to afford a white solid (40.9 mg, 25.6% yield).

### Example 8

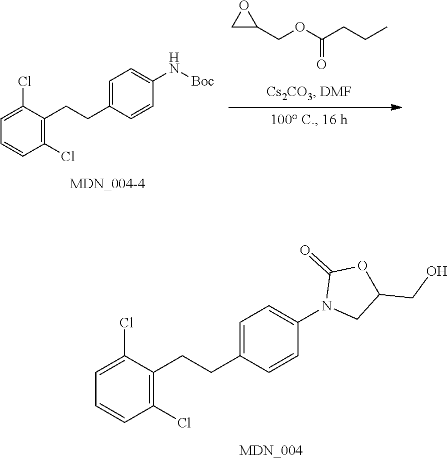

To a solution of MDN_004-4 (70 mg, 0.191 mmol) in DMF (2 mL) were added oxiran-2-ylmethyl butyrate (83 mg, 0.573 mmol) and Cs₂CO₃ (187 mg, 0.573 mmol). The reaction was stirred at 50° C. for 18 h, heated to 100° C. for 18 hours, then stirred at 100° C. in a microwave reactor for 2 h. The mixture was filtered and purified by prep-HPLC (NH4HCO3) and lyophilized to afford a white solid (13.4 mg, 19.2% yield).

### Example 9

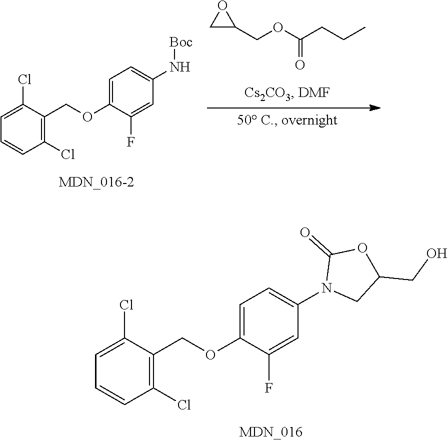

To a solution of MDN_016-2 (110 mg, 0.285 mmol) in DMF (2 ml) were added oxiran-2-ylmethyl butyrate (123 mg, 0.855 mmol) and Cs₂CO₃ (278 mg, 0.855 mmol). The reaction was stirred at 50° C. overnight. The reaction mixture was diluted with H₂O (30 mL) and extracted with EtOAc (30 mL×3). The organic layer was dried over Na2SO4, filtered and concentrated. The residue was purified by prep-HPLC (NH4HCO3) and lyophilized to afford a white solid (23.2 mg, 21.0% yield).

### Example 10

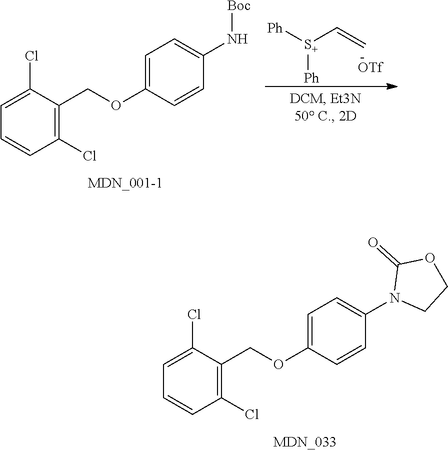

To a solution of MDN_001-1 (100 mg, 0.272 mmol) in DCM (3 mL) were added diphenyl (vinyl) sulfonium trifluoromethanesulfonate (295 mg, 0.816 mmol) and Et₃N (82 mg, 0.816 mmol). The reaction was stirred at 50° C. for 2 days. The reaction mixture was diluted with H₂O (50 ml) and extracted with DCM (50 mL×2). The organic layer was dried over Na2SO4, filtered and concentrated in vacuo. The residue was purified by flash chromatography (MeOH:DCM=0%˜5%) to afford a white solid (49.4 mg, 53.7% yield).

### Example 11

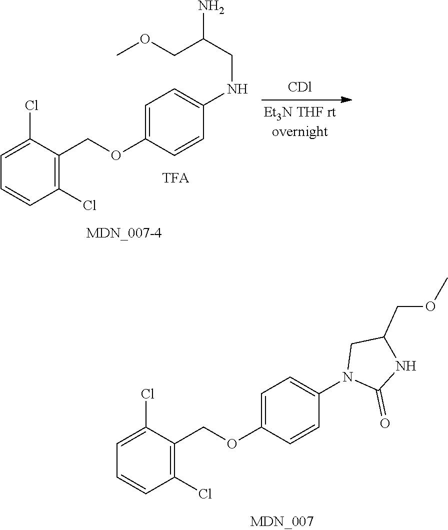 [SMILES: COCC(N)CNc1ccc(OCc2c(Cl)cccc2Cl)cc1 ; COCC1CN(c2ccc(OCc3c(Cl)cccc3Cl)cc2)C(=O)N1]

To a solution of MDN_007-4 (85 mg, 0.175 mmol) and Et₃N (53 mg, 0.525 mmol) in THF (1 mL) was added carbonyl diimidazole (CDI) (43 mg, 0.262 mmol). The mixture was stirred at room temperature overnight. The mixture was concentrated, purified by silica gel column (DCM:MeOH=40:1) to give the title compound MDN_007 as a brown solid (35 mg, 53% yield).

### Example 12

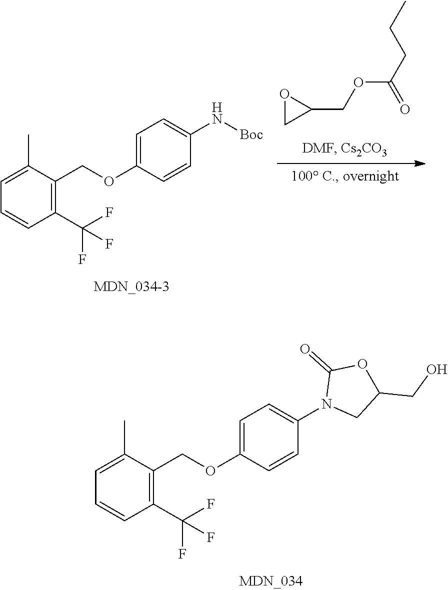

To a solution of MDN_034-3 (80 mg, 0.210 mmol) in DMF (2 mL) was added oxiran-2-ylmethyl butyrate (91 mg, 0.630 mmol) and Cs₂CO₃ (205 mg, 0.630 mmol). The reaction was stirred at 80° C. overnight. The reaction mixture was filtered, purified by prep-HPLC (NH4HCO3) and lyophilized to afford the title compound (27.7 mg, 34.6% yield).

### Example 13

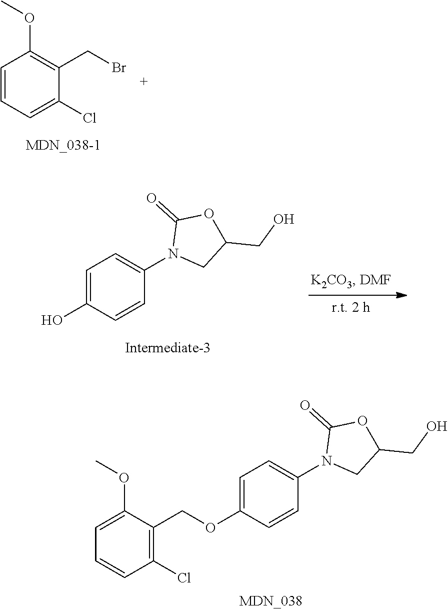 [SMILES: COc1cccc(Cl)c1CBr ; O=C1OC(CO)CN1c1ccc(O)cc1 ; COc1cccc(Cl)c1COc1ccc(N2CC(CO)OC2=O)cc1]

To a solution of MDN_038-1 (77 mg, 0.3 mmol) in DMF (2 mL) were added intermediate-3 (63 mg, 0.3 mmol) and K₂CO₃ (235 mg, 0.9 mmol). The reaction was stirred at r.t. under Argon for 16 houea. The reaction mixture was diluted with H₂O (30 mL) and extracted with EtOAc (30 mL×3). The organic layer was dried over Na2SO4, filtered and concentrated in vacuo. The residue was purified by flash chromatography (EtOAc: PE=0%˜50%) to afford a white solid (36 mg, 33% yield).

### Example 14

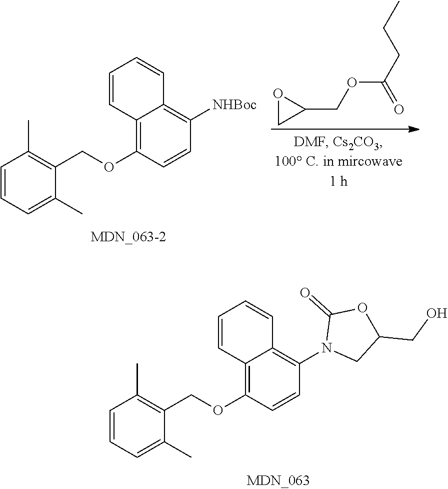

A mixture of MDN_063-2 (200 mg, 0.53 mmol), oxiran-2-ylmethyl butyrate (153 mg, 1.06 mmol) and Cs₂CO₃ (345 mg, 1.06 mmol) in DMF (2 mL) was stirred at 1000° C. in microwave for 1 hour. The mixture was filtered, purified by pre-HPLC and SFC to give the title compound MDN_063 as a white solid (21 mg, 10% yield).

### Example 15

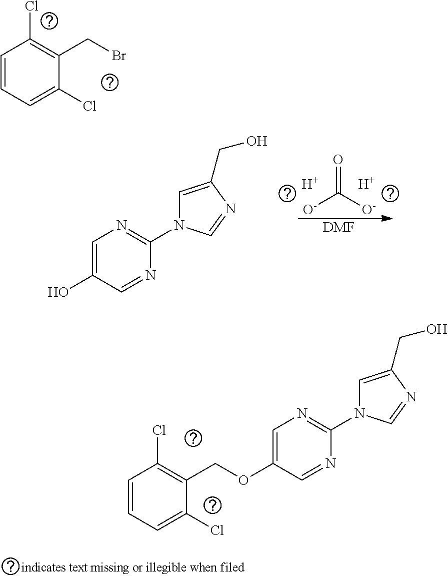

2,6-Dichlorobenzyl bromide, VERC101398:2 (102.37 mg, 0.43 mmol, 1 eq), 2-[4-(hydroxymethyl) imidazol-1-yl]pyrimidin-5-ol, 3527-RJP-160-001 (82 mg, 0.43 mmol, 1 eq) and K₂CO₃ (294.86 mg, 2.13 mmol, 5 eq) in DMF (2 mL) were stirred at rt over the weekend (a suspension formed during this time). The reaction was diluted with EtOAc then washed with water followed by brine. The organic extract was dried (MgSO₄) then the solvent removed in vacuo. The solid was dissolved DMSO for purification by prep but the sample would not fully go into solution. EtOAc was added then the solution washed with water followed by brine. The organic extract was dried (MgSO₄) then the solvent removed in vacuo. The resultant residue was triturated in diethyl ether then collected by filtration. The resultant solid was dried overnight under vacuum at 40° C. Gave (1-{5-[(2,6-dichlorophenyl) methoxy]pyrimidin-2-yl}imidazol-4-yl) methanol (89.71 mg, 0.26 mmol, 59.87%) as a white solid (Purity: >95%).

### Example 16

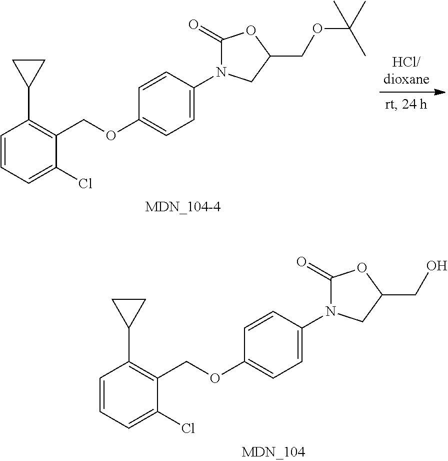 [SMILES: CC(C)(C)OCC1CN(c2ccc(OCc3c(Cl)cccc3C3CC3)cc2)C(=O)O1 ; O=C1OC(CO)CN1c1ccc(OCc2c(Cl)cccc2C2CC2)cc1]

A mixture of MDN_104-4 (80 mg, 0.180 mmol) in HCl/dioxane (2 mL) was stirred at room temperature for 24 hours. The reaction mixture was concentrated. The residue was purified by prep-HPLC (TFA) to obtain MDN_104 (12 mg, 17.8% yield, as a white solid).

### Example 17

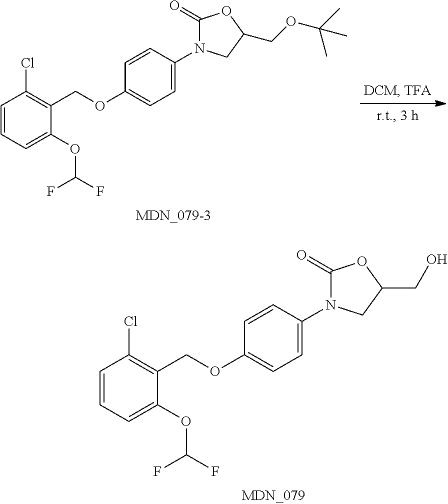 [SMILES: CC(C)(C)OCC1CN(c2ccc(OCc3c(Cl)cccc3OC(F)F)cc2)C(=O)O1 ; O=C1OC(CO)CN1c1ccc(OCc2c(Cl)cccc2OC(F)F)cc1]

To a solution of MDN_079-3 (50 mg, 0.110 mmol) in DCM (2 mL) was added TFA (1 mL). The reaction was stirred at room temperature for 3 h. The reaction mixture was concentrated in vacuo, diluted with H₂O (30 mL) and neutralized with aqueous NaHCO₃ to pH 8. The aqueous phase was extracted with EtOAc (30 mL×3). The organic layer was dried over Na2SO4, filtered and concentrated in vacuo. The residue was purified by flash chromatography (EtOAc: PE-0%˜60%) to afford a white solid (28.8 mg, 65.5% yield).

### Example 19

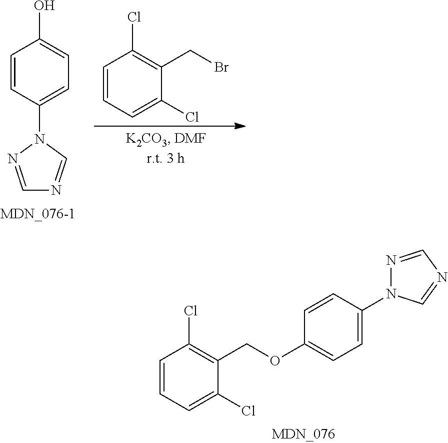

To a mixture of MDN_076-1 (70 mg, 0.434 mmol) in DMF (3 mL) were added 2-(bromomethyl)-1,3-dichlorobenzene (114 mg, 0.477 mmol) and K₂CO₃ (180 mg, 1.30 mmol). The reaction was stirred at r.t. for 3 h. LCMS indicated most of the starting material was consumed and the desired product was detected. The reaction mixture was diluted with H₂O (30 mL) and extracted with EtOAc (30 mL×3). The combined organic layer was washed with brine (20 mL×2), dried over anhydrous Na2SO4 and concentrated to give the crude product (300 mg) as a solid. The crude product was purified by flash chromatography (silica gel, 40 g, CH₃CN/H2O=0%˜100%) to give the target product MDN_076 (20 mg, 14.4% yield).

### Example 20

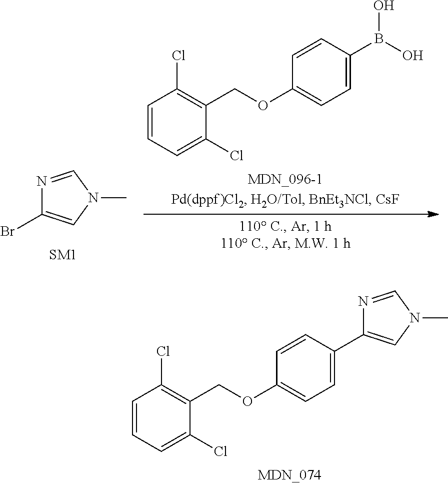

A micro tube was charged with a mixture of SM1 (50 mg, 0.310 mmol), MDN_096-1 (184 mg, 0.620 mmol), CsF (142 mg, 0.935 mmol), Pd (dppf) Cl₂ (11 mg, 0.015 mmol), BnEt₃NCI (4 mg, 0.018 mmol) in Tol (1 mL) and H₂O (1 mL). Ar was bubbled into the reaction mixture. The reaction was sealed and stirred at 110° C. for 1 h, then stirred in a microwave reactor 110° C. for 1 h. The reaction mixture was filtered, purified by prep-HPLC (NH4HCO3) and lyophilized to afford the title compound (64.3 mg, 62.2% yield).

### Example 21

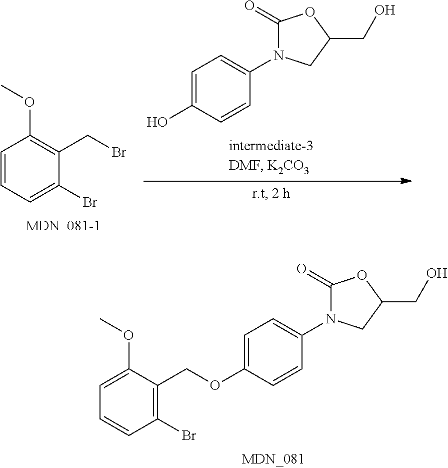 [SMILES: COc1cccc(Br)c1CBr ; O=C1OC(CO)CN1c1ccc(O)cc1 ; COc1cccc(Br)c1COc1ccc(N2CC(CO)OC2=O)cc1]

A mixture of MDN_081-1 (67 mg, 0.239 mmol), intermediate-3 (50.0 mg, 0.239 mmol) and K₂CO₃ (66.0 mg, 0.478 mmol) in DMF (2 mL) was stirred at r.t. under Ar for 16 h. The reaction mixture was diluted with H₂O (30 mL) and extracted with EtOAc (30 mL×3). The organic layer was dried over Na2SO4, filtered and concentrated in vacuo. The residue was purified by flash chromatography (EtOAc: PE=0%˜50%) to afford a white solid (50 mg, 14.0% yield).

### Example 22

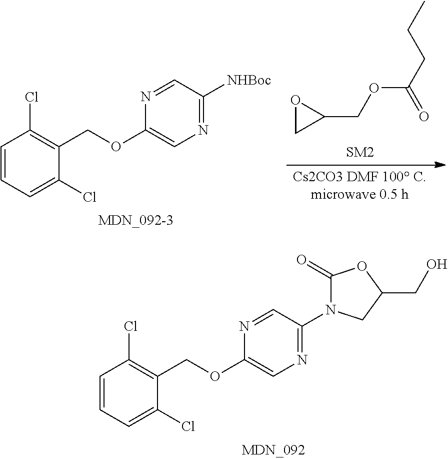

A mixture of MDN_092-3 (30 mg, 0.08 mmol), SM2 (23 mg, 0.16 mmol), and Cs₂CO₃ (52 mg, 0.16 mmol) in DMF (1 mL) was stirred at 100° C. in microwave for 0.5 h. The mixture was filtered, purified by pre-HPLC to give the title compound MDN_092 as a white solid (10 mg, 33% yield).

### Example 23

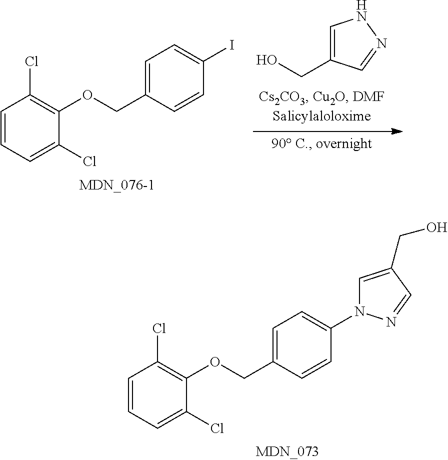

To a mixture of MDN_076-1 (100 mg, 0.264 mmol) in DMF (2 mL) were added (1H-pyrazol-4-yl) methanol (25.0 mg, 0.264 mmol), Cu₂O (2 mg, 0.016 mmol), Cs₂CO₃ (258 mg, 0.792 mmol) and Salicylaldoxime (7 mg, 0.053 mmol). The reaction was stirred at 90° C. under Ar for 16 h. The reaction mixture was diluted with H₂O (30 mL) and extracted with EtOAc (50 mL×3). The organic layer was dried over Na2SO4, filtered and concentrated in vacuo. The residue was purified by flash chromatography (EA:PE=0%˜30%) to afford a white solid (39.0 mg, 42.3% yield).

### Example 24

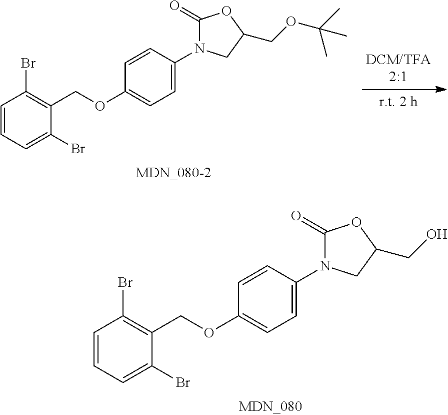 [SMILES: CC(C)(C)OCC1CN(c2ccc(OCc3c(Br)cccc3Br)cc2)C(=O)O1 ; O=C1OC(CO)CN1c1ccc(OCc2c(Br)cccc2Br)cc1]

To a mixture of MDN_080-2 (70 mg, 0.136 mmol) in DCM/TFA 2:1 (4 mL/2 mL) was stirred at r.t. for 2 h. LCMS indicated most of the starting material was consumed and the desired product was detected. The reaction mixture was diluted with H₂O (30 mL) and extracted with DCM (30 mL×3). The combined organic layer was adjusted to pH=7-8 and then washed with brine (20 mL×2), dried over anhydrous Na2SO4 and concentrated to give the crude product (130 mg) as a solid. The crude product was purified by flash chromatography (silica gel, 20 g, CH₃CN/H2O=0%˜100%) to give the target product MDN_080 (10 mg, 16.0% yield).

### Example 25

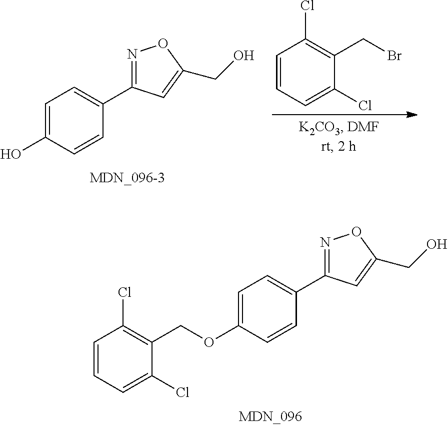

A mixture of MDN_096-3 (100 mg, 0.520 mmol), 2-(bromomethyl)-1,3-dichlorobenzene (137 mg, 0.570 mmol) and K₂CO₃ (144 mg, 1.04 mmol) in DMF (2 mL) was stirred at rt for 16 h. The mixture was filtered and purified by prep-HPLC (TFA) to obtain MDN_096 (29.1 mg, 15.9% yield) as a white solid).

### Example 26

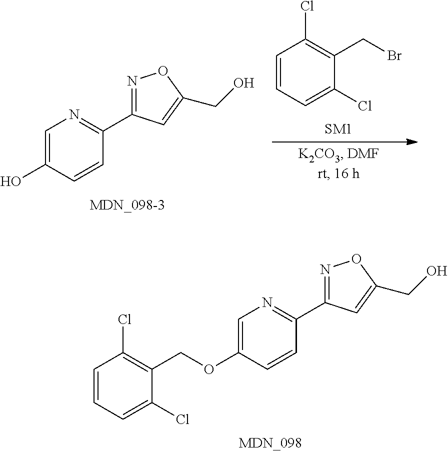

A mixture of MDN_098-3 (100 mg, 0.520 mmol), K₂CO₃ (144 mg, 1.04 mmol) and 2-(bromomethyl)-1,3-dichlorobenzene (137 mg, 0.570 mmol) in DMF (2 mL) was stirred at rt for 16 h. The mixture was filtered and purified by prep-HPLC (TFA) to obtain MDN_098 as a white solid (11.5 mg, 6.3% yield).

### Example 27

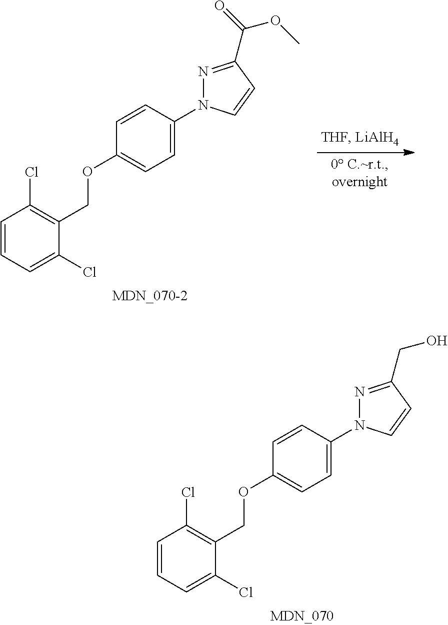 [SMILES: COC(=O)c1ccn(-c2ccc(OCc3c(Cl)cccc3Cl)cc2)n1 ; OCc1ccn(-c2ccc(OCc3c(Cl)cccc3Cl)cc2)n1]

To a solution of MDN_070-2 (145 mg, 0.384 mmol) in anhydrous THF (4 mL), was added LiAlH₄ (29 mg, 0.769 mmol) at 0° C. The reaction was stirred at r.t. under Ar overnight. The reaction mixture was diluted with H₂O (50 mL) and extracted with EtOAc (50 mL×3). The organic layer was dried over Na2SO4, filtered and concentrated in vacuo. The residue was purified by flash chromatography (EtOAc: PE=0%˜50%) to afford a white solid (25.59 mg, 19.06% yield).

### Example 28

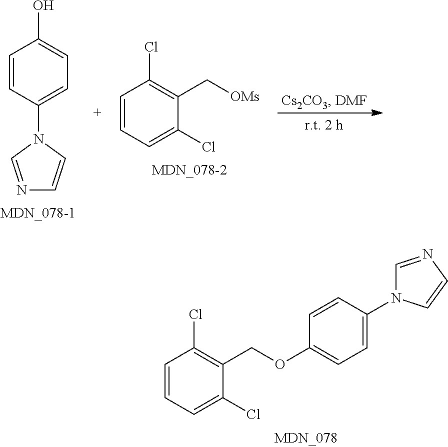

A mixture of MDN_078-1 (130 mg, 0.812 mmol), MDN_078-2 (311 mg, 1.13 mmol) and Cs₂CO₃ (793 mg, 2.44 mmol) in DMF (5 mL) was stirred at r.t. for 2 hours. The reaction mixture was diluted with H₂O (30 mL) and extracted with EtOAc (30 mL×3). The combined organic layer was washed with brine (20 mL×2), dried over anhydrous Na2SO4 and concentrated to give the crude product (278 mg) as a solid. The crude product was purified by flash chromatography (silica gel, 40 g, CH₃CN/H₂O 0%˜100%) to give the target product MDN_078 (60 mg, 23.2% yield).

### Example 29

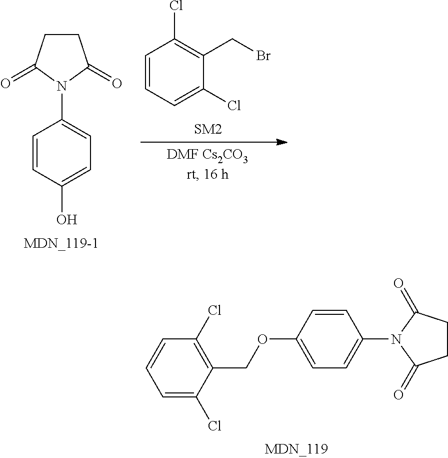

A mixture of MDN_119-1 (100 mg, 0.52 mmol), SM2 (125 mg, 0.52 mmol) and Cs₂CO₃ (341 mg, 1.1 mmol) in DMF (2 mL) was stirred at rt overnight. The mixture was quenched with water, extracted with DCM, dried over Na2SO4, purified by silica gel column to give the title compound MDN_119 as a white solid (150 mg, 82% yield).

### Example 30

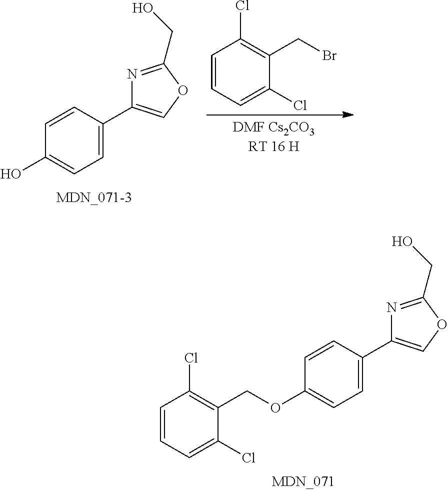

A mixture of MDN_071-3 (150 mg, 0.785 mmol), 2-(bromomethyl)-1,3-dichlorobenzene (188 mg, 0.785 mmol) and Cs₂CO₃ (512 mg, 1.57 mmol) in DMF (2 mL) was stirred at rt for 16 h. The mixture was purified by pre-HPLC to get to give the title compound MDN_071 as a white solid (24 mg, 8% yield).

### Example 31

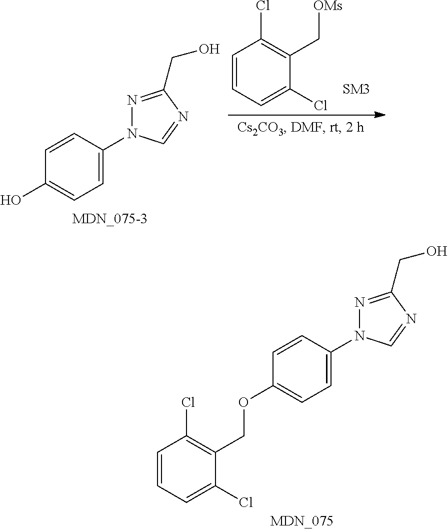

A mixture of MDN_075-3 (60 mg 0.270 mmol), 2,6-dichlorobenzyl methanesulfonate (69 mg, 0.270 mmol) and Cs₂CO₃ (263 mg, 0.810 mmol) in DMF (2 mL) was stirred at rt for 2 h. The reaction mixture was diluted with H₂O and extracted with ethyl acetate. The organic layer was dried over Na2SO4, filtered and concentrated. The residue was purified by prep-HPLC (TFA) to obtain MDN_075 (50 mg, 52.9% yield, as a white solid).

### Example 32

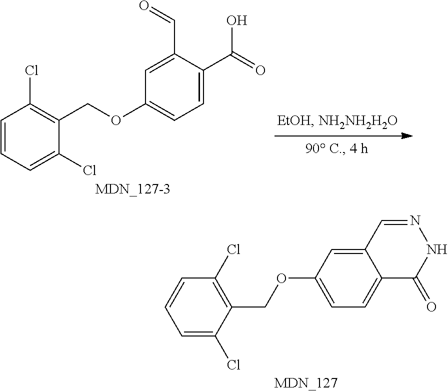 [SMILES: O=Cc1cc(OCc2c(Cl)cccc2Cl)ccc1C(=O)O ; O=c1[nH]ncc2cc(OCc3c(Cl)cccc3Cl)ccc12]

To a solution of MDN_127-3 (180 mg, 0.554 mmol) in EtOH (5 mL) was added hydrazine hydrate (80%, 5 mL). The reaction was stirred at 90° C. for 4 h. The mixture was cooled to room temperature and NaBH₃CN (354 mg, 5.64 mmol) was added. The reaction was stirred at 60° C. overnight. The reaction mixture was concentrated to remove excess volatiles. The residue was purified by prep-HPLC (NH4HCO3) to afford MDN_127 (71 mg, 39.8% yield) as a white solid.

### Example 33

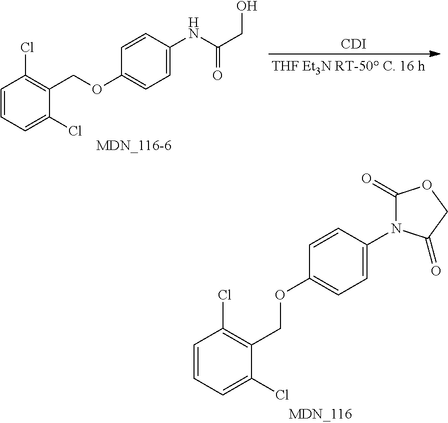 [SMILES: O=C(CO)Nc1ccc(OCc2c(Cl)cccc2Cl)cc1 ; "CDI" ; O=C1COC(=O)N1c1ccc(OCc2c(Cl)cccc2Cl)cc1]

To a mixture of MDN_116-6 (30 mg, 0.09 mmol) and Et₃N (14 mg, 0.14 mmol) in THF (0.5 mL) was added CDI (18 mg, 0.11 mmol). The mixture was stirred at rt overnight. The mixture was concentrated, purified by silica gel column to give the title compound MDN_116 as a white solid (14 mg, 44% yield).

### Example 34

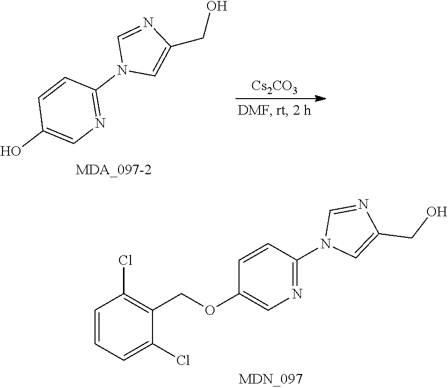

A mixture of MDN_097-1 (200 mg, 0.78 mmol), MDN_097-2 (150 mg, 0.78 mmol) and Cs₂CO₃ (760 mg, 2.34 mmol) in DMF (2 mL) was stirred at rt for 2 h. The reaction mixture was diluted with H₂O and extracted with EA. The organic layer was dried over Na2SO4, filtered and concentrated. The residue was purified by prep-HPLC (TFA) to obtain MDN_097 (25 mg, 9.2% yield, as a white solid).

### Example 35

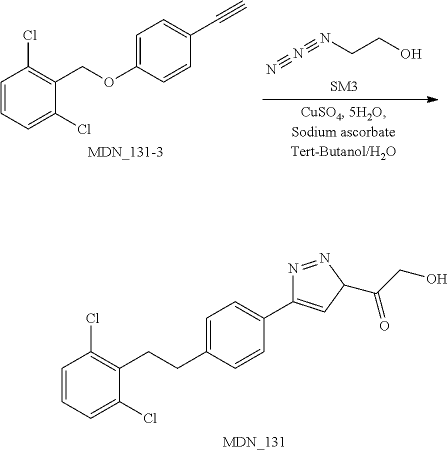

A mixture of MDN_131-3 (55 mg, 0.200 mmol), 2-azidoethan-1-ol (16.7 uL, 0.220 mmol), CuSO₄·5H₂O (5 mg, 0.020 mmol) and Sodium ascorbate (40 mg, 0.22 mmol) in Tert-Butanol/H₂O (1:1, 2 mL) was stirred at rt for 16 h. The reaction mixture was diluted with H₂O and extracted with EA. The organic layer was dried over Na2SO4, filtered and concentrated. The residue was purified by prep-HPLC (TFA) to obtain MDN_131 (3 mg, 4.1% yield) as a white solid.

### Example 36

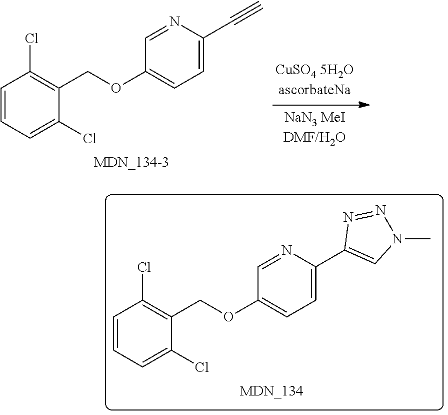 [SMILES: C#Cc1ccc(OCc2c(Cl)cccc2Cl)cn1 ; Cn1cc(-c2ccc(OCc3c(Cl)cccc3Cl)cn2)nn1]

To a mixture of NaN₃ (140 mg, 2.16 mmol), CuSO₄·5H₂O (18 mg, 0.072 mmol) and ascorbate Na (36 mg, 0.18 mmol) in DMF/H₂O (1.5 mL/1.5 mL) was added MDN_134-3 (100 mg, 0.359 mmol) and Mel (82 mg, 0.576 mmol). The mixture was stirred at 120° C. overnight. The mixture was quenched with H₂O, extracted with DCM, dried over Na2SO4, purified by silica gel column to give the title compound MDN_134 as a white solid (40 mg, 33% yield).

### Example 37

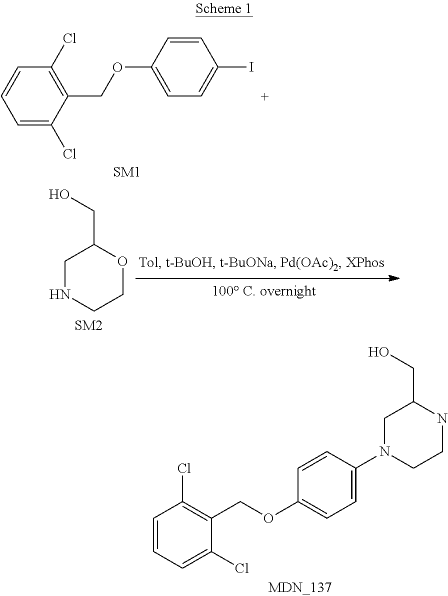

A microwave tube was charged with a mixture of 1,3-dichloro-2-((4-iodophenoxy) methyl)benzene (400 mg, 1.06 mmol), morpholin-2-ylmethanol (124 mg, 1.06 mmol), t-BuONa (152 mg, 1.58 mmol), Pd(OAc)₂ (12 mg, 0.053 mmol), dicyclohexyl [2′,4′,6′-tris (propan-2-yl) [1,1′-biphenyl]-2-yl]phosphane (XPhos) (51 mg, 0.106 mmol) and t-BuOH (4 ml) in Tol (4 ml). Argon was bubbled into the mixture for 1 minute. The reaction was sealed and stirred at 100° C. for 16 h. After cooling, the reaction mixture was concentrated to remove excess volatiles. The residue was diluted with H₂O (20 ml) and extracted with EtOAc (30 ml×3). The organic layer was dried over Na2SO4, filtered and concentrated to give the crude product, which was purified by flash chromatography on silica gel (EA/PE=0-50%) to afford the title compound as a yellow solid (66 mg, 16.9% yield).

### Example 38

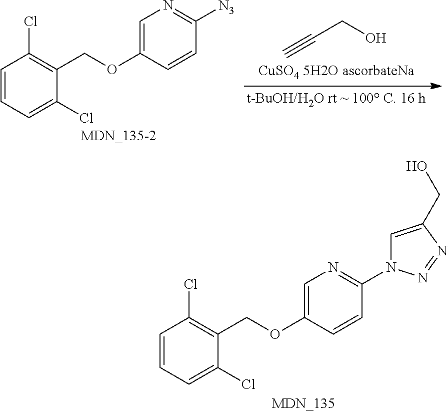

To a solution of MDN_135-2 (200 mg, 0.677 mmol) and prop-2-yn-1-ol (42 mg, 0.744 mmol) in t-BuOH/H₂O (2 ml/1 ml) was added CuSO4 5H₂O (169 mg, 0.677 mmol) and L-Ascorbic Acid Sodium Salt (67 mg, 0.338 mmol). The mixture was stirred at 100° C. for 16 h. The mixture was extracted with DCM, concentrated to give the residue, which was purified by pre-HPLC to get to give the title compound MDN_135 as a white solid (20 mg, 8% yield).

### Example 39

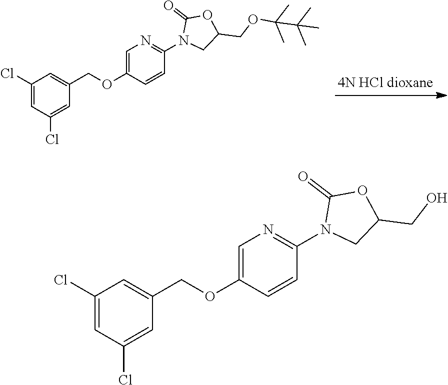 [SMILES: CC(C)(C)C(C)(C)OCC1CN(c2ccc(OCc3cc(Cl)cc(Cl)c3)cn2)C(=O)O1 ; O=C1OC(CO)CN1c1ccc(OCc2cc(Cl)cc(Cl)c2)cn1]

A solution of 3513-AF-146-001 (283 mg, 0.59 mmol, 1 eq) in HCl (4M in Dioxane) (3 mL) was stirred at rt for 2 hours. Work-up: The reaction mixture was conc in vacuum. Diluted into DMSO (3 ml) and purified by preparative HPLC automated chromatography (ISCO ACCQ HP125, Prep HPLC Column: Big Gemini-NX C18 Dimensions: 30 mm×250 mm 5 μM; Sample: 3.20 ml from tube 1) eluting with 5 to 95% ACN/0.1% FA in Water/0.1% Formic Acid (FA). Desired fractions were combined to give 3-{5-[(3,5-dichlorophenyl) methoxy]pyridin-2-yl}-5-(hydroxymethyl)-1,3-oxazolidin-2-one (103 mg, 0.28 mmol, 47.66%) white solid (Purity: >95%).

### Example 40

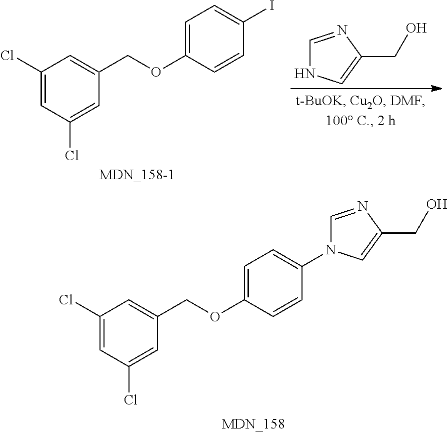 [SMILES: Clc1cc(Cl)cc(COc2ccc(I)cc2)c1 ; OCc1c[nH]cn1 ; OCc1cn(-c2ccc(OCc3cc(Cl)cc(Cl)c3)cc2)cn1]

A mixture of MDN_158-1 (190 mg, 0.500 mmol), (1H-imidazol-4-yl) methanol (50 mg, 0.500 mmol), t-BuOK (169 mg, 1.50 mmol) and Cu₂O(15 mg, 0.100 mmol) in DMF (2 mL) was stirred at 100° C. for 2 h. The reaction mixture was diluted with H₂O and extracted with ethyl acetate. The organic layer was dried over Na2SO4, filtered and concentrated. The residue was purified by prep-HPLC (TFA) to obtain MDN_158 (20 mg, 11.5% yield, as a white solid).

### Example 41

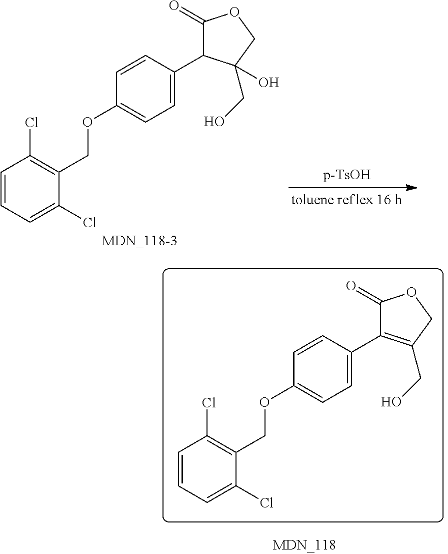 [SMILES: O=C1OCC(O)(CO)C1c1ccc(OCc2c(Cl)cccc2Cl)cc1 ; O=C1OCC(CO)=C1c1ccc(OCc2c(Cl)cccc2Cl)cc1]

A mixture of MDN_118-3 (10 mg, 0.03 mmol) and para-toluenesulfonic acid (p-TsOH) (6 mg, 0.003 mmol) in toluene (1 mL) was stirred at 120° C. overnight. The mixture was washed with water, dried over Na2SO4, purified by pre-TLC to give the title compound MDN_118 as a white solid (5 mg, 50% yield).

### Example 42

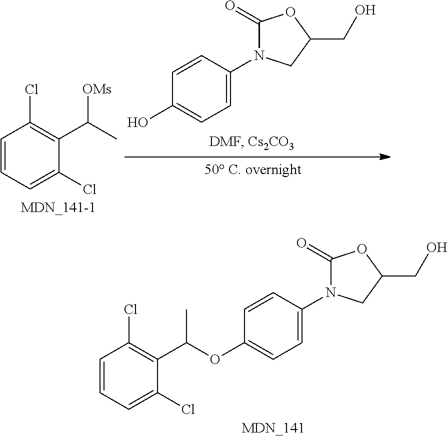

To a solution of MDN_141-1 (141 mg, 0.523 mmol) in DMF (3 mL) were added intermediate-3 (109 mg, 0.523 mmol) and Cs₂CO₃ (511 mg, 1.57 mmol). The reaction was stirred at 50° C. overnight. The reaction mixture was diluted with H₂O (40 mL) and extracted with EtOAc (40 mL×3). The organic layer was washed with brine (100 mL), dried over Na2SO4, filtered and concentrated in vacuo. The residue was purified by flash chromatography (MeOH:DCM=0%˜10%) to afford MDN_141 as a white solid (141 mg, 70.5% yield).

### Example 43

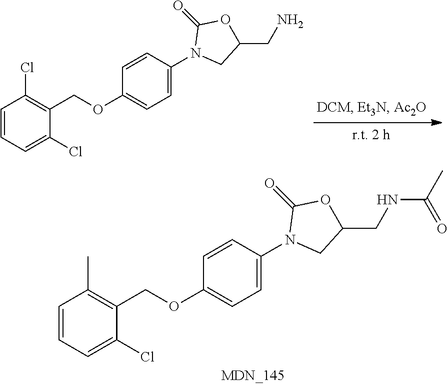 [SMILES: NCC1CN(c2ccc(OCc3c(Cl)cccc3Cl)cc2)C(=O)O1 ; CC(=O)NCC1CN(c2ccc(OCc3c(C)cccc3Cl)cc2)C(=O)O1]

To a solution of MDN_145-3 (100 mg, 0.262 mmol) and Et₃N (53 mg, 0.524 mmol) in DCM (4 mL) was added Ac2O (29 mg, 0.288 mmol). The reaction was stirred at room temperature for 2 h. The reaction mixture was diluted with H₂O (10 mL) and extracted with DCM (20 mL×3). The organic layer was dried over Na2SO4, filtered and concentrated in vacuo. The residue was purified by flash chromatography (EA:PE=0%˜50%) to afford MDN_145 as a white solid (28 mg, 25.2% yield).

### Example 44

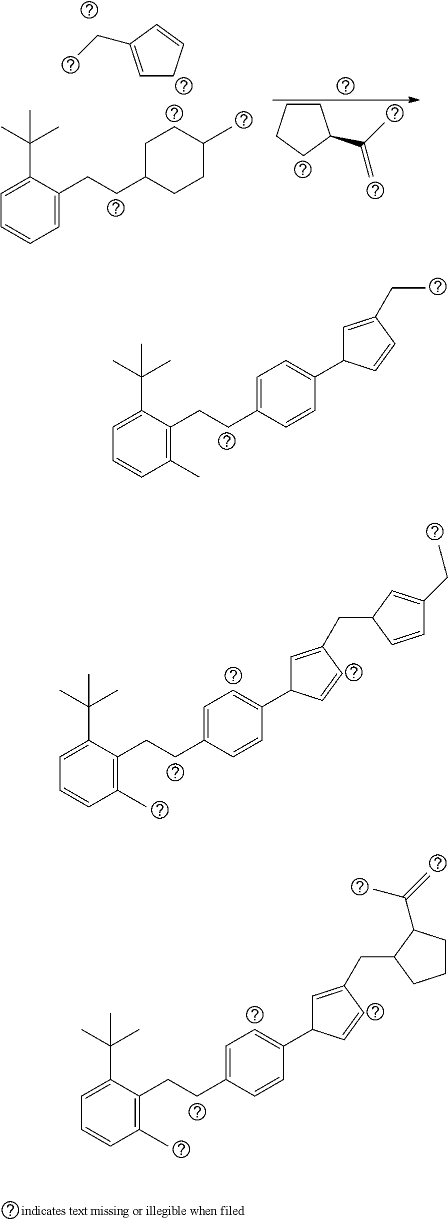

To a degassed stirred solution of 4-(HYDROXYMETHYL) IMIDAZOLE HYDROCHLORIDE (46.65 mg, 0.35 mmol, 1.2 eq), 2-bromo-5-[(2,6-dimethylphenyl) methoxy]pyridine (100 mg, 0.29 mmol, 1 eq), L-PROLINE (4.93 uL, 1.35 g/mL, 0.06 mmol, 0.2 eq) and POTASSIUM CARBONATE (119.78 mg, 0.87 mmol, 3 eq) in DMSO (3 mL) was added COPPER (I) IODIDE (5.5 mg, 0.03 mmol, 0.1 eq). The reaction was sealed and heated to 90° C. for 72 h. The reaction was cooled to rt and filtered through celite before being poured into brine (100 mL) and extracted with ethyl acetate (3×30 mL). Combined organics were washed (brine 30 mL), dried (phase sep paper) and concentrated to dryness affording a green film. The combined organics were washed (brine 30 mL), dried (phase sep paper) and concentrated to dryness affording a green film. The film was purified by preparative HPLC automated flash chromatography (ISCO ACCQ HP125, Prep HPLC Column: B/J Column Dimensions: 30 mm×250 mm 5 μM; Sample: 2.50 ml from tube 1 RediSep column) eluting with 5 to 95% ACN/0.1% FA in Water/0.1% FA. The desired fractions were concentrated to dryness affording [1-(5-{[2-methyl-6-(trifluoromethyl)phenyl] methoxy}pyridin-2-yl) imidazol-4-yl]methanol (6 mg, 0.02 mmol, 5.72%) as a white powder (Purity: >95%).

### Example 45

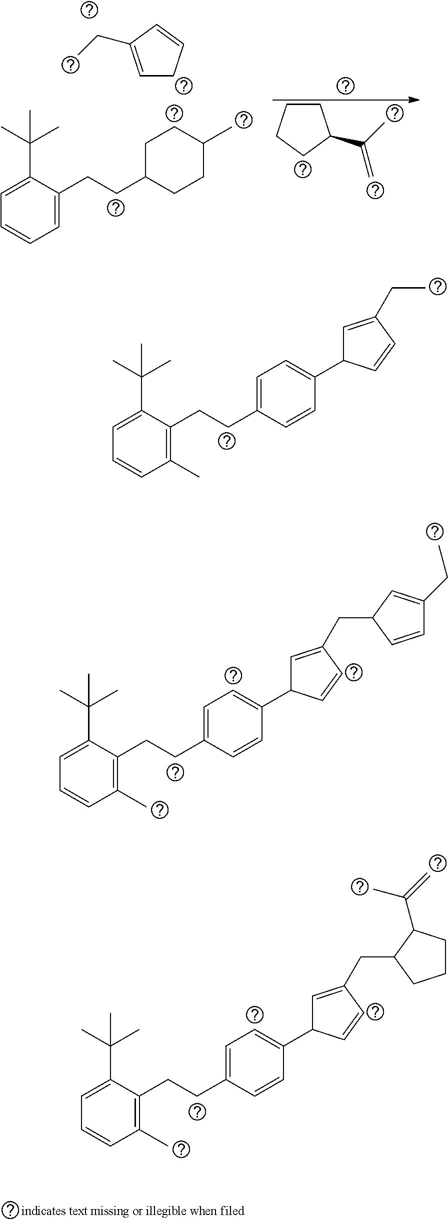

To a degassed stirred solution of 4-(HYDROXYMETHYL) IMIDAZOLE HYDROCHLORIDE (46.65 mg, 0.35 mmol, 1.2 eq), 2-bromo-5-[(2,6-dimethylphenyl) methoxy]pyridine (100 mg, 0.29 mmol, 1 eq), L-PROLINE (4.93 uL, 1.35 g/mL, 0.06 mmol, 0.2 eq) and POTASSIUM CARBONATE (119.78 mg, 0.87 mmol, 3 eq) in DMSO (3 mL) was added COPPER (I) IODIDE (5.5 mg, 0.03 mmol, 0.1 eq). The reaction was sealed and heated to 90° C. for 72 h. The reaction was cooled to rt and filtered through celite before being poured into brine (100 mL) and extracted with ethyl acetate (3×30 mL). Combined organics were washed (brine 30 mL), dried (phase sep paper) and concentrated to dryness affording a green film. The film was purified by preparative HPLC automated flash chromatography (ISCO ACCQ HP125, Prep HPLC Column: B/J Column Dimensions: 30 mm×250 mm 5 μM; Sample: 2.50 ml from tube 1 RediSep column) eluting with 5 to 95% ACN/0.1% FA in Water/0.1% FA. The desired fractions were concentrated to dryness affording [1-(5-{[2-methyl-6-(trifluoromethyl)phenyl] methoxy}pyridin-2-yl) imidazol-4-yl] methanol (6 mg, 0.02 mmol, 5.72%) as a white powder.

### Example 46

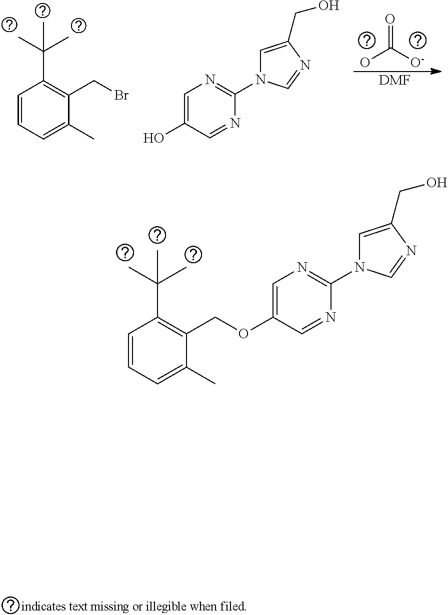

To a degassed stirred solution of 4-(HYDROXYMETHYL) IMIDAZOLE HYDROCHLORIDE (46.52 mg, 0.35 mmol, 1.2 eq), 2-bromo-5-[(2,6-dimethylphenyl) methoxy]pyridine (100 mg, 0.29 mmol, 1 eq), L-PROLINE (4.91 uL, 1.35 g/mL, 0.06 mmol, 0.2 eq) and POTASSIUM CARBONATE (119.44 mg, 0.86 mmol, 3 eq) in DMSO (3 mL) was added COPPER (I) IODIDE (5.49 mg, 0.03 mmol, 0.1 eq). The reaction was sealed and heated to 90° C. for 72 h. The reaction was cooled to rt and poured into brine (100 mL) before being extracted with ethyl acetate (3×30 mL). Combined organics were washed (brine 30 mL), dried (phase sep paper) and concentrated to dryness affording a brown film. The film was purified by preparative HPLC automated flash chromatography (ISCO ACCQ HP125, Prep HPLC Column: B/J Column Dimensions: 30 mm×250 mm 5 μM; Sample: 2.50 ml from tube 1 RediSep column) eluting with 5 to 95% ACN/0.1% FA in Water/0.1% FA. The desired fractions were combined and concentrated to dryness affording [1-(5-{[2-methyl-6-(trifluoromethyl)phenyl] methoxy}pyrimidin-2-yl) imidazol-4-yl]methanol (10 mg, 0.03 mmol, 9.53%) as a white powder (Purity: >95%).

### Example 47

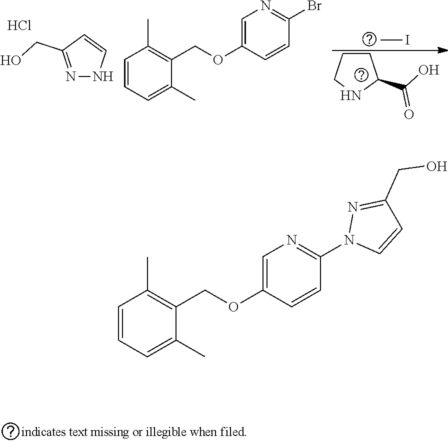

To a degassed stirred solution of 1H-Pyrazole-3-methanol Hydrochloride (55.27 mg, 0.41 mmol, 1.2 eq), 2-bromo-5-[(2,6-dimethylphenyl) methoxy]pyridine (100 mg, 0.34 mmol, 1 eq), L-PROLINE (5.84 uL, 1.35 g/mL, 0.07 mmol, 0.2 eq) and POTASSIUM CARBONATE (141.91 mg, 1.03 mmol, 3 eq) in DMSO (3 mL) was added COPPER (I) IODIDE (6.52 mg, 0.03 mmol, 0.1 eq). The reaction was sealed and heated to 90° C. for 72 h. The reaction was cooled to rt and poured into brine (100 mL) before being extracted with ethyl acetate (3×30 mL). Combined organics were washed (brine 30 mL), dried (phase sep paper) and concentrated to dryness affording a brown film. washed (brine 30 mL), dried (phase sep paper) and concentrated to dryness affording a brown film. The film was purified by preparative HPLC automated flash chromatography (ISCO ACCQ HP125, Prep HPLC Column: B/J Column Dimensions: 30 mm×250 mm 5 μM; Sample: 2.50 ml from tube 1 RediSep column) eluting with 5 to 95% ACN/0.1% FA in Water/0.1% FA. The desired fractions were collected and concentrated to dryness affording an oily gum. To the vial was added 0.5 mL MeCN and Hydrogen chloride in 1,4-dioxane (15 uL, (4 M), 0.06 mmol, 0.18 eq) (1 eq for product) was added. The vial was concentrated to dryness affording (1-{5-[(2,6-dimethylphenyl) methoxy]pyridin-2-yl}pyrazol-3-yl) methanol affording (1-{5-[(2,6-dimethylphenyl) methoxy]pyridin-2-yl}pyrazol-3-yl) methanol (19.3 mg, 0.06 mmol, 18.23%) as a off-white gum (Purity: >95%).

### Example 48

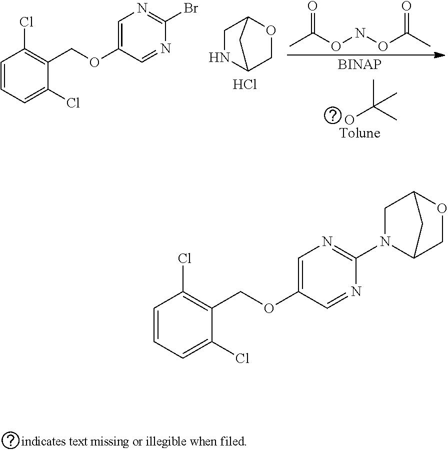

SODIUM TERT-BUTOXIDE (57.55 mg, 0.6 mmol, 4 eq) was added to a stirred solution of 2-bromo-5-[(2,6-dichlorophenyl) methoxy]pyrimidine (50 mg, 0.15 mmol, 1 eq), 2═Oxa-5-azabicyclo[2.2.1]heptane HCl (40.6 mg, 0.3 mmol, 2 eq), Palladium acetate (5.04 mg, 0.02 mmol, 0.15 eq) and BINAP(s)-(−)-2,2′-Bis(diphenylphosphino)-1,1′-binaphthyl (13.98 mg, 0.02 mmol, 0.15 eq) in Toluene (2 mL) under a nitrogen atmosphere. The reaction was heated to reflux, where it was maintained overnight. The reaction was cooled to rt then the solvent removed in vacuo. The resultant residue was dissolved in EtOAc then washed with water (x2) followed by brine. The organic extract was dried (MgSO₄) then the solvent removed in vacuo. The resultant residue was dissolved in DMSO then filtered and purified by preparative HPLC automated flash chromatography (ISCO ACCQ HP150, Prep HPLC Column: B/J column Dimensions: 30 mm×25 mm 5 M; Sample: 1.80 ml from tube 1 RediSep column) eluting with 5 to 95% ACN/0.1% FA in Water/0.1% FA. The desired fractions were combined then freeze dried giving a glass. The glass was dissolved in DCM then the solvent removed under a stream of nitrogen and the compound dried under vacuum at 40° C. over the weekend to give 5-{5-[(2,6-dichlorophenyl) methoxy]pyrimidin-2-yl}-2-oxa-5-azabicyclo[2.2.1]heptane (3.63 mg, 0.01 mmol, 6.88%) as a colourless glass (Purity: >95%).

### Example 49

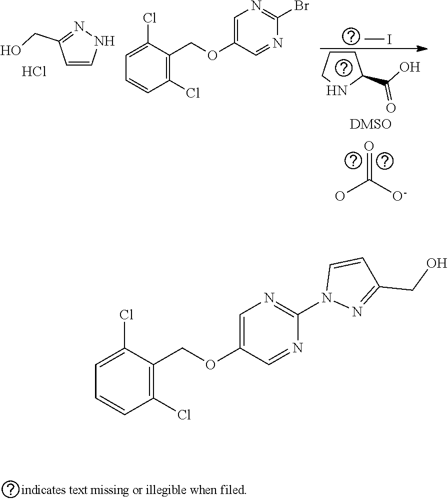

Nitrogen was bubbled through a DMSO (3 mL) solution of 1H-Pyrazole-3-methanol Hydrochloride (48.35 mg, 0.36 mmol, 1.2 eq), 2-bromo-5-[(2,6-dichlorophenyl) methoxy]pyrimidine (100 mg, 0.3 mmol, 1 eq), L-PROLINE (6.89 mg, 0.06 mmol, 0.2 eq) and POTASSIUM CARBONATE (124.14 mg, 0.9 mmol, 3 eq) then Copper (I) iodide (5.7 mg, 0.03 mmol, 0.1 eq) added and the flask sealed. The reaction was heated to 90° C., where it was maintained overnight. LCMS showed product present. The reaction was cooled to rt then the solvent removed in vacuo. The resultant residue was dissolved in EtOAc then washed with water (x2) followed by brine. The organic extract was dried (MgSO₄) then the solvent removed in vacuo. The resultant residue was dissolved in DMSO then filtered and purified by preparative HPLC automated flash chromatography (ISCO ACCQ HP150, Sample: 1.80 ml from tube 1; Prep HPLC Column: B/J column Dimensions: 30 mm×25 mm 5 μM RediSep column) eluting with 5 to 95% ACN/0.1% FA in Water/0.1% FA. The desired fractions were combined then freeze dried to give (1-{5-[(2,6-dichlorophenyl) methoxy]pyrimidin-2-yl}pyrazol-3-yl) methanol (12.72 mg, 0.04 mmol, 12.1%) as a white solid (Purity: >95%).

### Example 50

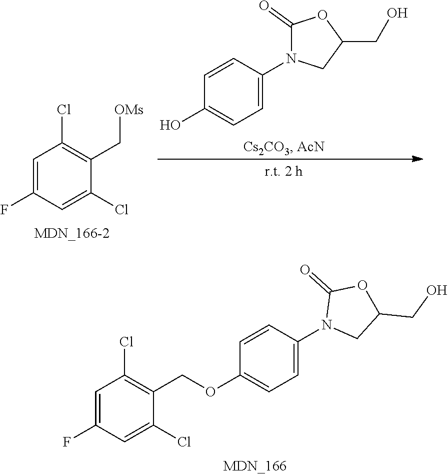

To a mixture of MDN_166-2 (100 mg, 0.360 mmol) in ACN (1 mL) were added intermediate-3 (75 mg, 0.360 mmol) and Cs₂CO₃ (234 mg, 0.720 mmol). The reaction was stirred at r.t. for 2 h. The reaction mixture was diluted with H₂O (30 mL) and extracted with EtOAc (30 mL×3). The organic layer was dried over Na2SO4, filtered and concentrated in vacuo. The residue was purified by flash chromatography (EA:PE=0%˜30%) to afford a white solid (20.0 mg, 14.3% yield).

### Example 51

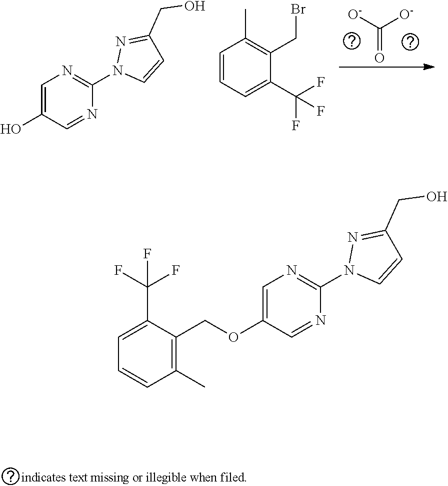

To a stirred solution of 2-[3-(hydroxymethyl) pyrazol-1-yl]pyrimidin-5-ol (170 mg, 0.88 mmol, 1 eq) in DMF (8 mL) under nitrogen was added POTASSIUM CARBONATE (244.52 mg, 1.77 mmol, 2 eq). Mixture stirred for 5 mins before a solution of 2-METHYL-6-TRIFLUOROMETHYLBENZYL BROMIDE (223.86 mg, 0.88 mmol, 1 eq) in DMF (1 mL) was added dropwise. Reaction stirred for 18 h at rt. LCMS showed excellent conversion to product with some bisbenzylated biproduct. Reaction was poured into brine (100 mL) before being extracted with ethyl acetate (2×100 mL). Combined organics were washed (brine 2 x 80 mL), dried (phase sep paper) and concentrated to dryness affording a colourless oil. The oil was purified by automated flash chromatography (Combiflash 300+, Silica 24 g RediSep column) eluting with 0 to 15% Methanol in Dichloromethane Analysis of the fractions showed a mixture of product and other impurities. All fractions containing product were combined and concentrated to dryness affording a colourless gum. The gum was purified by preparative HPLC automated flash chromatography (ISCO ACCQ HP150, Prep HPLC Column: B/J column Dimensions: 30 mm×250 mm 5 μM; Sample: 2.50 ml from tube 1 RediSep column) eluting with 5 to 95% ACN/0.1% FA in Water/0.1% FA. The desired fractions were combined and concentrated to dryness affording [1-(5-{[2-methyl-6-(trifluoromethyl)phenyl] methoxy}pyrimidin-2-yl)pyrazol3-yl]methanol (159.15 mg, 0.44 mmol, 49.38%) as a white powder (Purity: >95%).

### Example 52

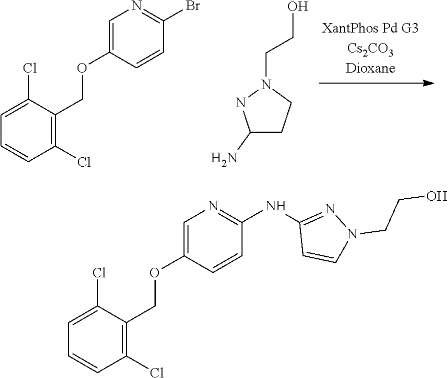 [SMILES: Clc1cccc(Cl)c1COc1ccc(Br)nc1 ; NC1CCN(CCO)N1 ; OCCn1ccc(Nc2ccc(OCc3c(Cl)cccc3Cl)cn2)n1]

To a degassed stirred solution of 2-bromo-5-[(2,6-dichlorophenyl) methoxy]pyridine (100 mg, 0.3 mmol, 1 eq) in 1,4-Dioxane (3 mL) with 2-(3-Amino-1H-pyrazol-1-yl) ethanol (45.82 mg, 0.36 mmol, 1.2 eq) and CESIUM CARBONATE (195.68 mg, 0.6 mmol, 2 eq) was added XantPhos Pd G3 (28.51 mg, 0.03 mmol, 0.1 eq). The reaction stirred for 90 h at 100° C. and was filtered through a plug of celite before being concentrated to dryness affording a brown film. The film was taken into DMSO before being purified by prep-LCMS. The desired fractions were concentrated to dryness affording a brown film. The film was repurified by automated flash chromatography (Combiflash 300+, Silica 12 g RediSep column) eluting with 0 to 100% 20% NH₃. MeOH in Dichloromethane. The desired fractions were concentrated to dryness affording 2-[3-({5-[(2,6-dichlorophenyl) methoxy]pyridin-2-yl}amino) pyrazol-1-yl]ethanol (34.3 mg, 0.09 mmol, 30.12%) as a white powder (Purity: >95%).

### Example 53

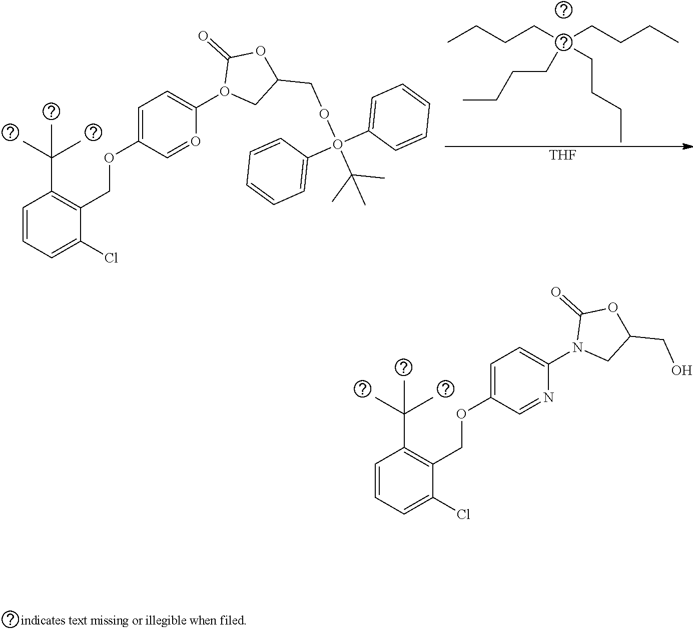

A solution of 5-{[(tert-butyldiphenylsilyl)oxy] methyl}-3-(5-{[2-chloro-6-(trifluoromethyl)phenyl] methoxy}pyridin-2-yl)-1,3-oxazolidin-2-one (40 mg, 0.06 mmol, 1 eq) in THF (5 mL) was cooled in an ice bath then TETRABUTYLAMMONIUM FLUORIDE (1M in THF) (0.01 mL, 1.9 g/mL, (1 M), 0.09 mmol, 1.5 eq) added slowly (reaction turns red). The reaction was stirred in the ice bath for about 15 min then at rt overnight. The reaction was quenched with sat. aq. NaHCO3 solution then extracted with EtOAc (×2). The organic extract was washed with further sat. aq. NaHCO3 solution followed by brine then dried (MgSO₄) and the solvent removed in vacuo. The resultant residue was dissolved in DMSO, filtered then purified by preparative HPLC automated flash chromatography (ISCO ACCQ HP150, Prep HPLC Column: gemini 150×20 5 um Dimensions: 20 mm×150 mm 5 μM; Sample: 1.50 ml from tube 1) eluting with 5 to 95% ACN/0.1% FA in Water/0.1% FA to gave 3-(5-{[2-chloro-6-(trifluoromethyl)phenyl] methoxy}pyridin-2-yl)-5-(hydroxymethyl)-1,3-oxazolidin-2-one (9.14 mg, 0.02 mmol, 36.38%) as a white solid (Purity: >95%).

### Example 54

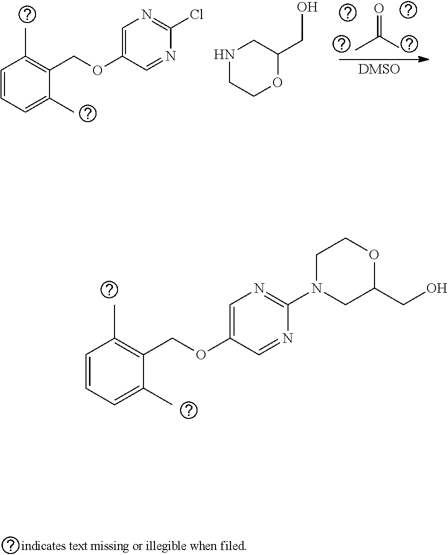

Nitrogen was bubbled through a DMSO (1 mL) solution of 2-chloro-5-[(2,6-dichlorophenyl) methoxy]pyrimidine (50 mg, 0.17 mmol, 1 eq), (morpholin-2-yl) methanol (30.34 mg, 0.26 mmol, 1.5 eq) and POTASSIUM CARBONATE (71.6 mg, 0.52 mmol, 3 eq) in a microwave vial. The vial was capped then heated to 120° C., where it was maintained overnight. The reaction was cooled to rt then diluted with EtOAc and washed with water followed by brine. The organic extract was dried (MgSO₄) then the solvent removed in vacuo. The resultant residue was dissolved in DMSO, filtered then purified by preparative HPLC automated flash chromatography (ISCO ACCQ HP150, Prep HPLC Column: gemini 150×20 5 um Dimensions: 20 mm×150 mm 5 μM; Sample: 1.50 ml from tube 1) eluting with 5 to 95% ACN/0.1% FA in Water/0.1% FA. The desired fractions were combined then freeze dried. Gave (4-{5-[(2,6-dichlorophenyl) methoxy]pyrimidin-2-yl}morpholin-2-yl) methanol (30.09 mg, 0.08 mmol, 47.06%) as a white solid (Purity: >95%).

### Example 55

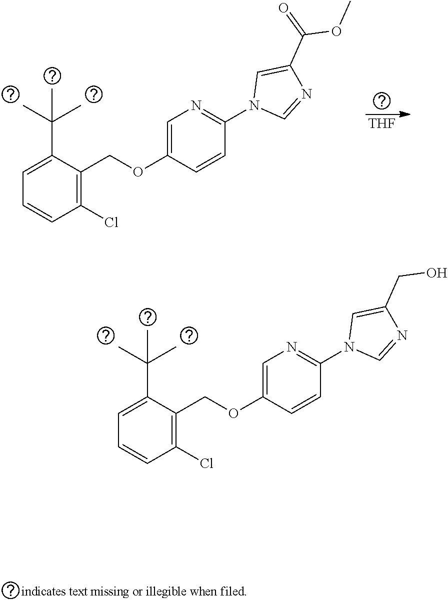 [SMILES: COC(=O)c1cn(-c2ccc(OCc3c(Cl)cccc3C(C)(C)C)cn2)cn1 ; CC(C)(C)c1cccc(Cl)c1COc1ccc(-n2cnc(CO)c2)nc1 ; "THF"]

A solution of methyl 1-(5-{[2-chloro-6-(trifluoromethyl)phenyl] methoxy}pyridin-2-yl) imidazole-4-carboxylate (108 mg, 0.26 mmol, 1 eq) in THF (5 mL) was cooled in an ice bath under a nitrogen atmosphere then LITHIUM ALUMINUM HYDRIDE (1M in THF) (0.52 mL, (1 M), 0.52 mmol, 2 eq) was added slowly. The reaction was stirred in the ice bath for ca 30 min then at rt for a further ca 30 min. The reaction was quenched with water then extracted with EtOAc (×2). The organic extract was washed with sat. aq. NaHCO3 solution followed by brine then dried (MgSO4) and the solvent removed in vacuo. The resultant residue was dissolved in DMSO, filtered, then purified by preparative HPLC automated flash chromatography (ISCO ACCQ HP150, Prep HPLC Column: B/J column Dimensions: 30 mm×25 mm 5 μM; Sample: 1.50 ml from tube 1) eluting with 5 to 95% ACN/0.1% FA in Water/0.1% FA. The desired fractions were freeze dired to give [1-(5-{[2-chloro-6-(trifluoromethyl)phenyl] methoxy}pyridin-2-yl) imidazol-4-yl] methanol (2.96 mg, 0.01 mmol, 2.94%) as a white solid (Purity: >95%).

### Example 56

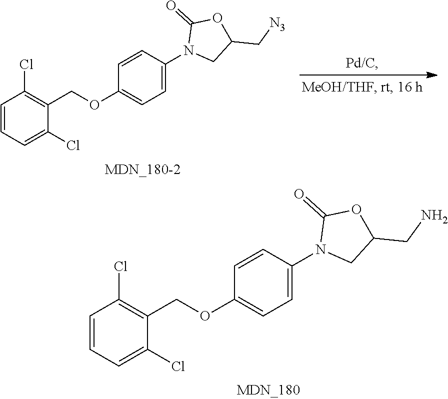 [SMILES: [N-]=[N+]=NCC1CN(c2ccc(OCc3c(Cl)cccc3Cl)cc2)C(=O)O1 ; NCC1CN(c2ccc(OCc3c(Cl)cccc3Cl)cc2)C(=O)O1]

To a solution of MDN_180-2 (260 mg crude, 0.580 mmol) in THF (3 mL) and MeOH (1 mL) was added Pd/C (50 mg). The reaction was stirred at rt for 16 h under H₂ atmosphere. The mixture was filtered through a pad of Celite and rinsed with MeOH. The filtrate was concentrate and the residue was purified by prep-HPLC (TFA) to obtain MDN_180 (30 mg, 14.1% yield, as a white solid).

### Example 57

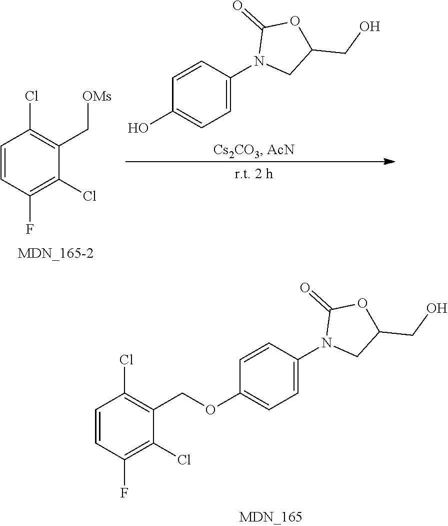

To a mixture of MDN_165-2 (130 mg, 0.480 mmol) in ACN (1 mL) were added intermediate-3 (100 mg, 0.480 mmol) and Cs2CO3 (312 mg, 0.960 mmol). The reaction was stirred at r.t. for 2 h. The reaction mixture was diluted with H₂O (80 mL) and extracted with EtOAc (80 mL×3). The organic layer was dried over Na2SO4, filtered and concentrated in vacuo. The residue was purified by flash chromatography (EA:PE=0%˜50%) to afford a white solid (47 mg, 25.0% yield).

### Example 58

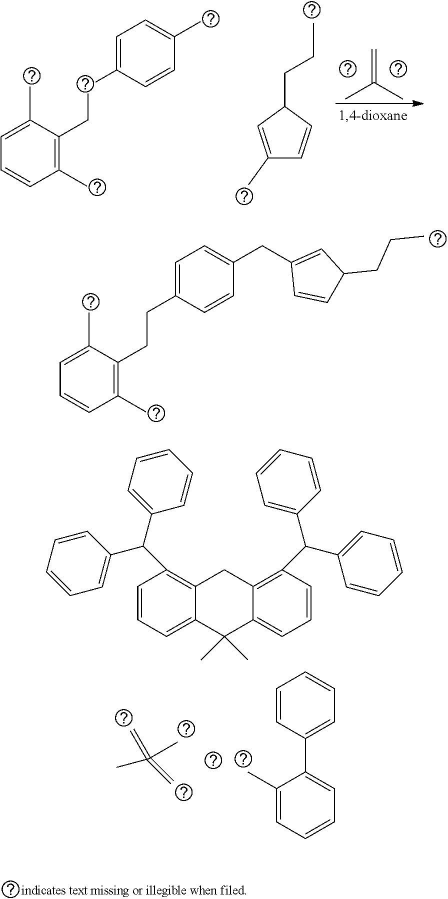

XantPhos Pd G3 (26.55 mg, 0.03 mmol, 0.1 eq) was added to a stirred solution of 2-bromo-5-[(2,6-dichlorophenyl) methoxy]pyridine (93.13 mg, 0.28 mmol, 1 eq), 2-(3-amino-1,2,4-triazol-1-yl) ethanol (43 mg, 0.34 mmol, 1.2 eq) and CAESIUM CARBONATE (182.24 mg, 0.56 mmol, 2 eq) in 1,4-Dioxane (2 mL) under a nitrogen atmosphere. The reaction was heated to 100° C., where it was maintained for about 5 h. The reaction continued to stir at rt overnight. The reaction was diluted with EtOAc then washed with water followed by brine. the organic extract was dried (MgSO₄) then the solvent removed in vacuo. The resultant residue was dissolved in DMSO, filtered then purified by preparative HPLC automated flash chromatography (ISCO ACCQ HP125, Prep HPLC Column: Gemini Dimensions: 21 mm×150 mm 5 μM; Sample: 1.50 ml from tube 1) eluting with 5 to 95% ACN/0.1% FA in Water/0.1% FA. The desired fractions were combined and freeze dried to give 2-[3-({5-[(2,6-dichlorophenyl) methoxy]pyridin-2-yl}amino)-1,2,4-triazol-1-yl]ethanol (7.84 mg, 0.02 mmol, 7.37%) as an off-white solid (Purity: >95%).

### Example 59

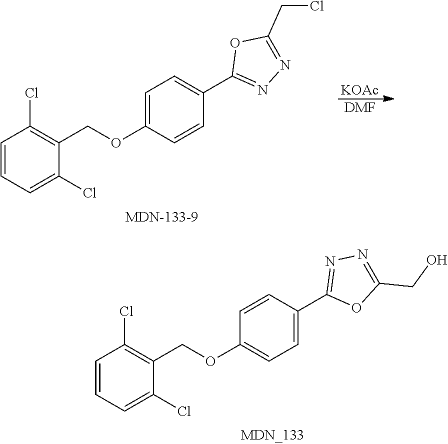

A mixture of MDN_133-9 (100 mg, 0.27 mmol) and KOAc (53 mg, 0.54 mmol) in DMF (3 ml) was stirred at 80oC for 2 h. The mixture was quenched with H2O, extracted with DCM, concentrated to give the residue, which was purified by chromatography on silica gel (EtOAc/PE=0-20%) to give the title compound MDN_133 as a white solid (73 mg, 76% yield).

### Example 60

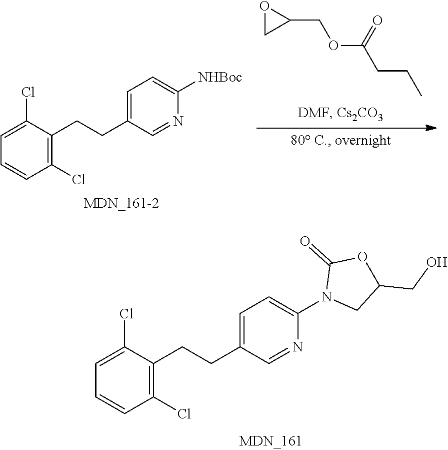

To a mixture of MDN_161-2 (0.186 g, 0.506 mmol) in DMF (5 mL) was added oxiran-2-ylmethyl butyrate (0.218 g, 1.52 mmol) and Cs2CO3 (0.494 g, 1.52 mmol). The reaction was stirred at 80oC overnight. The reaction mixture was diluted with H2O (30 mL) and extracted with EtOAc (30 mL×3). The organic phase was washed with water, dried over Na2SO4 and concentrated. The crude product was purified by silica gel column to give the title compound as a yellow solid (6 mg, 3% yield).

### Example 61

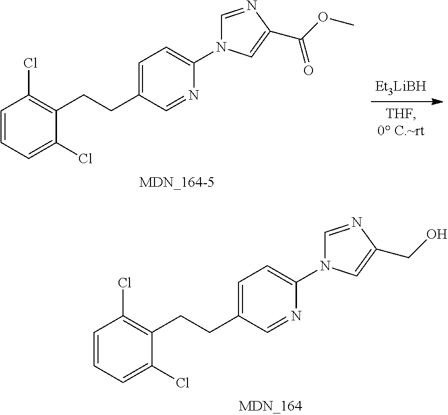 [SMILES: COC(=O)c1cn(-c2ccc(CCc3c(Cl)cccc3Cl)cn2)cn1 ; OCc1cn(-c2ccc(CCc3c(Cl)cccc3Cl)cn2)cn1]

To a solution of MDN_164-5 (60 mg, 0.160 mmol) in THF (1 mL) was added LiBHEt3 (1M, 0.48 mL, 0.480 mmol) slowly at 0° C. The reaction was stirred at rt for 2 h. The reaction mixture was diluted with H₂O (30 mL) and extracted with EA (30 mL×3). The organic layer was dried over Na2SO4, filtered and concentrated. The residue was purified by prep-HPLC (TFA) to obtain MDN_164 (10 mg, 17.9% yield, as a white solid).

### Example 62

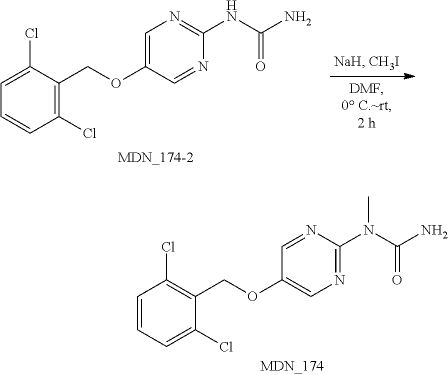 [SMILES: NC(=O)Nc1ncc(OCc2c(Cl)cccc2Cl)cn1 ; CN(C(N)=O)c1ncc(OCc2c(Cl)cccc2Cl)cn1]

To a solution of MDN_174-2 (80 mg, 0.250 mmol) in DMF (1 mL) was added NaH (15 mg, 0.380 mmol) at 0° C. slowly. The mixture was stirred at rt for 0.5 h. Iodomethane (36.0 mg, 0.25 mmol) was added to the mixture. The reaction was stirred at rt for 2 h. The reaction mixture was diluted with H₂O (30 mL) and extracted with EtOAc (30 mL×3). The organic layer was dried over Na2SO4, filtered and concentrated. The residue was purified by prep-HPLC (TFA) to obtain MDN_174 (25 mg, 30.6% yield, as a white solid).

### Example 63

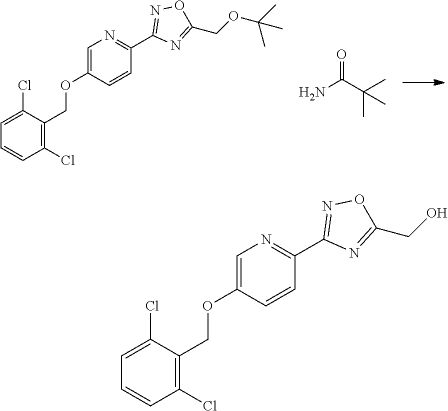

To a stirred solution of 2-[5-(tert-butoxymethyl)-1,2,4-oxadiazol-3-yl]-5-[(2,6-dichlorophenyl) methoxy]pyridine (46.6 mg, 0.11 mmol, 1 eq) in DCM (3 mL) was added TRIFLUOROACETIC ACID (42.53 uL, 1.53 g/mL, 0.57 mmol, 5 eq. The reaction stirred for 72 h. TRIFLUOROACETIC ACID (170.12 uL, 1.53 g/mL, 2.28 mmol, 20 eq) was added and the mixture stirred for a further 2 h. The reaction mixture was concentrated to dryness affording a black oil. The oil was purified by preparative HPLC automated flash chromatography (ISCO ACCQ HP150, Prep HPLC Column: gemini 150x20 5 um Dimensions: 20 mm×150 mm 5 μM; Sample: 2.50 ml from tube 1 RediSep column) eluting with 5 to 95% ACN/0.1% FA in Water/0.1% FA. The desired fractions were concentrated to dryness affording (3-{5-[(2,6-dichlorophenyl) methoxy]pyridin-2-yl}-1,2,4-oxadiazol-5-yl) methanol (18 mg, 0.05 mmol, 44.78%) as a white powder (Purity: >95%).

### Example 64

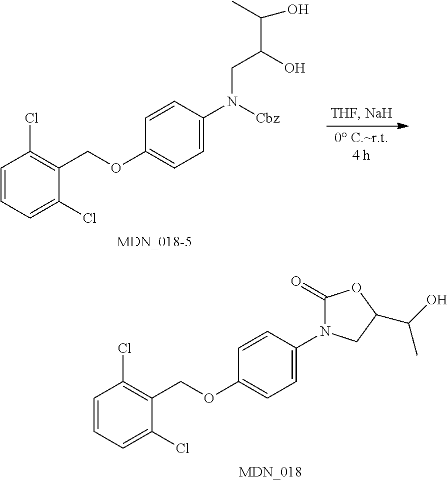 [SMILES: CC(O)C(O)CN(C(=O)OCc1ccccc1)c1ccc(OCc2c(Cl)cccc2Cl)cc1 ; CC(O)C1CN(c2ccc(OCc3c(Cl)cccc3Cl)cc2)C(=O)O1]

To a solution of MDN_018-5 (100 mg, 0.204 mmol) in anhydrous THF (5 mL) was added NaH (12 mg, 0.306 mmol) at 0° C. slowly.

The reaction was stirred at room temperature for 4 h. The reaction mixture was diluted with H₂O (20 mL) and extracted with EtOAc (15 mL×3). The organic layer was dried over Na2SO4, filtered and concentrated in vacuo. The residue was purified by prep-HPLC (NH4HCO3) and lyophilized to afford the title compound (44.4 mg, 56.9% yield).

### Example 65

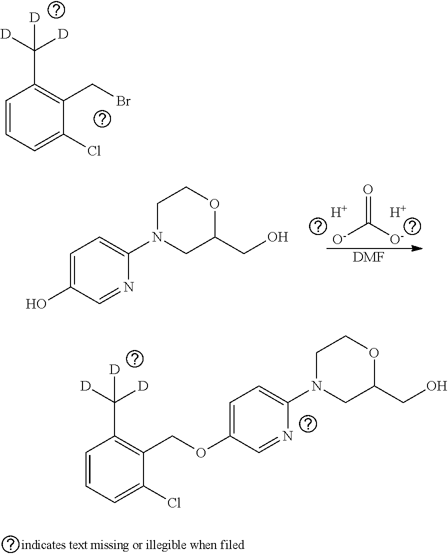

6-[2-(hydroxymethyl) morpholin-4-yl]pyridin-3-ol (16.4 mg, 0.08 mmol, 1 eq) was dissolved in DMF (1 mL), then 2-Chloro-6-(trifluoromethyl)benzyl bromide (21.33 mg, 0.08 mmol, 1 eq) followed by POTASSIUM CARBONATE (53.91 mg, 0.39 mmol, 5 eq) were added. The reaction was stirred at rt for about 2 h. The reaction was diluted with EtOAc then washed with water followed by brine. The organic extract was dried (MgSO₄) then the solvent removed in vacuo. The resultant residue was dissolved in DMSO, filtered then purified by preparative HPLC automated flash chromatography (ISCO ACCQ HP150, Prep HPLC Column: gemini 150×20 5 um Dimensions: 20 mm×150 mm 5 μM; Sample: 1.80 ml from tube 1) eluting with 5 to 95% ACN/0.1% FA in Water/0.1% FA. The desired fractions were combined then freeze dried to give a glass which was dissolved in DCM/MeOH and the solvent removed under a stream of nitrogen. The compound was dried overnight under vacuum at 40° C. to give [4-(5-{[2-chloro-6-(trifluoromethyl)phenyl] methoxy}pyridin-2-yl) morpholin-2-yl] methanol (11.55 mg, 0.03 mmol, 36.76%) as a beige glass (Purity: >95%).

### Example 66

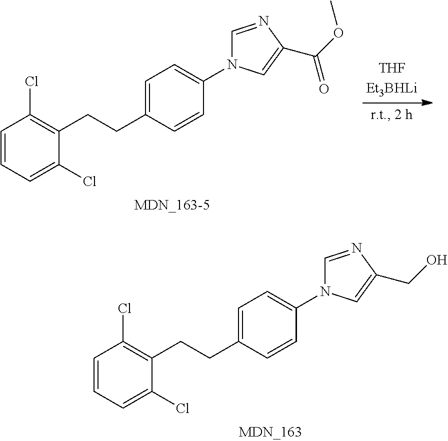 [SMILES: COC(=O)c1cn(-c2ccc(CCc3c(Cl)cccc3Cl)cc2)cn1 ; OCc1cn(-c2ccc(CCc3c(Cl)cccc3Cl)cc2)cn1]

To a mixture of MDN_163-5 (0.080 g, 0.213 mmol) in THF (2 mL) was added Li(Et)3BH (2.13 mL, 2.14 mmol). The reaction mixture was stirred at r.t. for 2 h. The reaction mixture was diluted with H₂O (30 mL) and extracted with EtOAc (50 ml×3). The organic phase dried over Na2SO4, filtered and concentrated. The crude product was purified by silica gel column (EA: PE=0%˜50%) to give the title compound as a yellow solid (0.030 g, 41.0% yield).

### Example 67

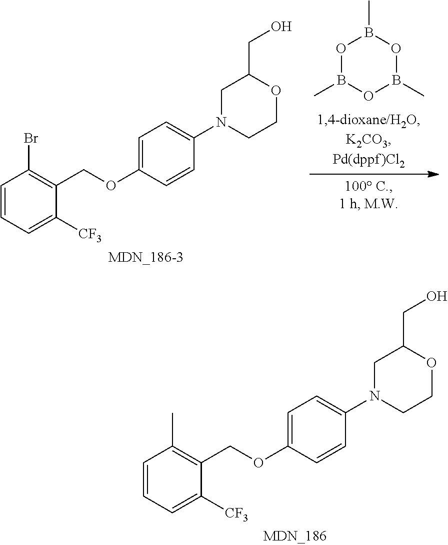

To a solution of MDN_186-3 (26 mg, 0.058 mmol) in 1,4-dioxane (1 mL) and H₂O (0.1 mL) were added 2,4,6-trimethyl-1,3,5,2,4,6-trioxatriborinane (0.5 mL, 0.290 mmol), K2CO3 (24 mg, 0.174 mmol) and Pd(dppf)C12 (8 mg, 0.012 mmol). The reaction was sealed and stirred in a microwave reactor at 100° C. under Ar atmosphere for 1 h. The reaction mixture was diluted with H2O (10 mL) and extracted with EtOAc (20 mL×2). The organic layer was dried over Na2SO4, filtered and concentrated in vacuo. The residue was purified by TLC (PE: EtOAc=1:1) to afford MDN_186 as a white solid (14 mg, 63.3% yield).

### Example 68

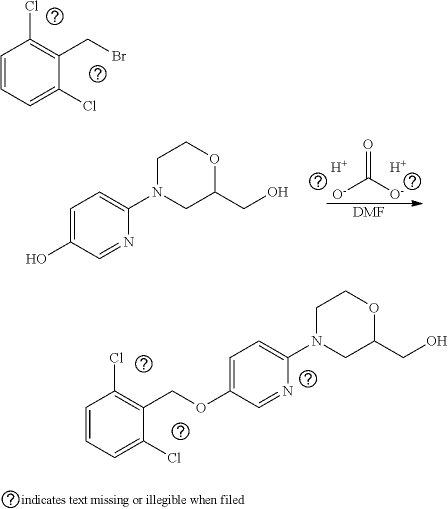

2,6-Dichlorobenzyl bromide (7.19 mg, 0.03 mmol, 1 eq), 6-[2-(hydroxymethyl) morpholin-4-yl]pyridin-3-ol (6.3 mg, 0.03 mmol, 1 eq) and POTASSIUM CARBONATE (20.71 mg, 0.15 mmol, 5 eq) in DMF (1 mL) was stirred under a nitrogen atmosphere at rt over the weekend. The reaction was diluted with EtOAc then washed with water followed by brine. The organic extract was dried (MgSO4) then the solvent removed in vacuo. The resultant residue was dissolved in DMSO, filtered, then purified by preparative HPLC automated flash chromatography (ISCO ACCQ HP150, Prep HPLC Column: gemini 150×20 5 um Dimensions: 20 mm×150 mm 5 μM; Sample: 1.20 ml from tube 1) eluting with 5 to 95% ACN/0.1% FA in Water/0.1% FA. The desired fractions were combined then freeze dried. The resultant glass was dissolved in DCM then transferred to a submission vial and the solvent removed under a stream on nitrogen. The compound was dried overnight under vacuum at 40° C. to give (4-{5-[(2,6-dichlorophenyl) methoxy]pyridin-2-yl}morpholin-2-yl) methanol (3.83 mg, 0.01 mmol, 34.61%) as a colourless glass (Purity: 90-95%).

### Example 69

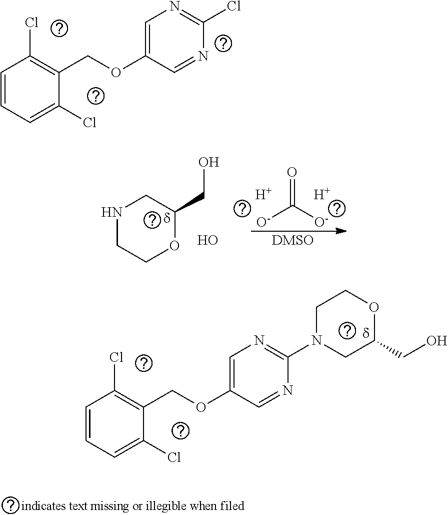

Nitrogen was bubbled through a DMSO (1 mL) solution of 2-chloro-5-[(2,6-dichlorophenyl) methoxy]pyrimidine (50 mg, 0.17 mmol, 1 eq), (S)-2-(Hydroxymethyl) morpholine HCl (39.79 mg, 0.26 mmol, 1.5 eq) and POTASSIUM CARBONATE (71.6 mg, 0.52 mmol, 3 eq) in a microwave vial. The vial was capped then heated to 120° C., where it was maintained for 2 h. The reaction was stirred at rt overnight, diluted with EtOAc then washed with water followed by brine. The organic extract was dried (MgSO₄) then the solvent removed in vacuo. The resultant residue was dissolved in DMSO, filtered and purified by preparative HPLC automated flash chromatography (ISCO ACCQ HP150, Prep HPLC Column: gemini 150×20 5 um Dimensions: 20 mm×150 mm 5 μM; Sample: 1.20 ml from tube 1) eluting with 5 to 95% ACN/0.1% FA in Water/0.1% FA. The desired fractions were collected and freeze dried to give [(2S)-4-{5-[(2,6-dichlorophenyl) methoxy]pyrimidin-2-yl}morpholin-2-yl]methanol (18.6 mg, 0.05 mmol, 29.09%) as a white solid (Purity: >95%).

### Example 70

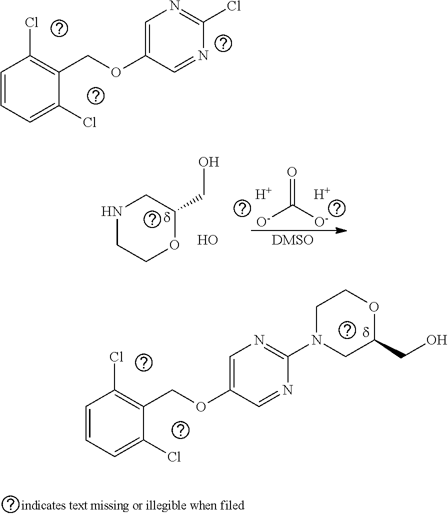

Nitrogen was bubbled through a DMSO (1 mL) solution of 2-chloro-5-[(2,6-dichlorophenyl) methoxy]pyrimidine (50 mg, 0.17 mmol, 1 eq), (R)-2-Hydroxymethylmorpholine HCl (39.79 mg, 0.26 mmol, 1.5 eq) and POTASSIUM CARBONATE (71.6 mg, 0.52 mmol, 3 eq) in a microwave vial. The vial was capped then heated to 120° C., where it was maintained for about 2 h. The reaction was stirred at rt overnight, diluted with EtOAc then washed with water followed by brine. The organic extract was dried (MgSO4) then the solvent removed in vacuo. The resultant residue was dissolved in DMSO, filtered and purified by preparative HPLC automated flash chromatography (ISCO ACCQ HP150, Sample: 1.20 ml from tube 1; Prep HPLC Column: gemini 150×20 5 um Dimensions: 20 mm×150 mm 5 μM) eluting with 5 to 95% ACN/0.1% FA in Water/0.1% FA HP150, Sample: 1.20 ml from tube 1; Prep HPLC Column: gemini 150×20 5 um Dimensions: 20 mm×150 mm 5 μM) eluting with 5 to 95% ACN/0.1% FA in Water/0.1% FA. The desired fractions were freeze dried to give [(2R)-4-{5-[(2,6-dichlorophenyl) methoxy]pyrimidin-2-yl}morpholin-2-yl]methanol (15.09 mg, 0.04 mmol, 23.6%) as a white solid (Purity: >95%).

### Example 71

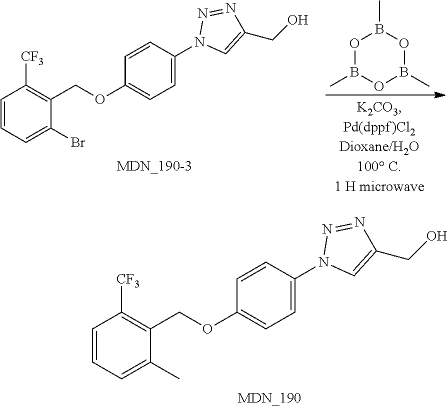

A mixture of MDN_190-3 (60 mg, 0.14 mmol), 2, 4, 6-trimethyl-1, 3, 5, 2, 4, 6-trioxatriborinane (3.5M in THF, 0.2 mL, 0.7 mmol), Pd (dppf)₂Cl₂ (21 mg, 0.028 mmol), K2CO3 (58 mg, 0.42 mmol) in Dioxane/H2O (1 mL/0.2 mL) was stirred at 100° C. in microwave for 1 h. The mixture was quenched with H₂O, extracted with EA, concentrated to give the residue, which was purified by silica gel column (DCM:MeOH=30:1) to get to give the title compound MDN_190 as a brown solid (17 mg, 33% yield).

### Example 72

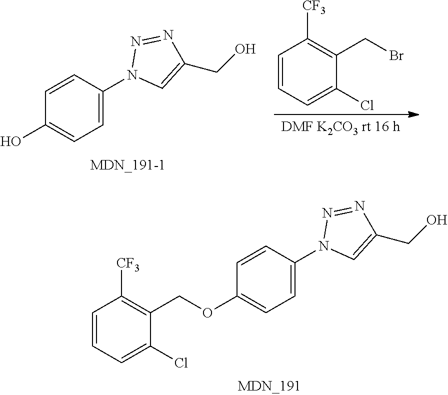

A mixture of MDN_191-1 (50 mg, 0.26 mmol), 2-(bromomethyl)-1-chloro-3-(trifluoromethyl)benzene (72 mg, 0.26 mmol) and K₂CO₃ (54 mg, 0.39 mmol) in DMF (1 mL) was stirred at rt for 16 h. The mixture was quenched with H₂O, filtered to give the title compound MDN_191 as a brown solid (83 mg, 83% yield).

### Example 73

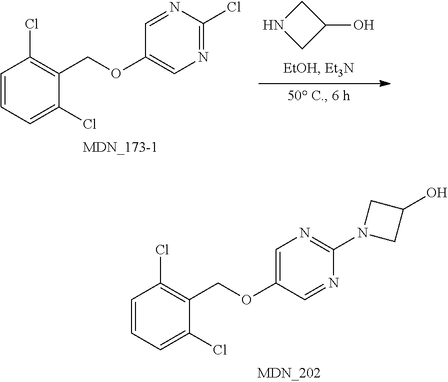 [SMILES: Clc1ncc(OCc2c(Cl)cccc2Cl)cn1 ; OC1CNC1 ; OC1CN(c2ncc(OCc3c(Cl)cccc3Cl)cn2)C1]

To a mixture of MDN_173-1 (80 mg, 0.276 mmol) in EtOH (2 mL) were added azetidin-3-ol (30 mg, 0.276 mmol) and Et₃N (83 mg, 0.828 mmol). The reaction was stirred at 50oC under Ar for 6 h. The reaction mixture was diluted with H₂O (30 mL) and extracted with EtOAc (30 mL×3). The organic phase was dried over Na2SO4 and concentrated. The crude product was purified by silica gel column, eluted with PE: EtOAc=1:1 to give the title compound as a yellow solid (5.6 mg, 6% yield).

### Example 74

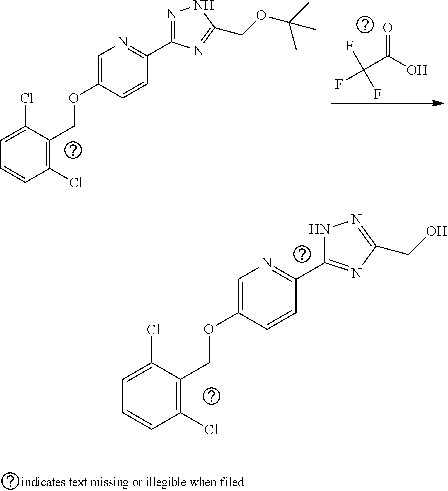

To a stirred solution of 2-[5-(tert-butoxymethyl)-1H-1,2,4-triazol-3-yl]-5-[(2,6-dichlorophenyl) methoxy]pyridine (46.5 mg, 0.11 mmol, 1 eq) in DCM (3 mL) was added TRIFLUOROACETIC ACID (170.17 uL, 1.53 g/mL, 2.28 mmol, 20 eq). Reaction stirred for 18 h at rt. LCMS showed no conversion to product. TRIFLUOROACETIC ACID (170.17 uL, 1.53 g/mL, 2.28 mmol, 20 eq) was added and the reaction stirred for 2 h. The reaction was allowed to stir for a further 18 h. The reaction was concentrated to dryness affording a pale brown film. The film was purified by preparative HPLC automated flash chromatography (ISCO ACCQ HP150, Prep HPLC Column: gemini 150x20 5 um Dimensions: 20 mm×150 mm 5 μM; Sample: 2.50 ml from tube 1 RediSep column) eluting with 5 to 95% ACN/0.1% FA in Water/0.1% FA. The desired fractions were combined and concentrated to dryness affording (5-{5-[(2,6-dichlorophenyl) methoxy]pyridin-2-yl}-1H-1,2,4-triazol-3-yl) methanol (12.2 mg, 0.03 mmol, 30.43%) as a white powder (Purity: >95%).

### Example 75

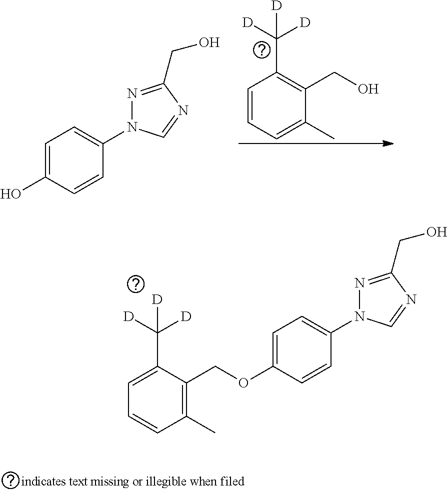 [SMILES: OCc1ncn(-c2ccc(O)cc2)n1 ; [2H]C([2H])([2H])c1cccc(C)c1CO ; [2H]C([2H])([2H])c1cccc(C)c1COc1ccc(-n2cnc(CO)n2)cc1]

To a suspension of 3555-AF-023-001 (100 mg, 0.52 mmol, 1 eq) in DMF (2 mL) was added 2-METHYL-6-TRIFLUOROMETHYLBENZYL BROMIDE (82.73 uL, 1.6 g/cm³, 0.52 mmol, 1 eq) and potassium carbonate (144.57 mg, 1.05 mmol, 2 eq) at rt under nitrogen and the reaction mixture was stirred overnight. The reaction mixture was filtered, washed with DMSO (1 mL) and purified by preparative HPLC automated chromatography (ISCO ACCQ HP125, Sample: 2×1.90 ml from tube 1; Prep HPLC Column: Big Gemini-NX C18 Dimensions: 30 mm×250 mm 5 μM) eluting with 10 to 95% ACN/0.1% FA in Water/0.1% The desired fractions were concentrated to give [1-(4-{[2-methyl-6-(trifluoromethyl)phenyl] methoxy}phenyl)-1,2,4-triazol-3-yl]methanol (88 mg, 0.24 mmol, 46.31%) white solid (Purity: >98%).

### Example 76

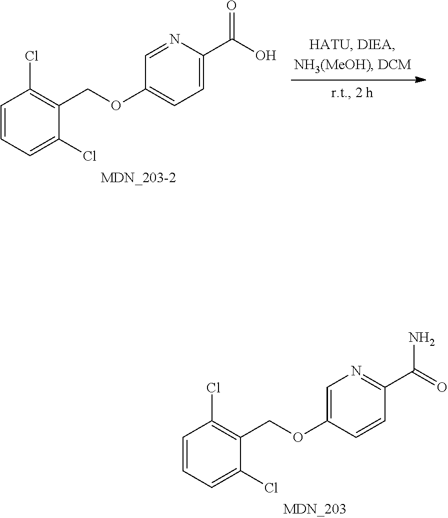 [SMILES: O=C(O)c1ccc(OCc2c(Cl)cccc2Cl)cn1 ; NC(=O)c1ccc(OCc2c(Cl)cccc2Cl)cn1]

To a mixture of MDN_203-2 (0.147 g, 0.494 mmol) in DCM (4 mL) were added 1-[Bis (dimethylamino)methylene]-1H-1,2,3-triazolo[4,5-b]pyridinium-3-oxide hexafluorophosphate (HATU) (0.187 g, 0.494 mmol), diisopropylethylamine (137 mg, 0.988 mmol) and NH₃ (MeOH) (0.5 mL, 0.988 mmol). The reaction mixture was stirred at r.t. under Ar for 2 h. The reaction mixture was diluted with H₂O (20 mL) and extracted with DCM (20 mL×3). The organic phase dried over Na2SO4 and concentrated. The crude product was purified by silica gel column to give the title compound as a yellow solid (20 mg, 14% yield).

### Example 77

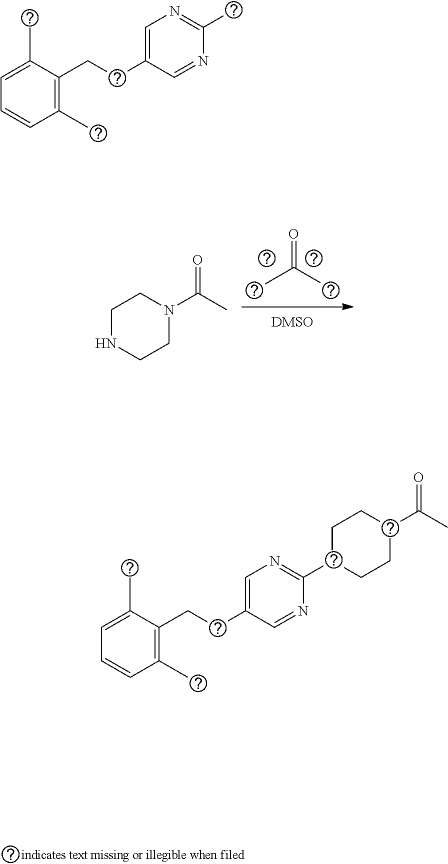

Nitrogen was bubbled through a DMSO (2 mL) solution of 2-chloro-5-[(2,6-dichlorophenyl) methoxy]pyrimidine (100 mg, 0.35 mmol, 1 eq), 1-ACETYLPIPERAZINE (66.4 mg, 0.52 mmol, 1.5 eq) and POTASSIUM CARBONATE (143.2 mg, 1.04 mmol, 3 eq) in a microwave vial. The vial was capped then heated to 120° C., where it was maintained for ca 2.5 h. The reaction was cooled to rt then diluted with EtOAc and washed with water followed by brine. The organic extract was dried (MgSO4) then the solvent removed in vacuo. The resultant residue was dissolved in DMSO, filtered then purified by preparative HPLC automated flash chromatography (ISCO ACCQ HP150, Prep HPLC Column: B/J column Dimensions: 30 mm×250 mm 5 μM; Sample: 1.50 ml from tube 1) eluting with 5 to 95% ACN/0.1% FA in Water/0.1% FA. The desired fractions were combined then freeze dried to give 1-(4-{5-[(2,6-dichlorophenyl) methoxy]pyrimidin-2-yl}piperazin-1-yl) ethanone (44.22 mg, 0.12 mmol, 33.58%) as a white solid (Purity: >95%).

### Example 78

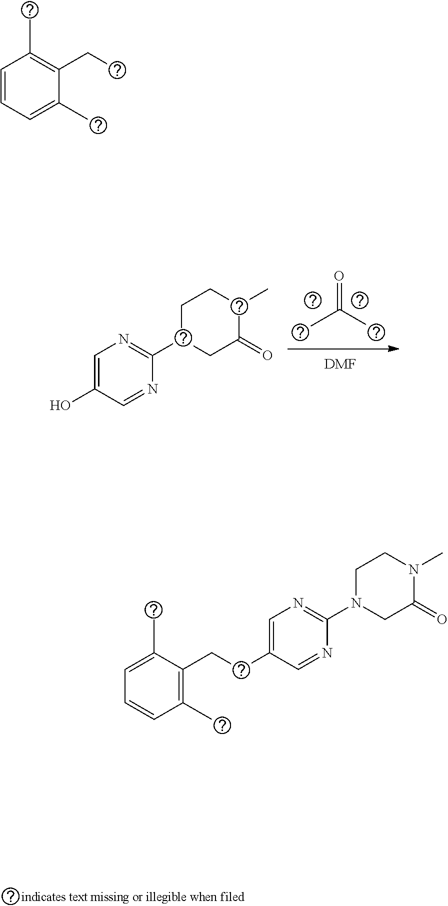

A solution of 2,6-Dichlorobenzyl bromide (24.2 mg, 0.1 mmol, 1 eq), 4-(5-hydroxypyrimidin-2-yl)-1-methylpiperazin-2-one (21 mg, 0.1 mmol, 1 eq) and POTASSIUM CARBONATE (69.69 mg, 0.5 mmol, 5 eq) in DMF (1 mL) was stirred at rt overnight. The reaction was diluted with EtOAc then washed with water followed by brine. The organic extract was dried (MgSO₄) then the solvent removed in vacuo. DMSO (1 ml) was added in order to purify by ACCQ prep however the compound remained partially undissolved. EtOAc was added then the solution washed with water followed by brine. The organic extract was dried (MgSO₄) then the solvent removed in vacuo. The resultant solid was triturated in diethyl ether then collected by filtration to give 4-{5-[(2,6-dichlorophenyl) methoxy]pyrimidin-2-yl}-1-methylpiperazin-2-one (19.73 mg, 0.05 mmol, 53.27%) as a white solid (Purity: >95%).

### Example 79

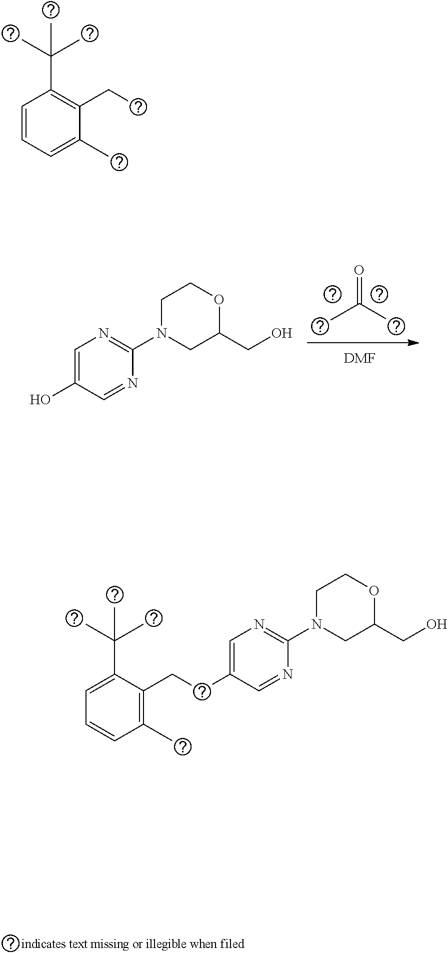

2-Chloro-6-(trifluoromethyl)benzyl bromide (51.79 mg, 0.19 mmol, 1 eq), 2-[2-(hydroxymethyl) morpholin-4-yl]pyrimidin-5-ol (40 mg, 0.19 mmol, 1 eq) and POTASSIUM CARBONATE (130.87 mg, 0.95 mmol, 5 eq) in DMF (2 mL) were stirred at rt for ca 4 days. The reaciton was diluted with EtOAc then washed with water followed by brine. The organic extract was dried (MgSO₄) then the solvent removed in vacuo. The resultant residue was dissolved in DMSO, filtered then purified by preparative HPLC automated flash chromatography (ISCO ACCQ HP150, Prep HPLC Column: Gemini-NX C18 Dimensions: 21 mm×150 mm 5 μM; Sample: 1.50 ml from tube 1) eluting with 5 to 95% ACN/0.1% FA in Water/0.1% FA. The desired fractions were combined then the MeCN removed in vacuo. The aqueous layer was extracted with EtOAc (×2) then the organic extract dried (MgSO₄) and the solvent removed in vacuo. The resultant residue was transferred to a vial using DCM then the solvent removed under a stream of nitrogen. The sample was dried at 40° C. under vacuum overnight to give [4-(5-{[2-chloro-6-(trifluoromethyl)phenyl] methoxy}pyrimidin-2-yl) morpholin-2-yl]methanol (27.5 mg, 0.07 mmol, 35.96%) as a colourless gum (Purity: >95%).

### Example 80

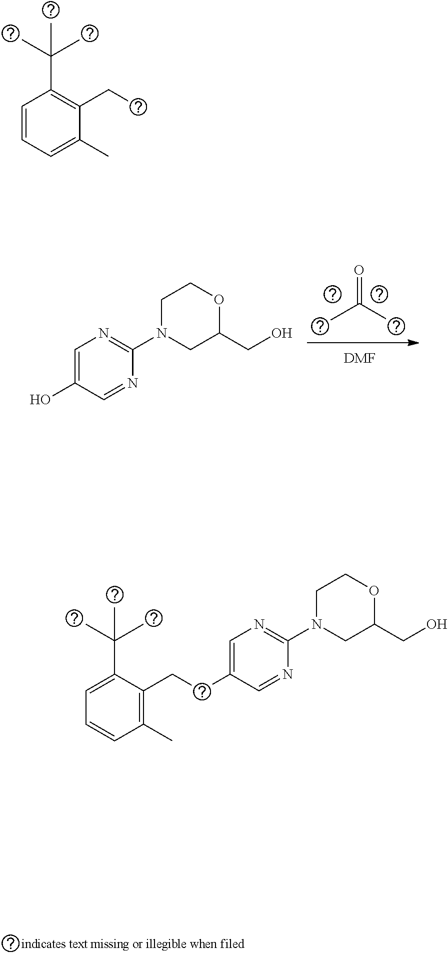

2-METHYL-6-TRIFLUOROMETHYLBENZYL BROMIDE, (47.92 mg, 0.19 mmol, 1 eq), 2-[2-(hydroxymethyl) morpholin-4-yl]pyrimidin-5-ol (40 mg, 0.19 mmol, 1 eq) and POTASSIUM CARBONATE (130.87 mg, 0.95 mmol, 5 eq) in DMF (2 mL) were stirred at rt for ca 4 days. The reaciton was diluted with EtOAc then washed with water followed by brine. The organic extract was dried (MgSO₄) then the solvent removed in vacuo. The resultant residue was dissolved in DMSO, filtered then purified by preparative HPLC automated flash chromatography (ISCO ACCQ HP150, Sample: 1.50 ml from tube 1; Prep HPLC Column: Gemini-NX C18 Dimensions: 21 mm×150 mm 5 μM) eluting with 5 to 95% ACN/0.1% FA in Water/0.1% FA. The acetonitrile was removed in vacuo from the desired fraction. The remaining aqueous phase was extracted with EtOAc (×2) then the organic extract dried (MgSO₄) and the solvent removed in vacuo. The resultant residue was transferred to a vial using DCM then the solvent removed under a stream of nitrogen. The sample was dried at 40° C. under vacuum overnight to give [4-(5-{[2-methyl-6-(trifluoromethyl)phenyl] methoxy}pyrimidin-2-yl) morpholin-2-yl]methanol (19.2 mg, 0.05 mmol, 26.45%) as a colourless gum (Purity: >95%).

### Example 81

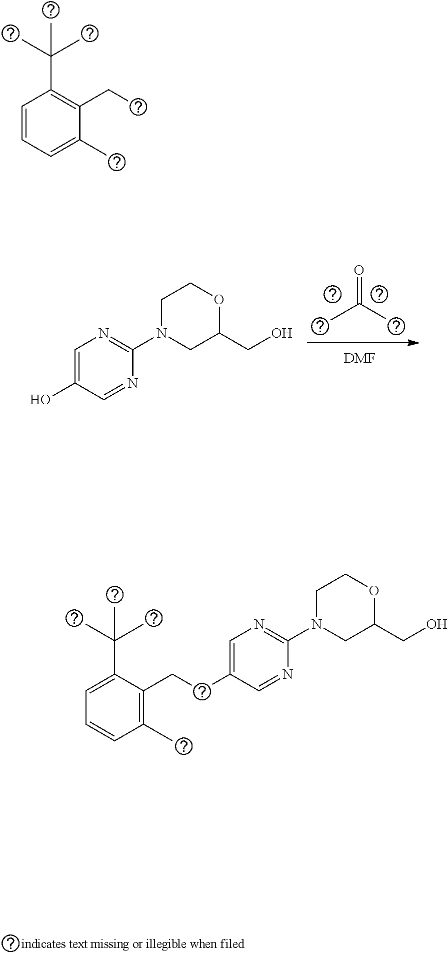

A solution of 2-FLUORO-6-(TRIFLUOROMETHYL) BENZYL BROMIDE (80.31 mg, 0.31 mmol, 1 eq), 2-[2-(hydroxymethyl) morpholin-4-yl]pyrimidin-5-ol (66 mg, 0.31 mmol, 1 eq) and POTASSIUM CARBONATE (215.93 mg, 1.56 mmol, 5 eq) in DMF (2 mL) was stirred at rt overnight. The reaction was diluted with EtOAc then washed with water followed by brine. The organic extract was dried (MgSO4) then the solvent removed in vacuo. The resultant residue was dissolved in DMSO, filtered then purified by preparative HPLC automated chromatography (ISCO ACCQ HP150, Prep HPLC Column: Gemini-NX C18 Dimensions: 21 mm×150 mm 5 μM; Sample: 1.50 ml from tube 1) eluting with 5 to 95% ACN/0.1% FA in Water/0.1% FA. The desired fractions were combined then the MeCN removed in vacuo. The aqueous phase was extracted with EtOAc (x2) then the organic extract dried (MgSO₄) and the solvent removed in vacuo. The resultant residue was transferred to a submission vial usinf DCM then the solvent removed under a stream of nitrogen. The compound was dried overnight at 40° C. under vacuum to give [4-(5-{[2-fluoro-6-(trifluoromethyl)phenyl] methoxy}pyrimidin-2-yl) morpholin-2-yl]methanol (43.4 mg, 0.11 mmol, 35.86%) as a colourless gum (Purity: >95%).

### Example 82

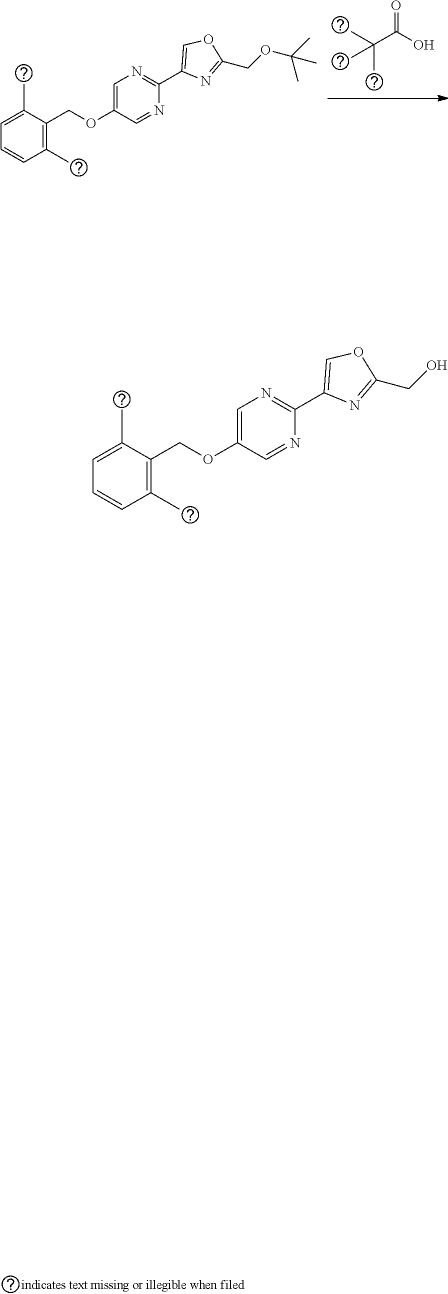

To a stirred solution of 2-[2-(tert-butoxymethyl)-1,3-oxazol-4-yl]-5-[(2,6-dichlorophenyl) methoxy]pyrimidine (37.7 mg, 0.09 mmol, 1 eq) in DCM (2 mL) was added TRIFLUOROACETIC ACID (137.63 uL, 1.53 g/mL, 1.85 mmol, 20 eq). The reaction was stirred for 18 h at rt. Additional TRIFLUOROACETIC ACID (137.63 uL, 1.53 g/mL, 1.85 mmol, 20 eq) was added and the mixture stirred for a further 2 h and then was heated to 40° C. for 1 h. The reaction was cooled to rt and concentrated to dryness affording a yellow oil. The oil was purified by preparative HPLC automated flash chromatography (ISCO ACCQ HP150, Prep HPLC Column: Gemini-NX C18 Dimensions: 21 mm×150 mm 5 μM; Sample: 2.50 ml from tube 1 RediSep column) eluting with 5 to 95% ACN/0.1% FA in Water/0.1% FA. The desired fractions were combined and concentrated to dryness affording (4-{5-[(2,6-dichlorophenyl) methoxy]pyrimidin-2-yl}-1,3-oxazol-2-yl) methanol (1.11 mg, 3.15 μmol, 3.41%) as a white solid (Purity: >95%).

### Example 83

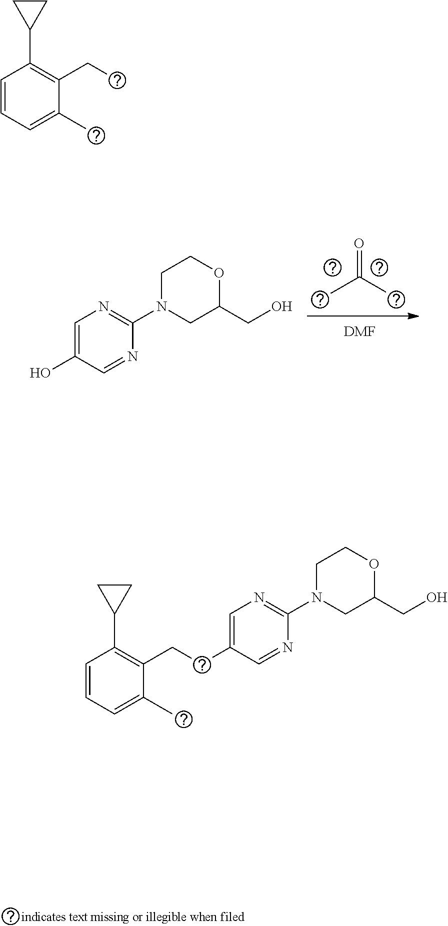

A solution of 2-(bromomethyl)-1-chloro-3-cyclopropylbenzene (41 mg, 0.17 mmol, 1.01 eq), 2-[2-(hydroxymethyl) morpholin-4-yl]pyrimidin-5-ol (35 mg, 0.17 mmol, 1 eq) and POTASSIUM CARBONATE (114.51 mg, 0.83 mmol, 5 eq) in DMF (2 mL) was stirred at rt over the weekend. The reaction was diluted with EtOAc then washed with water followed by brine. The organic extract was dried (MgSO4) then the solvent removed in vacuo. The resultant residue was dissolved in DMSO, filtered then purified by preparative HPLC automated chromatography (ISCO ACCQ HP150, Prep HPLC Column: Gemini-NX C18 Dimensions: 21 mm×150 mm 5 μM; Sample: 1.30 ml from tube 1) eluting with 5 to 95% ACN/0.1% FA in Water/0.1% FA. The desired fractions were combined then freeze dried. The resultant gum was transferred to a vial using DCM. The solvent was removed under a stream on nitrogen then the sample dried overnight at 40° C. under vacuum to give (4-{5-[(2-chloro-6-cyclopropylphenyl) methoxy]pyrimidin-2-yl}morpholin-2-yl) methanol (22.97 mg, 0.06 mmol, 36.88%) as a colourless gum (Purity: >95%).

### Example 84

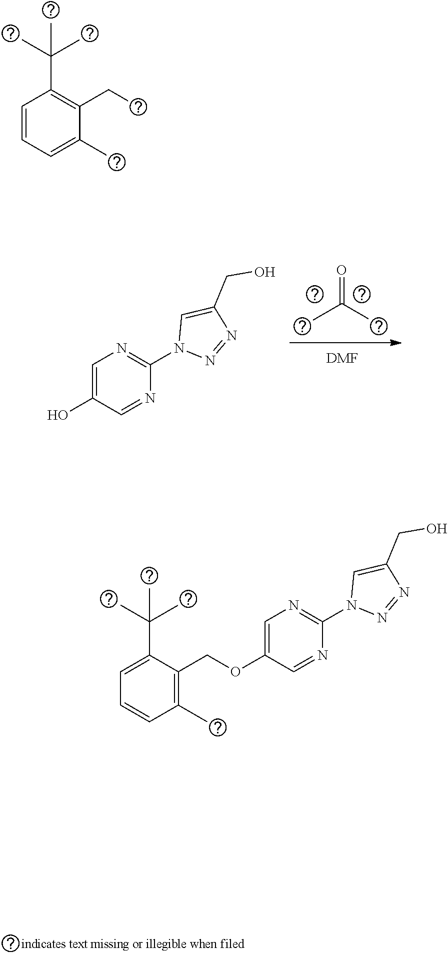

A solution of 2-Chloro-6-(trifluoromethyl)benzyl bromide (18.69 mg, 0.07 mmol, 1 eq), 2-[4-(hydroxymethyl)-1,2,3-triazol-1-yl]pyrimidin-5-ol (13.2 mg, 0.07 mmol, 1 eq) and POTASSIUM CARBONATE (14.17 mg, 0.1 mmol, 1.5 eq) in DMF (1 mL) was stirred at rt for ca 2 h. LCMS shows the reaction has gone to completion The reaction was filtered then purified by preparative HPLC automated chromatography (ISCO ACCQ HP150, Prep HPLC Column: Gemini-NX C18 Dimensions: 21 mm×150 mm 5 μM; Sample: 1.50 ml from tube 1) eluting with 5 to 95% ACN/0.1% FA in Water/0.1% FA. Fraction 17 was freeze dried. Gave [1-(5-{[2-chloro-6-(trifluoromethyl)phenyl] methoxy}pyrimidin-2-yl)-1,2,3-triazol-4-yl] methanol (9.36 mg, 0.02 mmol, 35.51%) as a white solid (Purity: >95%).

### Example 85

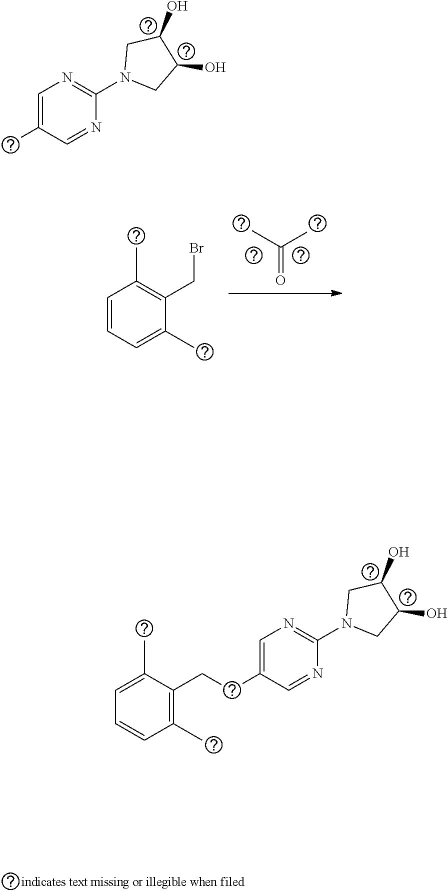

To a stirred solution of (3R,4S)-1-(5-hydroxypyrimidin-2-yl) pyrrolidine-3,4-diol (26.2 mg, 0.13 mmol, 1 eq) in DMF (2 mL) was added 2,6-Dichlorobenzyl bromide (31.87 mg, 0.13 mmol, 1 eq) and POTASSIUM CARBONATE (40.39 mg, 0.29 mmol, 2.2 eq). Reaction stirred for 18 h at rt. LCMS showed excellent conversion to product. The reaction mixture was poured into brine (50 mL) before being extracted with ethyl acetate (3×30 mL). Combined organics were washed (brine 3×20 mL), dried (phase sep paper) and concentrated to dryness affording a brown film. The film was purified by preparative HPLC automated flash chromatography (ISCO ACCQ HP150, Prep HPLC Column: Gemini-NX C18 Dimensions: 21 mm×150 mm 5 μM; Sample: 2.50 ml from tube 1 RediSep column) eluting with 5 to 95% ACN/0.1% FA in Water/0.1% FA Desired fractions were combined and concentrated to dryness affording (3R,4S)-1-{5-[(2,6-dichlorophenyl) methoxy]pyrimidin-2-yl}pyrrolidine-3,4-diol (14 mg, 0.04 mmol, 29.59%) as a colourless gum (Purity: >95%).

### Example 86

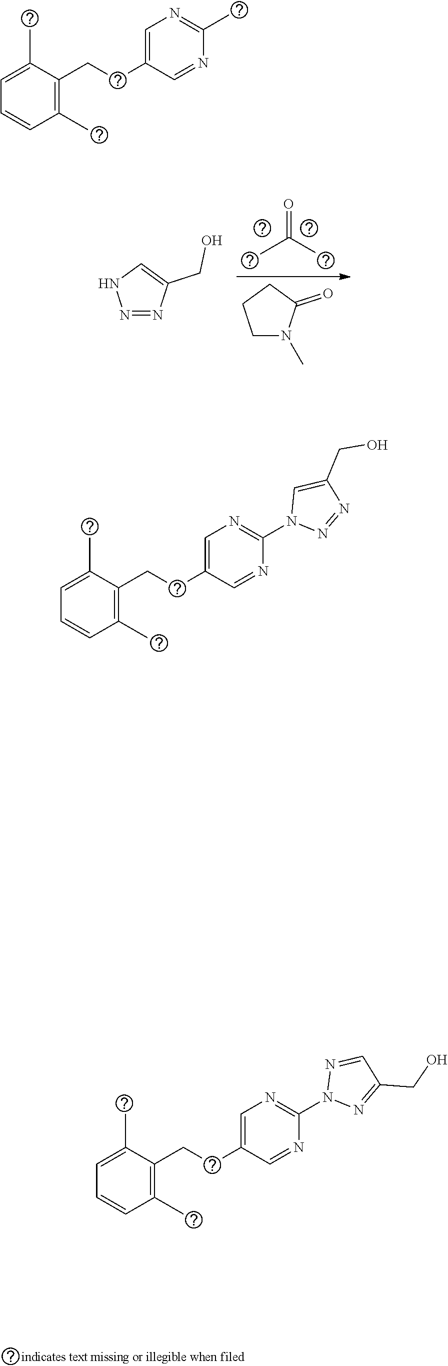

A solution of 2-chloro-5-[(2,6-dichlorophenyl) methoxy]pyrimidine (50 mg, 0.17 mmol, 1 eq), 1H-1,2,3-triazol-4-ylmethanol (17.11 mg, 0.17 mmol, 1 eq) and POTASSIUM CARBONATE (47.73 mg, 0.35 mmol, 2 eq) in 1-METHYL-2-PYRROLIDINONE (1 mL, 1.02 g/mL, 10.29 mmol, 59.58 eq) was heated to 80oC, where it was maintained overnight. The reaction was poured on to water then extracted with EtOAc. The organic extract was washed with brine then dried (MgSO₄) and the solvent removed in vacuo. The resultant residue was dissolved in DMSO then filtered and purified by preparative HPLC automated chromatography (ISCO ACCQ HP150, Prep HPLC Column: Gemini-NX C18 Dimensions: 21 mm×150 mm 5 μM; Sample: 1.40 ml from tube 1) eluting with 5 to 95% ACN/0.1% FA in Water/0.1% FA Fractions 15 and 16 both gave the correct mass but with different retention times by LCMS (0.908 min for 15 (shoulder to peak) and 0.926 for fraction 16). The samples were freeze dried separately. Fraction 15 (6.36 mg) gave (2-{5-[(2,6-dichlorophenyl) methoxy]pyrimidin-2-yl}-1,2,3-triazol-4-yl) methanol (6.36 mg, 0.02 mmol, 10.46%) as a white solid (Purity: >95%). Fraction 16 (6.08 mg) gave (1-{5-[(2,6-dichlorophenyl) methoxy]pyrimidin-2-yl}-1,2,3-triazol-4-yl) methanol (6.08 mg, 0.02 mmol, 10%) as a white solid (Purity: >95%).

### Example 87

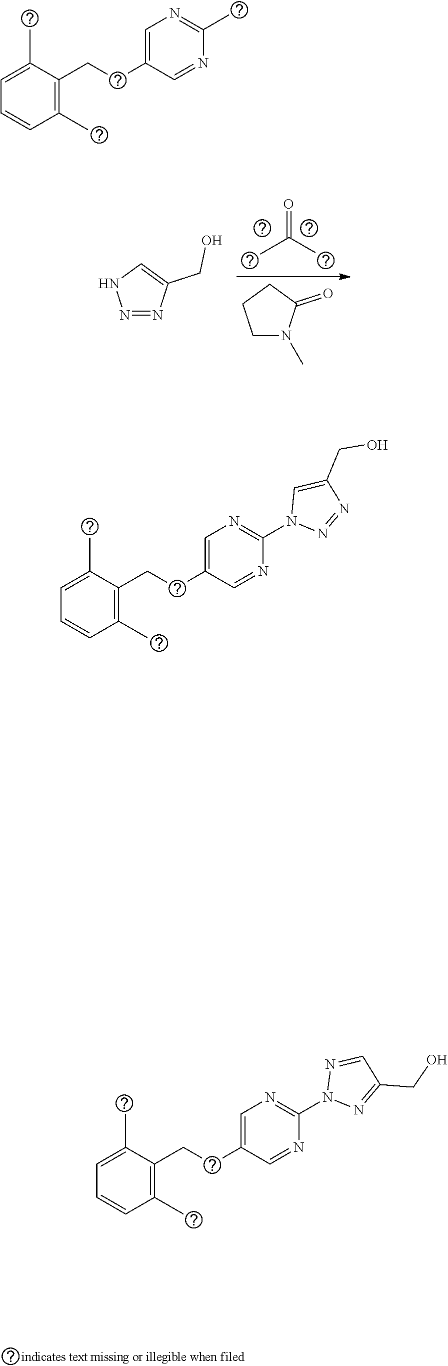

A solution of 2-chloro-5-[(2,6-dichlorophenyl) methoxy]pyrimidine, 3527-RJP-037-001 (50 mg, 0.17 mmol, 1 eq), 1H-1,2,3-triazol-4-ylmethanol (17.11 mg, 0.17 mmol, 1 eq) and POTASSIUM CARBONATE (47.73 mg, 0.35 mmol, 2 eq) in 1-METHYL-2-PYRROLIDINONE (1 mL, 1.02 g/mL, 10.29 mmol, 59.58 eq) was heated to 80oC, where it was maintained overnight. LCMS shows main peak with correct mass for product. The reaction was poured on to water then extracted with EtOAc. The organic extract was washed with brine then dried (MgSO4) and the solvent removed in vacuo. The resultant residue was dissolved in DMSO then filtered and purified by preparative HPLC automated chromatography (ISCO ACCQ HP150, Prep HPLC Column: Gemini-NX C18 Dimensions: 21 mm×150 mm 5 μM; Sample: 1.40 ml from tube 1) eluting with 5 to 95% ACN/0.1% FA in Water/0.1% FA Fractions 15 and 16 both gave the correct mass but with different retention times by LCMS (0.908 min for 15 (shoulder to peak) and 0.926 for fraction 16). The samples were freeze dried separately. Fraction 15 (6.36 mg). gave (2-{5-[(2,6-dichlorophenyl) methoxy]pyrimidin-2-yl}-1,2,3-triazol-4-yl) methanol (6.36 mg, 0.02 mmol, 10.46%) as a white solid (Purity: >95%). Fraction 16 (6.08 mg) gave (1-{5-[(2,6 dichlorophenyl) methoxy]pyrimidin-2-yl}-1,2,3-triazol-4-yl) methanol (6.08 mg, 0.02 mmol, 10%) as a white solid (Purity: >95%).

### Example 88

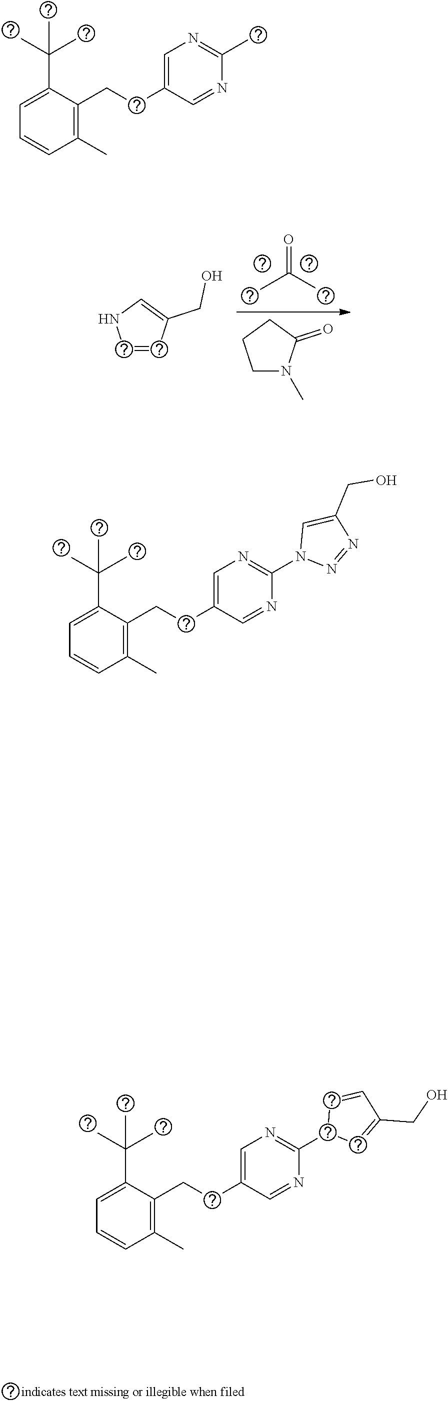

A solution of 2-chloro-5-{[2-methyl-6-(trifluoromethyl)phenyl] methoxy}pyrimidine (100 mg, 0.33 mmol, 1 eq), 1H-1,2,3-triazol4-ylmethanol (32.74 mg, 0.33 mmol, 1 eq) and POTASSIUM CARBONATE (91.32 mg, 0.66 mmol, 2 eq) in 1Methyl-2-pyyrolidinone (2 mL, 1.02 g/mL, 20.58 mmol, 62.29 eq) was heated at 80° C. under a nitrogen atmosphere overnight. The reaction was cooled to rt then water added and the compounds extracted with EtOAc. The organic extract was washed with brine then dried (MgSO4) and the solvent removed in vacuo. The resultant residue was dissolved in DMSO, filtered then purified by preparative HPLC automated chromatography (ISCO ACCQ HP150, Prep HPLC Column: Big Gemini-NX C18 Dimensions: 30 mm×250 mm 5 μM; Sample: 1.50 ml from tube 1) eluting with 5 to 95% ACN/0.1% FA in Water/0.1% FA. Fractions 45-47 were combined then freeze dried giving [1-(5-{[2-methyl-6-(trifluoromethyl)phenyl] methoxy}pyrimidin-2-yl)-1,2,3-triazol-4-yl]methanol (11.81 mg, 0.03 mmol, 9.79%) as a white solid (Purity: >95%).

### Example 89

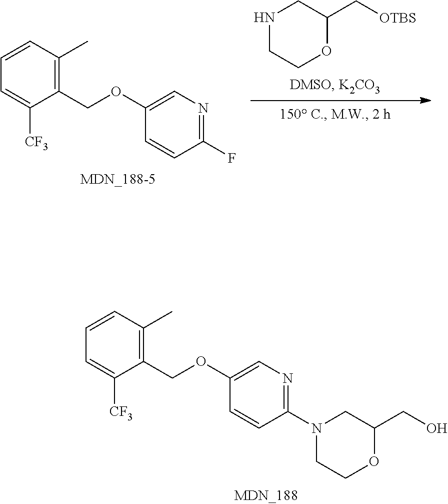 [SMILES: Cc1cccc(C(F)(F)F)c1COc1ccc(F)nc1 ; CC(C)(C)[Si](C)(C)OCC1CNCCO1 ; Cc1cccc(C(F)(F)F)c1COc1ccc(N2CCOC(CO)C2)nc1]

A mixture of MDN_188-5 (100 mg, 0.351 mmol), 2-(((tert-butyldimethylsilyl)oxy) methyl) morpholine (324 mg, 1.40 mmol) and K₂CO₃ (146 mg, 1.05 mmol) in DMSO (2 mL) was sealed under Ar atmosphere. The reaction was stirred at 150oC in a microwave reactor for 2 h. The reaction mixture was diluted with H₂O (30 mL) and extracted with EtOAc (30 mL×3). The organic layer was washed with brine (50 mL), dried over Na2SO4, filtered and concentrated. The residue was purified by TLC (PE: EtOAc=1:1) to afford MDN_188 as a white solid (9 mg, 6.7% yield).

### Example 90

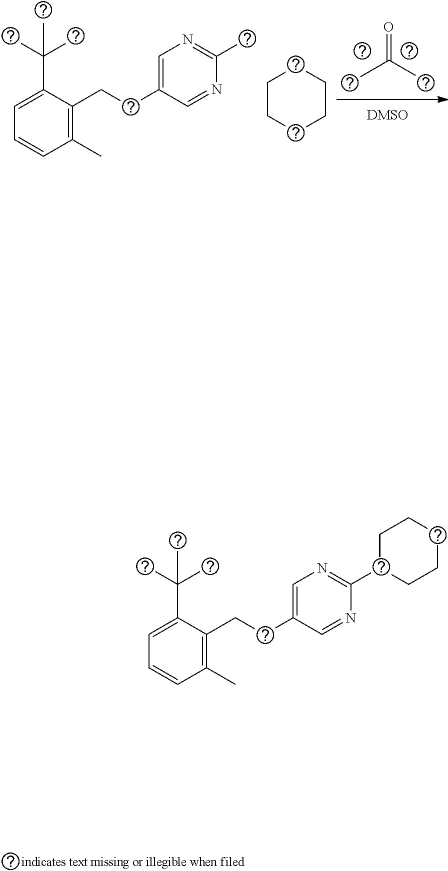

Nitrogen was bubbled through a DMSO (1 mL) solution of 2-chloro-5-{[2-methyl-6-(trifluoromethyl)phenyl] methoxy}pyrimidine (50 mg, 0.17 mmol, 1 eq), PIPERAZINE (21.34 mg, 0.25 mmol, 1.5 eq) and POTASSIUM CARBONATE (68.49 mg, 0.5 mmol, 3 eq) in a microwave vial. The vial was capped then heated to 100oC, where it was maintained for ca 2.5 h. LCMS shows the reaction has gone to completion. The reaction was cooled to rt then diluted with EtOAc and washed with water followed by brine. The organic extract was dried (MgSO4) then the solvent removed in vacuo. The resultant residue was purified by automated flash chromatography (Combiflash Rf, Silica 4 g RediSep column) eluting with 0 to 15% Methanol in Dichloromethane. Fractions 19-27 were combined then the solvent removed in vacuo. Gave 5-{[2-methyl-6-(trifluoromethyl)phenyl] methoxy}-2-(piperazin-1-yl)pyrimidine (36.7 mg, 0.1 mmol, 63.05%) as a yellow gum (Purity: >95%).

### Example 91

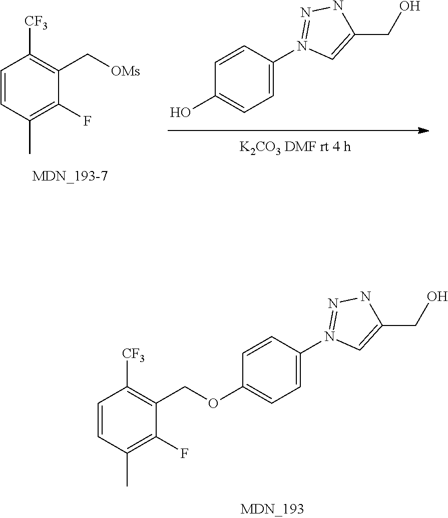

A mixture of MDN_193-7 (50 mg, 0.174 mmol), 4-(4-(hydroxymethyl)-1H-1,2,3-triazol-1-yl) phenol (34 mg, 0.174 mmol) and K₂CO₃ (36 mg, 0.261 mmol) in DMF (1.0 mL) was stirred at rt for 4 h. The mixture was quenched with water, extracted with DCM, dried over Na2SO4, purified by reversed phase column (H₂O: MeCN) to give the title compound MDN_193 as a white solid (49 mg, 74% yield).

### Example 92

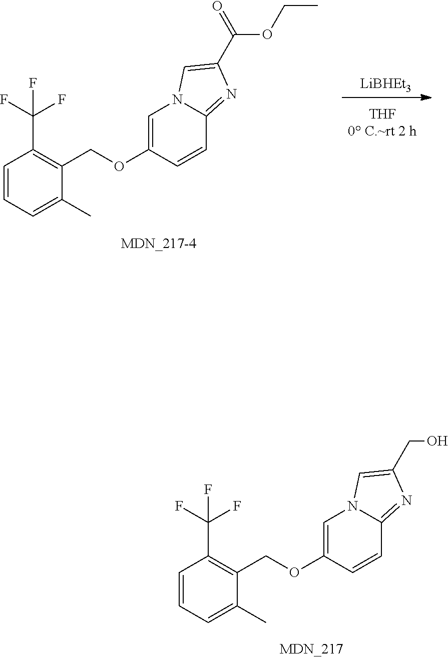 [SMILES: CCOC(=O)c1cn2cc(OCc3c(C)cccc3C(F)(F)F)ccc2n1 ; Cc1cccc(C(F)(F)F)c1COc1ccc2nc(CO)cn2c1]

To a solution of MDN_217-4 (150 mg, 0.390 mmol) in THF (1 mL) was added LiBHEt3 (1M, 1.18 mL, 1.18 mmol) at 0° C. The reaction was stirred at rt for 2 h. The reaction mixture was diluted with H₂O and extracted with EA. The organic layer was dried over Na2SO4, filtered and concentrated. The residue was purified by prep-HPLC (TFA) to obtain MDN_217 (15 mg 11.4% yield, as a white solid).

### Example 93

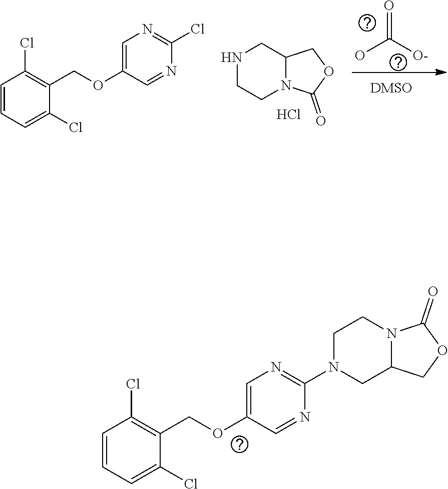

Nitrogen was bubbled through a DMSO (1.5 mL) solution of 2-chloro-5-[(2,6-dichlorophenyl) methoxy]pyrimidine (75 mg, 0.26 mmol, 1 eq), hexahydro-1H-[1,3]oxazolo [3,4-a]piperazin-3-one hydrochloride (69.4 mg, 0.39 mmol, 1.5 eq) and POTASSIUM CARBONATE (107.4 mg, 0.78 mmol, 3 eq) in a microwave vial. The vial was capped then heated to 100° C., where it was maintained overnight. The reaction was cooled to rt then diluted with EtOAc and washed with water followed by brine. The organic extract was dried (MgSO4) then the solvent removed in vacuo. The resultant residue was dissolved in DMSO then filtered and purified by preparative HPLC automated chromatography (ISCO ACCQ HP150, Prep HPLC Column: Big Gemini-NX C18 Dimensions: 30 mm×250 mm 5 μM; Sample: 1.60 ml from tube 1) eluting with 5 to 95% ACN/0.1% FA in Water/0.1% FA. Fractions 45-47 were combined then freeze dried. Gave 3527-RJP-168-001 (34 mg). Due to the purity being below 95% the sample was further purified. The compound was dissolved in DMSO then purified by preparative HPLC automated chromatography (ISCO ACCQ HP150, Sample: 1.30 ml from tube 1; Prep HPLC Column: Big Gemini-NX C18 Dimensions: 30 mm×250 mm 5 μM) eluting with 5 to 95% ACN/0.1% FA in Water/0.1% FA. Fractions 12-13 were combined then freeze dried. Gave 5-{5-[(2,6-dichlorophenyl) methoxy]pyrimidin-2-yl}-tetrahydro-3H-[1,3]oxazolo [3,4-a]piperazin-1-one (17.62 mg, 0.04 mmol, 17.21%) as a white solid (Purity: >95%).

### Example 94

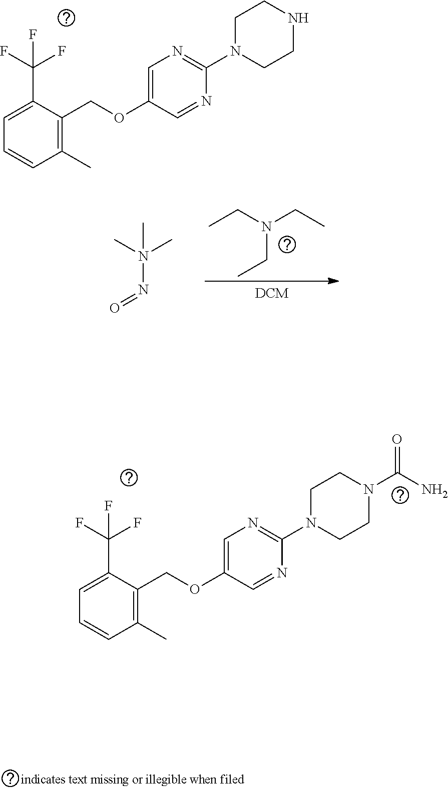

Trimethylsilyl isocyanate, 94% (0.02 mL, 0.85 g/mL, 0.15 mmol, 1.5 eq) and TRIETHYLAMINE (0.04 mL, 0.73 g/mL, 0.31 mmol, 3 eq) were added to a stirred solution of 5-{[2-methyl-6-(trifluoromethyl)phenyl] methoxy}-2-(piperazin-1-yl)pyrimidine (36 mg, 0.1 mmol, 1 eq) in DCM (2 mL). The reaction was stirred at rt overnight. LCMS shows the reaction has gone to completion. The solvent was removed in vacuo then the resultant residue dissolved in DMSO, filtered and purified by preparative HPLC automated chromatography (ISCO ACCQ HP150, Prep HPLC Column: Big Gemini-NX C18 Dimensions: 30 mm×250 mm 5 μM; Sample: 1.60 ml from tube 1) eluting with 5 to 95% ACN/0.1% FA in Water/0.1% FA. Fractions 14-17 were combined then freeze dried. Gave 4-(5-{[2-methyl-6-(trifluoromethyl)phenyl] methoxy}pyrimidin-2-yl) piperazine-1-carboxamide (23.72 mg, 0.06 mmol, 58.72%) as a white solid (Purity: >95%).

### Example 95

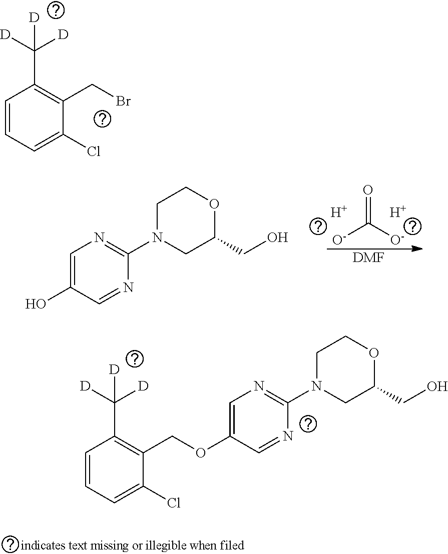

2-METHYL-6-TRIFLUOROMETHYLBENZYL BROMIDE, (57.51 mg, 0.23 mmol, 1 eq), 2-[(2S)-2-(hydroxymethyl) morpholin-4-yl]pyrimidin-5-ol (48 mg, 0.23 mmol, 1 eq) and POTASSIUM CARBONATE (157.04 mg, 1.14 mmol, 5 eq) in DMF (2 mL) was stirred at rt overnight. The reaction was diluted with EtOAc then washed with water followed by brine. The organic extract was dried (MgSO4) then the solvent removed in vacuo. The resultant residue was dissolved in DMSO then filtered and purified by preparative HPLC automated chromatography (ISCO ACCQ HP150, Prep HPLC Column: Big Gemini-NX C18 Dimensions: 30 mm×250 mm 5 μM; Sample: 1.60 ml from tube 1) eluting with 5 to 95% ACN/0.1% FA in Water/0.1% FA. Fractions 24-28 were combined then the MeCN removed in vacuo. The aq was extracted with EtOAc (×2) then the organic extract was washed with brine, dried (MgSO4) and the solvent removed in vacuo. The resultant residue was transferred to a submission vial using DCM then the solvent removed under a stream of nitrogen. The sample was dried under vacuum at 40° C. overnight. Gave [(2S)-4-(5-{[2-methyl-6-(trifluoromethyl)phenyl] methoxy}pyrimidin-2-yl) morpholin-2-yl]methanol (41.55 mg, 0.11 mmol, 47.69%) as a colourless gum (Purity: >95%).

### Example 96

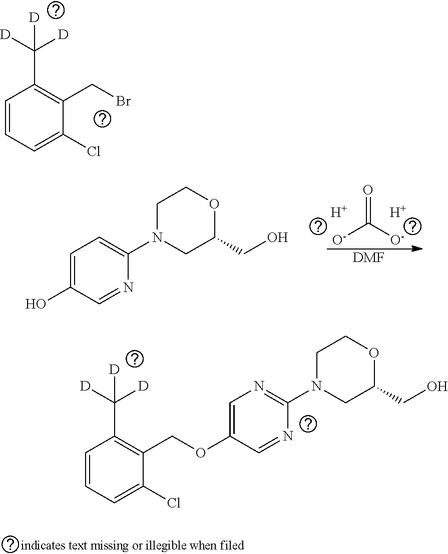

2-Chloro-6-(trifluoromethyl)benzyl bromide (62.15 mg, 0.23 mmol, 1 eq), 2-[(2S)-2-(hydroxymethyl) morpholin-4-yl]pyrimidin-5-ol (48 mg, 0.23 mmol, 1 eq) and POTASSIUM CARBONATE (157.04 mg, 1.14 mmol, 5 eq) in DMF (2 mL) was stirred at rt overnight. The reaction was diluted with EtOAc then washed with water followed by brine. The organic extract was dried (MgSO4) then the solvent removed in vacuo. The resultant residue was dissolved in DMSO then filtered and purified by preparative HPLC automated chromatography (ISCO ACCQ HP150, Sample: 1.60 ml from tube 1; Prep HPLC Column: Big Gemini-NX C18 Dimensions: 30 mm×250 mm 5 M) eluting with 5 to 95% ACN/0.1% FA in Water/0.1% FA. Fractions 20-25 were combined then the MeCN removed in vacuo. The aq was extracted with EtOAc (×2) then the organic extract washed with brine, dried (MgSO4) and the solvent removed in vacuo. The resultant residue was transferred to a vial using DCM. The solvent was removed under a stream of nitrogen then the sample dried under vacuum at 40° C. overnight. Gave [(2S)-4-(5-{[2-chloro-6-(trifluoromethyl)phenyl] methoxy}pyrimidin-2-yl) morpholin-2-yl]methanol (59.26 mg, 0.15 mmol, 64.58%) as a colourless gum (Purity: >95%).

### Example 97

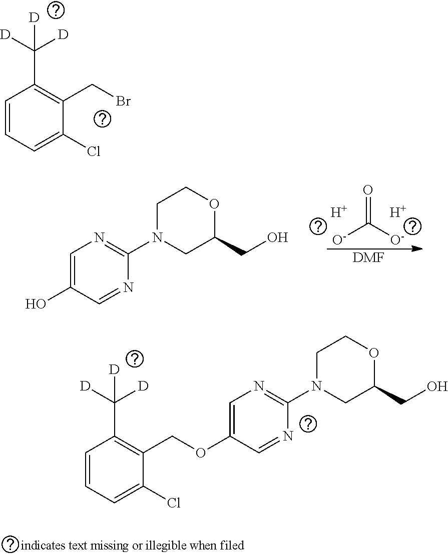

2-METHYL-6-TRIFLUOROMETHYLBENZYL BROMIDE, (46.73 mg, 0.18 mmol, 1 eq), 2-[(2R)-2-(hydroxymethyl) morpholin-4-yl]pyrimidin-5-ol, (39 mg, 0.18 mmol, 1 eq) and POTASSIUM CARBONATE (127.59 mg, 0.92 mmol, 5 eq) in DMF (2 mL) was stirred at rt overnight. The reaction was diluted with EtOAc then washed with water followed by brine. The organic extract was dried (MgSO4) then the solvent removed in vacuo. The resultant residue was dissolved in DMSO, filtered and then purified by preparative HPLC automated chromatography (ISCO ACCQ HP150, Prep HPLC Column: Big Gemini-NX C18 Dimensions: 30 mm×250 mm 5 μM; Sample: 1.60 ml from tube 1) eluting with 5 to 95% ACN/0.1% FA in Water/0.1% FA. Fractions 30-33 were combined then the MeCN removed in vacuo. The aq was extracted with EtOAc (×2) then the organic extracts washed with brine, dried (MgSO4) and the solvent removed in vacuo. The resultant residue was transferred to a vial using DCM then the solvent removed under a stream of nitrogen. The sample was dried under at 40 vacuum C overnight. Gave [(2R)-4-(5-{[2-methyl-6-(trifluoromethyl)phenyl] methoxy}pyrimidin-2-yl) morpholin-2-yl]methanol (30.52 mg, 0.08 mmol, 43.12%) as a colourless gum (Purity: >95%).

### Example 98

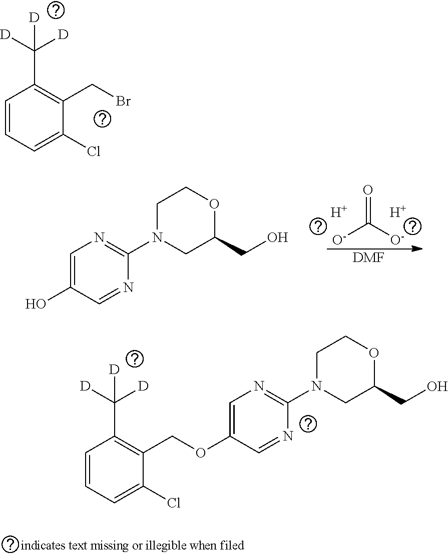

2-Chloro-6-(trifluoromethyl)benzyl bromide (50.5 mg, 0.18 mmol, 1 eq), 2-[(2R)-2-(hydroxymethyl) morpholin-4-yl]pyrimidin-5-ol (39 mg, 0.18 mmol, 1 eq) and POTASSIUM CARBONATE (127.59 mg, 0.92 mmol, 5 eq) in DMF (2 mL) was stirred at rt overnight. The reaction was diluted with EtOAc then washed with water followed by brine. The organic extract was dried (MgSO4) then the solvent removed in vacuo. The resultant residue was dissolved in DMSO, filtered and then purified by preparative HPLC automated chromatography (ISCO ACCQ HP150, Sample: 1.60 ml from tube 1; Prep HPLC Column: Big Gemini-NX C18 Dimensions: 30 mm×250 mm 5 μM) eluting with 5 to 95% ACN/0.1% FA in Water/0.1% FA Fractions 25-29 were combined then the MeCN removed in vacuo. The aq was extracted with EtOAc (×2) then the organic extracts washed with brine, dried (MgSO4) and the solvent removed in vacuo. The resultant residue was transferred to a submission vial using DCM then the solvent removed under a stream of nitrogen. The sample was dried under vacuum at 40° C. overnight. Gave [(2R)-4-(5-{[2-chloro-6-(trifluoromethyl)phenyl] methoxy}pyrimidin-2-yl) morpholin-2-yl]methanol (48.32 mg, 0.12 mmol, 64.81%) as a colourless gum (Purity: >95%) (DCM, 0.1 eq).

### Example 99

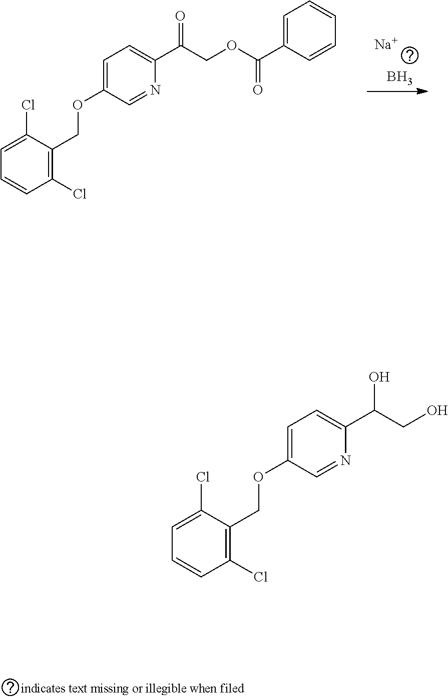

To a stirred solution of 2-{5-[(2,6-dichlorophenyl) methoxy]pyridin-2-yl}-2-oxoethyl benzoate (47.8 mg, 0.11 mmol, 1 eq) in Methanol (3 mL) was added SODIUM BOROHYDRIDE (8.69 mg, 0.23 mmol, 2 eq). Reaction stirred for 2 h at rt. LCMS showed some progression to product but still SM remaining. Reaction continued stirring for 18 h. LCMS showed complete reduction but still some ester remaining. 2 M NaOH (2 mL) was added and the mixture stirred for 10 mins. LCMS showed excellent conversion to desired product. Reaction was diluted with water (20 mL) before being extracted with ethyl acetate (3×20 mL). Combined organics were dried (phase sep paper) and concentrated to dryness affording a colourless film. The film was purified by preparative HPLC automated flash chromatography (ISCO ACCQ HP150, Prep HPLC Column: Gemini-NX C18 Dimensions: 21 mm×150 mm 5 μM; Sample: 2.50 ml from tube 1 RediSep column) eluting with 5 to 95% ACN/0.1% FA in Water/0.1% FA Dimensions: 21 mm×150 mm 5 μM; Sample: 2.50 ml from tube 1 RediSep column) eluting with 5 to 95% ACN/0.1% FA in Water/0.1% FA. Desired fractions were combined and concentrated to dryness affording 1-{5-[(2,6-dichlorophenyl) methoxy]pyridin-2-yl}ethane-1,2-diol (14.17 mg, 0.05 mmol, 39.28%) as a white powder (Purity: >95%).

### Example 100

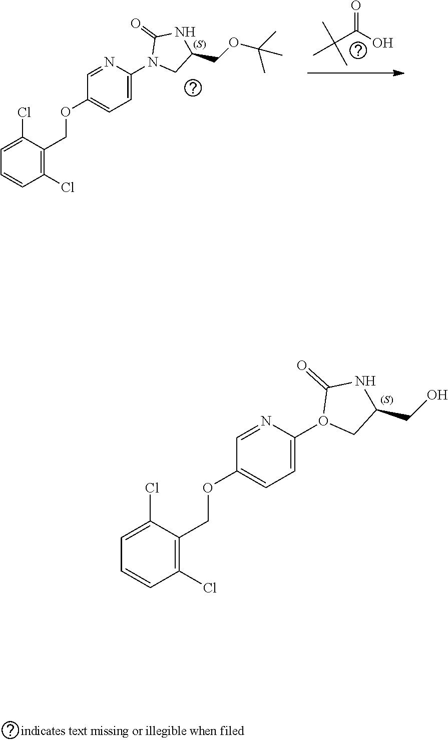

To a stirred solution of (4R)-4-(tert-butoxymethyl)-1-{5-[(2,6-dichlorophenyl) methoxy]pyridin-2-yl}imidazolidin-2-one (40.5 mg, 0.1 mmol, 1 eq) in DCM (2 mL) was added TRIFLUOROACETIC ACID (213.4 uL, 1.53 g/mL, 2.86 mmol, 30 eq). Reaction stirred at rt for 96 h at rt. Reaction was concentrated to dryness affording a colourless film. The film was purified using the Waters Prep (7 min acidic, medium mass trigger). Desired fractions were combined and concentrated to dryness affording (4R)-1-{5-[(2,6-dichlorophenyl) methoxy]pyridin-2-yl}-4-(hydroxymethyl) imidazolidin-2-one (19.34 mg, 0.05 mmol, 55.03%) as a white powder (Purity: >95%)

### Example 101

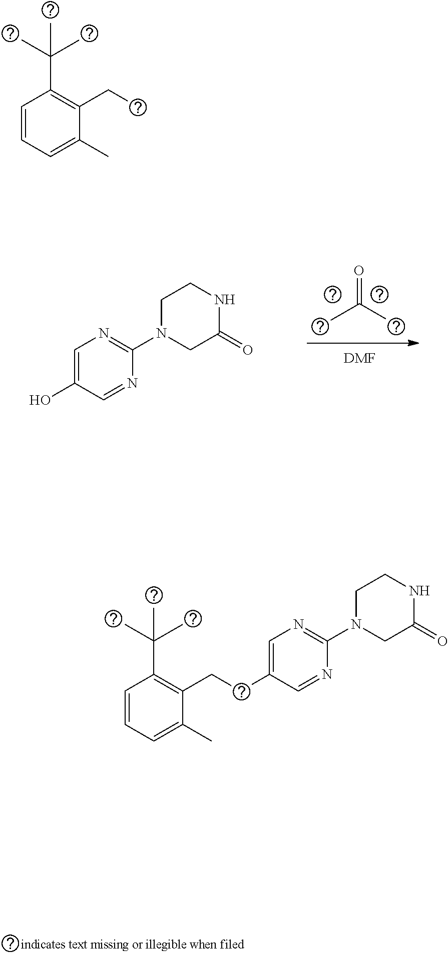

A solution of 2-METHYL-6-TRIFLUOROMETHYLBENZYL BROMIDE (39.09 mg, 0.15 mmol, 1 eq), 4-(5-hydroxypyrimidin-2-yl) piperazin-2-one (30 mg, 0.15 mmol, 1 eq) and POTASSIUM CARBONATE (106.75 mg, 0.77 mmol, 5 eq) in DMF (2 mL) was stirred at rt overnight. LCMS shows the reaction has gone to completion. The reaction was diluted with EtOAc then washed with water followed by brine. The organic extract was dried (MgSO4) then the solvent removed in vacuo. The resultant residue was dissolved in DMSO, filtered then purified by preparative HPLC automated chromatography (ISCO ACCQ HP150, Prep HPLC Column: Big Gemini-NX C18 Dimensions: 30 mm×250 mm 5 μM; Sample: 1.40 ml from tube 1) eluting with 5 to 95% ACN/0.1% FA in Water/0.1% FA Fractions 28-30 were combined then freeze dried. Gave 4-(5-{[2-methyl-6-(trifluoromethyl)phenyl] methoxy}pyrimidin-2-yl) piperazin-2-one (12.21 mg, 0.03 mmol, 21.57%) as a beige solid (Purity: >95%).

### Example 102

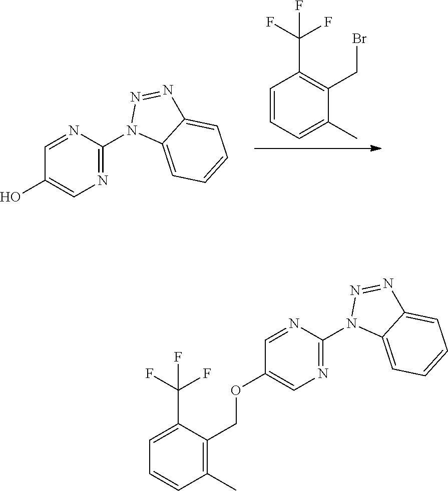 [SMILES: Oc1cnc(-n2nnc3ccccc32)nc1 ; Cc1cccc(C(F)(F)F)c1CBr ; Cc1cccc(C(F)(F)F)c1COc1cnc(-n2nnc3ccccc32)nc1]

To a solution of the starting compound above (6.4 mg, 0.03 mmol, 1 eq) in DMF (5 mL) was added 2-METHYL-6-TRIFLUOROMETHYLBENZYL BROMIDE, (7.59 mg, 0.03 mmol, 1 eq) cesium carbonate (39.1 mg, 0.12 mmol, 4 eq). Stirred at rt overnight. The reaction mixture was diluted with EtOAc (20 mL) and water (20 mL). The organic layer was separated, washed with brine, dried over MgSO4 and conc in vacuum. purified by preparative HPLC automated chromatography (ISCO ACCQ HP150, Prep HPLC Column: Gemini-NX C18 Dimensions: 21 mm×150 mm 5 μM; Sample: 2.80 ml from tube 1) eluting with 20 to 95% ACN/0.1% FA in Water/0.1% FA. fractions 17 to give: 1-(5-{[2-methyl-6-(trifluoromethyl)phenyl] methoxy}pyrimidin-2-yl)-1,2,3-benzotriazole (3.71 mg, 0.01 mmol, 32.09%) white solid (Purity: >95%)

### Example 103

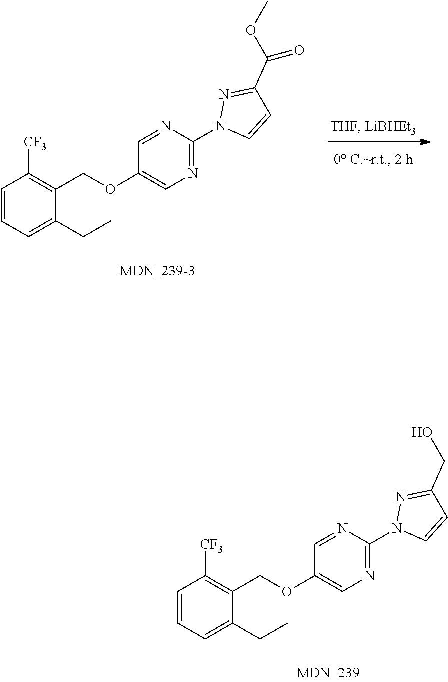 [SMILES: CCc1cccc(C(F)(F)F)c1COc1cnc(-n2ccc(C(=O)OC)n2)nc1 ; CCc1cccc(C(F)(F)F)c1COc1cnc(-n2ccc(CO)n2)nc1]

To a solution of MDN_239-3 (47 mg, 0.116 mmol) in anhydrous THF (1 mL) at 0° C. was added LiBHEt3 (0.6 mL, 0.578 mmol) slowly. The reaction was stirred at r.t. for 2 h. The reaction mixture was quenched with H₂O (10 mL) and extracted with EtOAc (10 mL×3). The organic layer was dried over Na2SO4, filtered and concentrated in vacuo. The residue was purified by TLC (MeOH:DCM=20:1) to afford MDN_239 as a white solid (20 mg, 45.6% yield).

### Example 104

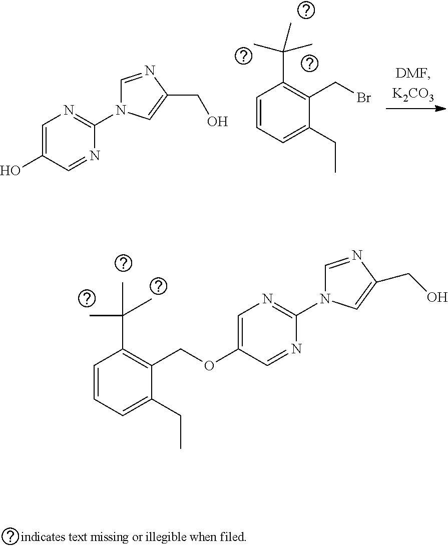 [SMILES: OCc1cn(-c2ncc(O)cn2)cn1 ; CCc1cccc(C(C)(C)C)c1CBr ; CCc1cccc(C(C)(C)C)c1COc1cnc(-n2cnc(CO)c2)nc1]

2-[4-(hydroxymethyl) imidazol-1-yl]pyrimidin-5-ol (108 mg, 0.56 mmol, 1 eq) was taken up in DMF (3 mL) and 2-(bromomethyl)-1-ethyl-3-(trifluoromethyl)benzene (165.11 mg, 0.62 mmol, 1.1 eq) was added, followed by potassium carbonate (388.35 mg, 2.81 mmol, 5 eq). Rxn was stirred at rt under N₂. Once reaction was complete by LC-MS, it was concentrated in vacuo. The residue was partitioned between EtOAc x2/H2O. The combined organics were dried over MgSO4, filtered and conc in vacuo. The residue was loaded onto isolute. purified by automated flash chromatography (Combiflash 300+, Silica 12 g RediSep column) eluting with 0 to 12% Methanol in Dichloromethane Just co-elution observed. all fractions combined and conc in vacuo, then taken up in 2 ml DMSO and filtered. purified by preparative HPLC automated chromatography (ISCO ACCQ HP150, Prep HPLC Column: Big Gemini-NX C18 Dimensions: 30 mm×250 mm 5 μM; Sample: 2.50 ml from tube 1) eluting with 5 to 95% ACN/0.1% FA in Water/0.1% FA F34 to F44 combined and conc in vacuo. T=38.578 g residue was triturated from ether, filtered and dried under suction-solid was transfured to a vial and dried in vacuo at 40° C. overnight to give [1-(5-{[2-ethyl-6-(trifluoromethyl)phenyl] methoxy}pyrimidin-2-yl) imidazol-4-yl]methanol (53.27 mg, 0.14 mmol, 25.05%) as a white solid.

### Example 105

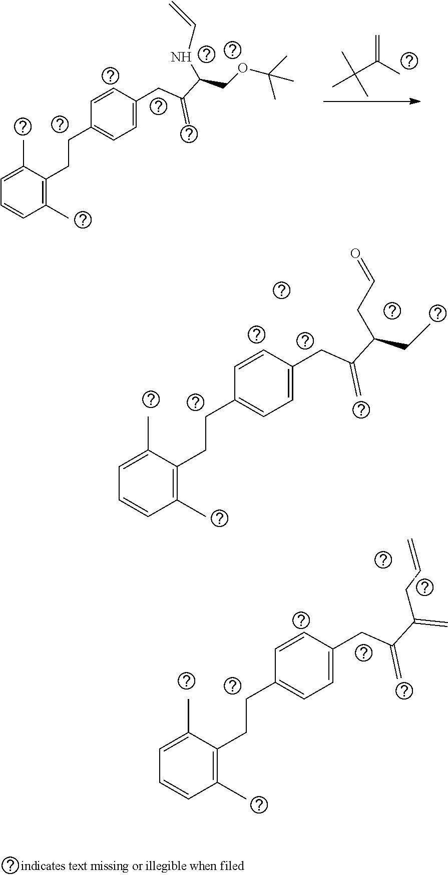

To a stirred solution of (2S)-3-(tert-butoxy)-N-{5-[(2,6-dichlorophenyl) methoxy]pyridin-2-yl}-2-formamidopropanamide, (35.6 mg, 0.08 mmol, 1 eq) in DCM (2 mL) was added TRIFLUOROACETIC ACID, (60.25 L, 1.53 g/mL, 0.81 mmol, 10 eq). Reaction stirred for 1 h at rt. Reaction was concentrated to dryness before being purified by preparative HPLC automated flash chromatography (ISCO ACCQ HP150, Sample: 2.50 ml from tube 2; Prep HPLC Column: Gemini-NX C18 Dimensions: 21 mm×150 mm 5 μM Redi Sep column) eluting with 5 to 95% ACN/0.1% FA in Water/0.1% FA Desired fractions were combined and concentrated to dryness affording (2S)-N-{5-[(2,6-dichlorophenyl) methoxy]pyridin-2-yl}-3-hydroxy-2-formamidopropanamide (18.4 mg, 0.05 mmol, 59.23%) as a white powder (Purity: 90-95%)

### Example 106

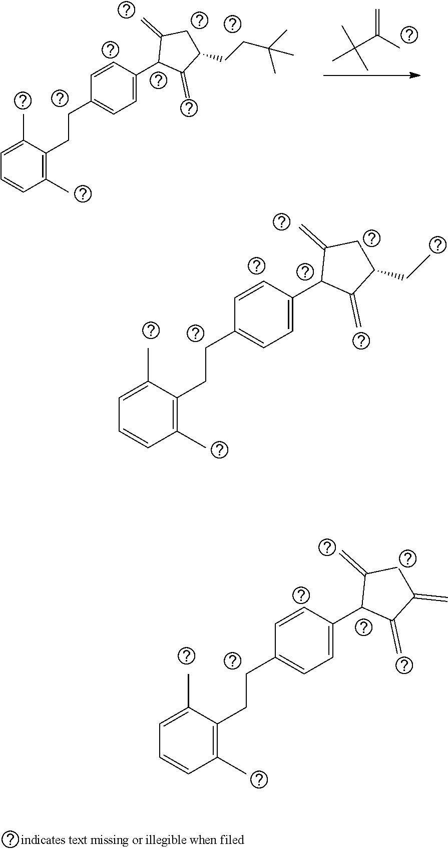

To a stirred solution of (5R)-5-(tert-butoxymethyl)-3-{5-[(2,6-dichlorophenyl) methoxy]pyridin-2-yl}imidazolidine-2,4-dione (85.7 mg, 0.2 mmol, 1 eq) in DCM (3 mL) was added TRIFLUOROACETIC ACID (145.72 uL, 1.53 g/mL, 1.96 mmol, 10 eq). Reaction stirred for 18 h at rt. Reaction was concentrated to give a colourless oil. The oil was purified by preparative HPLC automated flash chromatography (ISCO ACCQ HP150, Prep HPLC Column: Gemini-NX C18 Dimensions: 21 mm×150 mm 5 μM; Sample: 2.50 ml from tube 1 RediSep column) eluting with 5 to 95% ACN/0.1% FA in Water/0.1% FA, Desired fractions were combined and concentrated to dryness affording (5R)-3-{5-[(2,6-dichlorophenyl) methoxy]pyridin-2-yl}-5-(hydroxymethyl) imidazolidine-2,4-dione (37.2 mg, 0.1 mmol, 49.78%) as a white powder (Purity: >95%)

### Example 108

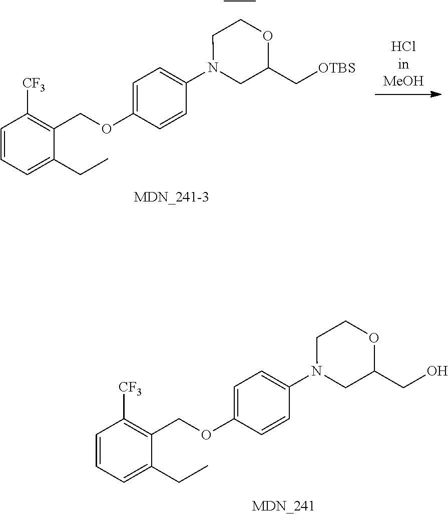

A solution of MDN_241-3 (69 mg, 1.25 mmol) in HCl in MeOH (4M 2 mL) was stirred at rt for 3 h. The mixture was concentrated, diluted with water, adjusted PH>7, extracted with DCM, dried over Na2SO4, purified by pre-TLC to give the title compound MDN_241 as a pale solid (18 mg, 34% yield).

### Example 109

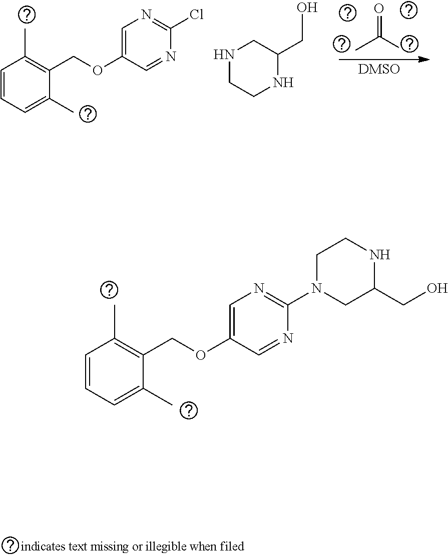

Nitrogen was bubbled through a DMSO (2 mL) solution of 2-chloro-5-[(2,6-dichlorophenyl) methoxy]pyrimidine (100 mg, 0.35 mmol, 1 eq), piperazin-2-ylmethanol, (40.12 mg, 0.35 mmol, 1 eq) and POTASSIUM CARBONATE (143.2 mg, 1.04 mmol, 3 eq) then the tube sealed and heated to 100° C., where it was maintained for ca 24 h. The reaction was cooled to rt then diluted with EtOAc and washed with water followed by brine. The organic extract was dried (MgSO4) then the solvent removed in vacuo. The resultant residue was dissolved in DMSO, filtered and purified by preparative HPLC automated chromatography (ISCO ACCQ HP150, Prep HPLC Column: Big Gemini-NX C18 Dimensions: 30 mm×250 mm 5 μM; Sample: 1.60 ml from tube 1) eluting with 5 to 95% ACN/0.1% FA in Water/0.1% FA. Fractions 14-25 were combined then freeze dried then the resultant residue dissolved in DCM/MeOH and transferred to a vial. The solvent was removed under a stream of nitrogen then the sample dried overnight under vacuum at 40° C. Gave (4-{5-[(2,6-dichlorophenyl) methoxy]pyrimidin-2-yl}piperazin-2-yl) methanol (44.6 mg, 0.12 mmol, 34.97%) as a colourless gum (Purity: >95%).

### Example 110

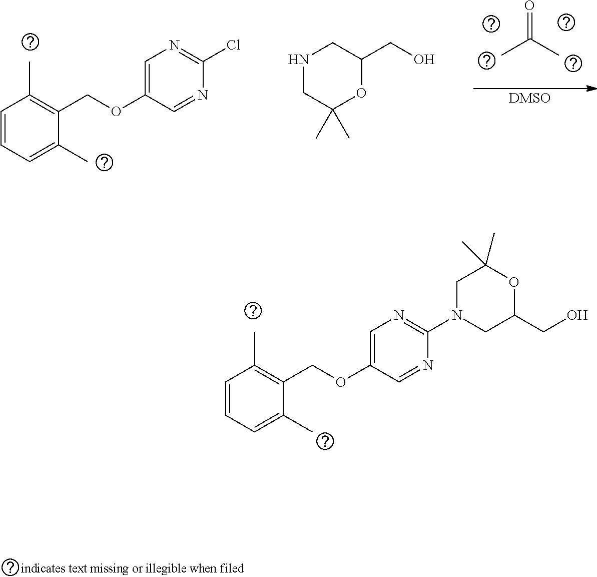

Nitrogen was bubbled through a DMSO (2 mL) solution of 2-chloro-5-[(2,6-dichlorophenyl) methoxy]pyrimidine (100 mg, 0.35 mmol, 1 eq), (6,6-dimethylmorpholin-2-yl) methanol (50.15 mg, 0.35 mmol, 1 eq) and POTASSIUM CARBONATE (143.2 mg, 1.04 mmol, 3 eq). The tube was sealed then heated to 100° C., where it was maintained for ca 24 h. The reaction was cooled to rt then diluted with EtOAc and washed with water followed by brine. The organic extract was dried (MgSO4) then the solvent removed in vacuo. The resultant residue was dissolved in DMSO then filtered and purified by preparative HPLC automated chromatography (ISCO ACCQ HP150, Sample: 1.40 ml from tube 1; Prep HPLC Column: Big Gemini-NX C18 Dimensions: 30 mm×250 mm 5 μM) eluting with 5 to 95% ACN/0.1% FA in Water/0.1% FA Fractions 37-41 were combined then freeze dried. The resultant glass was transferred to a submission vial using DCM then the solvent removed under a stream of nitrogen. The compound was dried under vacuum at 40° C. overnight. Gave (4-{5-[(2,6-dichlorophenyl) methoxy]pyrimidin-2-yl}-6,6-dimethylmorpholin-2-yl) methanol (43.86 mg, 0.11 mmol, 31.89%) as a colourless gum (Purity: 90-95%).

### Example 111

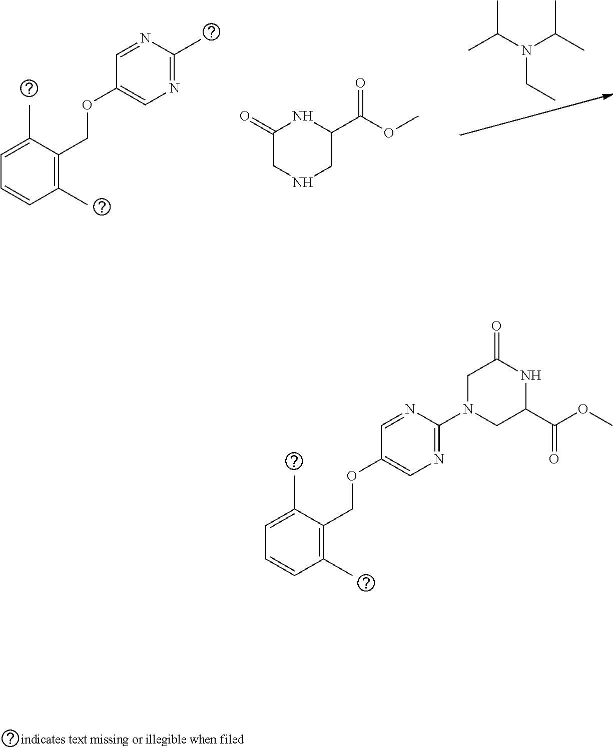

To a microwave vial containing 2-chloro-5-[(2,6-dichlorophenyl) methoxy]pyrimidine (100 mg, 0.35 mmol, 1 eq) and N,N-DIISOPROPYLETHYLAMINE (0.17 mL, 0.78 g/mL, 1.04 mmol, 3 eq) in DMSO (3 mL) was added Methyl 6-oxopiperazine-2-carboxylate (60.08 mg, 0.38 mmol, 1.1 eq). Reaction was sealed and heated to 100° C. for 18 h. The vial was subsequently irradiated to 120° C. for 1 h using a SEM reactor. The reaction mixture was directly purified using Waters prep-LCMS (Big Gem 30 min method, low mass trigger). Desired fractions were combined and concentrated to dryness affording methyl 4-{5-[(2,6-dichlorophenyl) methoxy]pyrimidin-2-yl}-6-oxopiperazine-2-carboxylate (38.6 mg, 0.09 mmol, 27.18%) as a off-white powder (Purity: >95%)

### Example 112

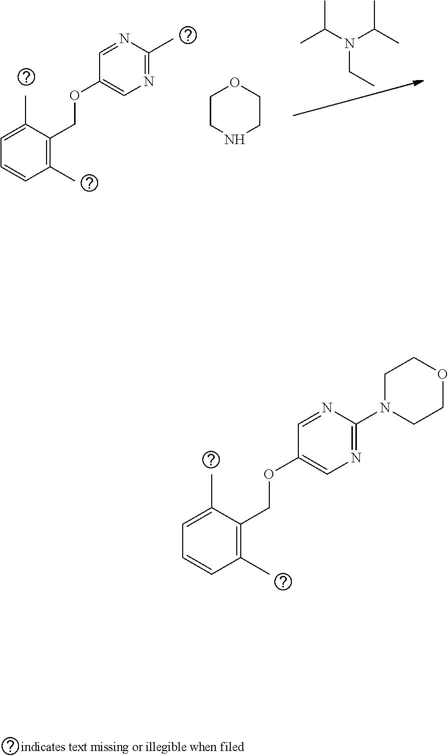

A solution of 2-chloro-5-[(2,6-dichlorophenyl) methoxy]pyrimidine (50 mg, 0.17 mmol, 1 eq) in DMSO (2 mL) with MORPHOLINE (22.79 μL, 0.99 g/mL, 0.26 mmol, 1.5 eq) and N,N-DIISOPROPYLETHYLAMINE (57.23 μL, 0.78 g/mL, 0.35 mmol, 2 eq) was irradiated to 140° C. for 15 mins. The reaction mixture was directly purified by Waters prep-LCMS (Big Gem 30 min method, low mass trigger). Fractions pertaining to product were combined and concentrated to dryness affording 4-{5-[(2,6-dichlorophenyl) methoxy]pyrimidin-2-yl}morpholine (43 mg, 0.13 mmol, 73.19%) as a white powder (Purity: >95%)

### Example 114

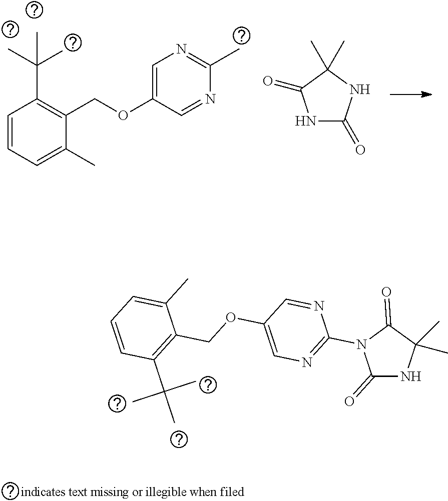

A 15 mL pressure tube was charged with the hydantoin (3.0 mmol) and copper oxide (I) (286 mg, 2.0 mmol); the aryl halide (2.0 mmol) was added at this stage if solid. The tube was fitted with a rubber septum, evacuated under high vacuum, and backfilled with argon (three times) before adding the aryl halide (added at this stage if liquid) (2.0 mmol) and anhydrous DMF (5 mL). The rubber septum was then replaced by a Teflon-coated screw cap before heating the heterogeneous reaction mixture at 150° C. for 14 h. The suspension was cooled to room temperature and filtered through a pad of Celite (washed with EtOAc), and the filtrate was concentrated to ca. one tenth of its volume under reduced pressure, poured into a mixture of ice and water (10 mL), and stirred for 30 min before adding a 28% aqueous ammonia solution (3 mL). The resulting suspension was stirred for 30 min, and the precipitate was collected by filtration and then dried under high vacuum to give the desired arylated hydantoin which was, whenever required, further purified by flash column chromatography over silica gel. 2-bromo-5-{[2-methyl-6-(trifluoromethyl)phenyl] methoxy}pyrimidine (70 mg, 0.2 mmol, 1 eq) 5,5-Dimethylhydantoin (38.76 mg, 0.3 mmol, 1.5 eq) and COPPER (I) OXIDE (28.85 mg, 0.2 mmol, 1 eq) were diluted with DMF (0.5 mL) under nitrogen and stirred in a sealed vial at 150° C. for 4 Hours. The reaction mixture was then filtered through celite, eluting with ethyl acetate (30 mL), concentrated to ˜2 mL under reduced pressure, to which was added two pieces of ice, then ammonium hydroxide (28%, 0.3 mL), extracted with ethyl acetate (2×15 mL), washed with 5% lithium chloride (2×15 mL), brine (15 mL), passed through a hydrophobic frit and concentrated under reduced pressure to give a brown gum (71 mg) which was further purified by HP. Fractions containing the desired product (19-23) were lyophilised to give a white powder which was diluted with methanol, transferred to a vial and concentrated by Biotage V10 to give 5,5-dimethyl-3-(5-{[2-methyl-6-(trifluoromethyl)phenyl] methoxy}pyrimidin-2-yl) imidazolidine-2,4-dione (37.5 mg, 0.1 mmol, 47.16%) as a white solid (Purity: >95%)

### Example 115

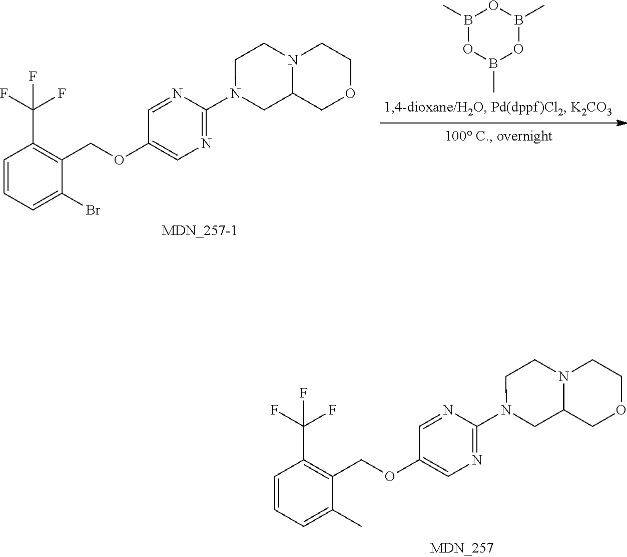

A mixture of MDN_257-1 (50 mg, 0.106 mmol), 2,4,6-trimethyl-1,3,5,2,4,6-trioxatriborinane (0.15 mL, 0.528 mmol), K₂CO₃ (44 mg, 0.318 mmol) and Pd(dppf)Cl2 (15 mg, 0.021 mmol) in 1,4-dioxane (1 mL) and H₂O (0.1 mL) was stirred at 1000° C. under Ar atmosphere overnight. The reaction mixture was diluted with H₂O (20 mL) and extracted with EtOAc (20 mL×2). The organic layer was dried over Na2SO4, filtered and concentrated in vacuo. The residue was purified by TLC (PE:EA=2:1) to afford MDN_257 as a white solid (14 mg, 32.5% yield).

### Example 116

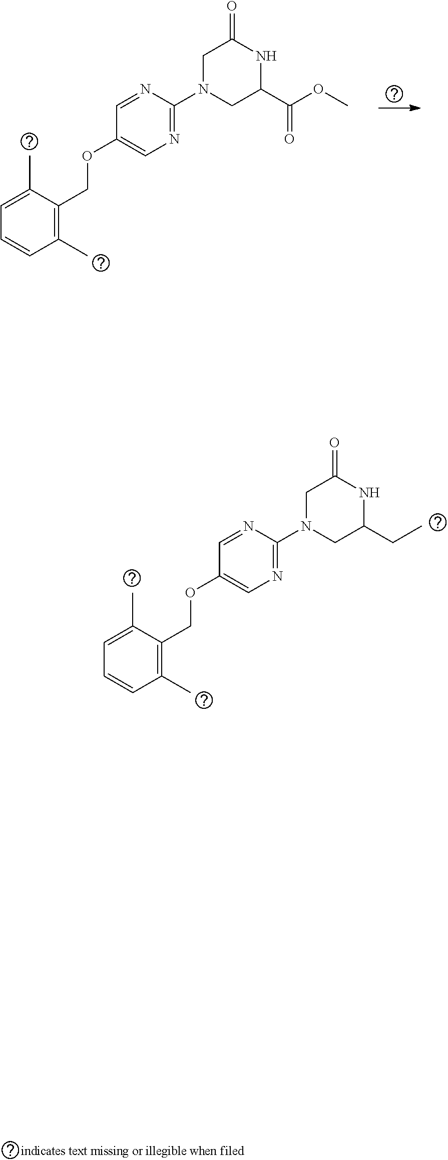 [SMILES: COC(=O)C1CN(c2ncc(OCc3c(C)cccc3C)cn2)CC(=O)N1 ; CCC1CN(c2ncc(OCc3c(C)cccc3C)cn2)CC(=O)N1]

To a stirred solution of methyl 4-{5-[(2,6-dichlorophenyl) methoxy]pyrimidin-2-yl}-6-oxopiperazine-2-carboxylate (37.5 mg, 0.09 mmol, 1 eq) in ethanol (3 mL) was added SODIUM BOROHYDRIDE (4.14 mg, 0.11 mmol, 1.2 eq). Reaction stirred for 4 h at rt. 2 M HCl (1 mL) was added to quench the reaction causing effervescence. The resulting solution was concentrated to dryness affording a white film. The film was purified by preparative HPLC automated flash chromatography (ISCO ACCQ HP150, Prep HPLC Column: Gemini-NX C18 Dimensions: 21 mm×150 mm 5 μM; Sample: 2.50 ml from tube 1 RediSep column) eluting with 5 to 95% ACN/0.1% FA in Water/0.1% FA Desired fractions were combined and concentrated to dryness affording 4-{5-[(2,6-dichlorophenyl) methoxy]pyrimidin-2-yl}-6-(hydroxymethyl) piperazin-2-one (12.5 mg, 0.03 mmol, 35.77% piperazin-2-one (12.5 mg, 0.03 mmol, 35.77%) as a as a white powder (Purity: >95%)

### Example 117

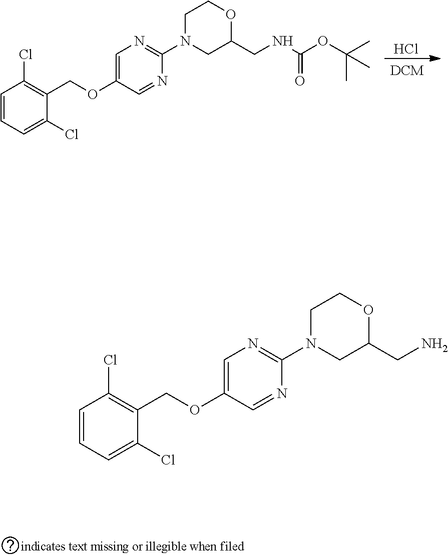 [SMILES: CC(C)(C)OC(=O)NCC1CN(c2ncc(OCc3c(Cl)cccc3Cl)cn2)CCO1 ; Cl ; NCC1CN(c2ncc(OCc3c(Cl)cccc3Cl)cn2)CCO1]

HCl solution (4N in 1,4-dioxane) (0.14 mL, 4M in, 0.56 mmol, 4 eq) was added to a stirred solution of tert-butyl N-[(4-{5-[(2,6-dichlorophenyl) methoxy]pyrimidin-2-yl}morpholin-2-yl)methyl]carbamate (66 mg, 0.14 mmol, 1 eq) in DCM (4 mL). The reaction was stirred at rt overnight. The solvent was removed in vacuo then the resultant residue dissolved in DMSO, filtered and purified by preparative HPLC automated chromatography (ISCO ACCQ HP150, Prep HPLC Column: Big Gemini-NX C18 Dimensions: 30 mm×250 mm 5 μM; Sample: 1.40 ml from tube 1) eluting with 5 to 95% ACN/0.1% FA in Water/0.1% FA chromatography (ISCO ACCQ HP150, Prep HPLC Column: Big Gemini-NX C18 Dimensions: 30 mm×250 mm 5 μM; Sample: 1.40 ml from tube 1) eluting with 5 to 95% ACN/0.1% FA in Water/0.1% FA Fractions 12-21 were combined then freeze dried. The resultant glass was transferred to a submission vial using DCM/MeOH then the solvent removed under a stream of nitrogen. The compound was dried under vacuum at 40° C. overnight. Gave (4-{5-[(2,6-dichlorophenyl) methoxy]pyrimidin-2-yl}morpholin-2-yl) methanamine (39.81 mg, 0.11 mmol, 76.67%) as a white solid (Purity: >95%) (FORMATE, 1 eq).

### Example 118

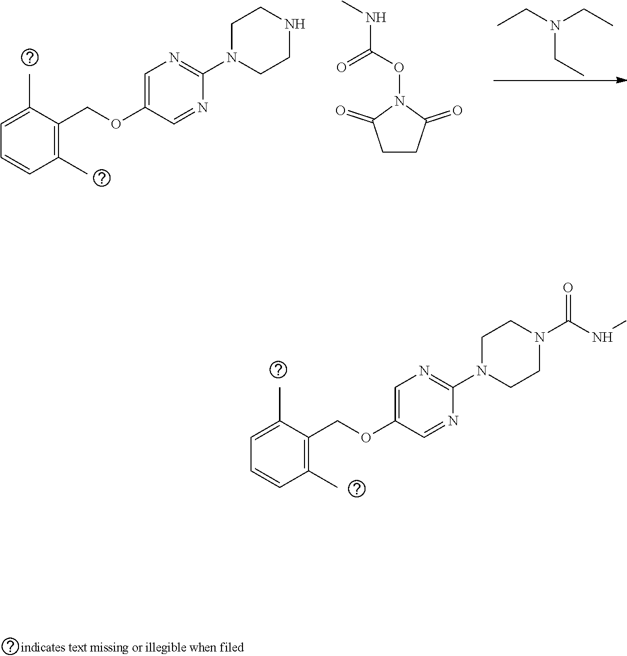

To a stirred solution of 5-[(2,6-dichlorophenyl) methoxy]-2-(piperazin-1-yl) pyrimidine (40 mg, 0.12 mmol, 1 eq) in DCM (2 mL) with TRIETHYLAMINE (0.03 mL, 0.73 g/mL, 0.24 mmol, 2 eq) was added N-Succinimidyl N-methylcarbamate, 97% (24.36 mg, 0.14 mmol, 1.2 eq). Reaction stirred for 1 h at rt. Reaction was quenched by addition of MeOH before being concentrated to dryness affording a cream film. The film was purified by preparative HPLC automated flash chromatography (ISCO ACCQ HP150, Prep HPLC Column: Gemini-NX C18 Dimensions: 21 mm×150 mm 5 μM; Sample: 2.50 ml from tube 1 RediSep column) eluting with 5 to 95% ACN/0.1% FA in Water/0.1% FA Desired fractions were combined and concentrated to dryness affording 4-{5-[(2,6-dichlorophenyl) methoxy]pyrimidin-2-yl}-N-methylpiperazine-1-carboxamide (26.12 mg, 0.07 mmol, 55.9%) as a white powder (Purity: >95%)

### Example 119

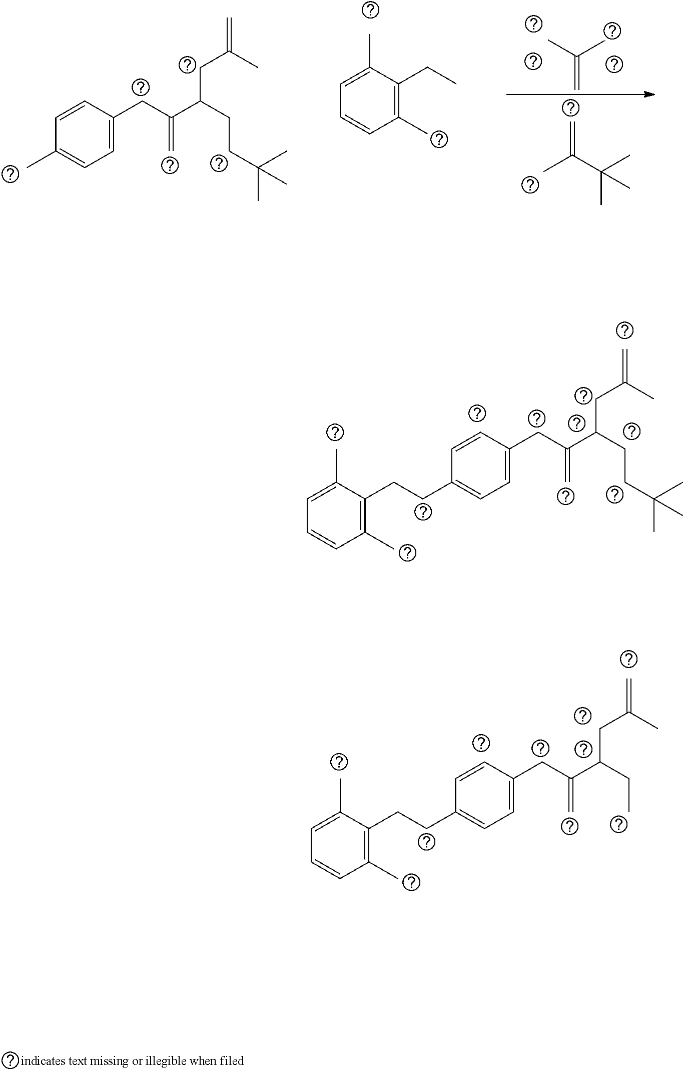

To a stirred solution of (2R)-3-(tert-butoxy)-2-acetamido-N-(5-hydroxypyridin-2-yl) propanamide (70.4 mg, 0.24 mmol, 1 eq) in DMF (2 mL) with POTASSIUM CARBONATE (41.18 mg, 0.3 mmol, 1.25 eq) was added 2,6-Dichlorobenzyl bromide (68.63 mg, 0.29 mmol, 1.2 eq). Reaction stirred for 2 h at rt. The reaction was filtered through a plug of celite before being concentrated to dryness affording a brown gum. The gum was taken into DCM (2 mL) before TRIFLUOROACETIC ACID (88.82 μL, 1.53 g/mL, 1.19 mmol, 5 eq) was added. Reaction stirred for 18 h at rt. LC-MS showed small conversion to product but mainly SM remaining. TRIFLUOROACETIC ACID, VERC100604:9 (88.82 μL, 1.53 g/mL, 1.19 mmol, 5 eq) was added and the mixture stirred for 2 h. Reaction was quenched by addition of sat sodium bicarbonate solution (10 mL). The mixture was extracted with ethyl acetate (3×15 mL). Combined organics were dried (phase separatior) and concentrated to dryness affording a colourless film. were dried (phase separatior) and concentrated to dryness affording a colourless film. The film was purified by preparative HPLC automated flash chromatography (ISCO ACCQ HP150, Prep HPLC Column: Gemini-NX C18 Dimensions: 21 mm×150 mm 5 μM; Sample: 2.50 ml from tube 1 RediSep column) eluting with 5 to 95% ACN/0.1% FA in Water/0.1% FA Desired fractions were combined and concentrated to dryness affording (2R)-N-{5-[(2,6-dichlorophenyl) methoxy]pyridin-2-yl}-2-acetamido-3-hydroxypropanamide (9 mg, 0.02 mmol, 9.48%) as a as a white powder (Purity: >95%).

### Example 120

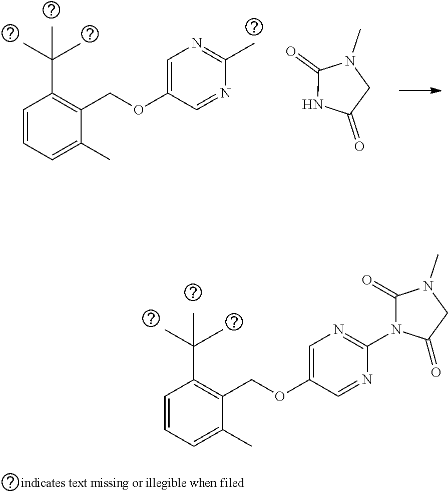

2-bromo-5-{[2-methyl-6-(trifluoromethyl)phenyl] methoxy}pyrimidine (34.7 mg, 0.1 mmol, 1 eq) 1-Methylhydantoin (28.51 mg, 0.25 mmol, 2.5 eq) and COPPER (I) OXIDE (14.3 mg, 0.1 mmol, 1 eq) under nitrogen were diluted with DMF (0.3 mL) and stirred in a sealed HPLC vial in F/C R at 150° C. for 4 Hours 10 Aug. 2021 LCMS analysis showed 100% conversion of starting material and the formation of two products. The reaction mixture was filtered through celite, eluting with ethyl acetate (40 mL), washed with a saturated aqueous solution of ammonium chloride (30 mL), 5% aqueous lithium chloride (2×20 mL), brine (20 mL), passed through hydrophobic filter paper and concentrated under reduced pressure to give a yellow oil which was diluted with DMSO and purified by preparative HPLC automated chromatography (ISCO ACCQ HP125, Prep HPLC Column: Gemini-NX C18 Dimensions: 21 mm×150 mm 5 μM; Sample: 1.60 ml from tube 1) eluting with 5 to 95% ACN/0.1% FA in Water/0.1% FA 11 Aug. 2021 Fraction 15 containing pure desired product was concentrated by Biotage V10 and dried under vacuum at 40° C. overnight to give 1-methyl-3-(5-{[2-methyl-6-(trifluoromethyl)phenyl] methoxy}pyrimidin-2-yl) imidazolidine-2,4-dione (3.6 mg, 0.01 mmol, 9.47%) as a white solid (Purity: >95%).

### Example 121

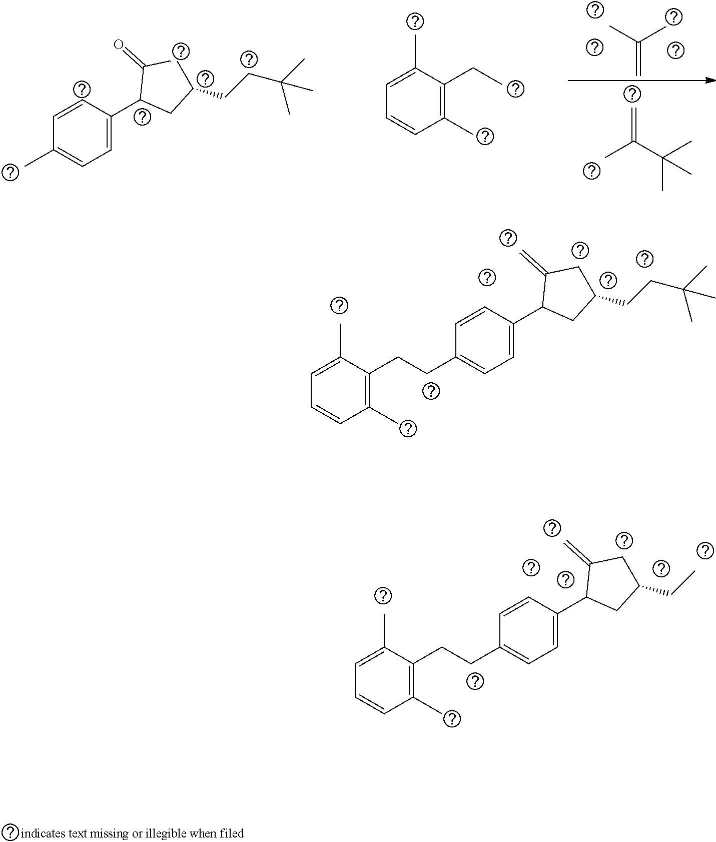

To a stirred solution solution of (4S)-4-(tert-butoxymethyl)-1-(5-hydroxypyridin-2-yl) imidazolidin-2-one (62.3 mg, 0.23 mmol, 1 eq) was added POTASSIUM CARBONATE (40.57 mg, 0.29 mmol, 1.25 eq) and 2,6-Dichlorobenzyl bromide (67.61 mg, 0.28 mmol, 1.2 eq). Reaction stirred for 1 h at rt. Reaction was filtered through a plug of celite before being concentrated to dryness affording a brown gum. The gum was taken into DCM (2 mL) and TRIFLUOROACETIC ACID (87.5 μL, 1.53 g/mL, 1.17 mmol, 5 eq) was added. Reaction stirred for 18 h at rt. LCMS showed some conversion to product but mainly SM remaining. TRIFLUOROACETIC ACID (87.5 μL, 1.53 g/mL, 1.17 mmol, 5 eq) was added and the reaction stirred for 24 h. Saturated sodium bicarb solution was added before being extracted with DCM (3×20 mL). Combined organics were dried (phase separator) andconcentrated to dryness affording a brown film. The film was purified by preparative HPLC automated flash chromatography (ISCO ACCQ HP150, Prep HPLC Column: Gemini-NX C18 Dimensions: 21 mm×150 mm 5 μM; Sample: 2.50 ml from tube 1 RediSep column) eluting with 5 to 95% ACN/0.1% FA in Water/0.1% FA Dimensions: 21 mm×150 mm 5 M; Sample: 2.50 ml from tube 1 RediSep column) eluting with 5 to 95% ACN/0.1% FA in Water/0.1% FA Desired fractions were combined and concentrated to dryness affording (4S)-1-{5-[(2,6-dichlorophenyl) methoxy]pyridin-2-yl}-4-(hydroxymethyl) imidazolidin-2-one (25.62 mg, 0.07 mmol, 29.63%) as a white powder (Purity: >95%)

### Example 122

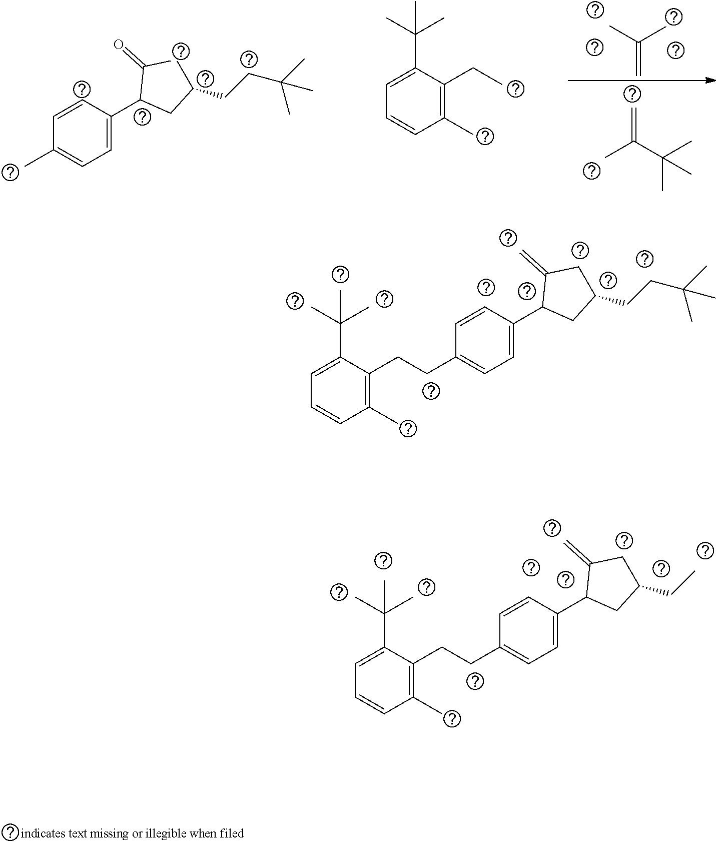

To a stirred solution of (4S)-4-(tert-butoxymethyl)-1-(5-hydroxypyridin-2-yl) imidazolidin-2-one (62.4 mg, 0.24 mmol, 1 eq) in DMF (2 mL) was added POTASSIUM CARBONATE (40.63 mg, 0.29 mmol, 1.25 eq) and 2-METHYL-6-TRIFLUOROMETHYLBENZYL BROMIDE (71.42 mg, 0.28 mmol, 1.2 eq). Reaction stirred for 2 h at rt. Reaction was filtered through a plug of celite before being concentrated to dryness affording a pink/brown gum. The gum was taken into DCM (2 mL) before TRIFLUOROACETIC ACID, VERC100604:9 (52.58 μL, 1.53 g/mL, 0.71 mmol, 3 eq) was added. Reaction stirred for 18 h at rt. LCMS showed some progression to product but still a large amount of SM remaining. TRIFLUOROACETIC ACID (52.58 μL, 1.53 g/mL, 0.71 mmol, 3 eq) was added and the reaction stirred for a further 18 h. LCMs showed good conversion to product. Reaction was concentrated to dryness affording a brown gum. The gum was purified by preparative HPLC automated flash chromatography (ISCO ACCQ HP150, Prep HPLC Column: Gemini-NX C18 Dimensions: 21 mm×150 mm 5 μM; Sample: 2.50 ml from tube 2 RediSep column) eluting with 5 to 95% ACN/0.1% FA in Water/0.1% FA Desired fractions were combined and concentrated to dryness affording (4S)-4-(hydroxymethyl)-1-(5-{[2-methyl-6-(trifluoromethyl)phenyl] methoxy}pyridin-2-yl) imidazolidin-2-one (9.58 mg, 0.03 mmol, 10.68%) as a white powder (Purity: >95%)

### Example 123

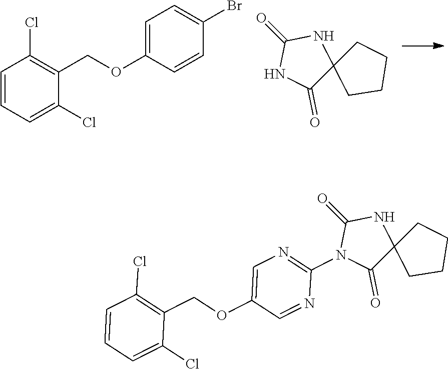

2-bromo-5-[(2,6-dichlorophenyl) methoxy]pyrimidine (46 mg, 0.14 mmol, 1 eq) 1,3-Diazaspiro[4.4]nonane-2,4-dione (31.85 mg, 0.21 mmol, 1.5 eq) and COPPER (I) OXIDE, (19.71 mg, 0.14 mmol, 1 eq) were dissolved in DMF (0.35 mL) under nitrogen and stirred in a sealed HPLC vial in F/C R at 150° C. for 4 Hours 10 Aug. 2021 LCMS analysis showed only trace of starting material. The reaction mixture was filtered through celite, eluting with ethyl acetate, washed with a saturated aqueous solution of ammonium chloride (30 mL), 5% aqueous lithium chloride (30 mL), brine (25 mL), passed through hydrophobic filter paper and concentrated under reduced pressure to give a yellow solid. The crude mixture was diluted with DMSO and purified by preparative HPLC automated chromatography (ISCO ACCQ HP125, Prep HPLC Column: Big Gemini-NX C18 Dimensions: 30 mm×250 mm 5 μM; Sample: 2.00 ml from tube 1) eluting with 5 to 95% ACN/0.1% FA in Water/0. 1% FA Fractions containing the desired product (15-17) were concentrated under reduced pressure, lyophilised, transferred to a vial with methanol, concentrated by Biotage V10 and dried under vacuum overnight to give 3-{5-[(2,6-dichlorophenyl) methoxy]pyrimidin-2-yl}-1,3-diazaspiro[4.4]nonane-2,4-dione (12.8 mg, 0.03 mmol, 22.82%) as a white powder (Purity: >95%)

### Example 124

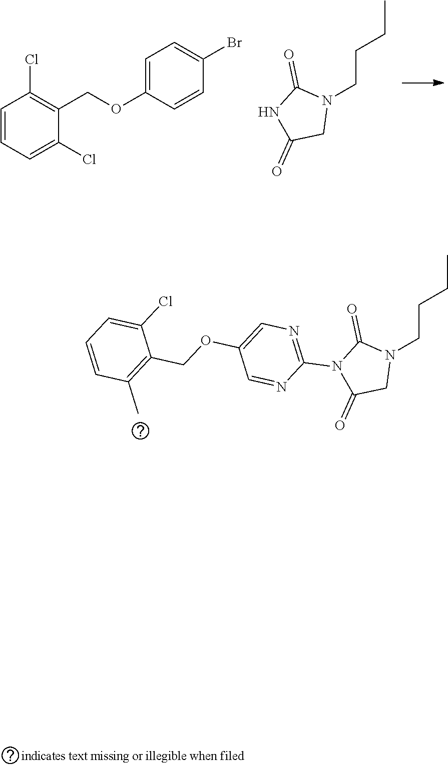 [SMILES: Clc1cccc(Cl)c1COc1ccc(Br)cc1 ; CCCCN1CC(=O)NC1=O ; CCCCN1CC(=O)N(c2ncc(OCc3c(C)cccc3Cl)cn2)C1=O]

2-bromo-5-[(2,6-dichlorophenyl) methoxy]pyrimidine (47 mg, 0.14 mmol, 1 eq) 1-Butylhydantoin (54.95 mg, 0.35 mmol, 2.5 eq) and COPPER (I) OXIDE (20.14 mg, 0.14 mmol, 1 eq) were dissolved in DMF (1 mL) under nitrogen and stirred in a sealed HPLC vial at 150° C. for 4 Hours, The reaction mixture was filtered through celite, eluting with ethyl acetate (40 mL), washed with a saturated aqueous solution of ammonium chloride (30 mL), 5% aqueous lithium chloride (25 mL), brine (25 mL), passed through hydrophobic filter paper and concentrated under reduced pressure to give a pale yellow oil. The crude mixture was diluted with DMSO and purified by preparative HPLC automated chromatography (ISCO ACCQ HP125, Prep HPLC Column: Big Gemini-NX C18 Dimensions: 30 mm×250 mm 5 μM; Sample: 2.20 ml from tube 2) eluting with 5 to 95% ACN/0.1% FA in Water/0. FA Fractions containing the desired product (22-24) were lyophilised, diluted with methanol, transferred to a vial, concentrated by Biotage V10 and dried under vacuum at 40° C. overnight to give 1-butyl-3-{5-[(2,6-dichlorophenyl) methoxy]pyrimidin-2-yl}imidazolidine-2,4-dione (10.4 mg, 0.03 mmol, 18.06%) as a white solid (Purity: >95%).

### Example 125

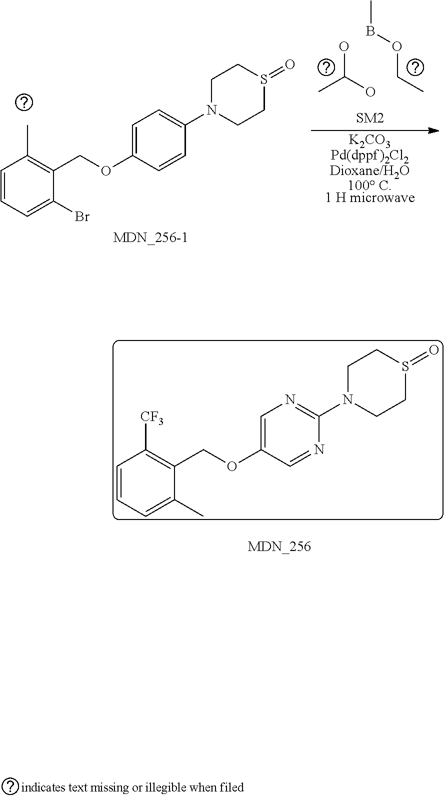

A mixture of MDN_256-1 (62 mg, 0.13 mmol), SM2 (3.5M in THF, 0.2 mL, 0.67 mmol), K₂CO₃ (56 mg, 0.40 mmol) and Pd (dppf)2Cl2 (20 mg, 0.03 mmol) in Dioxane/H2O (1.5 mL/0.3 mL) was stirred at 120° C. in microwave for 1 h. The mixture was quenched with water, extracted with EA, dried over Na2SO4, purified by silica gel column (DCM:MeOH=10:1) to give the title compound MDN_256 as a white solid (6 mg, 12% yield).

### Example 126

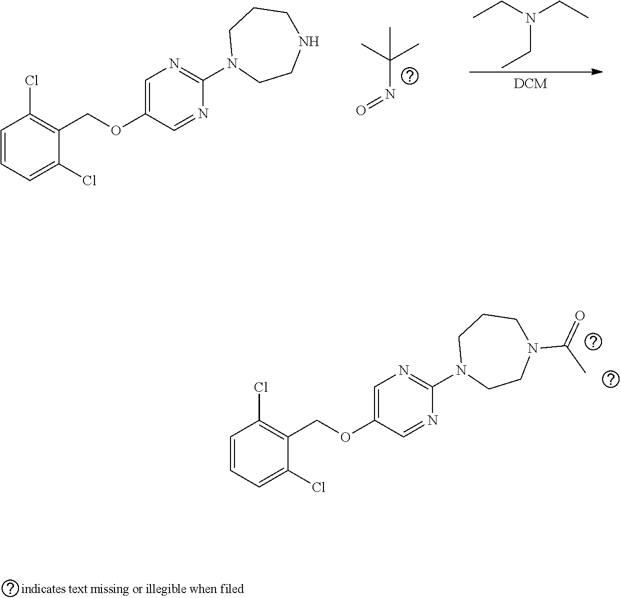

Trimethylsilyl isocyanate, 94% (0.03 mL, 0.85 g/mL, 0.26 mmol, 1.5 eq) was added to a stirred solution of 1-{5-[(2,6-dichlorophenyl) methoxy]pyrimidin-2-yl}-1,4-diazepane (60.4 mg, 0.17 mmol, 1 eq) and TRIETHYLAMINE (0.07 mL, 0.73 g/mL, 0.51 mmol, 3 eq) in DCM (4 mL). The reaction was stirred at rt overnight. The solvent was removed in vacuo then the resultant residue dissolved in DMSO, filtered and purified by preparative HPLC automated chromatography (ISCO ACCQ HP150, Prep HPLC Column: Gemini-NX C18 Dimensions: 21 mm×150 mm 5 μM; Sample: 1.50 ml from tube 1) eluting with 5 to 95% ACN/0.1% FA in Water/0.1% FA. Fractions 5 & 6 were combined then freeze dried. Gave 4-{5-[(2,6-dichlorophenyl) methoxy]pyrimidin-2-yl}-1,4-diazepane-1-carboxamide (34.78 mg, 0.09 mmol, 51.33%) as a white solid (Purity: >95%).

### Example 127

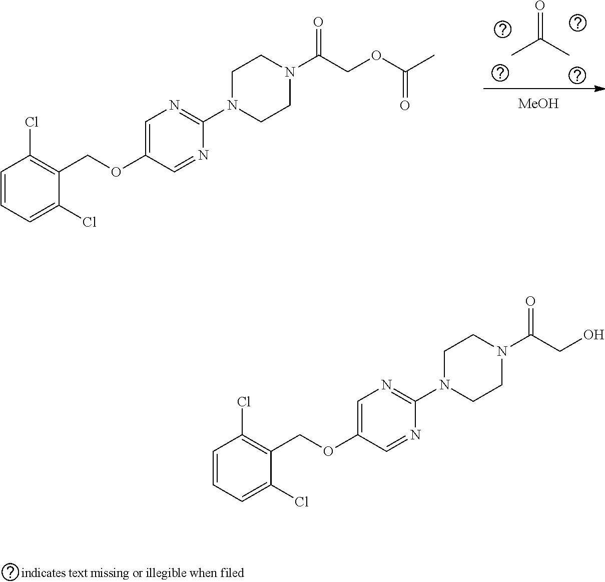

POTASSIUM CARBONATE (40.9 mg, 0.3 mmol, 10 eq) was added to a stirred solution of 2-(4-{5-[(2,6-dichlorophenyl) methoxy]pyrimidin-2-yl}piperazin-1-yl)-2-oxoethyl acetate (13 mg, 0.03 mmol, 1 eq) in Methanol (2 mL). The reaction was stirred at rt overnight. The reaction was diluted with DCM then washed with water. the aq was further extracted with DCM then the organic extracts combined, washed with brine, dried (MgSO4) and the solvent removed in vacuo. The resultant residue was dissolved in DMSO then filtered and purified by preparative HPLC automated chromatography (ISCO ACCQ HP150, Sample: 1.50 ml from tube 1; Prep HPLC Column: Gemini-NX C18 Dimensions: 21 mm×150 mm 5 μM) eluting with 5 to 95% ACN/0.1% FA in Water/0.1% FA. Dimensions: 21 mm×150 mm 5 μM) eluting with 5 to 95% ACN/0.1% FA in Water/0.1% FA. Fractions 3 & 4 were combined then freeze dried. Gave 1-(4-{5-[(2,6-dichlorophenyl) methoxy]pyrimidin-2-yl}piperazin-1-yl)-2-hydroxyethanone (4.06 mg, 0.01 mmol, 34.54%) as a white solid (Purity: >95%).

### Example 128

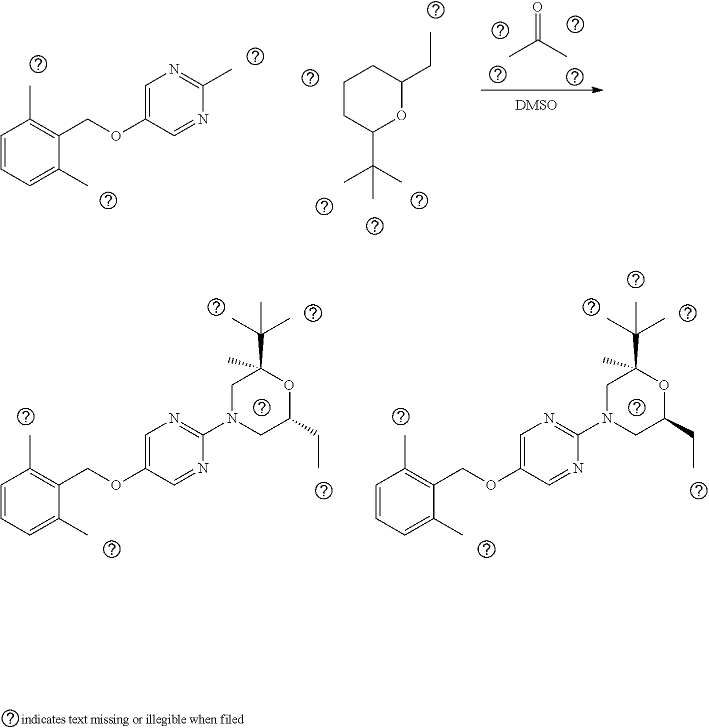

Nitrogen was bubbled through a DMSO (2 mL) solution of 2-chloro-5-[(2,6-dichlorophenyl) methoxy]pyrimidine (100 mg, 0.35 mmol, 1 eq), [6-(trifluoromethyl) morpholin-2-yl]methanol (63.94 mg, 0.35 mmol, 1 eq) and POTASSIUM CARBONATE (143.2 mg, 1.04 mmol, 3 eq). The tube was sealed then heated to 100° C., where it was maintained for ca 24 h. The reaction was cooled to rt then diluted with EtOAc and washed with water followed by brine. The organic extract was dried (MgSO4) then the solvent removed in vacuo. The resultant residue was dissolved in DMSO then filtered and purified by preparative HPLC automated chromatography (ISCO ACCQ HP150, Sample: 1.40 ml from tube 1; Prep HPLC Column: Big Gemini-NX C18 Dimensions: 30 mm×250 mm 5 μM) eluting with 5 to 95% ACN/0.1% FA in Water/0.1% FA Frac 44 was freeze dried (3527-RJP-196-001). The resultant glass was transferred to a submission vial using DCM/MeOH then the solvent removed under a stream of nitrogen. The compound was dried under vacuum at 40oC overnight. Gave [(2S,6S)-4-{5-[(2,6-dichlorophenyl) methoxy]pyrimidin-2-yl}-6-(trifluoromethyl) morpholin-2-yl]methanol+[(2R,6R)-4-{5-[(2,6-dichlorophenyl) methoxy]pyrimidin-2-yl}-6-(trifluoromethyl) morpholin-2-yl]methanol (8.34 mg, 0.02 mmol, 5.51%) as a colourless glass (Purity: >95%) (METHANOL, 5.5 eq). Due to the large amount of MeOH present the glass was dissolved in DCM then the solvent removed and the sample dried under vacuum at 40 overnight. Gave [(2S,6S)-4-{5-[(2,6-dichlorophenyl) methoxy]pyrimidin-2-yl}-6-(trifluoromethyl) morpholin-2-yl]methanol (4.25 mg, 0.01 mmol, 2.81%) as a colourless glass (Purity: 90-95%).

### Example 129

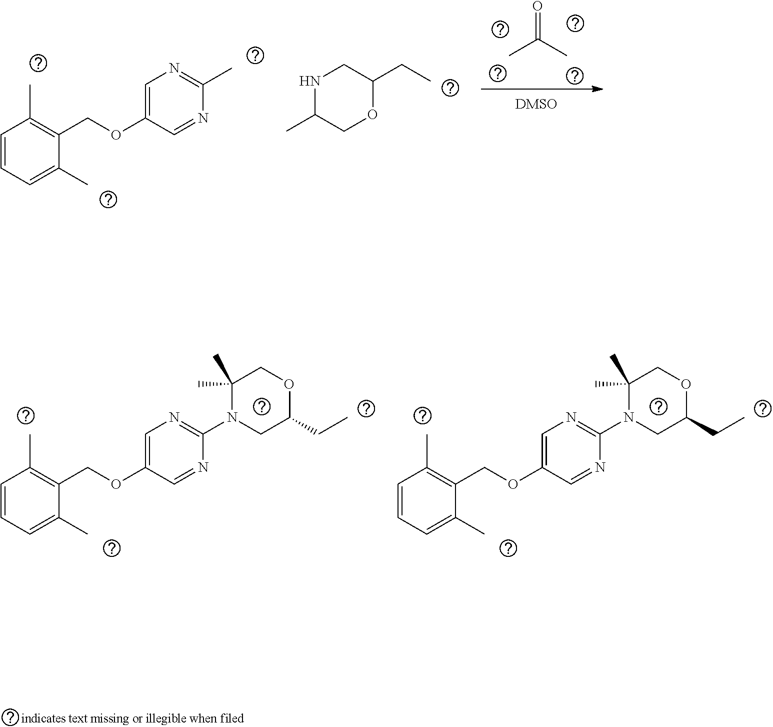

Nitrogen was bubbled through a DMSO (2 mL) solution of 2-chloro-5-[(2,6-dichlorophenyl) methoxy]pyrimidine, (100 mg, 0.35 mmol, 1 eq), (5-methylmorpholin-2-yl) methanol (45.3 mg, 0.35 mmol, 1 eq) and POTASSIUM CARBONATE (143.2 mg, 1.04 mmol, 3 eq). The tube was sealed then heated to 100° C., where it was maintained for ca 24 h. The reaction was cooled to rt then diluted with EtOAc and washed with water followed by brine. The organic extract was dried (MgSO4) then the solvent removed in vacuo. The resultant residue was dissolved in DMSO then filtered and purified by preparative HPLC automated chromatography (ISCO ACCQ HP150, Sample: 1.40 ml from tube 1; Prep HPLC Column: Big Gemini-NX C18 Dimensions: 30 mm×250 mm 5 μM) eluting with 5 to 95% ACN/0.1% FA in Water/0.1% FA Fraction 42-43 (trans) were combined then freeze dried. The resultant glass was transferred to a submission vial using DCM then the solvent removed under a stream of nitrogen. The compound was dried under vacuum at 40oC overnight. The compound was shown to be trans by NMR. Gave [(2S,5S)-4-{5-[(2,6-dichlorophenyl) methoxy]pyrimidin-2-yl}-5-methylmorpholin-2-yl]methanol+ [(2R,5R)-4-{5-[(2,6-dichlorophenyl) methoxy]pyrimidin-2-yl}-5-methylmorpholin-2-yl]methanol (2.6 mg, 0.01 mmol, 1.96%) as a colourless glass (Purity: >95%).

### Example 130

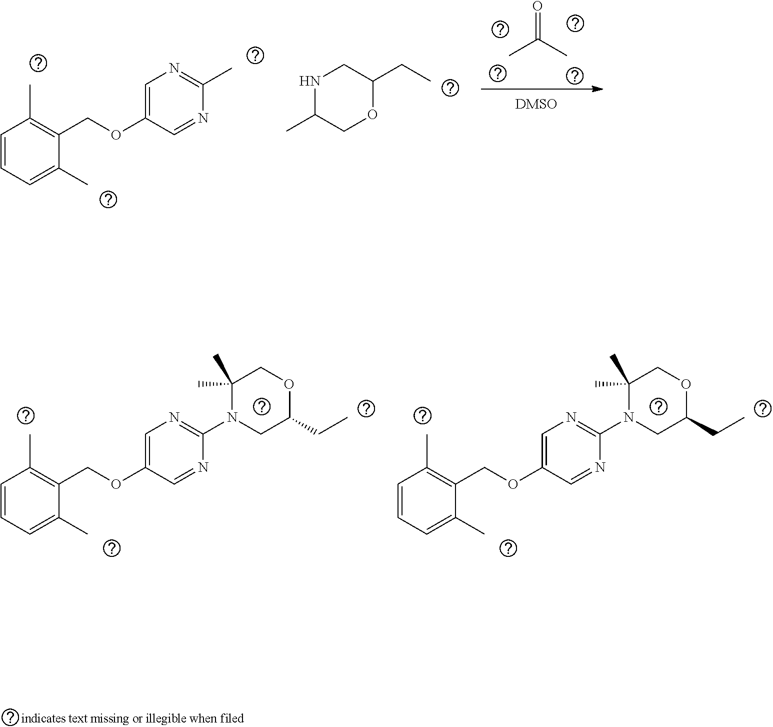

Nitrogen was bubbled through a DMSO (2 mL) solution of 2-chloro-5-[(2,6-dichlorophenyl) methoxy]pyrimidine (100 mg, 0.35 mmol, 1 eq), (5-methylmorpholin-2-yl) methanol (45.3 mg, 0.35 mmol, 1 eq) and POTASSIUM CARBONATE (143.2 mg, 1.04 mmol, 3 eq). The tube was sealed then heated to 100° C., where it was maintained for ca 24 h.

The reaction was cooled to rt then diluted with EtOAc and washed with water followed by brine. The organic extract was dried (MgSO4) then the solvent removed in vacuo. The resultant residue was dissolved in DMSO then filtered and purified by preparative HPLC automated chromatography (ISCO ACCQ HP150, Sample: 1.40 ml from tube 1; Prep HPLC Column: Big Gemini-NX C18 Dimensions: 30 mm×250 mm 5 μM) eluting with 5 to 95% ACN/0.1% FA in Water/0.1% FA Fraction 42-43 (trans) were combined then freeze dried. The resultant glass was transferred to a submission vial using DCM then the solvent removed under a stream of nitrogen. The compound was dried under vacuum at 40oC overnight. The compound was shown to be trans by NMR. Gave [(2S,5S)-4-{5-[(2,6-dichlorophenyl) methoxy]pyrimidin-2-yl}-5-methylmorpholin-2-yl]methanol+ [(2R,5R)-4-{5-[(2,6-dichlorophenyl) methoxy]pyrimidin-2-yl}-5-methylmorpholin-2-yl]methanol (2.6 mg, 0.01 mmol, 1.96%) as a colourless glass (Purity: >95%).

### Example 132

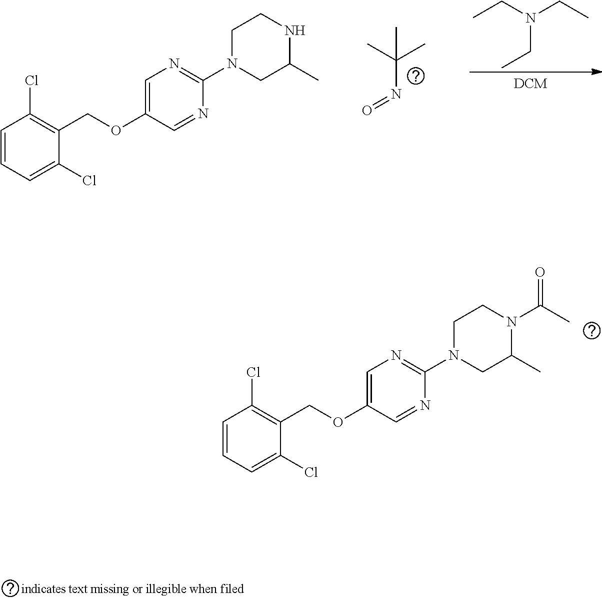

A solution of 5-[(2,6-dichlorophenyl) methoxy]-2-(3-methylpiperazin-1-yl) pyrimidine (45 mg, 0.13 mmol, 1 eq), Trimethylsilyl isocyanate, 94% (0.03 mL, 0.85 g/mL, 0.19 mmol, 1.5 eq) and TRIETHYLAMINE (0.05 mL, 0.73 g/mL, 0.38 mmol, 3 eq) in DCM (4 mL) was stirred at rt overnight. The solvent was removed in vacuo then the resultant residue dissolved in DMSO, filtered and purified by preparative HPLC automated chromatography (ISCO ACCQ HP150, Prep HPLC Column: Gemini-NX C18 Dimensions: 21 mm×150 mm 5 μM; Sample: 1.40 ml from tube 1) eluting with 5 to 95% ACN/0.1% FA in Water/0.1% FA. Fractions 8 & 9 were combined then freeze dried. gave 4-{5-[(2,6-dichlorophenyl) methoxy]pyrimidin-2-yl}-2-methylpiperazine-1-carboxamide (20.88 mg, 0.05 mmol, 41.36%) as a white solid (Purity: >95%).

### Example 133

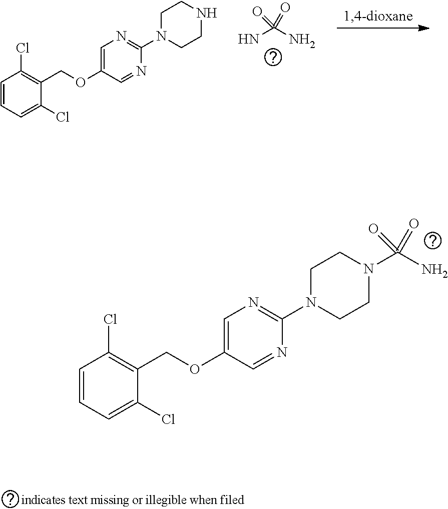

4-(aminosulfonyl)-1,1-dimethylethyl ester-1-piperazinecarboxylic acid To a solution of 1,1-dimethylethyl ester-1-piperazinecarboxylic acid (2.94 g) in dioxane (40 mL) was added sulfamide (4.0 g). The reaction mixture was then heated at reflux for 24 h. The reaction mixture was allowed to cool before being reduced in vacuo. The residue was separated between EtOAc (300 mL) and H₂O (300 mL) and the aqueous was further extracted (2×300 mL) with EtOAc. Combined organic layers were dried (MgSO4), filtered and evaporated. The residue was purified by column chromatography on silica gel using EtOAc/isohexane (1:1) as eluent to give the subtitle SULFAMIDE (27.99 mg, 0.29 mmol, 2.6 eq) was added to a stirred solution of 5-[(2,6-dichlorophenyl) methoxy]-2-(piperazin-1-yl)pyrimidine (38 mg, 0.11 mmol, 1 eq) in 1,4-Dioxane (2 mL). The reaction was heated to reflux, where it was maintained overnight. The reaction was cooled to rt then the solvent removed in vacuo. The resultant residue was dissolved in DMSO then filtered and purified by preparative HPLC automated chromatography (ISCO ACCQ HP150, Sample: 1.40 ml from tube 1; Prep HPLC Column: Gemini-NX C18 Dimensions: 21 mm×150 mm 5 μM) eluting with 5 to 95% ACN/0.1% FA in Water/0.1% FA. Fractions 8 & 9 were combined then the solvent removed in vacuo. Gave 4-{5-[(2,6-dichlorophenyl) methoxy]pyrimidin-2-yl}piperazine-1-sulfonamide (17.08 mg, 0.04 mmol, 36.45%) as a white solid (Purity: >95%).

### Example 134

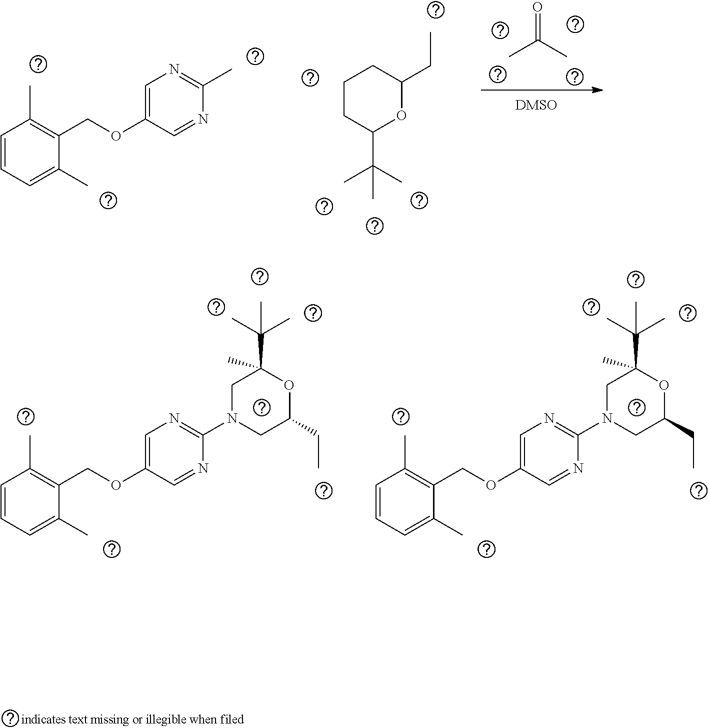

Nitrogen was bubbled through a DMSO (2 mL) solution of 2-chloro-5-[(2,6-dichlorophenyl) methoxy]pyrimidine (100 mg, 0.35 mmol, 1 eq), [6-(trifluoromethyl) morpholin-2-yl]methanol (63.94 mg, 0.35 mmol, 1 eq) and POTASSIUM CARBONATE (143.2 mg, 1.04 mmol, 3 eq). The tube was sealed then heated to 100° C., where it was maintained for ca 24 h. The reaction was cooled to rt then diluted with EtOAc and washed with water followed by brine. The organic extract was dried (MgSO4) then the solvent removed in vacuo. The resultant residue was dissolved in DMSO then filtered and purified by preparative HPLC automated chromatography (ISCO ACCQ HP150, Sample: 1.40 ml from tube 1; Prep HPLC Column: Big Gemini-NX C18 Dimensions: 30 mm×250 mm 5 μM) eluting with 5 to 95% ACN/0.1% FA in Water/0.1% FA Frac 44 was freeze dried (3527-RJP-196-001). The resultant glass was transferred to a submission vial using DCM/MeOH then the solvent removed under a stream of nitrogen. The compound was dried under vacuum at 40oC overnight. The compound was shown to be trans by NMR. Gave [(2S,6S)-4-{5-[(2,6-dichlorophenyl) methoxy]pyrimidin-2-yl}-6-(trifluoromethyl) morpholin-2-yl]methanol+ [(2R,6R)-4-{5-[(2,6-dichlorophenyl) methoxy]pyrimidin-2-yl}-6-(trifluoromethyl) morpholin-2-yl]methanol (8.34 mg, 0.02 mmol, 5.51%) as a colourless glass (3527-RJP-196-001, Purity: >95%) (METHANOL, 5.5 eq). Due to the large amount of MeOH present the glass was dissolved in DCM then the solvent removed and the sample dried under vacuum at 40oC overnight. Gave [(2S,6S)-4-{5-[(2,6-dichlorophenyl) methoxy]pyrimidin-2-yl}-6-(trifluoromethyl) morpholin-2-yl]methanol (4.25 mg, 0.01 mmol, 2.81%) as a colourless glass (Purity: 90-95%). Frac 50-52 were combined then freeze dried. The resultant solid was transferred to a submission vial. The compound was shown to be cis by NMR. Gave [(2R,6S)-4-{5-[(2,6-dichlorophenyl) methoxy]pyrimidin-2-yl}-6-(trifluoromethyl) morpholin-2-yl] methanol+ [(2S, 6R)-4-{5-[(2,6-dichlorophenyl) methoxy]pyrimidin-2-yl}-6-(trifluoromethyl) morpholin-2-yl]methanol (16.32 mg, 0.04 mmol, 10.78%) as a white solid (Purity: 90-95%).

### Example 135

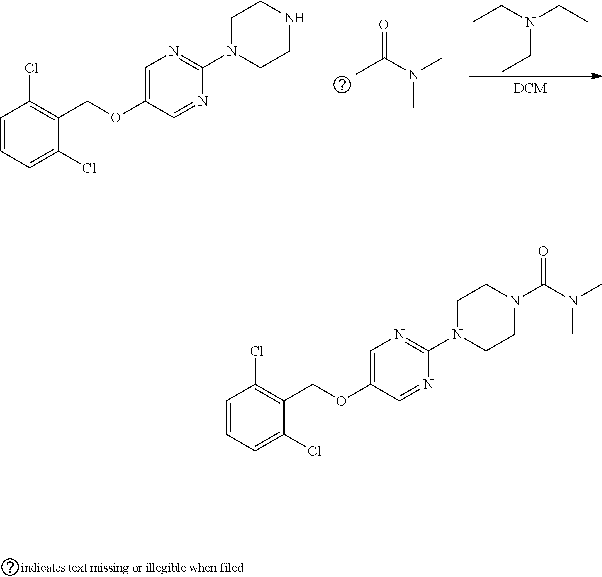

DIMETHYLCARBAMYL CHLORIDE (0.02 mL, 1.17 g/mL, 0.18 mmol, 2 eq) was added slowly to a stirred solution of 5-[(2,6-dichlorophenyl) methoxy]-2-(piperazin-1-yl) pyrimidine (30 mg, 0.09 mmol, 1 eq) and TRIETHYLAMINE (0.03 mL, 0.73 g/mL, 0.22 mmol, 2.5 eq) in DCM (2 mL) under a nitrogen atmosphere. The reaction was stirred at rt overnight. LCMS showed a mixture of product and starting material. Further TRIETHYLAMINE (0.03 mL, 0.73 g/mL, 0.22 mmol, 2.5 eq) and DIMETHYLCARBAMYL CHLORIDE (0.02 mL, 1.17 g/mL, 0.18 mmol, 2 eq) were added then stirring continued at rt over the weekend. The solvent was removed in vacuo then DMSO (0.5 ml) added. The solid was removed by filtration, washing with further DMSO. The filtrate was purified by preparative HPLC automated chromatography (ISCO ACCQ HP150, Sample: 1.80 ml from tube 1; Prep HPLC Column: GeminiNX C18 Dimensions: 21 mm×150 mm 5 μM) eluting with 5 to 95% ACN/0.1% FA in Water/0.1% FA. Fractions 17 & 18 were combined then freeze dried. The resultant glass was dissolved in DCM then transferred to a submission vial and the solvent removed. The compound was then dried overnight under vacuum at 40° C. Gave 4-{5-[(2,6-dichlorophenyl) methoxy]pyrimidin-2-yl}-N,N-dimethylpiperazine-1-carboxamide (11.04 mg, 0.03 mmol, 30.42%) as a white solid (Purity: >95%).

### Example 136

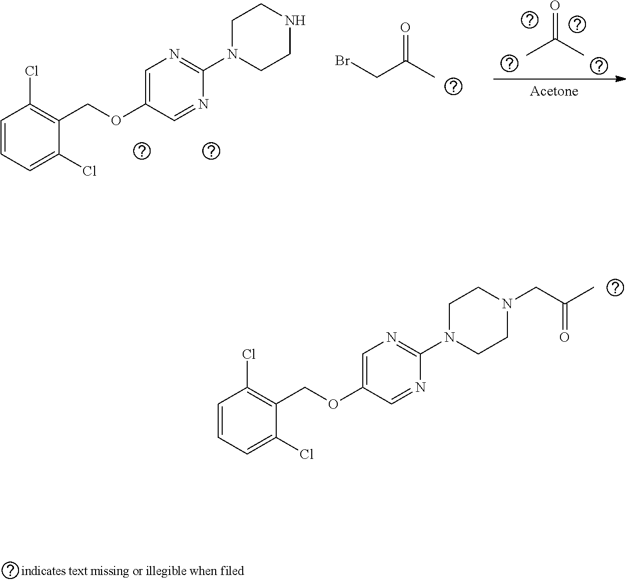

2-Bromoacetamide, 98% (20.08 mg, 0.15 mmol, 1.2 eq) was added to a stirred solution of 5-[(2,6-dichlorophenyl) methoxy]-2-(piperazin-1-yl)pyrimidine dihydrochloride (50 mg, 0.12 mmol, 1 eq) and POTASSIUM CARBONATE (83.83 mg, 0.61 mmol, 5 eq) in Acetone (3 mL). The reaction was heated to reflux, where it was maintained for ca 3 h. LCMS shows the reaction has gone to completion. The reaction was cooled to rt then the solid removed by filtration, washing with further acetone. The solvent was removed in vacuo then the resultant residue dissolved in DMSO and purified by preparative HPLC automated chromatography (ISCO ACCQ HP150, Prep HPLC Column: Gemini-NX C18 Dimensions: 21 mm×150 mm 5 μM; Sample: 1.40 ml from tube 1) eluting with 5 to 95% ACN/0.1% FA in Water/0.1% FA. Fractions 2-5 were combined then freeze dried. Gave 2-(4-{5-[(2,6-dichlorophenyl) methoxy]pyrimidin-2-yl}piperazin-1-yl) acetamide (24.42 mg, 0.06 mmol, 50.8%) as a white solid (Purity: >95%).

### Example 137

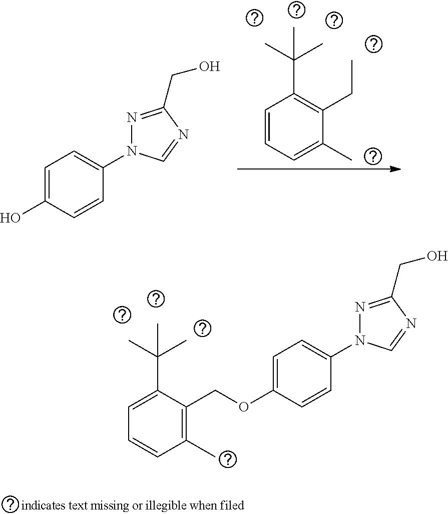

To a suspension of the starting compound above (40 mg, 0.21 mmol, 1 eq) in DMF (5 mL) was added 2-Chloro-6-(trifluoromethyl)benzyl bromide (41.29 μL, 1.66 g/cm³, 0.25 mmol, 1.2 eq) cesium carbonate (204.51 mg, 0.63 mmol, 3 eq) at rt under N₂. Stirred at rt overnight Work-up: The reaction mixture was diluted with EtOAc (20 mL) and water (20 mL). The organic layer was separated, washed with brine, dried over MgSO4 and conc in vacuum. Purification hplc ph 7 fractions Sep. 11, 2012 to give: [1-(4-{[2-chloro-6-(trifluoromethyl)phenyl] methoxy}phenyl)-1,2,4-triazol-3-yl]methanol (42 mg, 0.11 mmol, 52.31%) white solid (Purity: 90-95%)

### Example 138

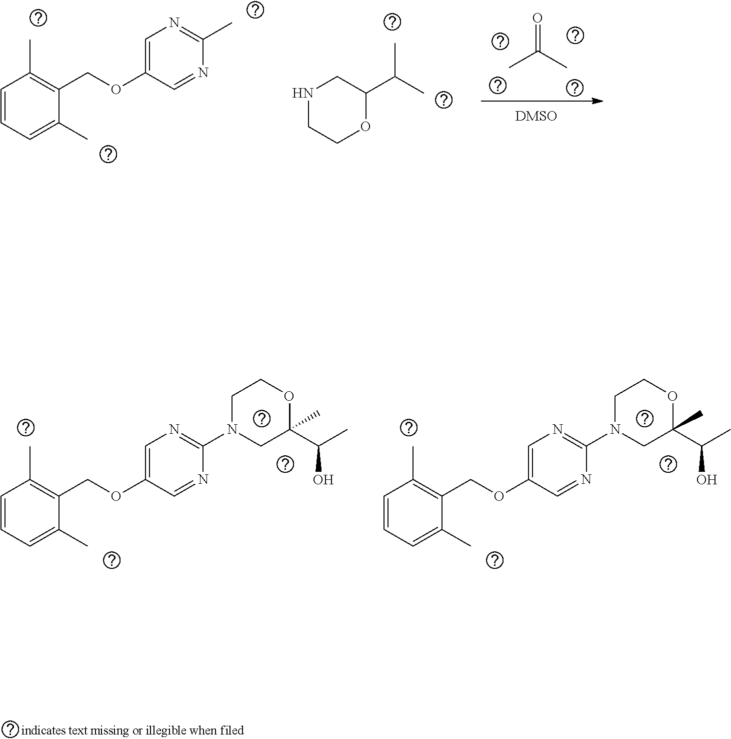

Nitrogen was bubbled through a DMSO (2 mL) solution of 2-chloro-5-[(2,6-dichlorophenyl) methoxy]pyrimidine (100 mg, 0.35 mmol, 1 eq), 1-(morpholin-2-yl) ethan-1-ol (45.3 mg, 0.35 mmol, 1 eq), and POTASSIUM CARBONATE (143.2 mg, 1.04 mmol, 3 eq). The tube was sealed then heated to 100° C., where it was maintained for ca 24 h. The reaction was cooled to rt then diluted with EtOAc and washed with water followed by brine. The organic extract was dried (MgSO4) then the solvent removed in vacuo. The resultant residue was dissolved in DMSO then filtered and purified by preparative HPLC automated chromatography (ISCO ACCQ HP150, Sample: 1.40 ml from tube 1; Prep HPLC Column: Big Gemini-NX C18 Dimensions: 30 mm×250 mm 5 μM) eluting with 5 to 95% ACN/0.1% FA in Water/0.1% FA Fractions 31-33 were combined then freeze dried. The resultant glass was transferred to a submission vial using DCM then the solvent removed under a stream of nitrogen. The compound was dried under vacuum at 40oC overnight. Shown to be a mix of SS and RR by NMR and modelling. Gave (1R)-1-[(2R)-4-{5-[(2,6-dichlorophenyl) methoxy]pyrimidin-2-yl}morpholin-2-yl]ethanol (30.66 mg, 0.08 mmol, 23.1%) as a white glass (Purity: >95%). Fractions 35-36 were combined then freeze dried. The resultant glass was transferred to a submission vial using DCM/MeOH then the solvent removed under a stream of nitrogen. The compound was dried under vacuum at 40° C. overnight. Shown to be a mix of SR and RS by NMR and modelling. Gave (1R)-1-[(2S)-4-{5-[(2,6-dichlorophenyl) methoxy]pyrimidin-2-yl}morpholin-2-yl]ethanol (17.43 mg, 0.05 mmol, 13.13%) as a colourless glass (Purity: 90-95%) (METHANOL, 2.3 eq). Due to the significant amount of MeOH then compound was dissolved in minimal DCM then the solvent removed. The sample was dried overnight at 40° C. under vacuum. Gave (1R)-1-[(2S)-4-{5-[(2,6-dichlorophenyl) methoxy]pyrimidin-2-yl}morpholin-2-yl]ethanol (16.52 mg, 0.04 mmol, 12.45%) as a colourless glass (Purity: 90-95%).

### Example 139

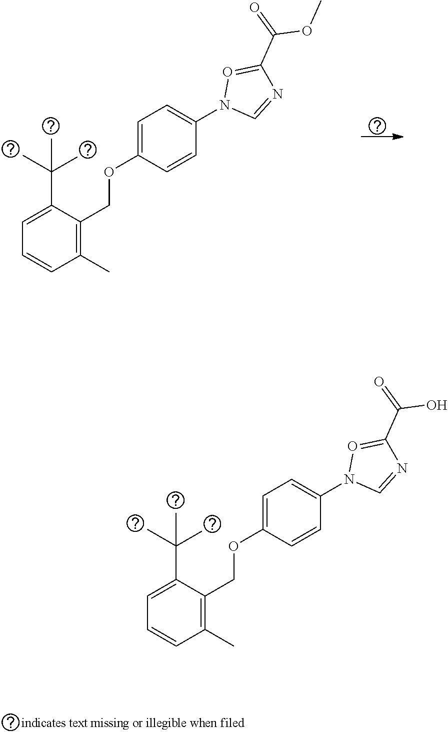

To a solution of the above ester (88 mg, 0.22 mmol, 1 eq) in THF (2 mL) Methanol (1 mL), Water (0.5 mL), was added LithiumHydroxide Monohydrate (47.18 mg, 1.12 mmol, 5 eq) at rt. Stirred overnight at rt. The reaction mixture was conc in vacuum. Dissolved into DMSO (2 mL), filtered and purified by preparative HPLC automated chromatography (ISCO ACCQ HP150, Prep HPLC Column: Big Gemini-NX C18 Dimensions: 30 mm×250 mm 5 μM; Sample: 2.40 ml from tube 1) eluting with 5 to 95% ACN/0.1% FA in Water/0.1% FA fractions 6-12 to give: 1-(4-{[2-methyl-6-(trifluoromethyl)phenyl] methoxy}phenyl)-1,2,4-triazole-3-carboxylic acid (48 mg, 0.13 mmol, 56.57%) white solid (Purity: >95%)

### Example 140

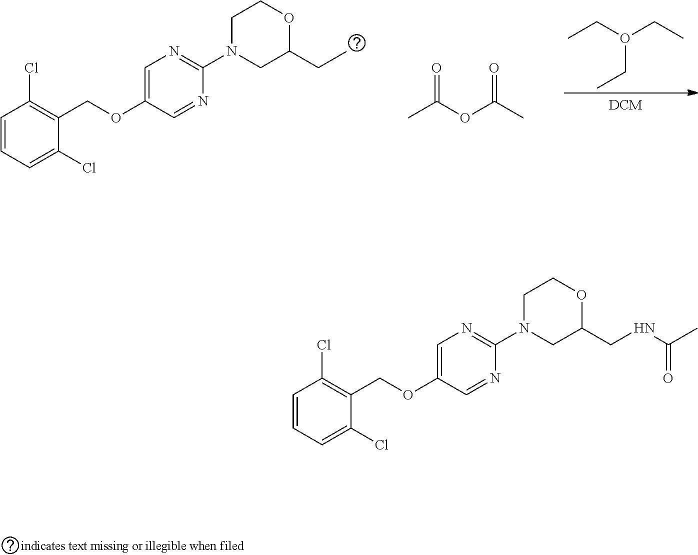

Acetic anhydride, 99+% (6.25 μL, 1.08 g/mL, 0.07 mmol, 1.5 eq) was added to a stirred solution of (4-{5-[(2,6-dichlorophenyl) methoxy]pyrimidin-2-yl}morpholin-2-yl) methanamine (16.3 mg, 0.04 mmol, 1 eq) and TRIETHYLAMINE (0.02 mL, 0.73 g/mL, 0.13 mmol, 3 eq) in DCM (2 mL). The reaction was stirred at rt overnight. The solvent was removed in vacuo then the resultant residue dissolved in DMSO, filtered and purified by preparative HPLC automated chromatography (ISCO ACCQ HP150, Prep HPLC Column: Gemini-NX C18 Dimensions: 21 mm×150 mm 5 μM; Sample: 1.20 ml from tube 1) eluting with 5 to 95% ACN/0.1% FA in Water/0.1% FA. Fraction 14 was freeze dried giving N-[(4-{5-[(2,6-dichlorophenyl) methoxy]pyrimidin-2-yl}morpholin-2-yl)methyl]acetamide (6.19 mg, 0.02 mmol, 34.09%) as a white solid (Purity: >95%).

### Example 141

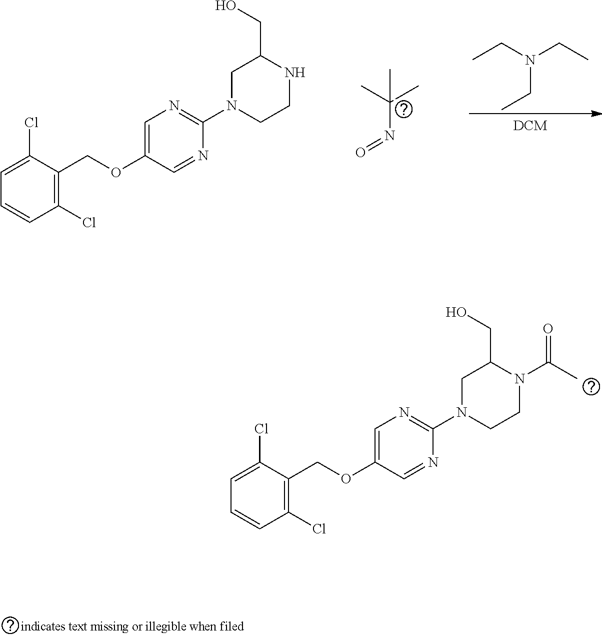

Trimethylsilyl isocyanate, 94% (0.03 mL, 0.85 g/mL, 0.25 mmol, 1.5 eq) was added to a stirred solution of (4-{5-[(2,6-dichlorophenyl) methoxy]pyrimidin-2-yl}piperazin-2-yl) methanol (61 mg, 0.17 mmol, 1 eq) and TRIETHYLAMINE (0.07 mL, 0.73 g/mL, 0.5 mmol, 3 eq) in DCM (3 mL). The reaction was stirred at rt overnight. The solvent was removed in vacuo then the resultant residue dissolved in DMSO, filtered and purified by preparative HPLC automated chromatography (ISCO ACCQ HP150, Sample: 1.30 ml from tube 1; Prep HPLC Column: Gemini-NX C18 Dimensions: 21 mm×150 mm 5 M) eluting with 5 to 95% ACN/0.1% FA in Water/0.1% FA. Fractions 7 & 8 were combined then freeze dried. Gave 4-{5-[(2,6-dichlorophenyl) methoxy]pyrimidin-2-yl}-2-(hydroxymethyl) piperazine-1-carboxamide (27.98 mg, 0.07 mmol, 41.08%) as a white solid (Purity: >95%).

### Example 142

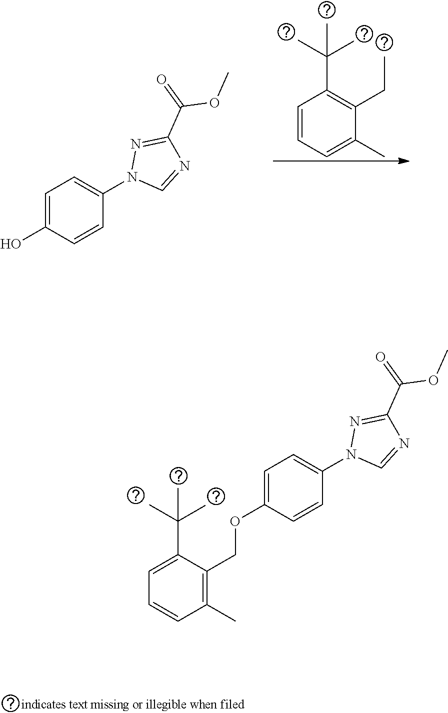

To a solution of the above starting material ester (100 mg, 0.46 mmol, 1 eq) in DMF (5 mL) was added 2-METHYL-6-TRIFLUOROMETHYLBENZYL BROMIDE, (72.16 μL, 1.6 g/cm³, 0.46 mmol, 1 eq) followed by cesium carbonate (297.29 mg, 0.91 mmol, 2 eq) at rt under n2. Stirred for 3 days at rt. The reaction mixture was diluted with EtOAc (50 mL) and water (50 mL). The organic layer was separated, washed with brine, dried over MgSO4 and conc in vacuum. purified by automated flash chromatography (Combiflash Rf, Silica 12 g RediSep column) eluting with 0 to 60% ethyl acetate in heptane fractions 7-11 to give: methyl 1-(4-{[2-methyl-6-(trifluoromethyl)phenyl] methoxy}phenyl)-1,2,4-triazole-3-carboxylate (107 mg, 0.27 mmol, 59.93%) white solid (Purity: 85-90%) LC-MS and NMR showed some impurity 19 mg were dissolved into DMSO (1.5 mL) and purified by preparative HPLC automated chromatography (ISCO ACCQ HP125, Prep HPLC Column: Gemini-NX C18 Dimensions: 21 mm×150 mm 5 μM; Sample: 1.90 ml from tube 1) eluting with 20 to 95% ACN/0.1% FA in Water/0. 1% FA fractions 1-3 to give: methyl 1-(4-{[2-methyl-6-(trifluoromethyl)phenyl] methoxy}phenyl)-1,2,4-triazole-3-carboxylate (12 mg, 0.03 mmol, 6.72%) white solid (Purity: >95%)

### Example 165

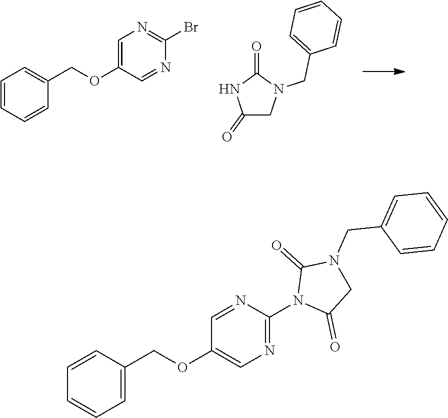 [SMILES: Brc1ncc(OCc2ccccc2)cn1 ; O=C1CN(Cc2ccccc2)C(=O)N1 ; O=C1CN(Cc2ccccc2)C(=O)N1c1ncc(OCc2ccccc2)cn1]

5-(benzyloxy)-2-bromopyrimidine (500 mg, 1.89 mmol, 1 eq)N-Benzylhydantoin (538.08 mg, 2.83 mmol, 1.5 eq) and COPPER (I) OXIDE (269.88 mg, 1.89 mmol, 1 eq) were diluted with DMF (3 mL) under nitrogen and stirred at 150° C. for 3 Hours The reaction mixture was filtered through celite, eluted with ethyl acetate, washed with a saturated aqueous solution of ammonium chloride (30 mL), extracted with ethyl acetate (30 mL), washed with 5% aqueous lithium chloride (2×30 mL), brine (30 mL), passed through hydrophobic filter paper, adsorbed onto Isolute HM-N, concentrated under reduced pressure and purified by automated flash chromatography (Combiflash Rf, Silica 24 g RediSep column) eluting with 12 to 100% ethyl acetate in heptane Fractions containing pure product by TLC (3:1 EtOAc: n-heptane) (5-8) were concentrated under reduced pressure to give 1-benzyl-3-[5-(benzyloxy) pyrimidin-2-yl]imidazolidine-2,4-dione (336 mg (69%), 0.62 mmol, 32.83%) as a cream gum (Purity: 50-85%) Fraction 3 had crystallised on standing over 48 hours. The crystals were filtered, washed with n-heptane and dried at 40° C. for 2 h to give 1-benzyl-3-[5-(benzyloxy) pyrimidin-2-yl]urea (16 mg, 0.05 mmol, 2.54%) as white crystals (Purity: >95%).

### Example 166

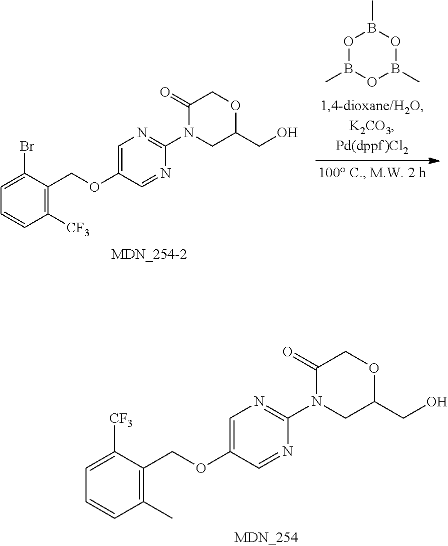

To a solution of MDN_254-2 (52 mg, 0.110 mmol) in 1,4-dioxane (4 mL) and H₂O (0.1 mL) were added 2,4,6-trimethyl-1,3,5,2,4,6-trioxatriborinane (0.5 mL), K2CO3 (47 mg, 0.340 mmol) and Pd (dppf) C₁₂ (17 mg, 0.02 mmol). The reaction was stirred at 100oC for 2 h in a microwave reactor.

The reaction mixture was added H₂O (30 mL) and extracted with EtOAc (30 mL×3). The organic layer was dried over Na2SO4, filtered and concentrated in vacuo. The crude product purified by prep-HPLC (FA) and lyophilized to afford the title compound as a white solid (6.77 mg, 10.9% yield).

### Example 167

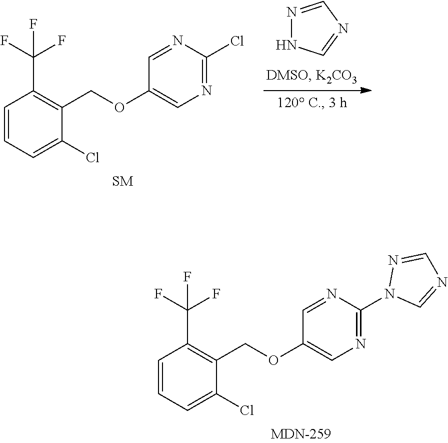 [SMILES: FC(F)(F)c1cccc(Cl)c1COc1cnc(Cl)nc1 ; c1nc[nH]n1 ; FC(F)(F)c1cccc(Cl)c1COc1cnc(-n2cncn2)nc1]

2-chloro-5-((2-chloro-6-(trifluoromethyl)benzyl)oxy) pyrimidine (50 mg, 0.155 mmol) and 1H-1,2,4-triazole (10 mg, 0.155 mmol) and K₂CO₃ (42 mg, 0.310 mmol) in DMSO (1 mL) was stirred at 120° C. under Ar for 3 h. The reaction mixture was diluted with H₂O (30 mL) and extracted with EtOAc (30 mL×3). The organic phase was washed with brine (80 mL×2), dried over Na2SO4, filtered and concentrated. The crude product was purified by chromatography on silica gel (EA/PE=0%˜50%) to give the title compound as a white solid (50 mg, 75.0% yield).

### Example 168

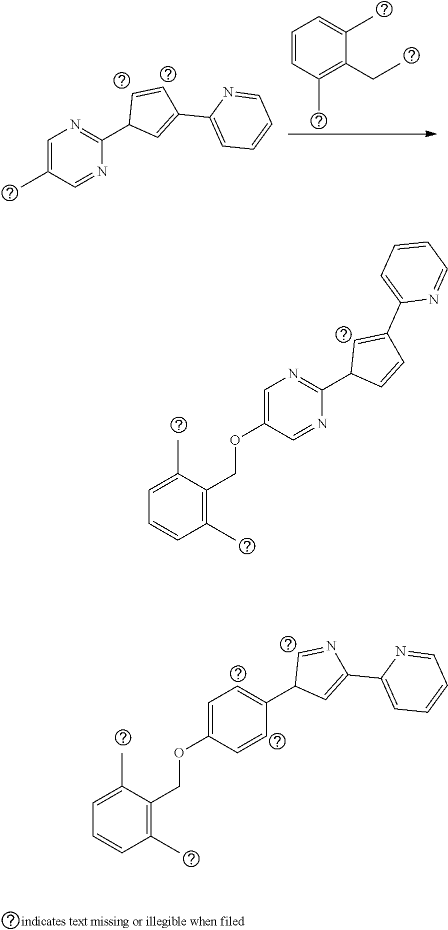

To a solution of the above starting material (36 mg, 0.15 mmol, 1 eq) in DMF (2 mL) was added 2,6-Dichlorobenzyl bromide (35.96 mg, 0 g/mL, 0.15 mmol, 1 eq) followed by potassium carbonate (41.42 mg, 0.3 mmol, 2 eq) The reaction mixture was heated at RT overnight. The reaction mixture was filtered and purified by preparative HPLC automated chromatography (ISCO ACCQ HP125, Prep HPLC Column: Big Gemini-NX C18 Dimensions: 30 mm×250 mm 5 μM; Sample: 2.20 ml from tube 1) eluting with 5 to 95% ACN/0.1% FA in Water/0.1% FA. fractions 62-64 gave 5-[(2,6-dichlorophenyl) methoxy]-2-[4-(pyridin-2-yl)-1,2,3-triazol-1-yl]pyrimidine (10 mg, 0.03 mmol, 16.71%) white solid (Purity: 90-95%)

### Example 170

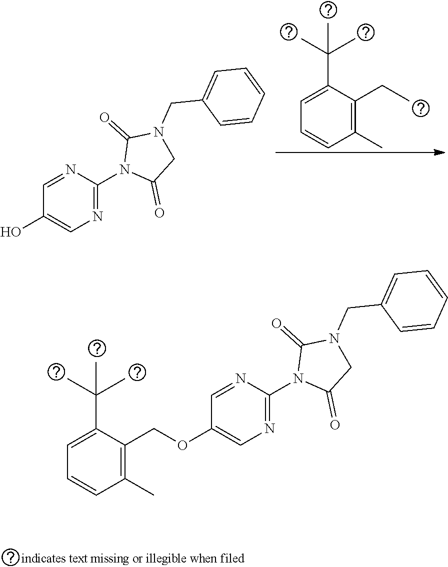

To 1-benzyl-3-(5-hydroxypyrimidin-2-yl) imidazolidine-2,4-dione (26.4 mg, 0.09 mmol, 1 eq) and potassium carbonate (64.17 mg, 0.46 mmol, 5 eq) was added a solution of 2-(Bromomethyl)-1-methyl-3-(trifluoromethyl)benzene (23.5 mg, 0.09 mmol, 1 eq) (liquid at r.t.) in DMF (1 mL) and the reaction stirred at 21° C. for 30 Mins. LCMS analysis showed complete consumption of starting material. The reaction was quenched with water (15 mL), extracted with ethyl acetate (2×15 mL), washed with brine (15 mL), passed through hydrophobic filter paper and concentrated under reduced pressure to give a colourless oil. The crude mixture was diluted with DMSO and purified by preparative HPLC automated chromatography (ISCO ACCQ HP125, Prep HPLC Column: Gemini-NX C18 Dimensions: 21 mm×150 mm 5 μM; Sample: 1.20 ml from tube 1) eluting with 5 to 95% ACN/0.1% FA in Water/0.1% FA Fractions containing pure desired product by LCMS (10-11) were concentrated and dried under vacuum at 40° C. overnight to give 1-benzyl-3-(5-{[2-methyl-6-(trifluoromethyl)phenyl] methoxy}pyrimidin-2-yl) imidazolidine-2,4-dione (11.3 mg, 0.02 mmol, 26.66%) as a white solid (Purity: >95%).

### Example 171

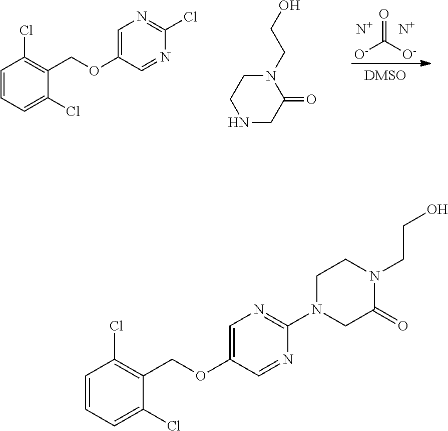

Nitrogen was bubbled through a DMSO (2 mL) solution of 2-chloro-5-[(2,6-dichlorophenyl) methoxy]pyrimidine (100 mg, 0.35 mmol, 1 eq), 1-(2-hydroxyethyl) piperazin-2-one (59.75 mg, 0.41 mmol, 1.2 eq) and POTASSIUM CARBONATE (143.2 mg, 1.04 mmol, 3 eq) in a pressure tube. The tube was sealed then heated to 100° C., where it was maintained overnight. LCMS shows a mixture of product and starting material. The reaction was cooled to rt then diluted with EtOAc and washed with sat. aq. NaHCO3 solution followed by brine. The organic extract was dried (MgSO4) then the solvent removed in vacuo. The resultant residue was dissolved in DMSO, filtered then purified by preparative HPLC automated chromatography (ISCO ACCQ HP150, Sample: 1.40 ml from tube 1; Prep HPLC Column: Gemini-NX C18 Dimensions: 21 mm×150 mm 5 M) eluting with 5 to 95% ACN/0.1% FA in Water/0.1% FA Fractions 14 & 15 were combined then freeze dried. The resultant residue was dissolved in DCM then transferred to a submission vial. The solvent was removed under a stream of nitrogen then the sample dried over the weekend under vacuum at 40° C. Gave 4-{5-[(2,6-dichlorophenyl) methoxy]pyrimidin-2-yl}-1-(2-hydroxyethyl) piperazin-2-one (26.02 mg, 0.07 mmol, 18.96%) as a white glass (Purity: >95%).

### Example 172

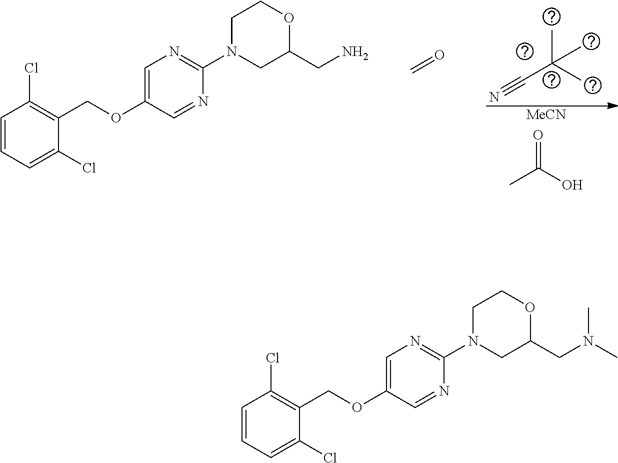

Sodium cyanoborohydride (25.53 mg, 0.41 mmol, 3 eq) was added to a stirred (cloudy) solution of (4-{5-[(2,6-dichlorophenyl) methoxy]pyrimidin-2-yl}morpholin-2-yl) methanamine (50 mg, 0.14 mmol, 1 eq) and formaldehyde (37% in water) (0.1 mL (37%), 1.35 mmol, 10 eq) in Acetonitrile (3 mL). Acetic acid, 99.5%, pure (0.05 mL, 1.05 g/mL, 0.87 mmol, 6.46 eq) was added (reaction goes clear) then the reaction stirred at rt for ca 2 h. The reaction was quenched with sat. aq. NaHCO3 solution then extracted with EtOAc (×2). The organic extract was washed with brine then dried (MgSO4) and the solvent removed in vacuo. The resultant residue was dissolved in DMSO, filtered and then purified by preparative HPLC automated chromatography (ISCO ACCQ HP150, Prep HPLC Column: Gemini-NX C18 Dimensions: 21 mm×150 mm 5 μM; Sample: 1.30 ml from tube 1) eluting with 5 to 95% ACN/0.1% FA in Water/0.1% FA automated chromatography (ISCO ACCQ HP150, Prep HPLC Column: Gemini-NX C18 Dimensions: 21 mm×150 mm 5 μM; Sample: 1.30 ml from tube 1) eluting with 5 to 95% ACN/0.1% FA in Water/0.1% FA. Fractions 4-7 were combined then freeze dried. The resultant residue was dissolved in DCM then transferred to a submission vial. The solvent was removed under a stream of nitrogen followed by the V10 then the sample dried over the weekend under vacuum at 40° C. Gave [(4-{5-[(2,6-dichlorophenyl) methoxy]pyrimidin-2-yl}morpholin-2-yl)methyl]dimethylamine (24.51 mg, 0.06 mmol, 45.56%) as a colourless gum (Purity: >95%).

### Example 173

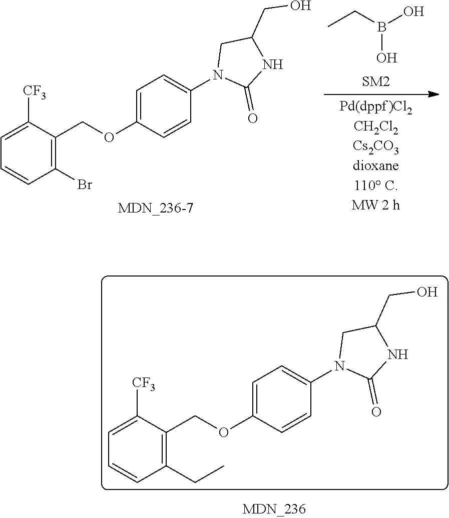

A mixture of MDN_236-7 (70 mg, 0.157 mmol), SM2 (35 mg, 0.471 mmol), Pd (dppf)2Cl2 CH2C12 (63 mg, 0.078 mmol) and Cs₂CO₃ (154 mg, 0.471 mmol) in Dioxane (2 mL) was stirred in microwave at 110° C. for 2 h. The mixture was filtered, concentrated, purified by silica gel column (DCM:MeOH=30:1) to give the title compound MDN_236 as a colorless oil (14 mg, 23% yield).

### Example 174

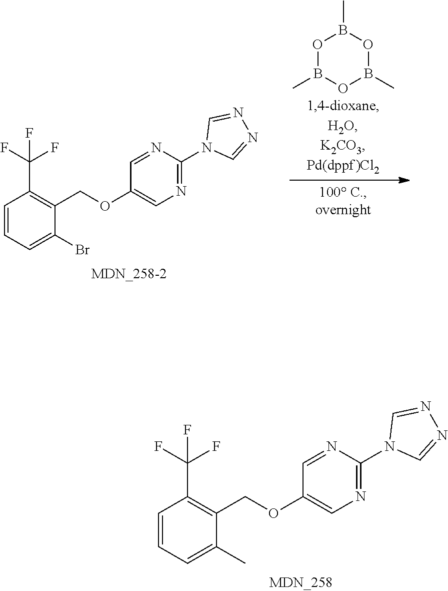

To a mixture of MDN_258-2 (76 mg, 0.190 mmol) in 1,4-dioxane/H₂O (1 mL/0.1 mL) were added 2,4,6-trimethyl-1,3,5,2,4,6-trioxatriborinane (0.5 mL), Pd (dppf) C₁₂ (27 mg, 0.038 mmol) and K₂CO₃ (77 mg, 0.570 mmol). The reaction was stirred at 100oC under Ar overnight. The reaction mixture was diluted with H₂O (50 mL) and extracted with EtOAc (50 mL×3). The organic phase dried over Na2SO4, concentrated and the crude product was purified by silica gel column to give the title compound as a yellow solid (20 mg, 31% yield).

### Example 175

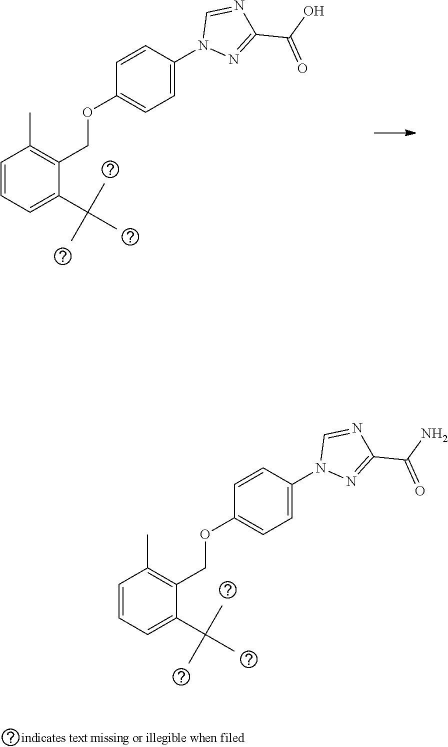 [SMILES: Cc1cccc(C(C)(C)C)c1COc1ccc(-n2cnc(C(=O)O)n2)cc1 ; Cc1cccc(C(C)(C)C)c1COc1ccc(-n2cnc(C(N)=O)n2)cc1]

To a solution of (39 mg, 0.1 mmol, 1 eq) in DMF (3 mL) was added Ammonium Chloride (27.64 mg, 0.52 mmol, 5 eq) Hunigs Base (0.14 mL, 0.83 mmol, 8 eq) HATU O-(7-Azabenzotriazol-1-yl)-N,N,N′,N′-tetramethyluronium hexafluorophosphate (78.6 mg, 0.21 mmol, 2 eq) at rt under n2. Stirred overnight at rt. The reaction mixture was conc in vacuum. Dissolved into DMSO (2.2 mL) and purified on HPLC at ph4 fractions 28-33 to give: 1-(4-{[2-methyl-6-(trifluoromethyl)phenyl] methoxy}phenyl)-1,2,4-triazole-3-carboxamide (20 mg, 0.05 mmol, 51.42%) white solid (Purity: >95%).

### Example 176

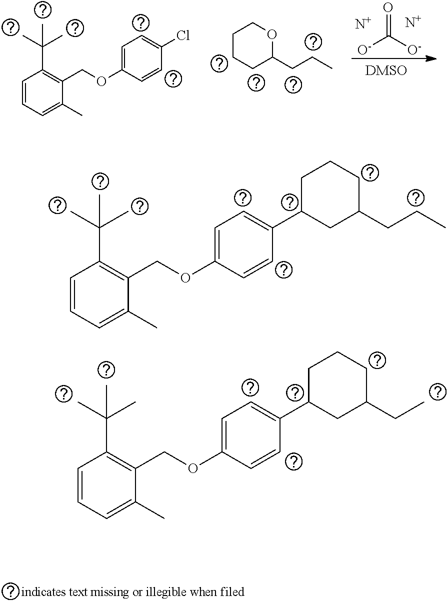

Nitrogen was bubbled through a DMSO (2 mL) solution of 2-chloro-5-{[2-methyl-6-(trifluoromethyl)phenyl] methoxy}pyrimidine (100 mg, 0.33 mmol, 1 eq), Methyl-morpholin-2-ylmethyl-amine dihydrochloride (100.66 mg, 0.5 mmol, 1.5 eq) and POTASSIUM CARBONATE (228.3 mg, 1.65 mmol, 5 eq) in a pressure tube. The tube was sealed then heated to 100° C., where it was maintained overnightThe reaction was cooled to rt then diluted with EtOAc and washed with sat. aq. NaHCO3 solution followed by brine. The organic extract was dried (MgSO4) then the solvent removed in vacuo. The resultant residue was dissolved in DMSO, filtered then purified by preparative HPLC automated chromatography (ISCO ACCQ HP150, Prep HPLC Column: Gemini-NX C18 Dimensions: 21 mm×150 mm 5 μM; Sample: 1.30 ml from tube 1) eluting with 5 to 95% ACN/0.1% FA in Water/0.1% FA. Fraction 4 was freeze dried (significant product also present in fraction 5 but impurity also present). Gave [4-(5-{[2-methyl-6-(trifluoromethyl)phenyl] methoxy}pyrimidin-2-yl) morpholin-2-yl]methanamine (32.06 mg, 0.08 mmol, 25.38%) as a white solid (Purity: >95%) (FORMATE, 0.83 eq).

### Example 179

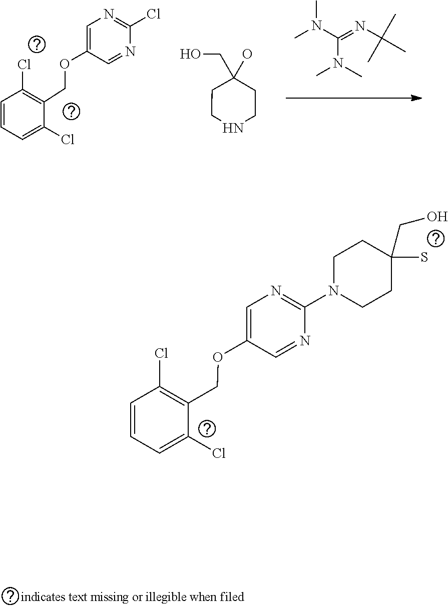 [SMILES: Clc1ncc(OCc2c(Cl)cccc2Cl)cn1 ; OCC1(O)CCNCC1 ; CN(C)C(=NC(C)(C)C)N(C)C ; OCC1(S)CCN(c2ncc(OCc3c(Cl)cccc3Cl)cn2)CC1]

To a stirred solution of 2-chloro-5-[(2,6-dichlorophenyl) methoxy]pyrimidine (100 mg, 0.35 mmol, 1 eq) in DMSO (3 mL) was added (4-fluoropiperidin-4-yl) methanol hydrochloride (70.3 mg (78.5%), 0.41 mmol, 1.2 eq) and 2-TERT-BUTYL-1,1,3,3-TETRAMETHYLGUANIDINE (171.97 μL, 0.86 g/mL, 0.86 mmol, 2.5 eq). Reaction was heated to 100° C. for 18 h. LCMS showed excellent conversion to product. Reaction was cooled to rt and poured into brine (100 mL). The mixture was extracted weith EtOAc (3×30 mL). Combined organics were washed (brine 2×20 mL), dried (phase sep paper) and concentrated to dryness affording a brown film. The film was purified by preparative HPLC automated flash chromatography (ISCO ACCQ HP150, Prep HPLC Column: Big Gemini-NX C18 Dimensions: 30 mm×250 mm 5 μM; Sample: 2.50 ml from tube 1 RediSep column) eluting with 5 to 95% ACN/0.1% FA in Water/0.1% FA Desired fractions were combined and concentrated to dryness affording (1-{5-[(2,6-dichlorophenyl) methoxy]pyrimidin-2-yl}-4-fluoropiperidin-4-yl) methanol (68 mg, 0.18 mmol, 50.98%) as a colourless gum (Purity: >95%).

### Example 180

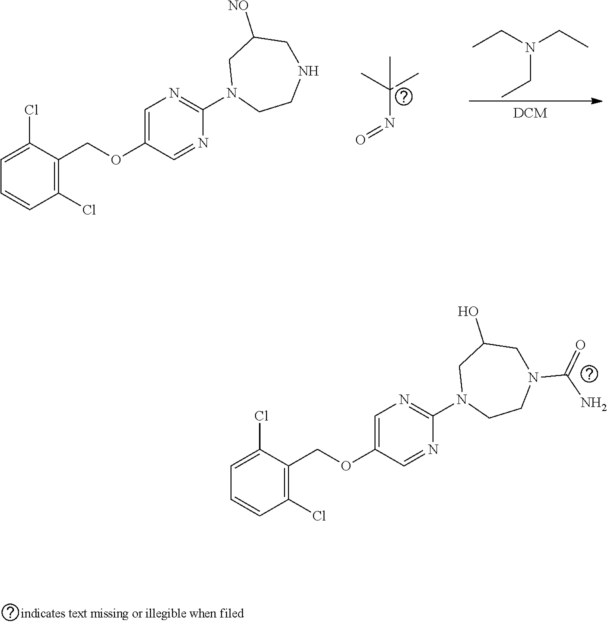

Trimethylsilyl isocyanate, 94% (0.03 mL, 0.85 g/mL, 0.19 mmol, 1.5 eq) was added to a stirred solution of 1-{5-[(2,6-dichlorophenyl) methoxy]pyrimidin-2-yl}-1,4-diazepan-6-ol (46.7 mg, 0.13 mmol, 1 eq) and TRIETHYLAMINE (0.05 mL, 0.73 g/mL, 0.38 mmol, 3 eq) in DCM (3 mL). The reaction was stirred at rt overnight. The solvent was removed in vacuo then the resultant residue dissolved in DMSO, filtered and purified by preparative HPLC automated chromatography (ISCO ACCQ HP150, Prep HPLC Column: Gemini-NX C18 Dimensions: 21 mm×150 mm 5 μM; Sample: 1.50 ml from tube 1) eluting with 5 to 95% ACN/0.1% FA in Water/0.1% FA. Fractions 5 & 6 were combined then freeze dried. Gave 4-{5-[(2,6-dichlorophenyl) methoxy]pyrimidin-2-yl}-6-hydroxy-1,4-diazepane-1-carboxamide (19.37 mg, 0.05 mmol, 37.15%) as a white solid (Purity: >95%).

### Example 181

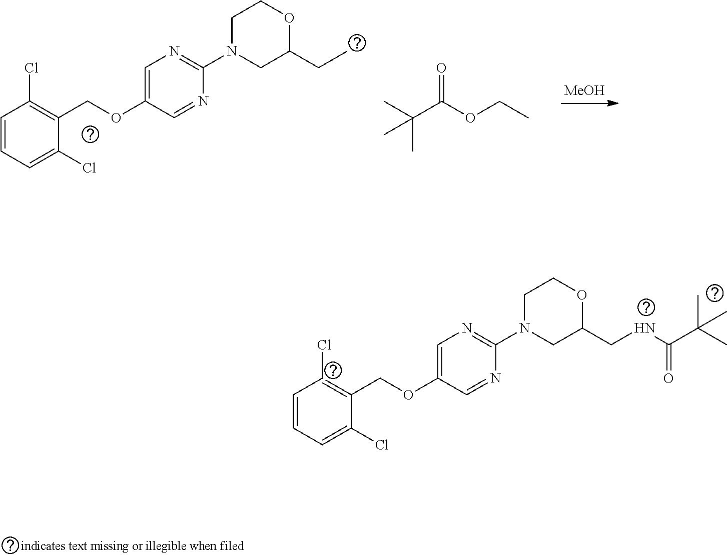

ETHYL TRIFLUOROACETATE (0.02 mL, 1.19 g/mL, 0.16 mmol, 1.2 eq) was added to a stirred solution of (4-{5-[(2,6-dichlorophenyl) methoxy]pyrimidin-2-yl}morpholin-2-yl) methanamine (48 mg, 0.13 mmol, 1 eq) in Methanol (2 mL). The reaction was stirred at rt for ca 1.5 h during which time a precipitate formed. The solvent was removed in vacuo then the resultant residue dissolved in DMSO, filtered and purified by preparative HPLC automated chromatography (ISCO ACCQ HP150, Prep HPLC Column: Gemini-NX C18 Dimensions: 21 mm×150 mm 5 μM; Sample: 1.30 ml from tube 1) eluting with 5 to 95% ACN/0.1% FA in Water/0.1% FA. Fractions 16-18 were combined then freeze dried. Gave N-[(4-{5-[(2,6-dichlorophenyl) methoxy]pyrimidin-2-yl}morpholin-2-yl)methyl]-2,2,2-trifluoroacetamide (24.9 mg, 0.05 mmol, 41.17%) as a white solid (Purity: >95%).

### Example 182

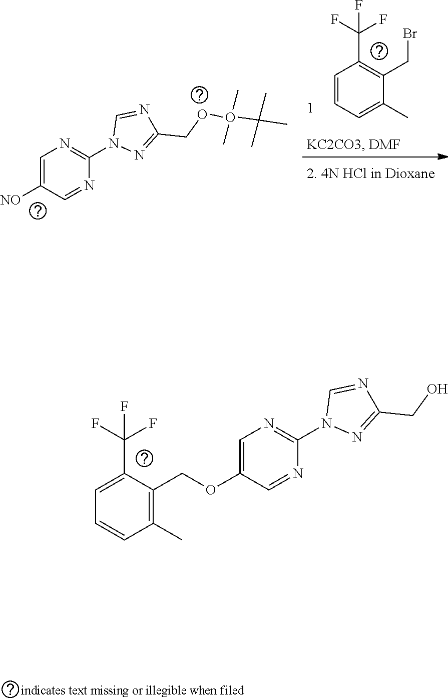

To a solution of the above silyl ether (110 mg, 0.36 mmol, 1 eq) in DMF (2 mL) was added 2-METHYL-6-TRIFLUOROMETHYLBENZYL BROMIDE (56.59 μL, 1.6 g/cm³, 0.36 mmol, 1 eq) and potassium carbonate (98.9 mg, 0.72 mmol, 2 eq) at rt under N₂. Stirred at rt overnight. The reaction mixture was diluted with EtOAc (5 mL), filtered and conc in vacuum. HCl (4M in Dioxane) (2 mL) was added and stirred at RT for 2 hours. The reaction mixture was conc in vacuum, dissolved into DMSO (3.4 mL) and purified by preparative HPLC automated chromatography (ISCO ACCQ HP125, Sample: 1.70 ml from tube 1; Prep HPLC Column: Big Gemini-NX C18 Dimensions: 30 mm×250 mm 5 μM) eluting with 20 to 95% ACN/0.1% FA in Water/0.1% FA fractions 4-6 and 14-16 to give: [1-(5-{[2-methyl-6-(trifluoromethyl)phenyl] methoxy}pyrimidin-2-yl)-1,2,4-triazol-3-yl]methanol (93 mg, 0.25 mmol, 71.15%) white solid (Purity: >98%).

### Example 183

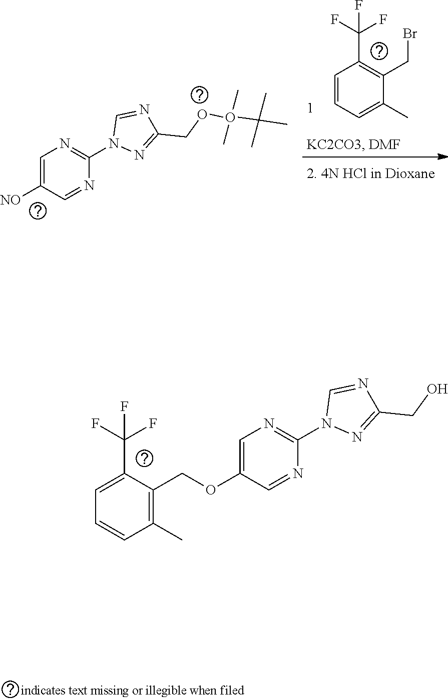

To a solution of the above silyl ether (110 mg, 0.36 mmol, 1 eq) in DMF (2 mL) was added 2-Chloro-6-(trifluoromethyl)benzyl bromide (61.16 μL, 1.6 g/cm³, 0.36 mmol, 1 eq) and potassium carbonate (98.9 mg, 0.72 mmol, 2 eq) at rt under N₂. Stirred at rt overnight Work-up: The reaction mixture was diluted with EtOAc (5 mL), filtered and conc in vacuum. HCl (4M in Dioxane) (2 mL) was added and stirred at RT for 2 hours. The reaction mixture was conc in vacuum on genevac, dissolved into DMSO (3.4 mL) and purified by preparative HPLC automated chromatography (ISCO ACCQ HP125, Prep HPLC Column: Big Gemini-NX C18 Dimensions: 30 mm×250 mm 5 μM; Sample: 1.70 ml from tube 1) eluting with 20 to 95% ACN/0.1% FA in Water/0.1% FA. Fractions 6-8 and 18-20 [1-(5-{[2-chloro-6-(trifluoromethyl)phenyl] methoxy}pyrimidin-2-yl)-1,2,4-triazol-3-yl]methanol (109 mg, 0.28 mmol, 78.98%) white solid (Purity: >95%).

### Example 185

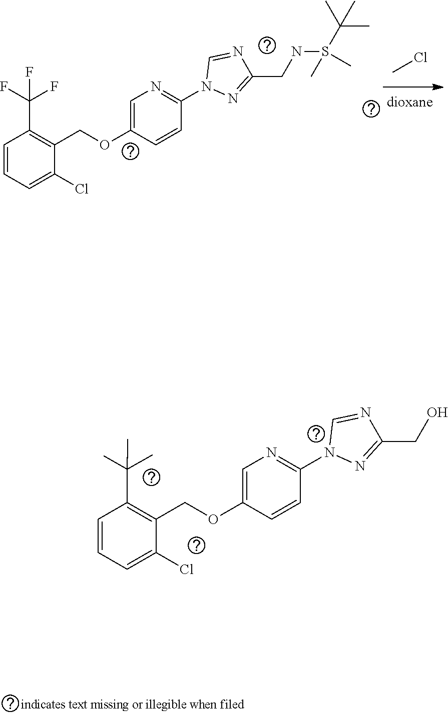

2-(3-{[(tert-butyldimethylsilyl)oxy] methyl}-1,2,4-triazol-1-yl)-5-{[2-chloro-6-(trifluoromethyl)phenyl] methoxy}pyridine (156 mg, 0.31 mmol, 1 eq) was taken up in 1,4-Dioxane (1 mL) and hydrochloric acid (2.97 mL, 4M in, 11.88 mmol, 38 eq) (in dioxane) was added. Rxn was stirred at rt for 2 h, conc in vacuo, then taken up in 2 mL DMSO and filtered. purified by preparative HPLC automated chromatography (ISCO ACCQ HP150, Prep HPLC Column: Big Gemini-NX C18 Dimensions: 30 mm×250 mm 5 μM; Sample: 2.30 ml from tube 1) eluting with 5 to 95% ACN/0.1% FA in Water/0.1% FA 250 mm 5 μM; Sample: 2.30 ml from tube 1) eluting with 5 to 95% ACN/0.1% FA in Water/0.1% FA F23 to F29 were combined and frozen on a CO₂/acetone bath and freeze dried. 62.29 mg isolated as a white solid to give [1-(5-{[2-chloro-6-(trifluoromethyl)phenyl] methoxy}pyridin-2-yl)-1,2,4-triazol-3-yl]methanol (62.29 mg, 0.16 mmol, 51.79%).

### Example 186

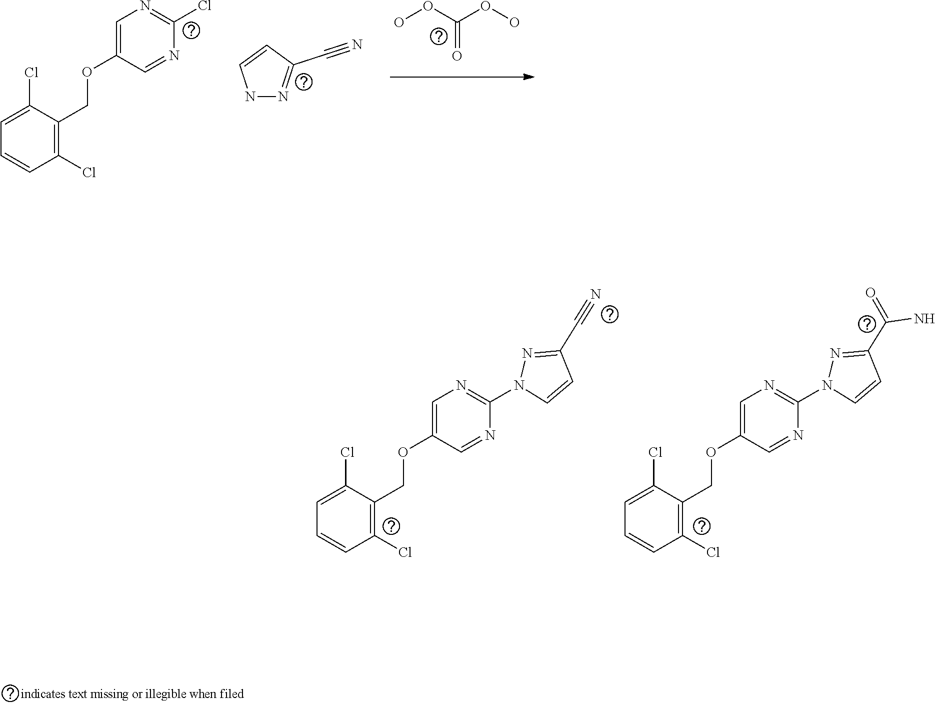 [SMILES: Clc1ncc(OCc2c(Cl)cccc2Cl)cn1 ; N#Cc1cc[nH]n1 ; O=C(OO)OO ; N#Cc1ccn(-c2ncc(OCc3c(Cl)cccc3Cl)cn2)n1 ; NC(=O)c1ccn(-c2ncc(OCc3c(Cl)cccc3Cl)cn2)n1]

To a stirred solution of 2-chloro-5-{[2-methyl-6-(trifluoromethyl)phenyl] methoxy}pyrimidine (150 mg, 0.52 mmol, 1 eq) in DMSO (3 mL) was added 1H-PYRAZOLE-3-CARBONITRILE (57.87 mg, 0.62 mmol, 1.2 eq) and POTASSIUM CARBONATE (107.4 mg, 0.78 mmol, 1.5 eq). Reaction was sealed and heated to 110° C. for 18 h. Reaction was cooled to rt and filtered through a plug of celite. The resulting mixture was directly purified by preparative HPLC automated flash chromatography (ISCO ACCQ HP125, Prep HPLC Column: Big Gemini-NX C18 Dimensions: 30 mm×250 mm 5 μM; Sample: 2.50 ml from tube 1 RediSep column) eluting with 5 to 95% ACN/0.1% FA in Water/0.1% FA. Desired fractions (40-48) were combined and concentrated to dryness affording 1-{5-[(2,6-dichlorophenyl) methoxy]pyrimidin-2-yl}pyrazole-3-carbonitrile (70.9 mg, 0.2 mmol, 39.53%) as a white powder (Purity: >95%).

### Example 187

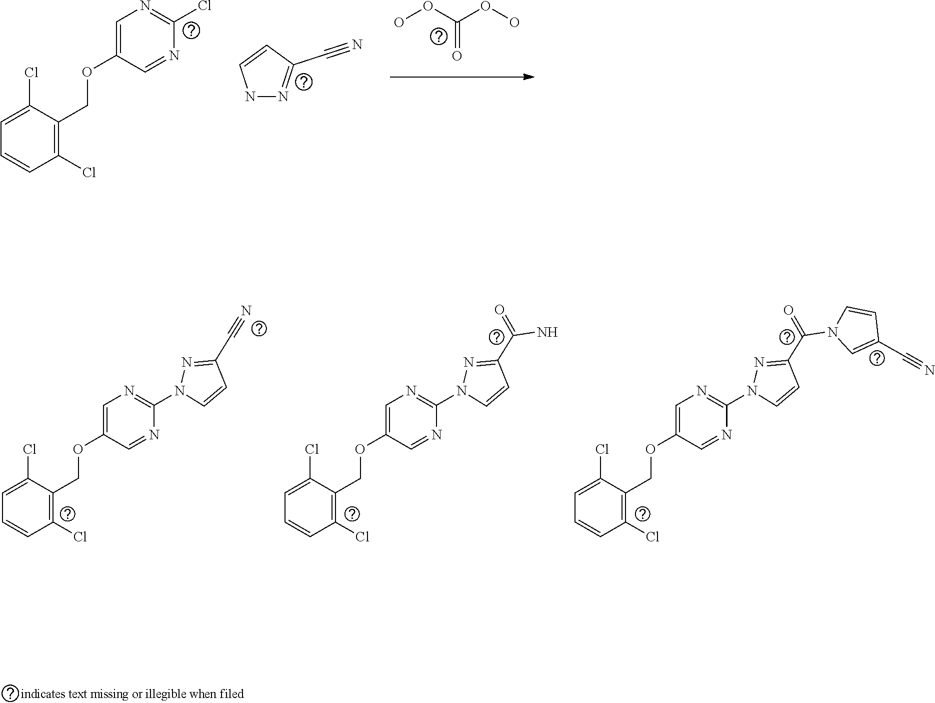 [SMILES: Clc1ncc(OCc2c(Cl)cccc2Cl)cn1 ; N#Cc1cc[nH]n1 ; O=C(OO)OO ; N#Cc1ccn(-c2ncc(OCc3c(Cl)cccc3Cl)cn2)n1 ; NC(=O)c1ccn(-c2ncc(OCc3c(Cl)cccc3Cl)cn2)n1 ; N#Cc1ccn(C(=O)c2ccn(-c3ncc(OCc4c(Cl)cccc4Cl)cn3)n2)c1]

To a stirred solution of 2-chloro-5-[(2,6-dichlorophenyl) methoxy]pyrimidine (150 mg, 0.52 mmol, 1 eq) in DMSO (3 mL) with POTASSIUM CARBONATE (107.4 mg, 0.78 mmol, 1.5 eq) was added 3-Cyano-1,2,4-triazole (58.48 mg, 0.62 mmol, 1.2 eq). Reaction was sealed and heated to 110° C. for 18 h. Reaction was cooled to rt and filtered through a plug of celite. The mixture was directly purified by preparative HPLC automated flash chromatography (ISCO ACCQ HP125, Sample: 2.50 ml from tube 1; Prep HPLC Column: Big Gemini-NX C18 Dimensions: 30 mm×250 mm 5 μM RediSep column) eluting with 5 to 95% ACN/0.1% FA in Water/0.1% FA Desired fractions (35-38) were combined and concentrated to dryness affording 1-{5-[(2,6-dichlorophenyl) methoxy]pyrimidin-2-yl}-1,2,4-triazole 3-carbonitrile (5.11 mg, 0.01 mmol, 2.84%) as a white powder (Purity: >95%).

### Example 188

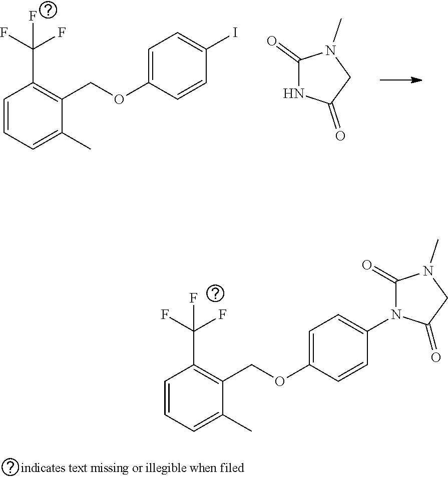

2-(4-iodophenoxymethyl)-1-methyl-3-(trifluoromethyl)benzene (100 mg, 0.26 mmol, 1 eq) 1-Methylhydantoin, 97% (43.64 mg, 0.38 mmol, 1.5 eq) and COPPER (I) OXIDE (36.49 mg, 0.26 mmol, 1 eq) were diluted with DMF (0.65 mL) under nitrogen and stirred at 150° C. for 16 Hours. The reaction mixture was filtered through celite, eluting with ethyl acetate (40 mL), washed with a saturated aqueous solution of ammonium chloride (30 mL), 5% aqueous lithium chloride (2×20 mL), passed through hydrophobic filter paper, concentrated under reduced pressure to give a white solid (88.5 mg) and purified by preparative HPLC automated chromatography (ISCO ACCQ HP125, Prep HPLC Column: Big Gemini-NX C18 Dimensions: 30 mm×250 mm 5 μM; Sample: 2.50 ml from tube 1) eluting with 20 to 95% ACN/0.1% FA in Water/0.1% FA uM; Sample: 2.50 ml from tube 1) eluting with 20 to 95% ACN/0.1% FA in Water/0.1% FA. Fractions containing pure desired product by LCMS (7-13) were lyophilised to give 1-methyl-3-(4-{[2-methyl-6-(trifluoromethyl)phenyl] methoxy}phenyl) imidazolidine-2,4-dione (44.5 mg, 0.12 mmol, 46.12%) as a white solid (Purity: >95%) (FORMATE, 0.056 eq).

### Example 189

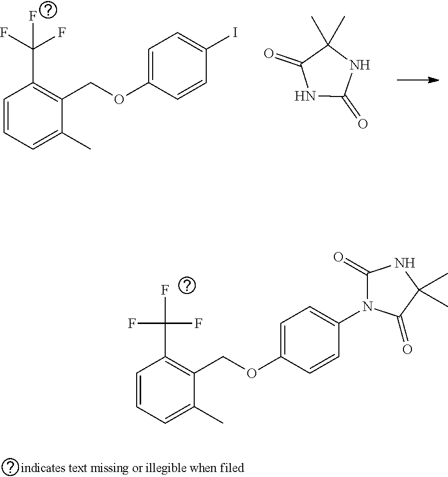

2-(4-iodophenoxymethyl)-1-methyl-3-(trifluoromethyl)benzene (100 mg, 0.26 mmol, 1 eq) 5,5-Dimethylhydantoin (49.01 mg, 0.38 mmol, 1.5 eq) and COPPER (I) OXIDE (36.49 mg, 0.26 mmol, 1 eq) were diluted with DMF (0.65 mL) under nitrogen and stirred at 150° C. for 16. The reaction mixture was filtered through celite, eluting with ethyl acetate (40 mL), washed with a saturated aqueous solution of ammonium chloride (30 mL), 5% aqueous lithium chloride (2×20 mL), passed through hydrophobic filter paper, concentrated under reduced pressure to give a yellow solid (97.6 mg), diluted with DMSO and purified by preparative HPLC automated chromatography (ISCO ACCQ HP125, Prep HPLC Column: Big Gemini-NX C18 Dimensions: 30 mm×250 mm 5 μM; Sample: 2.50 ml from tube 2) eluting with 20 to 95% ACN/0.1% FA in Water/0.1% FA uM; Sample: 2.50 ml from tube 2) eluting with 20 to 95% ACN/0.1% FA in Water/0.1% FA Fractions containing pure desired product by LCMS (1-7) were lyophilised to give 5,5-dimethyl-3-(4-{[2-methyl-6-(trifluoromethyl)phenyl] methoxy}phenyl) imidazolidine-2,4-dione (53.3 mg, 0.14 mmol, 53.27%) as a white solid (Purity: >95%).

### Example 190

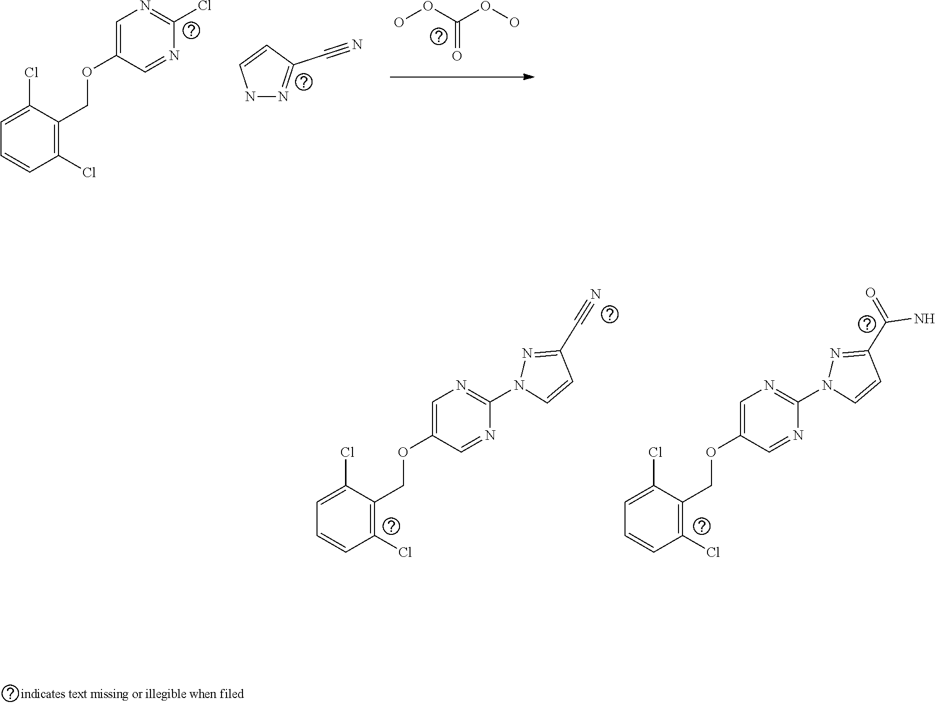 [SMILES: Clc1ncc(OCc2c(Cl)cccc2Cl)cn1 ; N#Cc1cc[nH]n1 ; O=C(OO)OO ; N#Cc1ccn(-c2ncc(OCc3c(Cl)cccc3Cl)cn2)n1 ; NC(=O)c1ccn(-c2ncc(OCc3c(Cl)cccc3Cl)cn2)n1]

To a stirred solution of 2-chloro-5-{[2-methyl-6-(trifluoromethyl)phenyl] methoxy}pyrimidine (150 mg, 0.52 mmol, 1 eq) in DMSO (3 mL) was added 1H-PYRAZOLE-3-CARBONITRILE (57.87 mg, 0.62 mmol, 1.2 eq) and POTASSIUM CARBONATE (107.4 mg, 0.78 mmol, 1.5 eq. Reaction was sealed and heated to 110° C. for 18 h. Reaction was cooled to rt and filtered through a plug of celite. The resulting mixture was directly purified by preparative HPLC automated flash chromatography (ISCO ACCQ HP125, Prep HPLC Column: Big Gemini-NX C18 Dimensions: 30 mm×250 mm 5 μM; Sample: 2.50 ml from tube 1 RediSep column) eluting with 5 to 95% ACN/0.1% FA in Water/0.1% FA Desired fractions (40-48) were combined and concentrated to dryness affording 1-{5-[(2,6-dichlorophenyl) methoxy]pyrimidin-2-yl}pyrazole-3-carbonitrile (70.9 mg, 0.2 mmol, 39.53%) as a white powder (Purity: >95%) Further fractions pertaining to the amide (24-27) were combined and concentrated to dryness affording 8 mg of a white powder. Analysis showed amide and related impurities. The powder was re purified by automated flash chromatography (Combiflash 300+, Silica 4 g RediSep column) eluting with 0 to 100% Ethyl Acetate in Heptane Acetate in Heptane Desired fractions were combined and concentrated to dryness affording 1-{5-[(2,6-dichlorophenyl) methoxy]pyrimidin-2-yl}pyrazole-3-carboxamide (2.33 mg, 0.01 mmol, 1.23%) as a white powder (Purity: 90-95%).

### Example 191

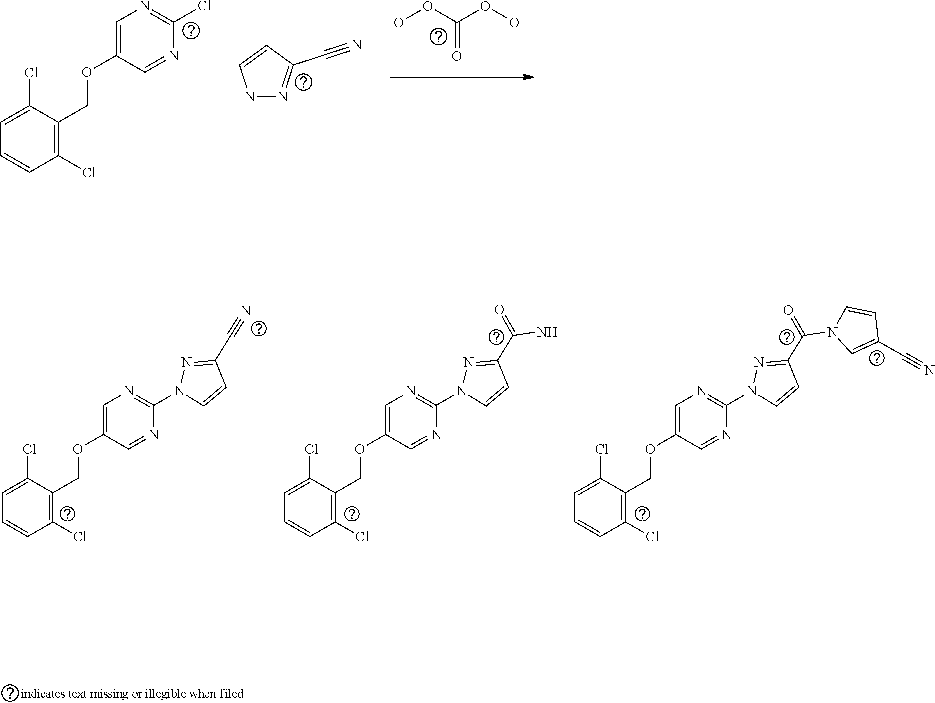 [SMILES: Clc1ncc(OCc2c(Cl)cccc2Cl)cn1 ; N#Cc1cc[nH]n1 ; O=C(OO)OO ; N#Cc1ccn(-c2ncc(OCc3c(Cl)cccc3Cl)cn2)n1 ; NC(=O)c1ccn(-c2ncc(OCc3c(Cl)cccc3Cl)cn2)n1 ; N#Cc1ccn(C(=O)c2ccn(-c3ncc(OCc4c(Cl)cccc4Cl)cn3)n2)c1]

To a stirred solution of 2-chloro-5-[(2,6-dichlorophenyl) methoxy]pyrimidine (150 mg, 0.52 mmol, 1 eq) in DMSO (3 mL) with POTASSIUM CARBONATE (107.4 mg, 0.78 mmol, 1.5 eq) was added 3-Cyano-1,2,4-triazole (58.48 mg, 0.62 mmol, 1.2 eq). Reaction was sealed and heated to 110° C. for 18 h. Reaction was cooled to rt and filtered through a plug of celite. The mixture was directly purified by preparative HPLC automated flash chromatography (ISCO ACCQ HP125, Sample: 2.50 ml from tube 1; Prep HPLC Column: Big Gemini-NX C18 Dimensions: 30 mm×250 mm 5 μM RediSep column) eluting with 5 to 95% ACN/0.1% FA in Water/0.1% FA Desired fractions (35-38) were combined and concentrated to dryness affording 1-{5-[(2,6-dichlorophenyl) methoxy]pyrimidin-2-yl}-1,2,4-triazole, 3-carbonitrile (5.11 mg, 0.01 mmol, 2.84%) as a white powder (Purity: >95%). Further fractions pertaining to the amide (14-16) were combined and concentrated to dryness affording 9 mg of a white powder. Analysis of the powder showed a mixture of 3 compounds. The powder was re purified by automated flash chromatography (Combiflash 300+, Silica 4 g RediSep column) eluting with 0 to 100% Ethyl Acetate in Heptane Acetate in Heptane Fractions 13-21 were combined and concentrated to dryness affording 1-{5-[(2,6-dichlorophenyl) methoxy]pyrimidin-2-yl}-1,2,4-triazole-3-carboxamide (5.59 mg, 0.02 mmol, 2.95%) as a white powder (Purity: >95%).

### Example 192

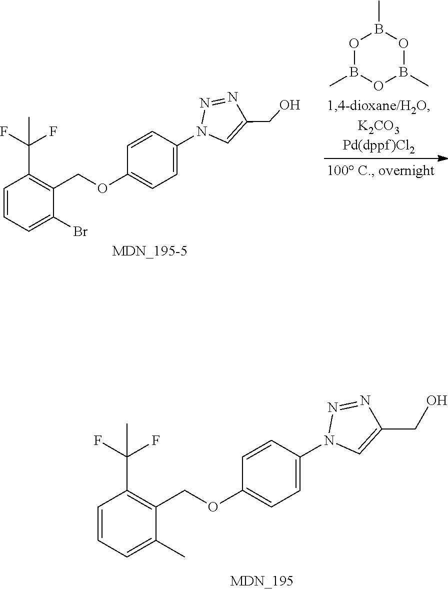

To a solution of MDN_195-5 (50 mg, 0.118 mmol) in 1,4-dioxane (1 mL) and H₂O (0.1 mL) were added 2,4,6-trimethyl-1,3,5,2,4,6-trioxatriborinane (0.5 mL, 0.590 mmol), K2CO3 (49 mg, 0.354 mmol) and Pd (dppf) C12 (17 mg, 0.024 mmol). The reaction was stirred under Ar atmosphere at 100oC overnight. The reaction mixture was diluted with H2O (20 mL) and extracted with EtOAc (20 mL×3). The organic layer was washed with brine (100 mL), dried over Na2SO4, filtered and concentrated in vacuo. The residue was purified by TLC (Ethyl Acetate: PE=1:1) to afford MDN_195 as a white solid (19 mg, 44.7% yield).

### Example 193

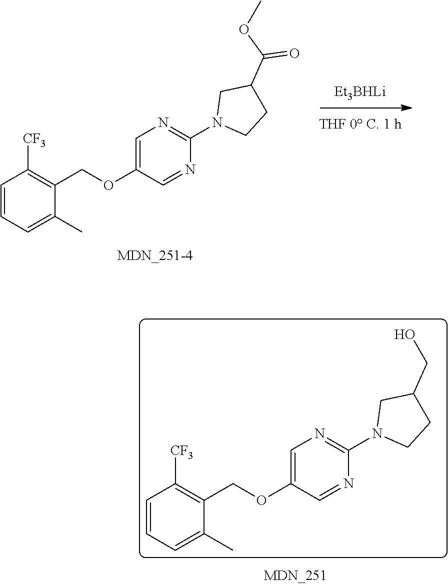

To a solution of MDN_251-4 (148 mg, 0.37 mmol) in THF (3 mL) was added Et3BHLi (1M in THF, 1.9 mL, 1.9 mmol) under ice cooling bath. The mixture was stirred under ice cooling bath for 2 h. The mixture was quenched with water, extracted with Ethyl Acetate (EA), dried over Na2SO4, concentrated, purified by pre-TLC (DCM:MeOH=10:1) to get the title compound MDN_251 as a colorless oil (68 mg, 50% yield).

### Example 195

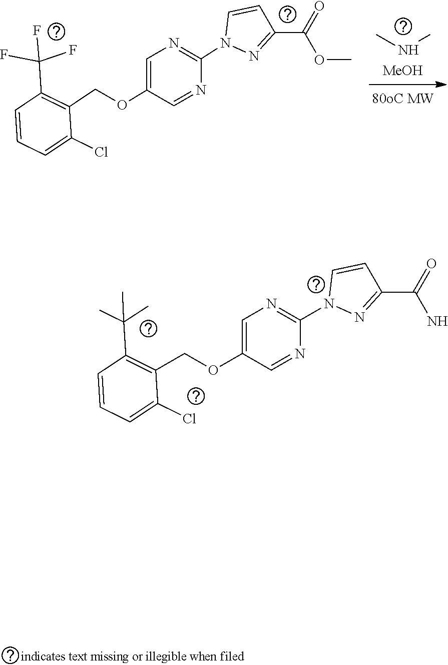

Methyl 1-(5-{[2-chloro-6-(trifluoromethyl)phenyl] methoxy}pyrimidin-2-yl) pyrazole-3-carboxylate (259 mg, 0.63 mmol, 1 eq) was taken up in ammonia (5.11 mL, 7M in, 35.77 mmol, 57 eq) (methanol) and added to a sealed tube, then heated to 80oC for 2 h on a timer behinde a blast shield. AM, rxn virtually complete by LC-MS. Rxn was conc in vacuo, then re-taken up in DCM and loaded onto isolute. purified by automated flash chromatography (Combiflash 300+, Silica 24 g RediSep column) eluting with 0 to 6% Methanol in Dichloromethane. F4 to F8 combined and conc in vacuo. The residue was triturated from ether, then filtered and dried under suction. A white solid was loaded into a submission vial and dried in vacuo over weekend at 40° C. to give 1-(5-{[2-chloro-6-(trifluoromethyl)phenyl] methoxy}pyrimidin-2-yl) pyrazole-3-carboxamide (88.9 mg, 0.22 mmol, 35.62%) as a white solid.

### Example 196

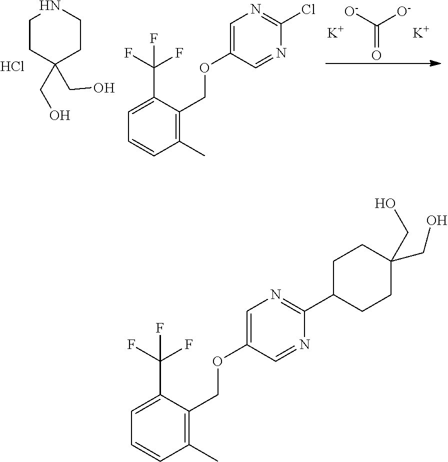

To a stirred solution of 2-chloro-5-{[2-methyl-6-(trifluoromethyl)phenyl] methoxy}pyrimidine (100 mg, 0.33 mmol, 1 eq) and POTASSIUM CARBONATE (136.98 mg, 0.99 mmol, 3 eq) in DMSO (2.5 mL) was added [4-(Hydroxymethyl) piperidin-4-yl]methanol HC11 (90.03 mg, 0.5 mmol, 1.5 eq). Reaction was sealed and heated to 100° C. for 96 h. Reaction was cooled to rt and filtered through a plug of celite. The mixture was directly purified by preparative HPLC automated flash chromatography (ISCO ACCQ HP125, Prep HPLC Column: Big GeminiNX C18 Dimensions: 30 mm×250 mm 5 μM; Sample: 2.50 ml from tube 1 RediSep column) eluting with 5 to 95% ACN/0.1% FA in Water/0.1% FA Desired fractions were combined and concentrated to dryness affording [4-(hydroxymethyl)-1-(5-{[2-methyl-6-(trifluoromethyl)phenyl] methoxy}pyrimidin-2-yl) piperidin-4-yl]methanol (73 mg, 0.18 mmol, 53.71%) as a white powder (Purity: >95%)

### Example 197

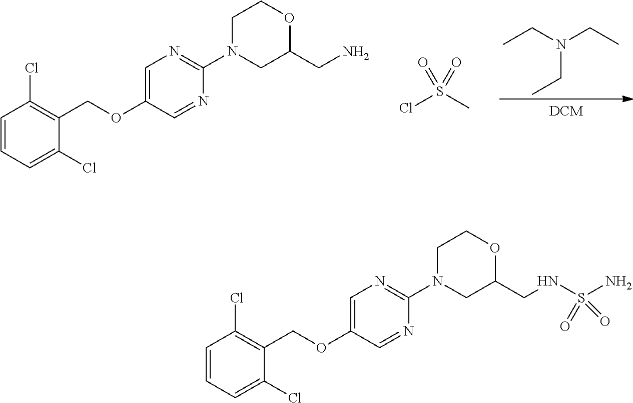

Methanesulfonyl chloride, VERC100459:17 (0.02 mL, 1.48 g/mL, 0.19 mmol, 1.5 eq) was added to a stirred solution of (4-{5-[(2,6-dichlorophenyl) methoxy]pyrimidin-2-yl}morpholin-2-yl) methanamine, 3549-RJP-029-002 (48 mg, 0.13 mmol, 1 eq) and TRIETHYLAMINE, VERC100628 (0.05 mL, 0.73 g/mL, 0.39 mmol, 3 eq) in DCM (3 mL). Ther eaction was stirred at rt for ca 1.5 h. The solvent was removed in vacuo then the resultant residue dissolved in DMSO, filtered and purified by preparative HPLC automated chromatography (ISCO ACCQ HP150, Prep HPLC Column: Gemini-NX C18 Dimensions: 21 mm×150 mm 5 μM; Sample: 1.60 ml from tube 1) eluting with 5 to 95% ACN/0.1% FA in Water/0.1% FA. Fractions 14 & 15 were combined then freeze dried. The sample was transferred to a submission vial using the minimum amount of DCM. The solvent was removed under a stream of nitrogen followed by in vacuo. Gave N-[(4-{5-[(2,6-dichlorophenyl) methoxy]pyrimidin-2-yl}morpholin-2-yl)methyl]methanesulfonamide (29.52 mg, 0.07 mmol, 50.76%) as a white foam (Purity: >95%).

### Example 198

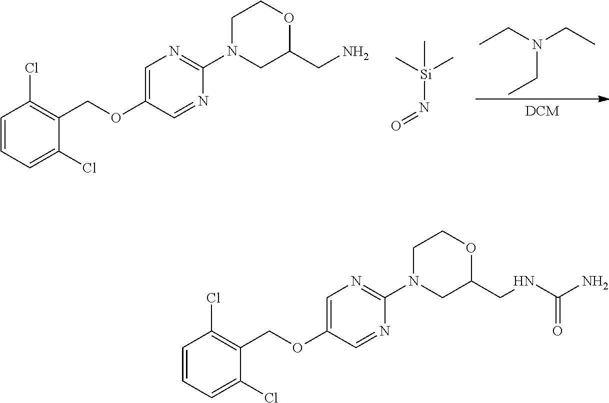

Trimethylsilyl isocyanate, 94% (0.03 mL, 0.85 g/mL, 0.19 mmol, 1.5 eq) was added to a stirred solution of (4-{5-[(2,6-dichlorophenyl) methoxy]pyrimidin-2-yl}morpholin-2-yl) methanamine (48 mg, 0.13 mmol, 1 eq) and TRIETHYLAMINE (0.05 mL, 0.73 g/mL, 0.39 mmol, 3 eq) in DCM (3 mL). The reaction was stirred at rt overnight. The solvent was removed in vacuo then the resultant residue dissolved in DMSO, filtered and purified by preparative HPLC automated chromatography (ISCO ACCQ HP150, Sample: 1.40 ml from tube 1; Prep HPLC Column: Gemini-NX C18 Dimensions: 21 mm×150 mm 5 M) eluting with 5 to 95% ACN/0.1% FA in Water/0.1% FA. Fractions 15 & 16 were combined then freeze dried. Gave (4-{5-[(2,6-dichlorophenyl) methoxy]pyrimidin-2-yl}morpholin-2-yl)methylurea (23.77 mg, 0.06 mmol, 44.35%) as a white solid (Purity: >95%).

### Example 199

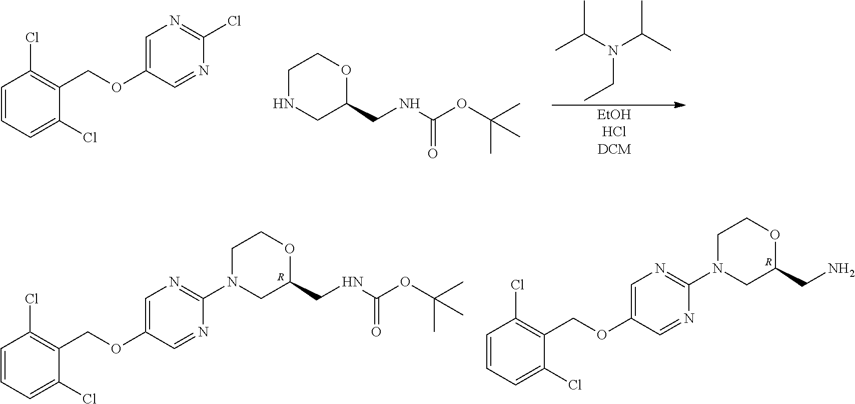

Nitrogen was bubbled through a solution of 2-chloro-5-[(2,6-dichlorophenyl) methoxy]pyrimidine (50 mg, 0.17 mmol, 1 eq), (R)-tert-Butyl (morpholin-2-ylmethyl) carbamate (56.02 mg, 0.26 mmol, 1.5 eq) and N,N-DIISOPROPYLETHYLAMINE (0.09 mL, 0.78 g/mL, 0.52 mmol, 3 eq) in ethanol (0.5 mL) in a microwave vial. The vial was microwaved at 130° C. for 2 h. LCMS shows mainly product with some starting material still present. The solvent was removed in vacuo then the residue dissolved in DCM (3 mL) and HYDROCHLORIC ACID (4N in 1,4-dioxane) (0.26 mL, 4M in, 1.04 mmol, 6 eq) added. The reaction was stirred at rt overnight. The solvent was removed in vacuo then the resultant residue dissolved in DMSO, filtered and purified by preparative HPLC automated chromatography (ISCO ACCQ HP150, Prep HPLC Column: Gemini-NX C18 Dimensions: 21 mm×150 mm 5 μM; Sample: 1.30 ml from tube 1) eluting with 5 to 95% ACN/0.1% FA in Water/0.1% FA. Fractions 4-7 were combined then freeze dried. Gave [(2S)-4-{5-[(2,6-dichlorophenyl) methoxy]pyrimidin-2-yl}morpholin-2-yl]methanamine (31.96 mg, 0.09 mmol, 50.12%) as a white solid (Purity: >95%) (FORMATE, 0.72 eq).

### Example 200

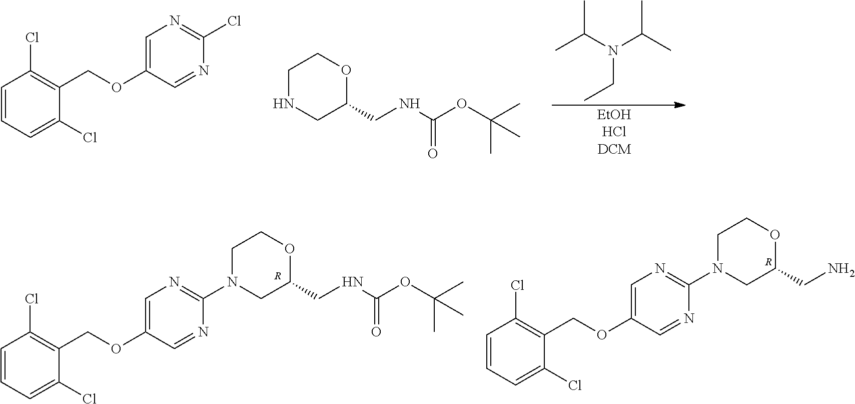

Nitrogen was bubbled through a solution of 2-chloro-5-[(2,6-dichlorophenyl) methoxy]pyrimidine (50 mg, 0.17 mmol, 1 eq), (S)-tert-Butyl (morpholin-2-ylmethyl) carbamate (56.02 mg, 0.26 mmol, 1.5 eq) and N,N-DIISOPROPYLETHYLAMINE (0.09 mL, 0.78 g/mL, 0.52 mmol, 3 eq) in ethanol (0.5 mL) in a microwave vial. The vial was microwaved at 130oC for 2 h. LCMS shows mainly product with some starting material still present. The solvent was removed in vacuo then the residue dissolved in DCM (3 mL) and HYDROCHLORIC ACID (4N in 1,4-dioxane) (0.26 mL, 4M in, 1.04 mmol, 6 eq) added. The reaction was stirred at rt overnight. The solvent was removed in vacuo then the resultant residue dissolved in DMSO, filtered and purified by preparative HPLC automated chromatography (ISCO ACCQ HP150, Sample: 1.30 ml from tube 1; Prep HPLC Column: Gemini-NX C18 Dimensions: 21 mm×150 mm 5 μM) eluting with 5 to 95% ACN/0.1% FA in Water/0.1% FA. Fractions 3-6 were combined then freeze dried. Gave [(2R)-4-{5-[(2,6-dichlorophenyl) methoxy]pyrimidin-2-yl}morpholin-2-yl]methanamine (26.55 mg, 0.07 mmol, 41.64%) as a white solid (3549-RJP-043-001, Purity: >95%) (FORMATE, 0.78 eq).

### Example 201

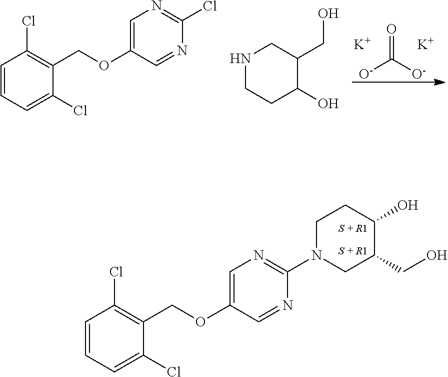

Nitrogen was bubbled through a DMSO (1 mL) solution of 2-chloro-5-[(2,6-dichlorophenyl) methoxy]pyrimidine (50 mg, 0.17 mmol, 1 eq), 3-(hydroxymethyl) piperidin-4-ol (22.65 mg, 0.17 mmol, 1 eq) and POTASSIUM CARBONATE (71.6 mg, 0.52 mmol, 3 eq) in a pressure tube. The tube was sealed then heated to 100oC, where it was maintained overnight. LCMS shows the reaction has gone to completion. The reaction was cooled to rt then filtered and purified by preparative HPLC automated chromatography (ISCO ACCQ HP150, Prep HPLC Column: Big Gemini-NX C18 Dimensions: 30 mm×250 mm 5 μM; Sample: 1.50 ml from tube 1) eluting with 5 to 95% ACN/0.1% FA in Water/0. 1% FA. Fractions 27-31 were combined then freeze dried. The sample was transferred to a submission vial using DCM then the solvent removed under a stream on nitrogen. The compound was dried under vacuum overnight at 40oC. No stereochemistry information was provided by the supplier for the starting piperidine however NMR of the product shows a single diasteroisomer (cis), chiral LCMS shows a mixture of enantiomers. Gave (3S,4S)-1-{5-[(2,6-dichlorophenyl) methoxy]pyrimidin-2-yl}-3-(hydroxymethyl) piperidin-4-ol (33.45 mg, 0.09 mmol, 50.41%) as a white solid (Purity: >95%).

### Example 202

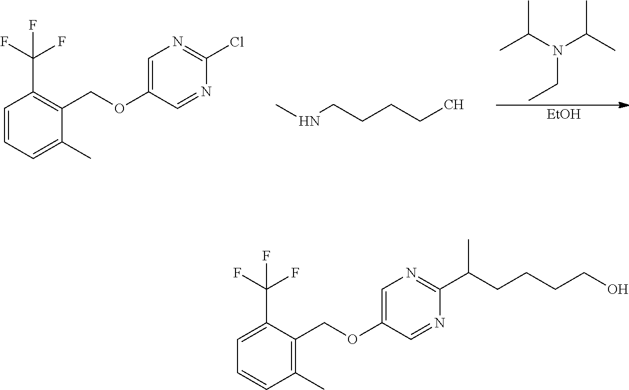

Nitrogen was bubbled through a solution of 2-chloro-5-{[2-methyl-6-(trifluoromethyl)phenyl] methoxy}pyrimidine (50 mg, 0.17 mmol, 1 eq), 4-(methylamino) butan-1-ol (25.56 mg, 0.25 mmol, 1.5 eq) and N,N-DIISOPROPYLETHYLAMINE (0.08 mL, 0.78 g/mL, 0.5 mmol, 3 eq) in ethanol (1 mL). The reaction was sealed then heated to 100oC, where it was maintained overnight. The reaction was cooled to rt then the solvent was removed in vacuo. The resultant residue dissolved in DMSO, filtered and purified by preparative HPLC automated chromatography (ISCO ACCQ HP150, Sample: 1.30 ml from tube 1; Prep HPLC Column: Gemini-NX C18 Dimensions: 21 mm×150 mm 5 M) eluting with 5 to 95% ACN/0.1% FA in Water/0.1% FA. Fractions 5-7 were combined then freeze dried. The resultant glass was transferred to a submission vial using DCM. The solvent was removed under a stream of nitrogen followed by vaccum then dried under vacuum at 40 oC overnight. Gave 4-[methyl (5-{[2-methyl-6-(trifluoromethyl)phenyl] methoxy}pyrimidin-2-yl) amino]butan-1-ol (31.18 mg, 0.08 mmol, 51.1%) as a colourless gum (Purity: >95%).

### Example 203

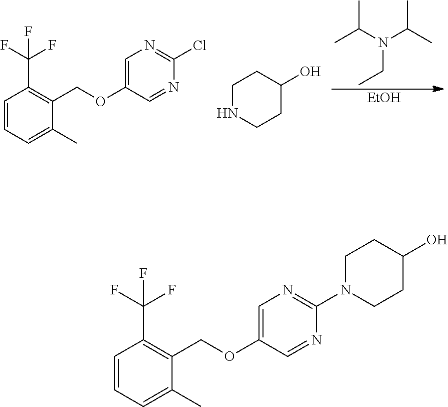

Nitrogen was bubbled through a solution of 2-chloro-5-{[2-methyl-6-(trifluoromethyl)phenyl] methoxy}pyrimidine (50 mg, 0.17 mmol, 1 eq), 4-HYDROXYPIPERIDINE (25.06 mg, 0 g/mL, 0.25 mmol, 1.5 eq) and N,N-DIISOPROPYLETHYLAMINE (0.08 mL, 0.78 g/mL, 0.5 mmol, 3 eq) in ethanol (1 mL). The reaction was sealed then heated to 100oC, where it was maintained overnight. The reaction was cooled to rt then the solvent was removed in vacuo. The resultant residue dissolved in DMSO, filtered and purified by preparative HPLC automated chromatography (ISCO ACCQ HP150, Sample: 1.30 ml from tube 1; Prep HPLC Column: Gemini-NX C18 Dimensions: 21 mm×150 mm 5 μM) eluting with 5 to 95% ACN/0.1% FA in Water/0.1% FA. Fractions 5 & 6 were combined then freeze dried. The resultant glass was transferred to a submission vial using DCM. The solvent was removed under a stream of nitrogen followed by in vacuo then dried under vacuum at 40oC overnight. Gave 1-(5-{[2-methyl-6-(trifluoromethyl)phenyl] methoxy}pyrimidin-2-yl) piperidin-4-ol (41.93 mg, 0.11 mmol, 69.09%) as a colourless gum (Purity: >95%).

### Example 204

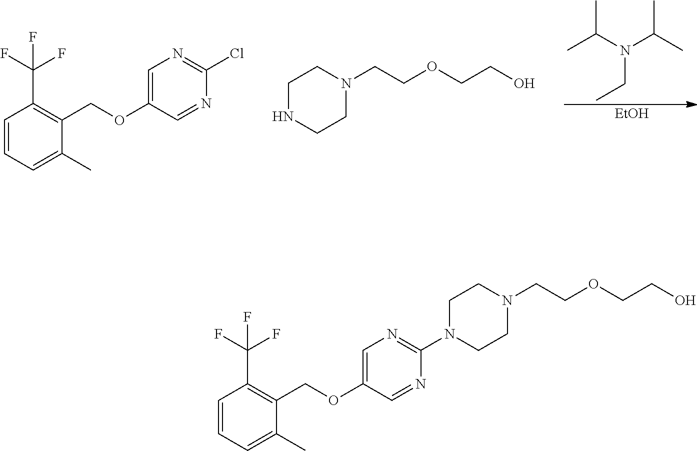

Nitrogen was bubbled through a solution of 2-chloro-5-{[2-methyl-6-(trifluoromethyl)phenyl] methoxy}pyrimidine (50 mg, 0.17 mmol, 1 eq), 1-[2-(2-HYDROXYETHOXY) ETHYL]PIPERAZINE (0.04 mL, 1.06 g/mL, 0.25 mmol, 1.5 eq) and N,NDIISOPROPYLETHYLAMINE (0.08 mL, 0.78 g/mL, 0.5 mmol, 3 eq) in ethanol (1 mL). The reaction was sealed then heated to 100° C., where it was maintained overnight. The reaction was cooled to rt then the solvent was removed in vacuo. The resultant residue dissolved in DMSO, filtered and purified by preparative HPLC automated chromatography (ISCO ACCQ HP150, Sample: 1.30 ml from tube 1; Prep HPLC Column: Gemini-NX C18 Dimensions: 21 mm×150 mm 5 μM) eluting with 5 to 95% ACN/0.1% FA in Water/0.1% FA. Fractions 3 & 4 were combined then freeze dried. The resultant glass was transferred to a submission vial using DCM. The solvent was removed under a stream of nitrogen followed by in vacuo then dried under vacuum at 40oC overnight. Gave 2-{2-[4-(5-{[2-methyl-6-(trifluoromethyl)phenyl] methoxy}pyrimidin-2-yl) piperazin-1-yl]ethoxy}ethanol (52.65 mg, 0.12 mmol, 72.36%) as a colourless gum (Purity: >95%) (FORMATE, 0.14 eq).

### Example 205

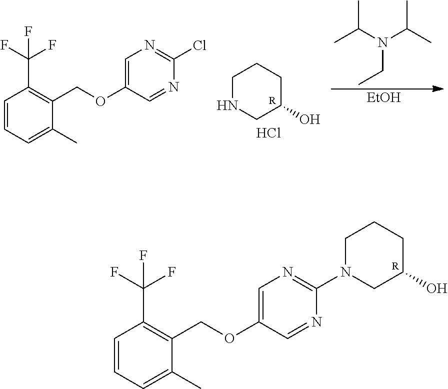

Nitrogen was bubbled through a solution of 2-chloro-5-{[2-methyl-6-(trifluoromethyl)phenyl] methoxy}pyrimidine (50 mg, 0.17 mmol, 1 eq), (S)-3-HYDROXYPIPERIDINE HYDROCHLORIDE, VERC112415:1 (34.1 mg, 0.25 mmol, 1.5 eq) and N,NDIISOPROPYLETHYLAMINE (0.14 mL, 0.78 g/mL, 0.83 mmol, 5 eq) in ethanol (0.5 mL). The reaction was sealed then microwaved at 130° C. for 2 h. The reaction was cooled to rt then the solvent was removed in vacuo. The resultant residue dissolved in DMSO, filtered and purified by preparative HPLC automated chromatography (ISCO ACCQ HP150, Sample: 1.30 ml from tube 1; Prep HPLC Column: Gemini-NX C18 Dimensions: 21 mm×150 mm 5 μM) eluting with 5 to 95% ACN/0.1% FA in Water/0.1% FA. Fractions 6 & 7 were combined then freeze dried. The resultant glass was transferred to a submission vial using DCM. The solvent was removed under a stream of nitrogen followed by the V10 then dried under vacuum at 40oC overnight. Gave (3S)-1-(5-{[2-methyl-6-(trifluoromethyl)phenyl] methoxy}pyrimidin-2-yl) piperidin-3-ol (33.5 mg, 0.09 mmol, 55.2%) as a colourless gum (Purity: >95%).

### Example 206

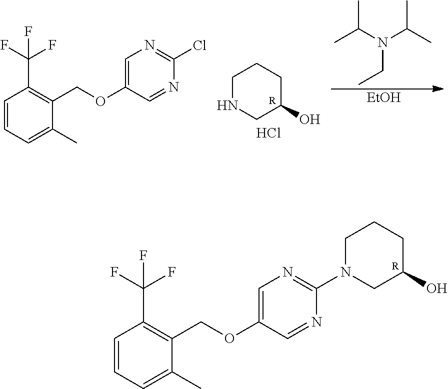

Nitrogen was bubbled through a solution of 2-chloro-5-{[2-methyl-6-(trifluoromethyl)phenyl] methoxy}pyrimidine (50 mg, 0.17 mmol, 1 eq), (R)-3-HYDROXYPIPERIDINE HYDROCHLORIDE (34.1 mg, 0.25 mmol, 1.5 eq) and N,NDIISOPROPYLETHYLAMINE (0.14 mL, 0.78 g/mL, 0.83 mmol, 5 eq) in ethanol (0.5 mL). The reaction was sealed then heated to 130oC for 2 h. The reaction was cooled to rt then the solvent was removed in vacuo. The resultant residue dissolved in DMSO, filtered and purified by preparative HPLC automated chromatography (ISCO ACCQ HP150, Sample: 1.30 ml from tube 1; Prep HPLC Column: Gemini-NX C18 Dimensions: 21 mm×150 mm 5 μM) eluting with 5 to 95% ACN/0.1% FA in Water/0.1% FA. Fractions 6 & 7 were combined then freeze dried. The resultant glass was transferred to a submission vial using DCM. The solvent was removed under a stream of nitrogen followed by in vacuo then dried under vacuum at 40oC overnight. Gave (3R)-1-(5-{[2-methyl-6-(trifluoromethyl)phenyl] methoxy}pyrimidin-2-yl) piperidin-3-ol (29.91 mg, 0.08 mmol, 49.29%) as a colourless gum (Purity: >95%).

### Example 207

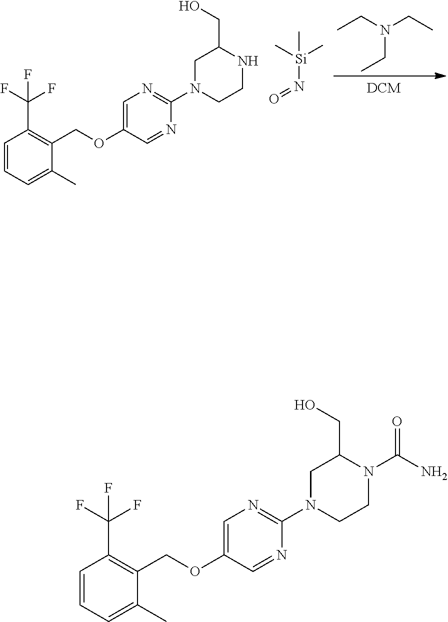

Trimethylsilyl isocyanate, 94% (0.04 mL, 0.85 g/mL, 0.31 mmol, 1.5 eq) was added to a stirred solution of [4-(5-{[2-methyl-6-(trifluoromethyl)phenyl] methoxy}pyrimidin-2-yl) piperazin-2-yl]methanol (78 mg, 0.2 mmol, 1 eq) and TRIETHYLAMINE (0.09 mL, 0.73 g/mL, 0.61 mmol, 3 eq) in DCM (5 mL). The solvent was removed in vacuo then the resultant residue dissolved in DMSO, filtered and purified by preparative HPLC automated chromatography (ISCO ACCQ HP150, Prep HPLC Column: Gemini-NX C18 Dimensions: 21 mm×150 mm 5 μM; Sample: 1.40 ml from tube 1) eluting with 5 to 95% ACN/0.1% FA in Water/0.1% FA. Fractions 5 was freeze dried. Gave 2-(hydroxymethyl)-4-(5-{[2-methyl-6-(trifluoromethyl)phenyl] methoxy}pyrimidin-2-yl) piperazine-1-carboxamide (24.8 mg, 0.06 mmol, 28.58%) as a white solid (Purity: >95%).

### Example 208

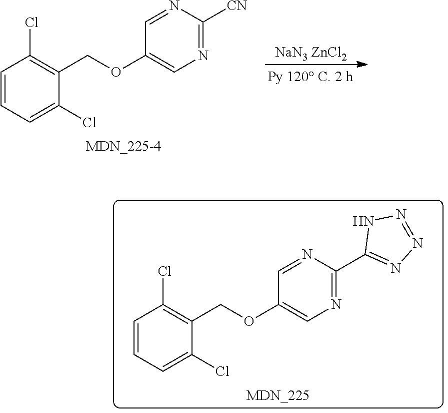 [SMILES: N#Cc1ncc(OCc2c(Cl)cccc2Cl)cn1 ; Clc1cccc(Cl)c1COc1cnc(-c2nnn[nH]2)nc1 ; "NaN3 ZnCl2"]

A mixture of MDN_225-4 (50 mg, 0.178 mmol), NaN3 (29 mg, 0.446 mmol) and ZnC12 (37 mg, 0.267 mmol) in Py (2 mL) was stirred at 120oC for 2 h. The mixture was quenched with water, filtered, washed with water, 1N HCl, water, dried in vacuo to get a white solid. The solid was washed with DCM, filtered, dried in vacuo to get the title compound MDN_251-3 as a white solid (10 mg, 18% yield).

### Example 209

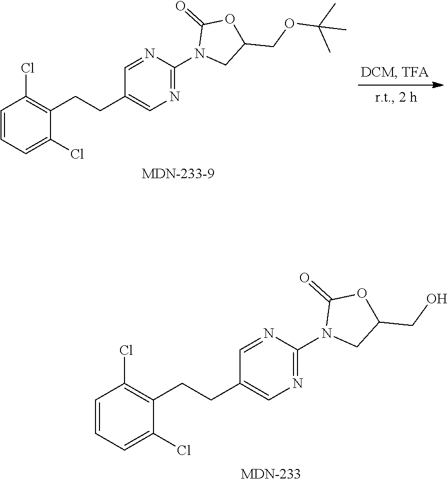 [SMILES: CC(C)(C)OCC1CN(c2ncc(CCc3c(Cl)cccc3Cl)cn2)C(=O)O1 ; O=C1OC(CO)CN1c1ncc(CCc2c(Cl)cccc2Cl)cn1]

To a mixture of MDN_233-9 (0.045 g, 0.106 mmol) in DCM (2.5 mL) was added TFA (0.5 mL). The reaction was stirred at r.t. for 2 h. The mixture was neutralized with aqueous NaHCO3 to pH 7˜8 and extracted with DCM (30 mL×3). The organic phase was dried over Na2SO4, filtered and concentrated. The crude product was purified by silica gel column (EA/PE=0%˜50%) to give the title compound as a yellow solid (15 mg, 38.0% yield).

### Example 210

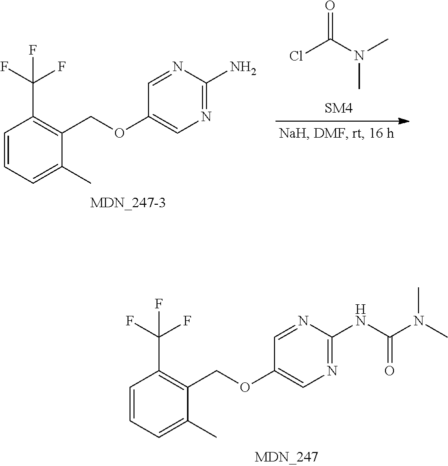

To a solution of MDN_247-3 (300 mg, 1.06 mmol) in DMF (2 mL) was added NaH (127 mg, 3.18 mmol) at 0° C. The mixture was stirred at rt for 1 h. Dimethylcarbamic chloride (228 mg, 2.12 mmol) was added. The reaction was stirred at rt for 16 h. The reaction mixture was diluted with H₂O and extracted with EA. The organic layer was dried over

Na2SO4, filtered and concentrated. The residue was purified by prep-HPLC (TFA) to obtain MDN_247 (15 mg, 4.0% yield, as a white solid).

### Example 211

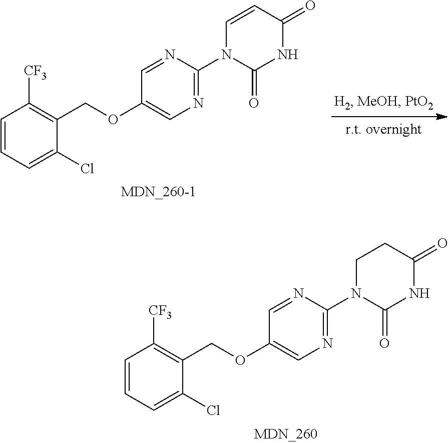 [SMILES: O=c1ccn(-c2ncc(OCc3c(Cl)cccc3C(F)(F)F)cn2)c(=O)[nH]1 ; O=C1CCN(c2ncc(OCc3c(Cl)cccc3C(F)(F)F)cn2)C(=O)N1]

To a solution of MDN_260-1 (50 mg, 0.125 mmol) in MeOH (5 mL) was added Pd/C (5 mg). The reaction was stirred at r.t. under H₂ atmosphere overnight. The reaction mixture was filtered through a pad of Celite and rinsed with MeOH (5 mL×3). The filtrate was concentrated in vacuo and the residue was purified by TLC (EA: PE=1:1) to afford MDN_260 as a white solid (5 mg, 9.2% yield).

### Example 212

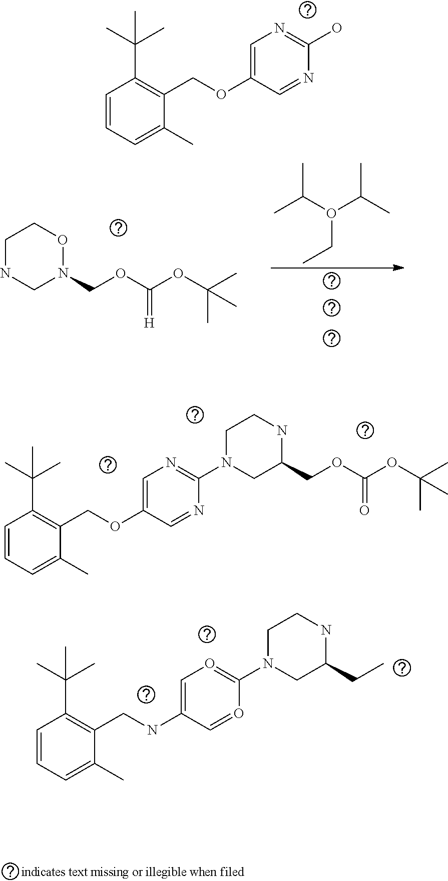

Nitrogen was bubbled through a solution of 2-chloro-5-{[2-methyl-6-(trifluoromethyl)phenyl] methoxy}pyrimidine (100 mg, 0.33 mmol, 1 eq), (R)-tert-Butyl (morpholin-2-ylmethyl) carbamate (107.18 mg, 0.5 mmol, 1.5 eq) and N,NDIISOPROPYLETHYLAMINE (0.16 mL, 0.78 g/mL, 0.99 mmol, 3 eq) in ethanol (2 mL). The reaction was sealed then heated to 100° C., where it was maintained overnight. LCMS shows mainly product with some starting material still present. The solvent was removed in vacuo then the resultant residue dissolved in DCM (3 mL) and HYDROCHLORIC ACID (4N in 1,4-dioxane) (0.5 mL, 4M in, 1.98 mmol, 6 eq) added. The reaction was stirred at rt for ca 5 h. The solvent was removed in vacuo then the resultant residue dissolved in MeOH and applied an scx-2 cartridge (2 g pre-washed with MeOH). The column was washed with MeOH then compound eluted with 3.5N NH₃ in MeOH. The solvent was removed in vacuo. Gave [(2S)-4-(5-{[2-methyl-6-(trifluoromethyl)phenyl] methoxy}pyrimidin-2-yl) morpholin-2-yl]methanamine (107 mg, 0.28 mmol, 84.7%) as a beige gum and further purified by preparative HPLC automated chromatography (ISCO ACCQ HP150, Prep HPLC Column: Gemini-NX C18 Dimensions: 21 mm×150 mm 5 μM; Sample: 1.30 ml from tube 1) eluting with 5 to 95% ACN/0.1% FA in Water/0.1% FA to give [(2S)-4-(5-{[2-methyl-6-(trifluoromethyl)phenyl] methoxy}pyrimidin-2-yl) morpholin-2-yl]methanamine (27.94 mg, 0.07 mmol, 22.12%) as a white solid (Purity: >95%) (FORMATE, 0.79 eq).

### Example 213

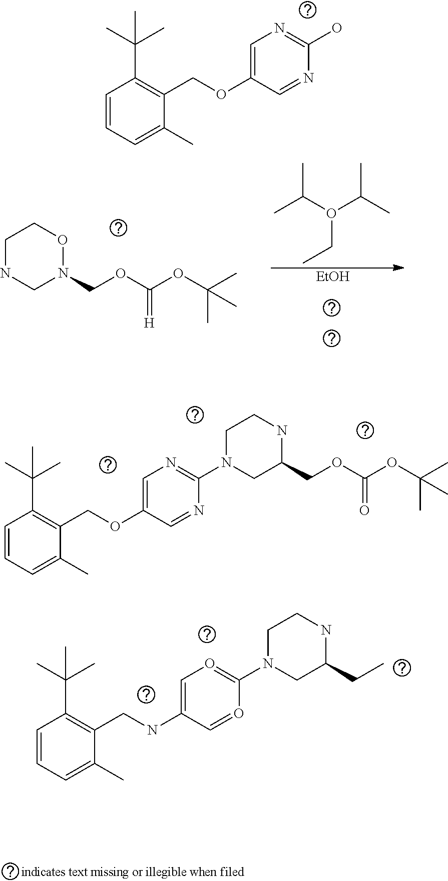

Nitrogen was bubbled through a solution of 2-chloro-5-{[2-methyl-6-(trifluoromethyl)phenyl] methoxy}pyrimidine (100 mg, 0.33 mmol, 1 eq), (S)-tert-Butyl (morpholin-2-ylmethyl) carbamate (107.18 mg, 0.5 mmol, 1.5 eq) and N,N-DIISOPROPYLETHYLAMINE (0.16 mL, 0.78 g/mL, 0.99 mmol, 3 eq) in ethanol (2 mL). The reaction was sealed then heated to 100° C., where it was maintained overnight. LCMS shows mainly product with some starting material still present. The solvent was removed in vacuo then the resultant residue dissolved in DCM (3 mL) and HYDROCHLORIC ACID (4N in 1,4-dioxane) (0.5 mL, 4M in, 1.98 mmol, 6 eq) added. The reaction was stirred at rt for ca 5 h. The solvent was removed in vacuo then the resultant residue dissolved in MeOH and applied an scx-2 cartridge (2 g pre-washed with MeOH). The column was washed with MeOH then compound eluted with 3.5N NH₃ in MeOH. The solvent was removed in vacuo. Gave [(2R)-4-(5-{[2-methyl-6-(trifluoromethyl)phenyl] methoxy}pyrimidin-2-yl) morpholin-2-yl]methanamine (93 mg, 0.24 mmol, 73.62%) as a beige gum and was purified by preparative HPLC automated chromatography (ISCO ACCQ HP150, Prep HPLC Column: Gemini-NX C18 Dimensions: 21 mm×150 mm 5 μM; Sample: 1.30 ml from tube 1) eluting with 5 to 95% ACN/0.1% FA in Water/0.1% FA. Fractions 9 & 10 were combined then freeze dried. Gave [(2R)-4-(5-{[2-methyl-6-(trifluoromethyl)phenyl] methoxy}pyrimidin-2-yl) morpholin-2-yl]methanamine (23.54 mg, 0.06 mmol, 18.63%) as a white solid (,Purity: >95%) (FORMATE, 0.6 eq).

### Example 214

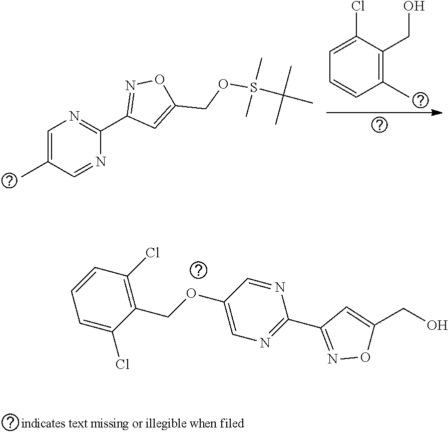

To a stirred colourless solution of 2,6-Dichlorobenzyl Alcohol (7.53 mg, 0.04 mmol, 1.05 eq) in THF (0.1 mL) under nitrogen was added portion-wise Sodium Hydride (1.86 mg (60%), 0.05 mmol, 1.15 eq) and the white suspension stirred for 10 min. To which a solution of 5-bromo-2-(5-{[(tert-butyldimethylsilyl)oxy] methyl}-1,2-oxazol-3-yl)pyrimidine (15 mg, 0.04 mmol, 1 eq) in THF (0.2 mL) was added dropwise and the yellow reaction mixture stirred at room temperature for 4 h. The reaction mixture had dried under nitrogen and was diluted with THF (0.3 mL) and the red reaction mixture stirred at 21° C. for 4 Days, LCMS analysis showed only 7% conversion of starting material to desired product. 2,6-Dichlorobenzyl Alcohol (22.6 mg, 0.12 mmol, 3.15 eq) and Sodium Hydride (5.6 mg (60%), 0.15 mmol, 3.45 eq) in a new vial under nitrogen was added THF (0.3 mL) and the white suspension stirred for 10 mins. This alkoxide mixture was then added dropwise to the previous reaction mixture and stirred at room temperature for 4 days. The reaction had run dry. To the reaction mixture was added THF (0.2 mL). The reaction was quenched with methanol (1 mL), diluted with water (20 mL), extracted with dichloromethane (15 mL), then dichloromethane:methanol (4:1, 15 mL), the combined organics were washed with brine (20 mL), passed through a hydrophobic frit and concentrated under reduced pressure to give a pale yellow oil which solidified on standing (47 mg). The crude mixture of TBDMS protected product were diluted with methanol, transferred to a vial, concentrated to give 26.5 mg, to which was added under nitrogen HCl (4M in Dioxane) (0.51 mL (4M), 2.03 mmol, 50 eq) The reaction mixture was transferred to a round bottomed flask with dichloromethane, concentrated under reduced pressure, diluted with DMSO and purified by preparative HPLC automated chromatography (ISCO ACCQ HP125, Prep HPLC Column: Gemini-NX C18 Dimensions: 21 mm×150 mm 5 μM; Sample: 1.60 ml from tube 1) eluting with 5 to 95% ACN/0.1% FA in Water/0.1% FA 95% ACN/0.1% FA in Water/0.1% FA. Fractions containing the desired product (were lyophilised to give (3-{5-[(2,6-dichlorophenyl) methoxy]pyrimidin-2-yl}-1,2-oxazol-5-yl) methanol (1.2 mg, 3.41 umol, 8.41%) as a white solid (Purity: >95%).

### Example 215

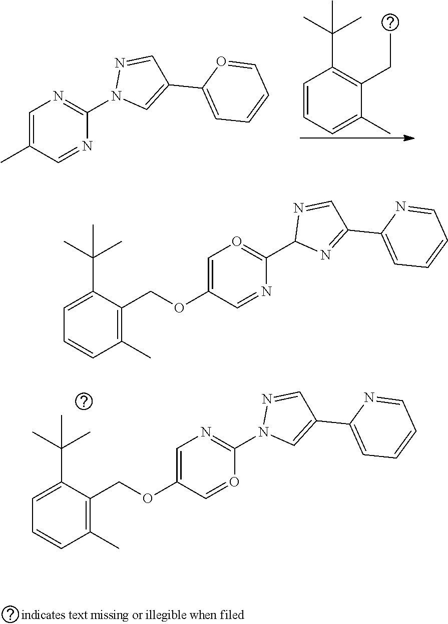

To a solution of the above starting material (108 mg, 0.45 mmol, 1 eq) in DMF (2 mL) was added 2-METHYL-6-TRIFLUOROMETHYLBENZYL BROMIDE (68.54 μL, 1.66 g/cm³, 0.45 mmol, 1 eq) followed by potassium carbonate (124.27 mg, 0.9 mmol, 2 eq) potassium carbonate (124.27 mg, 0.9 mmol, 2 eq) Work-up: The reaction mixture was filtered and purified by preparative HPLC automated chromatography (ISCO ACCQ HP125, Prep HPLC Column: Big Gemini-NX C18 Dimensions: 30 mm×250 mm 5 μM; Sample: 2.50 ml from tube 1) eluting with 5 to 95% ACN/0.1% FA in Water/0.1% FA. The desired fractoions were isolated to give 5-{[2-methyl-6-(trifluoromethyl)phenyl] methoxy}-2-[4-(pyridin-2-yl)-1,2,3-triazol-2-yl]pyrimidine (27 mg, 0.07 mmol, 14.56%) as a white solid (Purity: >95%).

### Example 216

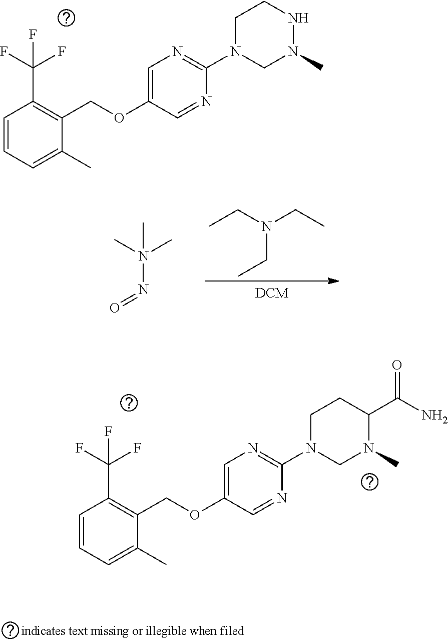

A solution of 5-{[2-methyl-6-(trifluoromethyl)phenyl] methoxy}-2-[(3S)-3-methylpiperazin-1-yl]pyrimidine (34 mg, 0.09 mmol, 1 eq), Trimethylsilyl isocyanate, 94%, (0.02 mL, 0.85 g/mL, 0.14 mmol, 1.5 eq) and TRIETHYLAMINE (0.04 mL, 0.73 g/mL, 0.28 mmol, 3 eq) in DCM (2 mL) was stirred at rt overnight. The solvent was removed in vacuo then the resultant residue dissolved in DMSO, filtered and purified by preparative HPLC automated chromatography (ISCO ACCQ HP150, Sample: 1.50 ml from tube 1; Prep HPLC Column: Gemini-NX C18 Dimensions: 21 mm×150 mm 5 μM) eluting with 19 to 95% ACN/0.1% FA in Water/0.1% FA. Fractions 12 & 13 were combined then freeze dried. Gave (2S)-2-methyl-4-(5-{[2-methyl-6-(trifluoromethyl)phenyl] methoxy}pyrimidin-2-yl) piperazine-1-carboxamide (19.27 mg, 0.05 mmol, 50.72%) as a white solid (Purity: >95%).

### Example 217

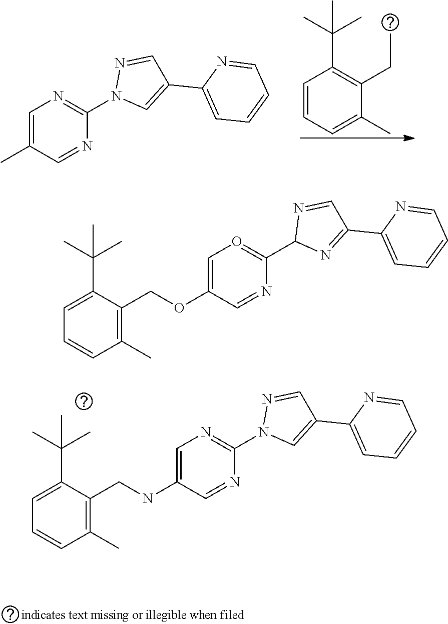

To a solution of the starting alcohol above (108 mg, 0.45 mmol, 1 eq) in DMF (2 mL) was added 2-METHYL-6-TRIFLUOROMETHYLBENZYL BROMIDE (68.54 μL, 1.66 g/cm³, 0.45 mmol, 1 eq) followed by potassium carbonate (124.27 mg, 0.9 mmol, 2 eq) potassium carbonate (124.27 mg, 0.9 mmol, 2 eq) The reaction mixture was filtered and purified by preparative HPLC automated chromatography (ISCO ACCQ HP125, Prep HPLC Column: Big Gemini-NX C18 Dimensions: 30 mm×250 mm 5 μM; Sample: 2.50 ml from tube 1) eluting with 5 to 95% ACN/0.1% FA in Water/0.1% FA. The desired fractions were combined and concentrated to give 5-{[2-methyl-6-(trifluoromethyl)phenyl] methoxy}-2-[4-(pyridin-2-yl)-1,2,3-triazol-2-yl]pyrimidine (27 mg, 0.07 mmol, 14.56%) as a white solid (Purity: >95%).

### Example 218

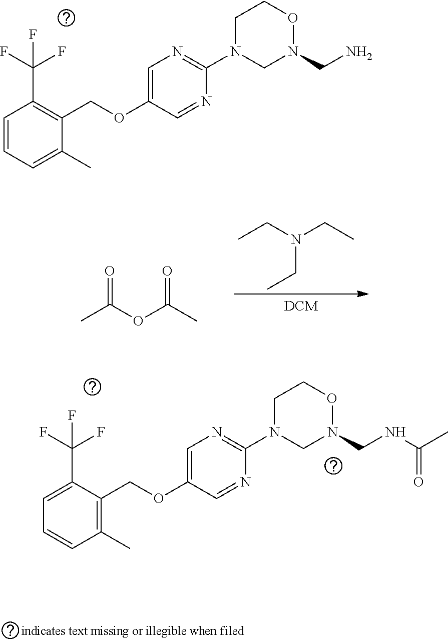

A solution of [(2S)-4-(5-{[2-methyl-6-(trifluoromethyl)phenyl] methoxy}pyrimidin-2-yl) morpholin-2-yl]methanamine (40 mg, 0.1 mmol, 1 eq), Acetic anhydride, 99+% (0.01 mL, 1.08 g/mL, 0.16 mmol, 1.5 eq) and TRIETHYLAMINE (0.04 mL, 0.73 g/mL, 0.31 mmol, 3 eq) in DCM (3 mL) was stirred at rt for ca 4 h. The reaction was stood at rt overnight. The solvent was removed in vacuo then the resultant residue dissolved in DMSO, filtered and purified by preparative HPLC automated chromatography (ISCO ACCQ HP150, Sample: 1.30 ml from tube 1; Prep HPLC Column: Gemini-NX C18 Dimensions: 21 mm×150 mm 5 μM) eluting with 19 to 95% ACN/0.1% FA in Water/0.1% FA. Fractions 1 & 2 were combined then freeze dried. Gave N-{[(2S)-4-(5-{[2-methyl-6-(trifluoromethyl)phenyl] methoxy}pyrimidin-2-yl) morpholin-2-yl] methyl}acetamide (15.41 mg, 0.04 mmol, 34.71%) as a white solid.

### Example 219

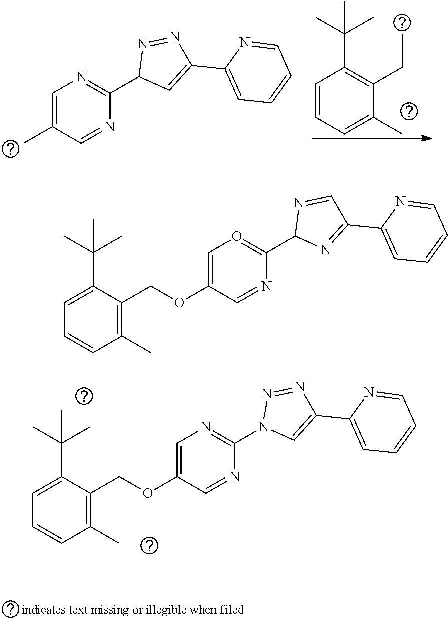

To a solution of the starting alcohol above (108 mg, 0.45 mmol, 1.08 eq) DMF (2 mL) was added 2-Chloro-6-(trifluoromethyl)benzyl bromide (68.41 μL, 1.66 g/cm³, 0.42 mmol, 1 eq) followed by potassium carbonate (114.99 mg, 0.83 mmol, 2 eq). The reaction mixture was heated at RT overnight. The reaction mixture was filtered and purified by preparative HPLC automated chromatography (ISCO ACCQ HP125, Sample: 2.70 ml from tube 1; Prep HPLC Column: Big Gemini-NX C18 Dimensions: 30 mm×250 mm 5 μM) eluting with 5 to 95% ACN/0.1% FA in Water/0. 1% FA. Fractions 46-48 5-{[2-chloro-6-(trifluoromethyl)phenyl] methoxy}-2-[4-(pyridin-2-yl)-1,2,3-triazol-2-yl]pyrimidine (30 mg, 0.07 mmol, 15.42%) white solid (Purity: >95%) Fractions 51-54 5-{[2-chloro-6-(trifluoromethyl)phenyl] methoxy}-2-[4-(pyridin-2-yl)-1,2,3-triazol-1-yl]pyrimidine (29 mg, 0.07 mmol, 14.9%) white solid (Purity: 90-95%).

### Example 220

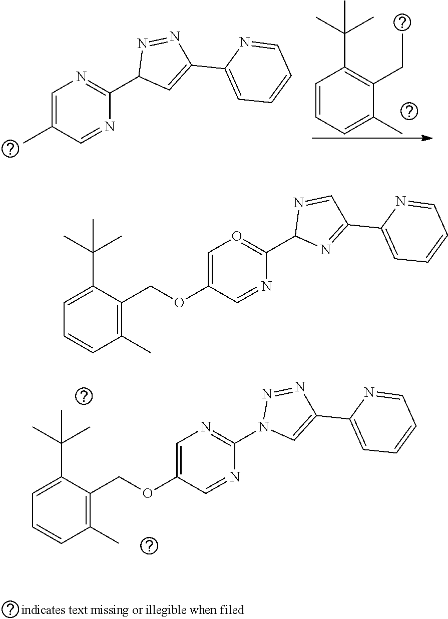

To a solution of the starting alcohol above (108 mg, 0.45 mmol, 1.08 eq) DMF (2 mL) was added 2-Chloro-6-(trifluoromethyl)benzyl bromide (68.41 μL, 1.66 g/cm³, 0.42 mmol, 1 eq) followed by potassium carbonate (114.99 mg, 0.83 mmol, 2 eq). The reaction mixture was heated at RT overnight. The reaction mixture was filtered and purified by preparative HPLC automated chromatography (ISCO ACCQ HP125, Sample: 2.70 ml from tube 1; Prep HPLC Column: Big Gemini-NX C18 Dimensions: 30 mm×250 mm 5 μM) eluting with 5 to 95% ACN/0.1% FA in Water/0. 1% FA

Fractions 46-48 5-{[2-chloro-6-(trifluoromethyl)phenyl] methoxy}-2-[4-(pyridin-2-yl)-1,2,3-triazol-2-yl]pyrimidine (30 mg, 0.07 mmol, 15.42%) white solid. Fractions 51-54 5-{[2-chloro-6-(trifluoromethyl)phenyl] methoxy}-2-[4-(pyridin-2-yl)-1,2,3-triazol-1-yl]pyrimidine (29 mg, 0.07 mmol, 14.9%) white solid (Purity: 90-95%) white solid.

### Example 221

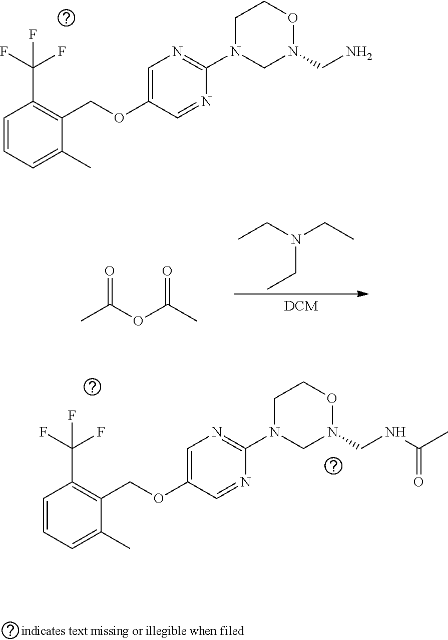

A solution of [(2R)-4-(5-{[2-methyl-6-(trifluoromethyl)phenyl] methoxy}pyrimidin-2-yl) morpholin-2-yl]methanamine (40 mg, 0.1 mmol, 1 eq), Acetic anhydride, 99+% (0.01 mL, 1.08 g/mL, 0.16 mmol, 1.5 eq) and TRIETHYLAMINE, (0.04 mL, 0.73 g/mL, 0.31 mmol, 3 eq) in DCM (3 mL) was stirred at rt for ca 4 h. LCMS shows the reaction has gone to completion. The reaction was stood at rt overnight. The solvent was removed in vacuo then the resultant residue dissolved in DMSO, filtered and purified by preparative HPLC automated chromatography (ISCO ACCQ HP150, Sample: 1.50 ml from tube 1; Prep HPLC Column: Gemini-NX C18 Dimensions: 21 mm×150 mm 5 μM) eluting with 19 to 95% ACN/0.1% FA in Water/0.1% FA. Fractions 8 & 9 were combined then freeze dried. Gave N-{[(2R)-4-(5-{[2-methyl-6-(trifluoromethyl)phenyl] methoxy}pyrimidin-2-yl) morpholin-2-yl]methyl}acetamide (17.84 mg, 0.04 mmol, 40.18%) as a white solid (Purity: >95%).

### Example 222

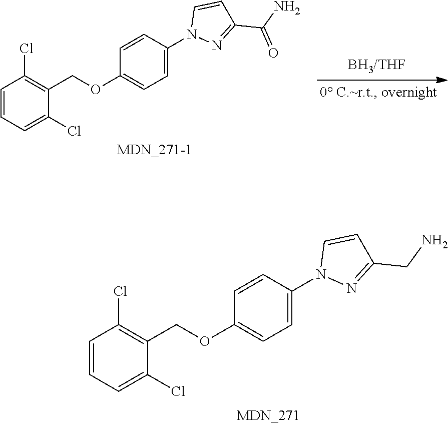 [SMILES: NC(=O)c1ccn(-c2ccc(OCc3c(Cl)cccc3Cl)cc2)n1 ; NCc1ccn(-c2ccc(OCc3c(Cl)cccc3Cl)cc2)n1]

To a solution of MDN_271-1 (80 mg, 0.220 mmol) in THF (2 mL) was added BH3 in THF (2.20 mL, 2.21 mmol) at 0oC. The reaction was allowed to warm to room temperature and stirred overnight. The reaction mixture was concentrated in vacuo. The crude product was purified by TLC (DCM:MeOH=10:1) to afford the title compound as a white solid (10.4 mg, 13.5% yield).

### Example 223

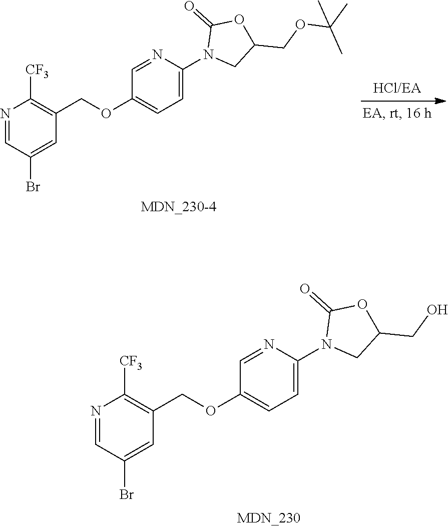 [SMILES: CC(C)(C)OCC1CN(c2ccc(OCc3cc(Br)cnc3C(F)(F)F)cn2)C(=O)O1 ; O=C1OC(CO)CN1c1ccc(OCc2cc(Br)cnc2C(F)(F)F)cn1]

To a solution of MDN_230-4 (30 mg, 0.060 mmol) in EA (1 mL) was added HCl/EA (1 mL). The reaction was stirred at rt for 16 h. The mixture was concentrated and the residue was purified by prep-HPLC (TFA) to obtain MDN_230 (15 mg, 55.8% yield, as a white solid).

### Example 224

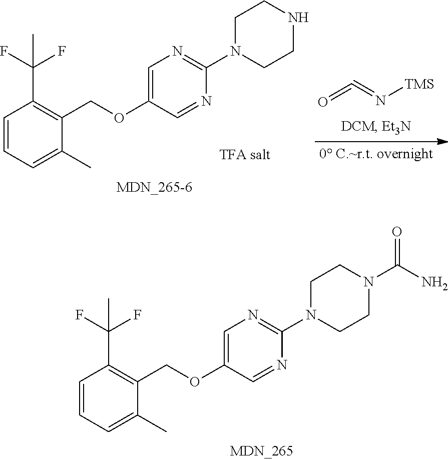 [SMILES: Cc1cccc(C(C)(F)F)c1COc1cnc(N2CCNCC2)nc1 ; C[Si](C)(C)N=C=O ; Cc1cccc(C(C)(F)F)c1COc1cnc(N2CCN(C(N)=O)CC2)nc1]

To a solution of MDN_265-6 (52 mg, 0.150 mmol) and Et₃N (46 mg, 0.450 mmol) in DCM (2 mL) was added isocyanatotrimethylsilane (26 mg, 0.225 mmol) at 0oC. The reaction was stirred at r.t. overnight. The reaction mixture was diluted with H₂O (10 mL) and extracted with DCM (20 mL×2). The organic layer was dried over Na2SO4, filtered and concentrated in vacuo. The residue was purified by TLC (DCM:MeOH=10:1) to afford MDN_265 as a off-white solid (15 mg, 25.7% yield).

### Example 225

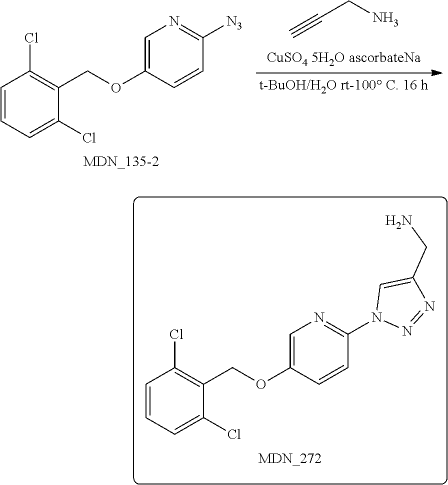

A mixture of MDN_135-2 (200 mg, 0.678 mmol), prop-2-yn-1-amine (42 mg, 0.745 mmol), CuSO4 5H₂O (170 mg, 0.678 mmol) and ascorbateNa (68 mg, 0.339 mmol) in t-BuOH/H₂O (4.0 mL/1.0 mL) was stirred at 100oC for 2 h. The mixture was filtered, concentrated, diluted with water, extracted with DCM, dried over Na2SO4, purified by reversed phase column (H₂O: MeCN) to give the title compound MDN_251-4 as a white solid (12 mg, 5% yield).

### Example 226

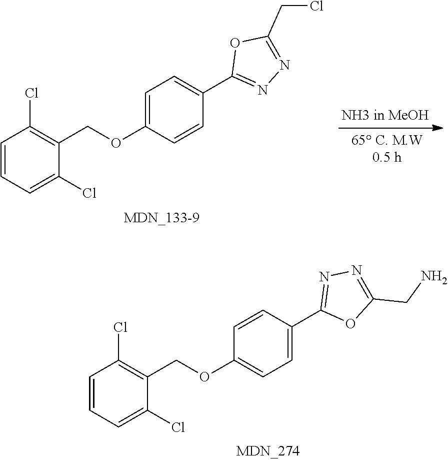 [SMILES: ClCc1nnc(-c2ccc(OCc3c(Cl)cccc3Cl)cc2)o1 ; NCc1nnc(-c2ccc(OCc3c(Cl)cccc3Cl)cc2)o1]

A solution of MDN_133-9 (150 mg, 0.41 mmol) in NH₃ in MeOH (7M, 10 mL, 70 mmol) was stirred at 65oC in microwave for 30 min. The mixture was concentrated, quenched with H2O, extracted with DCM, dried over Na2SO4, purified by silica gel column (PE: EA=1:1) to give the title compound MDN_255-11 as a white solid (80 mg, 57% yield).

### Example 227

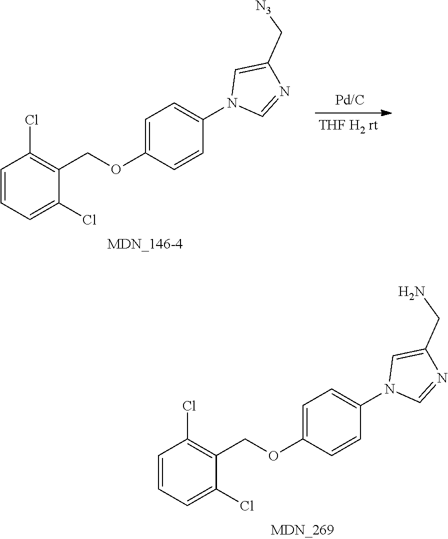 [SMILES: [N-]=[N+]=NCc1cn(-c2ccc(OCc3c(Cl)cccc3Cl)cc2)cn1 ; NCc1cn(-c2ccc(OCc3c(Cl)cccc3Cl)cc2)cn1]

To a solution of MDN_146-4 (0.174 g, 0.465 mmol) and in MeOH (3 mL) was added Pd/C (10%, 0.020 g) at rt. The mixture was stirred at rt for 4 h under H₂. The mixture was filtered, concentrated, purified by Pre-TLC (DCM:MeOH=7:1) to give the title compound MDN_269 as a yellow oil (0.025 g, 16% yield).

### Example 228

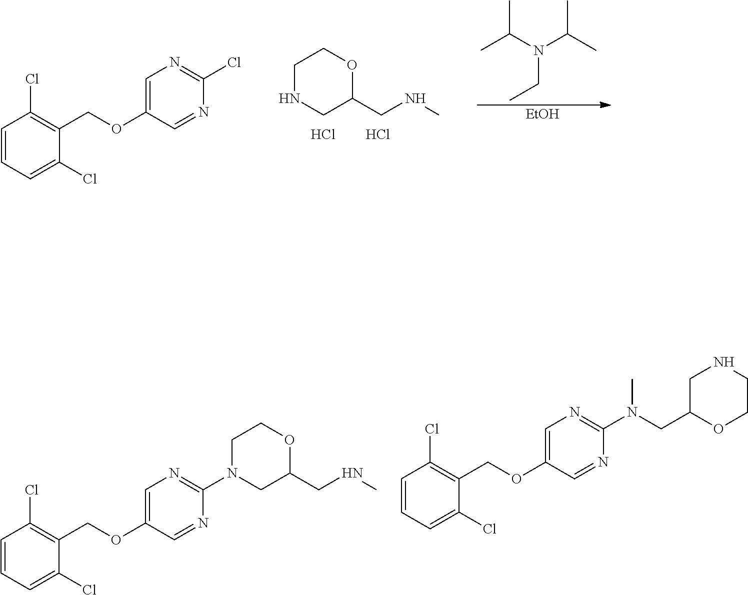

Nitrogen was bubbled through a solution of 2-chloro-5-[(2,6-dichlorophenyl) methoxy]pyrimidine (50 mg, 0.17 mmol, 1 eq), Methyl-morpholin-2-ylmethyl-amine dihydrochloride (52.61 mg, 0.26 mmol, 1.5 eq) and N,N-DIISOPROPYLETHYLAMINE (0.14 mL, 0.78 g/mL, 0.86 mmol, 5 eq) in ethanol (0.5 mL). The vial was capped then microwaved at 130° C. for 2 h. The solvent was removed in vacuo then the resultant residue dissolved in DMSO, filtered and purified by preparative HPLC automated chromatography (ISCO ACCQ HP150, Prep HPLC Column: Gemini-NX C18 Dimensions: 21 mm×150 mm 5 μM; Sample: 1.50 ml from tube 1) eluting with 5 to 95% ACN/0.1% ammonia in Water/0.1% ammonia. Fractions 2 & 3 were combined then freeze dried. The resultant gum was transferred to a submission vial using DCM then the solvent removed in under a stream of nitrogen. The sample was dried overnight under vacuum at 40° C. Gave 5-[(2,6-dichlorophenyl) methoxy]-Nmethyl-N-(morpholin-2-ylmethyl)pyrimidin-2-amine (6.67 mg, 0.02 mmol, 10.08%) as a colourless gum (Purity: >95%). Fractions 6+9-13 were combined then freeze dried. The resultant gum was transferred to a submission vial using DCM then the solvent removed in under a stream of nitrogen. The sample was dried overnight under vacuum at 40oC. Gave [(4-{5-[(2,6-dichlorophenyl) 1) methoxy]pyrimidin-2-yl}morpholin-2-yl)methyl](methyl) amine (11.34 mg, 0.03 mmol, 17.13%) as a white glass (Purity: >95%).

### Example 230

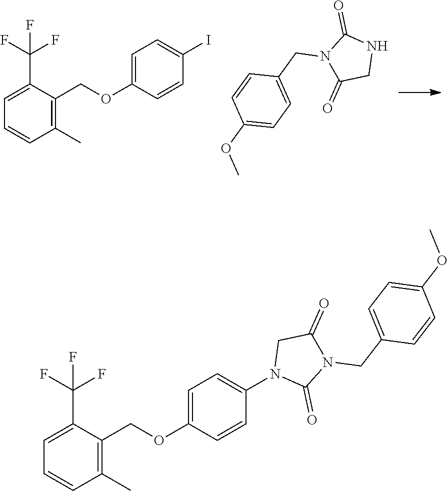 [SMILES: Cc1cccc(C(F)(F)F)c1COc1ccc(I)cc1 ; COc1ccc(CN2C(=O)CNC2=O)cc1 ; COc1ccc(CN2C(=O)CN(c3ccc(OCc4c(C)cccc4C(F)(F)F)cc3)C2=O)cc1]

2-(4-iodophenoxymethyl)-1-methyl-3-(trifluoromethyl)benzene (135 mg, 0.34 mmol, 1 eq), 1-[(4-methoxyphenyl)methyl]imidazolidine-2,4-dione, 3540-JSM-041-001 (113.72 mg, 0.52 mmol, 1.5 eq) and COPPER (I) OXIDE (49.26 mg, 0.34 mmol, 1 eq) were diluted with DMF (0.85 mL) under nitrogen and stirred at 150° C. for 2 Hours. To the reaction mixture was added 1-[(4-methoxyphenyl)methyl]imidazolidine-2,4-dione (22 mg, 0.10 mmol, 0.3 eq) and stirred under nitrogen at 150° C. for 4 Hours. The reaction mixture was filtered through celite, eluting with EtOAc, washed with a saturated aqueous solution of ammonium chloride (40 mL), 5% aqueous lithium chloride (30 mL), passed through hydrophobic filter paper and concentrated under reduced pressure to give a brown gum (217 mg). The crude mixture was purified by automated flash chromatography (Combiflash 300+, Silica 12 g RediSep column) eluting with 0 to 50% Ethyl Acetate in Heptaneate in Heptane Fractions (6-10) containing the desired product were concentrated under reduced pressure to give 1-[(4-methoxyphenyl)methyl]-3-(4-{[2-methyl-6-(trifluoromethyl)phenyl] methoxy}phenyl) imidazolidine-2,4-dione (102 mg, 0.21 mmol, 61.16%) as a peach solid (Purity: >98%)

### Example 231

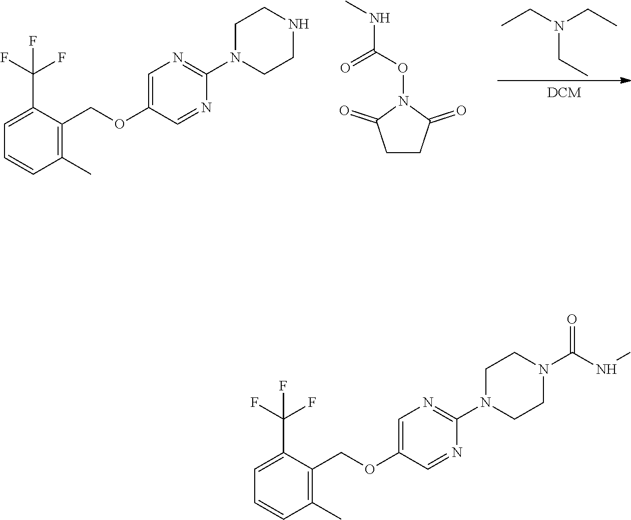

N-Succinimidyl N-methylcarbamate, 97% (42.8 mg, 0.25 mmol, 1.2 eq) was added to a stirred solution of 5-{[2-methyl-6-(trifluoromethyl)phenyl] methoxy}-2-(piperazin-1-yl)pyrimidine (73 mg, 0.21 mmol, 1 eq) and TRIETHYLAMINE (0.06 mL, 0.73 g/mL, 0.41 mmol, 2 eq) in DCM (3 mL). The reaction was stirred at rt for ca 3 h. The reaction was quenched with MeOH then the solvent removed in vacuo. The resultant residue was dissolved in DMSO, filtered and purified by preparative HPLC automated chromatography (ISCO ACCQ HP150, Prep HPLC Column: Gemini-NX C18 Dimensions: 21 mm×150 mm 5 μM; Sample: 1.50 ml from tube 1) eluting with 5 to 95% ACN/0.1% FA in Water/0.1% FA. Fractions 5 & 6 were combined then freeze dried. Gave N-methyl-4-(5-{[2-methyl-6-(trifluoromethyl)phenyl] methoxy}pyrimidin-2-yl) piperazine-1-carboxamide (20.66 mg, 0.05 mmol, 24.36%) as a white solid (Purity: >95%).

### Example 232

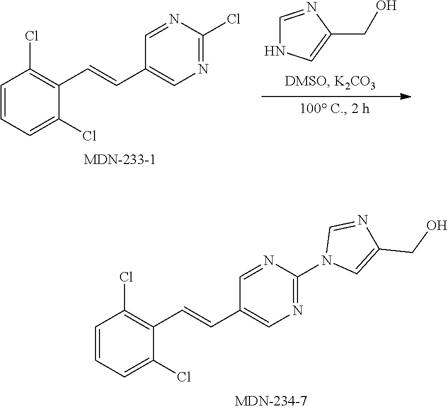 [SMILES: Clc1ncc(/C=C/c2c(Cl)cccc2Cl)cn1 ; OCc1c[nH]cn1 ; OCc1cn(-c2ncc(/C=C/c3c(Cl)cccc3Cl)cn2)cn1]

To a solution of MDN_233-1 (100 mg, 0.351 mmol) in DMSO (1 mL) were added (1H-imidazol-4-yl) methanol hydrochloride (107 mg, 0.669 mmol) and K₂CO₃ (145 mg, 1.05 mmol). The reaction was stirred at 100oC for 2 h. The reaction mixture was diluted with H₂O (30 mL) and extracted with EtOAc (30 mL×3). The organic layer was dried over Na2SO4, filtered and concentrated to give the crude product, which was purified by flash chromatography on silica gel (EA/PE=1:10) to afford the title compound (13 mg, 10.7% yield).

### Example 233

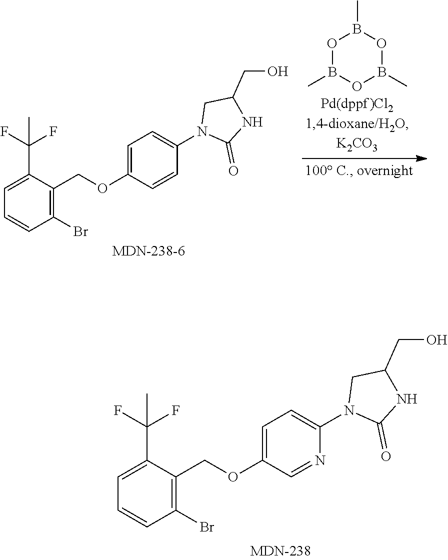

To a solution of MDN_238-6 (72 mg, 0.163 mmol) in 1,4-dioxane (1 ml) were added 2,4,6-trimethyl-1,3,5,2,4,6-trioxatriborinane (0.2 ml, 0.816 mmol, 3.5M in THF), Pd (dppf) C₁₂ (12 mg, 0.016 mmol), K₂CO₃ (317 mg, 2.30 mmol) and H2O (0.1 ml). The reaction was stirred at 100oC under Ar atmosphere overnight. The reaction mixture was diluted with H2O (20 ml) and extracted with EtOAc (20 ml×3). The organic layer was dried over Na2SO4, filtered and concentrated to give the crude product, which was purified by flash chromatography on silica gel (MeOH/DCM=0-10%) to afford the title compound as a white solid (44.4 mg, 60.3% yield).

### Example 234

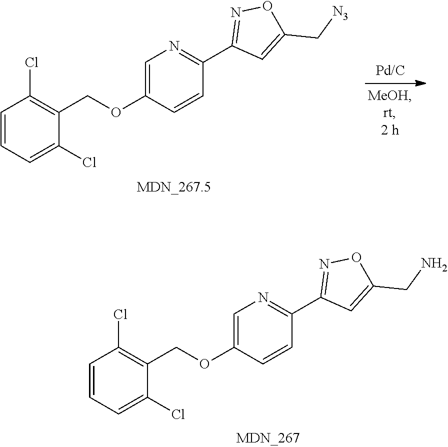 [SMILES: [N-]=[N+]=NCc1cc(-c2ccc(OCc3c(Cl)cccc3Cl)cn2)no1 ; NCc1cc(-c2ccc(OCc3c(Cl)cccc3Cl)cn2)no1]

To a solution of MDN_267-5 (250 mg, 0.660 mmol) in MeOH (5 mL) was added Pd/C (0.050 g). The reaction was stirred at rt for 2 h under H2 atmosphere. The mixture was filtered and the filtrate was purified by prep-HPLC (NH4HCO3) to obtain MDN_267 (20 mg, 8.7% yield, as a white solid).

### Example 235

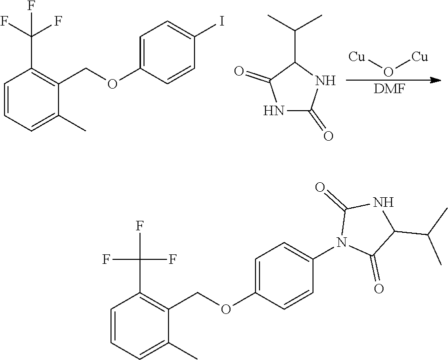

Nitrogen was bubbled through a DMF (0.5 mL) solution of 2-(4-iodophenoxymethyl)-1-methyl-3-(trifluoromethyl)benzene (30 mg, 0.08 mmol, 1 eq), 5-Isopropylimidazolidine-2,4-dione (16.31 mg, 0.11 mmol, 1.5 eq) and COPPER (I) OXIDE (10.95 mg, 0.08 mmol, 1 eq) in a reaction vial. The vial was capped then heated to 150° C., where it was maintained overnight. The reaction was cooled to rt then diluted with EtOAc and washed with sat. aq. NH4Cl followed by 5% w/v aq LiCl solution (x2). The organic extract was dried (MgSO4) then the solvent removed in vacuo. The resultant residue was dissolved in DMSO, filtered then purified by preparative HPLC automated chromatography (ISCO ACCQ HP150, Prep HPLC Column: Gemini-NX C18 Dimensions: 21 mm×150 mm 5 μM; Sample: 1.30 ml from tube 1) eluting with 5 to 95% ACN/0.1% FA in Water/0.1% FA Fraction 6 (3549-RJP-067-001) was freeze dried. Gave 5-isopropyl-3-(4-{[2-methyl-6-(trifluoromethyl)phenyl] methoxy}phenyl) imidazolidine-2,4-dione (4.32 mg, 0.01 mmol, 13.9%) as a white solid (Purity: >95%).

### Example 236

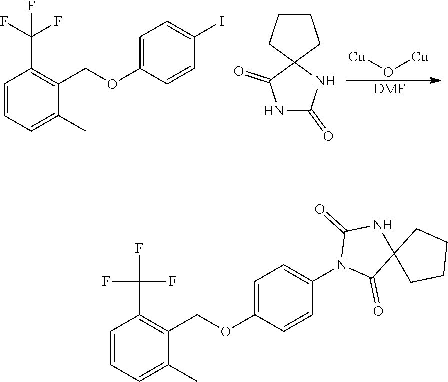

Nitrogen was bubbled through a DMF (0.5 mL) solution of 2-(4-iodophenoxymethyl)-1-methyl-3-(trifluoromethyl)benzene (30 mg, 0.08 mmol, 1 eq), 1,3-Diazaspiro[4.4]nonane-2,4-dione (17.69 mg, 0.11 mmol, 1.5 eq) and COPPER (I) OXIDE (10.95 mg, 0.08 mmol, 1 eq) in a reaction vial. The vial was capped then heated to 150° C., where it was maintained overnight. The reaction was cooled to rt then diluted with EtOAc and washed with sat. aq. NH₄Cl followed by 5% w/v aq LiCl solution (x2). The organic extract was dried (MgSO4) then the solvent removed in vacuo. The resultant residue was dissolved in DMSO, filtered then purified by preparative HPLC automated chromatography (ISCO ACCQ HP150, Prep HPLC Column: Gemini-NX C18 Dimensions: 21 mm×150 mm 5 μM; Sample: 1.30 ml from tube 1) eluting with 5 to 95% ACN/0.1% FA in Water/0.1% FA Fractions 3-5 were combined then freeze dried. Gave 3-(4-{[2-methyl-6-(trifluoromethyl)phenyl] methoxy}phenyl)-1,3-diazaspiro[4.4]nonane-2,4-dione (6.84 mg, 0.02 mmol, 21.37%) as a white solid (Purity: >95%).

### Example 237

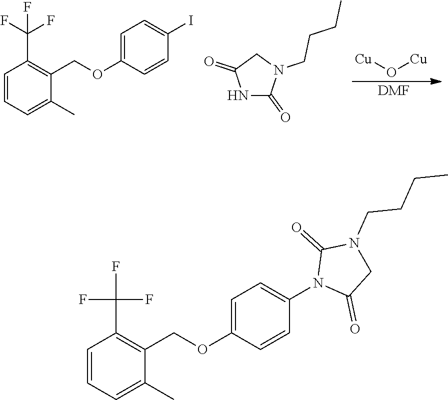

Nitrogen was bubbled through a DMF (0.5 mL) solution of 2-(4-iodophenoxymethyl)-1-methyl-3-(trifluoromethyl)benzene (30 mg, 0.08 mmol, 1 eq), 1-Butylhydantoin (17.92 mg, 0.11 mmol, 1.5 eq) and COPPER (I) OXIDE (10.95 mg, 0.08 mmol, 1 eq) in a reaction vial. The vial was capped then heated to 15 0oC, where it was maintained overnight. The reaction was cooled to rt then diluted with EtOAc and washed with sat. aq. NH₄Cl followed by 5% w/v aq LiCl solution (x2). The organic extract was dried (MgSO4) then the solvent removed in vacuo. The resultant residue was dissolved in DMSO, filtered then purified by preparative HPLC automated chromatography (ISCO ACCQ HP150, Sample: 1.30 ml from tube 1; Prep HPLC Column: Gemini-NX C18 Dimensions: 21 mm×150 mm 5 M) eluting with 5 to 95% ACN/0.1% FA in Water/0.1% FA. Fraction 8 was freeze dried. Gave 1-butyl-3-(4-{[2-methyl-6-(trifluoromethyl)phenyl] methoxy}phenyl) imidazolidine-2,4-dione (4 mg, 0.01 mmol, 12.44%) as a white solid (Purity: >95%).

### Example 238

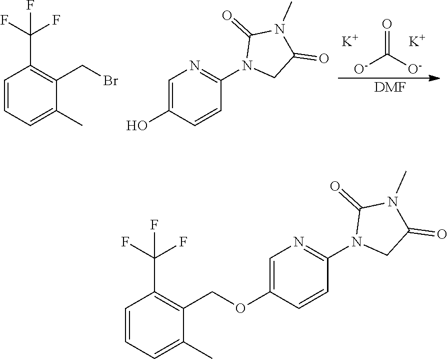

2-METHYL-6-TRIFLUOROMETHYLBENZYL BROMIDE (58.63 mg, 0.23 mmol, 1 eq), 1-(5-hydroxypyridin-2-yl)-3-methylimidazolidine-2,4-dione (48 mg, 0.23 mmol, 1 eq) and POTASSIUM CARBONATE (41.62 mg, 0.3 mmol, 1.3 eq) in DMF (2 mL) was stirred at rt for ca 4 h. The reaction was quenched with water then extracted with EtOAc (×2). The organic extract was washed with brine then dried (MgSO4) and the solvent removed in vacuo. The resultant residue was purified by preparative HPLC automated chromatography (ISCO ACCQ HP150, Prep HPLC Column: Gemini-NX C18 Dimensions: 21 mm×150 mm 5 μM; Sample: 1.30 ml from tube 1) eluting with 5 to 95% ACN/0.1% ammonia in Water/0.1% ammonia. Fraction 16 was freeze dried giving 3-methyl-1-(5-{[2-methyl-6-(trifluoromethyl)phenyl] methoxy}pyridin-2-yl) imidazolidine-2,4-dione (9.58 mg, 0.03 mmol, 10.9%) as a white solid (Purity: >95%).

### Example 239

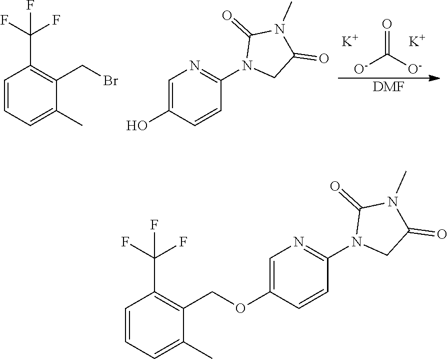

2-METHYL-6-TRIFLUOROMETHYLBENZYL BROMIDE (58.63 mg, 0.23 mmol, 1 eq), 1-(5-hydroxypyridin-2-yl)-3-methylimidazolidine-2,4-dione (48 mg, 0.23 mmol, 1 eq) and POTASSIUM CARBONATE (41.62 mg, 0.3 mmol, 1.3 eq) in DMF (2 mL) was stirred at rt for ca 4 h. The reaction was quenched with water then extracted with EtOAc (x2). The organic extract was washed with brine then dried (MgSO4) and the solvent removed in vacuo. The resultant residue was purified by preparative HPLC automated chromatography (ISCO ACCQ HP150, Prep HPLC Column: Gemini-NX C18 Dimensions: 21 mm×150 mm 5 μM; Sample: 1.30 ml from tube 1) eluting with 5 to 95% ACN/0.1% ammonia in Water/0.1% ammonia. Fraction 16 was freeze dried giving 3-methyl-1-(5-{[2-methyl-6-(trifluoromethyl)phenyl] methoxy}pyridin-2-yl) imidazolidine-2,4-dione (9.58 mg, 0.03 mmol, 10.9%) as a white solid (Purity: >95%).

### Example 240

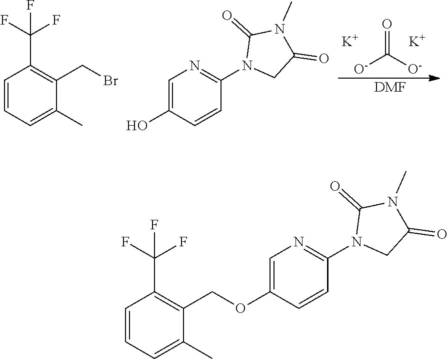

2-METHYL-6-TRIFLUOROMETHYLBENZYL BROMIDE (58.63 mg, 0.23 mmol, 1 eq), 1-(5-hydroxypyridin-2-yl)-3-methylimidazolidine-2,4-dione (48 mg, 0.23 mmol, 1 eq) and POTASSIUM CARBONATE (41.62 mg, 0.3 mmol, 1.3 eq) in DMF (2 mL) was stirred at rt for ca 4 h. The reaction was quenched with water then extracted with EtOAc (×2). The organic extract was washed with brine then dried (MgSO4) and the solvent removed in vacuo. The resultant residue was purified by preparative HPLC automated chromatography (ISCO ACCQ HP150, Prep HPLC Column: Gemini-NX C18 Dimensions: 21 mm×150 mm 5 μM; Sample: 1.30 ml from tube 1) eluting with 5 to 95% ACN/0.1% ammonia in Water/0.1% ammonia. Fraction 16 was freeze dried giving 3-methyl-1-(5-{[2-methyl-6-(trifluoromethyl)phenyl] methoxy}pyridin-2-yl) imidazolidine-2,4-dione (9.58 mg, 0.03 mmol, 10.9%) as a white solid (Purity: >95%).

### Example 241

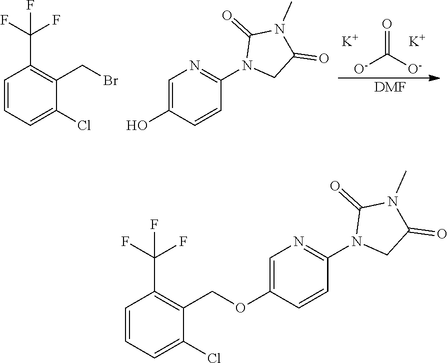

2-Chloro-6-(trifluoromethyl)benzyl bromide (0.04 mL, 1.66 g/cm³, 0.23 mmol, 1 eq), 1-(5-hydroxypyridin-2-yl)-3-methylimidazolidine-2,4-dione (48 mg, 0.23 mmol, 1 eq) and POTASSIUM CARBONATE (96.06 mg, 0.7 mmol, 3 eq) in DMF (2 mL) was stirred at rt for ca 2.5 h. The reaction was quenched with water then extracted with EtOAc (×2). The organic extract washed with brine, dried (MgSO4) and the solvent removed in vacuo. The resultant residue was dissolved in DMSO, filtered then purified by preparative HPLC automated chromatography (ISCO ACCQ HP150, Prep HPLC Column: Gemini-NX C18 Dimensions: 21 mm×150 mm 5 μM; Sample: 1.30 ml from tube 1) eluting with 5 to 95% ACN/0.1% FA in Water/0.1% FA Fraction 13 was freeze dried giving 1-(5-{[2-chloro-6-(trifluoromethyl)phenyl] methoxy}pyridin-2-yl)-3-methylimidazolidine-2,4-dione (22.99 mg, 0.06 mmol, 24.82%) as a white solid (Purity: >95%).

### Example 242

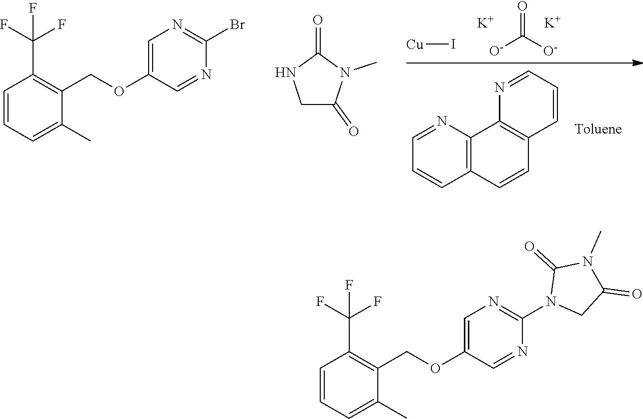

Nitrogen was bubbled through a Toluene (1 mL) solution of 2-bromo-5-{[2-methyl-6-(trifluoromethyl)phenyl] methoxy}pyrimidine (50 mg, 0.14 mmol, 1 eq), 3-methylimidazolidine-2,4-dione (16.44 mg, 0.14 mmol, 1 eq), 1,10-PHENANTHROLINE (2.6 mg, 0.01 mmol, 0.1 eq) and POTASSIUM CARBONATE (25.88 mg, 0.19 mmol, 1.3 eq) then COPPER (I) IODIDE (1.37 mg, 0.01 mmol, 0.05 eq) added. The pressure tube was sealed then heated to 140° C., where it was maintained overnight. LCMS shows the reaction has gone to completion. The reaction was cooled to rt then diluted with EtOAc. The organic extract was washed with water followed by brine then dried (MgSO4) and the solvent removed in vacuo. The resultant residue was dissolved in DMSO, filtered then purified by preparative HPLC automated chromatography (ISCO ACCQ HP150, Prep HPLC Column: Gemini-NX C18 Dimensions: 21 mm×150 mm 5 μM; Sample: 1.30 ml from tube 1) eluting with 5 to 95% ACN/0.1% FA in Water/0.1% FA. Fraction 9 was freeze dried giving 3-methyl-1-(5-{[2-methyl-6-(trifluoromethyl)phenyl] methoxy}pyrimidin-2-yl) imidazolidine-2,4-dione (10.21 mg 0.03 mmol, 18.64%) as a white solid (Purity: >95%).

### Example 243

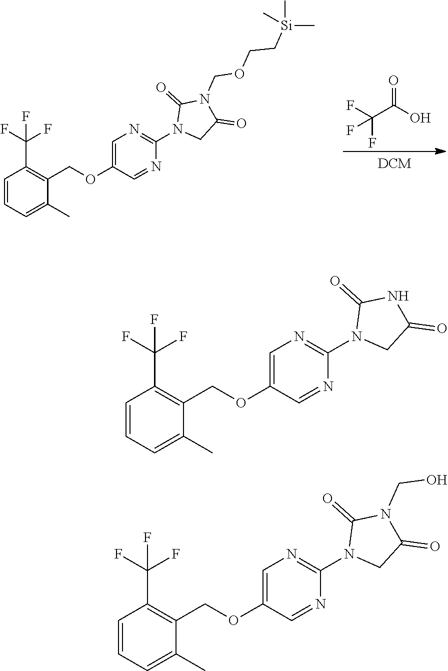 [SMILES: Cc1cccc(C(F)(F)F)c1COc1cnc(N2CC(=O)N(COCC[Si](C)(C)C)C2=O)nc1 ; O=C(O)C(F)(F)F ; Cc1cccc(C(F)(F)F)c1COc1cnc(N2CC(=O)NC2=O)nc1 ; Cc1cccc(C(F)(F)F)c1COc1cnc(N2CC(=O)N(CO)C2=O)nc1]

TRIFLUOROACETIC ACID (0.3 mL, 1.53 g/mL, 4.03 mmol, 18.01 eq) was added to a stirred solution of 1-(5-{[2-methyl-6-(trifluoromethyl)phenyl] methoxy}pyrimidin-2-yl)-3-{[2-(trimethylsilyl) ethoxy]methyl}imidazolidine-2,4-dione (111 mg, 0.22 mmol, 1 eq) in DCM (3 mL). The reaction was stirred at rt overnight. The solvent was removed in vacuo then the resultant residue dissolved in DMSO, filtered and purified by preparative HPLC automated chromatography (ISCO ACCQ HP150, Prep HPLC Column: Gemini-NX C18 Dimensions: 21 mm×150 mm 5 M; Sample: 1.30 ml from tube 1) eluting with 5 to 95% ACN/0.1% FA in Water/0.1% FA Fractions 5 & 6 were combined then freeze dried giving 3-(hydroxymethyl)-1-(5-{[2-methyl-6-(trifluoromethyl)phenyl] methoxy}pyrimidin-2-yl) imidazolidine-2,4-dione (46.63 mg, 0.12 mmol, 52.63%) as a white solid (Purity: >95%) (FORMATE, 0.15 eq).

### Example 244

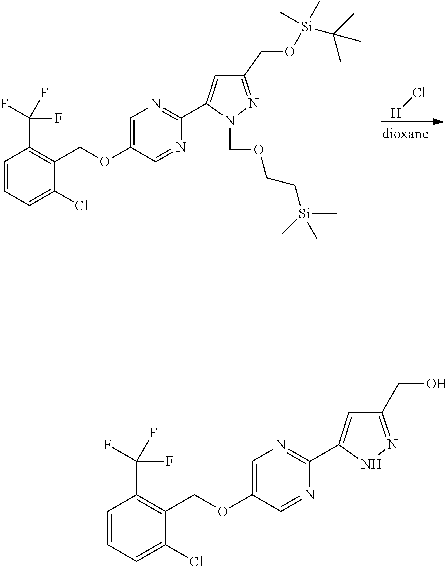 [SMILES: CC(C)(C)[Si](C)(C)OCc1cc(-c2ncc(OCc3c(Cl)cccc3C(F)(F)F)cn2)n(COCC[Si](C)(C)C)n1 ; Cl ; OCc1cc(-c2ncc(OCc3c(Cl)cccc3C(F)(F)F)cn2)[nH]n1]

2-(5-{[(tert-butyldimethylsilyl)oxy] methyl}-2-{[2-(trimethylsilyl) ethoxy]methyl}pyrazol-3-yl)-5-{[2-chloro-6-(trifluoromethyl)phenyl] methoxy}pyrimidine (65 mg, 0.1 mmol, 1 eq) was taken up in 1,4-Dioxane (0.5 mL) and hydrochloric acid (3.1 mL, 4M in, 12.4 mmol, 120 eq) (in dioxane) was added. Rxn was stirred at rt. After 1 h rxn complete by LC-MS. Rxn was conc in vacuo. Then taken up in 1 ml DMSO and filtered. Purified on Acuprep. Gem pH4 H2O to 5/95% H2O/MeCN F6 contains product. Frozen in CO₂/acetone bath, then attached to freeze drier over night. [5-(5-{[2-chloro-6-(trifluoromethyl)phenyl] methoxy}pyrimidin-2-yl)-1H-pyrazol-3-yl]methanol (13.37 mg, 0.03 mmol, 33.64%) isolated as a white solid. 2 . . .

### Example 247

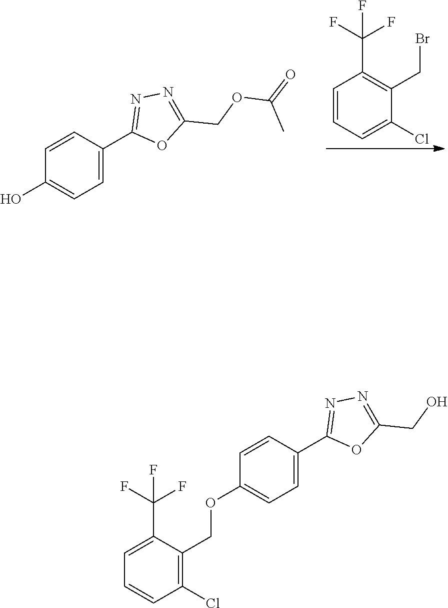

To a solution of the starting alcohol above (30 mg, 0.13 mmol, 1 eq) in DMF (2 mL) was added 2-Chloro-6-(trifluoromethyl)benzyl bromide (24.08 μL, 0.14 mmol, 1.1 eq) and potassium carbonate (35.41 mg, 0.26 mmol, 2 eq). Stirred at rt overnight. Water (200 μL) was added and stirred and stirred at rt for 2 hours. Heated on microwave at 80 C for 2 hours The reaction mixture was conc in vacuum, diluted with DMSO (2.6 mL), filtered and purified at pH4 on accq125 fractions 6-7 to give: [5-(4-{[2-chloro-6-(trifluoromethyl)phenyl] methoxy}phenyl)-1,3,4-oxadiazol-2-yl]methanol (29 mg, 0.08 mmol, 58.85%) white solid (Purity: >98%).

### Example 248

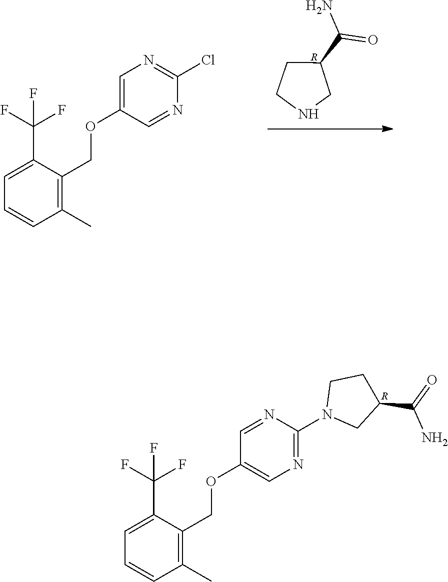

To a solution of the starting compound above (50 mg, 0.17 mmol, 1 eq) in ethanol (2 mL) was added (R)-Pyrrolidine-3-carboxamide (18.86 mg, 0.17 mmol, 1 eq) and Hunigs Base (57.55 μL, 0.33 mmol, 2 eq). Stirred on a seal tube at 120° C. for 16 hours. The reaction mixture was conc in vacuum, diluted with DMSO (2.6 mL), filtered and purified by preparative HPLC automated chromatography (ISCO ACCQ HP125, Sample: 2.00 ml from tube 1; Prep HPLC Column: Gemini-NX C18 Dimensions: 21 mm×150 mm 5 M) eluting with 10 to 95% ACN/0.1% FA in Water/0.1% FA fractions 20-21 to give: (3R)-1-(5-{[2-methyl-6-(trifluoromethyl)phenyl] methoxy}pyrimidin-2-yl) pyrrolidine-3-carboxamide (32 mg, 0.08 mmol, 50.93%) white solid (Purity: >98%).

### Example 249

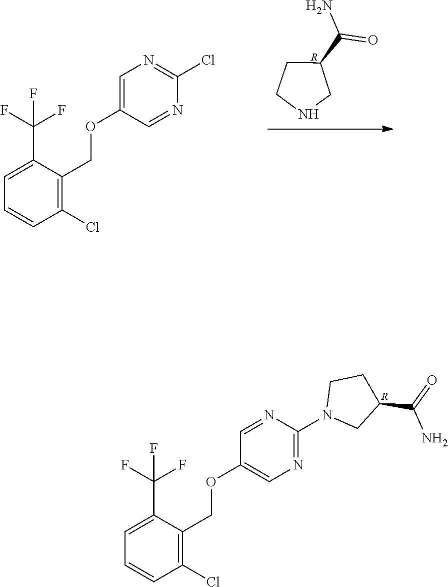

To a solution of the starting compound above (50 mg, 0.15 mmol, 1 eq) in ethanol (2 mL) was added (R)-Pyrrolidine-3-carboxamide (17.66 mg, 0.15 mmol, 1 eq) and Hunigs Base (53.91 μL, 0.31 mmol, 2 eq). Stirred on a seal tube at 120° C. for 16 hours. The reaction mixture was conc in vacuum, diluted with DMSO (2.6 mL), filtered and purified by preparative HPLC automated chromatography (ISCO ACCQ HP125, Sample: 2.60 ml from tube 2; Prep HPLC Column: Gemini-NX C18 Dimensions: 21 mm×150 mm 5 M) eluting with 10 to 95% ACN/0.1% FA in Water/0.1% FA fractions 30-31 to give: (3R)-1-(5-{[2-chloro-6-(trifluoromethyl)phenyl] methoxy}pyrimidin-2-yl) pyrrolidine-3-carboxamide (35 mg, 0.09 mmol, 56.43%) white solid (Purity: >98%).

### Example 253

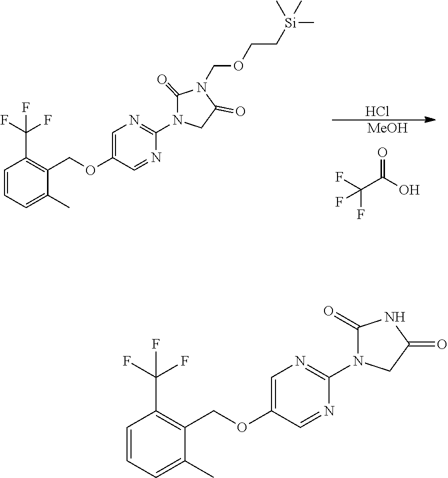 [SMILES: "MeOH" ; Cc1cccc(C(F)(F)F)c1COc1cnc(N2CC(=O)N(COCC[Si](C)(C)C)C2=O)nc1 ; O=C(O)C(F)(F)F ; Cl ; Cc1cccc(C(F)(F)F)c1COc1cnc(N2CC(=O)NC2=O)nc1]

HYDROCHLORIC ACID (3M in MeOH) (0.12 mL, 3M in MeOH, 0.36 mmol, 3 eq) was added to a stirred solution of 1-(5-{[2-methyl6-(trifluoromethyl)phenyl] methoxy}pyrimidin-2-yl)-3-{[2-(trimethylsilyl) ethoxy]methyl}imidazolidine-2,4-dione (59 mg, 0.12 mmol, 1 eq) in Methanol (3 mL). Ther eaction was stirred at rt overnight. The solvent was removed in vacuo then the resultant residue dissolved in DCM (3 mL) and TRIFLUOROACETIC ACID (0.03 mL, 1.53 g/mL, 0.36 mmol, 3 eq) added. The reaction was stirred at rt for ca 3 h. The solvent was removed in vacuo then the resultant residue dissolved in THF (3 mL) and 2N aq NaOH (1 mL) added. The reaction was stirred at rt for ca 20 min. The reaction was diluted with EtOAc then washed with water (small amount of brine added to aid separation). The aq was further extracted with EtOAc then the organic extracts combined and dried (MgSO4). The solvent was removed in vacuo from the recovered material (contaminated from the bottom of the fume hood). The aq was further extracted with DCM (x2) then the organic extract dried (MgSO4), combined with the recovered EtOAc extract and the solvent removed in vacuo. The resultant residue was dissolved in DMSO, filtered and purified by preparative HPLC automated chromatography (ISCO ACCQ HP150, Prep HPLC Column: Gemini-NX C18 Dimensions: 21 mm×150 mm 5 μM; Sample: 1.40 ml from tube 1) eluting with 5 to 95% ACN/0.1% FA in Water/0.1% FA. Fraction 3 was freeze dried. Gave 1-(5-{[2-methyl-6-(trifluoromethyl)phenyl] methoxy}pyrimidin-2-yl) imidazolidine-2,4-dione (8.91 mg, 0.02 mmol, 20.47%) as a white solid (Purity: >95%).

### Example 255

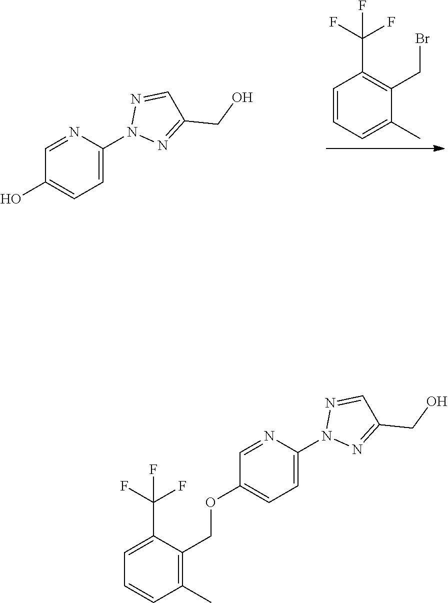 [SMILES: OCc1cnn(-c2ccc(O)cn2)n1 ; Cc1cccc(C(F)(F)F)c1CBr ; Cc1cccc(C(F)(F)F)c1COc1ccc(-n2ncc(CO)n2)nc1]

To a solution of the above starting material (30 mg, 0.16 mmol, 1 eq) in DMF (2 mL) was added 2-METHYL-6-TRIFLUOROMETHYLBENZYL BROMIDE (27.16 μL, 1.6 g/cm³, 0.17 mmol, 1.1 eq) and potassium carbonate (43.15 mg, 0.31 mmol, 2 eq). Stirred at rt over the weekend. The reaction mixture was filtered, washed with DMSO (1 mL) and purified by preparative HPLC automated chromatography (ISCO ACCQ HP125, Prep HPLC Column: Big Gemini-NX C18 Dimensions: 30 mm×250 mm 5 μM; Sample: 2.50 ml from tube 1) eluting with 5 to 95% ACN/0.1% FA in Water/0.1% FA fractions 15-17 to give: [2-(5-{[2-methyl-6-(trifluoromethyl)phenyl] methoxy}pyridin-2-yl)-1,2,3-triazol-4-yl]methanol (23 mg, 0.06 mmol, 40.44%) white solid (Purity: >95%).

### Example 256

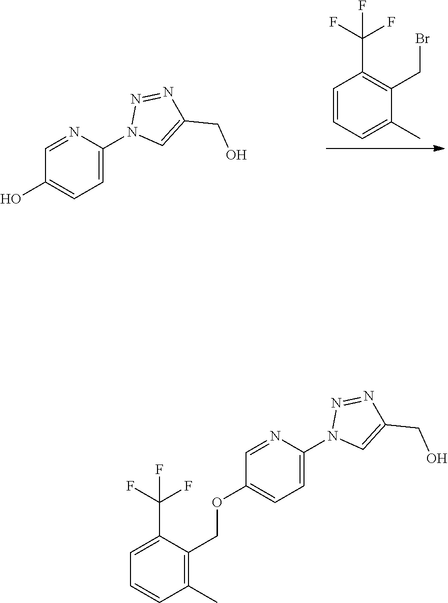 [SMILES: OCc1cn(-c2ccc(O)cn2)nn1 ; Cc1cccc(C(F)(F)F)c1CBr ; Cc1cccc(C(F)(F)F)c1COc1ccc(-n2cc(CO)nn2)nc1]

To a solution of the above starting material (14 mg, 0.07 mmol, 1 eq) in DMF (2 mL) was added 2-METHYL-6-TRIFLUOROMETHYLBENZYL BROMIDE (12.67 μL, 1.6 g/cm³, 0.08 mmol, 1.1 eq) and potassium carbonate (20.14 mg, 0.15 mmol, 2 eq). Stirred at rt over the week-end. The reaction mixture was filtered, washed with DMSO (1 mL) and purified by preparative HPLC automated chromatography (ISCO ACCQ HP125, Prep HPLC Column: Big Gemini-NX C18 Dimensions: 30 mm×250 mm 5 μM; Sample: 2.50 ml from tube 2) eluting with 5 to 95% ACN/0.1% FA in Water/0.1% FA fractions 50-52 to give: [1-(5-{[2-methyl-6-(trifluoromethyl)phenyl] methoxy}pyridin-2-yl)-1,2,3-triazol-4-yl]methanol (14 mg, 0.04 mmol, 52.75%) white solid (Purity: 85-90%).

### Example 257

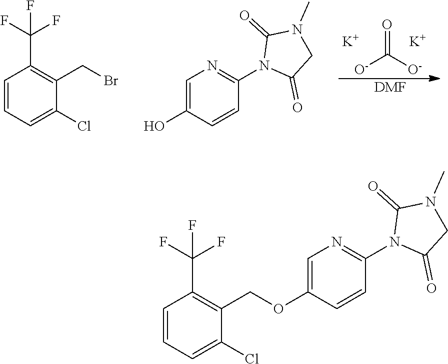

A solution of 2-Chloro-6-(trifluoromethyl)benzyl bromide (0.06 mL, 1.66 g/cm³, 0.36 mmol, 1 eq), 3-(5-hydroxypyridin-2-yl)-1-methylimidazolidine-2,4-dione (124 mg (60%), 0.36 mmol, 1 eq) and POTASSIUM CARBONATE (64.52 mg, 0.47 mmol, 1.3 eq) in DMF (3 mL) was stirred at rt overnight. LCMS shows product present plus several impurities. The reaction was diluted with EtOAc then washed with water followed by brine. The organic extract was dried (MgSO4) then the solvent removed in vacuo. The resultant residue was dissolved in DMSO, filtered then purified by preparative HPLC automated chromatography (ISCO ACCQ HP150, Prep HPLC Column: Gemini-NX C18 Dimensions: 21 mm×150 mm 5 μM; Sample: 1.40 ml from tube 1) eluting with 5 to 95% ACN/0.1% FA in Water/0.1% FA Fraction 14 was freeze dried giving 3-(5-{[2-chloro-6-(trifluoromethyl)phenyl] methoxy}pyridin-2-yl)-1-methylimidazolidine-2,4-dione (51.01 mg, 0.13 mmol, 35.53%) as a white solid (Purity: >95%).

### Example 258

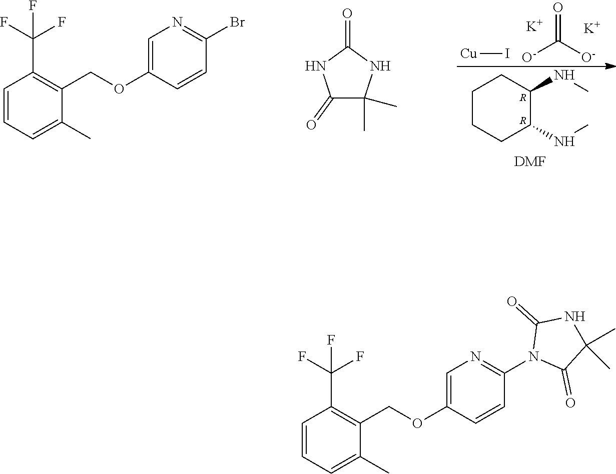

Nitrogen was bubbled through a DMF (4 mL) solution of 2-bromo-5-{[2-methyl-6-(trifluoromethyl)phenyl] methoxy}pyridine (90 mg, 0.26 mmol, 1 eq), 5,5-Dimethylhydantoin (33.31 mg, 0.26 mmol, 1 eq), COPPER (I) IODIDE (49.52 mg, 0.26 mmol, 1 eq) and POTASSIUM CARBONATE (71.87 mg, 0.52 mmol, 2 eq) in a pressure vial then TRANS-(1R,2R)-N,N′-BISMETHYL-1,2-CYCLOHEXANEDIAMINE (0.02 mL, 0.9 g/cm³, 0.1 mmol, 0.4 eq) added. The reaction was sealed then heated to 90oC, where it was maintained overnight. LCMS shows the reaction has gone to completion. The reaction was cooled to rt then diluted with EtOAc and washed with sat. aq. NaHCO3 solution followed by brine. The organic extract was dried (MgSO4) then the solvent removed in vacuo. The resultant residue was dissolved in DMSO, filtered then purified by preparative HPLC automated chromatography (ISCO ACCQ HP150, Prep HPLC Column: Gemini-NX C18 Dimensions: 21 mm×150 mm 5 μM; Sample: 1.30 ml from tube 1) eluting with 5 to 95% ACN/0.1% FA in Water/0.1% FA. Fraction 17 was freeze dried giving 5,5-dimethyl-3-(5-{[2-methyl-6-(trifluoromethyl)phenyl] methoxy}pyridin-2-yl) imidazolidine-2,4-dione (49.09 mg, 0.12 mmol, 48%) as a white solid (Purity: >95%).

### Example 259

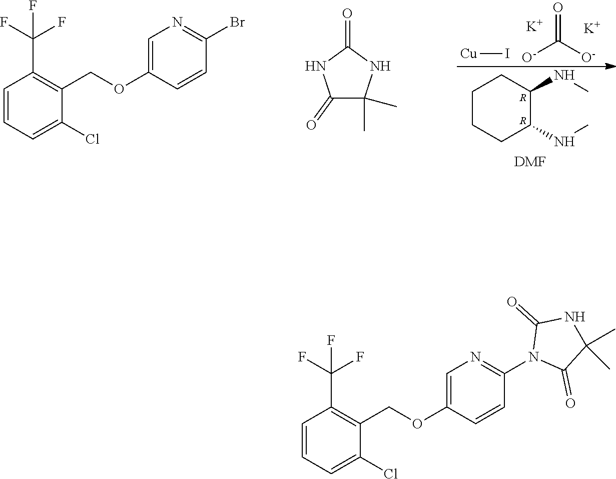

Nitrogen was bubbled through a DMF (4 mL) solution of 2-bromo-5-{[2-chloro-6-(trifluoromethyl)phenyl] methoxy}pyridine (100 mg, 0.27 mmol, 1 eq), 5,5-Dimethylhydantoin (34.95 mg, 0.27 mmol, 1 eq), POTASSIUM CARBONATE (75.41 mg, 0.55 mmol, 2 eq) and COPPER (I) IODIDE 51.96 mg, 0.27 mmol, 1 eq) in a pressure tube. trans-N,N′-Dimethylcyclohexane-1,2-diamine (0.02 mL, 0.9 g/cm³, 0.11 mmol, 0.4 eq) was added then the tube sealed and heated to 90oC, where it was maintained overnight. LCMS shows the reaction has gone to completion. The reaction was cooled to rt then diluted with EtOAc and washed with sat. aq. NaHCO3 solution followed by brine. The organic extract was dried (MgSO4) then the solvent removed in vacuo. The resultant residue was dissolved in DMSO, filtered then purified by preparative HPLC automated chromatography (ISCO ACCQ HP150, Sample: 1.40 ml from tube 1; Prep HPLC Column: Gemini-NX C18 Dimensions: 21 mm×150 mm 5 μM) eluting with 5 to 95% ACN/0.1% FA in Water/0.1% FA. Fractions 15-17 were combined then freeze dried giving 3-(5-{[2-chloro-6-(trifluoromethyl)phenyl] methoxy}pyridin-2-yl)-5,5-dimethylimidazolidine-2,4-dione (56.96 mg, 0.14 mmol, 50.46%) as a white solid (Purity: >95%).

### Example 260

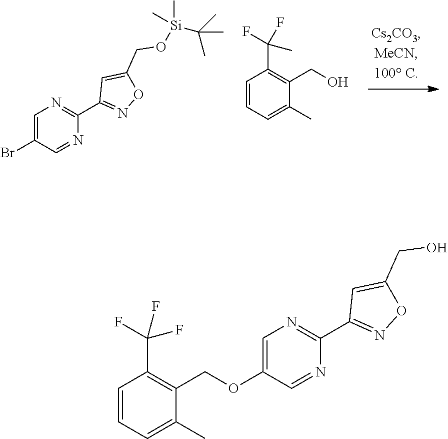

5-bromo-2-(5-{[(tert-butyldimethylsilyl)oxy] methyl}-1,2-oxazol-3-yl) pyrimidine (28 mg, 0.08 mmol, 1 eq) was taken up in Acetonitrile (1.5 mL) [2-methyl-6-(trifluoromethyl)phenyl] methanol (43.14 mg, 0.23 mmol, 3 eq) and cesium carbonate (49.27 mg, 0.15 mmol, 2 eq) were added. Heated in a sealed tube at 100oC behind a blast shield. Rxn was partitioned between ⅓ IPA/DCM and H2O. The organics were separated via a phase separator and conc in vacuo. The residue was taken up in 1 ml DMSO and filtered. purified by preparative HPLC automated chromatography (ISCO ACCQ HP150, Prep HPLC Column: Gemini-NX C18 Dimensions: 21 mm×150 mm 5 μM; Sample: 1.30 ml from tube 1) eluting with 5 to 95% ACN/0.1% FA in Water/0.1% FA F11 and F12 combined and frozen in a CO₂/acetone bath, then attached to the freeze drier. After 18 h, the residue was taken up in DCM and blown down into a submission vial. This was dried in vacuo at 40oC OVERNIGHT. [3-(5-{[2-methyl-6-(trifluoromethyl)phenyl] methoxy}pyrimidin-2-yl)-1,2-oxazol-5-yl] methanol (1.44 mg, 3.94 μmol, 5.21%) isolated as a white solid.

### Example 261

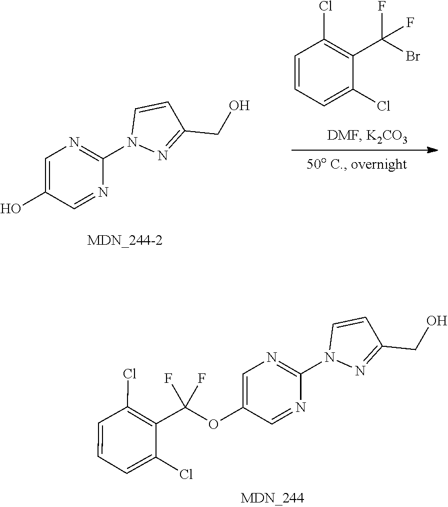 [SMILES: OCc1ccn(-c2ncc(O)cn2)n1 ; FC(F)(Br)c1c(Cl)cccc1Cl ; OCc1ccn(-c2ncc(OC(F)(F)c3c(Cl)cccc3Cl)cn2)n1]

To a solution of MDN_244-2 (30 mg, 0.165 mmol) in DMF (1 mL) was added MDN_244-1A (45 mg, 0.165 mmol) and K₂CO₃ (29 mg, 0.330 mmol). The reaction was stirred at 50oC overnight. The reaction mixture was diluted with H2O (10 mL) and extracted with EtOAc (10 mL×3). The organic layer was washed with brine (20 mL), dried over Na2SO4, filtered and concentrated in vacuo. The residue was purified by TLC (PE:EA=1:1) to afford MDN_244 as a white solid (11 mg, 16.9% yield).

### Example 262

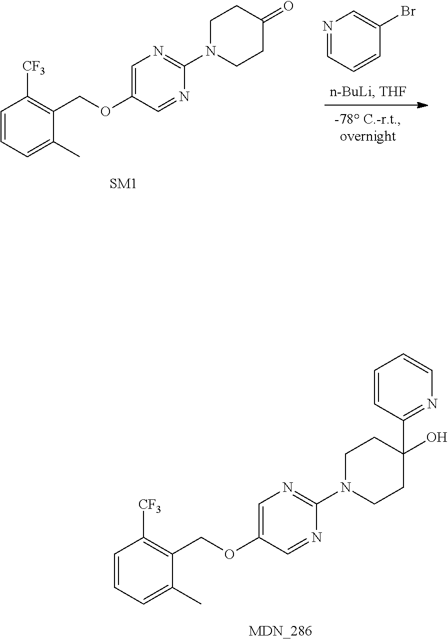

To a solution of 2-bromopyridine (66 mg, 0.422 mmol) in anhydrous THF (2 mL) was added n-BuLi (0.6 mL, 1.06 mmol) at −78oC dropwise. After stirring at this temperature for 0.5 h, 1-(5-((2-methyl-6-(trifluoromethyl)benzyl)oxy) pyrimidin-2-yl) piperidin-4-one (100 mg, 0.274 mmol) in THF (1 mL) was slowly added. The reaction was stirred at −78oC overnight. The reaction mixture was quenched with NH₄Cl (30 mL) and extracted with EtOAc (30 mL×3). The organic layer was dried over Na2SO4, filtered and concentrated in vacuo. The residue was purified by TLC (PE:EA=5:1) to afford MDN_286 as a white solid (30.0 mg, 31.9% yield).

### Example 263

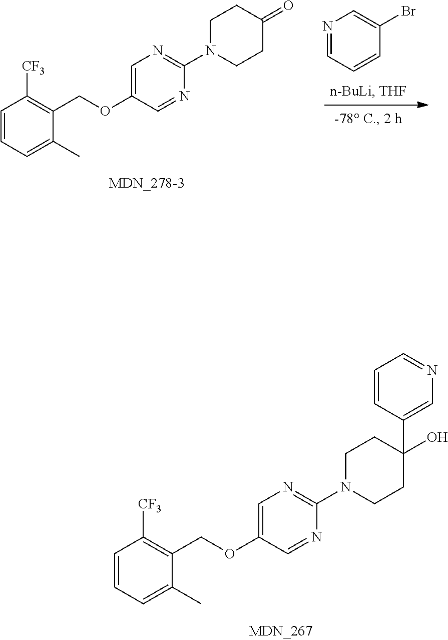

To a solution of 3-bromopyridine (33 mg, 0.209 mmol) in anhydrous THF (2 mL) was added n-BuLi (0.3 mL, 0.522 mmol) at −78oC dropwise. After stirring at this temperature for 0.5 h, MDN_278-3 (100 mg, 0.272 mmol) in THF (1 mL) was slowly added. The reaction was stirred at −78oC for 2 h. The reaction mixture was quenched with NH₄Cl (30 mL) and extracted with EtOAc (30 mL×3). The organic layer was dried over Na2SO4, filtered and concentrated in vacuo. The residue was purified by TLC (MeOH:DCM=0%˜10%) to afford MDN_287 as a white solid (22.0 mg, 27.9% yield).

### Example 264

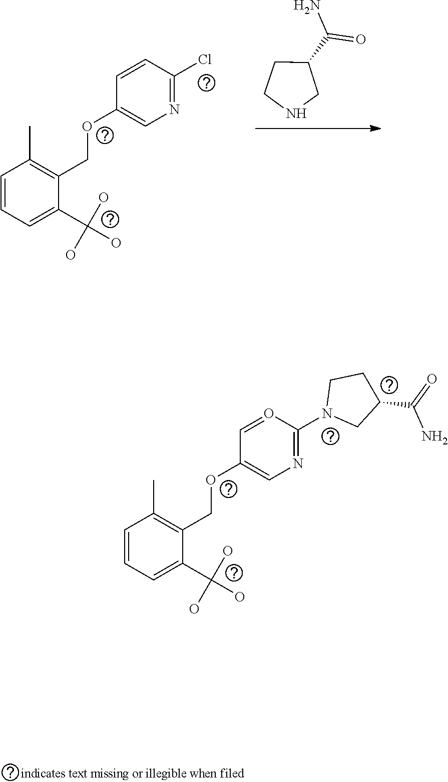

To a solution of the above starting compound (100 mg, 0.33 mmol, 1 eq) in ethanol (4 mL) was added(S)-Pyrrolidine-3-carboxamide (37.71 mg, 0.33 mmol, 1 eq) and Hunigs Base (0.12 mL, 0.66 mmol, 2 eq). Stirred on a seal tube at 120° C. for 16 hours. The reaction mixture was concentrated in vacuo, diluted with DMSO (2.6 mL), filtered and purified by preparative HPLC automated chromatography (ISCO ACCQ HP125, Sample: 2.80 ml from tube 1; Prep HPLC Column: Big Gemini-NX C18 Dimensions: 30 mm×250 mm 5 μM) eluting with 20 to 95% ACN/0.1% FA in Water/0.1% FA. Fractions 10-12 gave (3S)-1-(5-{[2-methyl-6-(trifluoromethyl)phenyl] methoxy}pyrimidin-2-yl) pyrrolidine-3-carboxamide (44 mg, 0.12 mmol, 35.01%) as a white solid (Purity: >95%).

### Example 265

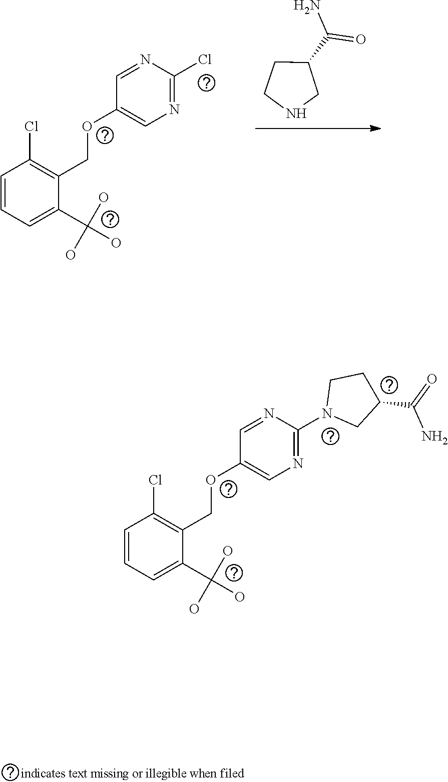 [SMILES: OC(O)(O)c1cccc(Cl)c1COc1cnc(Cl)nc1 ; NC(=O)[C@H]1CCNC1 ; NC(=O)[C@H]1CCN(c2ncc(OCc3c(Cl)cccc3C(O)(O)O)cn2)C1]

To a solution of the above starting compound (100 mg, 0.31 mmol, 1 eq) in ethanol (4 mL) was added(S)-Pyrrolidine-3-carboxamide (35.33 mg, 0.31 mmol, 1 eq) and Hunigs Base (0.11 mL, 0.62 mmol, 2 eq). Stirred in a seal tube at 120° C. for 16 hours. The reaction mixture was conc in vacuum, diluted with DMSO (2.6 mL), filtered and purified by preparative HPLC automated chromatography (ISCO ACCQ HP125, Sample: 2.70 ml from tube 2; Prep HPLC Column: Big Gemini-NX C18 Dimensions: 30 mm×250 mm 5 μM) eluting with 20 to 95% ACN/0.1% FA in Water/0.1% FA. Fractions 37-39 gave (3S)-1-(5-{[2-chloro-6-(trifluoromethyl)phenyl] methoxy}pyrimidin-2-yl) pyrrolidine-3-carboxamide (30 mg, 0.07 mmol, 24.18%) as a white solid (Purity: >95%).

### Example 266

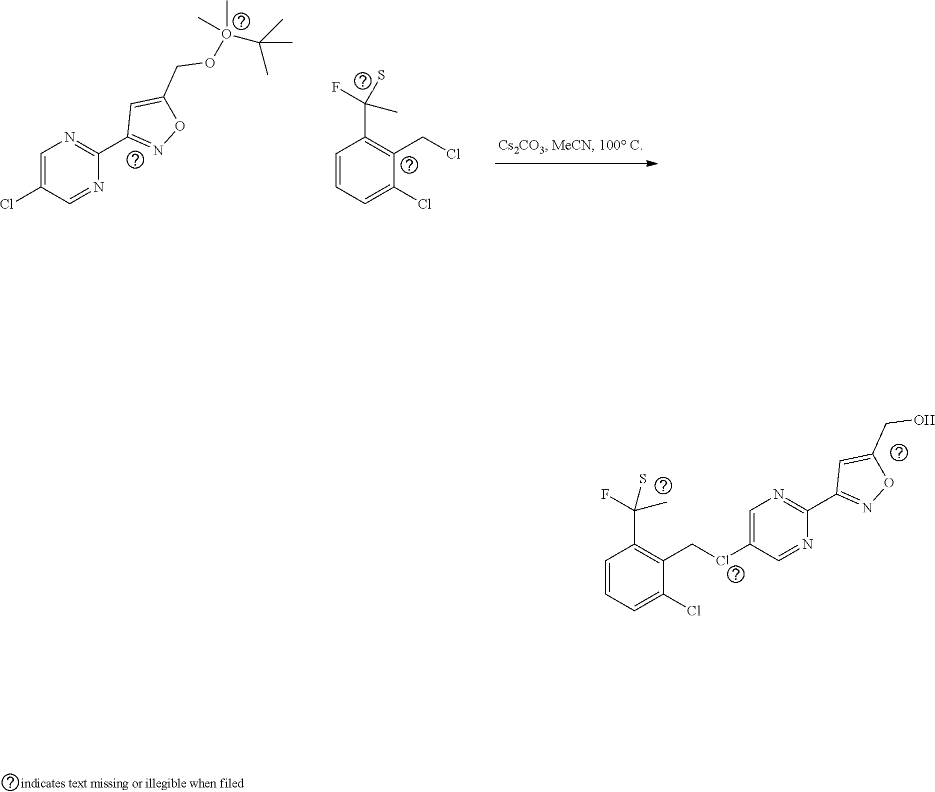

5-bromo-2-(5-{[(tert-butyldimethylsilyl)oxy] methyl}-1,2-oxazol-3-yl) pyrimidine (206 mg, 0.56 mmol, 1 eq) was taken up in Acetonitrile (10 mL) in a sealed tube. 2-Chloro-6-(trifluoromethyl)benzyl alcohol (351.43 mg, 1.67 mmol, 3 eq) was added followed by cesium carbonate (362.5 mg, 1.11 mmol, 2 eq). Rxn was sparged with N₂ then sealed and heated to 100° C. for 24 h. Rxn was partitioned between EtOAc x2/H2O. Combined organics were dried over MgSO4, filtered and conc in vacuo. The residue was taken up in 2 ml DMSO and filtered. purified by preparative HPLC automated chromatography (ISCO ACCQ HP150, Prep HPLC Column: Big Gemini-NX C18 Dimensions: 30 mm×250 mm 5 μM; Sample: 2.50 ml from tube 1) eluting with 5 to 95% ACN/0.1% FA in Water/0.1% FA purified by preparative HPLC automated chromatography (ISCO ACCQ HP150, Prep HPLC Column: Big Gemini-NX C18 Dimensions: 30 mm×250 mm 5 μM; Sample: 2.50 ml from tube 1) eluting with 5 to 95% ACN/0.1% FA in Water/0.1% FA. F43 and 44 combined and conc in vacuo. The residue was taken up in minimal DCM, then blown down into a submission vial and dried at 40° C. in vacuo OVERNIGHT to give [3-(5-{[2-chloro-6-(trifluoromethyl)phenyl] methoxy}pyrimidin-2-yl)-1,2-oxazol-5-yl]methanol (10.39 mg, 0.03 mmol, 4.84%) as a beige solid.

### Example 267

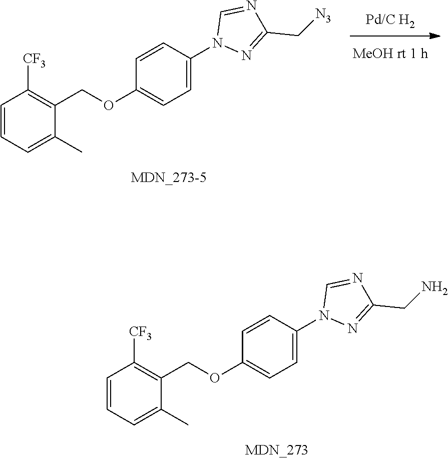 [SMILES: Cc1cccc(C(F)(F)F)c1COc1ccc(-n2cnc(CN=[N+]=[N-])n2)cc1 ; Cc1cccc(C(F)(F)F)c1COc1ccc(-n2cnc(CN)n2)cc1]

A mixture of MDN_273-5 (50 mg, 0.128 mmol) and Pd/C (10%, 10 mg) in MeOH (1 mL) was stirred at rt for 1 h under H2 balloon. The mixture was filtered, concentrated, purified by silica gel column (H2O: MeCN) to give the title compound MDN_273 as a white solid (20 mg, 43% yield).

### Example 268

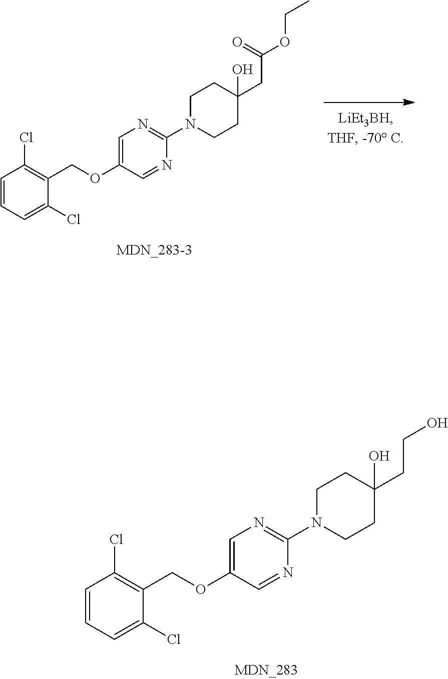 [SMILES: CCOC(=O)CC1(O)CCN(c2ncc(OCc3c(Cl)cccc3Cl)cn2)CC1 ; OCCC1(O)CCN(c2ncc(OCc3c(Cl)cccc3Cl)cn2)CC1]

To a solution of MDN_283-3 (50 mg, 0.12 mmol) in THF (1 mL) was added LiEt3BH (1M in THF, 0.57 mL, 0.57 mmol) dropwised at −70oC. The mixture was stirred at −70oC for 3 h. The mixture was quenched with water, extracted with EA and concentrated to give the residue, which was purified by chromatography on silica gel (EA/PE=0-20%) to give the title compound MDN_283 as a white solid (14 mg, 30% yield).

### Example 269

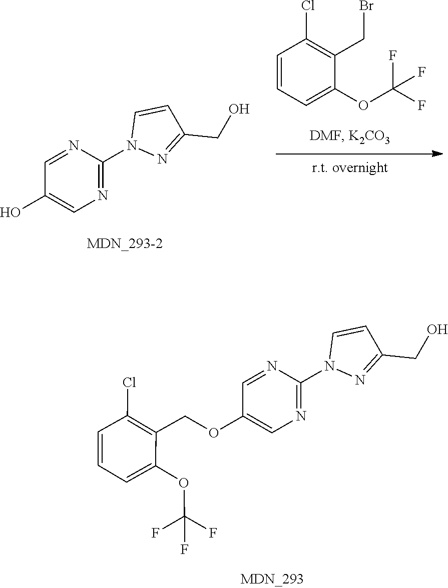 [SMILES: FC(F)(F)Oc1cccc(Cl)c1CBr ; OCc1ccn(-c2ncc(O)cn2)n1 ; OCc1ccn(-c2ncc(OCc3c(Cl)cccc3OC(F)(F)F)cn2)n1]

A mixture of MDN_293-2 (80 mg, 0.278 mmol), K₂CO₃ (77 mg, 0.556 mmol) and 2-(bromomethyl)-1-chloro-3-(trifluoromethoxy)benzene (54 mg, 0.278 mmol) in DMF (1 mL) was stirred at r.t. overnight. The reaction mixture was diluted with H2O (100 mL) and extracted with EtOAc (80 mL×3). The organic phase dried over Na2SO4, concentrated and the crude product was purified by silica gel column (EA/PE=0%˜50%) to give the title compound as a yellow solid (12 mg, 11.0% yield).

### Example 270

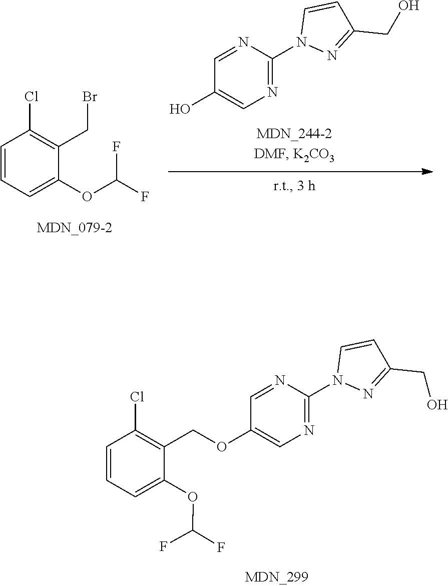 [SMILES: FC(F)Oc1cccc(Cl)c1CBr ; OCc1ccn(-c2ncc(O)cn2)n1 ; OCc1ccn(-c2ncc(OCc3c(Cl)cccc3OC(F)F)cn2)n1]

To a solution of MDN_079-2 (50 mg, 0.260 mmol) in DMF (1 mL) were added K2CO3 (72 mg, 0.520 mmol) and MDN_244-2 (71 mg, 0.260 mmol). The reaction was stirred at r.t. for 3 h. The reaction mixture was diluted with H2O (30 mL) and extracted with EtOAc (30 mL×3). The organic layer was dried over Na2SO4, filtered and concentrated in vacuo. The crude product was purified by tablet chromatography (PE:EA=1:1) to afford the title compound (23.6 mg, 23.7% yield, as a white solid).

### Example 271

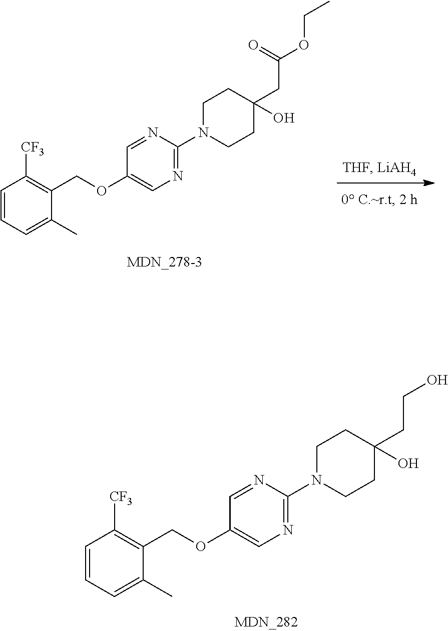 [SMILES: CCOC(=O)CC1(O)CCN(c2ncc(OCc3c(C)cccc3C(F)(F)F)cn2)CC1 ; Cc1cccc(C(F)(F)F)c1COc1cnc(N2CCC(O)(CCO)CC2)nc1]

To a solution of MDN_278-3 (100 mg, 0.230 mmol) in anhydrous THF (2 mL) was added LiAlH₄ (51.9 mg, 1.37 mmol) slowly at 0oC. The reaction was allowed to warm to room temperature and stirred for 2 h. The reaction mixture was quenched with H2O slowly at 0oC. The mixture was filtered through a pad of Celite and rinsed with EtOAc. The filtrate was extracted with EA (50 mL×3). The organic layer was dried over Na2SO4, filtered and concentrated in vacuo. The crude product was purified by TLC (PE:EA=1:1) to afford the title compound (3.3 mg, 3.5% yield) as a white solid.

### Example 272

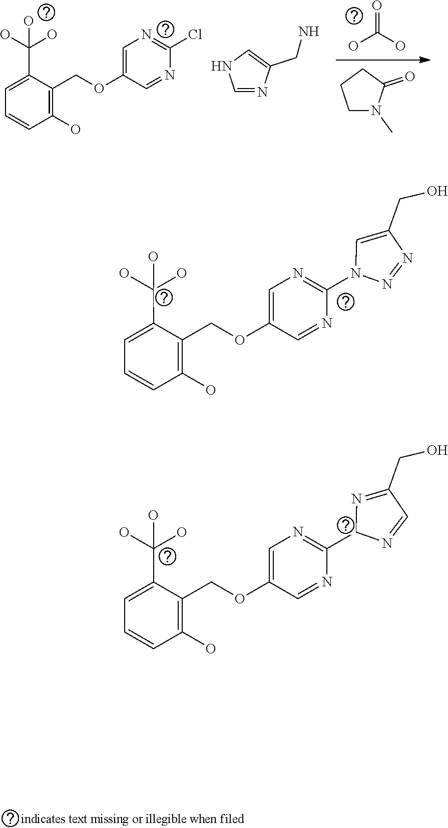

Nitrogen was bubbled through a solution of 2-chloro-5-{[2-chloro-6-(trifluoromethyl)phenyl] methoxy}pyrimidine (400 mg, 1.24 mmol, 1 eq), 1H-1,2,3-triazol-4-ylmethanol (122.68 mg, 1.24 mmol, 1 eq) and POTASSIUM CARBONATE (342.2 mg, 2.48 mmol, 2 eq) in 1-Methyl-2-pyrrolidinone (8 mL, 1.02 g/mL, 82.32 mmol, 66.49 eq). The pressure tube was sealed then heated to 80oC, where it was maintained overnight. The reaction was cooled to rt then diluted with EtOAc and washed with water followed by brine. The organic extract was dried (MgSO4) then the solvent removed in vacuo. The resultant residue was dissolved in DMSO then purified by preparative HPLC automated chromatography (ISCO ACCQ HP150, Prep HPLC Column: Big Gemini-NX C18 Dimensions: 30 mm×250 mm 5 μM; Sample: 1.20 ml from tube 2) eluting with 5 to 95% ACN/0.1% FA in Water/0.1% FA (2.4 ml total split over 2 injections). The desired fractions were combined then freeze dried giving [2-(5-{[2-chloro-6-(trifluoromethyl)phenyl] methoxy}pyrimidin-2-yl)-1,2,3-triazol-4-yl] methanol (36.49 mg, 0.09 mmol, 7.64%) as a white solid (Purity: >95%) and [1-(5-{[2-chloro-6-(trifluoromethyl)phenyl] methoxy}pyrimidin-2-yl)-1,2,3-triazol-4-yl]methanol (59.9 mg, 0.16 mmol, 12.54%) as a white solid (Purity: >98%) (ACETONITRILE, 0.11 eq).

### Example 273

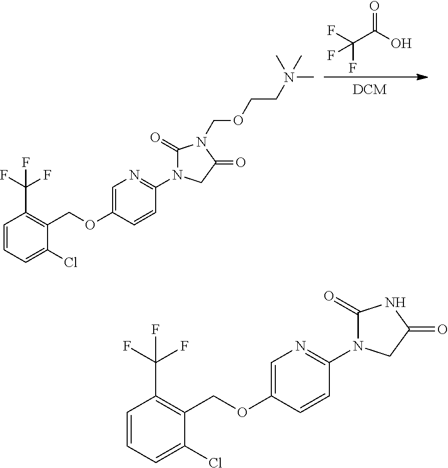

Trifluoroacetic acid, 99% (0.08 mL, 1.53 g/mL, 1.1 mmol, 4 eq) was added to a stirred solution of 1-(5-{[2-chloro-6-(trifluoromethyl)phenyl] methoxy}pyridin-2-yl)-3-{[2-(trimethylsilyl) ethoxy]methyl}imidazolidine-2,4-dione (142 mg, 0.28 mmol, 1 eq) in DCM (5 mL). The reaction was stirred at rt overnight. LCMS shows mainly product with MeOH linker still attached plus some starting material present. Further Trifluoroacetic acid, 99% (0.04 mL, 1.53 g/mL, 0.55 mmol, 2 eq) was added then stirring continued at rt for a further ca 6 h. The reaction was then stood at rt overnight. The solvent was removed in vacuo then the resultant residue dissolved in THF (2 mL) and 2N NaOH (aq) (1 mL) added. The reaction was stirred at rt for ca 1.5 h. The reaction was diluted with EtOAc then washed with water followed by brine. The organic extract was dried (MgSO4) then the solvent removed in vacuo. The resultant residue was dissolved in DMSO then purified by preparative HPLC automated chromatography (ISCO ACCQ HP150, Prep HPLC Column: Gemini-NX C18 Dimensions: 21 mm×150 mm 5 M; Sample: 1.60 ml from tube 1) eluting with 5 to 95% ACN/0.1% FA in Water/0.1% FA. Fractions 8-10 were combined then freeze dried giving 1-(5-{[2-chloro-6-(trifluoromethyl)phenyl] methoxy}pyridin-2-yl) imidazolidine-2,4-dione (56.46 mg, 0.15 mmol, 53.19%) as a solid (Purity: 85-90%). The sample was re-dissolved in THF (1 mL) then 2N aq NaOH added. The reaction was stirred at rt for ca 2.5 h. The reaction was diluted with EtOAc then washed with water followed by brine. The organic extract was dried (MgSO4) then the solvent removed in vacuo. The resultant solid was triturated in diethyl ether then collected by filtration and dried on under vacuum at 40° C. Gave 1-(5-{[2-chloro-6-(trifluoromethyl)phenyl] methoxy}pyridin-2-yl) imidazolidine-2,4-dione (41.33 mg, 0.11 mmol, 38.93%) as a white solid (Purity: >95%).

### Example 277

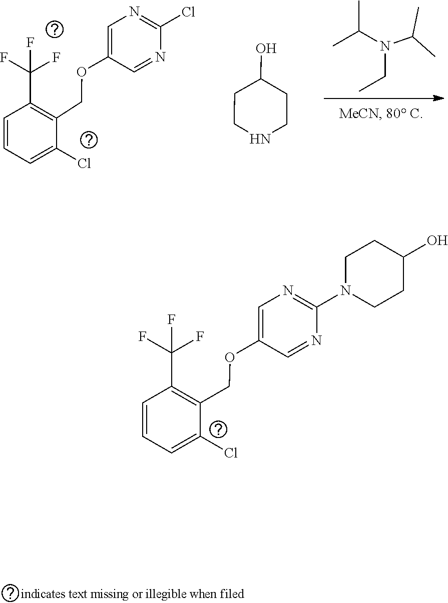

2-chloro-5-{[2-chloro-6-(trifluoromethyl)phenyl] methoxy}pyrimidine, 3564-SB-014-001 (120 mg, 0.37 mmol, 1 eq) was taken up in Acetonitrile (5 mL). 4-HYDROXYPIPERIDINE (45.08 mg, 0 g/mL, 0.45 mmol, 1.2 eq) and N,N-DIISOPROPYLETHYLAMINE (135.39 μL, 0.78 g/mL, 0.82 mmol, 2.2 eq) were added. Rxn was stirred at 80° C. under reflux and N2, for 24 h. A further 30 mg piperidine was added and heating continued overnight. Rxn was partitioned between EtOAc/brine. Organics were dried over MgSO4, filtered and conc in vacuo. The residue was loaded onto isolute. purified by automated flash chromatography (Combiflash 300+, Silica 4 g RediSep column) eluting with 0 to 60% Ethyl Acetate in Heptane F7 and F8 combined and conc in vacuo. The residue was taken up in DCM, then blown down into a submission vial and dried ar 40oC in vacuo overnight to give 1-(5-{[2-chloro-6-(trifluoromethyl)phenyl] methoxy}pyrimidin-2-yl) piperidin-4-ol (65.95 mg, 0.17 mmol, 45.79%) as a clear gum.

### Example 278

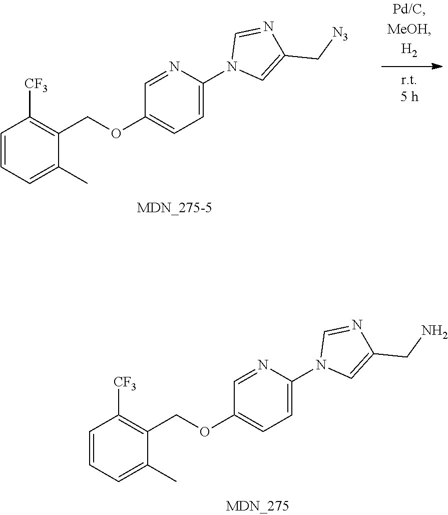 [SMILES: Cc1cccc(C(F)(F)F)c1COc1ccc(-n2cnc(CN=[N+]=[N-])c2)nc1 ; Cc1cccc(C(F)(F)F)c1COc1ccc(-n2cnc(CN)c2)nc1]

To a solution of MDN_275-5 (40 mg, 0.103 mmol) in MeOH (5 mL) was added Pd/C (4 mg). The reaction was stirred at r.t. under H2 atmosphere for 5 h. The reaction mixture was filtered through a pad of Celite and rinsed with MeOH (5 mL×3). The filtrate was concentrated in vacuo and the residue was purified by prep-HPLC (FA) to afford MDN_275 as a white solid (3 mg, 8.0% yield).

### Example 279

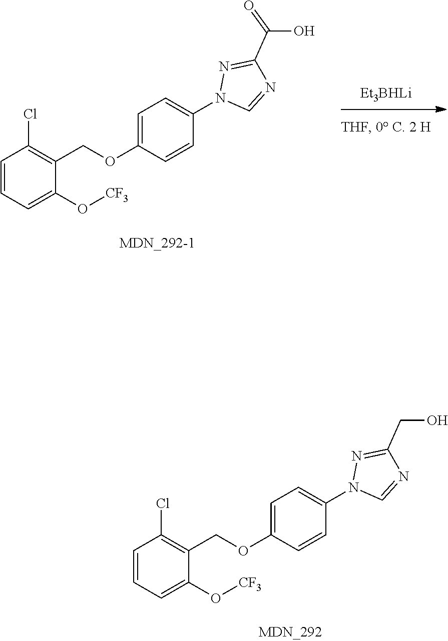

To a solution of MDN_292-1 (0.100 g, 0.234 mmol) in THF (1 mL) was added Et3BHLi (1M in THF, 1.17 mL, 1.17 mmol) under ice bath. The mixture was stirred at ice-bath for 1 h. The mixture was quenched with water, extracted with EA, purified by Pre-TLC (DCM: MeOH=20:1) to give the title compound MDN_292 as a white solid (17.68 mg, 19% yield).

### Example 280

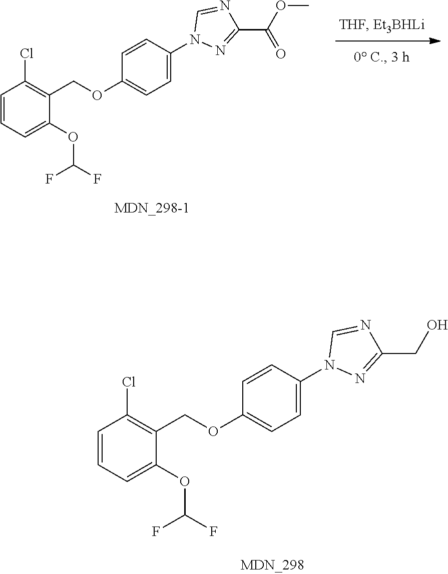 [SMILES: COC(=O)c1ncn(-c2ccc(OCc3c(Cl)cccc3OC(F)F)cc2)n1 ; OCc1ncn(-c2ccc(OCc3c(Cl)cccc3OC(F)F)cc2)n1]

To a solution of MDN_298-1 (100 mg, 0.244 mmol) in THF (2 mL) was added Et3BHLi (1.5 mL, 1.46 mmol) at 0oC. The reaction was stirred at 0° C. for 3 h. The reaction mixture was quenched with H2O (50 mL) and extracted with EA (50 mL×3). The organic layer was dried over Na2SO4, filtered and concentrated in vacuo. The crude product was purified by tablet chromatography (DCM:MeOH=20:1) to afford a white solid (31 mg, 32.7% yield).

### Example 281

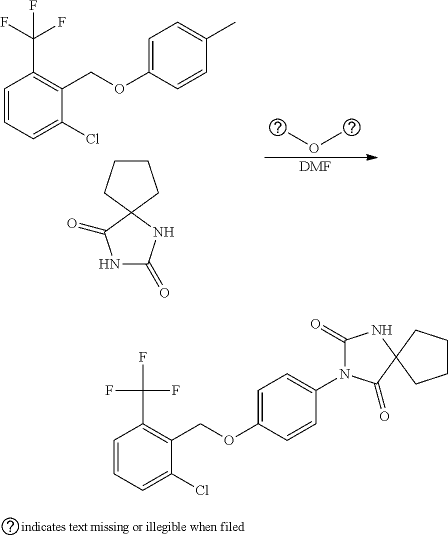

Nitrogen was bubbled through a DMF (1.5 mL) solution of 1-chloro-2-(4-iodophenoxymethyl)-3-(trifluoromethyl)benzene (89 mg, 0.22 mmol, 1 eq) and 1,3-Diazaspiro[4.4]nonane-2,4-dione (49.89 mg, 0.32 mmol, 1.5 eq) in a pressure tube then COPPER (I) OXIDE (30.87 mg, 0.22 mmol, 1 eq) added. The reaction was sealed then heated at 150° C. for 5 h then at 140° C. overnight. The reaction was cooled to rt then diluted with EtOAc and washed with water followed by brine. The organic extract was dried (MgSO4) then the solvent removed in vacuo. The resultant residue was dissolved in DMSO then purified by preparative HPLC automated chromatography (ISCO ACCQ HP150, Prep HPLC Column: Gemini-NX C18 Dimensions: 21 mm×150 mm 5 μM; Sample: 1.40 ml from tube 1) eluting with 5 to 95% ACN/0.1% FA in Water/0.1% FA. Fraction 9 was freeze dried. Gave 3-(4-{[2-chloro-6-(trifluoromethyl)phenyl] methoxy}phenyl)-1,3-diazaspiro[4.4]nonane-2,4-dione (23.85 mg, 0.05 mmol, 25.19%) as a white solid (Purity: >95%).

### Example 282

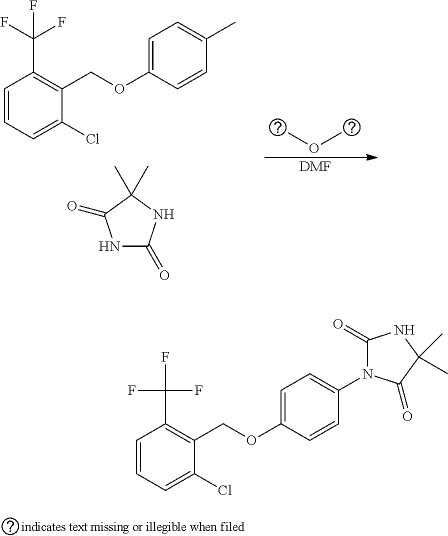

Nitrogen was bubbled through a DMF (1.5 mL) solution of 1-chloro-2-(4-iodophenoxymethyl)-3-(trifluoromethyl)benzene (89 mg, 0.22 mmol, 1 eq) and 5,5-Dimethylhydantoin (41.46 mg, 0.32 mmol, 1.5 eq) in a pressure tube then COPPER (I) OXIDE (30.87 mg, 0.22 mmol, 1 eq) added. The reaction was sealed then heated at 150° C. for 5 h then at 140° C. overnight. The reaction was cooled to rt then diluted with EtOAc and washed with water followed by brine. The organic extract was dried (MgSO4) then the solvent removed in vacuo. The resultant residue was dissolved in DMSO then purified by preparative HPLC automated chromatography (ISCO ACCQ HP150, Sample: 1.40 ml from tube 1; Prep HPLC Column: Gemini-NX C18 Dimensions: 21 mm×150 mm 5 μM) eluting with 5 to 95% ACN/0.1% FA in Water/0.1% FA. Fraction 4 was freeze dried. Gave 3-(4-{[2-chloro-6-(trifluoromethyl)phenyl] methoxy}phenyl)-5,5-dimethylimidazolidine-2,4-dione (25.72 mg, 0.06 mmol, 28.88%) as a white solid (Purity: >95%).

### Example 283

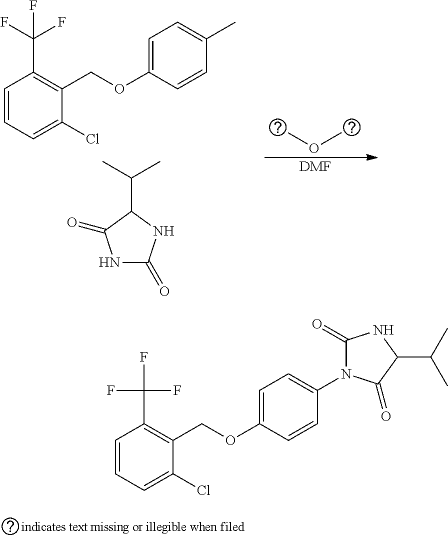

Nitrogen was bubbled through a DMF (1.5 mL) solution of 1-chloro-2-(4-iodophenoxymethyl)-3-(trifluoromethyl)benzene (89 mg, 0.22 mmol, 1 eq) and 5-Isopropylimidazolidine-2,4-dione (46 mg, 0.32 mmol, 1.5 eq) in a pressure tube then COPPER (I) OXIDE (30.87 mg, 0.22 mmol, 1 eq) added. The reaction was sealed then heated at 150° C. for 5 h then at 140° C. overnight. The reaction was cooled to rt then diluted with EtOAc and washed with water followed by brine. The organic extract was dried (MgSO4) then the solvent removed in vacuo. The resultant residue was dissolved in DMSO then purified by preparative HPLC automated chromatography (ISCO ACCQ HP150, Sample: 1.40 ml from tube 1; Prep HPLC Column: Gemini-NX C18 Dimensions: 21 mm×150 mm 5 μM) eluting with 5 to 95% ACN/0.1% FA in Water/0.1% FA. Fraction 11 was freeze dried. Gave 3-(4-{[2-chloro-6-(trifluoromethyl)phenyl] methoxy}phenyl)-5-isopropylimidazolidine-2,4-dione (21.5 mg, 0.05 mmol, 23.35%) as a white solid (Purity: >95%).

### Example 284

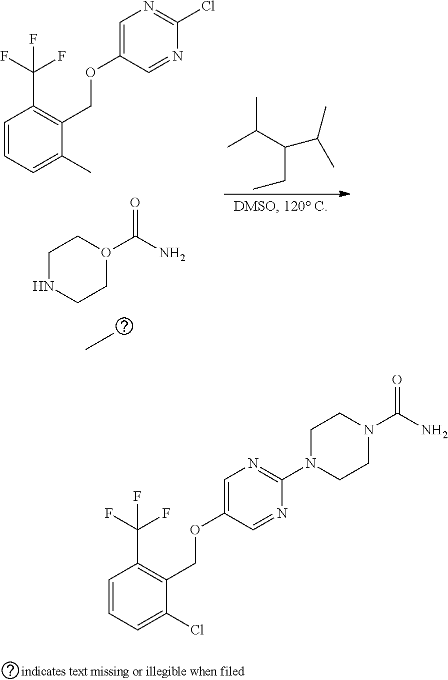

2-chloro-5-{[2-chloro-6-(trifluoromethyl)phenyl] methoxy}pyrimidine (93 mg, 0.29 mmol, 1 eq) was taken up in DMSO (1.5 mL) PIPERAZINE-1-CARBOXYLIC ACID AMIDE HYDROCHLORIDE (52.44 mg, 0.32 mmol, 1.1 eq) and N,NDIISOPROPYLETHYLAMINE (119.23 μL, 0.78 g/mL, 0.72 mmol, 2.5 eq) were added. Rxn sparged with N2. the reaction was microwaved at 120° C. for 1 hour. Rxn about ¼ complete by LC-MS. Small amount of Dess amide product observed. 25 mg of piperazine added. the reaction was microwaved at 120° C. for 1 hour. Small amount of progress seen-decide to isolated. Rxn was run through a syringe filter. purified by preparative HPLC automated chromatography (ISCO ACCQ HP150, Prep HPLC Column: Big Gemini-NX C18 Dimensions: 30 mm×250 mm 5 μM; Sample: 2.30 ml from tube 1) eluting with 5 to 95% ACN/0.1% FA in Water/0.1% FA F22 and 23 combined and frozen in a CO₂/acetone bath, then attached to the freeze drier. 4-(5-{[2-chloro-6-(trifluoromethyl)phenyl] methoxy}pyrimidin-2-yl) piperazine-1-carboxamide (8.04 mg, 0.02 mmol, 6.72%) isolated as a white solid.

### Example 285

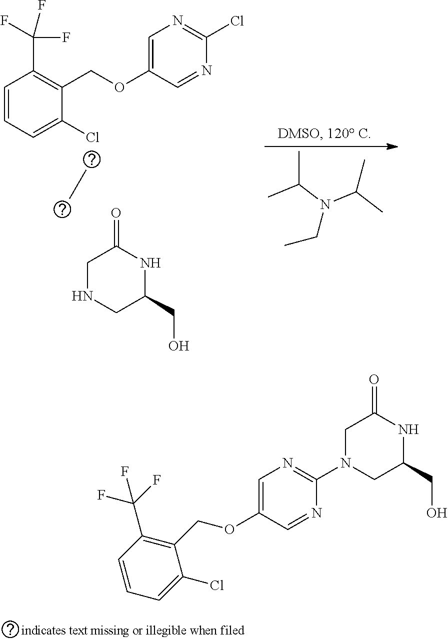

2-chloro-5-{[2-chloro-6-(trifluoromethyl)phenyl] methoxy}pyrimidine, (92 mg, 0.28 mmol, 1 eq) was taken up in DMSO (1.5 mL) (6R)-6-(hydroxymethyl) piperazin-2-one hydrochloride (56.93 mg, 0.34 mmol, 1.2 eq) and N,N-DIISOPROPYLETHYLAMINE, (141.54 μL, 0.78 g/mL, 0.85 mmol, 3 eq) were added. the reaction was microwaved at 120° C. for 1 hour. 25 mg piperazine added, followed by 70 ul DIPEA. the reaction was microwaved at 120° C. for 1 hour. Rxn was run through a syringe filter. purified by preparative HPLC automated chromatography to give (6R)-4-(5-{[2-chloro-6-(trifluoromethyl)phenyl] methoxy}pyrimidin-2-yl)-6-(hydroxymethyl) piperazin-2-one (8.37 mg, 0.02 mmol, 7.05%) as a white solid.

### Example 286

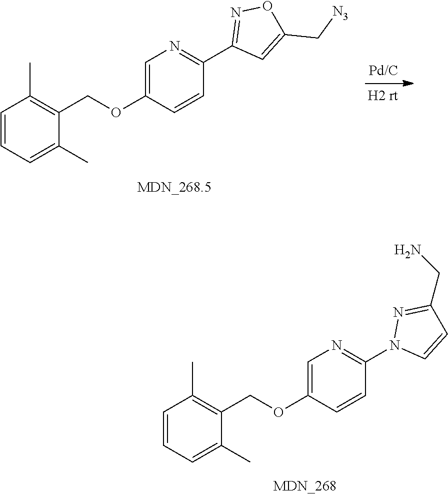 [SMILES: Cc1cccc(C)c1COc1ccc(-c2cc(CN=[N+]=[N-])on2)nc1 ; Cc1cccc(C)c1COc1ccc(-n2ccc(CN)n2)nc1]

To a solution of MDN_268-5 (0.032 g, 0.096 mmol) in MeOH (2 mL) was added Pd/C (10%, 0.010 g) at rt. The mixture was stirred at rt for 2 h under H2. The mixture was filtered, concentrated, purified by chromatography on silica gel (EA: PE=10:1) to give the title compound MDN_268 as a white solid (0.003 g, 11% yield).

### Example 287

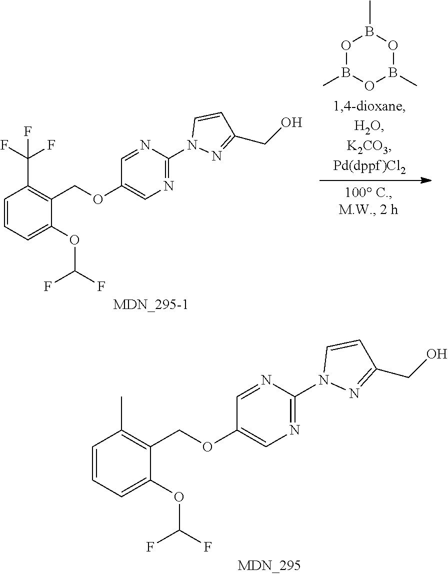

A mixture of MDN_295-1 (0.050 g, 0.117 mmol), 2,4,6-trimethyl-1,3,5,2,4,6-trioxatriborinane (0.5 mL, 0.585 mmol), Pd (dppf) C₁₂ (17 mg, 0.023 mmol) and K₂CO₃ (0.049 g, 0.351 mmol) in 1,4-dioxane/H2O (1 mL/0.1 mL) was stirred at 100oC under Ar in a microwave reactor for 2 h. The reaction mixture was diluted with H2O (30 mL) and extracted with EtOAc (30 mL×3). The organic layer was washed with brine (50 mL), dried over Na2SO4, filtered and concentrated in vacuo. The residue was purified by silica gel column, eluted with DCM: MeOH=20:1 to give the title compound as a yellow solid (15 mg, 50% yield).

### Example 288

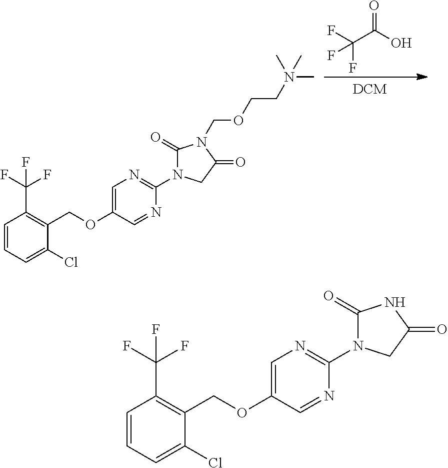

A solution of 1-(5-{[2-chloro-6-(trifluoromethyl)phenyl] methoxy}pyrimidin-2-yl)-3-{[2-(trimethylsilyl) ethoxy]methyl}imidazolidine-2,4-dione (72 mg, 0.14 mmol, 1 eq) and TRIFLUOROACETIC ACID (0.05 mL, 1.53 g/mL, 0.7 mmol, 5 eq) in DCM (2 mL) was stirred at rt overnight. LCMS shows product with MeOH linker still attached The solvent was removed in vacuo then the resultant residue dissolved in THF (2 mL) and 2N (aq) NaOH (0.5 mL) added. The reaction was stirred at rt for ca 3 h. LCMS shows desired product. The reaction was diluted with EtOAc then washed with water followed by brine. The organic extract was dried (MgSO4) then the solvent removed in vacuo. The resultant residue was dissolved in DMSO then purified by preparative HPLC automated chromatography (ISCO ACCQ HP150, Prep HPLC giving 1-(5-{[2-chloro-6-(trifluoromethyl)phenyl] methoxy}pyrimidin-2-yl) imidazolidine-2,4-dione (15 mg, 0.04 mmol, 27.85%) as a white solid (Purity: 90-95%).

### Example 289

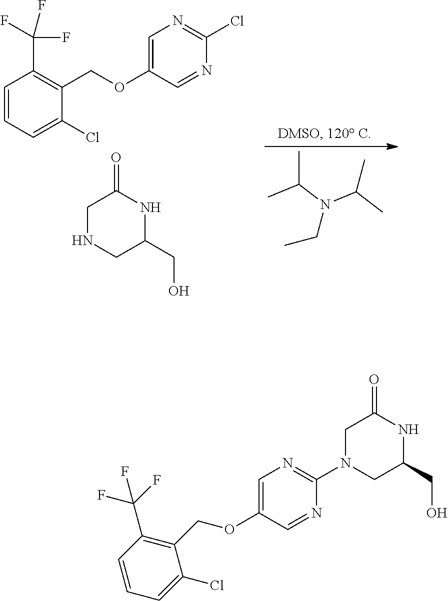

6-(hydroxymethyl) piperazin-2-one (63 mg, 0.48 mmol, 1.74 eq) was taken up in DMSO (2 mL) in a mini sealed tube and 2-chloro-5-{[2-chloro-6-(trifluoromethyl)phenyl] methoxy}pyrimidine, 3564-SB-014-001 (90 mg, 0.28 mmol, 1 eq) was added, followed by N,NDIISOPROPYLETHYLAMINE (101.54 μL, 0.78 g/mL, 0.61 mmol, 2.2 eq) Rxn was degassed, then sealed and heated to 120oC behind a blast shield. After 4 h, rxn was about ⅓ complete by LC-MS. Rxn was re-sealed and heated for a further 4 h on a timer. Rxn was run through a syringe filter. F13 and F14 combined and frozen on a CO₂/acetone bath, then attached to the freeze drier. purified by preparative HPLC automated chromatography (ISCO ACCQ HP150, Prep HPLC Column: Big Gemini-NX C18 Dimensions: 30 mm×250 mm 5 M; Sample: 2.20 ml from tube 1) eluting with 5 to 95% ACN/0.1% FA in Water/0.1% FA to give 4-(5-{[2-chloro-6-(trifluoromethyl)phenyl] methoxy}pyrimidin-2-yl)-6-(hydroxymethyl) piperazin-2-one (24.83 mg, 0.06 mmol, 21.39%) as a white solid.

### Example 290

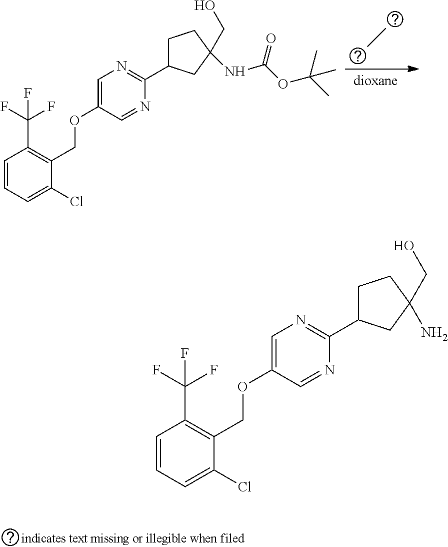

tert-butyl N-[1-(5-{[2-chloro-6-(trifluoromethyl)phenyl] methoxy}pyrimidin-2-yl)-3-(hydroxymethyl) pyrrolidin-3-yl]carbamate, 3564-SB-033-001 (69 mg, 0.14 mmol, 1 eq) was taken up in 1,4-Dioxane (1 mL) and hydrochloric acid (3.09 mL, 4M in, 12.35 mmol, 90 eq) (in dioxane) was added. Rxn was stirred at rt. After 2 h, rxn complete by LC-MS. Rxn was conc in vacuo, then taken up in DMSO and filtered. F18 and 19 combined and frozen on a CO₂/acetone bath, then attached to the freeze drier giving [3-amino-1-(5-{[2-chloro-6-(trifluoromethyl)phenyl] methoxy}pyrimidin-2-yl) pyrrolidin-3-yl]methanol (22.91 mg, 0.06 mmol, 41.46%) 3 as a white solid.

### Example 291

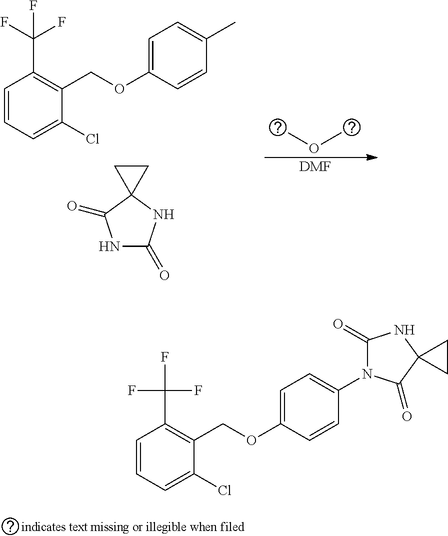

Nitrogen was bubbled through a DMF (1.5 mL) solution of 1-chloro-2-(4-iodophenoxymethyl)-3-(trifluoromethyl)benzene (89 mg, 0.22 mmol, 1 eq) and 4,6-diazaspiro[2.4]heptane-5,7-dione (40.81 mg, 0.32 mmol, 1.5 eq) in a pressure tube then COPPER (I) OXIDE (30.87 mg, 0.22 mmol, 1 eq) was added. The reaction was sealed then heated to 150oC, where it was maintained overnight. LCMS shows the reaction has gone to completion. The reaction was cooled to rt then diluted with EtOAc and washed with water followed by brine. The organic extract was dried (MgSO4) then the solvent removed in vacuo. The resultant residue was dissolved in DMSO, filtered then purified by preparative HPLC automated chromatography (ISCO ACCQ HP150, Prep HPLC Column: Gemini-NX C18 Dimensions: 21 mm×150 mm 5 μM; Sample: 1.30 ml from tube 1) eluting with 5 to 95% ACN/0.1% FA in Water/0.1% FA. Fractions 6 & 7 were combined then freeze dried giving 6-(4-{[2-chloro-6-(trifluoromethyl)phenyl] methoxy}phenyl)-4,6-diazaspiro[2.4]heptane-5,7-dione (23.24 mg, 0.06 mmol, 26.23%) as a white solid (Purity: 90-95%).

### Example 292

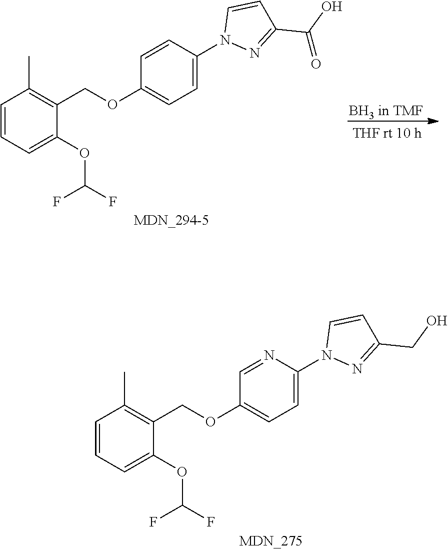 [SMILES: Cc1cccc(OC(F)F)c1COc1ccc(-n2ccc(C(=O)O)n2)cc1 ; Cc1cccc(OC(F)F)c1COc1ccc(-n2ccc(CO)n2)nc1]

To a mixture of MDN_294-5 (138 mg, 0.37 mmol) in THF was added BH3 in THF (1M, 1.84 mL, 1.84 mmol) dropwised under ice cooling bath. The mixture was stirred at rt overnight. The mixture was quenched with MeOH (10 mL), extracted with EA and concentrated to give the residue, which was purified by chromatography on silica gel to give the title compound MDN_294 as a white solid (18 mg, 15% yield).

### Example 293

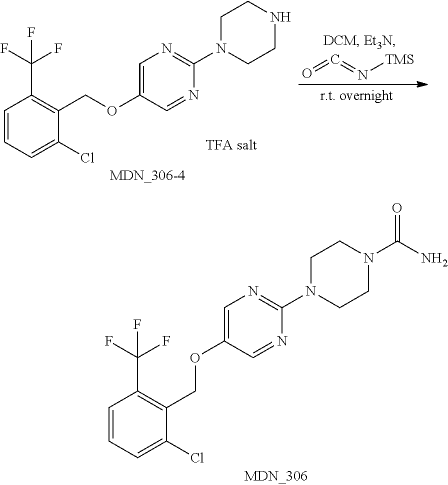

To a mixture of MDN_306-4 (0.142 g, 0.382 mmol) and Et3N (0.116 g, 1.15 mmol) in DCM (3 mL) was added isocyanatotrimethylsilane (0.220 g, 1.91 mmol) at 0oC. The reaction mixture was stirred at r.t. overnight. The reaction mixture was diluted with H2O (30 mL) and extracted with DCM (30 mL×3). The organic phase was dried over Na2SO4, filtered and concentrated in vacuo. The crude product was purified by silica gel column (DCM/MeOH=10/1) to give the title compound as yellow oil (0.030 g, 19% yield).

### Example 295

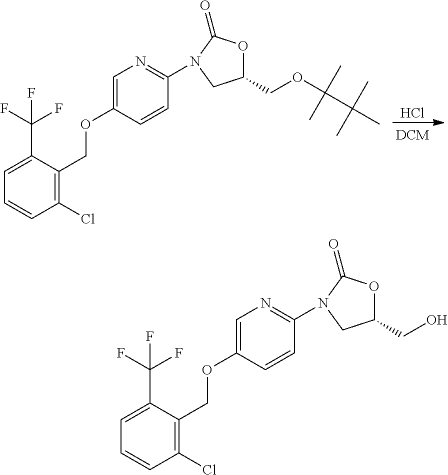 [SMILES: CC(C)(C)C(C)(C)OC[C@@H]1CN(c2ccc(OCc3c(Cl)cccc3C(F)(F)F)cn2)C(=O)O1 ; Cl ; O=C1O[C@H](CO)CN1c1ccc(OCc2c(Cl)cccc2C(F)(F)F)cn1]

HYDROCHLORIC ACID (4N in 1,4-dioxane) (0.21 mL, 4M in, 0.85 mmol, 5 eq) was added to a stirred solution of (5S)-5-{[(tertbutyldimethylsilyl)oxy] methyl}-3-(5-{[2-chloro-6-(trifluoromethyl)phenyl] methoxy}pyridin-2-yl)-1,3-oxazolidin-2-one (88 mg, 0.17 mmol, 1 eq) in DCM (2 mL). The reaction was stirred at rt for ca 3 h. The solvent was removed in vacuo then the resultant residue dissolved in DMSO, filtered and purified by preparative HPLC automated chromatography (ISCO ACCQ HP150, Prep HPLC Column: Gemini-NX C18 Dimensions: 21 mm×150 mm 5 μM; Sample: 1.30 ml from tube 1) eluting with 5 to 95% ACN/0.1% FA in Water/0.1% FA. Fractions 2 & 3 were combined then freeze dried. Gave (5S)-3-(5-{[2-chloro-6-(trifluoromethyl)phenyl] methoxy}pyridin-2-yl)-5-(hydroxymethyl)-1,3-oxazolidin-2-one (46.13 mg, 0.11 mmol, 67.29%) as a white Purity: >98%).

### Example 296

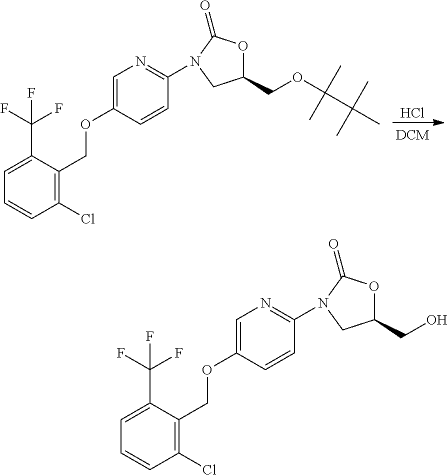 [SMILES: CC(C)(C)C(C)(C)OC[C@H]1CN(c2ccc(OCc3c(Cl)cccc3C(F)(F)F)cn2)C(=O)O1 ; Cl ; O=C1O[C@@H](CO)CN1c1ccc(OCc2c(Cl)cccc2C(F)(F)F)cn1]

HYDROCHLORIC ACID (4N in 1,4-dioxane) (0.21 mL, 4M in, 0.85 mmol, 5 eq) was added to a stirred solution of (5S)-5-{[(tertbutyldimethylsilyl)oxy] methyl}-3-(5-{[2-chloro-6-(trifluoromethyl)phenyl] methoxy}pyridin-2-yl)-1,3-oxazolidin-2-one (88 mg, 0.17 mmol, 1 eq) in DCM (2 mL). The reaction was stirred at rt for ca 3 h. The solvent was removed in vacuo then the resultant residue dissolved in DMSO, filtered and purified by preparative HPLC automated chromatography (ISCO ACCQ HP150, Prep HPLC Column: Gemini-NX C18 Dimensions: 21 mm×150 mm 5 μM; Sample: 1.30 ml from tube 1) eluting with 5 to 95% ACN/0.1% FA in Water/0.1% FA. Fractions 2 & 3 were combined then freeze dried. Gave (5S)-3-(5-{[2-chloro-6-(trifluoromethyl)phenyl] methoxy}pyridin-2-yl)-5-(hydroxymethyl)-1,3-oxazolidin-2-one (46.13 mg, 0.11 mmol, 67.29%) as a white solid (Purity: >98%).

### Example 297

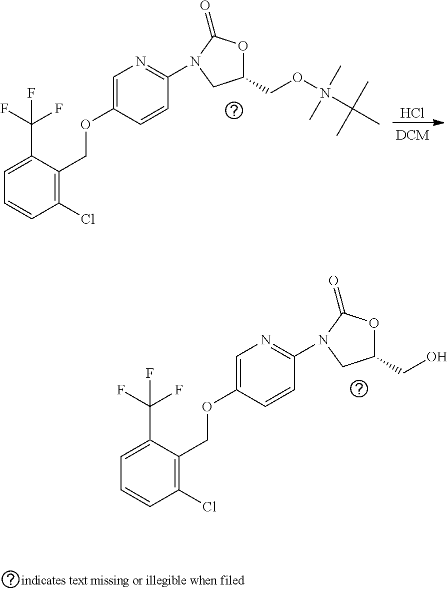

HYDROCHLORIC ACID (4N in HCl) (0.22 mL, 4M in, 0.87 mmol, 5 eq) was added to a stirred solution of (5R)-5-{[(tertbutyldimethylsilyl)oxy] methyl}-3-(5-{[2-chloro-6-(trifluoromethyl)phenyl] methoxy}pyridin-2-yl)-1,3-oxazolidin-2-one (90 mg, 0.17 mmol, 1 eq) in DCM (2 mL). The reaction was stirred at rt for ca 3 h. The solvent was removed in vacuo then the resultant residue dissolved in DMSO, filtered and purified by preparative HPLC automated chromatography (ISCO ACCQ HP150, Prep HPLC Column: Gemini-NX C18 Dimensions: 21 mm×150 mm 5 μM; Sample: 1.30 ml from tube 1) eluting with 5 to 95% ACN/0.1% FA in Water/0.1% FA. Fractions 10 & 11 were combined then freeze dried. Gave (5R)-3-(5-{[2-chloro-6-(trifluoromethyl)phenyl] methoxy}pyridin-2-yl)-5-(hydroxymethyl)-1,3-oxazolidin-2-one (41.78 mg, 0.1 mmol, 59.59%) as a white solid (Purity: >98%).

### Example 298

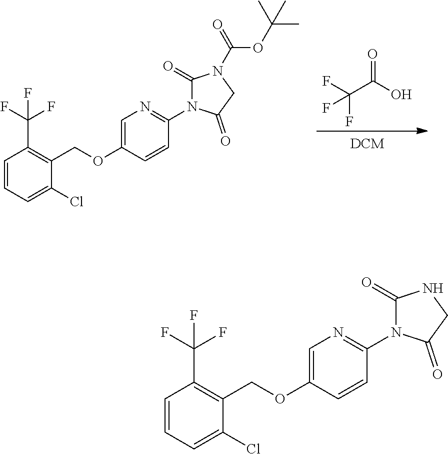 [SMILES: CC(C)(C)OC(=O)N1CC(=O)N(c2ccc(OCc3c(Cl)cccc3C(F)(F)F)cn2)C1=O ; O=C(O)C(F)(F)F ; O=C1CNC(=O)N1c1ccc(OCc2c(Cl)cccc2C(F)(F)F)cn1]

TRIFLUOROACETIC ACID (0.03 mL, 1.53 g/mL, 0.4 mmol, 5 eq) was added to a stirred solution of tert-butyl 3-(5-{[2-chloro-6-(trifluoromethyl)phenyl] methoxy}pyridin-2-yl)-2,4-dioxoimidazolidine-1-carboxylate (38.8 mg, 0.08 mmol, 1 eq) in DCM (3 mL). The reaction was stirred at rt for ca 3 h. The solvent was removed in vacuo then the resultant residue dissolved in DMSO, filtered and purified by preparative HPLC automated chromatography (ISCO ACCQ HP150, Prep HPLC Column: Gemini-NX C18 Dimensions: 21 mm×150 mm 5 μM; Sample: 1.30 ml from tube 1) eluting with 5 to 95% ACN/0.1% FA in Water/0.1% FA. Fraction 2 was freeze dried giving 3-(5-{[2-chloro-6-(trifluoromethyl)phenyl] methoxy}pyridin2-yl) imidazolidine-2,4-dione (16.13 mg, 0.04 mmol, 52.36%) as a white solid (Purity: >95%).

### Example 299

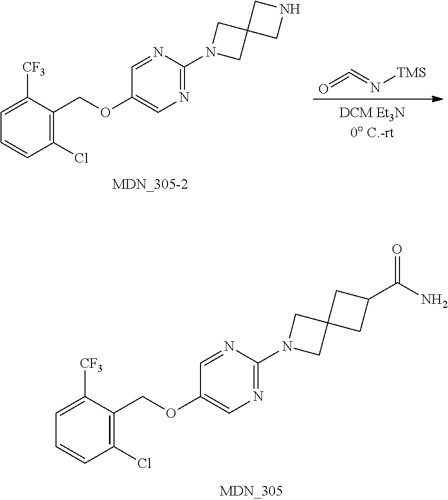 [SMILES: FC(F)(F)c1cccc(Cl)c1COc1cnc(N2CC3(CNC3)C2)nc1 ; C[Si](C)(C)N=C=O ; NC(=O)C1CC2(C1)CN(c1ncc(OCc3c(Cl)cccc3C(F)(F)F)cn1)C2]

To a solution of MDN_305-2 (0.057 g, 0.148 mmol) in DCM (2 mL) was added Et3N (0.044 g, 0.444 mmol) and SM2 (0.085 g, 0.74 mmol) at 0oC. The mixture was stirred at rt for 2 h. The mixture was quenched with water, extracted with DCM. The mixture was purified by Pre-TLC (DCM:MeOH=10:1) to give the title compound MDN_305 as a white solid (0.024 g, 38% yield).

### Example 300

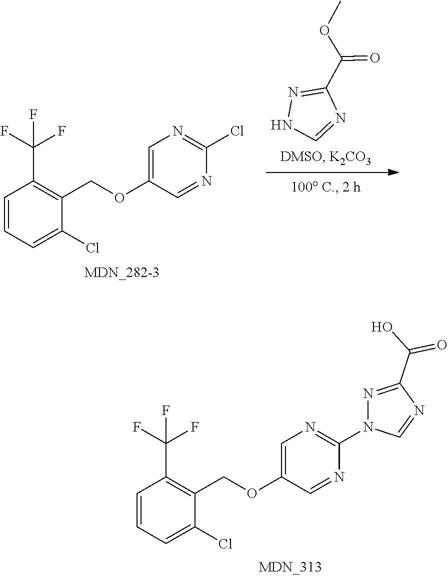

To a solution of 2-chloro-5-((2-chloro-6-(trifluoromethyl)benzyl)oxy) pyrimidine (300 mg, 0.929 mmol) in DMSO (2 mL) were added methyl 1H-1,2,4-triazole-3-carboxylate (118 mg, 0.929 mmol) and K₂CO₃ (257 mg, 1.85 mmol). The reaction was stirred at 100oC for 2 h. The reaction mixture was diluted with H2O (30 mL) and washed with EtOAc (30 mL×2). The aqueous phase was acidified by 1NHCl to pH 2 and extracted with EtOAc (30 mL×3). The organic layer was dried over Na2SO4, filtered and concentrated in vacuo. The residue was purified by flash chromatography (ACN: H2O(FA)=0%˜100%) to afford MDN_313 as a white solid (33.5 mg, 9.0% yield).

### Example 301

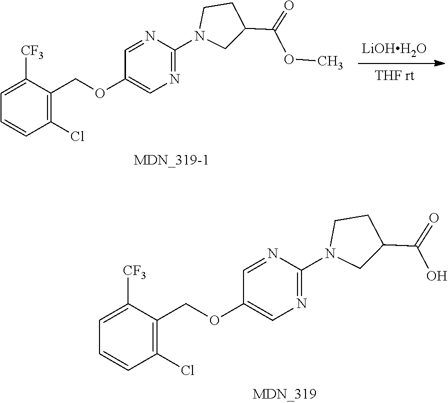

To a solution of MDN_319-1 (0.149 g, 0.358 mmol) and in THF (2 mL) was added LiOH.H2O (0.075 g, 1.79 mmol) in water (0.447 mL) at rt. The mixture was stirred at rt for 2 h. The mixture was washed with ether, water phase was adjusted PH=2, filtered. The solid was washed with DMSO, filtered again, washed with water, dried in vacuo to get the title compound MDN_319 as a white solid (0.098 g, 68% yield).

### Example 302

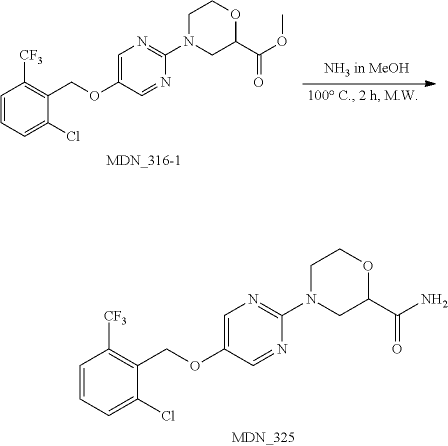 [SMILES: COC(=O)C1CN(c2ncc(OCc3c(Cl)cccc3C(F)(F)F)cn2)CCO1 ; NC(=O)C1CN(c2ncc(OCc3c(Cl)cccc3C(F)(F)F)cn2)CCO1]

A mixture of MDN_316-1 (50 mg, 0.116 mmol) and NH₃-MeOH (7.0M, 5 mL) was sealed and stirred at 100oC in M.W. for 2 h. The reaction mixture was concentrated in vacuo. The crude product was purified by tablet chromatography (PE:EA=1:1) to afford title compound (31.5 mg, 65.2% yield).

### Example 305

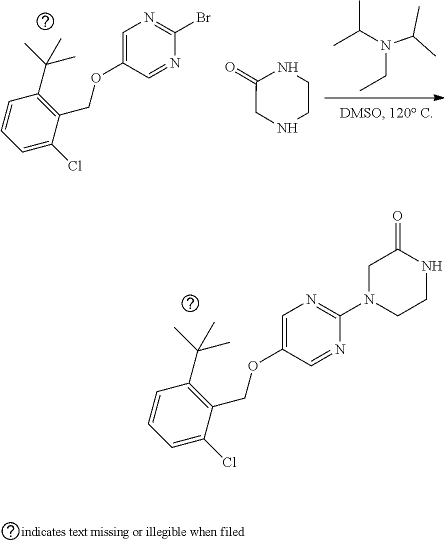

2-bromo-5-{[2-chloro-6-(trifluoromethyl)phenyl] methoxy}pyrimidine (161 mg, 0.44 mmol, 1 eq) was taken up in DMSO (4 mL) and added to a sealed tube. piperazin-2-one (65.78 mg, 0.66 mmol, 1.5 eq) and N,N-DIISOPROPYLETHYLAMINE (145.16 μL, 0.78 g/mL, 0.88 mmol, 2 eq) were added. Rxn was sealed and heated to 120oC behind a blast shield. After 18 h, rxn complete by LC-MS. Rxn was partitioned between EtOAc/H2O. Organics were dried over MgSO4, filtered and conc in vacuo. The residue was taken up in 1.5 ml DMSO and filtered. purified by preparative HPLC automated chromatography (ISCO ACCQ HP150, Prep HPLC Column: Big Gemini-NX C18 Dimensions: 30 mm×250 mm 5 M; Sample: 2.00 ml from tube 1) eluting with 5 to 95% ACN/0.1% FA in Water/0.1% FA. F20 to F22 combined and frozen on a CO₂/acetone bath, then attached to the freeze drier to give 4-(5-{[2-chloro-6-(trifluoromethyl)phenyl] methoxy}pyrimidin-2-yl) piperazin-2-one (74.43 mg, 0.19 mmol, 43.93%) as white solid.

### Example 306

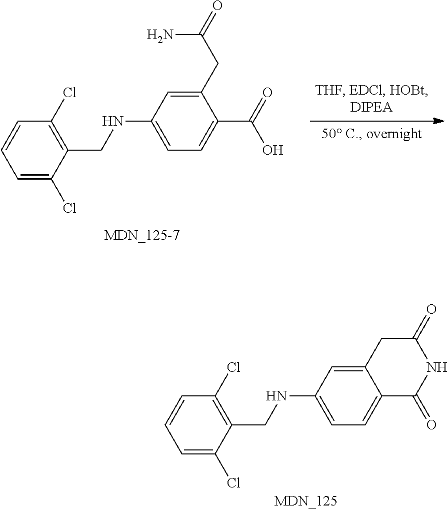 [SMILES: NC(=O)Cc1cc(NCc2c(Cl)cccc2Cl)ccc1C(=O)O ; O=C1Cc2cc(NCc3c(Cl)cccc3Cl)ccc2C(=O)N1]

To a solution of MDN_125-7 (66 mg, 0.187 mmol) in THF (1 mL) were added EDCI (43 mg, 0.224 mmol), HOBt (30 mg, 0.224 mmol) and DIPEA (73 mg, 0.561 mmol). The reaction was stirred at 50oC overnight. The reaction mixture was diluted with H2O (30 mL) and extracted with EtOAc (30 mL×3). The organic layer was dried over Na2SO4, filtered and concentrated in vacuo. The residue was purified by flash chromatography (ACN: H2O=0%˜100%) to afford the title compound (6.61 mg, 10.5% yield) as a white solid.

### Example 307

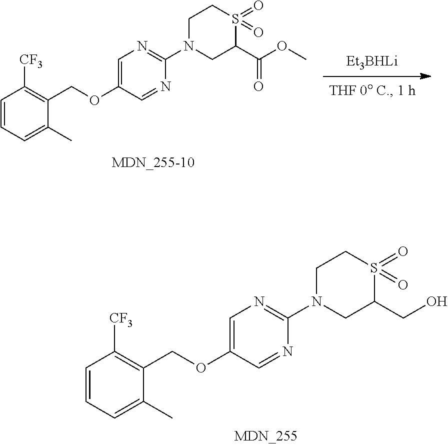

To a solution of MDN_255-10 (60 mg, 0.13 mmol) in THF (1 mL) was added Et3BHLi (1M in THF, 0.65 mL, 0.65 mmol) dropwised under ice cooling. The mixture was stirred under ice cooling for 1 h. The mixture was quenched with H2O, extracted with EA, dried over Na2SO4, purified by pre-HPLC to give the title compound MDN_255 as a white solid (10 mg, 18% yield).

### Example 308

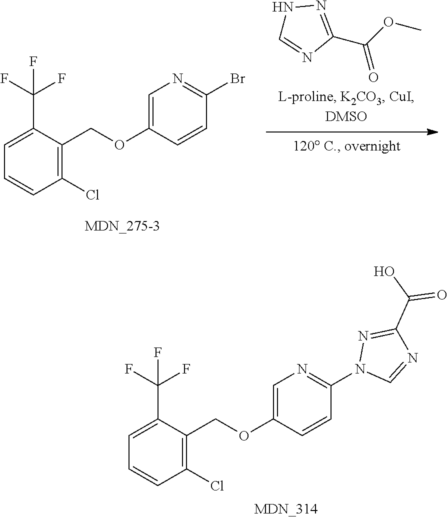

To a mixture of MDN_275-3 (200 mg, 0.545 mmol) in DMSO (2 mL) was added methyl 1H-1,2,4-triazole-3-carboxylate (139 mg, 1.09 mmol), L-proline (13 mg, 0.011 mmol), CuI (10 mg, 0.055 mmol) and K₂CO₃ (226 mg, 1.64 mmol). The reaction mixture was stirred at 120oC overnight. The reaction mixture was diluted with H2O (30 mL) and extracted with EtOAc (30 mL×3). The organic phase was dried over Na2SO4, concentrated and the crude product was purified by silica gel column give the title compound as a white solid (10 mg, 5% yield).

### Example 309

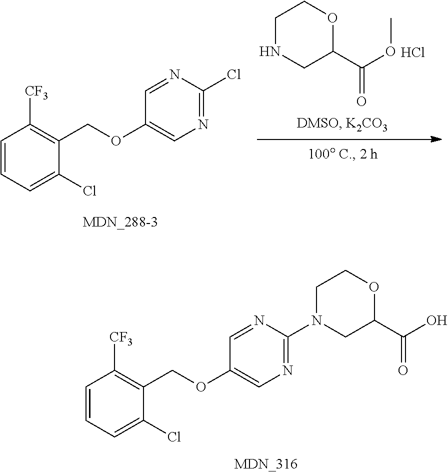

To a solution of MDN_288-3 (200 mg, 1.10 mmol) in DMSO (3 mL) were added K2CO3 (457 mg, 3.30 mmol) and methyl morpholine-2-carboxylate hydrochloride (356 mg, 1.10 mmol). The reaction was stirred at 100oC for 2 h. The reaction mixture was diluted with H2O (30 mL) and extracted with EtOAc (30 mL×3). The organic layer was dried over Na2SO4, filtered and concentrated in vacuo. The crude product was purified by prep-HPLC (FA) and lyophilized to afford the title compound (6.45 mg, 1.40% yield) as a white solid.

### Example 310

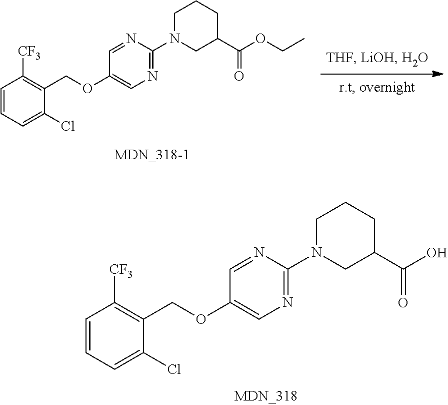 [SMILES: CCOC(=O)C1CCCN(c2ncc(OCc3c(Cl)cccc3C(F)(F)F)cn2)C1 ; O=C(O)C1CCCN(c2ncc(OCc3c(Cl)cccc3C(F)(F)F)cn2)C1]

A mixture of MDN_318-1 (50 mg, 0.12 mmol) in THF (0.5 mL) was added LiOH H2O (47.27 mg, 1.2 mmol) in H2O (0.5 mL). The mixture was stirred at rt overnight. The mixture was concentrated, diluted with water, adjusted PH=2, extracted with EA and concentrated to give the residue, which was purified by chromatography on silica gel (EA/PE=0-50%) to give the title compound MDN_318 as a white solid (8 mg, 20% yield).

### Example 311

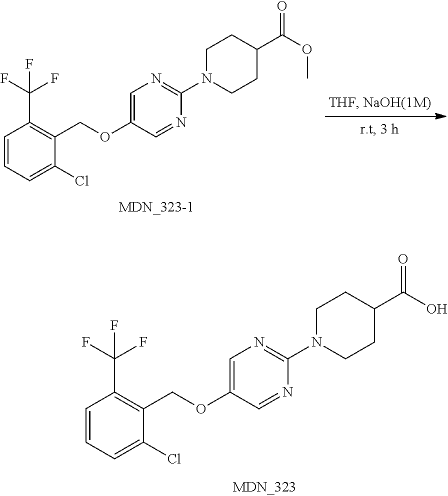 [SMILES: COC(=O)C1CCN(c2ncc(OCc3c(Cl)cccc3C(F)(F)F)cn2)CC1 ; O=C(O)C1CCN(c2ncc(OCc3c(Cl)cccc3C(F)(F)F)cn2)CC1]

To a solution of MDN_323-1 (85 mg, 0.198 mmol) in THF (2 mL) was added aqueous NaOH (1.00 mol/L, 1.5 mL). The reaction was stirred at room temperature for 3 h. The reaction mixture was diluted with H2O (30 mL) and extracted with EA (30 mL×3). The organic layer was dried over Na2SO4, filtered and concentrated in vacuo. The crude product was purified by tablet chromatography (PE:EA=10:1) to afford title compound (32.8 mg, 39.9% yield) as a white solid.

### Example 312

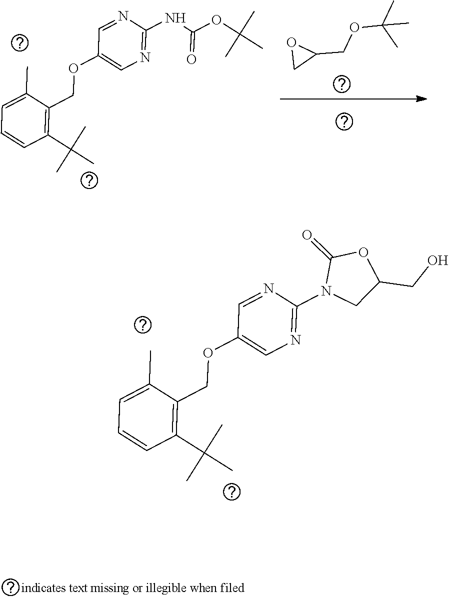

To a solution of the above starting compound (120 mg, 0.3 mmol, 1 eq) in DMF (5 mL) was added tert-Butyl glycidyl ether (63.29 μL, 0.92 g/mL, 0.45 mmol, 1.5 eq) followed by cesium carbonate (145.25 mg, 0.45 mmol, 1.5 eq) Stirred at 100 C for 2 hours. The reaction mixture was diluted with EtOAc (10 mL), filtered and conc in vacuum to give a red/brown oil 5-(tert-butoxymethyl)-3-(5-{[2-chloro-6-(trifluoromethyl)phenyl] methoxy}pyrimidin-2-yl)-1,3-oxazolidin-2-one (104 mg, 0.23 mmol, 76.1%). The recovered compound was dissolved into DCM (2 mL) and trifluoroacetic acid (0.5 mL) was added at rt. Stirred at rt for 3 hours. The reaction mixture was conc in vacuum on the V10, diluted with DMSO (4 mL) and purified by preparative HPLC automated chromatography (ISCO ACCQ HP150, Prep HPLC Column: Big Gemini-NX C18 Dimensions: 30 mm×250 mm 5 μM; Sample: 2.10 ml from tube 1) eluting with 5 to 95% ACN/0.1% FA in Water/0.1% FA Fractions 8-9 and 31-32 to give: 3-(5-{[2-chloro-6-(trifluoromethyl)phenyl] methoxy}pyrimidin-2-yl)-5-(hydroxymethyl)-1,3-oxazolidin-2-one (21 mg, 0.05 mmol, 17.5%) as a white solid (Purity: 90-95%)

### Example 313

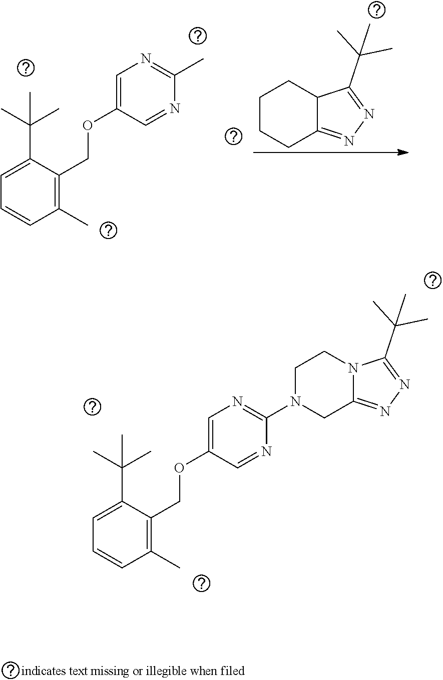 [SMILES: Cc1ncc(OCc2c(C)cccc2C(C)(C)C)cn1 ; CC(C)(C)C1=NN=C2CCCCC21 ; Cc1cccc(C(C)(C)C)c1COc1cnc(N2CCn3c(nnc3C(C)(C)C)C2)nc1]

To a solution of the above starting compound (190 mg, 0.59 mmol, 1 eq) in ethanol (4 mL) was added 3-(trifluoromethyl)-5,6,7,8-tetrahydro-[1,2,4]triazolo[4,3-a]pyrazine1 (112.99 mg, 0.59 mmol, 1 eq) and Hunigs Base (0.2 mL, 1.18 mmol, 2 eq). Stirred on a seal tube at 120 C for 16 hours. The reaction mixture was cooled to RT, diluted with EtoAc (50 mL) conc in vacuum, washed with NH4Cl, brine, dried over MgSO4 and conc in vacuum. purified by automated flash chromatography (Combiflash Rf, Silica 12 g RediSep column) eluting with 0 to 100% Ethyl Acetate in Heptane fractions 12-16 to give: 5-{[2-chloro-6-(trifluoromethyl)phenyl] methoxy}-2-[3-(trifluoromethyl)-5H,6H,8H-[1,2,4]triazolo[4,3-alpyrazin-7-yl]pyrimidine (7.83 mg, 0.02 mmol, 2.78%) as a white solid (Purity: 90-95%)

### Example 314

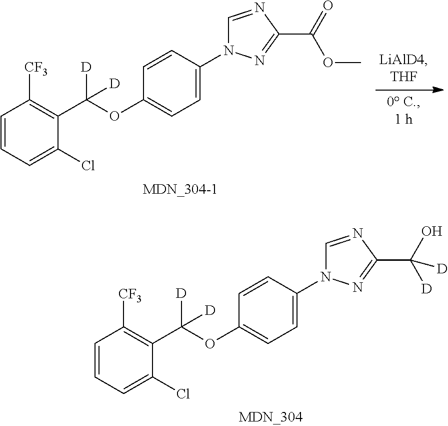 [SMILES: [2H]C([2H])(Oc1ccc(-n2cnc(C(=O)OC)n2)cc1)c1c(Cl)cccc1C(F)(F)F ; [2H]C([2H])(O)c1ncn(-c2ccc(OC([2H])([2H])c3c(Cl)cccc3C(F)(F)F)cc2)n1]

To a solution of MDN_304-1 (80 mg, 0.193 mmol) in THF (2 mL) was added LiAID4 (40.6 mg, 0.967 mmol) at 0oC. The reaction was stirred at 0oC for 1 h. The reaction mixture was quenched with H2O and extracted with EA (30 mL×3). The organic layer was dried over Na2SO4, filtered and concentrated in vacuo. The crude product was purified by tablet chromatography (DCM:MeOH=10:1) to afford the title compound (16.3 mg, 21.7% yield) as a white solid.

### Example 315

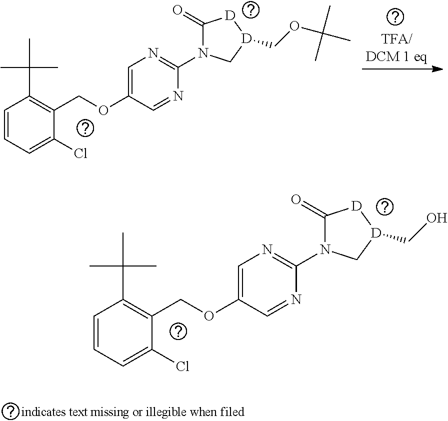

HYDROCHLORIC ACID (4N in 1,4-dioxane) (0.04 mL, 4M in, 0.16 mmol, 5 eq) was added to a stirred solution of (4R)-4-{[(tertbutyldimethylsilyl)oxy] methyl}-1-(5-{[2-chloro-6-(trifluoromethyl)phenyl] methoxy}pyrimidin-2-yl) imidazolidin-2-one (17 mg, 0.03 mmol, 1 eq) in DCM (1 mL). The reaction was stirred at rt for ca 2 h. LCMS shows product+unknown. The solvent was removed in vacuo then the resultant residue dissolved in DMSO, filtered and purified by preparative HPLC automated chromatography (ISCO ACCQ HP150, Prep HPLC Column: Gemini-NX C18 Dimensions: 21 mm×150 mm 5 μM; Sample: 1.30 ml from tube 1). eluting with 5 to 95% ACN/0.1% FA in Water/0.1% FA Fraction 2 was freeze dried giving (4R)-1-(5-{[2-chloro-6-(trifluoromethyl)phenyl] methoxy}pyrimidin-2-yl)-4-(hydroxymethyl) imidazolidin-2-one (4.62 mg, 0.01 mmol, 34.89%) as a colourless glass (Purity: >95%)

### Example 316

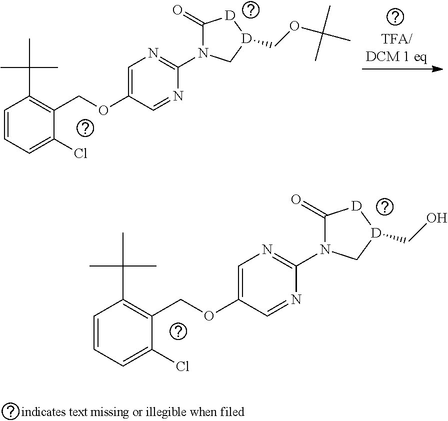

To a solution of the above starting compound (195 mg, 0.42 mmol, 1 eq) in DCM (3 mL) was added trifluoroacetic acid (0.5 mL) and stirred at RT for 4 h at rt.: The reaction mixture was conc in vacuum, diluted with DMSO (4 mL) and purified by preparative HPLC automated chromatography (ISCO ACCQ HP150, Sample: 2.00 ml from tube 1; Prep HPLC Column: Big Gemini-NX C18 Dimensions: 30 mm×250 mm 5 μM) eluting with 5 to 95% ACN/0.1% FA in Water/0.1% FA fractions 7-8 and 31-32 to give: (5S)-3-(5-{[2-chloro-6-(trifluoromethyl)phenyl] methoxy}pyrimidin-2-yl)-5-(hydroxymethyl)-1,3-oxazolidin-2-one (104 mg, 0.26 mmol, 60.74%) as a white solid (Purity: >98%).

### Example 317

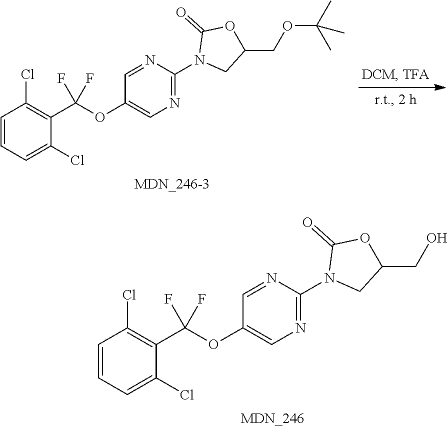 [SMILES: CC(C)(C)OCC1CN(c2ncc(OC(F)(F)c3c(Cl)cccc3Cl)cn2)C(=O)O1 ; O=C1OC(CO)CN1c1ncc(OC(F)(F)c2c(Cl)cccc2Cl)cn1]

To a solution of MDN_246-3 (30 mg, 0.065 mmol) in DCM (3 mL) was added TFA (1 mL). The reaction was stirred at room temperature overnight. The reaction mixture was concentrated in vacuo to remove volatiles. The residue was diluted with H2O (10 mL), neutralized with NaHCO3 to pH 8 and extracted with EtOAc (30 mL×3). The organic layer was dried over Na2SO4, filtered and concentrated in vacuo. The crude product was purified by prep-TLC (EA: PE=1:1) to afford a white solid (27 mg, 69.1% yield).

### Example 319

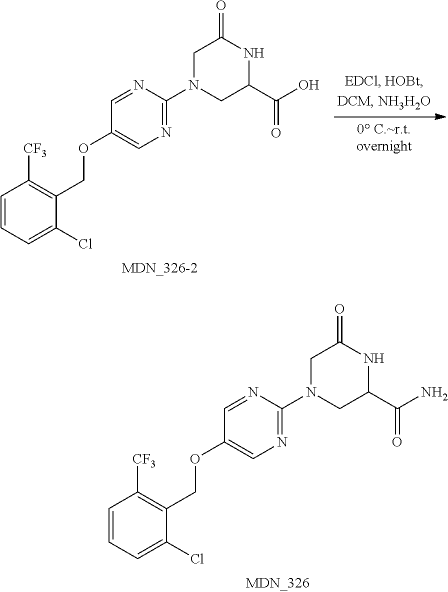 [SMILES: O=C1CN(c2ncc(OCc3c(Cl)cccc3C(F)(F)F)cn2)CC(C(=O)O)N1 ; NC(=O)C1CN(c2ncc(OCc3c(Cl)cccc3C(F)(F)F)cn2)CC(=O)N1]

To a solution of MDN_326-2 (98 mg, 0.227 mmol) in DCM (1 mL) were added EDCI (52.3 mg, 0.273 mmol) and HOBT (36.9 mg, 0.273 mmol) at 0oC. The reaction was stirred at R.T. for 0.5 h. The mixture was cooled to 0° C. and NH₃H2O (1.5 mL) was added. The reaction was stirred at room temperature overnight. The reaction mixture diluted with H2O (30 mL) and extracted with EtOAc (30 mL×3). The organic layer was dried over Na2SO4, filtered and concentrated in vacuo. The crude product was purified by prep-TLC (DCM: MeOH=10:1) and prep-HPLC (NH4HCO3), then lyophilized to afford the title compound (11.21 mg, 11.5% yield).

### Example 320

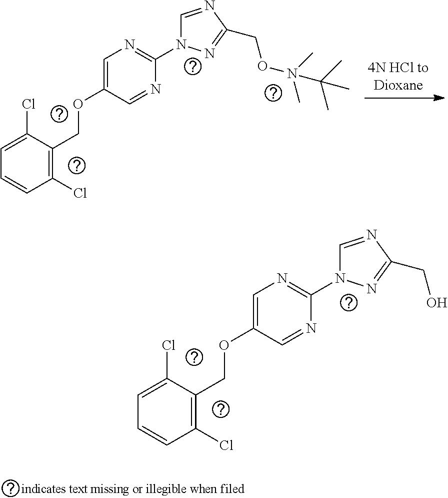

A solution of the above starting compound (200 mg, 0.43 mmol, 1 eq) in HCl (4M in Dioxane) (2 mL) was stirred at rt for 2 hours. Work-up: The reaction mixture was conc in vacuum on the genevac, diluted dith DMSO (4 mL) and purified by preparative HPLC automated chromatography (ISCO ACCQ HP150, Prep HPLC Column: Big Gemini-NX C18 Dimensions: 30 mm×250 mm 5 μM; Sample: 2.10 ml from tube 1) eluting with 5 to 95% ACN/0.1% FA in Water/0.1% FA fractions 8-10 and 21-23 to give (1-{5-[(2,6-dichlorophenyl) methoxy]pyrimidin-2-yl}-1,2,4-triazol-3-yl) methanol (71 mg, 0.2 mmol, 47.02%) white solid (Purity: >98%).

### Example 321

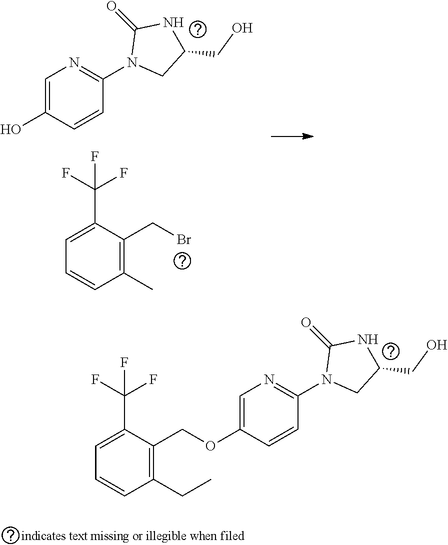

Cesium carbonate (389.37 mg, 1.2 mmol, 2 eq) was added to a suspension of (4S)-4-(hydroxymethyl)-1-(5-hydroxypyridin-2-yl) imidazolidin-2-one (125 mg, 0.6 mmol, 1 eq) and 2-(bromomethyl)-1-ethyl-3-(trifluoromethyl)benzene (175.54 mg, 0.66 mmol, 1.1 eq) in acetonitrile (4 ml) and the suspension stirred for ˜18 hrs. at room temperature to give a pale yellow suspension. Water (25 ml) was added and the mixture extracted with ethyl acetate (60 ml). The extracts were washed with water (2×25 ml) and saturated aqueous sodium chloride solution (25 ml). The solution was dried over anhydrous magnesium sulphate and concentrated to a pale brown gum. The crude product was purified by column chromatography, isolute flash silica (20 g), eluting with dichloromethane/methanol (19:1) to give a yellow green gum. Trituration with diethyl ether (4 ml) gave a white powder, solids were removed by filtration and washed with diethyl ether (10 ml). Dried in vacuo (60° C.). (4S)-1-(5-{[2-Ethyl-6-(trifluoromethyl)phenyl] methoxy}pyridin-2-yl)-4-(hydroxymethyl) imidazolidin-2-one (44 mg, 0.11 mmol, 18.63%).

### Example 322

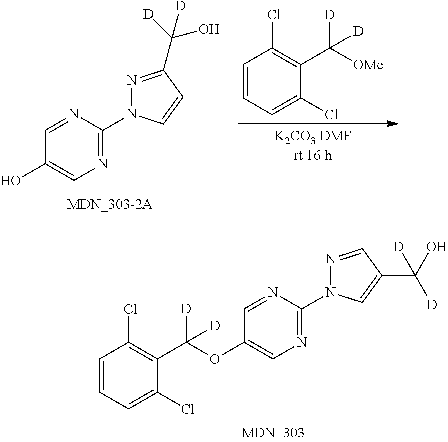 [SMILES: [2H]C([2H])(O)c1ccn(-c2ncc(O)cn2)n1 ; [2H]C([2H])(OC)c1c(Cl)cccc1Cl ; [2H]C([2H])(O)c1cnn(-c2ncc(OC([2H])([2H])c3c(Cl)cccc3Cl)cn2)c1]

A mixture of MDN_303-2A (100 mg, 0.52 mmol), MDN_303-5 (133 mg, 0.52 mmol) and K₂CO₃ (144 mg, 1.04 mmol) in DMSO (1 mL) was stirred at rt overnight. The mixture was quenched with water, extracted with EA and concentrated to give the residue, which was purified by chromatography on silica gel (DCM/MeOH=0-10%) to give the title compound MDN_303 as a white solid (5 mg, 3% yield).

### Example 323

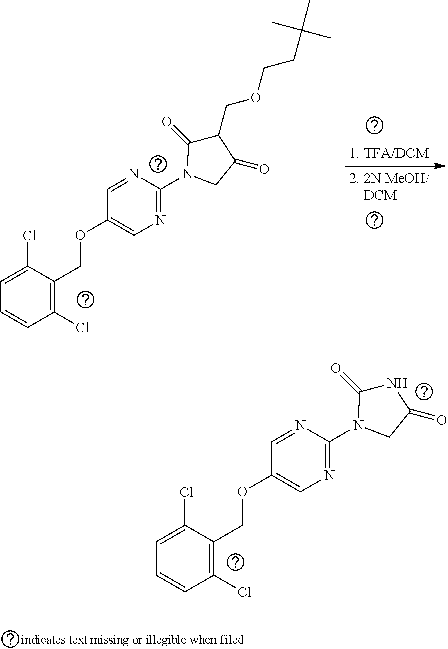 [SMILES: CC(C)(C)CCOCC1C(=O)CN(c2ncc(OCc3c(Cl)cccc3Cl)cn2)C1=O ; O=C1CN(c2ncc(OCc3c(Cl)cccc3Cl)cn2)C(=O)N1]

To a solution of the above starting compound (110 mg, 0.23 mmol, 1 eq) in DCM (2 mL) was added trifluoroacetic acid (87.12 μL, 1.14 mmol, 5 eq) at rt. Stirred for 4 hours The solvent was removed in vacuum and the resultant residue dissolved in THF (2 mL) and 2N aq NaOH (1 mL) added. The reaction was stirred at rt for 1 hour. The reaction mixture was acidified with 2N HCl and diluted with with DCM (5 mL) and water (5 mL). The organic layer was separated, dried over MgSO4 and conc in vacuum. The residue was dissolved into DMSO (2.2 ml) and purified by preparative HPLC automated chromatography (o give: 1-{5-[(2,6-dichlorophenyl) methoxy]pyrimidin-2-yl}imidazolidine-2,4-dione (34 mg, 0.1 mmol, 42.31%) white solid (Purity: >95%).

### Example 324

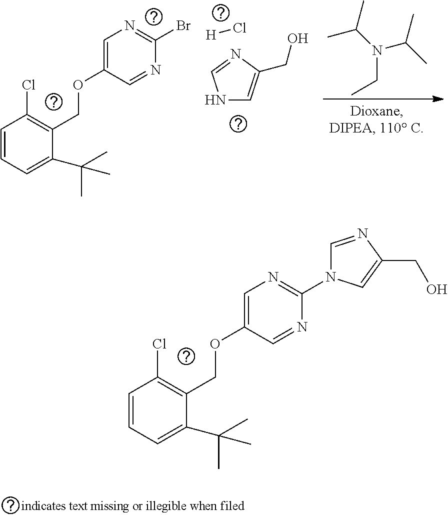

2-bromo-5-{[2-chloro-6-(trifluoromethyl)phenyl] methoxy}pyrimidine (150 mg, 0.41 mmol, 1 eq) and 4-(Hydroxymethyl)-1Himidazole hydrochloride (71.39 mg, 0.53 mmol, 1.3 eq) were taken up in DMSO (2 mL) in a sealed vial. N,N-DIISOPROPYLETHYLAMINE (202.87 μL, 0.78 g/mL, 1.22 mmol, 3 eq) was added. Rxn was sealed and heated to 110oC for 16 h on a timer behind a blast shield. Rxn was filtered. purified by preparative HPLC automated chromatography (ISCO ACCQ HP150, Prep HPLC Column: Big Gemini-NX C18 Dimensions: 30 mm×250 mm 5 μM; Sample: 2.20 ml from tube 1) eluting with 5 to 95% ACN/0.1% FA in Water/0.1% FA F26 to F28 combined and frozen in a CO₂/acetone bath. Rxn was attached to the freeze dried OVERNIGHT. [1-(5-{[2-chloro-6-(trifluoromethyl)phenyl] methoxy}pyrimidin-2-yl) imidazol-4-yl]methanol (1.49 mg, 3.87 μmol, 0.95%)

### Example 326

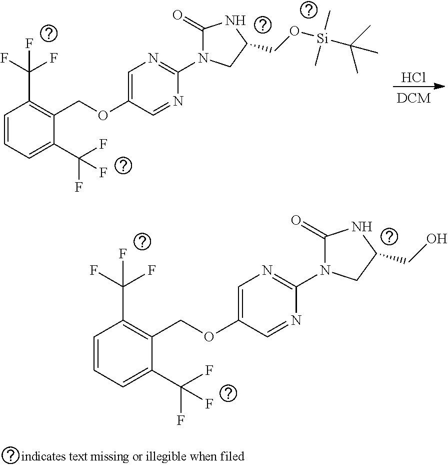 [SMILES: CC(C)(C)[Si](C)(C)OC[C@@H]1CN(c2ncc(OCc3c(C(F)(F)F)cccc3C(F)(F)F)cn2)C(=O)N1 ; Cl ; O=C1N[C@H](CO)CN1c1ncc(OCc2c(C(F)(F)F)cccc2C(F)(F)F)cn1]

HYDROCHLORIC ACID (4N in 1,4-dioxane) (0.05 mL, 4M in, 0.21 mmol, 5 eq) was added to a stirred solution of (4S)-1-(5-{[2,6-bis (trifluoromethyl)phenyl] methoxy}pyrimidin-2-yl)-4-{[(tert-butyldimethylsilyl)oxy] methyl}imidazolidin-2-one (23 mg, 0.04 mmol, 1 eq) in DCM (2 mL). The reaction was stirred at rt for ca 1 h. The solvent was removed in vacuo then the resultant residue dissolved in DMSO, filtered and purified by preparative HPLC automated chromatography (ISCO ACCQ HP150, Prep HPLC Column: Gemini-NX C18 Dimensions: 21 mm×150 mm 5 μM; Sample: 1.30 ml from tube 1) eluting with 5 to 95% ACN/0.1% FA in Water/0.1% FA. Fraction 4 was freeze dried giving (4S)-1-(5-{[2,6-bis (trifluoromethyl)phenyl] methoxy}pyrimidin-2-yl)-4-(hydroxymethyl) imidazolidin-2-one (3.02 mg, 0.01 mmol, 16.57%) as a white solid (Purity: >95%).

### Example 327

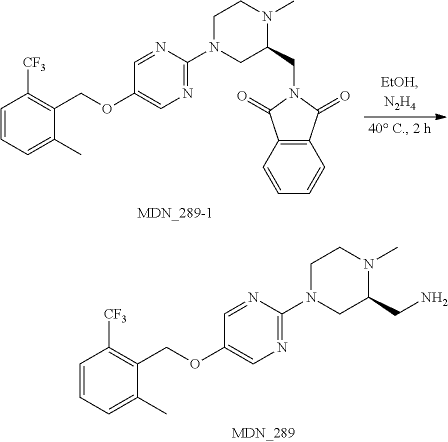 [SMILES: Cc1cccc(C(F)(F)F)c1COc1cnc(N2CCN(C)[C@@H](CN3C(=O)c4ccccc4C3=O)C2)nc1 ; Cc1cccc(C(F)(F)F)c1COc1cnc(N2CCN(C)[C@@H](CN)C2)nc1]

To a mixture of MDN_289-1 (150 mg, 0.270 mmol) in EtOH (4 mL) was added hydrazine hydrate (2 mL) at 0oC. The reaction was stirred at 40oC for 2 h. The reaction mixture was diluted with H2O (30 mL) and extracted with EtOAc (30 mL×3). The organic layer was dried over Na2SO4, filtered and concentrated in vacuo. The crude product was purified by prep-HPLC (NH4HCO3) to afford MDN_289 (11 mg, 13.0% yield).

### Example 328

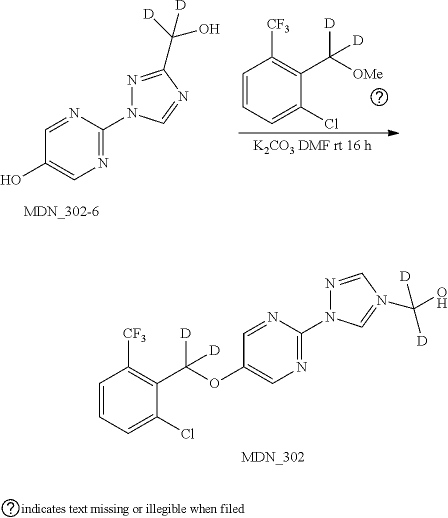

A mixture of MDN_302-6 (100 mg, 0.51 mmol), MDN_302-5 (149 mg, 0.51 mmol) and K₂CO₃ (138 mg, 1.0 mmol) in DMSO (1 mL) was stirred at rt overnight. The mixture was quenched with water, extracted with EA and concentrated to give the residue, which was purified by chromatography on silica gel (DCM/MeOH=0-10%) to give the title compound MDN_302 as a white solid (3.16 mg, 3% yield).

### Example 329

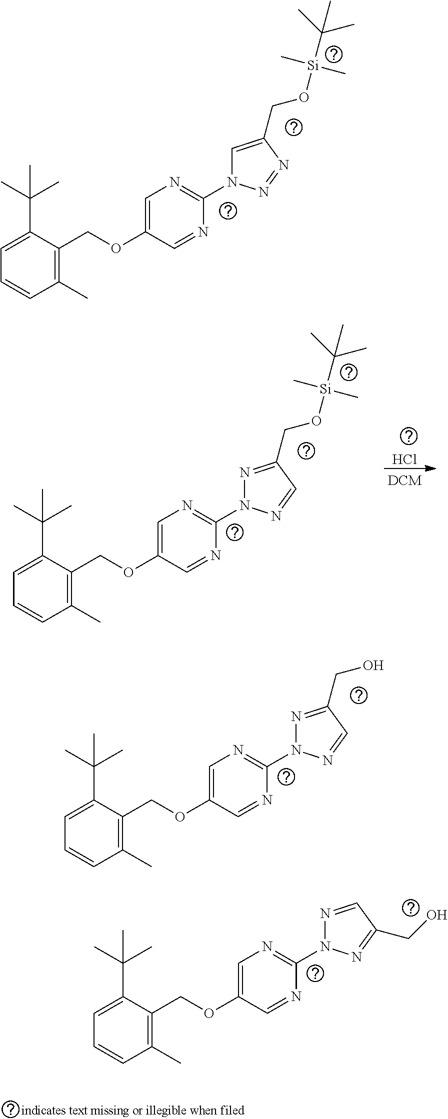 [SMILES: Cc1cccc(C(C)(C)C)c1COc1cnc(-n2cc(CO[Si](C)(C)C(C)(C)C)nn2)nc1 ; Cc1cccc(C(C)(C)C)c1COc1cnc(-n2ncc(CO[Si](C)(C)C(C)(C)C)n2)nc1 ; Cl ; Cc1cccc(C(C)(C)C)c1COc1cnc(-n2ncc(CO)n2)nc1 ; Cc1cccc(C(C)(C)C)c1COc1cnc(-n2ncc(CO)n2)nc1]

HYDROCHLORIC ACID (4N in 1,4-dioxane) (0.19 mL, 4M in, 0.77 mmol, 5 eq) was added to a stirred solution of 2-(4-{[(tertbutyldimethylsilyl)oxy] methyl}-1,2,3-triazol-1-yl)-5-{[2-methyl-6-(trifluoromethyl)phenyl] methoxy}pyrimidine and 2-(4-{[(tert-butyldimethylsilyl)oxy] methyl}-1,2,3-triazol-2-yl)-5-{[2-methyl-6-(trifluoromethyl)phenyl] methoxy}pyrimidine (74 mg, 0.15 mmol, 1 eq) in DCM (2 mL). The reaction was stirred at rt for ca 2.5 h. The resultant residue was dissolved in DMSO then purified by preparative HPLC automated chromatography (ISCO ACCQ HP150, Prep HPLC Column: Big Gemini-NX C18 Dimensions: 30 mm×250 mm 5 μM; Sample: 1.50 ml from tube 1) eluting with 5 to 95% ACN/0.1% FA in Water/0.1% FA Dimensions: 30 mm×250 mm 5 μM; Sample: 1.50 ml from tube 1) eluting with 5 to 95% ACN/0.1% FA in Water/0.1% FA Fractions giving [2-(5-{[2-methyl-6-(trifluoromethyl)phenyl] methoxy}pyrimidin-2-yl)-1,2,3-triazol-4-yl] methanol as a white solid (Purity: >95%) and [1-(5-{[2-methyl-6-(trifluoromethyl)phenyl] methoxy}pyrimidin-2-yl)-1,2,3-triazol-4-yl]methanol mmol) as a white solid (Purity: >95%).

### Example 330

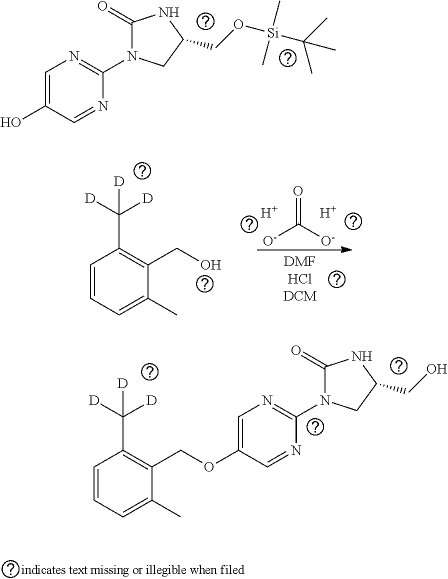

(4S)-4-{[(tert-butyldimethylsilyl)oxy] methyl}-1-(5-hydroxypyrimidin-2-yl) imidazolidin-2-one (33 mg, 0.1 mmol, 1 eq), 2-(Bromomethyl)-1-methyl-3-(trifluoromethyl)benzene (25.74 mg, 0.1 mmol, 1 eq) and POTASSIUM CARBONATE (21.09 mg, 0.15 mmol, 1.5 eq) in DMF (2 mL) was stirred at rt overnight. The reaction was diluted with EtOAc then washed with water followed by brine. The organic extract was dried (MgSO4) then the solvent removed in vacuo. The resultant residue was dissolved in DCM (2 mL) then HYDROCHLORIC ACID (4N in 1,4-dioxane) (0.1 mL, 4M in, 0.41 mmol, 4 eq) added. The reaction was stirred at rt for ca 1.5 h. The solvent was removed in vacuo then the resultant residue dissolved in DMSO, filtered and purified by preparative HPLC automated chromatography (ISCO ACCQ HP150, Prep HPLC Column: Gemini-NX C18 Dimensions: 21 mm×150 mm 5 μM; Sample: 1.30 ml from tube 1) eluting with 5 to 95% ACN/0.1% FA in Water/0.1% FA. Fraction 4 was freeze dried giving (4S)-4-(hydroxymethyl)-1-(5-{[2-methyl-6-(trifluoromethyl)phenyl] methoxy}pyrimidin-2-yl) imidazolidin-2-one (10.87 mg, 0.03 mmol, 27.95%) as a white solid (Purity: >95%).

### Example 331

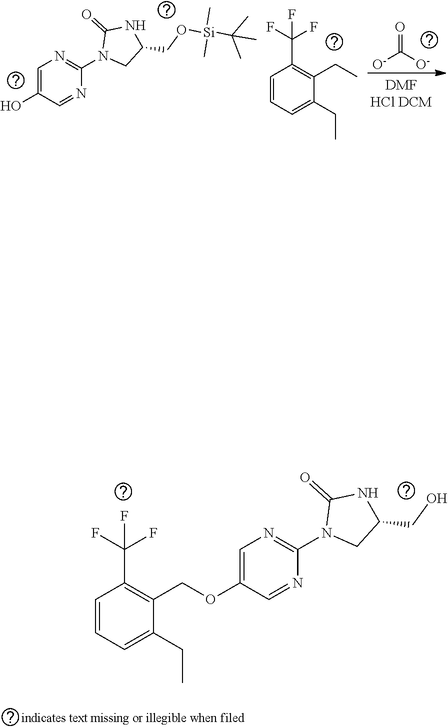

(4S)-4-{[(tert-butyldimethylsilyl)oxy] methyl}-1-(5-hydroxypyrimidin-2-yl) imidazolidin-2-one (33 mg, 0.1 mmol, 1 eq), 2-(bromomethyl)-1-ethyl-3-(trifluoromethyl)benzene (27.17 mg, 0.1 mmol, 1 eq) and POTASSIUM CARBONATE (21.09 mg, 0.15 mmol, 1.5 eq) in DMF (2 mL) was stirred at rt overnight. LCMS shows the reaction has gone to completion with some impurities present. The reaction was diluted with EtOAc then washed with water followed by brine. The organic extract was dried (MgSO4) then the solvent removed in vacuo. The resultant residue was dissolved in DCM (2 mL) then HYDROCHLORIC ACID (4N in 1,4-dioxane) (0.1 mL, 4M in, 0.41 mmol, 4 eq) added. The reaction was stirred at rt for ca 1.5 h. LCMS The solvent was removed in vacuo then the resultant residue dissolved in DMSO, filtered and purified by preparative HPLC automated chromatography (ISCO ACCQ HP150, Prep HPLC Column: Gemini-NX C18 Dimensions: 21 mm×150 mm 5 μM; Sample: 1.30 ml from tube 1) eluting with 5 to 95% ACN/0.1% FA in Water/0.1% FA. Fraction 3 was freeze dried giving (4S)-1-(5-{[2-ethyl-6-(trifluoromethyl)phenyl] methoxy}pyrimidin-2-yl)-4-(hydroxymethyl) imidazolidin-2-one (10.01 mg, 0.03 mmol, 24.83%) as a white solid (Purity: >95%).

### Example 334

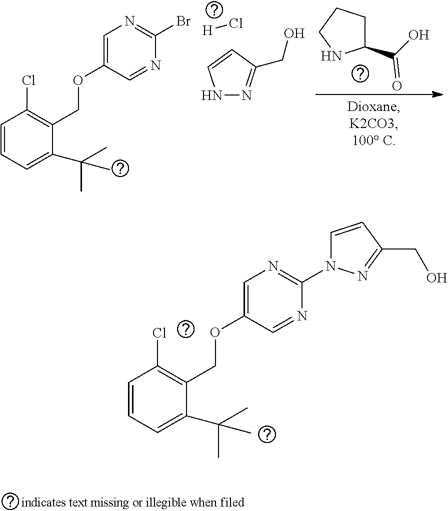

2-bromo-5-{[2-chloro-6-(trifluoromethyl)phenyl] methoxy}pyrimidine (161 mg, 0.44 mmol, 1 eq) was taken up in DMSO (3 mL) in a sealed tube. 2H-pyrazol-3-ylmethanol HCl (70.73 mg, 0.53 mmol, 1.2 eq) L-PROLINE (0.01 mL, 1.35 g/mL, 0.09 mmol, 0.2 eq) and potassium carbonate (181.62 mg, 1.31 mmol, 3 eq) were added. Rxn was sparged with N2. copper (I) iodide (8.34 mg, 0.04 mmol, 0.1 eq) was added. Rxn was further sparged and heated to 110oC behind a blast shield overnight. Rxn was partitioned between EtOAc/H2O, followed by brine. Organics were dried over MgSO4, filtered and conc in vacuo. Then taken up in 2 ml DMSO and filtered. purified by preparative HPLC automated chromatography to give [1-(5-{[2-chloro-6-(trifluoromethyl)phenyl] methoxy}pyrimidin-2-yl) pyrazol-3-yl]methanol (9.22 mg, 0.02 mmol, 5.47%) as a white solid.

### Example 335

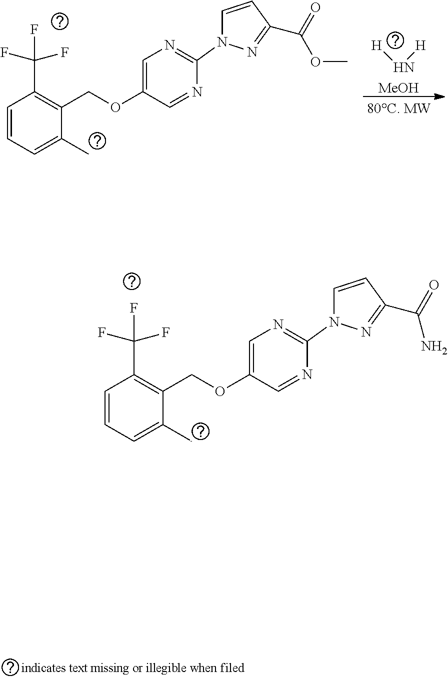

methyl 1-(5-{[2-methyl-6-(trifluoromethyl)phenyl] methoxy}pyrimidin-2-yl) pyrazole-3-carboxylate, 3564-SB-120-001 (147 mg, 0.37 mmol, 1 eq) was taken up in ammonia (5.03 mL, 7M in, 35.22 mmol, 94 eq) (in MeOH) in a screw top tube which was sealed and heated to 80oC behind a blast shield for 3 h on a timer. AM, rxn ⅔ complete by LC-MS. heating was continued for a further 90 min. Rxn now 80% complete by LC-MS. Rxn was conc in vacuo, retaken up in DCM and loaded onto isolute. purified by automated flash chromatography (Combiflash 300+, Silica 4 g RediSep column) eluting with 0 to 5% Methanol in Dichloromethane purified by automated flash chromatography (Combiflash 300+, Silica 4 g RediSep column) eluting with 0 to 5% Methanol in Dichloromethane F5 to F7 combined and conc in vacuo. The residue was taken up in minimal DCM, then blown down into a submission vial and dried at 40oC under vacuum OVERNIGHT. 1-(5-{[2-methyl-6-(trifluoromethyl)phenyl] methoxy}pyrimidin-2-yl) pyrazole-3-carboxamide (75.73 mg, 0.2 mmol, 53.57%) isolated as a white solid.

### Example 337

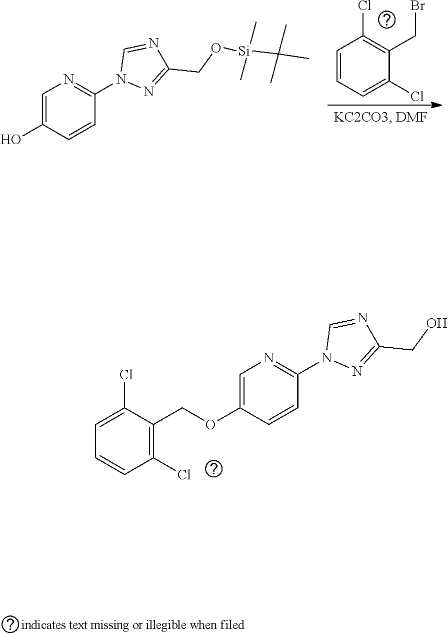

To a solution of the above starting compound (50 mg, 0.16 mmol, 1 eq) in DMF (2 mL) was added 2,6-Dichlorobenzyl bromide (46.98 mg, 0.2 mmol, 1.2 eq) and potassium carbonate (45.1 mg, 0.33 mmol, 2 eq) stirred at RT overnight. The reaction mixture was diluted with EtOAc (5 mL), filtered and conc in vacuum HCl (4M in Dioxane) (2 mL) was added and the reaction mixture stirred at rt for 3 hours. The reaction mixture was conc in vacuum, DMSO (2 mL) was added and purified by preparative HPLC automated chromatography (ISCO ACCQ HP150, Prep HPLC Column: Gemini-NX C18 Dimensions: 21 mm×150 mm 5 μM; Sample: 2.00 ml from tube 1) eluting with 5 to 95% ACN/0.1% FA in Water/0.1% FA fractions 7-9 to give: (1-{5-[(2,6-dichlorophenyl) methoxy]pyridin-2-yl}-1,2,4-triazol-3-yl) methanol (40 mg, 0.11 mmol, 69.81%) as a white solid (Purity: >98%).

### Example 339

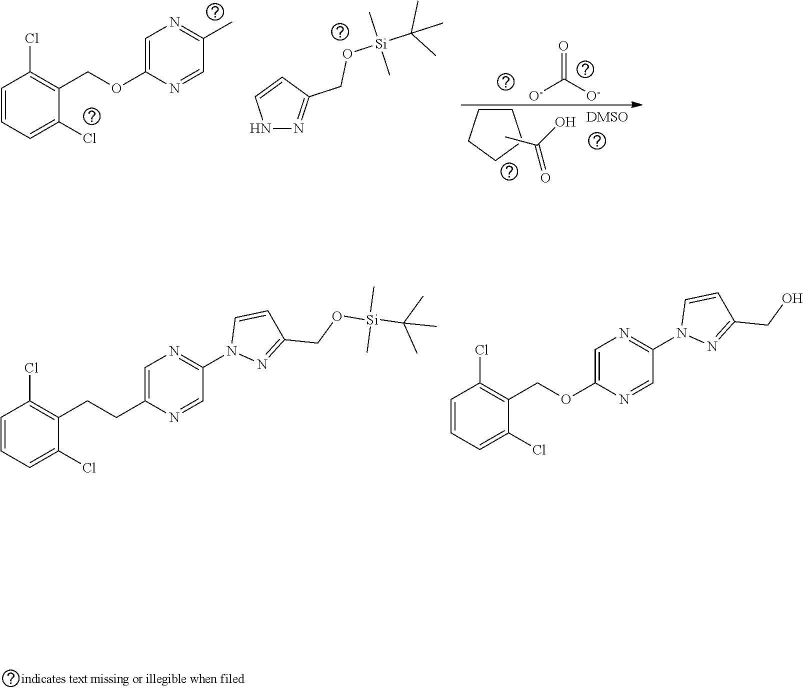

Nitrogen bubbled through a solution of 2-bromo-5-[(2,6-dichlorophenyl) methoxy]pyrazine (200 mg, 0.6 mmol, 1 eq), 3-{[(tert-butyldimethylsilyl)oxy] methyl}-1H-pyrazole (152.6 mg, 0.72 mmol, 1.2 eq), POTASSIUM CARBONATE (248.28 mg, 1.8 mmol, 3 eq) and L-PROLINE (13.79 mg, 0.12 mmol, 0.2 eq) in DMSO (4 mL) then COPPER (I) IODIDE (11.4 mg, 0.06 mmol, 0.1 eq) added. The reaction was sealed then heated to 90oC, where it was maintained overnight. The reaction was cooled to rt then diluted with EtOAc and washed with water followed by brine. The organic extract was dried (MgSO4) then the solvent removed in vacuo. The resultant residue was dissolved in DCM (3 mL) then HCl (4N in 1,4-dioxane, 0.3 mL) was added. The reaction was stirred at rt for ca 2 h. The solvent was removed in vacuo then the resultant residue dissolved in DMSO, filtered and purified by preparative HPLC automated chromatography (ISCO ACCQ HP150, Prep HPLC Column: Gemini-NX C18 Dimensions: 21 mm×150 mm 5 μM; Sample: 1.40 ml from tube 1) eluting with 5 to 95% ACN/0.1% FA in Water/0.1% FA. eluting with 5 to 95% ACN/0.1% FA in Water/0.1% FA. Fraction 28 was freeze dried giving (1-{5-[(2,6-dichlorophenyl) methoxy]pyrazin-2-yl}pyrazol-3-yl) methanol (8.51 mg, 0.02 mmol, 4.05%) as a beige solid (Purity: >95%).

### Example 340

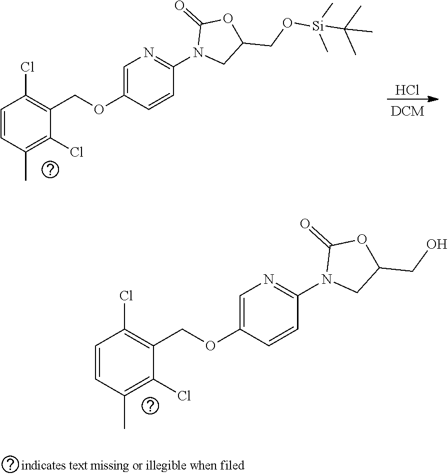 [SMILES: Cc1ccc(Cl)c(COc2ccc(N3CC(CO[Si](C)(C)C(C)(C)C)OC3=O)nc2)c1Cl ; Cl ; Cc1ccc(Cl)c(COc2ccc(N3CC(CO)OC3=O)nc2)c1Cl]

HYDROCHLORIC ACID (4N in 1,4-dioxane), VERC100374 (0.1 mL, 4M in, 0.39 mmol, 4 eq) was added to a stirred solution of 5-{[(tertbutyldimethylsilyl)oxy] methyl}-3-{5-[(2,6-dichloro-3-fluorophenyl) methoxy]pyridin-2-yl}-1,3-oxazolidin-2-one (48.5 mg, 0.1 mmol, 1 eq) in DCM (2 mL). The reaction was stirred at rt over the weekend. LCMS shows the reaction has gone to completion. The solvent was removed in vacuo then the resultant residue dissolved in in DMSO and purified by preparative HPLC automated chromatography (ISCO ACCQ HP150, Prep HPLC Column: Gemini-NX C18 Dimensions: 21 mm×150 mm 5 μM; Sample: 1.30 ml from tube 1) eluting with 5 to 95% ACN/0.1% FA in Water/0.1% FA. Fractions 3 & 4 were combined then freeze dried giving 3-{5-[(2,6-dichloro-3-fluorophenyl) methoxy]pyridin-2-yl}-5-(hydroxymethyl)-1,3-oxazolidin-2-one (30.41 mg, 0.08 mmol, 81.2%) as a white solid (Purity: >95%).

### Example 346

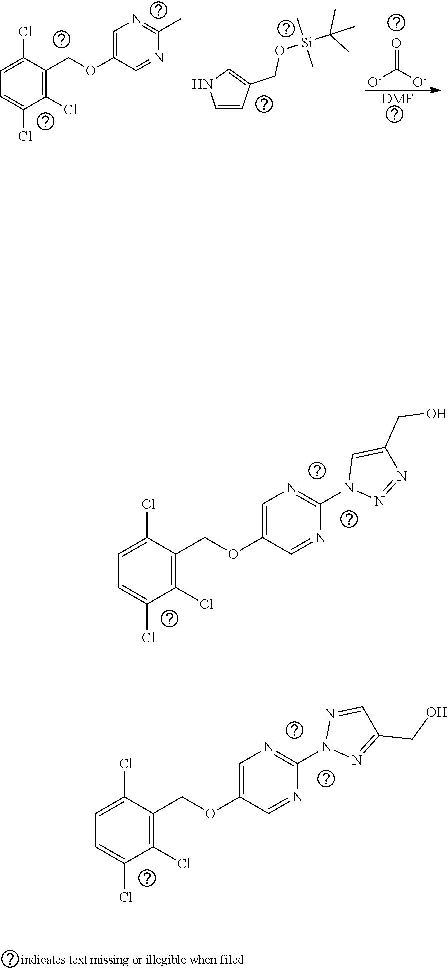

Nitrogen was bubbled through a solution of 2-bromo-5-[(2,6-dichloro-3-fluorophenyl) methoxy]pyrimidine (323 mg, 0.92 mmol, 1 eq), 4-{[(tert-butyldimethylsilyl)oxy] methyl}-1H-1,2,3-triazole (195.78 mg, 0.92 mmol, 1 eq) and POTASSIUM CARBONATE (253.65 mg, 1.84 mmol, 2 eq) in DMF (5 mL). The tube was sealed then heated to 80oC, where it was maintained overnight. The reaction was cooled to rt then diluted with EtOAc and washed with water followed by brine. The organic extract was dried (MgSO4) then the solvent removed in vacuo. To remove any remaining TBDMS protecting group the resultant residue was dissolved in DCM (3 mL) then HYDROCHLORIC ACID (4N in 1,4-dioxane), VERC100374 (0.3 mL, 4M in, 1.2 mmol, 1.31 eq) added. The reaction was stirred at rt for ca 2 h. The solvent was removed in vacuo then the resultant residue dissolved in DMSO, filtered and purified by preparative HPLC automated chromatography (ISCO ACCQ HP150, Prep HPLC Column: Big Gemini-NX C_{1 8} Dimensions: 30 mm×250 mm 5 μM; Sample: 1.80 ml from tube 1) eluting with 5 to 95% ACN/0.1% FA in Water/0.1% FA Fractions 48 & 49 (3584-RJP-071-001) were combined then freeze dried giving (2-{5-[(2,6-dichloro-3-fluorophenyl) methoxy]pyrimidin-2-yl}-1,2,3-triazol-4-yl) methanol (33.73 mg, 0.09 mmol, 9.93%) as a white solid (Purity: 90-95%) Fractions 51 & 52 were combined then freeze dried giving (2-{5-[(2,6-dichloro-3-fluorophenyl) methoxy]pyrimidin-2-yl}-1,2,3-triazol-4-yl) methanol (33.73 mg, 0.09 mmol, 9.93%) as a white solid (Purity: >95%).

### Example 356

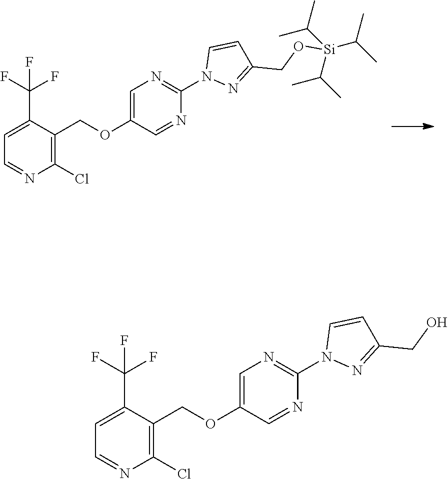 [SMILES: CC(C)[Si](OCc1ccn(-c2ncc(OCc3c(C(F)(F)F)ccnc3Cl)cn2)n1)(C(C)C)C(C)C ; OCc1ccn(-c2ncc(OCc3c(C(F)(F)F)ccnc3Cl)cn2)n1]

Tetrabutylammonium fluoride solution (1M in THF) (1 ml (1M), 1.02 mmol, 2 eq) was added to a solution of 5-{[2-chloro-4-(trifluoromethyl)pyridin-3-yl]methoxy}-2-(3-{[(triisopropylsilyl)oxy] methyl}pyrazol-1-yl)pyrimidine (425 mg (65%), 0.51 mmol, 1 eq) in THF (5 ml) at 0oC (ice/water) under a nitrogen atmosphere and the mixture stirred. The solution was stirred for ˜60 mins. at 0° C. to give a pale brown solution and water (25 ml) was added. The mixture was extracted with ethyl acetate (50 ml) and the extracts were washed with water (3×25 ml) and saturated aqueous sodium chloride solution (25 ml). The solution was dried over anhydrous magnesium sulphate and concentrated to a brown oil. The crude product was purified by column chromatography, isolute flash silica (20 g), eluting with dichloromethane/methanol (39:1) to give a pale brown foam. Dried in vacuo. 2 The crude product was repurified by column chromatography, isolute flash silica (10 g), eluting with ethyl acetate to give a colourless gum. Dichloromethane (2 ml) added, transferred to a sample vial and concentrated in vacuo. Solidified on standing.to give [1-(5-{[2-Chloro-4-(trifluoromethyl)pyridin-3-yl]methoxy}pyrimidin-2-yl) pyrazol-3-yl]methanol.

### Example 362

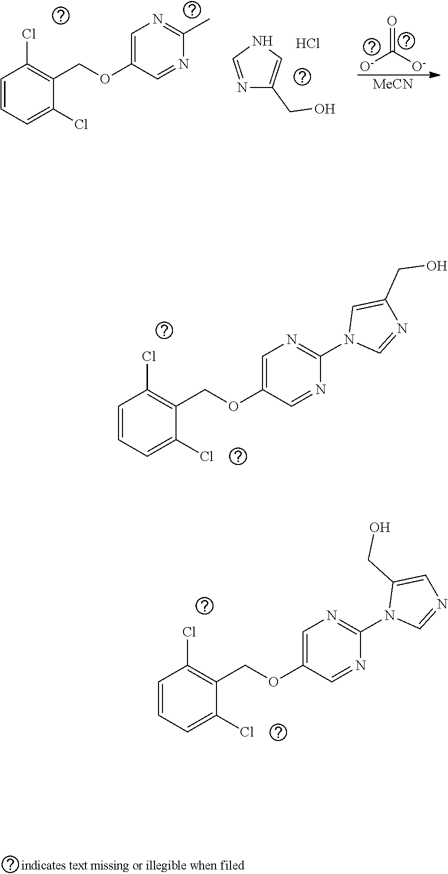

2-bromo-5-[(2,6-dichlorophenyl) methoxy]pyrimidine (1 g, 2.99 mmol, 1 eq), 4-(Hydroxymethyl)-1H-imidazole hydrochloride (483.47 mg, 3.59 mmol, 1.2 eq) and CAESIUM CARBONATE (1.95 g, 5.99 mmol, 2 eq) in Acetonitrile (30 mL) was heated to 75oC, where it was maintained overnight. LCMS shows significant starting material still present. The reaction was maintained at 75oC for a further ca 5 h. LCMS shows no major change. Further 4-(Hydroxymethyl)-1H-imidazole hydrochloride (120.87 mg, 0.9 mmol, 0.3 eq) was added then stirring maintained at 75oC for a further ca 2.5 h (no major improvement by LCMS) then at rt overnight.

The reaction was diluted with EtOAc then washed with water followed by brine. The organic extract was dried (MgSO4) then absorbed on to isolute and purified by automated flash chromatography (Combiflash 300+, Silica 40 g Gold RediSep column) eluting with 0 to 100% Ethyl Acetate in Heptane. Fractions 51-75 (rack 1)+1-3 (rack 2) were combined then the solvent removed in vacuo. The resultant residue was triturated in diethyl ether then collected by filtration giving a white solid (122 mg) NMR shows compound is not pure enough for submission.

A gum was still present on the flask used to absorb the compound on to isolute prior to being columned. LCMS confirms compound is present. The flask was washed with MeOH then filtered and the filtrate absorbed on to isolute. Purified by automated flash chromatography (Combiflash 300+, Silica 12 g Gold RediSep column) eluting with 0 to 100% Ethyl Acetate in Heptane. Fractions 21-31 were combined then the solvent removed in vacuo.

The resultant residue was triturated in diethyl ether then collected by filtration. Gave a further 12 mg. 300+, Silica 12 g Gold RediSep column) eluting with 0 to 100% Ethyl Acetate in Heptane. Fractions 21-31 were combined then the solvent removed in vacuo. The resultant residue was triturated in diethyl ether then collected by filtration. Gave a further 12 mg. The solids were combined then attempted to dissolve in DMSO for further purification by prep. Unfortunately the compound is too insoluble. The DMSO was removed on the Biotage V10 then the resultant residue dissolved in DCM/MeOH and absorbed on to isolute. Further purified by automated flash chromatography (Combiflash 300+, Silica 24 g Gold RediSep column) eluting with 0 to 10% Methanol in Dichloromethane. Fractions 23-26 were combined then the solvent removed in vacuo giving (1-{5-[(2,6-dichlorophenyl) methoxy]pyrimidin-2-yl}imidazol-4-yl) methanol (91.69 mg, 0.26 mmol, 8.72%) as a white solid (Purity: >98%). Fractions 32-47 were also collected from the first column. The solvent was removed in vacuo then the resultant residue triturated in diethyl ether and collected by filtration giving (3-{5-[(2,6-dichlorophenyl) methoxy]pyrimidin-2-yl}imidazol-4-yl) methanol (57.58 mg, 0.16 mmol, 5.48%) as a white solid (Purity: >95%).

### Example 363

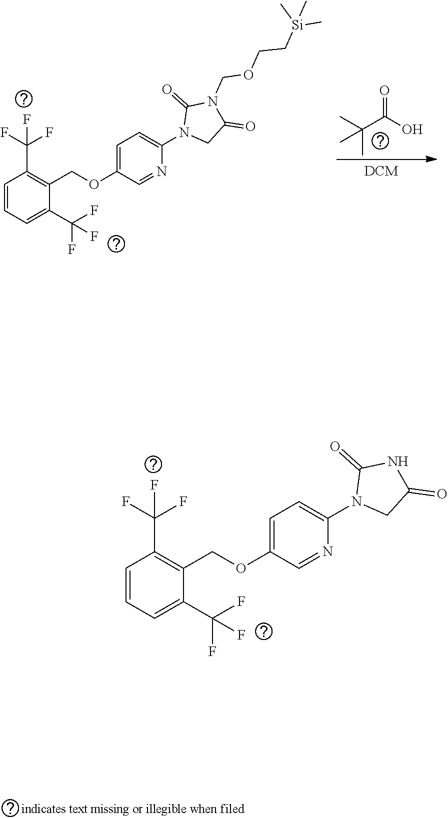

TRIFLUOROACETIC ACID (0.05 mL, 1.53 g/mL, 0.68 mmol, 4 eq) was added to a stirred solution of 1-(5-{[2,6-bis (trifluoromethyl)phenyl] methoxy}pyridin-2-yl)-3-{[2-(trimethylsilyl) ethoxy]methyl}imidazolidine-2,4-dione (93.6 mg, 0.17 mmol, 1 eq) in DCM (5 mL). The reaction was stirred at rt overnight. LCMS shows mainly intermediate (CH₂OH from the SEM still present) with some starting material present. Further TRIFLUOROACETIC ACID (0.05 mL, 1.53 g/mL, 0.68 mmol, 4 eq) was added and stirring contined at rt for ca 24 h. LCMS shows the reaction has gone completely to the intermediate (CH₂OH from the SEM still present) The solvent was removed in vacuo then the resultant residue dissolved in THF (2 mL) and 2N aq NaOH (0.2 mL) added. The reaction was stirred at rt for ca 2 h. LCMS shows the reaction has gone to completion. The solvent was removed in vacuo then the resultant residue dissolved in DMSO, filtered and purified by preparative HPLC automated chromatography (ISCO ACCQ HP150, Prep HPLC Column: Gemini-NX C18 Dimensions: 21 mm×150 mm 5 μM; Sample: 1.30 ml from tube 1) eluting with 5 to 95% ACN/0.1% FA in Water/0.1% FA. Fractions 4 & 5 were combined then freeze dried. Gave a mixture of product and intermediate as a white solid. The resultant solid was dissolved in THF (2 mL) then 2N aq NaOH (0.5 mL) added. The reaction was stirred at rt for ca 5 h. LCMS suggests the reaction has gone to completion. The reaction was partioned between DCM and water then the layers separated. The aq was further extracted with 3:1 DCM/IPA then the organic extracts combined, washed with brine, dried (MgSO4) and the solvent removed in vacuo. The resultant residue was transferred to a submission vial using DCM/MeOH then the solvent removed under a stream of nitrogen and dried overnight under vacuum at 45oC. Gave 1-(5-{[2,6-bis (trifluoromethyl)phenyl] methoxy}pyridin-2-yl) imidazolidine-2,4-dione (31.39 mg, 0.07 mmol, 43.96%) as a white solid (Purity: 90-95%).

### Example 364

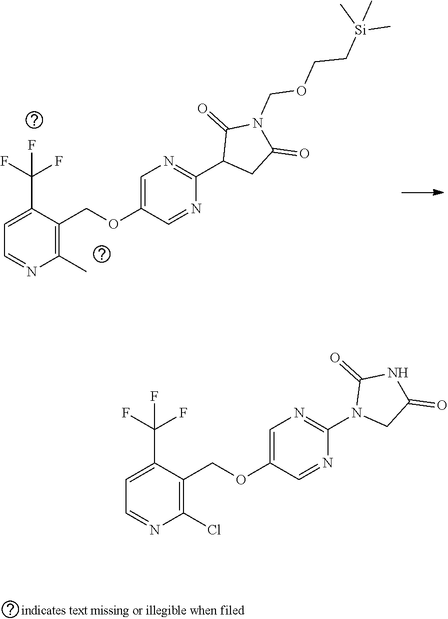 [SMILES: Cc1nccc(C(F)(F)F)c1COc1cnc(C2CC(=O)N(COCC[Si](C)(C)C)C2=O)nc1 ; O=C1CN(c2ncc(OCc3c(C(F)(F)F)ccnc3Cl)cn2)C(=O)N1]

Hydrogen chloride (4M in 1,4-dioxane) (3 ml) was added to a solution of 1-(5-{[2-chloro-4-(trifluoromethyl)pyridin-3-yl]methoxy}pyrimidin-2-yl)-3-{[2-(trimethylsilyl) ethoxy]methyl}imidazolidine-2,4-dione (135 mg (90%), 0.23 mmol, 1 eq) in 1,4-dioxane (1 ml) at 0° C. (ice/water) and the resulting suspension stirred. The suspension was stirred at room temperature for ˜7 days to give a white suspension. The suspension was concentrated and THF (4 ml) added to give a pale brown suspension, aqueous sodium hydroxide (2 ml, 2M) was added and the mixture stirred for ˜2 hrs. Saturated aqueous ammonium chloride solution (25 ml) was added and the mixture was extracted with ethyl acetate (50 ml). The extracts were washed with water (2×25 ml) and saturated aqueous sodium chloride solution (25 ml). The solution was dried over anhydrous magnesium sulphate and concentrated to a white solid. The crude product was purified by column chromatography, isolute flash silica (10 g), eluting with dichloromethane/methanol (39:1) to give a white solid. Trituration with diethyl ether (2 ml) gave a white powder, solids were removed by filtration and washed with diethyl ether (10 ml). Dried in vacuo (60oC). Rf 0.15 CH2C12/MeOH (19:1) SiO2 1-(5-{[2-Chloro-4-(trifluoromethyl)pyridin-3-yl]methoxy}pyrimidin-2-yl) imidazolidine-2,4-dione (44 mg, 0.11 mmol, 48.38%)

### Example 366

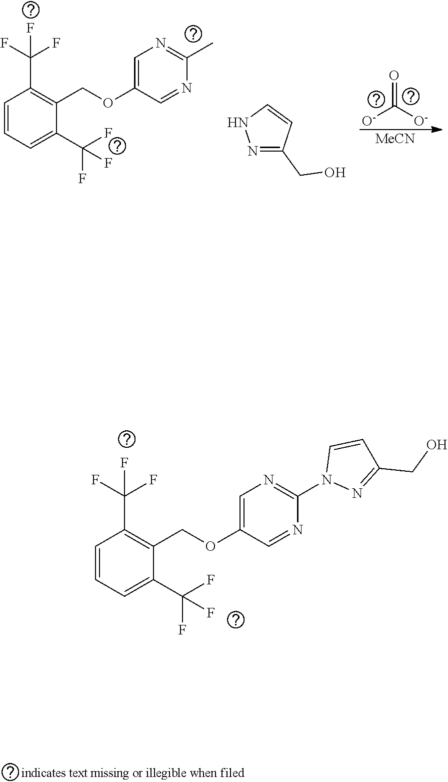

A solution of 5-{[2,6-bis (trifluoromethyl)phenyl] methoxy}-2-bromopyrimidine (300 mg, 0.75 mmol, 1 eq), 1H-Pyrazole-3-methanol 97+% (88.05 mg, 0.9 mmol, 1.2 eq) and CAESIUM CARBONATE (487.39 mg, 1.5 mmol, 2 eq) in Acetonitrile (8 mL) was heated to 75° C., where it was maintained overnight. LCMS suggests product present. The reaction was cooled to rt then diluted with EtOAc and washed with water followed by brine. The organic extract was dried (MgSO4) then the solvent removed in vacuo. The resultant residue was dissolved in DMSO, filtered then purified by preparative HPLC automated chromatography (ISCO ACCQ HP150, Prep HPLC Column: Big Gemini-NX C18 Dimensions: 30 mm×250 mm 5 μM; Sample: 1.60 ml from tube 1) eluting with 5 to 95% ACN/0.1% FA in Water/0.1% FA. Fractions 40 & 41 were combined then freeze-dried giving [1-(5-{[2,6-bis (trifluoromethyl)phenyl] methoxy}pyrimidin-2-yl) pyrazol-3-yl]methanol (59.56 mg, 0.14 mmol, 19.04%) as a white solid (Purity: 85-90%). The compound was dissolved in minimum DCM then further purified by automated flash chromatography (Combiflash 300+, Silica 12 g Gold RediSep column) eluting with 0 to 10% Methanol in Dichloromethane. Fractions 2 & 3 were combined then the solvent removed in vacuo giving [1-(5-{[2,6-bis (trifluoromethyl)phenyl] methoxy}pyrimidin-2-yl) pyrazol-3-yl]methanol (51.22 mg, 0.12 mmol, 16.37%) as a colourless glass (Purity: >95%).

### Example 368

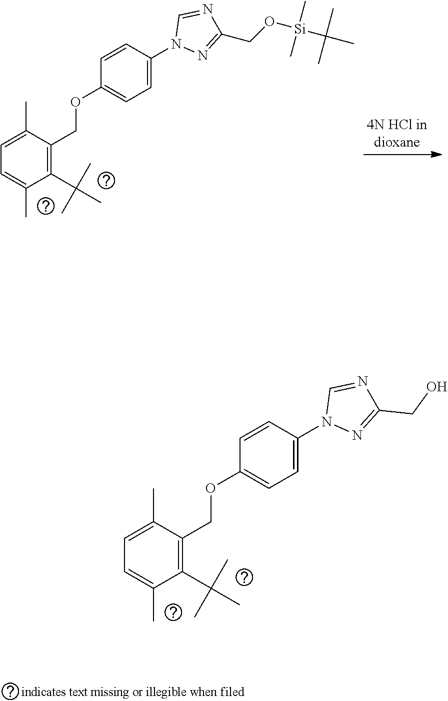 [SMILES: Cc1ccc(C)c(C(C)(C)C)c1COc1ccc(-n2cnc(CO[Si](C)(C)C(C)(C)C)n2)cc1 ; Cc1ccc(C)c(C(C)(C)C)c1COc1ccc(-n2cnc(CO)n2)cc1]

A solution of the silyl-protected compound above (200 mg, 0.4 mmol, 1 eq) in HCl (4M in Dioxane) (2 mL) was stirred at room temperature for 3 hours. The reaction mixture was concentrated in vacuum, dissolved into DMSO (2×2.8 mL) and purified by preparative HPLC automated chromatography (ISCO ACCQ HP125, Prep HPLC Column: Big Gemini-NX C18 Dimensions: 30 mm×250 mm 5 μM; Sample: 2.80 ml from tube 1) eluting with 5 to 95% ACN/0.1% FA in Water/0.1% FA fractions 5-6 and 15-16 to give: [1-(4-{[3-fluoro-6-methyl-2-(trifluoromethyl)phenyl] methoxy}phenyl)-1,2,4-triazol-3-yl]methanol (71 mg, 0.19 mmol, 46.14%) white solid (Purity: >98%)

### Example 369

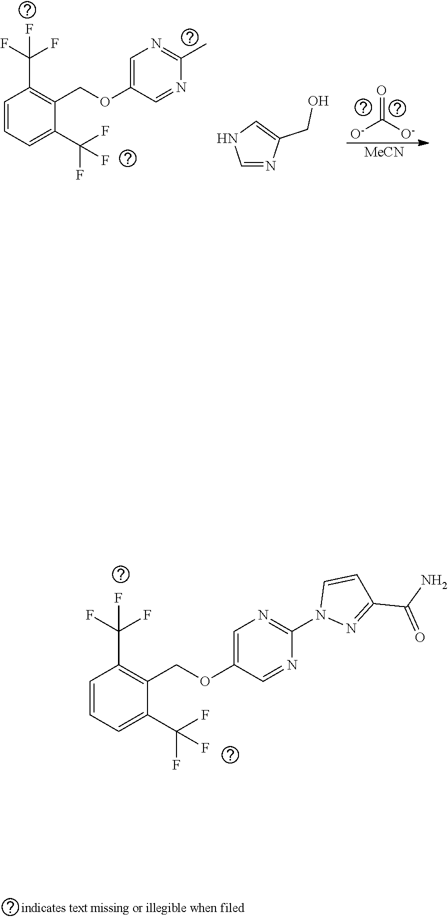

5-{[2,6-bis (trifluoromethyl)phenyl] methoxy}-2-bromopyrimidine (600 mg, 1.5 mmol, 1 eq), 1H-Imidazol-4-ylmethanol (176.1 mg, 1.8 mmol, 1.2 eq) and CAESIUM CARBONATE (974.77 mg, 2.99 mmol, 2 eq) in Acetonitrile (15 mL) was heated to 75° C., where it was maintained overnight. LC-MS showed mainly product plus some starting material and some double addition of the imidazole. The reaction was cooled to room temperature then diluted with EtOAc and washed with water followed by brine. The organic extract was dried (MgSO4) then the solvent removed in vacuo. The resultant residue was purified by automated flash chromatography (Combiflash 300+, Silica 24 g RediSep column) eluting with 0 to 20% Methanol in Dichloromethane. Fractions 14-18 were combined then the solvent removed in vacuo then the resultant residue triturated in diethyl ether and collected by filtration to give [1-(5-{[2,6-bis (trifluoromethyl)phenyl] methoxy}pyrimidin-2-yl) imidazol-4-yl]methanol (77.81 mg, 0.19 mmol, 12.44%) as a white solid (Purity: >95%).

### Example 370

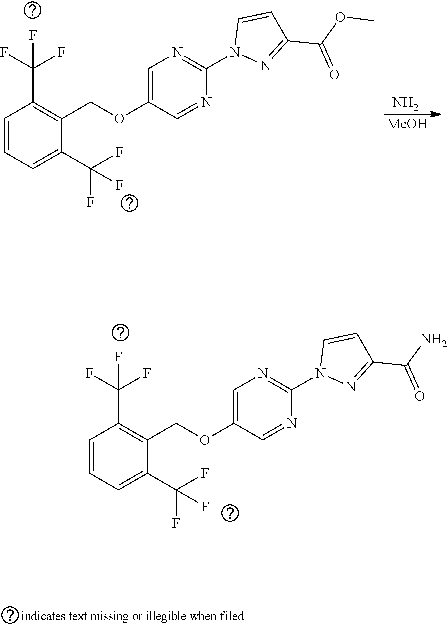

A solution of methyl 1-(5-{[2,6-bis (trifluoromethyl)phenyl] methoxy}pyrimidin-2-yl) pyrazole-3-carboxylate (204 mg, 0.46 mmol, 1 eq) in ammonia (7N in MeOH) (4 mL, 7M in 28 mmol, 61.26 eq) in a sealed tube was heated to 80° C., where it was maintained for ca 4 h. LCMS shows a trace to starting material still remaining. Further ammonia (7N in MeOH), (0.5 mL) was added then heating maintained at 80° C. for an additional ca 2.5 h. The reaction was cooled to rt, where it was stirred overnight. LCMS suggests the reaction has gone nearly to completion. The solvent was removed in vacuo then the resultant residue purified by automated flash chromatography (Combiflash 300+, Silica 12 g Gold RediSep column) eluting with 0 to 12% Methanol in Dichloromethane. Fractions 1-3 were combined then the solvents removed in vacuo. The resultant residue was transferred to a submission vial using DCM then the solvent removed under a stream of nitrogen. The sample was dried overnight under vacuum at 45° C. and gave 1-(5-{[2,6-bis (trifluoromethyl)phenyl] methoxy}pyrimidin-2-yl) pyrazole-3-carboxamide (152.05 mg, 0.35 mmol, 77.13%) as a white foam (Purity: >95%).

Characterization data for selected compounds, including 1H-NMR and LC-MS data, is provided below in Table A.

TABLE A
Structures and Data for Compounds 1-370

| Compound | Structure | ¹H NMR | LCMS [M + H] |
|---|---|---|---|
| 1 | 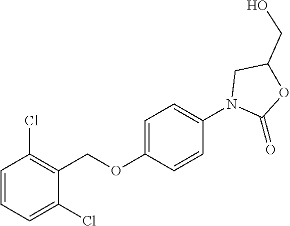 [SMILES: O=C1OC(CO)CN1c1ccc(OCc2c(Cl)cccc2Cl)cc1] | ¹H NMR (400 MHz, DMSO- d6) δ 7.60-7.41 (m, 5H), 7.14-7.05 (m, 2H), 5.22 (d, J = 2.4 Hz, 3H), 4.68 (m, J = 9.7, 6.5, 3.8 Hz, 1H), 4.06 (t, J = 8.9 Hz, 1H), 3.81 (dd, J = 8.7, 6.3 Hz, 1H), 3.67 (m, J = 12.3, 5.4, 3.5 Hz, 1H), 3.56 (m, J = 12.3, 5.6, 4.1 Hz, 1H). | 368 |
| 2 | 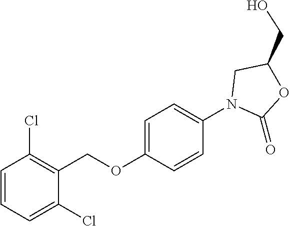 [SMILES: O=C1O[C@H](CO)CN1c1ccc(OCc2c(Cl)cccc2Cl)cc1] | ¹H NMR (400 MHz, DMSO- d6) δ 7.60-7.41 (m, 5H), 7.14-7.05 (m, 2H), 5.22 (d, J = 2.4 Hz, 3H), 4.68 (m, J = 9.7, 6.5, 3.8 Hz, 1H), 4.06 (t, J = 8.9 Hz, 1H), 3.81 (dd, J = 8.7, 6.3 Hz, 1H), 3.67 (m, J = 12.3, 5.4, 3.5 Hz, 1H), 3.56 (m, J = 12.3, 5.6, 4.1 Hz, 1H). | 368 |
| 3 | 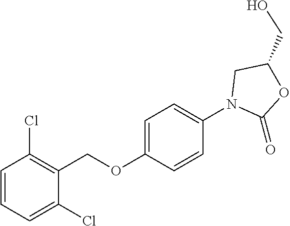 [SMILES: O=C1O[C@@H](CO)CN1c1ccc(OCc2c(Cl)cccc2Cl)cc1] | ¹H NMR (400 MHz, DMSO- d₆) δ 7.60-7.41 (m, 5H), 7.14-7.05 (m, 2H), 5.22 (d, J = 2.4 Hz, 3H), 4.68 (m, J = 9.7, 6.5, 3.8 Hz, 1H), 4.06 (t, J = 8.9 Hz, 1H), 3.81 (dd, J = 8.7, 6.3 Hz, 1H), 3.67 (m, J = 12.3, 5.4, 3.5 Hz, 1H), 3.56 (m, J = 12.3, 5.6, 4.1 Hz, 1H). | 368 |
| 4 | 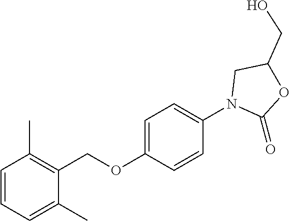 [SMILES: Cc1cccc(C)c1COc1ccc(N2CC(CO)OC2=O)cc1] | 1H NMR (400 MHz, DMSO) δ 7.52-7.48 (m, 2H), 7.18-7.14 (m, J = 1.6 Hz, 1H), 7.10-7.07 (m, 4H), 5.22 (t, J = 5.2 Hz, 1H), 5.03 (s, 2H), 4.70-4.64 (m, 1H), 4.06 (t, J = 9.2 Hz, 1H), 3.83-3.79 (m, 1H), 3.70- 3.64 (m, 1H), 3.58-3.53 (m, 1H), 2.33 (s, 6H). | 328 |
| 5 | 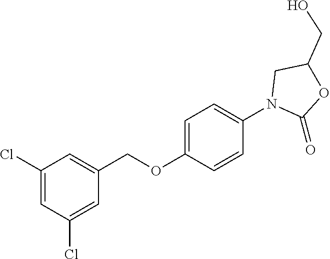 [SMILES: O=C1OC(CO)CN1c1ccc(OCc2cc(Cl)cc(Cl)c2)cc1] | 1H NMR (400 MHz, DMSO) δ 7.58 (t, J = 2.0 Hz, 1H), 7.51-7.47 (m, 4H), 7.07-7.03 (m, 2H), 5.20 (t, J = 6.0 Hz, 1H), 5.13 (s, 2H), 4.69-4.63 (m, 1H), 4.04 (t, J = 9.2 Hz, 1H), 3.81-3.77 (m, 1H), 3.69-3.63 (m, 1H), 3.58-3.52 (m, 1H). | 368 |
| 6 | 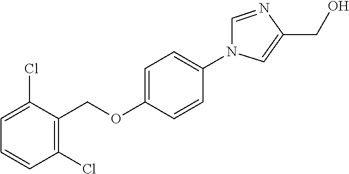 [SMILES: OCc1cn(-c2ccc(OCc3c(Cl)cccc3Cl)cc2)cn1] | 1H NMR (400 MHz, DMSO) δ 7.56 (d, J = 7.8 Hz, 2H), 7.47 (t, J = 9.0 Hz, 3H), 7.09 (s, 1H), 7.01 (d, J = 8.8 Hz, 2H), 5.19 (s, 2H), 4.03- 3.76 (m, J = 2.4 Hz, 2H), 3.53 (dd, J = 8.4, 5.0 Hz, 1H), 3.35 (s, 9H). | 349 |
| 7 | 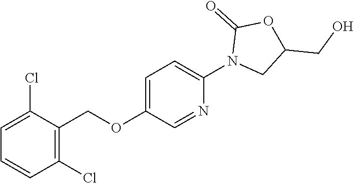 [SMILES: O=C1OC(CO)CN1c1ccc(OCc2c(Cl)cccc2Cl)cn1] | 1H NMR (400 MHz, DMSO) δ 8.21 (d, J = 3.2 Hz, 1H), 8.04 (d, J = 9.2 Hz, 1H), 7.65 (dd, J = 9.2, 3.2 Hz, 1H), 7.58 (d, J = 7.6 Hz, 2H), 7.48 (dd, J = 8.8, 7.2 Hz, 1H), 5.30 (s, 2H), 5.21 (t, J = 5.6 Hz, 1H), 4.72-4.68 (m, 1H), 4.16 (t, J = 9.6 Hz, 1H), 3.95 (dd, J = 10.0, 6.4 Hz, 1H), 3.70-3.66 (m, 1H), 3.59-3.54 (m, 1H). | 369 |
| 8 | 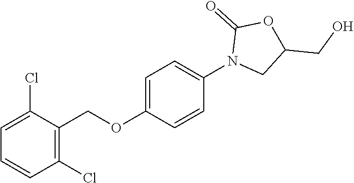 [SMILES: O=C1OC(CO)CN1c1ccc(OCc2c(Cl)cccc2Cl)cc1] | 1H NMR (400 MHz, DMSO) δ 7.49 (dd, J = 14.8, 8.8 Hz, 4H), 7.29 (t, J = 7.6 Hz, 1H), 7.25 (d, J = 8.4 Hz, 2H), 5.22 (t, J = 5.6 Hz, 1H), 4.72-4.66 (m, 1H), 4.07 (t, J = 8.8 Hz, 1H), 3.82 (dd, J = 8.8, 6.4 Hz, 1H), 3.70-3.63 (m, 1H), 3.61-3.51 (m, 1H), 3.13-3.09 (m, 2H), 2.79- 2.75 (m, 2H). | 366 |
| 9 | 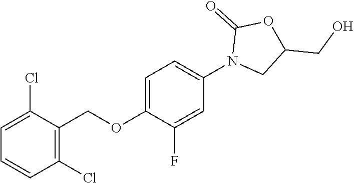 [SMILES: O=C1OC(CO)CN1c1ccc(OCc2c(Cl)cccc2Cl)c(F)c1] | 1H NMR (400 MHz, DMSO) δ 7.63-7.54 (m, 3H), 7.48 (dd, J = 8.8, 7.2 Hz, 1H), 7.38 (t, J = 9.2 Hz, 1H), 7.28-7.25 (m, 1H), 5.29 (s, 2H), 5.22 (t, J = 5.6 Hz, 1H), 4.71-4.67 (m, 1H), 4.06 (t, J = 8.8 Hz, 1H), 3.81 (dd, J = 9.2, 6.4 Hz, 1H), 3.70-3.65 (m, 1H), 3.58-3.53 (m, 1H). | 386 |
| 10 | 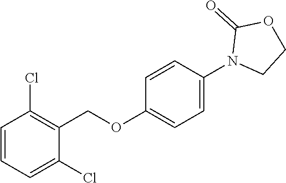 [SMILES: O=C1OCCN1c1ccc(OCc2c(Cl)cccc2Cl)cc1] | 1H NMR (400 MHz, DMSO) δ 7.57 (d, J = 7.6 Hz, 2H), 7.51-7.45 (m, 3H), 7.11-7.09 (m, 2H), 5.22 (s, 2H), 4.42 (t, J = 7.6 Hz, 2H), 4.04 (t, J = 9.2 Hz, 2H). | 338 |
| 11 | 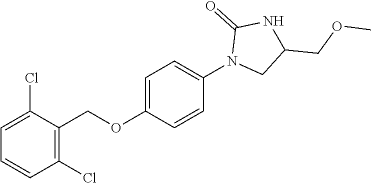 [SMILES: COCC1CN(c2ccc(OCc3c(Cl)cccc3Cl)cc2)C(=O)N1] | 1H NMR (400 MHz, DMSO) δ 7.56 (d, J = 7.8 Hz, 2H), 7.47 (t, J = 9.0 Hz, 3H), 7.09 (s, 1H), 7.01 (d, J = 8.8 Hz, 2H), 5.19 (s, 2H), 4.03- 3.76 (m, 2H), 3.53 (dd, J = 8.4, 5.0 Hz, 1H), 3.35 (s, 9H). | 381 |
| 12 | 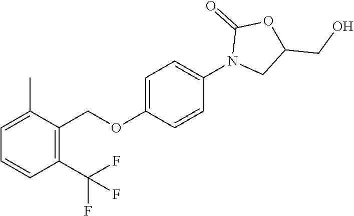 [SMILES: Cc1cccc(C(F)(F)F)c1COc1ccc(N2CC(CO)OC2=O)cc1] | 1H NMR (400 MHz, DMSO) δ 7.63 (t, J = 8.0 Hz, 2H), 7.55-7.49 (m, 3H), 7.11-7.08 (m, 2H), 5.21 (t, J = 6.0 Hz, 1H), 5.10 (s, 2H), 4.70-4.65 (m, 1H), 4.06 (t, J = 8.8 Hz, 1H), 3.81 (dd, J = 8.4, 6.4 Hz, 1H), 3.70-3.65 (m, 1H), 3.59-3.54 (m, 1H), 2.42 (s, 3H). | 382 |
| 13 | 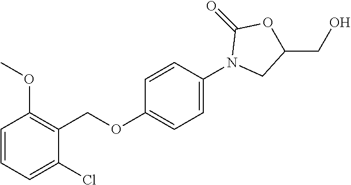 [SMILES: COc1cccc(Cl)c1COc1ccc(N2CC(CO)OC2=O)cc1] | 1H NMR (400 MHz, DMSO) δ 7.48 (d, J = 8.7 Hz, 2H), 7.41 (t, J = 8.2 Hz, 1H), 7.25-6.85 (m, 4H), 5.29- 5.17 (m, 1H), 5.09 (s, 2H), 4.67 (s, 1H), 4.05 (t, J = 8.8 Hz, 1H), 3.91-3.75 (m, 4H), 3.67 (d, J = 12.2 Hz, 1H), 3.56 (d, J = 12.0 Hz, 1H). | 364 |
| 14 | 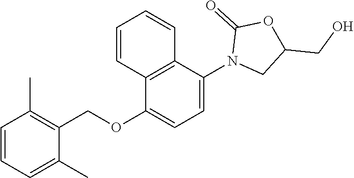 [SMILES: Cc1cccc(C)c1COc1ccc(N2CC(CO)OC2=O)c2ccccc12] | 1H NMR (400 MHz, DMSO) δ 7.99 (dd, J = 44.4, 8.2 Hz, 2H), 7.66-7.44 (m, 3H), 7.32-7.17 (m, 2H), 7.13 (d, J = 7.6 Hz, 2H), 5.39 (t, J = 5.6 Hz, 1H), 5.28 (s, 2H), 4.83 (dt, J = 9.2, 4.6 Hz, 1H), 4.07 (t, J = 8.8 Hz, 1H), 3.87-3.71 (m, 2H), 3.65 (m, J = 12.2, 5.8, 3.8 Hz, 1H), 2.38 (s, 6H). | 378 |
| 15 | 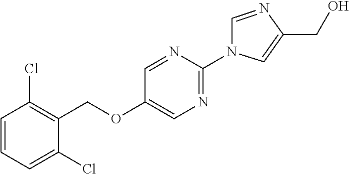 [SMILES: OCc1cn(-c2ncc(OCc3c(Cl)cccc3Cl)cn2)cn1] | 1H NMR (399 MHz, DMSO) δ 8.78 (s, 2H), 8.43 (d, J = 1.4 Hz, 1H), 7.74- 7.69 (m, 1H), 7.61 (d, J = 8.7 Hz, 2H), 7.52 (dd, J = 9.0, 7.1 Hz, 1H), 5.47 (s, 2H), 5.04 (t, J = 5.7 Hz, 1H), 4.46- 4.40 (m, 2H). | 351 |
| 16 | 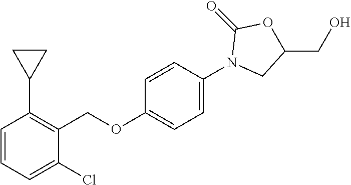 [SMILES: O=C1OC(CO)CN1c1ccc(OCc2c(Cl)cccc2C2CC2)cc1] | 1H NMR (400 MHz, MeOD) δ 7.56-7.42 (m, 2H), 7.32-7.20 (m, 2H), 7.13-6.99 (m, 3H), 5.39 (s, 2H), 4.73 (m, J = 9.8, 7.0, 3.8 Hz, 1H), 4.12 (t, J = 9.0 Hz, 1H), 3.92 (dd, J = 8.8, 6.6 Hz, 1H), 3.85 (dd, J = 12.6, 3.4 Hz, 1H), 3.70 (dd, J = 12.6, 4.2 Hz, 1H), 2.15-2.05 (m, 1H), 1.30 (s, 1H), 0.97-0.84 (m, 2H), 0.76-0.64 (m, 2H). | 374 |
| 17 | 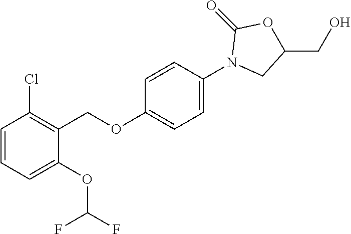 [SMILES: O=C1OC(CO)CN1c1ccc(OCc2c(Cl)cccc2OC(F)F)cc1] | 1H NMR (400 MHz, DMSO) δ 7.55-7.46 (m, 4H), 7.30-7.28 (m, 1H), 7.28 (t, J = 73.2 Hz, 1H), 7.08 (m, 2H), 5.22 (t, J = 5.6 Hz, 1H), 5.11 (s, 2H), 4.69- 4.66 (m, 1H), 4.06 (t, J = 9.2 Hz, 1H), 3.81 (dd, J = 8.8, 6.4 Hz, 1H), 3.70-3.65 (m, 1H), 3.59-3.54 (m, 1H). | 400 |
| 19 | 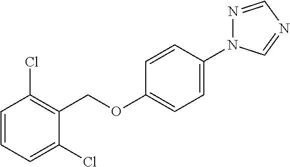 [SMILES: Clc1cccc(Cl)c1COc1ccc(-n2cncn2)cc1] | 1H NMR (400 MHz, DMSO) δ 9.20 (s, 1H), 8.20 (s, 1H), 7.84-7.77 (m, 2H), 7.59 (d, J = 8.6 Hz, 2H), 7.49 (dd, J = 8.9, 7.2 Hz, 1H), 7.29-7.23 (m, 2H), 5.30 (s, 2H). | 320 |
| 20 | 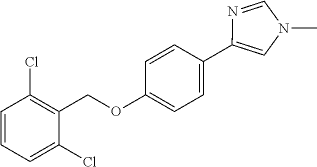 [SMILES: Cn1cnc(-c2ccc(OCc3c(Cl)cccc3Cl)cc2)c1] | 1H NMR (400 MHz, DMSO) δ 7.69-7.67 (m, 2H), 7.59-7.56 (m, 3H), 7.49-7.45 (m, 2H), 7.05- 7.03 (m, 2H), 5.23 (s, 2H), 3.67 (s, 3H). | 333 |
| 21 | 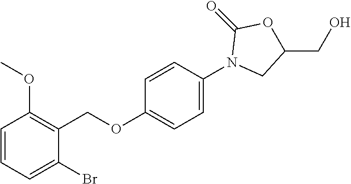 [SMILES: COc1cccc(Br)c1COc1ccc(N2CC(CO)OC2=O)cc1] | 1H NMR (400 MHz, DMSO) δ 7.48 (d, J = 9.1 Hz, 2H), 7.34 (t, J = 8.1 Hz, 1H), 7.27 (dd, J = 8.0, 1.0 Hz, 1H), 7.12 (d, J = 7.5 Hz, 1H), 7.09-7.00 (m, 2H), 5.21 (t, J = 5.6 Hz, 1H), 5.09 (s, 2H), 4.67 (dd, J = 9.0, 6.0 Hz, 1H), 4.06 (t, J = 8.9 Hz, 1H), 3.91-3.73 (m, 4H), 3.73- 3.61 (m, 1H), 3.62-3.49 (m, 1H). | 408 |
| 22 | 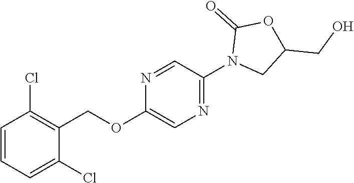 [SMILES: O=C1OC(CO)CN1c1cnc(OCc2c(Cl)cccc2Cl)cn1] | 1H NMR (400 MHz, DMSO) δ 8.91 (d, J = 1.2 Hz, 1H), 8.21 (d, J = 1.2 Hz, 1H), 7.58 (d, J = 7.8 Hz, 2H), 7.49 (dd, J = 8.8, 7.2 Hz, 1H), 5.54 (s, 2H), 5.24 (t, J = 5.4 Hz, 1H), 4.77 (dd, J = 9.0, 5.8 Hz, 1H), 4.15 (t, J = 9.4 Hz, 1H), 3.94 (dd, J = 9.8, 6.2 Hz, 1H), 3.77-3.50 (m, 2H). | 370 |
| 23 | 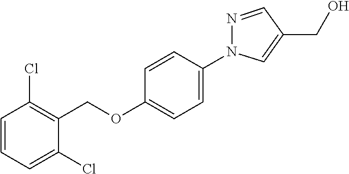 [SMILES: OCc1cnn(-c2ccc(OCc3c(Cl)cccc3Cl)cc2)c1] | 1H NMR (400 MHz, DMSO) δ 8.29 (s, 1H), 7.74 (d, J = 9.0 Hz, 2H), 7.63 (s, 1H), 7.58 (d, J = 7.7 Hz, 2H), 7.48 (dd, J = 8.9, 7.2 Hz, 1H), 7.18 (d, J = 9.1 Hz, 2H), 5.27 (s, 2H), 4.97 (t, J = 5.4 Hz, 1H), 4.43 (d, J = 5.4 Hz, 2H). | 349 |
| 24 | 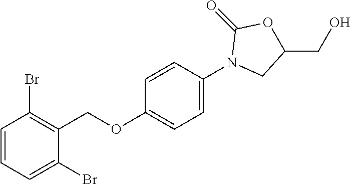 [SMILES: O=C1OC(CO)CN1c1ccc(OCc2c(Br)cccc2Br)cc1] | ¹H NMR (400 MHz, DMSO) δ 7.75 (d, J = 8.1 Hz, 2H), 7.51 (d, J = 9.0 Hz, 2H), 7.29 (t, J = 8.0 Hz, 1H), 7.10 (d, J = 9.0 Hz, 2H), 5.23 (d, J = 11.5 Hz, 2H), 4.66 (d, J = 7.4 Hz, 1H), 4.06 (t, J = 8.9 Hz, 1H), 3.81 (dd, J = 8.7, 6.3 Hz, 1H), 3.67 (dt, J = 12.1, 5.0 Hz, 1H), 3.62-3.46 (m, 1H). | 456 |
| 25 | 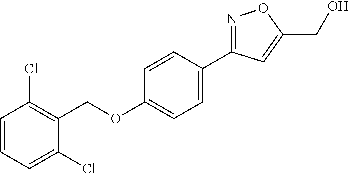 [SMILES: OCc1cc(-c2ccc(OCc3c(Cl)cccc3Cl)cc2)no1] | ¹H NMR (400 MHz, DMSO) δ 7.84 (d, J = 8.8 Hz, 2H), 7.59 (d, J = 7.6 Hz, 2H), 7.49 (dd, J = 8.8, 7.2 Hz, 1H), 7.20 (d, J = 8.8 Hz, 2H), 6.89 (s, 1H), 5.72 (t, J = 6.0 Hz, 1H), 5.30 (s, 2H), 4.60 (d, J = 6.0 Hz, 2H). | 350 |
| 26 | 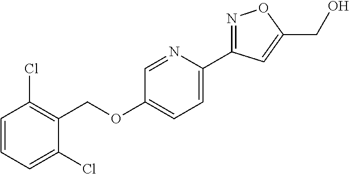 [SMILES: OCc1cc(-c2ccc(OCc3c(Cl)cccc3Cl)cn2)no1] | 1H NMR (400 MHz, DMSO) δ 8.53 (d, J = 2.8 Hz, 1H), 8.01 (d, J = 8.8 Hz, 1H), 7.74 (dd, J = 8.8, 2.8 Hz, 1H), 7.61 (d, J = 7.8 Hz, 2H), 7.51 (dd, J = 9.2, 7.2 Hz, 1H), 6.83 (s, 1H), 5.72 (t, J = 6.0 Hz, 1H), 5.40 (s, 2H), 4.63 (d, J = 6.0 Hz, 2H). | 351 |
| 27 | 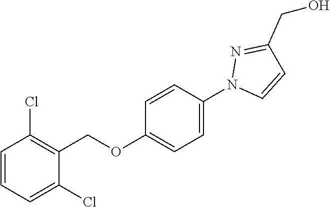 [SMILES: OCc1ccn(-c2ccc(OCc3c(Cl)cccc3Cl)cc2)n1] | 1H NMR (400 MHz, DMSO) δ 8.33 (d, J = 2.4 Hz, 1H), 7.74 (d, J = 9.0 Hz, 2H), 7.63-7.53 (m, 2H), 7.49 (dd, J = 8.9, 7.2 Hz, 1H), 7.18 (d, J = 9.1 Hz, 2H), 6.45 (d, J = 2.4 Hz, 1H), 5.27 (s, 2H), 5.15 (t, J = 5.8 Hz, 1H), 4.50 (d, J = 5.8 Hz, 2H). | 349 |
| 28 | 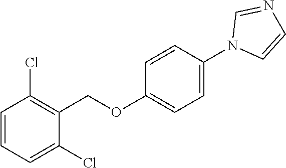 [SMILES: Clc1cccc(Cl)c1COc1ccc(-n2ccnc2)cc1] | 1H NMR (400 MHz, DMSO) δ 8.16 (s, 1H), 7.67 (t, J = 1.4 Hz, 1H), 7.63- 7.56 (m, 4H), 7.49 (dd, J = 8.9, 7.2 Hz, 1H), 7.25-7.18 (m, 2H), 7.09 (s, 1H), 5.29 (s, 2H). | 319 |
| 29 | 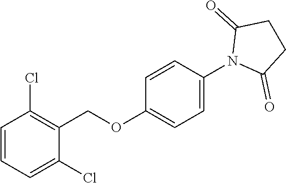 [SMILES: O=C1CCC(=O)N1c1ccc(OCc2c(Cl)cccc2Cl)cc1] | 1H NMR (400 MHz, DMSO) δ 7.66-7.54 (m, 2H), 7.49 (dd, J = 8.8, 7.2 Hz, 1H), 7.27-7.09 (m, 4H), 5.26 (s, 2H), 2.77 (s, 4H). | 350 |
| 30 | 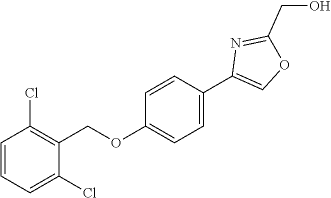 [SMILES: OCc1nc(-c2ccc(OCc3c(Cl)cccc3Cl)cc2)co1] | ¹H NMR (400 MHz, DMSO) δ 8.48 (s, 1H), 7.80-7.65 (m, 2H), 7.64-7.53 (m, 2H), 7.49 (dd, J = 8.8, 7.2 Hz, 1H), 7.20-7.00 (m, 2H), 5.71 (t, J = 6.4 Hz, 1H), 5.27 (s, 2H), 4.55 (d, J = 6.2 Hz, 2H). | 350 |
| 31 | 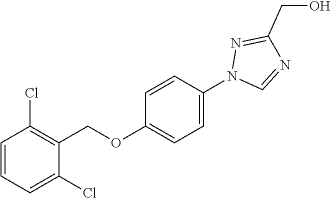 [SMILES: OCc1ncn(-c2ccc(OCc3c(Cl)cccc3Cl)cc2)n1] | 1H NMR (400 MHz, DMSO) δ 9.11 (s, 1H), 7.83- 7.70 (m, 2H), 7.64-7.54 (m, 2H), 7.49 (dd, J = 8.8, 7.2 Hz, 1H), 7.31-7.16 (m, 2H), 5.29 (s, 2H), 4.52 (s, 2H). | 350 |
| 32 | 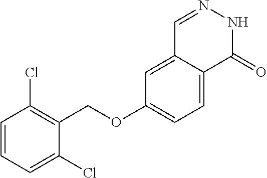 [SMILES: O=c1[nH]ncc2cc(OCc3c(Cl)cccc3Cl)ccc12] | ¹H NMR (400 MHz, DMSO) δ 12.57 (s, 1H), 8.29 (s, 1H), 8.16 (d, J = 8.8 Hz, 1H), 7.65 (d, J = 2.0 Hz, 1H), 7.61 (d, J = 7.6 Hz, 2H), 7.54- 7.47 (m, 2H), 5.41 (s, 2H). | 321 |
| 33 | 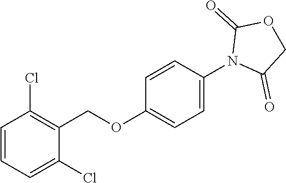 [SMILES: O=C1COC(=O)N1c1ccc(OCc2c(Cl)cccc2Cl)cc1] | ¹H NMR (400 MHz, DMSO) δ 7.59 (d, J = 7.8 Hz, 2H), 7.49 (dd, J = 8.8, 7.2 Hz, 1H), 7.36 (d, J = 8.8 Hz, 2H), 7.22 (d, J = 8.8 Hz, 2H), 5.28 (s, 2H), 4.95 (s, 2H). | 352 |
| 34 | 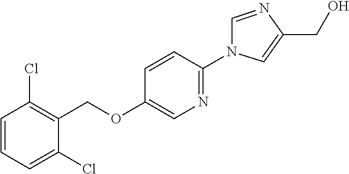 [SMILES: OCc1cn(-c2ccc(OCc3c(Cl)cccc3Cl)cn2)cn1] | 1H NMR (400 MHz, DMSO) δ 9.19 (s, 1H), 8.46 (d, J = 2.2 Hz, 1H), 8.00- 7.84 (m, 2H), 7.55 (dt, J = 8.8, 7.6 Hz, 4H), 5.42 (s, 2H), 4.66 (s, 2H). | 350 |
| 35 | 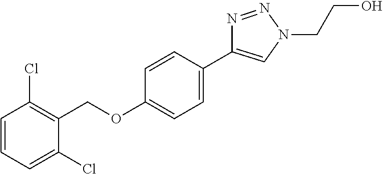 [SMILES: OCCn1cc(-c2ccc(OCc3c(Cl)cccc3Cl)cc2)nn1] | 1H NMR (400 MHz, DMSO) δ 8.43 (s, 1H), 7.81 (d, J = 8.8 Hz, 2H), 7.58 (d, J = 7.6 Hz, 2H), 7.49 (dd, J = 8.8, 7.2 Hz, 1H), 7.16 (d, J = 8.8 Hz, 2H), 5.28 (s, 2H), 4.45 (t, J = 5.2 Hz, 2H), 3.83 (t, J = 5.2 Hz, 2H). | 364 |
| 36 | 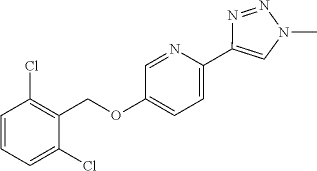 [SMILES: Cn1cc(-c2ccc(OCc3c(Cl)cccc3Cl)cn2)nn1] | 1H NMR (400 MHz, DMSO) δ 8.46 (s, 1H), 8.42 (d, J = 2.8 Hz, 1H), 8.00 (d, J = 8.8 Hz, 1H), 7.68 (dd, J = 8.8, 3.0 Hz, 1H), 7.59 (d, J = 7.8 Hz, 2H), 7.50 (dd, J = 8.8, 7.2 Hz, 1H), 5.36 (s, 2H), 4.10 (s, 3H). | 335 |
| 37 | 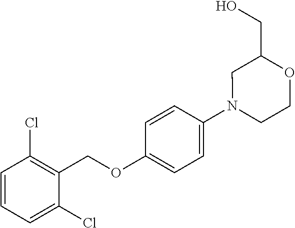 [SMILES: OCC1CN(c2ccc(OCc3c(Cl)cccc3Cl)cc2)CCO1] | 1H NMR (400 MHz, DMSO) δ 7.56 (d, J = 8.8 Hz, 1H), 7.54 (d, J = 7.2 Hz, 1H), 7.45 (dd, J = 8.8, 7.2 Hz, 1H), 6.96 (d, J = 9.2 Hz, 2H), 6.90 (d, J = 9.2 Hz, 2H), 5.15 (s, 2H), 4.75 (br s, 1H), 3.94- 3.90 (m, 1H), 3.63 (td, J = 11.2, 2.4 Hz, 1H), 3.59- 3.53 (m, 1H), 3.51-3.38 (m, 4H), 2.60 (td, J = 11.6, 3.2 Hz, 1H), 2.38-2.23 (m, 1H). | 368 |
| 38 | 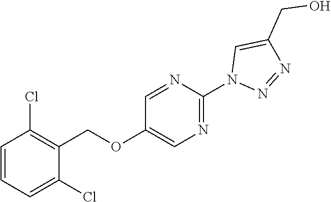 [SMILES: OCc1cn(-c2ncc(OCc3c(Cl)cccc3Cl)cn2)nn1] | 1H NMR (400 MHz, DMSO) δ 8.58 (s, 1H), 8.43 (d, J = 2.8 Hz, 1H), 8.08 (d, J = 8.8 Hz, 1H), 7.89 (dd, J = 9.0, 3.0 Hz, 1H), 7.67-7.56 (m, 2H), 7.51 (dd, J = 8.8, 7.2 Hz, 1H), 5.42 (s, 2H), 5.32 (t, J = 5.6 Hz, 1H), 4.62 (d, J = 5.0 Hz, 2H). | 351 |
| 39 | 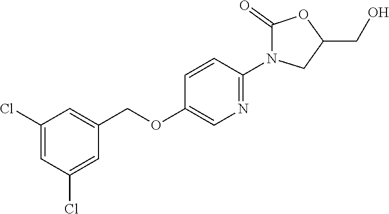 [SMILES: O=C1OC(CO)CN1c1ccc(OCc2cc(Cl)cc(Cl)c2)cn1] | 1H NMR (399 MHz, DMSO) δ 8.09 (dd, J = 3.0, 0.7 Hz, 1H), 7.95 (dd, J = 9.1, 0.6 Hz, 1H), 7.56-7.49 (m, 2H), 7.46 (d, J = 1.9 Hz, 2H), 5.15-5.08 (m, 3H), 4.62 (ddt, J = 7.4, 6.3, 3.7 Hz, 1H), 4.07 (dd, J = 10.0, 9.1 Hz, 1H), 3.87 (dd, J = 10.0, 6.3 Hz, 1H), 3.60 (ddd, J = 12.3, 5.5, 3.3 Hz, 1H), 3.49 (ddd, J = 12.4, 5.8, 4.1 Hz, 1H). Hz, 1H), 3.49 (ddd, J = 12.4, 5.8, 4.1 Hz, 1H) | 369 |
| 40 | 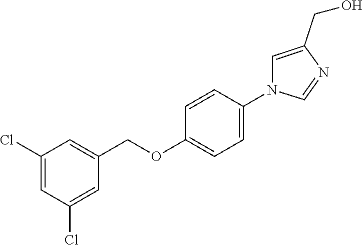 [SMILES: OCc1cn(-c2ccc(OCc3cc(Cl)cc(Cl)c3)cc2)cn1] | 1H NMR (400 MHz, DMSO) δ 8.06 (s, 1H), 7.58 (d, J = 1.8 Hz, 1H), 7.55 (s, 1H), 7.53 (d, J = 2.0 Hz, 3H), 7.47 (s, 1H), 7.13 (d, J = 9.0 Hz, 2H), 5.18 (s, 2H), 4.38 (s, 2H). | 349 |
| 41 | 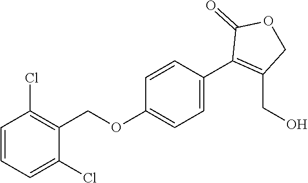 [SMILES: O=C1OCC(CO)=C1c1ccc(OCc2c(Cl)cccc2Cl)cc1] | ¹H NMR (400 MHz, DMSO) δ 7.65-7.54 (m, 2H), 7.54- 7.38 (m, 3H), 7.16 (d, J = 8.8 Hz, 2H), 5.46 (t, J = 5.6 Hz, 1H), 5.28 (s, 2H), 5.02 (s, 2H), 4.59 (d, J = 5.6 Hz, 2H). | 365 |
| 43 | 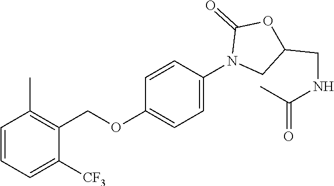 [SMILES: CC(=O)NCC1CN(c2ccc(OCc3c(C)cccc3C(F)(F)F)cc2)C(=O)O1] | 1H NMR (400 MHz, DMSO) δ 8.25 (t, J = 6.0 Hz, 1H), 7.63 (t, J = 8.0 Hz, 2H), 7.53 (t, J = 7.6 Hz, 1H), 7.48- 7.45 (m, 2H), 7.10-7.08 (m, 2H), 5.10 (s, 2H), 4.73- 4.67 (m, 1H), 4.10 (t, J = 8.8 Hz, 1H), 3.72 (dd, J = 8.8, 6.4 Hz, 1H), 3.41 (t, J = 5.2 Hz, 2H), 2.42 (s, 3H), 1.84 (s, 3H). | 423 |
| 44 | 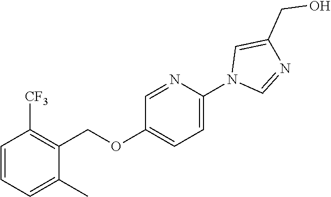 [SMILES: Cc1cccc(C(F)(F)F)c1COc1ccc(-n2cnc(CO)c2)nc1] | 1H NMR (399 MHz, DMSO) δ 8.36 (d, J = 1.4 Hz, 1H), 8.31 (t, J = 1.9 Hz, 1H), 7.82-7.74 (m, 2H), 7.71 (d, J = 1.3 Hz, 1H), 7.65 (t, J = 7.6 Hz, 2H), 7.55 (t, J = 7.8 Hz, 1H), 5.25 (s, 2H), 4.99 (s, 1H), 4.41 (s, 2H), 2.44 (s, 3H). | 364 |
| 45 | 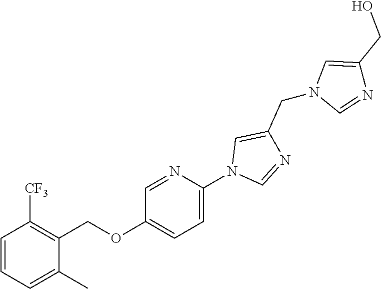 [SMILES: Cc1cccc(C(F)(F)F)c1COc1ccc(-n2cnc(Cn3cnc(CO)c3)c2)nc1] | 1H NMR (399 MHz, DMSO) δ 8.43 (dd, J = 8.3, 1.4 Hz, 1H), 8.31 (t, J = 1.8 Hz, 1H), 7.84 (dd, J = 10.4, 1.4 Hz, 1H), 7.80 (d, J = 1.8 Hz, 2H), 7.64 (q, J = 11.3, 9.5 Hz, 3H), 7.55 (t, J = 7.7 Hz, 1H), 6.91 (d, J = 93.3 Hz, 1H), 5.35 (s, 1H), 5.25 (s, 2H), 5.10 (d, J = 33.2 Hz, 2H), 4.76 (s, 0H), 4.41 (dd, J = 91.8, 4.4 Hz, 2H), 2.44 (s, 3H). | 444 |
| 46 | 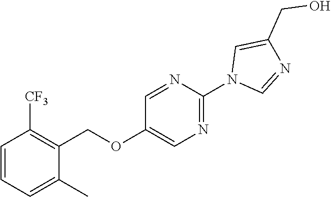 [SMILES: Cc1cccc(C(F)(F)F)c1COc1cnc(-n2cnc(CO)c2)nc1] | 1H NMR (399 MHz, DMSO) δ 8.77 (s, 2H), 8.42 (d, J = 1.3 Hz, 1H), 7.71 (q, J = 1.1 Hz, 1H), 7.66 (t, J = 7.3 Hz, 2H), 7.57 (t, J = 7.8 Hz, 1H), 5.35 (s, 2H), 5.04 (t, J = 5.7 Hz, 1H), 4.43 (dd, J = 5.7, 1.0 Hz, 2H), 2.45 (s, 3H) | 365 |
| 47 | 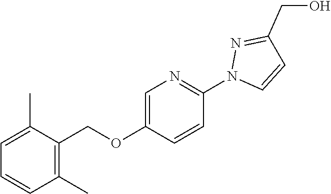 [SMILES: Cc1cccc(C)c1COc1ccc(-n2ccc(CO)n2)nc1] | 1H NMR (399 MHz, DMSO) δ 8.44 (d, J = 2.5 Hz, 1H), 8.25 (d, J = 2.9 Hz, 1H), 7.83 (d, J = 8.9 Hz, 1H), 7.73 (dd, J = 9.0, 2.9 Hz, 1H), 7.18 (dd, J = 8.2, 6.7 Hz, 1H), 7.09 (d, J = 7.5 Hz, 2H), 6.49 (d, J = 2.5 Hz, 1H), 5.17 (s, 2H), 4.52 (s, 2H), 2.35 (s, 6H). | 310 |
| 48 | 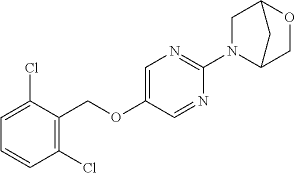 [SMILES: Clc1cccc(Cl)c1COc1cnc(N2CC3CC2CO3)nc1] | 1H NMR (399 MHz, DMSO) δ 8.28 (s, 2H), 7.59- 7.54 (m, 2H), 7.47 (dd, J = 9.0, 7.1 Hz, 1H), 5.24 (s, 2H), 4.82 (dt, J = 2.5, 1.3 Hz, 1H), 4.66-4.58 (m, 1H), 3.71 (ddd, J = 56.9, 7.3, 1.2 Hz, 2H), 3.54-3.35 (m, 2H), 2.07-1.71 (m, 2H) | 352 |
| 49 | 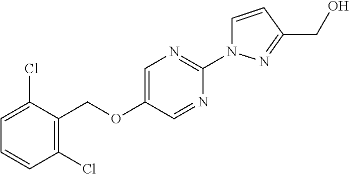 [SMILES: OCc1ccn(-c2ncc(OCc3c(Cl)cccc3Cl)cn2)n1] | 1H NMR (399 MHz, DMSO) δ 8.74 (s, 2H), 8.49 (d, J = 2.6 Hz, 1H), 7.65- 7.57 (m, 2H), 7.51 (dd, J = 9.0, 7.1 Hz, 1H), 6.52 (d, J = 2.6 Hz, 1H), 5.46 (s, 2H), 5.24 (s, 1H), 4.52 (s, 2H) | 351 |
| 50 | 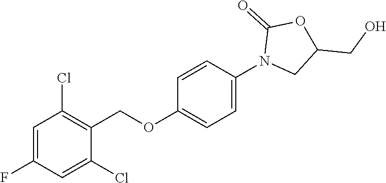 [SMILES: O=C1OC(CO)CN1c1ccc(OCc2c(Cl)cc(F)cc2Cl)cc1] | 1H NMR (400 MHz, DMSO) δ 7.65 (d, J = 7.5 Hz, 2H), 7.52 (d, J = 7.1 Hz, 2H), 7.10 (d, J = 7.0 Hz, 2H), 5.20 (s, 3H), 4.69 (s, 1H), 4.07 (s, 1H), 3.82 (s, 1H), 3.67 (s, 1H), 3.54 (d, J = 37.6 Hz, 1H). | 386 |
| 51 | 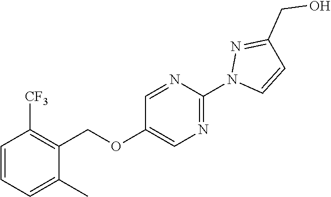 [SMILES: Cc1cccc(C(F)(F)F)c1COc1cnc(-n2ccc(CO)n2)nc1] | 1H NMR (399 MHz, DMSO) δ 8.74 (s, 2H), 8.49 (d, J = 2.6 Hz, 1H), 7.66 (dd, J = 7.8, 6.4 Hz, 2H), 7.56 (t, J = 7.7 Hz, 1H), 6.52 (d, J = 2.6 Hz, 1H), 5.35 (s, 2H), 5.24 (t, J = 5.9 Hz, 1H), 4.52 (d, J = 5.6 Hz, 2H), 2.46 (s, 3H). | 365 |
| 52 | 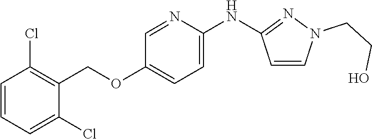 [SMILES: OCCn1ccc(Nc2ccc(OCc3c(Cl)cccc3Cl)cn2)n1] | 1H NMR (399 MHz, DMSO) δ 9.00 (s, 1H), 7.94 (d, J = 3.2 Hz, 1H), 7.58-7.53 (m, 2H), 7.49 (d, J = 2.2 Hz, 1H), 7.46 (dd, J = 8.9, 7.2 Hz, 1H), 7.38 (dd, J = 9.0, 3.1 Hz, 1H), 7.25 (d, J = 9.0 Hz, 1H), 6.22 (d, J = 2.2 Hz, 1H), 5.21 (s, 2H), 4.83 (t, J = 5.4 Hz, 1H), 4.00 (t, J = 5.7 Hz, | 379 |
| | | 2H), 3.71 (q, J = 5.6 Hz, 2H). | |
| 53 | 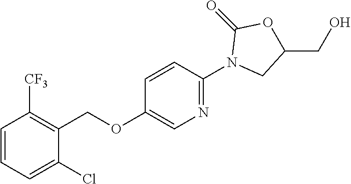 [SMILES: O=C1OC(CO)CN1c1ccc(OCc2c(Cl)cccc2C(F)(F)F)cn1] | 1H NMR (399 MHz, DMSO) δ 8.21 (dd, J = 3.0, 0.7 Hz, 1H), 8.05 (dd, J = 9.1, 0.7 Hz, 1H), 7.94 (dd, J = 8.2, 1.2 Hz, 1H), 7.85 (dd, J = 8.1, 1.2 Hz, 1H), 7.74-7.62 (m, 2H), 5.26 (d, J = 1.4 Hz, 2H), 5.19 (t, J = 5.6 Hz, 1H), 4.70 (ddt, J = 7.4, 6.3, 3.7 Hz, 1H), 4.16 (dd, J = 10.0, 9.1 Hz, 1H), 3.96 (dd, J = 10.1, 6.3 Hz, 1H), 3.68 (ddd, J = 12.4, 5.5, 3.3 Hz, 1H), 3.57 (ddd, J = 12.4, | 403 |
| | | 5.8, 4.1 Hz, 1H) | |
| 54 | 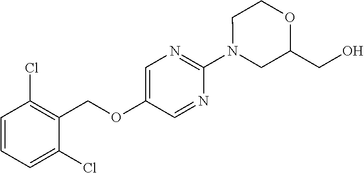 [SMILES: OCC1CN(c2ncc(OCc3c(Cl)cccc3Cl)cn2)CCO1] | 1H NMR (399 MHz, DMSO) δ 8.32 (s, 2H), 7.61- 7.53 (m, 2H), 7.47 (dd, J = 8.9, 7.1 Hz, 1H), 5.25 (s, 2H), 4.78 (t, J = 5.7 Hz, 1H), 4.44 (dt, J = 12.9, 2.0 Hz, 1H), 4.26 (dp, J = 13.1, 1.5 Hz, 1H), 3.91 (ddd, J = 11.4, 3.6, 1.4 Hz, 1H), 3.55-3.34 (m, 4H), 2.90 (ddd, J = 13.1, 11.8, 3.6 Hz, 1H), 2.68-2.57 | 370 |
| | | (m, 1H) | |
| 55 | 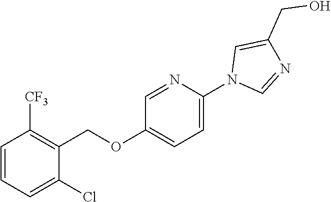 [SMILES: OCc1cn(-c2ccc(OCc3c(Cl)cccc3C(F)(F)F)cn2)cn1] | 1H NMR (399 MHz, DMSO) δ 8.36 (d, J = 1.4 Hz, 1H), 8.31 (t, J = 1.8 Hz, 1H), 7.95 (dd, J = 8.2, 1.2 Hz, 1H), 7.87 (dd, J = 8.0, 1.2 Hz, 1H), 7.84-7.75 (m, 2H), 7.77-7.67 (m, 2H), 5.33 (s, 2H), 4.99 (t, J = 5.6 Hz, 1H), 4.41 (d, J = 5.4 Hz, 2H), 2.55 (s, 2H) | 384 |
| 56 | 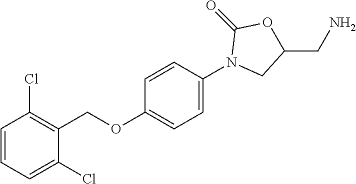 [SMILES: NCC1CN(c2ccc(OCc3c(Cl)cccc3Cl)cc2)C(=O)O1] | 1H NMR (400 MHz, DMSO) δ 7.56 (d, J = 7.8 Hz, 2H), 7.53-7.43 (m, 3H), 7.13-7.06 (m, 2H), 5.22 (s, 2H), 4.74-4.49 (m, 1H), 4.06 (dt, J = 17.8, 8.8 Hz, 1H), 3.82 (m, J = 19.0, 8.8, 6.3 Hz, 1H), 3.26 (d, J = 16.4 Hz, 2H), 2.82 (m, J = 13.6, 5.0 Hz, 2H). | 367 |
| 57 | 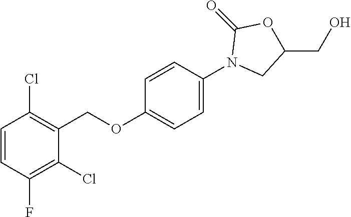 [SMILES: O=C1OC(CO)CN1c1ccc(OCc2c(Cl)ccc(F)c2Cl)cc1] | 1H NMR (400 MHz, DMSO) δ 7.72-7.46 (m, 4H), 7.10 (d, J = 9.1 Hz, 2H), 5.34-5.11 (m, 3H), 4.67 (td, J = 9.8, 3.8 Hz, 1H), 4.13- 4.01 (m, 1H), 3.81 (dd, J = 8.7, 6.4 Hz, 1H), 3.67 (m, J = 12.2, 5.5, 3.5 Hz, 1H), 3.56 (m, J = 12.3, 5.6, 4.3 Hz, 1H). | 387 |
| 58 | 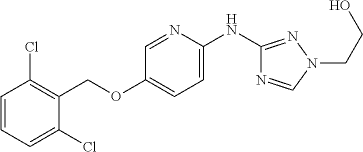 [SMILES: OCCn1cnc(Nc2ccc(OCc3c(Cl)cccc3Cl)cn2)n1] | 1H NMR (399 MHz, DMSO) δ 9.31 (s, 1H), 8.20 (s, 1H), 8.03-7.97 (m, 1H), 7.84 (d, J = 9.2 Hz, 1H), 7.57 (d, J = 1.1 Hz, 1H), 7.55 (s, 1H), 7.52-7.42 (m, 2H), 5.24 (s, 2H), 4.94 (t, J = 5.3 Hz, 1H), 4.10 (t, J = 5.4 Hz, 2H), 3.73 (q, J = 5.2 Hz, 2H) | 380 |
| 59 | 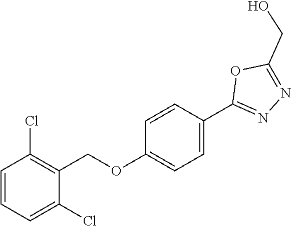 [SMILES: OCc1nnc(-c2ccc(OCc3c(Cl)cccc3Cl)cc2)o1] | 1H NMR (400 MHz, DMSO) δ 8.04-7.92 (m, 2H), 7.66-7.55 (m, 2H), 7.50 (dd, J = 8.8, 7.2 Hz, 1H), 7.37-7.18 (m, 2H), 5.93 (t, J = 6.2 Hz, 1H), 5.34 (s, 2H), 4.71 (d, J = 6.2 Hz, 2H). | 351 |
| 60 | 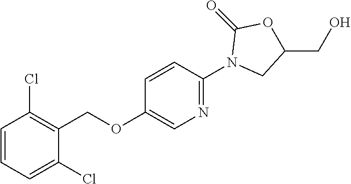 [SMILES: O=C1OC(CO)CN1c1ccc(OCc2c(Cl)cccc2Cl)cn1] | 1H NMR (400 MHz, DMSO) δ 8.18 (d, J = 1.9 Hz, 1H), 8.01 (d, J = 8.6 Hz, 1H), 7.67 (dd, J = 8.6, 2.3 Hz, 1H), 7.46 (d, J = 8.0 Hz, 2H), 7.33-7.26 (m, 1H), 5.20 (s, 1H), 4.71 (td, J = 9.6, 3.6 Hz, 1H), 4.16 (t, J = 9.6 Hz, 1H), 3.95 (dd, J = 10.1, 6.3 Hz, 1H), 3.69 (d, J = 10.7 Hz, 1H), 3.57 (d, J = 11.2 Hz, 1H), 3.19-3.09 (m, 2H), 2.86- 2.77 (m, 2H). | 369 |
| 61 | 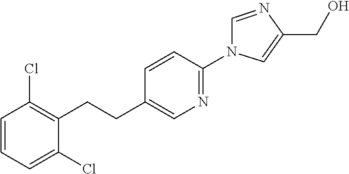 [SMILES: OCc1cn(-c2ccc(CCc3c(Cl)cccc3Cl)cn2)cn1] | ¹H NMR (400 MHz, DMSO) δ 9.28 (s, 1H), 8.37 (d, J = 1.8 Hz, 1H), 8.10 (s, 1H), 7.91 (d, J = 2.1 Hz, 1H), 7.87 (s, 1H), 7.47 (d, J = 8.0 Hz, 2H), 7.31 (d, J = 7.6 Hz, 1H), 5.32 (s, 1H), 4.52 (s, 2H), 3.19 (dd, J = 12.0, 4.8 Hz, 2H), 2.97-2.92 (m, 2H). | 348 |
| 62 | 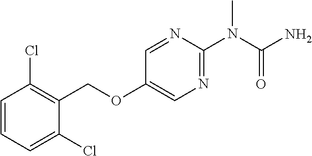 [SMILES: CN(C(N)=O)c1ncc(OCc2c(Cl)cccc2Cl)cn1] | ¹H NMR (400 MHz, DMSO) δ 8.61 (s, 2H), 7.62- 7.54 (m, 2H), 7.54-7.42 (m, 1H), 5.37 (s, 2H), 3.36 (s, 3H). | 327 |
| 63 | 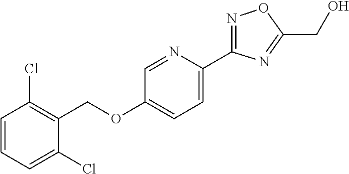 [SMILES: OCc1nc(-c2ccc(OCc3c(Cl)cccc3Cl)cn2)no1] | 1H NMR (399 MHz, DMSO) δ 8.49 (d, J = 2.9 Hz, 1H), 8.02 (d, J = 8.7 Hz, 1H), 7.72 (dd, J = 8.7, 3.0 Hz, 1H), 7.57-7.50 (m, 2H), 7.44 (dd, J = 9.0, 7.1 Hz, 1H), 6.01 (t, J = 6.4 Hz, 1H), 5.35 (s, 2H), 4.74 (d, J = 6.3 Hz, 2H). | 352 |
| 64 | 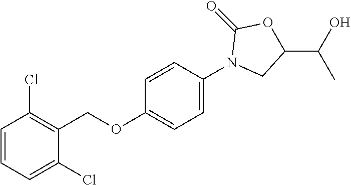 [SMILES: CC(O)C1CN(c2ccc(OCc3c(Cl)cccc3Cl)cc2)C(=O)O1] | 1H NMR (400 MHz, DMSO) δ 7.58-7.45 (m, 5H), 7.09 (d, J = 8.8 Hz, 2H), 5.22 (s, 2H), 5.13 (d, J = 5.2 Hz, 1H), 4.51-4.47 (m, 1H), 4.03 (t, J = 8.8 Hz, 1H), 3.82 (d, , J = 8.0 Hz, 1H), 3.78-3.74 (m, 1H), 1.14 (d, J = 6.4 Hz, 3H). | 382 |
| 65 | 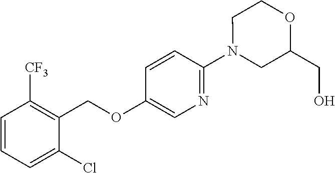 [SMILES: OCC1CN(c2ccc(OCc3c(Cl)cccc3C(F)(F)F)cn2)CCO1] | 1H NMR (399 MHz, DMSO) δ 8.03-7.97 (m, 1H), 7.93 (d, J = 8.1 Hz, 1H), 7.84 (dd, J = 8.0, 1.2 Hz, 1H), 7.69 (td, J = 8.0, 0.9 Hz, 1H), 7.42 (dd, J = 9.1, 3.1 Hz, 1H), 6.84 (d, J = 9.2 Hz, 1H), 5.20-5.15 (m, 2H), 4.78 (t, J = 5.6 Hz, 1H), 4.09- 4.01 (m, 1H), 3.98-3.84 (m, 2H), 3.63-3.37 (m, 3H), | 403 |
| | | 2.74 (td, J = 12.1, 3.5 Hz, | |
| | | 1H), 2.48-2.41 (m, 1H) | |
| 66 | 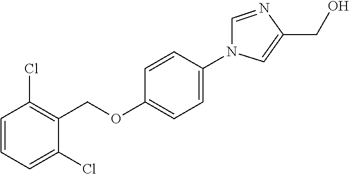 [SMILES: OCc1cn(-c2ccc(OCc3c(Cl)cccc3Cl)cc2)cn1] | 1H NMR (400 MHz, DMSO) δ 8.15 (d, J = 1.3 Hz, 1H), 7.59-7.52 (m, 3H), 7.48 (d, J = 8.0 Hz, 2H), 7.35 (d, J = 8.4 Hz, 2H), 7.33-7.27 (m, 1H), 4.40 (s, 2H), 3.15 (dd, J = 9.8, 6.7 Hz, 2H), 2.84 (dd, J = 9.4, 6.2 Hz, 2H). | 347 |
| 67 | 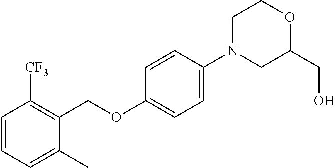 [SMILES: Cc1cccc(C(F)(F)F)c1COc1ccc(N2CCOC(CO)C2)cc1] | 1H NMR (400 MHz, DMSO) δ 7.62 (t, J = 7.6 Hz, 2H), 7.51 (t, J = 7.6 Hz, 1H), 6.97-6.89 (m, H), 5.04 (s, 2H), 4.74 (t, J = 5.6 Hz, 1H), 3.94-3.90 (m, 1H), 3.67- 3.61 (m, 1H), 3.59-3.53 (m, 1H), 3.50-3.43 (m, 2H), 3.43-3.39 (m, 1H), 3.37- 3.35 (m, 1H), 2.64-2.57 (m, 1H), 2.42 (s, 3H), 2.38-2.32 (m, 1H). | 382 |
| 68 | 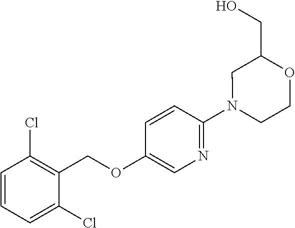 [SMILES: OCC1CN(c2ccc(OCc3c(Cl)cccc3Cl)cn2)CCO1] | 1H NMR (399 MHz, DMSO) δ 8.01-7.96 (m, 1H), 7.55 (dd, J = 8.0, 0.9 Hz, 2H), 7.50-7.38 (m, 2H), 6.82 (dd, J = 9.2, 0.7 Hz, 1H), 5.20 (s, 2H), 4.78 (t, J = 5.5 Hz, 1H), 4.04 (dt, J = 12.6, 2.0 Hz, 1H), 3.89 (dddd, J = 24.6, 12.6, 3.2, 1.5 Hz, 2H), 3.56 (td, J = 11.5, 2.7 Hz, 1H), 3.54-3.40 (m, 2H), 2.78-2.67 (m, 1H), 2.45 (dd, J = 12.5, 9.9 Hz, 1H) | 369 |
| 69 | 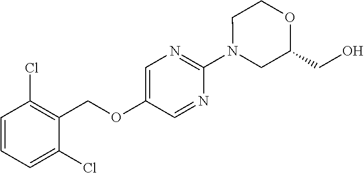 [SMILES: OC[C@@H]1CN(c2ncc(OCc3c(Cl)cccc3Cl)cn2)CCO1] | 1H NMR (399 MHz, DMSO) δ 8.32 (s, 2H), 7.56 (dd, J = 8.0, 0.9 Hz, 2H), 7.47 (dd, J = 8.9, 7.1 Hz, 1H), 5.25 (s, 2H), 4.79 (t, J = 5.7 Hz, 1H), 4.44 (dt, J = 13.2, 2.0 Hz, 1H), 4.26 (ddt, J = 13.1, 3.0, 1.5 Hz, 1H), 3.91 (ddd, J = 11.4, 3.6, 1.5 Hz, 1H), 3.55-3.34 (m, 4H), 2.90 (ddd, J = 13.2, 11.8, 3.6 Hz, 1H), 2.68-2.57 (m, 1H) | 370 |
| 70 | 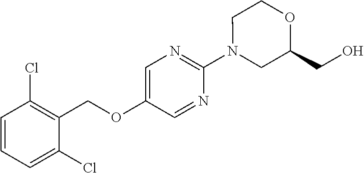 [SMILES: OC[C@H]1CN(c2ncc(OCc3c(Cl)cccc3Cl)cn2)CCO1] | 1H NMR (399 MHz, DMSO) δ 8.31 (s, 2H), 7.61-7.52 (m, 2H), 7.46 (dd, J = 8.9, 7.1 Hz, 1H), 5.25 (s, 2H), 4.79 (t, J = 5.4 Hz, 1H), 4.43 (dt, J = 13.0, 1.9 Hz, 1H), 4.26 (dp, J = 13.2, 1.4 Hz, 1H), 3.91 (ddd, J = 11.5, 3.6, 1.4 Hz, 1H), 3.55-3.36 (m, 4H), 2.89 (ddd, J = 13.1, 11.8, 3.5 Hz, 1H), 2.63 (dd, J = 13.1, 10.1 Hz, 1H) | 370 |
| 71 | 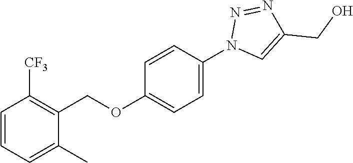 [SMILES: Cc1cccc(C(F)(F)F)c1COc1ccc(-n2cc(CO)nn2)cc1] | 1H NMR (400 MHz, DMSO) δ 8.59 (s, 1H), 7.84 (d, J = 9.0 Hz, 2H), 7.65 (t, J = 8.0 Hz, 2H), 7.55 (t, J = 7.8 Hz, 2H), 7.27 (d, J = 9.0 Hz, 2H), 5.30 (t, J = 5.6 Hz, 1H), 5.20 (s, 2H), 4.61 (d, J = 5.6 Hz, 2H), 2.44 (s, 3H). | 364 |
| 72 | 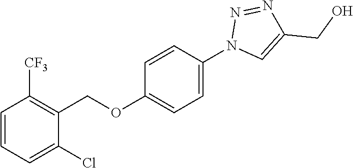 [SMILES: OCc1cn(-c2ccc(OCc3c(Cl)cccc3C(F)(F)F)cc2)nn1] | 1H NMR (400 MHz, DMSO) δ 8.60 (s, 1H), 7.95 (d, J = 8.0 Hz, 1H), 7.85 (dd, J = 12.6, 5.6 Hz, 3H), 7.71 (t, J = 8.0 Hz, 1H), 7.36-7.19 (m, 2H), 5.30 (dd, J = 10.0, 4.6 Hz, 3H), 4.61 (d, J = 5.6 Hz, 2H). | 384 |
| 73 | 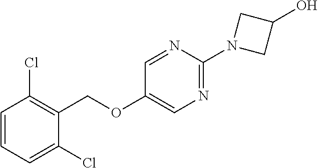 [SMILES: OC1CN(c2ncc(OCc3c(Cl)cccc3Cl)cn2)C1] | 1H NMR (400 MHz, DMSO) δ 8.26 (s, 2H), 7.56 (d, J = 8.5 Hz, 2H), 7.46 (dd, J = 8.9, 7.1 Hz, 1H), 5.62 (d, J = 6.5 Hz, 1H), 5.23 (s, 2H), 4.52 (s, 1H), 4.20-4.14 (m, 2H), 3.75-3.71 (m, 2H). | 326 |
| 74 | 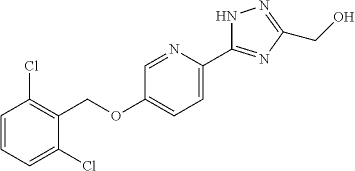 [SMILES: OCc1n[nH]c(-c2ccc(OCc3c(Cl)cccc3Cl)cn2)n1] | 1H NMR (399 MHz, MeOD) δ 8.46 (s, 1H), 8.12 (d, J = 8.6 Hz, 1H), 7.73- 7.64 (m, 1H), 7.56-7.46 (m, 2H), 7.42 (dd, J = 8.9, 7.1 Hz, 1H), 5.48 (s, 2H), 4.76 (s, 2H). | 351 |
| 75 | 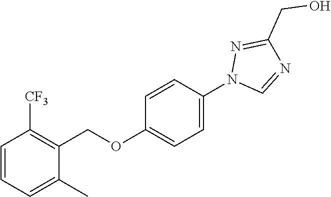 [SMILES: Cc1cccc(C(F)(F)F)c1COc1ccc(-n2cnc(CO)n2)cc1] | 1H NMR (399 MHz, DMSO) δ 9.10 (s, 1H), 7.82- 7.74 (m, 2H), 7.65 (t, J = 7.7 Hz, 2H), 7.59-7.51 (m, 1H), 7.29-7.20 (m, 2H), 5.36 (t, J = 6.0 Hz, 1H), 5.19 (d, J = 1.3 Hz, 2H), 4.52 (d, J = 6.0 Hz, 2H), 2.44 (s, 3H) | 364 |
| 76 | 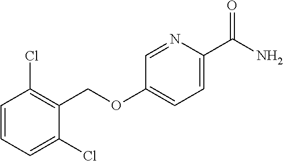 [SMILES: NC(=O)c1ccc(OCc2c(Cl)cccc2Cl)cn1] | 1H NMR (400 MHz, DMSO) δ 8.40 (d, J = 2.8 Hz, 1H), 8.04 (d, J = 8.7 Hz, 1H), 7.95 (s, 1H), 7.72 (dd, J = 8.7, 2.9 Hz, 1H), 7.63-7.56 (m, 2H), 7.50 (dd, J = 8.9, 7.1 Hz, 2H), 5.40 (s, 2H). | 297 |
| 77 | 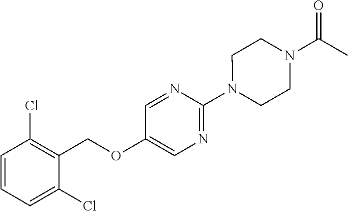 [SMILES: CC(=O)N1CCN(c2ncc(OCc3c(Cl)cccc3Cl)cn2)CC1] | 1H NMR (399 MHz, DMSO) δ 8.32 (s, 2H), 7.59- 7.53 (m, 2H), 7.47 (dd, J = 9.0, 7.1 Hz, 1H), 5.25 (s, 2H), 3.72-3.65 (m, 2H), 3.65-3.58 (m, 2H), 3.51 (dq, J = 5.5, 1.9 Hz, 4H), 2.04 (s, 3H) | 381 |
| 78 | 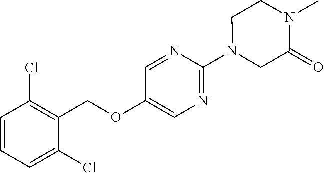 [SMILES: CN1CCN(c2ncc(OCc3c(Cl)cccc3Cl)cn2)CC1=O] | 1H NMR (399 MHz, DMSO) δ 8.36 (s, 2H), 7.59- 7.54 (m, 2H), 7.48 (dd, J = 8.9, 7.1 Hz, 1H), 5.27 (s, 2H), 4.16 (s, 2H), 3.92 (dd, J = 6.1, 4.9 Hz, 2H), 3.44- 3.37 (m, 2H), 2.89 (s, 3H) | 369 |
| 79 | 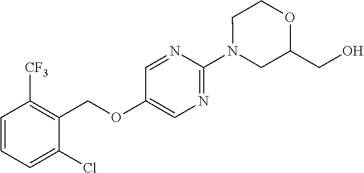 [SMILES: OCC1CN(c2ncc(OCc3c(Cl)cccc3C(F)(F)F)cn2)CCO1] | 1H NMR (399 MHz, DMSO) δ 8.34 (s, 2H), 7.94 (dd, J = 8.2, 1.2 Hz, 1H), 7.85 (dd, J = 7.9, 1.2 Hz, 1H), 7.70 (td, J = 8.0, 1.0 Hz, 1H), 5.23 (d, J = 1.2 Hz, 2H), 4.80 (t, J = 5.7 Hz, 1H), 4.45 (dt, J = 12.9, 2.1 Hz, 1H), 4.27 (ddt, J = 13.3, 3.0, 1.5 Hz, 1H), 3.92 (ddd, J = 11.4, 3.5, 1.4 Hz, 1H), 3.56-3.35 | 404 |
| | | (m, 4H), 2.91 (ddd, J = 13.2, | |
| | | 11.8, 3.6 Hz, 1H), 2.64 (dd, | |
| | | J = 13.1, 10.1 Hz, 1H) | |
| 80 | 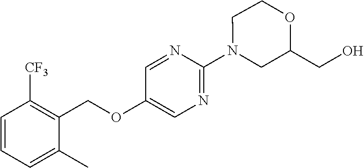 [SMILES: Cc1cccc(C(F)(F)F)c1COc1cnc(N2CCOC(CO)C2)nc1] | 1H NMR (399 MHz, DMSO) δ 8.34 (s, 2H), 7.68- 7.60 (m, 2H), 7.53 (t, J = 7.7 Hz, 1H), 5.14 (d, J = 1.3 Hz, 2H), 4.80 (t, J = 5.7 Hz, 1H), 4.45 (dt, J = 13.2, 2.0 Hz, 1H), 4.27 (ddt, J = 13.1, 3.0, 1.4 Hz, 1H), 3.92 (ddd, J = 11.4, 3.5, 1.4 Hz, 1H), 3.56- 3.35 (m, 4H), 2.91 (ddd, J = 13.2, 11.8, 3.6 Hz, 1H), | 384 |
| | | 2.64 (dd, J = 13.1, 10.1 Hz, | |
| | | 1H), 2.45 (s, 3H) | |
| 81 | 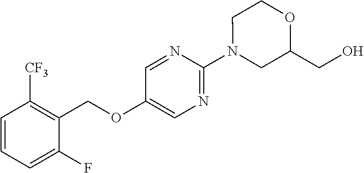 [SMILES: OCC1CN(c2ncc(OCc3c(F)cccc3C(F)(F)F)cn2)CCO1] | 1H NMR (399 MHz, DMSO) δ 8.30 (s, 2H), 7.79-7.64 (m, 3H), 5.19 (t, J = 1.4 Hz, 2H), 4.79 (t, J = 5.7 Hz, 1H), 4.44 (dt, J = 13.1, 2.0 Hz, 1H), 4.27 (ddt, J = 13.2, 3.0, 1.5 Hz, 1H), 3.92 (ddd, J = 11.4, 3.6, 1.4 Hz, 1H), 3.56-3.35 (m, 4H), 2.90 (ddd, J = 13.1, 11.8, 3.5 Hz, 1H), 2.69-2.57 (m, 1H) | 388 |
| 82 | 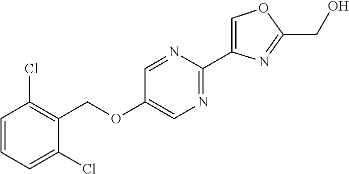 [SMILES: OCc1nc(-c2ncc(OCc3c(Cl)cccc3Cl)cn2)co1] | 1H NMR (399 MHz, DMSO) δ 8.68 (s, 2H), 8.60 (s, 1H), 7.57-7.50 (m, 2H), 7.44 (dd, J = 9.0, 7.1 Hz, 1H), 5.69 (t, J = 6.3 Hz, 1H), 5.40 (s, 2H), 4.50 (d, J = 6.2 Hz, 2H). | 352 |
| 83 | 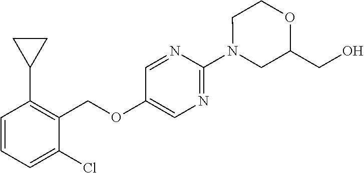 [SMILES: OCC1CN(c2ncc(OCc3c(Cl)cccc3C3CC3)cn2)CCO1] | 1H NMR (399 MHz, DMSO) δ 8.34 (s, 2H), 7.40- 7.27 (m, 2H), 7.09-6.99 (m, 1H), 5.35 (s, 2H), 4.79 (t, J = 5.7 Hz, 1H), 4.44 (dt, J = 13.0, 1.8 Hz, 1H), 4.31- 4.22 (m, 1H), 3.92 (ddd, J = 11.4, 3.6, 1.4 Hz, 1H), 3.55- 3.37 (m, 4H), 2.90 (ddd, J = 13.2, 11.8, 3.5 Hz, 1H), 2.63 (dd, J = 13.0, 10.1 Hz, 1H), | 376 |
| | | 2.13 (tt, J = 8.3, 5.3 Hz, 1H), | |
| | | 1.00-0.89 (m, 2H), 0.74- | |
| | | 0.65 (m, 2H) | |
| 84 | 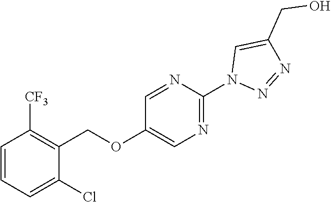 [SMILES: OCc1cn(-c2ncc(OCc3c(Cl)cccc3C(F)(F)F)cn2)nn1] | 1H NMR (399 MHz, DMSO) δ 8.91 (s, 2H), 8.61 (t, J = 0.9 Hz, 1H), 7.98 (dd, J = 8.1, 1.2 Hz, 1H), 7.89 (dd, J = 8.0, 1.2 Hz, 1H), 7.74 (td, J = 8.0, 0.9 Hz, 1H), 5.49 (d, J = 1.2 Hz, 2H), 5.33 (t, J = 5.7 Hz, 1H), 4.63 (dd, J = 5.7, 0.7 Hz, 2H) | 386 |
| 85 | 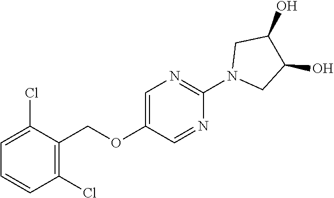 [SMILES: O[C@@H]1CN(c2ncc(OCc3c(Cl)cccc3Cl)cn2)C[C@@H]1O] | 1H NMR (399 MHz, DMSO) δ 8.25 (s, 2H), 7.56 (d, J = 7.9 Hz, 2H), 7.52- 7.42 (m, 1H), 5.22 (s, 2H), 4.89 (d, J = 4.4 Hz, 2H), 4.11 (q, J = 4.1 Hz, 2H), 3.57 (dd, J = 11.1, 5.2 Hz, 2H). | 356 |
| 86 | 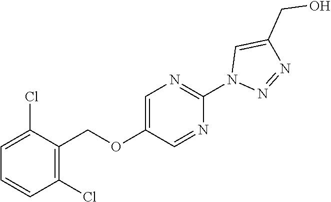 [SMILES: OCc1cn(-c2ncc(OCc3c(Cl)cccc3Cl)cn2)nn1] | 1H NMR (399 MHz, DMSO) δ 8.90 (s, 2H), 8.61 (d, J = 0.7 Hz, 1H), 7.61 (dd, J = 8.0, 0.9 Hz, 2H), 7.52 (dd, J = 9.0, 7.1 Hz, 1H), 5.53 (s, 2H), 5.33 (t, J = 5.8 Hz, 1H), 4.62 (dd, J = 5.7, 0.7 Hz, 2H) | 352 |
| 87 | 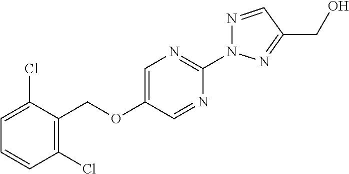 [SMILES: OCc1cnn(-c2ncc(OCc3c(Cl)cccc3Cl)cn2)n1] | 1H NMR (399 MHz, DMSO) δ 8.84 (s, 2H), 8.07 (s, 1H), 7.61 (dd, J = 8.1, 0.9 Hz, 2H), 7.52 (dd, J = 9.0, 7.1 Hz, 1H), 5.51 (s, 2H), 5.47 (t, J = 5.8 Hz, 1H), 4.65 (d, J = 5.8 Hz, 2H) | 352 |
| 88 | 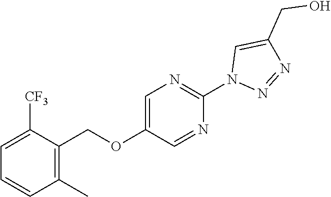 [SMILES: Cc1cccc(C(F)(F)F)c1COc1cnc(-n2cc(CO)nn2)nc1] | 1H NMR (399 MHz, DMSO) δ 8.99 (s, 2H), 8.61 (s, 1H), 7.72-7.63 (m, 2H), 7.62-7.54 (m, 1H), 5.43- 5.39 (m, 2H), 5.33 (t, J = 5.7 Hz, 1H), 4.63 (d, J = 5.5 Hz, 2H), 2.46 (s, 3H). | 366 |
| 89 | 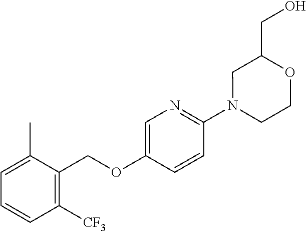 [SMILES: Cc1cccc(C(F)(F)F)c1COc1ccc(N2CCOC(CO)C2)nc1] | 1H NMR (400 MHz, DMSO) δ 8.00 (d, J = 3.2 Hz, 1H), 7.63 (t, J = 7.2 Hz, 2H), 7.52 (t, J = 8.0 Hz, 1H), 7.41 (dd, J = 9.2, 3.2 Hz, 1H), 6.84 (d, J = 9.2 Hz, 1H), 5.09 (s, 2H), 4.78-4.77 (m, 1H), 4.04 (d, J = 12.0 Hz, 1H), 3.95-3.85 (m, 2H), 3.60- 3.54 (m, 2H), 3.53-3.46 (m, 3H), 2.77-2.71 (m, 1H), 2.44 (s, 3H). | 383 |
| 90 | 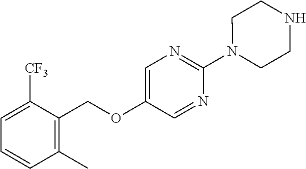 [SMILES: Cc1cccc(C(F)(F)F)c1COc1cnc(N2CCNCC2)nc1] | 1H NMR (399 MHz, DMSO) δ 8.30 (s, 2H), 7.63 (dd, J = 7.3, 5.7 Hz, 2H), 7.53 (t, J = 7.7 Hz, 1H), 5.13 (d, J = 1.2 Hz, 2H), 3.61- 3.54 (m, 4H), 2.78-2.71 (m, 4H), 2.45 (s, 3H) | 353 |
| 91 | 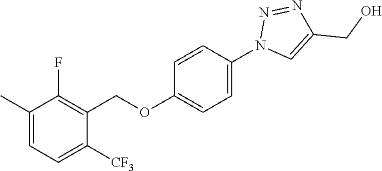 [SMILES: Cc1ccc(C(F)(F)F)c(COc2ccc(-n3cc(CO)nn3)cc2)c1F] | 1H NMR (400 MHz, DMSO) δ 8.60 (s, 1H), 7.83 (d, J = 8.8 Hz, 2H), 7.70- 7.53 (m, 2H), 7.25 (d, J = 9.0 Hz, 2H), 5.31 (s, 1H), 5.22 (s, 2H), 4.60 (s, 2H), 2.37 (s, 3H). | 382 |
| 92 | 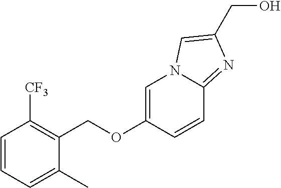 [SMILES: Cc1cccc(C(F)(F)F)c1COc1ccc2nc(CO)cn2c1] | 1H NMR (400 MHz, DMSO) δ 8.49 (d, J = 2.0 Hz, 1H), 7.73 (s, 1H), 7.65 (t, J = 7.6 Hz, 2H), 7.55 (t, J = 8.0 Hz, 1H), 7.40 (d, J = 9.6 Hz, 1H), 7.01 (dd, J = 9.6, 2.2 Hz, 1H), 5.14 (d, J = 6.0 Hz, 1H), 5.11 (s, 2H), 4.57 (d, J = 5.6 Hz, 2H), 2.45 (s, 3H). | 337 |
| 93 | 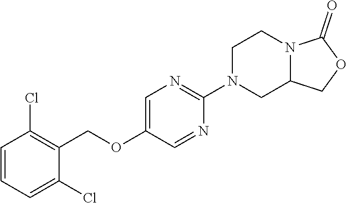 [SMILES: O=C1OCC2CN(c3ncc(OCc4c(Cl)cccc4Cl)cn3)CCN12] | 1H NMR (399 MHz, DMSO) δ 8.34 (s, 2H), 7.56 (dd, J = 8.0, 0.9 Hz, 2H), 7.47 (dd, J = 8.9, 7.1 Hz, 1H), 5.26 (s, 2H), 4.72 (ddd, J = 12.9, 4.0, 1.5 Hz, 1H), 4.57 (ddt, J = 13.1, 3.6, 1.4 Hz, 1H), 4.40 (dd, J = 8.9, 8.2 Hz, 1H), 4.01 (dd, J = 9.0, 5.6 Hz, 1H), 3.83 (dddd, J = 11.0, 8.2, 5.5, 4.0 Hz, 1H), 3.64 (ddd, J = 12.9, 3.6, 1.3 Hz, 1H), 3.00 (td, J = 12.5, | 397 |
| | | 3.7 Hz, 1H), 2.91-2.74 (m, 2H) | |
| 94 | 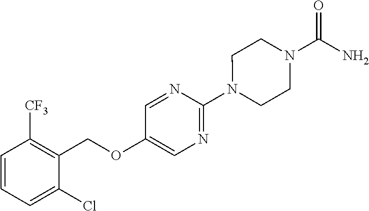 [SMILES: NC(=O)N1CCN(c2ncc(OCc3c(Cl)cccc3C(F)(F)F)cn2)CC1] | 1H NMR (399 MHz, DMSO) δ 8.33 (s, 2H), 7.67- 7.59 (m, 2H), 7.57-7.48 (m, 1H), 6.03 (s, 2H), 5.13 (d, J = 1.2 Hz, 2H), 3.64- 3.57 (m, 4H), 3.40-3.34 (m, 4H), 2.44 (s, 3H) | 396 |
| 95 | 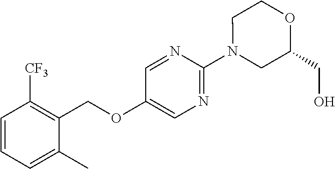 [SMILES: Cc1cccc(C(F)(F)F)c1COc1cnc(N2CCO[C@H](CO)C2)nc1] | 1H NMR (399 MHz, DMSO) δ 8.34 (s, 2H), 7.68- 7.60 (m, 2H), 7.58-7.49 (m, 1H), 5.14 (d, J = 1.3 Hz, 2H), 4.80 (t, J = 5.7 Hz, 1H), 4.45 (dt, J = 12.9, 2.0 Hz, 1H), 4.27 (dp, J = 13.1, 1.5 Hz, 1H), 3.92 (ddd, J = 11.4, 3.6, 1.4 Hz, 1H), 3.56-3.35 (m, 4H), 2.91 (ddd, J = 13.2, 11.8, 3.5 Hz, 1H), 2.64 (dd, J = | 384 |
| | | 13.0, 10.1 Hz, 1H), 2.45 (s, 3H) | |
| 96 | 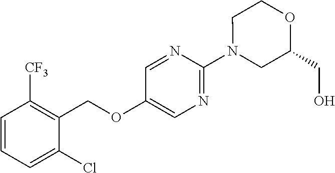 [SMILES: OC[C@@H]1CN(c2ncc(OCc3c(Cl)cccc3C(F)(F)F)cn2)CCO1] | 1H NMR (399 MHz, DMSO) δ 8.34 (s, 2H), 7.93 (dd, J = 8.1, 1.2 Hz, 1H), 7.85 (dd, J = 8.0, 1.2 Hz, 1H), 7.70 (td, J = 8.0, 0.9 Hz, 1H), 5.22 (d, J = 1.2 Hz, 2H), 4.80 (t, J = 5.6 Hz, 1H), 4.45 (dt, J = 12.8, 2.0 Hz, 1H), 4.27 (ddt, J = 13.1, 3.0, 1.5 Hz, 1H), 3.92 (ddd, J = 11.4, 3.6, 1.4 Hz, 1H), 3.56-3.35 | 404 |
| | | (m, 4H), 2.97-2.85 (m, 1H), | |
| | | 2.64 (dd, J = 13.1, 10.1 Hz, 1H) | |
| 97 | 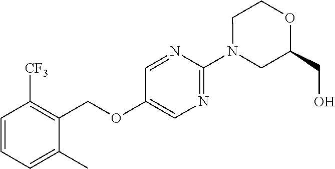 [SMILES: Cc1cccc(C(F)(F)F)c1COc1cnc(N2CCO[C@@H](CO)C2)nc1] | 1H NMR (399 MHz, DMSO) δ 8.34 (s, 2H), 7.68- 7.60 (m, 2H), 7.53 (t, J = 7.7 Hz, 1H), 5.14 (d, J = 1.3 Hz, 2H), 4.80 (t, J = 5.5 Hz, 1H), 4.45 (dt, J = 12.9, 2.0 Hz, 1H), 4.27 (ddt, J = 13.2, 3.0, 1.5 Hz, 1H), 3.92 (ddd, J = 11.4, 3.5, 1.4 Hz, 1H), 3.56-3.35 (m, 4H), 2.91 (ddd, J = 13.2, 11.8, 3.5 Hz, 1H), 2.69- 2.59 (m, 1H), 2.45 (s, 3H). | 384 |
| 98 | 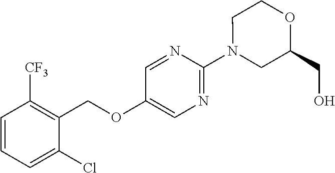 [SMILES: OC[C@H]1CN(c2ncc(OCc3c(Cl)cccc3C(F)(F)F)cn2)CCO1] | 1H NMR (399 MHz, DMSO) δ 8.34 (s, 2H), 7.93 (dd, J = 8.1, 1.2 Hz, 1H), 7.85 (dd, J = 8.0, 1.2 Hz, 1H), 7.70 (td, J = 8.0, 0.9 Hz, 1H), 5.22 (d, J = 1.2 Hz, 2H), 4.80 (t, J = 5.7 Hz, 1H), 4.45 (dt, J = 12.9, 1.9 Hz, 1H), 4.27 (dp, J = 13.1, 1.5 Hz, 1H), 3.92 (ddd, J = 11.4, 3.6 1.4 Hz, 1H), 3.56-3.35 (m, | 404 |
| | | 4H), 2.91 (ddd, J = 13.2, | |
| | | 11.8, 3.6 Hz, 1H), 2.64 (dd, | |
| | | J = 13.1, 10.1 Hz, 1H) | |
| 99 | 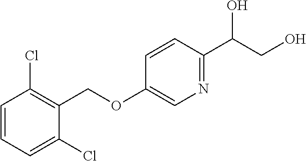 [SMILES: OCC(O)c1ccc(OCc2c(Cl)cccc2Cl)cn1] | 1H NMR (399 MHz, DMSO) δ 8.30 (dd, J = 2.9, 0.7 Hz, 1H), 7.60-7.57 (m, 2H), 7.54 (dd, J = 8.6, 3.0 Hz, 1H), 7.49 (dd, J = 9.0, 7.1 Hz, 1H), 7.44 (d, J = 8.6 Hz, 1H), 5.34-5.28 (m, 3H), 4.65 (t, J = 5.9 Hz, 1H), 4.56 (dt, J = 6.7, 4.5 Hz, 1H), 3.65 (ddd, J = 10.5, 6.0, | 314 |
| | | 4.2 Hz, 1H), 3.47 (ddd, J = | |
| | | 10.9, 6.9, 5.8 Hz, 1H). | |
| 100 | 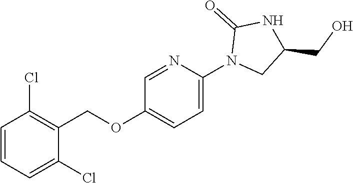 [SMILES: O=C1N[C@@H](CO)CN1c1ccc(OCc2c(Cl)cccc2Cl)cn1] | 1H NMR (399 MHz, DMSO) δ 8.16-8.09 (m, 2H), 7.59-7.56 (m, 2H), 7.52 (dd, J = 9.2, 3.1 Hz, 1H), 7.48 (dd, J = 9.0, 7.1 Hz, 1H), 7.20 (s, 1H), 5.27 (s, 2H), 4.94 (t, J = 5.5 Hz, 1H), 3.98 (dd, J = 10.6, 9.1 Hz, 1H), 3.77 (dd, J = 10.6, 5.0 Hz, 1H), 3.68 (dq, J = 10.3, 5.3 Hz, 1H), 3.46-3.35 (m, 2H) | 368 |
| 101 | 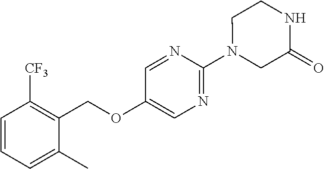 [SMILES: Cc1cccc(C(F)(F)F)c1COc1cnc(N2CCNC(=O)C2)nc1] | 1H NMR (399 MHz, DMSO) δ 8.36 (s, 2H), 8.06 (s, 1H), 7.67-7.59 (m, 2H), 7.53 (t, J = 7.7 Hz, 1H), 5.14 (d, J = 1.2 Hz, 2H), 4.10 (s, 2H), 3.84 (dd, J = 6.1, 4.7 Hz, 2H), 3.27 (td, J = 5.5, 2.6 Hz, 2H), 2.44 (s, 3H) | 367 |
| 102 | 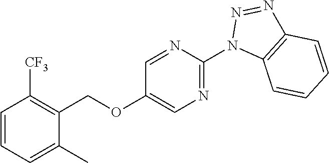 [SMILES: Cc1cccc(C(F)(F)F)c1COc1cnc(-n2nnc3ccccc32)nc1] | 1H NMR (399 MHz, DMSO) δ 9.00 (s, 2H), 8.48 (dt, J = 8.4, 1.0 Hz, 1H), 8.23 (dt, J = 8.3, 1.0 Hz, 1H), 7.76 (ddd, J = 8.3, 7.1, 1.1 Hz, 1H), 7.69 (t, J = 7.4 Hz, 2H), 7.64-7.53 (m, 2H), 5.45 (s, 2H), 3.30 (s, 1H) | 386 |
| 103 | 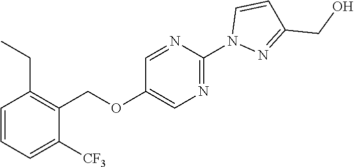 [SMILES: CCc1cccc(C(F)(F)F)c1COc1cnc(-n2ccc(CO)n2)nc1] | 1H NMR (400 MHz, DMSO) δ 8.74 (s, 2H), 8.49 (d, J = 2.4 Hz, 1H), 7.69 (d, J = 8.0 Hz, 2H), 7.64-7.60 (m, 1H), 6.53 (d, J = 2.4 Hz, 1H), 5.35 (s, 2H), 5.23 (t, J = 6.0 Hz, 1H), 4.52 (d, J = 5.6 Hz, 2H), 2.80 (q, J = 7.2 Hz, 2H), 1.22 (t, J = 7.6 Hz, 3H). | 379 |
| 104 | 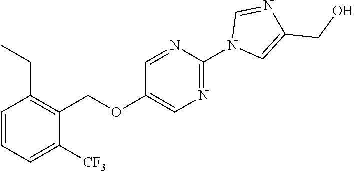 [SMILES: CCc1cccc(C(F)(F)F)c1COc1cnc(-n2cnc(CO)c2)nc1] | 1H NMR (399 MHz, DMSO) δ 8.77 (s, 2H), 8.43 (d, J = 1.4 Hz, 1H), 7.75- 7.66 (m, 3H), 7.66-7.58 (m, 1H), 5.38-5.33 (m, 2H), 5.05 (t, J = 5.7 Hz, 1H), 4.43 (dd, J = 5.7, 1.0 Hz, 2H), 2.80 (q, J = 7.5 Hz, 2H), 1.22 (t, J = 7.5 Hz, 3H). | 379 |
| 105 | 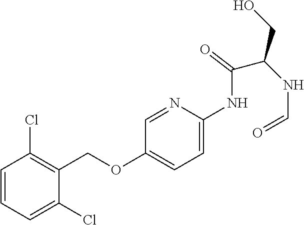 [SMILES: O=CN[C@H](CO)C(=O)Nc1ccc(OCc2c(Cl)cccc2Cl)cn1] | 1H NMR (399 MHz, DMSO) δ 10.40 (s, 1H), 8.28 (dd, J = 8.1, 1.7 Hz, 1H), 8.16 (d, J = 3.1 Hz, 1H), 8.09 (d, J = 1.5 Hz, 1H), 8.07- 8.01 (m, 1H), 7.63-7.55 (m, 3H), 7.48 (dd, J = 9.0, 7.1 Hz, 1H), 5.30 (s, 2H), 4.64 (dt, J = 8.1, 5.3 Hz, 1H), 3.72 (dd, J = 10.9, 5.2 Hz, 1H), 3.63 (dd, J = 10.9, 5.4 Hz, 1H). | 384 |
| 106 | 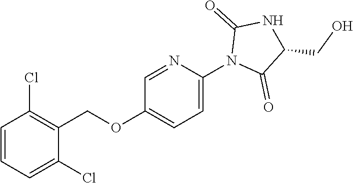 [SMILES: O=C1N[C@H](CO)C(=O)N1c1ccc(OCc2c(Cl)cccc2Cl)cn1] | 1H NMR (399 MHz, DMSO) δ 8.31 (d, J = 3.2 Hz, 2H), 7.67 (dd, J = 8.7, 3.1 Hz, 1H), 7.55-7.50 (m, 2H), 7.43 (dd, J = 8.9, 7.1 Hz, 1H), 7.25 (d, J = 8.7 Hz, 1H), 5.30 (s, 2H), 4.21-4.16 (m, 1H), 3.69 (dd, J = 11.4, 3.7 Hz, 1H), 3.61 (dd, J = 11.4, 2.9 Hz, 1H) | 382 |
| 107 | 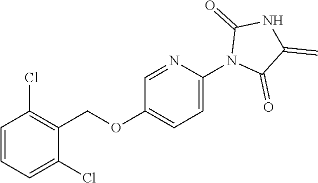 [SMILES: C=C1NC(=O)N(c2ccc(OCc3c(Cl)cccc3Cl)cn2)C1=O] | 1H NMR (399 MHz, DMSO) δ 10.84 (s, 1H), 8.41 (d, J = 3.1 Hz, 1H), 7.78 (dd, J = 8.8, 3.1 Hz, 1H), 7.63- 7.58 (m, 2H), 7.54-7.47 (m, 2H), 5.39 (s, 2H), 5.27 (d, J = 1.7 Hz, 1H), 4.96 (d, J = 1.6 Hz, 1H) | 364 |
| 108 | 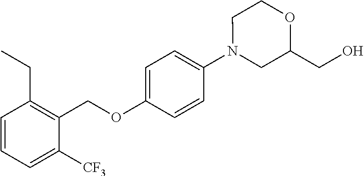 [SMILES: CCc1cccc(C(F)(F)F)c1COc1ccc(N2CCOC(CO)C2)cc1] | 1H NMR (400 MHz, CDCl3) δ 7.67-7.34 (m, 3H), 6.94 (td, J = 9.2, 2.4 Hz, 4H), 5.08 (s, 2H), 4.06 (dd, J = 11.2, 1.6 Hz, 1H), 3.96- 3.56 (m, 4H), 3.32 (t, J = 10.1 Hz, 2H), 2.93-2.71 (m, 3H), 2.71-2.52 (m, 1H), 2.04 (s, 1H), 1.28 (t, J = 7.6 Hz, 3H). | 396 |
| 109 | 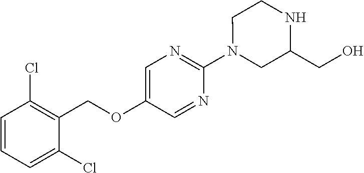 [SMILES: OCC1CN(c2ncc(OCc3c(Cl)cccc3Cl)cn2)CCN1] | 1H NMR (399 MHz, DMSO) δ 8.29 (s, 2H), 8.20 (s, 1H), 7.60-7.53 (m, 2H), 7.47 (dd, J = 8.9, 7.1 Hz, 1H), 5.25 (s, 2H), 4.47 (ddd, J = 12.6, 3.1, 1.5 Hz, 1H), 4.41-4.31 (m, 1H), 3.40 (d, J = 5.9 Hz, 2H), 3.00 (dt, J = 11.7, 2.7 Hz, 1H), 2.86 (ddd, J = 12.6, 11.5, 3.1 Hz, 1H), 2.76- 2.63 (m, 2H), 2.56 (dd, J = 12.7, | 369 |
| | | 10.4 Hz, 1H) | |
| 110 | 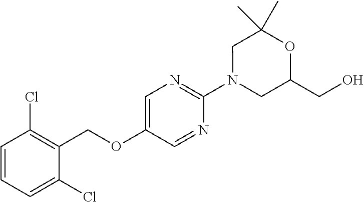 [SMILES: CC1(C)CN(c2ncc(OCc3c(Cl)cccc3Cl)cn2)CC(CO)O1] | 1H NMR (399 MHz, DMSO) δ 8.28 (s, 2H), 7.62- 7.53 (m, 2H), 7.46 (dd, J = 9.0, 7.1 Hz, 1H), 5.24 (s, 2H), 4.72 (t, J = 5.8 Hz, 1H), 4.56 (ddd, J = 12.9, 2.9, 1.6 Hz, 1H), 4.29 (dd, J = 12.8, 1.7 Hz, 1H), 3.68 (dddd, J = 11.1, 6.6, 4.8, 2.9 Hz, 1H), 3.44 (dt, J = 10.4, 5.0 Hz, 1H), 3.35 (d, J = 6.4 Hz, 0H), 2.70-2.60 (m, 1H), 1.18 (s, 3H), 1.13 (s, 3H) | 400 |
| 111 | 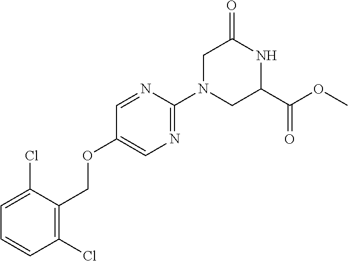 [SMILES: COC(=O)C1CN(c2ncc(OCc3c(Cl)cccc3Cl)cn2)CC(=O)N1] | 1H NMR (399 MHz, DMSO) δ 8.35 (s, 2H), 8.28 (d, J = 3.7 Hz, 1H), 7.59- 7.53 (m, 2H), 7.47 (dd, J = 9.0, 7.1 Hz, 1H), 5.26 (s, 2H), 4.64 (dd, J = 13.6, 3.2 Hz, 1H), 4.41 (dd, J = 18.1, 0.9 Hz, 1H), 4.25 (q, J = 3.7 Hz, 1H), 3.89 (d, J = 18.1 Hz, 1H), 3.69 (dd, J = 13.5, 4.3 Hz, 1H), 3.59 (s, 3H) | 411 |
| 112 | 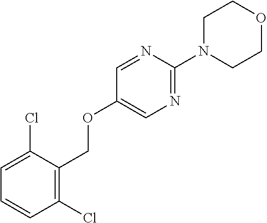 [SMILES: Clc1cccc(Cl)c1COc1cnc(N2CCOCC2)nc1] | 1H NMR (399 MHz, DMSO) δ 8.33 (s, 2H), 7.59- 7.54 (m, 2H), 7.47 (dd, J = 9.0, 7.1 Hz, 1H), 5.26 (s, 2H), 3.69-3.63 (m, 4H), 3.60 (td, J = 4.5, 1.1 Hz, 4H) | 340 |
| 113 | 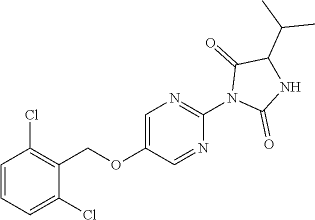 [SMILES: CC(C)C1NC(=O)N(c2ncc(OCc3c(Cl)cccc3Cl)cn2)C1=O] | 1H NMR (399 MHz, DMSO) δ 8.85 (s, 2H), 8.59 (d, J = 1.3 Hz, 1H), 7.63- 7.59 (m, 2H), 7.52 (dd, J = 9.0, 7.1 Hz, 1H), 5.50 (s, 2H), 4.26 (dd, J = 3.5, 1.3 Hz, 1H), 2.14 (pd, J = 6.9, 3.5 Hz, 1H), 1.02 (d, J = 7.0 Hz, 3H), 0.90 (d, J = 6.8 Hz, 3H) | 395 |
| 114 | 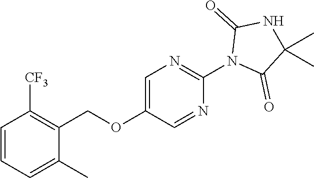 [SMILES: Cc1cccc(C(F)(F)F)c1COc1cnc(N2C(=O)NC(C)(C)C2=O)nc1] | 1H NMR (399 MHz, DMSO) δ 8.84 (s, 2H), 8.65 (s, 1H), 7.71-7.63 (m, 2H), 7.61-7.53 (m, 1H), 5.38 (s, 2H), 2.46 (s, 3H), 1.42 (s, 6H) | 395 |
| 115 | 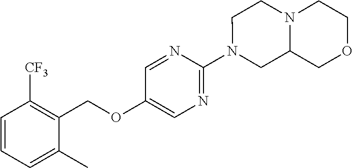 [SMILES: Cc1cccc(C(F)(F)F)c1COc1cnc(N2CCN3CCOCC3C2)nc1] | 1H NMR (400 MHz, DMSO) δ 8.31 (s, 2H), 7.64- 7.60 (m, 2H), 7.53 (t, J = 7.6 Hz, 1H), 5.13 (s, 2H), 4.44 (d, J = 12.8 Hz, 1H), 4.30 (d, J = 12.4 Hz, 1H), 3.76-3.73 (m, 2H), 3.54 (td, J = 11.6, 5.6 Hz, 1H), 3.16 (t, J = 10.4 Hz, 1H), 2.94 (td, J = 12.4, 3.2 Hz, 1H), 2.80-2.76 (m, 1H), 2.67 (d, J = 11.6 Hz, | 409 |
| | | 1H), 2.47-2.40 (m, 4H), 2.23- | |
| | | 2.05 (m, 3H). | |
| 116 | 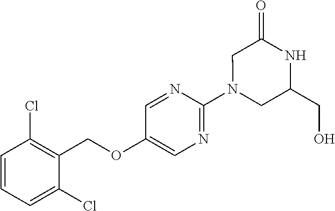 [SMILES: O=C1CN(c2ncc(OCc3c(Cl)cccc3Cl)cn2)CC(CO)N1] | 1H NMR (399 MHz, DMSO) δ 8.28 (s, 2H), 7.91 (s, 1H), 7.52-7.46 (m, 2H), 7.40 (dd, J = 8.9, 7.1 Hz, 1H), 5.19 (s, 2H), 4.87-4.80 (m, 1H), 4.07 (d, J = 18.1 Hz, 1H), 3.99-3.90 (m, 2H), 3.63 (dd, J = 13.2, 6.3 Hz, 1H), 3.42-3.20 (m, 3H) | 383 |
| 117 | 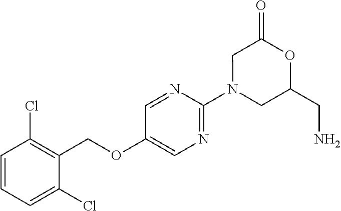 [SMILES: NCC1CN(c2ncc(OCc3c(Cl)cccc3Cl)cn2)CC(=O)O1] | 1H NMR (399 MHz, DMSO) δ 8.33 (s, 2H), 7.60- 7.54 (m, 2H), 7.48 (dd, J = 8.9, 7.1 Hz, 1H), 5.27 (s, 2H), 4.42 (dt, J = 13.2, 2.1 Hz, 1H), 4.28 (ddt, J = 13.2, 3.0, 1.5 Hz, 1H), 3.96 (ddd, J = 11.4, 3.6, 1.5 Hz, 1H), 3.57- 3.41 (m, 2H), 2.92 (ddd, J = 13.2, 11.7, 3.5 Hz, 1H), 2.78 (qd, J = 13.1, 6.0 Hz, 2H), 2.65 (dd, J = 13.1, 10.5 Hz, 1H) | 383 |
| 118 | 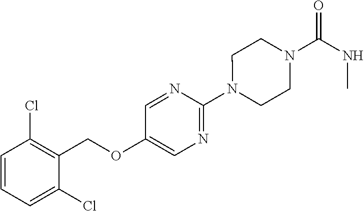 [SMILES: CNC(=O)N1CCN(c2ncc(OCc3c(Cl)cccc3Cl)cn2)CC1] | 1H NMR (399 MHz, DMSO) δ 8.32 (s, 2H), 7.57 (dd, J = 8.0, 0.9 Hz, 2H), 7.47 (dd, J = 9.0, 7.1 Hz, 1H), 6.53-6.47 (m, 1H), 5.26 (s, 2H), 3.65-3.58 (m, 4H), 3.37-3.33 (m, 4H), 2.59 (d, J = 4.3 Hz, 3H) | 396 |
| 119 | 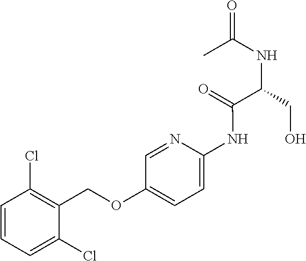 [SMILES: CC(=O)N[C@H](CO)C(=O)Nc1ccc(OCc2c(Cl)cccc2Cl)cn1] | 1H NMR (399 MHz, DMSO) δ 10.18 (s, 1H), 8.08 (d, J = 3.0 Hz, 1H), 7.96 (dd, J = 8.5, 4.0 Hz, 2H), 7.55- 7.46 (m, 3H), 7.41 (dd, J = 9.0, 7.1 Hz, 1H), 5.22 (s, 2H), 4.93 (s, 1H), 4.46 (dt, J = 7.8, 5.4 Hz, 1H), 3.67- 3.48 (m, 2H), 1.83 (s, 3H) | 398 |
| 120 | 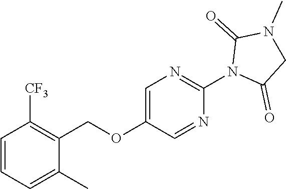 [SMILES: Cc1cccc(C(F)(F)F)c1COc1cnc(N2C(=O)CN(C)C2=O)nc1] | 1H NMR (400 MHz, DMSO) δ 8.85 (s, 2H), 7.71- 7.63 (m, 2H), 7.61-7.53 (m, 1H), 5.39 (s, 2H), 4.24 (s, 2H), 2.93 (s, 3H), 2.46 (s, 3H) | 381 |
| 121 | 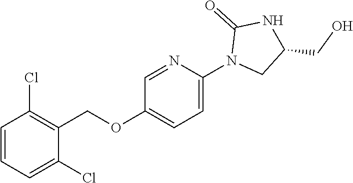 [SMILES: O=C1N[C@H](CO)CN1c1ccc(OCc2c(Cl)cccc2Cl)cn1] | 1H NMR (399 MHz, DMSO) δ 8.08-8.01 (m, 2H), 7.53-7.47 (m, 2H), 7.45 (dd, J = 9.2, 3.1 Hz, 1H), 7.40 (dd, J = 8.9, 7.1 Hz, 1H), 7.13 (s, 1H), 5.19 (s, 2H), 4.87 (s, 1H), 3.91 (dd, J = 10.6, 9.2 Hz, 1H), 3.70 (dd, J = 10.7, 5.1 Hz, 1H), 3.65-3.56 (m, 1H), 3.39-3.29 (m, 2H) | 368 |
| 122 | 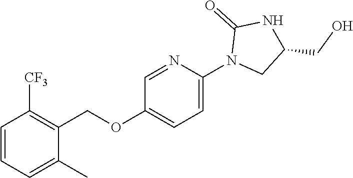 [SMILES: Cc1cccc(C(F)(F)F)c1COc1ccc(N2C[C@@H](CO)NC2=O)nc1] | 1H NMR (399 MHz, DMSO) δ 8.08-8.01 (m, 2H), 7.60-7.53 (m, 2H), 7.50-7.42 (m, 2H), 7.13 (s, 1H), 5.08 (d, J = 1.3 Hz, 2H), 4.87 (t, J = 5.5 Hz, 1H), 3.91 (dd, J = 10.6, 9.1 Hz, 1H), 3.70 (dd, J = 10.7, 5.1 Hz, 1H), 3.66-3.57 (m, 1H), 3.33 (h, J = 5.6 Hz, 2H), 2.36 (s, 3H) | 382 |
| 123 | 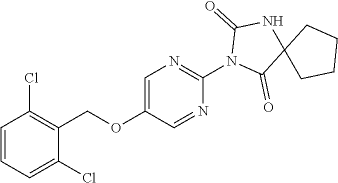 [SMILES: O=C1NC2(CCCC2)C(=O)N1c1ncc(OCc2c(Cl)cccc2Cl)cn1] | 1H NMR (399 MHz, DMSO) δ 8.85 (s, 3H), 7.63- 7.58 (m, 2H), 7.52 (dd, J = 9.0, 7.1 Hz, 1H), 5.51 (s, 2H), 2.12-2.01 (m, 2H), 1.91-1.73 (m, 6H) | 407 |
| 124 | 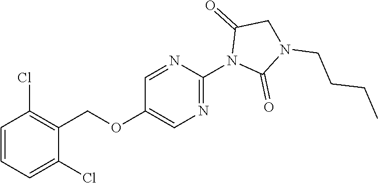 [SMILES: CCCCN1CC(=O)N(c2ncc(OCc3c(Cl)cccc3Cl)cn2)C1=O] | 1H NMR (399 MHz, DMSO) δ 8.86 (s, 2H), 7.61 (d, J = 8.5 Hz, 2H), 7.52 (dd, J = 8.9, 7.1 Hz, 1H), 5.51 (s, 2H), 4.25 (s, 2H), 3.35 (t, J = 7.2 Hz, 2H), 1.55 (p, J = 7.3 Hz, 2H), 1.32 (h, J = 7.3 Hz, 2H), 0.92 (t, J = 7.3 Hz, 2H) | 409 |
| 125 | 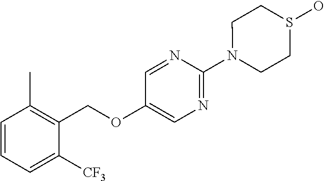 | 1H NMR (400 MHz, DMSO) δ 8.37 (d, J = 1.6 Hz, 2H), 7.64 (t, J = 6.8 Hz, 2H), 7.53 (t, J = 7.6 Hz, 1H), 5.15 (s, 2H), 4.33 (d, J = 14.2 Hz, 2H), 4.05-3.82 (m, 2H), 2.87 (t, J = 11.0 Hz, 2H), 2.69 (d, J = 12.0 Hz, 2H), 2.45 (s, 3H). | 386 |
| 126 | 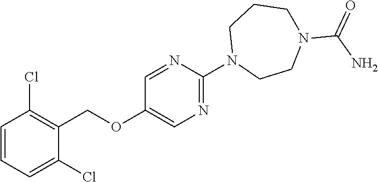 [SMILES: NC(=O)N1CCCN(c2ncc(OCc3c(Cl)cccc3Cl)cn2)CC1] | 1H NMR (399 MHz, DMSO) δ 8.26 (s, 2H), 7.59- 7.52 (m, 2H), 7.46 (dd, J = 8.9, 7.1 Hz, 1H), 5.80 (s, 2H), 5.22 (s, 2H), 3.74 (dd, J = 6.4, 4.4 Hz, 2H), 3.66 (t, J = 6.0 Hz, 2H), 3.48-3.41 (m, 2H), 3.26 (t, J = 6.0 Hz, 2H), 1.78 (p, J = 6.1 Hz, 2H) | 396 |
| 127 | 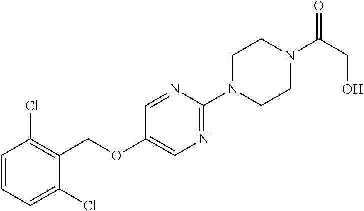 [SMILES: O=C(CO)N1CCN(c2ncc(OCc3c(Cl)cccc3Cl)cn2)CC1] | 1H NMR (399 MHz, DMSO) δ 8.33 (s, 2H), 7.59- 7.53 (m, 2H), 7.47 (dd, J = 8.9, 7.1 Hz, 1H), 5.26 (s, 2H), 4.61 (t, J = 5.5 Hz, 1H), 4.13 (d, J = 5.5 Hz, 2H), 3.66 (q, J = 5.1 Hz, 4H), 3.55 (d, J = 5.5 Hz, 2H), 3.42 (t, J = 5.2 Hz, 2H) | 397 |
| 128 | 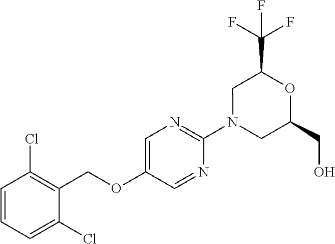 [SMILES: OC[C@H]1CN(c2ncc(OCc3c(Cl)cccc3Cl)cn2)C[C@@H](C(F)(F)F)O1] | 1H NMR (399 MHz, DMSO) δ 8.38 (s, 2H), 7.57 (d, J = 1.2 Hz, 1H), 7.55 (s, 1H), 7.47 (dd, J = 9.0, 7.1 Hz, 1H), 5.27 (s, 2H), 4.95 (t, J = 5.7 Hz, 1H), 4.57 (dddd, J = 20.4, 13.3, 2.8, 1.4 Hz, 2H), 4.32 (dtd, J = 11.0, 6.7, 2.8 Hz, 1H), 3.71-3.62 (m, 1H), 3.56 (dt, J = 10.7, 5.2 Hz, 1H), 3.48 (dt, J = 11.6, 5.9 Hz, 1H), 2.90 (dd, J = 12.8, 11.0 Hz, 1H), 2.71 (dd, J = 13.3, 10.9 Hz, 1H) | 438 |
| 129 | 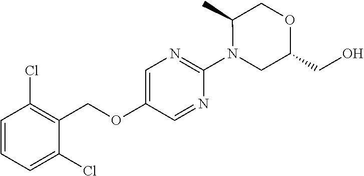 [SMILES: C[C@H]1CO[C@H](CO)CN1c1ncc(OCc2c(Cl)cccc2Cl)cn1] | 1H NMR (399 MHz, DMSO) δ 8.32 (s, 2H), 7.57 (dd, J = 8.1, 0.8 Hz, 2H), 7.47 (dd, J = 8.9, 7.1 Hz, 1H), 5.25 (s, 2H), 4.67 (dd, J = 6.2, 4.8 Hz, 1H), 4.38 (dt, J = 6.8, 3.3 Hz, 1H), 4.07 (dd, J = 13.6, 2.8 Hz, 1H), 3.87 (dd, J = 11.7, 3.9 Hz, 1H), 3.84-3.76 (m, 1H), 3.59-3.37 (m, 4H), 1.17 (d, J = 6.5 Hz, 3H) | 384 |
| 130 | 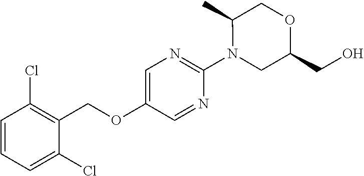 [SMILES: C[C@H]1CO[C@@H](CO)CN1c1ncc(OCc2c(Cl)cccc2Cl)cn1] | 1H NMR (399 MHz, DMSO) δ 8.32 (s, 2H), 7.57 (dd, J = 8.0, 0.9 Hz, 2H), 7.47 (dd, J = 8.9, 7.1 Hz, 1H), 5.25 (s, 2H), 4.81 (t, J = 5.7 Hz, 1H), 4.54-4.44 (m, 1H), 4.27 (dd, J = 13.4, 2.7 Hz, 1H), 3.74 (dd, J = 11.4, 1.1 Hz, 1H), 3.64 (dd, J = 11.4, 3.2 Hz, 1H), 3.58- 3.36 (m, 3H), 2.77 (dd, J = | 384 |
| | | 13.3, 10.7 Hz, 1H), 1.12 (d, | |
| | | J = 6.7 Hz, 3H) | |
| 131 | 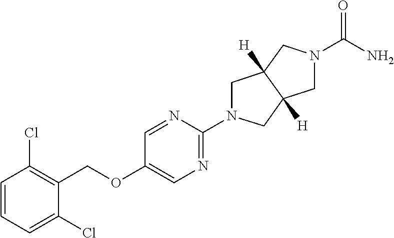 [SMILES: NC(=O)N1C[C@@H]2CN(c3ncc(OCc4c(Cl)cccc4Cl)cn3)C[C@@H]2C1] | 1H NMR (399 MHz, DMSO) δ 8.27 (s, 2H), 7.59- 7.52 (m, 2H), 7.46 (dd, J = 8.9, 7.1 Hz, 1H), 5.71 (s, 2H), 5.22 (s, 2H), 3.68 (dd, J = 11.3, 7.3 Hz, 2H), 3.49 (dd, J = 10.6, 7.3 Hz, 2H), 3.35-3.27 (m, 2H), 3.14 (dd, J = 10.7, 3.9 Hz, 2H), 3.02-2.89 (m, 2H) | 408 |
| 132 | 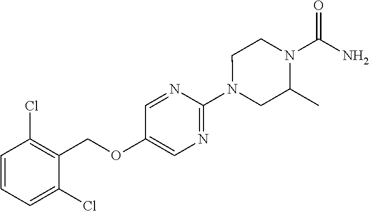 [SMILES: CC1CN(c2ncc(OCc3c(Cl)cccc3Cl)cn2)CCN1C(N)=O] | 1H NMR (399 MHz, DMSO) δ 8.29 (s, 2H), 7.56 (d, J = 8.6 Hz, 2H), 7.46 (dd, J = 9.0, 7.1 Hz, 1H), 5.98 (s, 2H), 5.24 (s, 2H), 4.42-4.33 (m, 1H), 4.30 (dt, J = 12.9, 2.0 Hz, 1H), 4.21 (ddd, J = 10.6, 5.2, 3.2 Hz, 1H), 3.77 (dt, J = 12.9, 3.0 Hz, 1H), 3.13-2.93 (m, 2H), 2.86 (td, J = 12.3, 3.5 Hz, 1H), 1.02 (d, J = 6.6 Hz, 3H) | 396 |
| 133 | 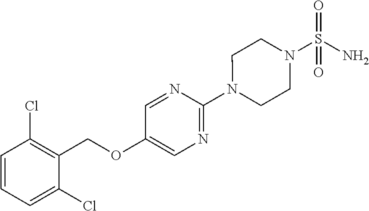 [SMILES: NS(=O)(=O)N1CCN(c2ncc(OCc3c(Cl)cccc3Cl)cn2)CC1] | 1H NMR (399 MHz, DMSO) δ 8.33 (s, 2H), 7.61- 7.52 (m, 2H), 7.47 (dd, J = 8.9, 7.1 Hz, 1H), 6.81 (s, 2H), 5.26 (s, 2H), 3.79-3.72 (m, 4H), 3.04-2.97 (m, 4H) | 418 |
| 134 | 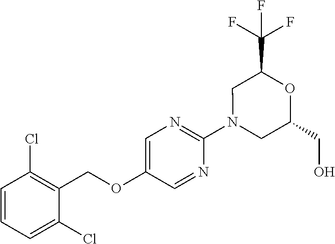 [SMILES: OC[C@@H]1CN(c2ncc(OCc3c(Cl)cccc3Cl)cn2)C[C@@H](C(F)(F)F)O1] | 1H NMR (399 MHz, DMSO) δ 8.36 (s, 2H), 7.64- 7.54 (m, 2H), 7.48 (dd, J = 9.0, 7.1 Hz, 1H), 5.27 (s, 2H), 4.87 (t, J = 5.7 Hz, 1H), 4.51 (dtd, J = 13.2, 8.2, 4.9 Hz, 1H), 4.09-4.00 (m, 2H), 3.97-3.87 (m, 2H), 3.83 (dd, J = 13.8, 4.5 Hz, 1H), 3.59-3.36 (m, 3H) | 438 |
| 135 | 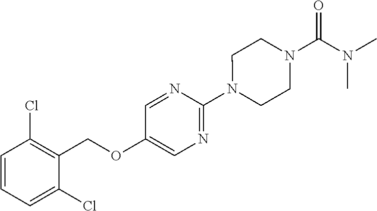 [SMILES: CN(C)C(=O)N1CCN(c2ncc(OCc3c(Cl)cccc3Cl)cn2)CC1] | 1H NMR (399 MHz, DMSO) δ 8.32 (s, 2H), 7.57 (dd, J = 8.1, 0.9 Hz, 2H), 7.47 (dd, J = 8.9, 7.1 Hz, 1H), 5.26 (s, 2H), 3.70-3.62 (m, 4H), 3.21-3.14 (m, 4H), 2.78 (s, 6H) | 410 |
| 136 | 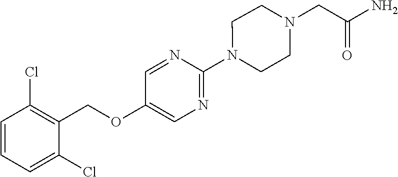 [SMILES: NC(=O)CN1CCN(c2ncc(OCc3c(Cl)cccc3Cl)cn2)CC1] | 1H NMR (399 MHz, DMSO) δ 8.29 (s, 2H), 7.57 (d, J = 1.2 Hz, 1H), 7.55 (s, 1H), 7.46 (dd, J = 9.0, 7.1 Hz, 1H), 7.24 (s, 1H), 7.14 (s, 1H), 5.24 (s, 2H), 3.71- 3.64 (m, 4H), 2.89 (s, 2H), 2.47 (m, 4H) | 396 |
| 137 | 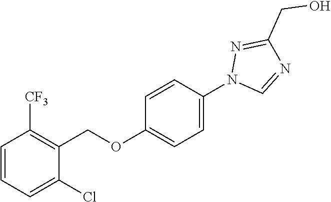 [SMILES: OCc1ncn(-c2ccc(OCc3c(Cl)cccc3C(F)(F)F)cc2)n1] | 1H NMR (399 MHz, DMSO) δ 9.16 (s, 1H), 8.01 (dd, J = 8.1, 1.2 Hz, 1H), 7.92 (dd, J = 8.1, 1.2 Hz, 1H), 7.88-7.80 (m, 2H), 7.77 (td, J = 8.0, 0.9 Hz, 1H), 7.35-7.26 (m, 2H), 5.42 (t, J = 6.0 Hz, 1H), 5.32 (d, J = 1.2 Hz, 2H), 4.58 (d, J = 5.9 Hz, 2H) | 384 |
| 138 | 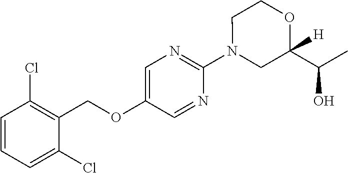 [SMILES: C[C@@H](O)[C@@H]1CN(c2ncc(OCc3c(Cl)cccc3Cl)cn2)CCO1] | 1H NMR (399 MHz, DMSO) δ 8.31 (s, 2H), 7.56 (dd, J = 8.0, 0.9 Hz, 2H), 7.47 (dd, J = 8.9, 7.1 Hz, 1H), 5.25 (s, 2H), 4.79 (d, J = 5.7 Hz, 1H), 4.60 (dt, J = 13.2, 2.4 Hz, 1H), 4.26 (ddt, J = 13.0, 2.9, 1.4 Hz, 1H), 3.92 (ddd, J = 11.4, 3.6, 1.4 Hz, 1H), 3.58-3.42 (m, 2H), 3.09 (ddd, J = 10.0, 7.1, | 384 |
| | | 2.6 Hz, 1H), 2.87 (ddd, J = | |
| | | 13.1, 11.8, 3.5 Hz, 1H), 2.62 | |
| | | (dd, J = 13.2, 10.5 Hz, 1H), | |
| | | 1.13 (d, J = 6.2 Hz, 3H) | |
| 139 | 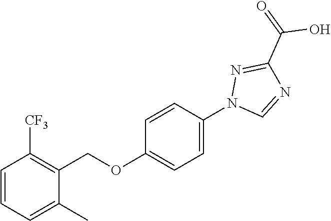 [SMILES: Cc1cccc(C(F)(F)F)c1COc1ccc(-n2cnc(C(=O)O)n2)cc1] | 1H NMR (399 MHz, DMSO) δ 13.41 (s, 1H), 9.23 (s, 1H), 7.81-7.73 (m, 2H), 7.59 (t, J = 7.6 Hz, 2H), 7.48 (t, J = 7.8 Hz, 1H), 7.25- 7.17 (m, 2H), 5.14 (s, 2H), 2.37 (s, 3H) | 378 |
| 140 | 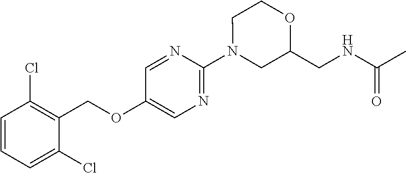 [SMILES: CC(=O)NCC1CN(c2ncc(OCc3c(Cl)cccc3Cl)cn2)CCO1] | 1H NMR (399 MHz, DMSO) δ 8.32 (s, 2H), 8.03 (t, J = 5.9 Hz, 1H), 7.60-7.53 (m, 2H), 7.47 (dd, J = 8.9, 7.1 Hz, 1H), 5.26 (s, 2H), 4.39-4.30 (m, 1H), 4.25 (ddt, J = 13.3, 3.0, 1.5 Hz, 1H), 3.92 (ddd, J = 11.5, 3.6, 1.4 Hz, 1H), 3.54-3.38 (m, 2H), 3.26- 3.06 (m, 2H), 2.90 (ddd, J = 13.1, 11.7, 3.5 Hz, 1H), 2.60 (dd, J = 13.1, 10.5 Hz, 1H), 1.83 (s, | 411 |
| | | 3H) | |
| 141 | 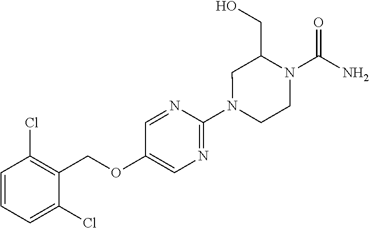 [SMILES: NC(=O)N1CCN(c2ncc(OCc3c(Cl)cccc3Cl)cn2)CC1CO] | 1H NMR (399 MHz, DMSO) δ 8.30 (s, 2H), 7.59-7.53 (m, 2H), 7.47 (dd, J = 8.9, 7.1 Hz, 1H), 5.94 (s, 2H), 5.25 (s, 2H), 4.71 (dd, J = 5.9, 4.6 Hz, 1H), 4.50 (ddd, J = 13.1, 2.5, 1.4 Hz, 1H), 4.23 (ddd, J = 11.7, 3.6, 1.9 Hz, 1H), 4.03 (dt, J = 11.8, 4.5 Hz, 1H), 3.89-3.78 (m, 1H), 3.47-3.32 (m, 2H), 3.08-2.88 (m, 3H) | 414 |
| 142 | 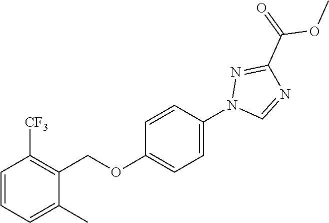 [SMILES: COC(=O)c1ncn(-c2ccc(OCc3c(C)cccc3C(F)(F)F)cc2)n1] | 1H NMR (399 MHz, DMSO) δ 9.43 (s, 1H), 7.96- 7.88 (m, 2H), 7.72 (t, J = 7.6 Hz, 2H), 7.61 (t, J = 7.7 Hz, 1H), 7.39-7.30 (m, 2H), 5.27 (d, J = 1.3 Hz, 2H), 3.97 (s, 3H), 3.36 (s, 1H), 2.50 (s, 3H) | 392 |
| 143 | 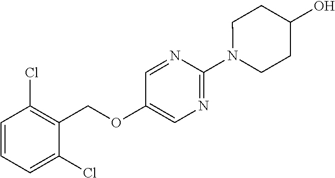 [SMILES: OC1CCN(c2ncc(OCc3c(Cl)cccc3Cl)cn2)CC1] | 1H NMR (399 MHz, DMSO) δ 8.19 (s, 2H), 7.53- 7.46 (m, 2H), 7.40 (dd, J = 8.9, 7.1 Hz, 1H), 5.16 (s, 2H), 4.62 (d, J = 4.3 Hz, 1H), 4.16-4.05 (m, 2H), 3.63 (dq, J = 8.9, 4.4 Hz, 1H), 3.11 (ddd, J = 13.3, 10.0, 3.2 Hz, 2H), 1.73-1.62 (m, 2H), 1.30-1.18 (m, 2H). | 354 |
| 144 | 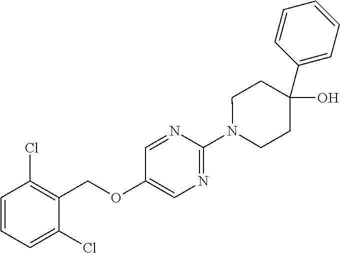 [SMILES: OC1(c2ccccc2)CCN(c2ncc(OCc3c(Cl)cccc3Cl)cn2)CC1] | 1H NMR (399 MHz, DMSO) δ 8.29 (s, 2H), 7.59- 7.55 (m, 2H), 7.51-7.44 (m, 3H), 7.32 (dd, J = 8.4, 6.9 Hz, 2H), 7.25-7.18 (m, 1H), 5.25 (s, 2H), 5.10 (s, 1H), 4.51-4.42 (m, 2H), 3.37-3.23 (m, 2H), 1.88 (td, J = 13.0, 4.6 Hz, 2H), 1.70- 1.61 (m, 2H). | 430 |
| 145 | 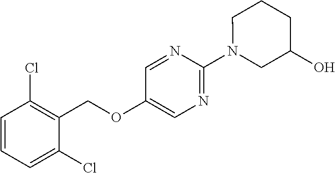 [SMILES: OC1CCCN(c2ncc(OCc3c(Cl)cccc3Cl)cn2)C1] | 1H NMR (399 MHz, DMSO) δ 8.53 (s, 1H), 8.26 (s, 2H), 7.59-7.54 (m, 2H), 7.47 (dd, J = 8.9, 7.1 Hz, 1H), 5.23 (s, 2H), 4.88 (s, 1H), 4.43-4.32 (m, 1H), 4.22 (dt, J = 13.4, 4.0 Hz, 1H), 3.49-3.39 (m, 1H), 2.99-2.88 (m, 1H), 2.79 (dd, J = 12.5, 9.1 Hz, 1H), 1.94-1.85 (m, 1H), 1.77-1.64 | 354 |
| | | (m, 1H), 1.43-1.30 (m, 2H). | |
| 146 | 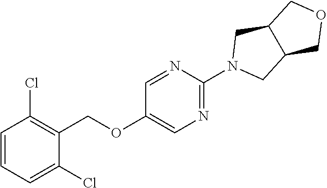 [SMILES: Clc1cccc(Cl)c1COc1cnc(N2C[C@H]3COC[C@H]3C2)nc1] | 1H NMR (399 MHz, DMSO) δ 8.27 (s, 2H), 7.59- 7.53 (m, 2H), 7.47 (dd, J = 8.9, 7.1 Hz, 1H), 5.24 (s, 2H), 3.83 (dd, J = 8.8, 6.6 Hz, 2H), 3.67-3.60 (m, 2H), 3.55 (dd, J = 8.8, 3.5 Hz, 2H), 3.40 (dd, J = 11.4, 3.2 Hz, 2H), 3.06-2.94 (m, 2H). | 366 |
| 147 | 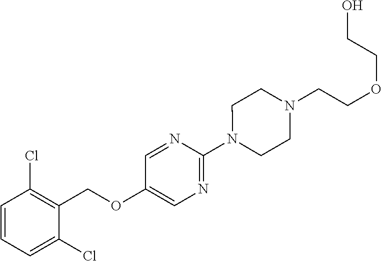 [SMILES: OCCOCCN1CCN(c2ncc(OCc3c(Cl)cccc3Cl)cn2)CC1] | 1H NMR (399 MHz, DMSO) δ 8.29 (s, 2H), 7.59- 7.54 (m, 2H), 7.47 (dd, J = 8.9, 7.1 Hz, 1H), 5.25 (s, 2H), 3.66-3.60 (m, 4H), 3.55 (t, J = 5.9 Hz, 2H), 3.53- 3.47 (m, 2H), 3.45-3.40 (m, 2H), 2.50-2.44 (m, 4H). | 427 |
| 148 | 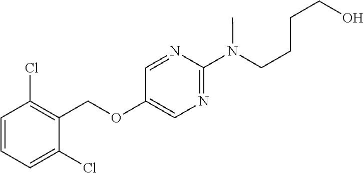 [SMILES: CN(CCCCO)c1ncc(OCc2c(Cl)cccc2Cl)cn1] | 1H NMR (399 MHz, DMSO) δ 8.17 (s, 2H), 7.52- 7.46 (m, 2H), 7.39 (dd, J = 8.9, 7.1 Hz, 1H), 5.15 (s, 2H), 4.31 (t, J = 5.2 Hz, 1H), 3.48 (t, J = 7.3 Hz, 2H), 3.33 (td, J = 6.4, 5.1 Hz, 2H), 2.97 (s, 3H), 1.54- 1.43 (m, 2H), 1.39-1.27 (m, 2H). | 356 |
| 149 | 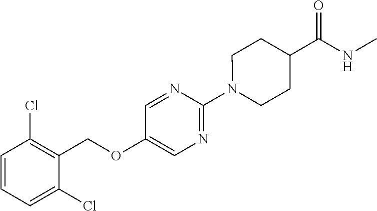 [SMILES: CNC(=O)C1CCN(c2ncc(OCc3c(Cl)cccc3Cl)cn2)CC1] | 1H NMR (399 MHz, DMSO) δ 8.28 (s, 2H), 7.75 (q, J = 4.5 Hz, 1H), 7.60 7.53 (m, 2H), 7.47 (dd, J = 8.9, 7.1 Hz, 1H), 5.24 (s, 2H), 4.54 (dt, J = 13.7, 3.5 Hz, 2H), 2.93-2.79 (m, 2H), 2.56 (d, J = 4.5 Hz, 3H), 2.36 (ddt, J = 11.6, 7.8, 3.9 Hz, 1H), 1.75-1.63 (m, 2H), 1.54-1.39 (m, 2H). | 395 |
| 150 | 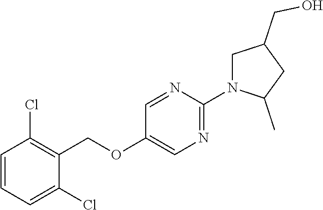 [SMILES: CC1CC(CO)CN1c1ncc(OCc2c(Cl)cccc2Cl)cn1] | 1H NMR (399 MHz, DMSO) δ 8.28-8.24 (m, 2H), 7.59-7.54 (m, 2H), 7.47 (dd, J = 8.9, 7.1 Hz, 1H), 5.25-5.19 (m, 2H), 4.67 (q, J = 5.1 Hz, 1H), 4.21-3.96 (m, 1H), 3.87- 3.54 (m, 1H), 3.52-3.36 (m, 2H), 3.22-3.13 (m, 1H), 2.61-1.37 (m, 3H), 1.29-1.14 (m, 3H). | 368 |
| 151 | 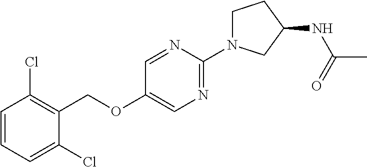 [SMILES: CC(=O)N[C@@H]1CCN(c2ncc(OCc3c(Cl)cccc3Cl)cn2)C1] | 1H NMR (399 MHz, DMSO) δ 8.27 (s, 2H), 8.13 (d, J = 6.7 Hz, 1H), 7.59- 7.53 (m, 2H), 7.47 (dd, J = 8.9, 7.1 Hz, 1H), 5.23 (s, 2H), 4.36-4.26 (m, 1H), 3.63 (dd, J = 11.3, 6.3 Hz, 1H), 3.59-3.44 (m, 2H), 3.38-3.22 (m, 1H), 2.19- 2.07 (m, 1H), 1.90-1.77 (m, 4H). | 381 |
| 152 | 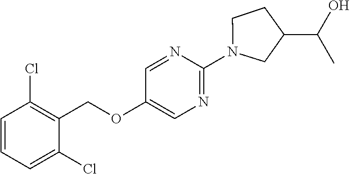 [SMILES: CC(O)C1CCN(c2ncc(OCc3c(Cl)cccc3Cl)cn2)C1] | 1H NMR (399 MHz, DMSO) δ 8.24 (d, J = 0.9 Hz, 2H), 7.58-7.53 (m, 2H), 7.46 (dd, J = 9.0, 7.1 Hz, 1H), 5.22 (s, 2H), 4.71-4.05 (m, 1H), 3.69-3.48 (m, 3H), 3.40-3.27 (m, 1H), 3.27- 3.04 (m, 2H), 2.24-2.10 (m, 1H), 2.10-1.84 (m, 1H), 1.83-1.60 (m, 1H), 1.15- 1.06 (m, 3H). | 368 |
| 153 | 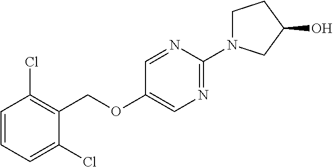 [SMILES: O[C@@H]1CCN(c2ncc(OCc3c(Cl)cccc3Cl)cn2)C1] | 1H NMR (399 MHz, DMSO) δ 8.25 (s, 2H), 7.56 (dd, J = 8.0, 0.9 Hz, 2H), 7.46 (dd, J = 8.9, 7.1 Hz, 1H), 5.23 (s, 2H), 4.93 (d, J = 3.5 Hz, 1H), 4.36 (dt, J = 6.3, 2.6 Hz, 1H), 3.57-3.43 (m, 3H), 3.42-3.35 (m, 1H), 2.00 (dtd, J = 13.2, 8.7, 4.6 Hz, 1H), 1.91-1.82 (m, 1H). | 340 |
| 154 | 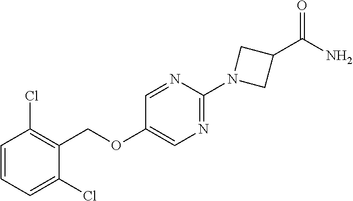 [SMILES: NC(=O)C1CN(c2ncc(OCc3c(Cl)cccc3Cl)cn2)C1] | 1H NMR (399 MHz, DMSO) δ 8.21 (s, 2H), 7.53- 7.44 (m, 2H), 7.44-7.35 (m, 2H), 6.94 (s, 1H), 5.17 (s, 2H), 4.01 (t, J = 8.4 Hz, 2H), 3.91 (dd, J = 8.3, 6.0 Hz, 2H), 3.32 (ddd, J = 8.6, 6.0, 2.5 Hz, 1H). | 353 |
| 155 | 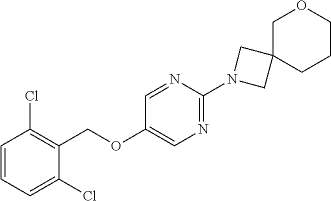 [SMILES: Clc1cccc(Cl)c1COc1cnc(N2CC3(CCCOC3)C2)nc1] | 1H NMR (399 MHz, DMSO) δ 8.27 (s, 2H), 7.59- 7.53 (m, 2H), 7.47 (dd, J = 9.0, 7.1 Hz, 1H), 5.24 (s, 2H), 3.70 (d, J = 8.4 Hz, 2H), 3.67-3.59 (m, 4H), 3.56- 3.47 (m, 2H), 1.83-1.75 (m, 2H), 1.57-1.45 (m, 2H). | 380 |
| 156 | 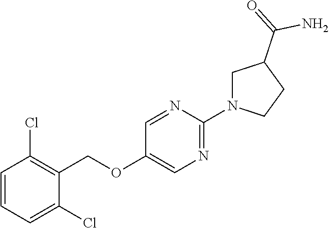 [SMILES: NC(=O)C1CCN(c2ncc(OCc3c(Cl)cccc3Cl)cn2)C1] | 1H NMR (399 MHz, DMSO) δ 8.26 (s, 2H), 7.59- 7.52 (m, 2H), 7.46 (dd, J = 8.9, 7.1 Hz, 2H), 6.93 (s, 1H), 5.23 (s, 2H), 3.66 (dd, J = 10.8, 7.9 Hz, 1H), 3.57 (ddd, J = 10.6, 7.9, 4.7 Hz, 1H), 3.52-3.37 (m, 2H), 3.01 (p, J = 7.5 Hz, 1H), 2.20- 1.99 (m, 2H). | 367 |
| 157 | 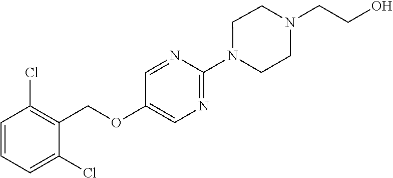 [SMILES: OCCN1CCN(c2ncc(OCc3c(Cl)cccc3Cl)cn2)CC1] | 1H NMR (399 MHz, DMSO) δ 8.29 (s, 2H), 7.60- 7.53 (m, 2H), 7.47 (dd, J = 8.9, 7.1 Hz, 1H), 5.25 (s, 2H), 3.67-3.58 (m, 4H), 3.53 (t, J = 6.2 Hz, 2H), 2.49- 2.44 (m, 4H), 2.42 (t, J = 6.3 Hz, 2H). | 383 |
| 158 | 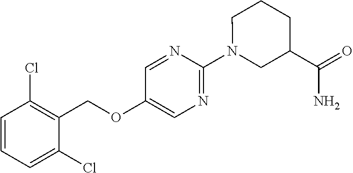 [SMILES: NC(=O)C1CCCN(c2ncc(OCc3c(Cl)cccc3Cl)cn2)C1] | 1H NMR (399 MHz, DMSO) δ 8.21 (s, 2H), 7.53- 7.45 (m, 2H), 7.40 (dd, J = 8.9, 7.1 Hz, 1H), 7.28-7.21 (m, 1H), 6.80-6.68 (m, 1H), 5.17 (s, 2H), 4.55-4.36 (m, 2H), 2.85-2.66 (m, 2H), 2.24-2.12 (m, 1H), 1.89- 1.75 (m, 1H), 1.66-1.46 (m, 2H), 1.39-1.24 (m, 1H). | 381 |
| 159 | 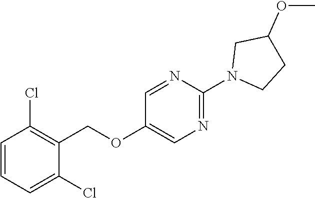 [SMILES: COC1CCN(c2ncc(OCc3c(Cl)cccc3Cl)cn2)C1] | 1H NMR (399 MHz, DMSO) δ 8.26 (s, 2H), 7.56 (dd, J = 8.0, 0.9 Hz, 2H), 7.46 (dd, J = 9.0, 7.1 Hz, 1H), 5.23 (s, 2H), 4.05 (qd, J = 4.1, 2.7 Hz, 1H), 3.60- 3.48 (m, 3H), 3.46-3.35 (m, 1H), 3.26 (s, 3H), 2.03 (ddd, J = 7.9, 6.7, 4.3 Hz, 2H). | 354 |
| 160 | 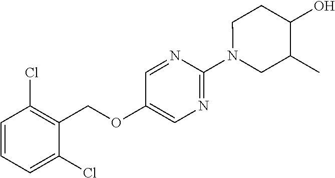 [SMILES: CC1CN(c2ncc(OCc3c(Cl)cccc3Cl)cn2)CCC1O] | 1H NMR (399 MHz, DMSO) δ 8.26 (s, 2H), 7.60- 7.51 (m, 2H), 7.47 (dd, J = 9.0, 7.1 Hz, 1H), 5.23 (s, 2H), 4.66 (d, J = 5.4 Hz, 1H), 4.43 (dddd, J = 24.2, 13.1, 4.5, 2.3 Hz, 2H), 3.17 (d, J = 3.3 Hz, 2H), 2.90 (ddd, J = 13.3, 12.2, 2.8 Hz, 1H), 1.87- 1.71 (m, 1H), 1.39-1.20 (m, 2H), 0.94 (d, J = 6.5 Hz, 3H). | 368 |
| 161 | 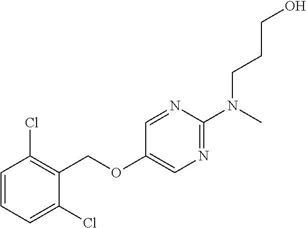 [SMILES: CN(CCCO)c1ncc(OCc2c(Cl)cccc2Cl)cn1] | 1H NMR (399 MHz, DMSO) δ 8.25 (s, 2H), 7.58- 7.53 (m, 2H), 7.47 (dd, J = 8.9, 7.1 Hz, 1H), 5.22 (s, 2H), 4.47 (t, J = 4.9 Hz, 1H), 3.63-3.55 (m, 2H), 3.43 (q, J = 5.9 Hz, 2H), 3.05 (s, 3H), 1.75-1.64 (m, 2H). | 342 |
| 162 | 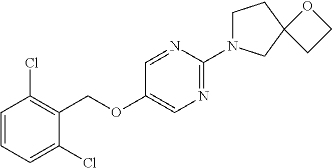 [SMILES: Clc1cccc(Cl)c1COc1cnc(N2CCC3(CCO3)C2)nc1] | 1H NMR (399 MHz, DMSO) δ 8.32 (s, 2H), 7.60- 7.53 (m, 2H), 7.47 (dd, J = 8.9, 7.1 Hz, 1H), 5.88 (d, J = 5.9 Hz, 1H), 5.26 (s, 2H), 3.79-3.65 (m, 2H), 3.65- 3.46 (m, 3H), 2.93 (ddd, J = 9.3, 6.1, 3.3 Hz, 1H), 2.15- 1.94 (m, 2H), 1.82-1.70 (m, 2H). | 366 |
| 163 | 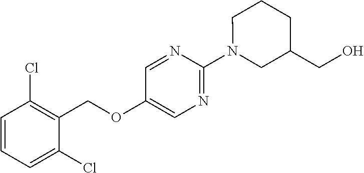 [SMILES: OCC1CCCN(c2ncc(OCc3c(Cl)cccc3Cl)cn2)C1] | 1H NMR (399 MHz, DMSO) δ 8.26 (s, 2H), 7.60- 7.52 (m, 2H), 7.47 (dd, J = 8.9, 7.1 H, 1H), 5.23 (s, 2H), 4.62-4.49 (m, 2H), 4.49-4.38 (m, 1H), 3.31- 3.20 (m, 1H), 2.82 (ddd, J = 12.9, 11.7, 2.9 Hz, 1H), 2.58 (dd, J = 13.0, 10.6 Hz, 1H), 1.79-1.63 (m, 2H), 1.63-1.47 (m, 1H), 1.45-1.31 (m, 1H), 1.25-1.11 (m, 1H). | 368 |
| 164 | 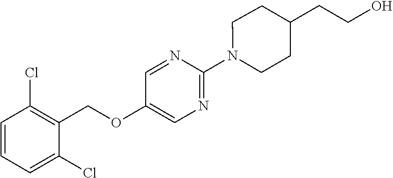 [SMILES: OCCC1CCN(c2ncc(OCc3c(Cl)cccc3Cl)cn2)CC1] | 1H NMR (399 MHz, DMSO) δ 8.26 (s, 2H), 7.59- 7.53 (m, 2H), 7.74 (dd, J = 9.0, 7.1 Hz, 1H), 5.23 (s, 2H), 4.57-4.47 (m, 2H), 4.37 (t, J = 5.1 Hz, 1H), 3.46 (td, J = 6.5, 4.6 Hz, 2H), 2.86- 2.74 (m, 2H), 1.75-1.57 (m, 3H), 1.37 (q, J = 6.6 Hz, 2H), 1.13-0.99 (m, 2H). | 382 |
| 165 | 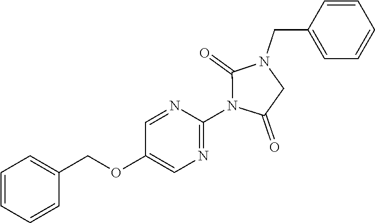 [SMILES: O=C1CN(Cc2ccccc2)C(=O)N1c1ncc(OCc2ccccc2)cn1] | 1H NMR (399 MHz, DMSO) δ 8.77 (s, 2H), 7.54- 7.47 (m, 2H), 7.47-7.28 (m, 8H), 5.35 (s, 2H), 4.56 (s, 2H), 4.16 (s, 2H). | 375 |
| 166 | 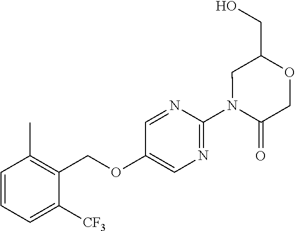 [SMILES: Cc1cccc(C(F)(F)F)c1COc1cnc(N2CC(CO)OCC2=O)nc1] | 1H NMR (400 MHz, DMSO-d6) δ 8.51 (s, 2H), 8.03 (d, J = 3.7 Hz, 1H), 7.64 (t, J = 7.0 Hz, 2H), 7.55 (t, J = 7.7 Hz, 1H), 5.22 (s, 2H), 4.34 (d, J = 5.1 Hz, 2H), 4.18- 4.09 (m, 1H), 4.07 (s, 2H), 3.29-3.15 (m, 2H), 2.44 (s, 3H). | 398 |
| 167 | 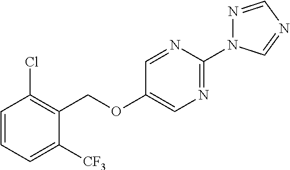 [SMILES: FC(F)(F)c1cccc(Cl)c1COc1cnc(-n2cncn2)nc1] | 1H NMR (400 MHz, DMSO) δ 9.35 (s, 1H), 8.87 (s, 2H), 8.29 (s, 1H), 7.97 (d, J = 7.7 Hz, 1H), 7.89 (d, J = 7.8 Hz, 1H), 7.74 (t, J = 7.8 Hz, 1H), 5.48 (s, 2H). | 356 |
| 168 | 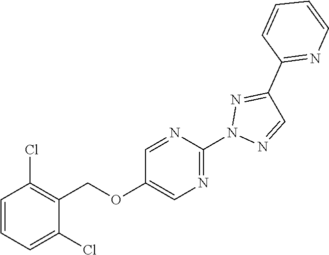 [SMILES: Clc1cccc(Cl)c1COc1cnc(-n2ncc(-c3ccccn3)n2)nc1] | 1H NMR (399 MHz, DMSO) δ 8.92 (d, J = 1.6 Hz, 2H), 8.72 (dt, J = 4.9, 1.3 Hz, 1H), 8.66 (d, J = 1.4 Hz, 1H), 8.11 (dd, J = 7.8, 1.2 Hz, 1H), 7.99 (td, J = 7.7, 1.8 Hz, 1H), 7.63 (d, J = 7.8 Hz, 2H), 7.58-7.45 (m, 2H), 5.55 (s, 2H) | 399 |
| 169 | 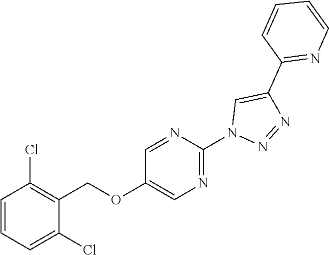 [SMILES: Clc1cccc(Cl)c1COc1cnc(-n2cc(-c3ccccn3)nn2)nc1] | 1H NMR (399 MHz, DMSO) δ 9.12 (s, 1H), 8.96 (s, 2H), 8.69 (ddd, J = 4.8, 1.8, 0.9 Hz, 1H), 8.17 (dt, J = 8.0, 1.2 Hz, 1H), 7.97 (td, J = 7.7, 1.8 Hz, 1H), 7.67- 7.60 (m, 2H), 7.54 (dd, J = 9.0, 7.1 Hz, 1H), 7.43 (ddd, J = 7.6, 4.8, 1.3 Hz, 1H), 5.56 (s, 2H), 3.31 (s, 1H) | 399 |
| 170 | 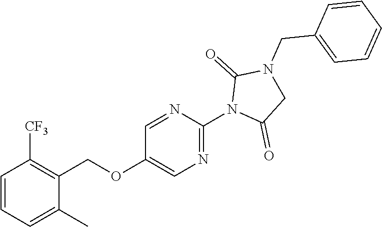 [SMILES: Cc1cccc(C(F)(F)F)c1COc1cnc(N2C(=O)CN(Cc3ccccc3)C2=O)nc1] | 1H NMR (399 MHz, DMSO) δ 8.87 (s, 2H), 7.67 (t, J = 7.7 Hz, 2H), 7.57 (t, J = 7.7 Hz, 1H), 7.43-7.30 (m, 5H), 5.40 (s, 2H), 4.57 (s, 2H), 4.18 (s, 2H), 2.46 (s, 3H) | 457 |
| 171 | 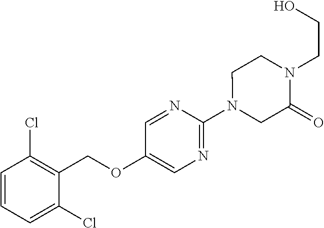 [SMILES: O=C1CN(c2ncc(OCc3c(Cl)cccc3Cl)cn2)CCN1CCO] | 1H NMR (399 MHz, DMSO) δ 8.36 (s, 2H), 7.57 (dd, J = 8.0, 0.9 Hz, 2H), 7.48 (dd, J = 8.9, 7.1 Hz, 1H), 5.27 (s, 2H), 4.74 (t, J = 5.5 Hz, 1H), 4.17 (s, 2H), 3.93-3.86 (m, 2H), 3.58- 3.47 (m, 4H), 3.40 (t, J = 5.8 Hz, 2H). | 397 |
| 172 | 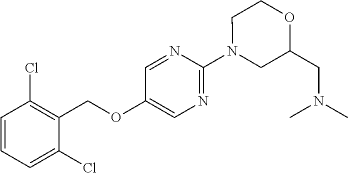 [SMILES: CN(C)CC1CN(c2ncc(OCc3c(Cl)cccc3Cl)cn2)CCO1] | 1H NMR (399 MHz, DMSO) δ 8.32 (s, 2H), 7.60- 7.53 (m, 2H), 7.47 (dd, J = 8.9, 7.1 Hz, 1H), 5.26 (s, 2H), 4.44 (ddd, J = 13.2, 2.7, 1.6 Hz, 1H), 4.26 (ddt, J = 13.1, 2.9, 1.5 Hz, 1H), 3.90 (ddd, J = 11.4, 3.5, 1.4 Hz, 1H), 3.59-3.45 (m, 2H), 2.90 (ddd, J = 13.1, 11.7, 3.5 Hz, 1H), 2.59 (dd, J = 13.2, | 397 |
| | | 10.4 Hz, 1H), 2.42-2.28 (m, | |
| | | 2H), 2.19 (s, 6H) | |
| 173 | 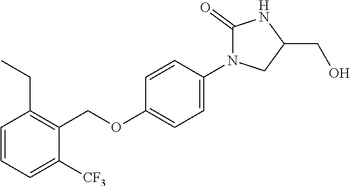 [SMILES: CCc1cccc(C(F)(F)F)c1COc1ccc(N2CC(CO)NC2=O)cc1] | 1H NMR (400 MHz, DMSO) δ 7.65 (d, J = 7.8 Hz, 2H), 7.61-7.53 (m, 1H), 7.48 (d, J = 9.2 Hz, 2H), 7.00 (d, J = 9.2 Hz, 2H), 6.93 (s, 1H), 5.07 (s, 2H), 4.97 (s, 1H), 3.88 (t, J = 8.9 Hz, 1H), 3.77-3.63 (m, 1H), 3.58 (dd, J = 9.0, 5.4 Hz, 1H), 3.45-3.38 (m, 2H), 2.75 (q, J = 7.6 Hz, 2H), 1.21 (t, J = 7.6 Hz, 3H). | 395 |
| 174 | 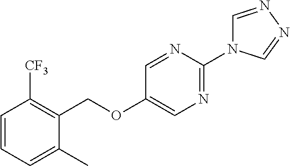 [SMILES: Cc1cccc(C(F)(F)F)c1COc1cnc(-n2cnnc2)nc1] | 1H NMR (400 MHz, DMSO) δ 9.24 (s, 2H), 8.85 (s, 2H), 7.67 (t, J = 7.3 Hz, 2H), 7.57 (t, J = 7.8 Hz, 1H), 5.38 (s, 2H), 2.45 (s, 3H). | 336 |
| 175 | 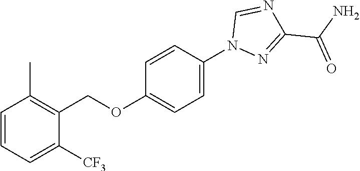 [SMILES: Cc1cccc(C(F)(F)F)c1COc1ccc(-n2cnc(C(N)=O)n2)cc1] | 1H NMR (399 MHz, DMSO) δ 9.28 (s, 1H), 7.95 (s, 1H), 7.89-7.81 (m, 2H), 7.71-7.62 (m, 3H), 7.55 (t, J = 7.8 Hz, 1H), 7.33-7.24 (m, 2H), 5.21 (d, J = 1.4 Hz, 2H), 2.45 (s, 3H) | 377 |
| 176 | 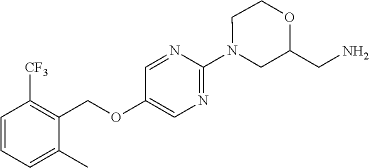 [SMILES: Cc1cccc(C(F)(F)F)c1COc1cnc(N2CCOC(CN)C2)nc1] | 1H NMR (399 MHz, DMSO) δ 8.34 (s, 2H), 7.67- 7.59 (m, 2H), 7.53 (t, J = 7.7 Hz, 1H), 5.14 (d, J = 1.5 Hz, 2H), 4.41 (ddd, J = 13.0, 2.7, 1.5 Hz, 1H), 4.28 (ddt, J = 13.1, 3.0, 1.5 Hz, 1H), 3.96 (ddd, J = 11.4, 3.6, 1.5 Hz, 1H), 3.59-3.43 (m, 2H), 2.92 (ddd, J = 13.2, 11.7, 3.5 Hz, 1H), 2.88-2.71 (m, | 383 |
| | | 2H), 2.65 (dd, J = 13.1, 10.5 | |
| | | Hz, 1H), 2.44 (s, 3H). | |
| 177 | 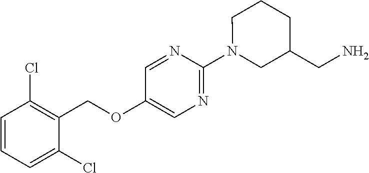 [SMILES: NCC1CCCN(c2ncc(OCc3c(Cl)cccc3Cl)cn2)C1] | 1H NMR (399 MHz, DMSO) δ 8.25 (s, 2H), 7.59-7.54 (m, 2H), 7.47 (dd, J = 9.0, 7.1 Hz, 1H), 5.23 (s, 3H), 4.50 (ddt, J = 12.9, 3.7, 1.6 Hz, 1H), 4.38 (dt, J = 13.0, 3.9 Hz, 1H), 2.89 (ddd, J = 12.9, 11.4, 3.1 Hz, 1H), 2.63 (dd, J = 12.9, 10.3 Hz, 1H), 2.46 (d, J = 6.6 Hz, 2H), 1.87-1.78 (m, 1H), 1.66 (dt, J = 13.2, 3.6 Hz, 1H), 1.39 (dddd, J = 20.4, | 367 |
| | | 15.6, 9.9, 7.7 Hz, 2H), 1.21-1.09 | |
| | | (m, 1H). | |
| 178 | 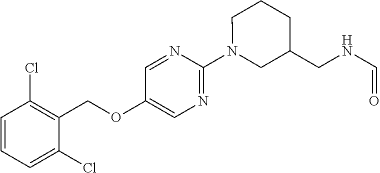 [SMILES: O=CNCC1CCCN(c2ncc(OCc3c(Cl)cccc3Cl)cn2)C1] | 1H NMR (399 MHz, DMSO) δ 8.19 (s, 2H), 8.02 (d, J = 6.6 Hz, 1H), 7.99 (d, J = 1.7 Hz, 1H), 7.52-7.46 (m, 2H), 7.39 (dd, J = 9.0, 7.1 Hz, 1H), 5.16 (s, 2H), 4.45-4.28 (m, 2H), 3.04- 2.88 (m, 2H), 2.84-2.73 (m, 1H), 2.54 (dd, J = 13.0, 10.5 Hz, 1H), 1.76-1.65 (m, 1H), 1.65-1.57 (m, 1H), | 395 |
| | | 1.57-1.44 (m, 1H), 1.39- | |
| | | 1.22 (m, 1H), 1.22-1.04 (m, 1H). | |
| 179 | 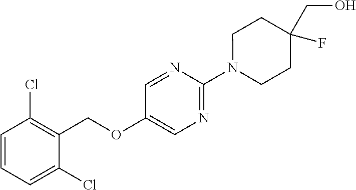 [SMILES: OCC1(F)CCN(c2ncc(OCc3c(Cl)cccc3Cl)cn2)CC1] | 1H NMR (399 MHz, DMSO) δ 8.30 (s, 2H), 7.59- 7.53 (m, 2H), 7.47 (dd, J = 8.9, 7.1 Hz, 1H), 5.25 (s, 2H), 4.97 (t, J = 6.0 Hz, 1H), 4.36-4.27 (m, 2H), 3.43 (dd, J = 20.0, 5.7 Hz, 2H), 3.21 (ddd, J = 13.2, 11.6, 3.2 Hz, 2H), 1.83-1.52 (m, 4H). | 386 |
| 180 | 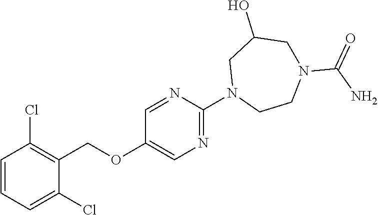 [SMILES: NC(=O)N1CCN(c2ncc(OCc3c(Cl)cccc3Cl)cn2)CC(O)C1] | 1H NMR (399 MHz, DMSO) δ 8.27 (s, 2H), 7.60- 7.52 (m, 2H), 7.46 (dd, J = 8.9, 7.1 Hz, 1H), 5.84 (s, 2H), 5.30-5.24 (m, 1H), 5.23 (s, 2H), 4.29 (ddt, J = 13.8, 4.7, 2.0 Hz, 2H), 3.86 (s, 1H), 3.70-3.35 (m, 2H), 3.29-3.06 (m, 2H), 3.00 (dd, J = 13.8, 9.0 Hz, 1H) | 414 |
| 181 | 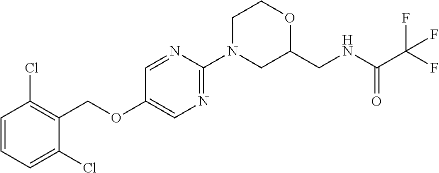 [SMILES: O=C(NCC1CN(c2ncc(OCc3c(Cl)cccc3Cl)cn2)CCO1)C(F)(F)F] | 1H NMR (399 MHz, DMSO) δ 9.62 (t, J = 5.8 Hz, 1H), 8.33 (s, 2H), 7.56 (dd, J = 8.1, 0.9 Hz, 2H), 7.47 (dd, J = 8.9, 7.1 Hz, 1H), 5.26 (s, 2H), 4.36 (ddd, J = 13.1, 2.7, 1.4 Hz, 1H), 4.24 (ddt, J = 13.1, 2.9, 1.5 Hz, 1H), 3.94 (ddd, J = 11.4, 3.5, 1.5 Hz, 1H), 3.65-3.54 (m, 1H), 3.50 (td, J = 11.5, 2.8 Hz, | 465 |
| | | 1H), 3.44-3.27 (m, 2H), | |
| | | 2.93 (ddd, J = 13.2, 11.6, 3.5 | |
| | | Hz, 1H), 2.66 (dd, J = 13.1, | |
| | | 10.4 Hz, 1H) | |
| 182 | 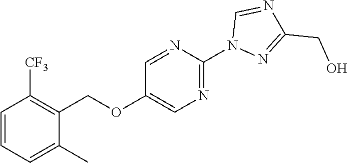 [SMILES: Cc1cccc(C(F)(F)F)c1COc1cnc(-n2cnc(CO)n2)nc1] | 1H NMR (399 MHz, DMSO) δ 9.20 (s, 1H), 8.76 (s, 2H), 7.65-7.56 (m, 2H), 7.51 (t, J = 7.7 Hz, 1H), 5.37 (t, J = 6.1 Hz, 1H), 5.34- 5.30 (m, 2H), 4.48 (d, J = 6.0 Hz, 2H), 2.39 (s, 3H) | 366 |
| 183 | 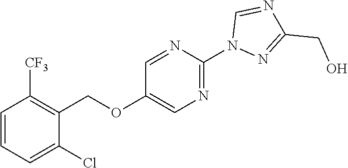 [SMILES: OCc1ncn(-c2ncc(OCc3c(Cl)cccc3C(F)(F)F)cn2)n1] | 1H NMR (399 MHz, DMSO) δ 9.20 (s, 1H), 8.79 (s, 2H), 7.94-7.87 (m, 1H), 7.82 (dd, J = 8.2, 1.2 Hz, 1H), 7.67 (td, J = 8.0, 1.0 Hz, 1H), 5.40 (d, J = 1.3 Hz, 2H), 5.37 (t, J = 6.1 Hz, 1H), 4.48 (d, J = 6.2 Hz, 2H) | 386 |
| 184 | 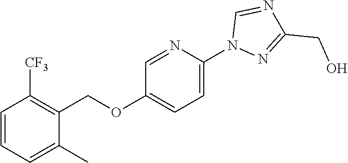 [SMILES: Cc1cccc(C(F)(F)F)c1COc1ccc(-n2cnc(CO)n2)nc1] | 1H NMR (399 MHz, DMSO) δ 9.18 (s, 1H), 8.35 (dd, J = 2.8, 0.9 Hz, 1H), 7.89-7.78 (m, 2H), 7.66 (t, J = 7.3 Hz, 2H), 7.60-7.52 (m, 1H), 5.41 (t, J = 6.0 Hz, 1H), 5.28 (br. s, 2H), 4.55 (d, J = 6.0 Hz, 2H), 2.45 (s, 3H) | 365 |
| 185 | 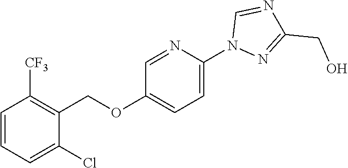 [SMILES: OCc1ncn(-c2ccc(OCc3c(Cl)cccc3C(F)(F)F)cn2)n1] | 1H NMR (399 MHz, DMSO) δ 9.12 (s, 1H), 8.32- 8.27 (m, 1H), 7.89 (dd, J = 8.1, 1.2 Hz, 1H), 7.84-7.76 (m, 2H), 7.75 (d, J = 8.8 Hz, 1H), 7.70-7.61 (m, 1H), 5.36 (t, J = 6.1 Hz, 1H), 5.29 (s, 2H), 4.48 (d, J = 6.0 Hz, 2H) | 385 |
| 186 | 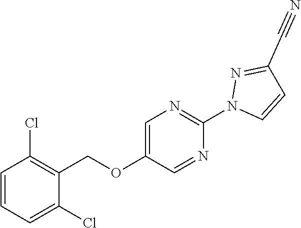 [SMILES: N#Cc1ccn(-c2ncc(OCc3c(Cl)cccc3Cl)cn2)n1] | 1H NMR (399 MHz, DMSO) δ 8.87 (s, 2H), 8.84 (d, J = 2.7 Hz, 1H), 7.64- 7.59 (m, 2H), 7.53 (dd, J = 9.0, 7.1 Hz, 1H), 7.28 (d, J = 2.8 Hz, 1H), 5.52 (s, 2H). | 346 |
| 187 | 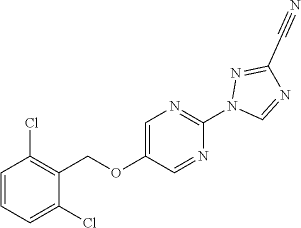 [SMILES: N#Cc1ncn(-c2ncc(OCc3c(Cl)cccc3Cl)cn2)n1] | 1H NMR (399 MHz, DMSO) δ 9.65 (s, 1H), 8.86 (s, 2H), 7.58-7.49 (m, 2H), 7.46 (dd, J = 9.0, 7.1 Hz, 1H), 5.47 (s, 2H). | 347 |
| 188 | 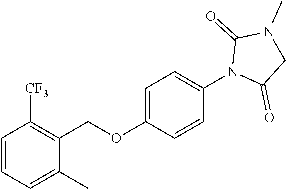 [SMILES: Cc1cccc(C(F)(F)F)c1COc1ccc(N2C(=O)CN(C)C2=O)cc1] | 1H NMR (399 MHz, DMSO) δ 7.64 (t, J = 8.3 Hz, 2H), 7.53 (t, J = 7.7 Hz, 1H), 7.32-7.24 (m, 2H), 7.19- 7.11 (m, 2H), 5.15 (s, 2H), 4.10 (s, 2H), 2.93 (s, 3H), 2.43 (s, 3H) | 379 |
| 189 | 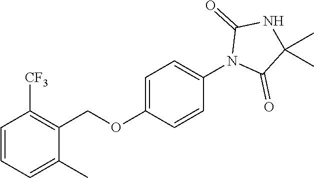 [SMILES: Cc1cccc(C(F)(F)F)c1COc1ccc(N2C(=O)NC(C)(C)C2=O)cc1] | 1H NMR (399 MHz, DMSO) δ 8.49 (s, 1H), 7.64 (t, J = 8.2 Hz, 2H), 7.57- 7.50 (m, 1H), 7.34-7.26 (m, 2H), 7.18-7.10 (m, 2H), 5.15 (s, 2H), 2.43 (s, 3H), 1.40 (s, 6H) | 393 |
| 190 | 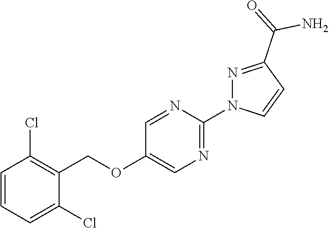 [SMILES: NC(=O)c1ccn(-c2ncc(OCc3c(Cl)cccc3Cl)cn2)n1] | 1H NMR (399 MHz, DMSO) δ 8.85 (s, 2H), 8.65 (d, J = 2.6 Hz, 1H), 7.77 (s, 1H), 7.66-7.62 (m, 2H), 7.55 (dd, J = 9.0, 7.1 Hz, 1H), 7.48 (s, 1H), 6.93 (d, J = 2.6 Hz, 1H), 5.53 (s, 2H). | 364 |
| 191 | 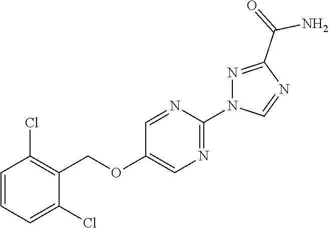 [SMILES: NC(=O)c1ncn(-c2ncc(OCc3c(Cl)cccc3Cl)cn2)n1] | 1H NMR (399 MHz, DMSO) δ 9.44 (s, 1H), 8.90 (s, 2H), 8.03 (s, 1H), 7.76 (s, 1H), 7.65-7.60 (m, 2H), 7.53 (dd, J = 9.0, 7.1 Hz, 1H), 5.54 (s, 2H). | 365 |
| 192 | 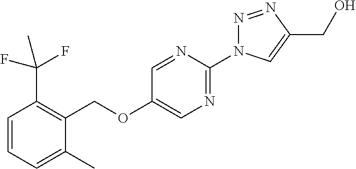 [SMILES: Cc1cccc(C(C)(F)F)c1COc1cnc(-n2cc(CO)nn2)nc1] | 1H NMR (400 MHz, DMSO) δ 8.59 (s, 1H), 7.83 (d, J = 8.8 Hz, 2H), 7.47- 7.42 (m, 3H), 7.26 (d, J = 9.2 Hz, 2H), 5.30 (t, J = 6.0 Hz, 1H), 5.21 (s, 2H), 4.60 (d, J = 5.6 Hz, 2H), 2.40 (s, 3H), 2.01 (t, J = 19.2 Hz, 3H). | 362 |
| 193 | 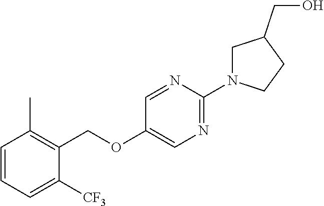 [SMILES: Cc1cccc(C(F)(F)F)c1COc1cnc(N2CCC(CO)C2)nc1] | 1H NMR (400 MHz, DMSO) δ 8.26 (s, 2H), 7.70- 7.57 (m, 2H), 7.53 (t, J = 7.8 Hz, 1H), 5.11 (s, 2H), 4.71 (t, J = 5.2 Hz, 1H), 3.63- 3.49 (m, 2H), 3.48-3.39 (m, 3H), 3.22 (dd, J = 11.0, 6.6 Hz, 1H), 2.46 (s, 3H), 2.39 (dt, J = 14.0, 7.0 Hz, 1H), 2.00 (td, J = 12.2, 7.4 Hz, 1H), 1.79-1.63 (m, 1H). | 368 |
| 194 | 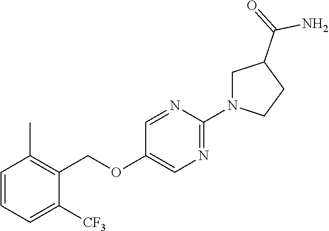 [SMILES: Cc1cccc(C(F)(F)F)c1COc1cnc(N2CCC(C(N)=O)C2)nc1] | 1H NMR (400 MHz, DMSO) δ 8.27 (s, 2H), 7.64-7.61 (m, 2H), 7.52 (t, J = 7.6 Hz, 1H), 7.46 (s, 1H), 6.93 (s, 1H), 5.11 (s, 2H), 3.69-3.64 (m, 1H), 3.58-3.55 (m, 1H), 3.51-3.46 (m, 1H), 3.44-3.38 (m, 1H), 3.01 (t, J = 7.6 Hz, 1H), 2.45 (s, 3H), 2.14-2.06 (m, 2H). | 381 |
| 195 | 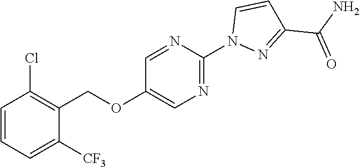 [SMILES: NC(=O)c1ccn(-c2ncc(OCc3c(Cl)cccc3C(F)(F)F)cn2)n1] | 1H NMR (399 MHz, DMSO) δ 8.84 (s, 2H), 8.63 (d, J = 2.6 Hz, 1H), 7.98 (dd, J = 8.2, 1.2 Hz, 1H), 7.89 (dd, J = 8.0, 1.2 Hz, 1H), 7.75 (td, J = 8.0, 6.8, 4.0 Hz, 2H), 7.46 (s, 1H), 6.91 (d, J = 2.6 Hz, 1H), 5.47 (s, 2H) | 398 |
| 196 | 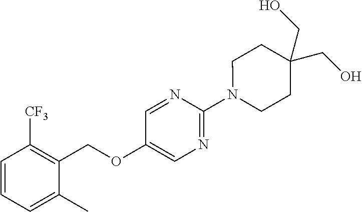 [SMILES: Cc1cccc(C(F)(F)F)c1COc1cnc(N2CCC(CO)(CO)CC2)nc1] | 1H NMR (399 MHz, DMSO) δ 8.27 (s, 2H), 7.67- 7.60 (m, 2H), 7.53 (t, J = 7.7 Hz, 1H), 5.12 (d, J = 1.3 Hz, 2H), 4.40 (t, J = 5.4 Hz, 2H), 3.70-3.61 (m, 4H), 3.35 (d, J = 5.4 Hz, 4H), 2.46 (s, 3H), 1.42-1.34 (m, 4H). | 412 |
| 197 | 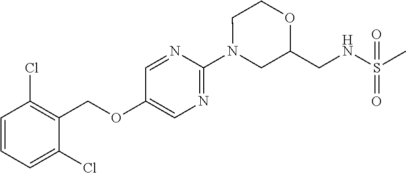 [SMILES: CS(=O)(=O)NCC1CN(c2ncc(OCc3c(Cl)cccc3Cl)cn2)CCO1] | 1H NMR (399 MHz, DMSO) δ 8.33 (s, 2H), 7.61- 7.53 (m, 2H), 7.47 (dd, J = 8.9, 7.1 Hz, 1H), 7.21 (t, J = 6.3 Hz, 1H), 5.26 (s, 2H), 4.44 (ddd, J = 13.2, 2.7, 1.5 Hz, 1H), 4.26 (ddt, J = 13.2, 3.0, 1.5 Hz, 1H), 3.94 (ddd, J = 11.5, 3.5, 1.4 Hz, 1H), 3.56- 3.43 (m, 2H), 3.16-3.00 (m, 2H), 2.92 (s, 3H), 2.98- | 449 |
| | | 2.86 (m, 1H), 2.64 (dd, J = | |
| | | 13.1, 10.5 Hz, 1H) | |
| 198 | 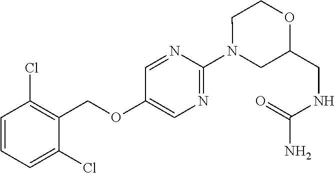 [SMILES: NC(=O)NCC1CN(c2ncc(OCc3c(Cl)cccc3Cl)cn2)CCO1] | 1H NMR (399 MHz, DMSO) δ 8.32 (s, 2H), 7.56 (dd, J = 8.0, 0.9 Hz, 2H), 7.47 (dd, J = 8.9, 7.1 Hz, 1H), 6.10 (t, J = 5.9 Hz, 1H), 5.50 (s, 2H), 5.25 (s, 2H), 4.36 (ddd, J = 13.2, 2.7, 1.5 Hz, 1H), 4.26 (dp, J = 13.0, 1.4 Hz, 1H), 3.93 (ddd, J = 11.4, 3.6, 1.4 Hz, 1H), 3.49 (td, J = 11.6, 2.8 Hz, 1H), | 414 |
| | | 3.40 (dtd, J = 10.6, 5.8, 2.6 | |
| | | Hz, 1H), 3.11 (td, J = 6.0, 2.4 | |
| | | Hz, 2H), 2.89 (ddd, J = 13.1, | |
| | | 11.7, 3.5 Hz, 1H), 2.60 (dd, | |
| | | J = 13.1, 10.5 Hz, 1H) | |
| 199 | 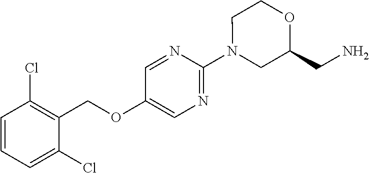 [SMILES: NC[C@H]1CN(c2ncc(OCc3c(Cl)cccc3Cl)cn2)CCO1] | 1H NMR (399 MHz, DMSO) δ 8.33 (s, 2H), 7.60- 7.53 (m, 2H), 7.47 (dd, J = 8.9, 7.1 Hz, 1H), 5.26 (s, 2H), 4.41 (ddd, J = 13.0, 2.7, 1.4 Hz, 1H), 4.27 (dp, J = 13.1, 1.5 Hz, 1H), 3.96 (ddd, J = 11.4, 3.5, 1.5 Hz, 1H), 3.57- 3.45 (m, 2H), 2.98-2.82 (m, 2H), 2.77 (d, J = 13.1, 7.5 Hz, 1H), 2.66 (dd, J = 13.0, 10.5 Hz, 1H). | 369 |
| 200 | 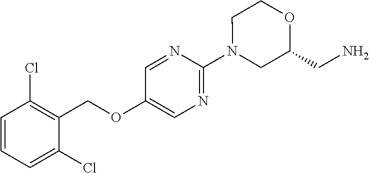 [SMILES: NC[C@@H]1CN(c2ncc(OCc3c(Cl)cccc3Cl)cn2)CCO1] | 1H NMR (399 MHz, DMSO) δ 8.33 (s, 2H), 7.60- 7.53 (m, 2H), 7.47 (dd, J = 8.9, 7.1 Hz, 1H), 5.26 (s, 2H), 4.41 (ddd, J = 13.0, 2.6, 1.4 Hz, 1H), 4.27 (ddt, J = 13.2, 2.8, 1.4 Hz, 1H), 3.96 (ddd, J = 11.4, 3.6, 1.5 Hz, 1H), 3.53 (dd, J = 11.6, 2.8 Hz, 1H), 3.48 (td, J = 7.3, 3.8 Hz, 1H), 2.98-2.80 (m, | 369 |
| | | 2H), 2.76 (dd, J = 13.1, 7.4 | |
| | | Hz, 1H), 2.65 (dd, J = 13.0, | |
| | | 10.5 Hz, 1H). | |
| 201 | 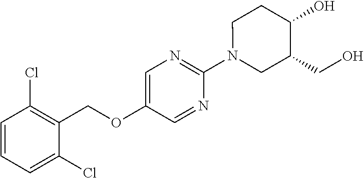 [SMILES: OC[C@@H]1CN(c2ncc(OCc3c(Cl)cccc3Cl)cn2)CC[C@@H]1O] | 1H NMR (399 MHz, DMSO) δ 8.26 (s, 2H), 7.56 (dd, J = 8.1, 0.9 Hz, 2H), 7.47 (dd, J = 9.0, 7.1 Hz, 1H), 5.23 (s, 2H), 4.56 (d, J = 3.8 Hz, 1H), 4.39 (t, J = 5.3 Hz, 1H), 4.28-4.11 (m, 2H), 3.92 (q, J = 3.4 Hz, 1H), 3.49 (ddd, J = 10.8, 5.9, 5.0 Hz, 1H), 3.03 (dd, J = 12.8, 10.8 Hz, 1H), 1.69-1.48 (m, 2H), 3.41-3.18 (m, 2H) | 384 |
| 202 | 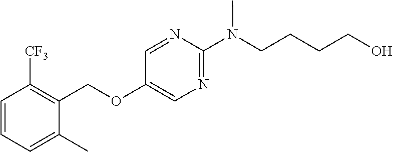 [SMILES: Cc1cccc(C(F)(F)F)c1COc1cnc(N(C)CCCCO)nc1] | 1H NMR (399 MHz, DMSO) δ 8.27 (s, 2H), 7.67- 7.59 (m, 2H), 7.57-7.49 (m, 1H), 5.10 (d, J = 1.4 Hz, 2H), 4.38 (t, J = 5.2 Hz, 1H), 3.56 (t, J = 7.4 Hz, 2H), 3.41 (td, J = 6.5, 5.3 Hz, 2H), 3.05 (s, 3H), 2.46 (s, 3H), 1.62- 1.50 (m, 2H), 1.46-1.34 (m, 2H). | 370 |
| 203 | 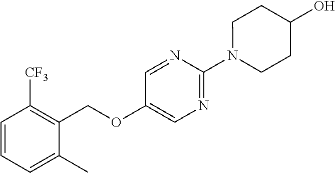 [SMILES: Cc1cccc(C(F)(F)F)c1COc1cnc(N2CCC(O)CC2)nc1] | 1H NMR (399 MHz, DMSO) δ 8.28 (s, 2H), 7.67- 7.59 (m, 2H), 7.57-7.49 (m, 1H), 5.12 (d, J = 1.4 Hz, 2H), 4.69 (d, J = 4.3 Hz, 1H), 4.25-4.15 (m, 2H), 3.70 (tt, J = 8.5, 4.2 Hz, 1H), 3.18 (ddd, J = 13.3, 10.0, 3.1 Hz, 2H), 2.45 (s, 3H), 1.81-1.71 (m, 2H), 1.32 (tdd, J = 13.1, 9.4, 3.9 Hz, 2H). | 368 |
| 204 | 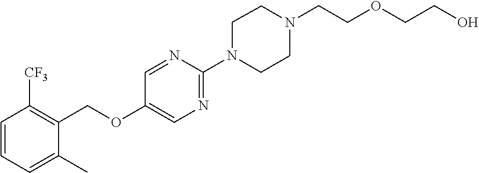 [SMILES: Cc1cccc(C(F)(F)F)c1COc1cnc(N2CCN(CCOCCO)CC2)nc1] | 1H NMR (399 MHz, DMSO) δ 8.31 (s, 2H), 7.67- 7.59 (m, 2H), 7.53 (t, J = 7.8 Hz, 1H), 5.13 (d, J = 1.3 Hz, 2H), 3.63 (dd, J = 6.1, 4.0 Hz, 4H), 3.56 (t, J = 5.9 Hz, 2H), 3.53-3.46 (m, 2H), 3.46-3.39 (m, 2H), 3.25 (s, 0H), 2.57-2.46 (m, 6H), 2.45 (s, 3H). | 441 |
| 205 | 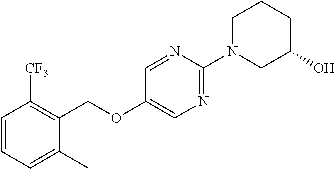 [SMILES: Cc1cccc(C(F)(F)F)c1COc1cnc(N2CCC[C@H](O)C2)nc1] | 1H NMR (399 MHz, DMSO) δ 8.27 (s, 2H), 7.68- 7.59 (m, 2H), 7.57-7.49 (m, 1H), 5.12 (d, J = 1.3 Hz, 2H), 4.85 (d, J = 4.4 Hz, 1H), 4.39 (ddt, J = 12.5, 4.3, 1.6 Hz, 1H), 4.24 (ddd, J = 13.0, 4.6, 2.8 Hz, 1H), 3.44 (dh, J = 8.8, 4.3 Hz, 1H), 2.94 (ddd, J = 13.1, 10.6, 3.0 Hz, 1H), 2.79 (dd, J = 12.5, 9.2 | 368 |
| | | Hz, 1H), 2.45 (s, 3H), 1.90 | |
| | | (dd, J = 7.1, 3.1 Hz, 1H), | |
| | | 1.76-1.64 (m, 1H), 1.37 | |
| | | (ddd, J = 10.9, 7.2, 2.8 Hz, 2H). | |
| 206 | 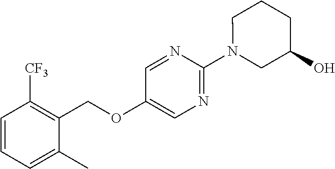 [SMILES: Cc1cccc(C(F)(F)F)c1COc1cnc(N2CCC[C@@H](O)C2)nc1] | 1H NMR (399 MHz, DMSO) δ 8.27 (s, 2H), 7.68- 7.59 (m, 2H), 7.57-7.49 (m, 1H), 5.12 (d, J = 1.3 Hz, 2H), 4.85 (d, J = 4.4 Hz, 1H), 4.39 (ddt, J = 12.5, 4.4, 1.6 Hz, 1H), 4.29-4.19 (m, 1H), 3.44 (dp, J = 8.9, 4.4 Hz, 1H), 3.00-2.88 (m, 1H), 2.79 (dd, J = 12.5, 9.2 Hz, 1H), 2.45 (s, 3H), 1.93- | 368 |
| | | 1.87 (m, 1H), 1.70 (dqd, J = | |
| | | 11.6, 7.0, 4.0 Hz, 1H), 1.46- | |
| | | 1.29 (m, 2H). | |
| 207 | 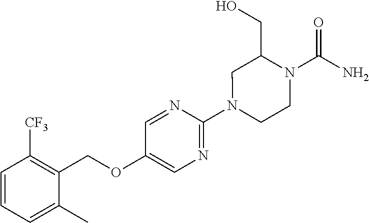 [SMILES: Cc1cccc(C(F)(F)F)c1COc1cnc(N2CCN(C(N)=O)C(CO)C2)nc1] | 1H NMR (399 MHz, DMSO) δ 8.32 (s, 2H), 7.64 (dd, J = 7.5, 5.9 Hz, 2H), 7.53 (t, J = 7.7 Hz, 1H), 5.95 (s, 2H), 5.16-5.11 (m, 2H), 4.71 (dd, J = 5.9, 4.7 Hz, 1H), 4.51 (dt, J = 13.0, 2.0 Hz, 1H), 4.29-4.20 (m, 1H), 4.04 (s, 1H), 3.84 (dd, J = 12.6, 3.9 Hz, 1H), 3.49- 3.33 (m, 2H), 3.09-2.88 (m, 3H), 2.45 (s, 3H) | 426 |
| 208 | 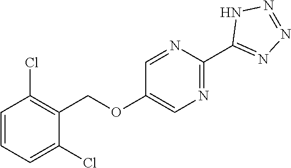 [SMILES: Clc1cccc(Cl)c1COc1cnc(-c2nnn[nH]2)nc1] | 1H NMR (400 MHz, DMSO) δ 8.55 (s, 1H), 7.66- 7.54 (m, 1H), 7.50 (dd, J = 9.2, 6.8 Hz, 1H), 5.45 (s, 1H). | 323 |
| 209 | 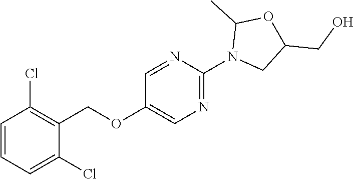 [SMILES: CC1OC(CO)CN1c1ncc(OCc2c(Cl)cccc2Cl)cn1] | 1H NMR (400 MHz, DMSO) δ 8.50 (s, 2H), 7.46 (d, J = 8.1 Hz, 2H), 7.30 (t, J = 7.9 Hz, 1H), 5.23 (t, J = 5.6 Hz, 1H), 4.66 (s, 1H), 4.15 (t, J = 9.6 Hz, 1H), 3.95 (dd, J = 9.8, 6.5 Hz, 1H), 3.66 (s, 1H), 3.56 (d, J = 12.3 Hz, 1H), 3.20-3.11 (m, 2H), 2.86 (t, J = 7.7 Hz, 2H). | 370 |
| 210 | 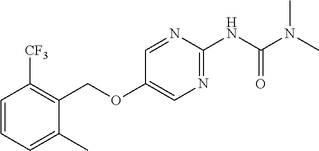 [SMILES: Cc1cccc(C(F)(F)F)c1COc1cnc(NC(=O)N(C)C)nc1] | 1H NMR (400.14 MHz, DMSO) δ 9.16 (s, 1H), 8.47 (s, 2H), 7.67-7.62 (m, 2H), 7.57-7.54 (m, 1H), 5.23 (s, 2H), 6.93 (s, 1H), 2.91 (s, 6H), 2.45 (s, 3H). | 355 |
| 211 | 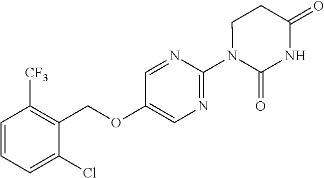 [SMILES: O=C1CCN(c2ncc(OCc3c(Cl)cccc3C(F)(F)F)cn2)C(=O)N1] | 1H NMR (400 MHz, DMSO) δ 10.52 (s, 1H), 8.70 (s, 2H), 7.96 (d, J = 7.9 Hz, 1H), 7.87 (d, J = 7.8 Hz, 1H), 7.73 (t, J = 8.2 Hz, 1H), 5.39 (s, 2H), 3.92 (t, J = 6.6 Hz, 2H), 2.71 (t, J = 6.5 Hz, 2H). | 401 |
| 212 | 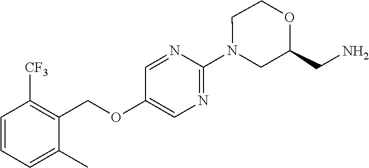 [SMILES: Cc1cccc(C(F)(F)F)c1COc1cnc(N2CCO[C@@H](CN)C2)nc1] | 1H NMR (399 MHz, DMSO) δ 8.34 (s, 2H), 7.67- 7.59 (m, 2H), 7.53 (t, J = 7.7 Hz, 1H), 5.14 (d, J = 1.2 Hz, 2H), 4.41 (ddd, J = 13.1, 2.7, 1.5 Hz, 1H), 4.28 (ddt, J = 13.2, 3.0, 1.5 Hz, 1H), 3.96 (ddd, J = 11.4, 3.5, 1.5 Hz, 1H), 3.50 (dtt, J = 12.4, 4.6, 2.7 Hz, 2H), 2.92 (ddd, J = 13.1, 11.7, 3.5 Hz, 1H), 2.87- | 383 |
| | | 2.71 (m, 2H), 2.65 (dd, J = | |
| | | 13.0, 10.5 Hz, 1H), 2.44 (s, 3H). | |
| 213 | 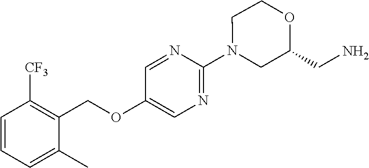 [SMILES: Cc1cccc(C(F)(F)F)c1COc1cnc(N2CCO[C@H](CN)C2)nc1] | 1H NMR (399 MHz, DMSO) δ 8.34 (s, 2H), 7.67- 7.59 (m, 2H), 7.57-7.49 (m, 1H), 5.14 (d, J = 1.4 Hz, 2H), 4.41 (ddd, J = 13.1, 2.6, 1.4 Hz, 1H), 4.28 (ddt, J = 13.2, 3.0, 1.5 Hz, 1H), 3.96 (ddd, J = 11.5, 3.5, 1.5 Hz, 1H), 3.57-3.44 (m, 2H), 2.93 (ddd, J = 13.1, 11.7, 3.5 Hz, 1H), 2.88-2.71 (m, | 383 |
| | | 2H), 2.66 (dd, J = 13.1, 10.5 | |
| | | Hz, 1H), 2.44 (s, 3H). | |
| 214 | 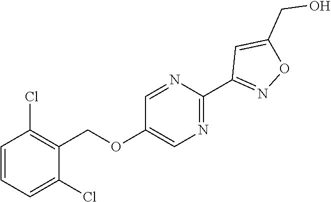 [SMILES: OCc1cc(-c2ncc(OCc3c(Cl)cccc3Cl)cn2)no1] | 1H NMR (399 MHz, DMSO) δ 8.86 (s, 2H), 7.63- 7.59 (m, 2H), 7.52 (dd, J = 9.0, 7.1 Hz, 1H), 6.86 (s, 1H), 5.75 (t, J = 6.0 Hz, 1H), 5.52 (s, 2H), 4.64 (d, J = 5.3 Hz, 2H) | 352 |
| 215 | 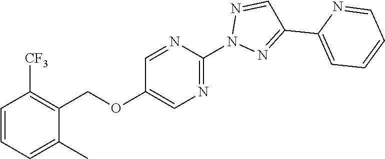 [SMILES: Cc1cccc(C(F)(F)F)c1COc1cnc(-n2ncc(-c3ccccn3)n2)nc1] | 1H NMR (399 MHz, DMSO) δ 8.91 (s, 2H), 8.73 (ddd, J = 4.9, 1.8, 1.0 Hz, 1H), 8.66 (s, 1H), 8.11 (dt, J = 7.9, 1.1 Hz, 1H), 7.99 (td, J = 7.7, 1.8 Hz, 1H), 7.73- 7.62 (m, 2H), 7.59 (t, J = 7.8 Hz, 1H), 7.50 (ddd, J = 7.5, 4.8, 1.2 Hz, 1H), 5.44 (s, 2H), 2.48 (s, 3H) | 413 |
| 216 | 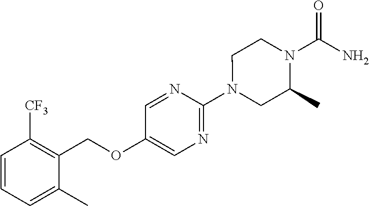 [SMILES: Cc1cccc(C(F)(F)F)c1COc1cnc(N2CCN(C(N)=O)[C@@H](C)C2)nc1] | 1H NMR (399 MHz, DMSO) δ 8.31 (s, 2H), 7.68- 7.60 (m, 2H), 7.53 (t, J = 7.8 Hz, 1H), 5.99 (s, 2H), 5.13 (d, J = 1.3 Hz, 2H), 4.43- 4.35 (m, 1H), 4.31 (dt, J = 12.9, 2.0 Hz, 1H), 4.23 (ddd, J = 10.5, 5.2, 3.1 Hz, 1H), 3.79 (dt, J = 12.9, 3.1 Hz, 1H), 3.11-2.95 (m, 2H), 2.86 (td, J = 12.3, 3.6 Hz, 1H), 2.45 (s, 3H), 1.03 (d, J = 6.6 Hz, 3H) | 410 |
| 217 | 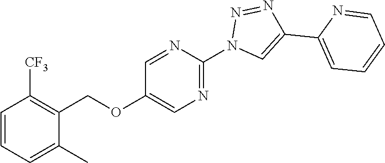 [SMILES: Cc1cccc(C(F)(F)F)c1COc1cnc(-n2cc(-c3ccccn3)nn2)nc1] | 1H NMR (399 MHz, DMSO) δ 9.12 (s, 1H), 8.95 (s, 2H), 8.69 (ddd, J = 4.8, 1.8, 1.0 Hz, 1H), 8.17 (dt, J = 8.0, 1.1 Hz, 1H), 7.97 (td, J = 7.7, 1.8 Hz, 1H), 7.69 (t, J = 7.4 Hz, 2H), 7.59 (t, J = 7.7 Hz, 1H), 7.43 (ddd, J = 7.6, 4.9, 1.2 Hz, 1H), 5.44 (s, 2H), 2.48 (s, 3H) | 413 |
| 218 | 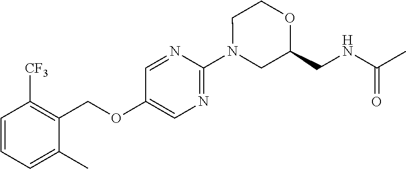 [SMILES: CC(=O)NC[C@H]1CN(c2ncc(OCc3c(C)cccc3C(F)(F)F)cn2)CCO1] | 1H NMR (399 MHz, DMSO) δ 8.33 (s, 2H), 8.03 (t, J = 5.9 Hz, 1H), 7.67- 7.59 (m, 2H), 7.52 (t, J = 7.9 Hz, 1H), 5.14 (d, J = 1.3 Hz, 2H), 4.35 (ddd, J = 13.0, 2.6, 1.4 Hz, 1H), 4.25 (dp, J = 13.2, 1.5 Hz, 1H), 3.93 (ddd, J = 11.4, 3.6, 1.5 Hz, 1H), 3.54-3.38 (m, 2H), 3.26- 3.11 (m, 2H), 2.90 (ddd, J = | 425 |
| | | 13.2, 11.7, 3.5 Hz, 1H), 2.60 | |
| | | (dd, J = 13.1, 10.5, Hz, 1H), | |
| | | 2.44 (s, 3H), 1.83 (s, 3H) | |
| 219 | 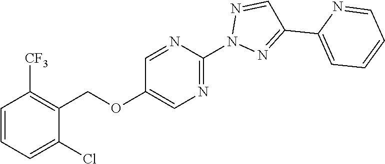 [SMILES: FC(F)(F)c1cccc(Cl)c1COc1cnc(-n2ncc(-c3ccccn3)n2)nc1] | 1H NMR (399 MHz, DMSO) δ 8.93 (s, 2H), 8.76- 8.62 (m, 1H), 8.66 (s, 1H), 8.11 (dt, J = 7.9, 1.1 Hz, 1H), 8.03-7.97 (m, 1H), 8.01- 7.90 (m, 1H), 7.90 (dd, J = 8.0, 1.2 Hz, 1H), 7.75 (td, J = 8.1, 0.9 Hz, 1H), 7.50 (ddd, J = 7.6, 4.8, 1.2 Hz, 1H), 5.52 (d, J = 1.2 Hz, 2H) | 433 |
| 220 | 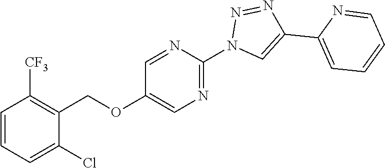 [SMILES: FC(F)(F)c1cccc(Cl)c1COc1cnc(-n2cc(-c3ccccn3)nn2)nc1] | 1H NMR (399 MHz, DMSO) δ 9.13 (s, 1H), 8.98 (s, 2H), 8.69 (ddd, J = 4.8, 1.8, 0.9 Hz, 1H), 8.17 (dt, J = 7.9, 1.1 Hz, 1H), 8.02- 7.93 (m, 2H), 7.90 (dd, J = 8.1, 1.2 Hz, 1H), 7.76 (td, J = 8.0, 0.9 Hz, 1H), 7.43 (ddd, J = 7.5, 4.8, 1.2 Hz, 1H), 5.53 (s, 2H) | 433 |
| 221 | 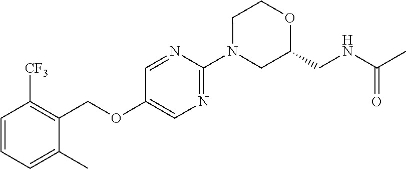 [SMILES: CC(=O)NC[C@@H]1CN(c2ncc(OCc3c(C)cccc3C(F)(F)F)cn2)CCO1] | 1H NMR (399 MHz, DMSO) δ 8.33 (s, 2H), 8.03 (t, J = 5.9 Hz, 1H), 7.67- 7.59 (m, 2H), 7.53 (t, J = 7.7 Hz, 1H), 5.14 (d, J = 1.3 Hz, 2H), 4.35 (ddd, J = 13.1, 2.6, 1.4 Hz, 1H), 4.25 (dp, J = 13.1, 1.5 Hz, 1H), 3.93 (ddd, J = 11.4, 3.5, 1.5 Hz, 1H), 3.54-3.39 (m, 2H), 3.18 (td, J = 5.9, 3.9 Hz, 2H), 2.90 | 425 |
| | | (ddd, J = 13.1, 11.7, 3.5 Hz, | |
| | | 1H), 2.60 (dd, J = 13.1, 10.5 | |
| | | Hz, 1H), 2.44 (s, 3H), 1.83 | |
| | | (s, 3H) | |
| 222 | 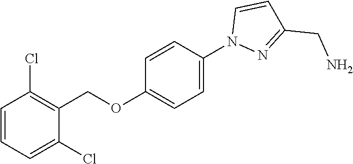 [SMILES: NCc1ccn(-c2ccc(OCc3c(Cl)cccc3Cl)cc2)n1] | 1H NMR (400 MHz, DMSO) δ 8.32 (d, J = 2.5 Hz, 1H), 7.78-7.69 (m, 2H), 7.58 (d, J = 8.0 Hz, 2H), 7.48 (dd, J = 8.9, 7.1 Hz, 1H), 7.26-7.11 (m, 2H), 6.48 (d, J = 2.4 Hz, 1H), 5.27 (s, 2H), 3.81 (s, 2H). | 348 |
| 224 | 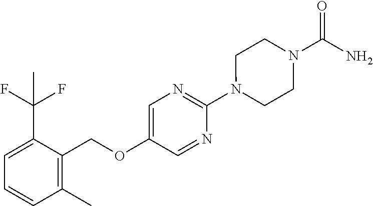 [SMILES: Cc1cccc(C(C)(F)F)c1COc1cnc(N2CCN(C(N)=O)CC2)nc1] | 1H NMR (400 MHz, MeOD) δ 8.23 (s, 2H), 7.47- 7.41 (m, 1H), 7.41-7.32 (m, 2H), 5.21 (s, 2H), 3.76- 5.73 (m, 4H), 3.50-3.46 (m, 4H), 2.46 (s, 3H), 1.97 (t, J = 18.5 Hz, 3H). | 392 |
| 225 | 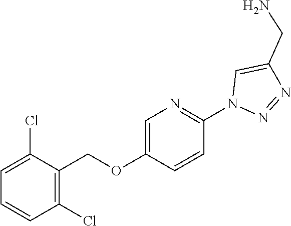 [SMILES: NCc1cn(-c2ccc(OCc3c(Cl)cccc3Cl)cn2)nn1] | 1H NMR (400 MHz, CDCl3) δ 8.40 (s, 1H), 8.24 (d, J = 2.6 Hz, 1H), 8.11 (d, J = 8.8 Hz, 1H), 7.54 (dd, J = 8.8, 2.8 Hz, 1H), 7.40 (d, J = 8.4 Hz, 2H), 7.29 (dd, J = 8.8, 7.4 Hz, 1H), 5.39 (s, 2H), 4.10 (s, 2H). | 350 |
| 226 | 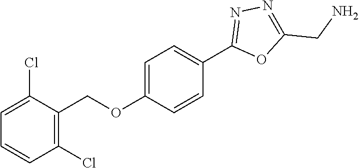 [SMILES: NCc1nnc(-c2ccc(OCc3c(Cl)cccc3Cl)cc2)o1] | 1H NMR (400 MHz, DMSO) δ 7.97 (d, J = 8.8 Hz, 2H), 7.59 (d, J = 8.0 Hz, 2H), 7.50 (dd, J = 8.8, 7.4 Hz, 1H), 7.29 (d, J = 8.8 Hz, 2H), 5.34 (s, 2H), 3.95 (s, 2H), 2.11 (s, 2H). | 350 |
| 227 | 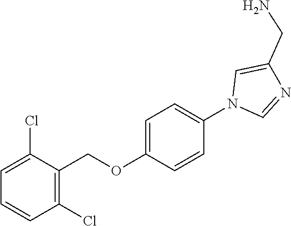 [SMILES: NCc1cn(-c2ccc(OCc3c(Cl)cccc3Cl)cc2)cn1] | 1H NMR (400 MHz, DMSO) δ 8.06 (s, 1H), 7.61- 7.51 (m, 4H), 7.49 (dd, J = 8.9, 7.1 Hz, 1H), 7.44 (s, 1H), 7.24-7.15 (m, 2H), 5.28 (s, 2H), 3.64 (s, 2H), 1.23 (s, 1H). | 348 |
| 228 | 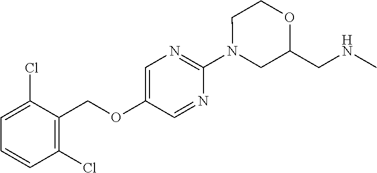 [SMILES: CNCC1CN(c2ncc(OCc3c(Cl)cccc3Cl)cn2)CCO1] | 1H NMR (399 MHz, DMSO) δ 8.32 (s, 2H), 7.61- 7.51 (m, 2H), 7.47 (dd, J = 8.9, 7.1 Hz, 1H), 5.26 (s, 2H), 4.41 (ddd, J = 13.1, 2.6, 1.5 Hz, 1H), 4.27 (dp, J = 13.1, 1.5 Hz, 1H), 3.92 (ddd, J = 11.4, 3.6, 1.4 Hz, 1H), 3.49 (ddt, J = 13.5, 6.1, 2.5 Hz, 2H), 2.90 (ddd, J = 13.1, 11.7, 3.5 Hz, 1H), 2.70-2.55 (m, 3H), | 383 |
| | | 2.30 (s, 3H). | |
| 229 | 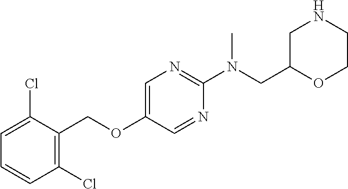 [SMILES: CN(CC1CNCCO1)c1ncc(OCc2c(Cl)cccc2Cl)cn1] | 1H NMR (399 MHz, DMSO) δ 8.27 (s, 2H), 7.61- 7.54 (m, 2H), 7.47 (dd, J = 8.4, 7.1 Hz, 1H), 5.23 (s, 2H), 3.75-3.66 (m, 1H), 3.67-3.54 (m, 2H), 3.54- 3.44 (m, 1H), 3.42-3.35 (m, 1H), 3.09 (s, 3H), 2.80-2.55 (m, 3H), 2.44-2.27 (m, 1H). | 383 |
| 230 | 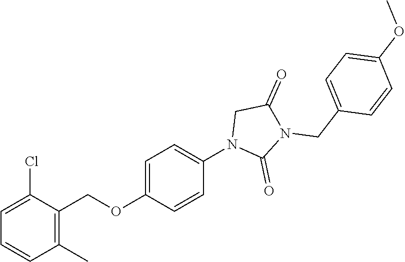 [SMILES: COc1ccc(CN2C(=O)CN(c3ccc(OCc4c(C)cccc4Cl)cc3)C2=O)cc1] | 1H NMR (399 MHz, DMSO) δ 7.63 (t, J = 8.2 Hz, 2H), 7.58-7.49 (m, 3H), 7.32-7.24 (m, 2H), 7.14- 7.05 (m, 2H), 6.94-6.86 (m, 2H), 5.11 (s, 2H), 4.57 (s, 2H), 4.54 (s, 2H), 3.73 (s, 3H), 2.41 (s, 3H) | 485 |
| 231 | 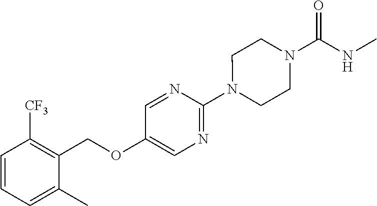 [SMILES: CNC(=O)N1CCN(c2ncc(OCc3c(C)cccc3C(F)(F)F)cn2)CC1] | 1H NMR (399 MHz, DMSO) δ 8.32 (s, 2H), 7.67- 7.59 (m, 2H), 7.57-7.48 (m, 1H), 6.50 (q, J = 5.2 Hz, 1H), 5.13 (d, J = 1.3 Hz, 2H), 3.65-3.57 (m, 4H), 3.39- 3.32 (m, 4H), 2.58 (d, J = 4.3 Hz, 3H), 2.44 (s, 3H) | 410 |
| 232 | 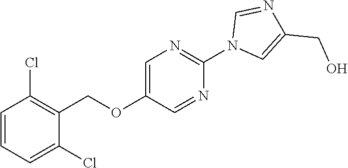 [SMILES: OCc1cn(-c2ncc(OCc3c(Cl)cccc3Cl)cn2)cn1] | 1H NMR (400 MHz, DMSO) δ 9.17 (s, 2H), 8.54 (d, J = 1.3 Hz, 1H), 7.79 (s, 1H), 7.58 (d, J = 8.1 Hz, 2H), 7.46 (d, J = 16.9 Hz, 1H), 7.38 (t, J = 7.7 Hz, 1H), 7.16 (d, J = 16.9 Hz, 1H), 5.09 (s, 1H), 4.44 (d, J = 5.2 Hz, 2H). | 351 |
| 233 | 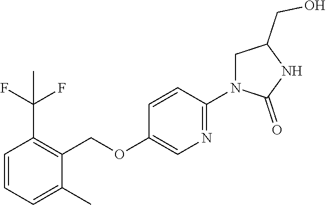 [SMILES: Cc1cccc(C(C)(F)F)c1COc1ccc(N2CC(CO)NC2=O)nc1] | 1H NMR (400 MHz, DMSO) δ 8.13-8.10 (m, 2H), 7.51 (dd, J = 9.2, 3.2 Hz, 1H), 7.46-7.39 (m, 3H), 7.18 (s, 1H), 5.16 (s, 2H), 4.93 (t, J = 5.6 Hz, 1H), 4.01-3.96 (m, 1H), 3.77 (dd, J = 10.8, 5.2 Hz, 1H), 3.71-3.65 (m, 1H), 3.43- 3.37 (m, 2H), 2.39 (s, 3H), 1.99 (t, J = 18.8 Hz, 3H). | 378 |
| 234 | 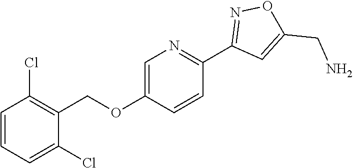 [SMILES: NCc1cc(-c2ccc(OCc3c(Cl)cccc3Cl)cn2)no1] | 1H NMR (400 MHz, DMSO) δ 8.52 (d, J = 2.8 Hz, 1H), 7.99 (d, J = 8.6 Hz, 1H), 7.72 (dd, J = 8.8, 2.8 Hz, 1H), 7.60 (d, J = 7.6 Hz, 2H), 7.51 (dd, J = 8.8, 7.2 Hz, 1H), 6.80 (s, 1H), 5.40 (s, 2H), 3.85 (s, 2H), 2.08 (s, 2H). | 350 |
| 235 | 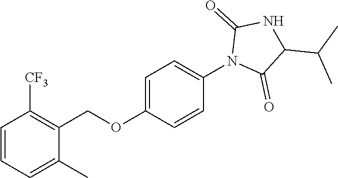 [SMILES: Cc1cccc(C(F)(F)F)c1COc1ccc(N2C(=O)NC(C(C)C)C2=O)cc1] | 1H NMR (399 MHz, DMSO) δ 8.48 (d, J = 1.4 Hz, 1H), 7.65 (t, J = 7.8 Hz, 2H), 7.55 (t, J = 7.8 Hz, 1H), 7.30- 7.21 (m, 2H), 7.19-7.11 (m, 2H), 5.18-5.13 (m, 2H), 4.13 (dd, J = 3.5, 1.2 Hz, 1H), 2.44 (s, 3H), 2.14 (pd, J = 6.8, 3.6 Hz, 1H), 1.03 (d, J = 7.0 Hz, 3H), 0.89 (d, J = 6.8 Hz, 3H) | 407 |
| 236 | 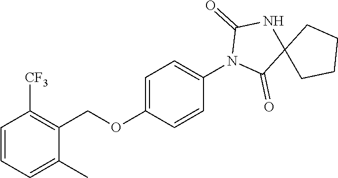 [SMILES: Cc1cccc(C(F)(F)F)c1COc1ccc(N2C(=O)NC3(CCCC3)C2=O)cc1] | 1H NMR (399 MHz, DMSO) δ 8.70 (s, 1H), 7.65 (t, J = 7.8 Hz, 2H), 7.59- 7.50 (m, 1H), 7.35-7.26 (m, 2H), 7.19-7.10 (m, 2H), 5.16 (d, J = 1.4 Hz, 2H), 2.44 (s, 3H), 2.07 (tdd, J = 9.8, 5.4, 3.9 Hz, 2H), 1.92- 1.71 (m, 6H) | 419 |
| 237 | 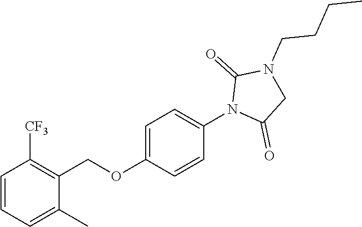 [SMILES: CCCCN1CC(=O)N(c2ccc(OCc3c(C)cccc3C(F)(F)F)cc2)C1=O] | 1H NMR (399 MHz, DMSO) δ 7.65 (t, J = 7.8 Hz, 2H), 7.55 (t, J = 7.7 Hz, 1H), 7.33-7.24 (m, 2H), 7.19- 7.11 (m, 2H), 5.18-5.13 (m, 2H), 4.12 (s, 2H), 3.35 (t, J = 7.2 Hz, 2H), 2.44 (s, 3H), 1.61-1.49 (m, 2H), 1.40- 1.26 (m, 2H), 0.92 (t, J = 7.3 Hz, 3H) | 421 |
| 238 | 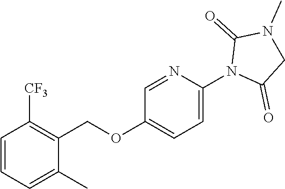 [SMILES: Cc1cccc(C(F)(F)F)c1COc1ccc(N2C(=O)CN(C)C2=O)nc1] | 1H NMR (399 MHz, DMSO) δ 8.23-8.18 (m, 1H), 8.12 (dd, J = 9.1, 0.6 Hz, 1H), 7.72-7.61 (m, 3H), 7.55 (t, J = 7.7 Hz, 1H), 5.19 (d, J = 1.3 Hz, 2H), 4.47 (s, 2H), 2.95 (s, 3H), 2.44 (s, 3H) | 380 |
| 239 | 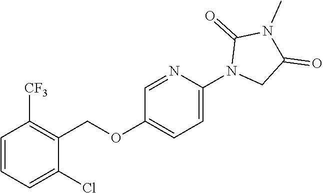 [SMILES: CN1C(=O)CN(c2ccc(OCc3c(Cl)cccc3C(F)(F)F)cn2)C1=O] | 1H NMR (399 MHz, DMSO) δ 8.21 (dd, J = 3.1, 0.7 Hz, 1H), 8.12 (dd, J = 9.0, 0.6 Hz, 1H), 7.94 (dd, J = 8.2, 1.2 Hz, 1H), 7.85 (dd, J = 8.0, 1.2 Hz, 1H), 7.75- 7.63 (m, 2H), 5.27 (d, J = 1.4 Hz, 2H), 4.47 (s, 2H), 2.94 (s, 3H) | 400 |
| 240 | 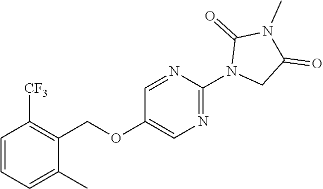 [SMILES: Cc1cccc(C(F)(F)F)c1COc1cnc(N2CC(=O)N(C)C2=O)nc1] | 1H NMR (399 MHz, DMSO) δ 8.65 (s, 2H), 7.69- 7.61 (m, 2H), 7.55 (t, J = 7.7 Hz, 1H), 5.30-5.26 (m, 2H), 4.49 (s, 2H), 2.93 (s, 3H), 2.44 (s, 3H). | 381 |
| 241 | 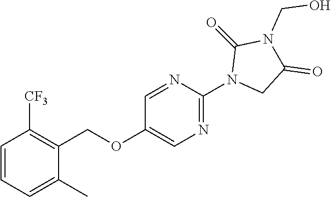 [SMILES: Cc1cccc(C(F)(F)F)c1COc1cnc(N2CC(=O)N(CO)C2=O)nc1] | 1H NMR (399 MHz, DMSO) δ 8.67 (s, 2H), 7.70- 7.62 (m, 2H), 7.56 (t, J = 7.7 Hz, 1H), 6.46 (t, J = 7.1 Hz, 1H), 5.30 (s, 2H), 4.86 (d, J = 7.1 Hz, 2H), 4.56 (s, 2H), 2.45 (s, 3H) | 367 |
| 242 | 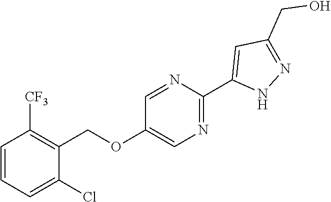 [SMILES: OCc1cc(-c2ncc(OCc3c(Cl)cccc3C(F)(F)F)cn2)[nH]n1] | 1H NMR (399 MHz, DMSO) δ 8.77 (s, 2H), 7.98 (d, J = 8.1 Hz, 1H), 7.89 (d, J = 7.9 Hz, 1H), 7.75 (t, J = 8.0 Hz, 1H), 6.79 (s, 1H), 5.45 (s, 2H), 4.50 (s, 2H). | 385 |
| 243 | 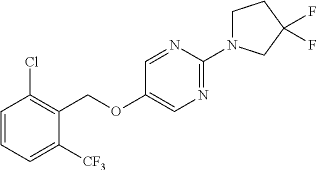 [SMILES: FC1(F)CCN(c2ncc(OCc3c(Cl)cccc3C(F)(F)F)cn2)C1] | 1H NMR (399 MHz, DMSO) δ 8.36 (s, 2H), 7.92 (d, J = 7.9 Hz, 1H), 7.84 (d, J = 7.5 Hz, 1H), 7.69 (td, J = 8.0, 1.0 Hz, 2H), 5.22 (s, 2H), 3.86 (t, J = 13.3 Hz, 2H), 3.68 (t, J = 7.3 Hz, 2H), 2.60-2.45 (m, 2H). | 394 |
| 244 | 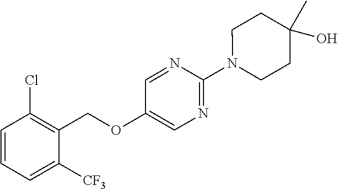 [SMILES: CC1(O)CCN(c2ncc(OCc3c(Cl)cccc3C(F)(F)F)cn2)CC1] | 1H NMR (399 MHz, DMSO) δ 8.27 (s, 2H), 7.92 (d, J = 8.2 Hz, 1H), 7.84 (d, J = 8.1 Hz, 1H), 7.68 (t, J = 8.0 Hz, 1H), 5.19 (s, 2H), 4.34 (s, 1H), 4.03 (dt, J = 13.3, 4.4 Hz, 2H), 3.39 (ddd, J = 13.5, 10.3, 3.5 Hz, 2H), 1.53-1.36 (m, 4H), 1.14 (s, 3H). | 402 |
| 245 | 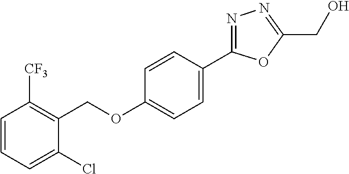 [SMILES: OCc1nnc(-c2ccc(OCc3c(Cl)cccc3C(F)(F)F)cc2)o1] | 1H NMR (399 MHz, DMSO) δ 8.02-7.93 (m, 3H), 7.87 (dd, J = 8.0, 1.2 Hz, 1H), 7.72 (t, J = 8.0 Hz, 1H), 7.34-7.26 (m, 2H), 5.94 (t, J = 6.2 Hz, 1H), 5.31 (s, 2H), 4.71 (d, J = 6.2 Hz, 2H) | 385 |
| 246 | 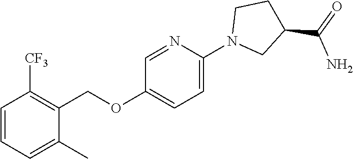 [SMILES: Cc1cccc(C(F)(F)F)c1COc1ccc(N2CC[C@@H](C(N)=O)C2)nc1] | 1H NMR (399 MHz, DMSO) δ 8.28 (s, 2H), 7.67- 7.59 (m, 2H), 7.57-7.49 (m, 1H), 7.49-7.44 (m, 1H), 6.93 (s, 1H), 5.11 (d, J = 1.3 Hz, 2H), 3.67 (dd, J = 10.8, 7.9 Hz, 1H), 3.58 (ddd, J = 10.6, 7.9, 4.7 Hz, 1H), 3.53- 3.36 (m, 2H), 3.02 (p, J = 7.6 Hz, 1H), 2.46 (s, 3H), 2.19-1.99 (m, 2H). | 381 |
| 247 | 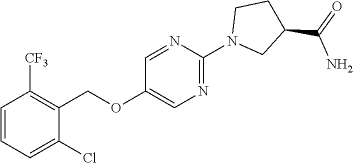 [SMILES: NC(=O)[C@@H]1CCN(c2ncc(OCc3c(Cl)cccc3C(F)(F)F)cn2)C1] | 1H NMR (399 MHz, DMSO) δ 8.28 (s, 2H), 7.93 (dd, J = 8.1, 1.2 Hz, 1H), 7.84 (dd, J = 8.0, 1.2 Hz, 1H), 7.69 (td, J = 8.0, 0.9 Hz, 1H), 7.46 (s, 1H), 6.93 (s, 1H), 5.19 (d, J = 1.3 Hz, 2H), 3.67 (dd, J = 10.8, 7.9 Hz, 1H), 3.58 (ddd, J = 10.5, 7.9, 4.7 Hz, 1H), 3.53-3.36 (m, 2H), 3.02 (p, J = 7.5 Hz, 1H), | 401 |
| | | 2.19-1.99 (m, 2H). | |
| 248 | 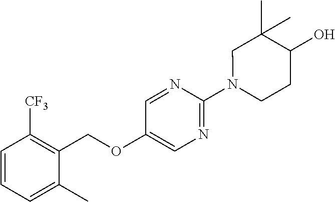 [SMILES: Cc1cccc(C(F)(F)F)c1COc1cnc(N2CCC(O)C(C)(C)C2)nc1] | 1H NMR (399 MHz, DMSO) δ 8.23 (s, 2H), 7.66- 7.58 (m, 2H), 7.52 (t, J = 7.7 Hz, 1H), 5.10 (s, 2H), 4.63 (d, J = 4.7 Hz, 1H), 4.24 (dtd, J = 13.4, 4.9, 1.8 Hz, 1H), 3.98 (dd, J = 13.0, 1.7 Hz, 1H), 3.32-3.26 (m, 1H), 3.15 (ddd, J = 13.4, 10.3, 3.5 Hz, 1H), 2.88 (d, J = 13.0 Hz, 1H), 2.44 (s, 3H), 1.66 (dq, J = 12.8, 4.2 Hz, 1H), 1.46 (ddt, J = 17.6, 9.9, | 396 |
| | | 4.4 Hz, 1H), 0.90 (s, 3H), | |
| | | 0.76 (s, 3H) | |
| 249 | 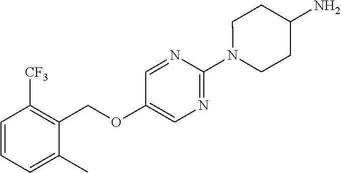 [SMILES: Cc1cccc(C(F)(F)F)c1COc1cnc(N2CCC(N)CC2)nc1] | 1H NMR (399 MHz, DMSO) δ 8.38 (s, 1H), 8.30 (s, 2H), 7.67-7.59 (m, 2H), 7.53 (t, J = 7.7 Hz, 1H), 5.12 (s, 2H), 4.49 (dt, J = 13.3, 3.6 Hz, 2H), 3.37 (br. s, 4H), 3.09 (tt, J = 10.8, 4.0 Hz, 1H), 2.93 (ddd, J = 13.5, 11.9, 2.6 Hz, 2H), 2.44 (s, 3H), 1.85 (dd, J = 12.4, 3.0 Hz, 2H), 1.32 (qd, J = 12.2, | 367 |
| | | 4.1 Hz, 2H) | |
| 250 | 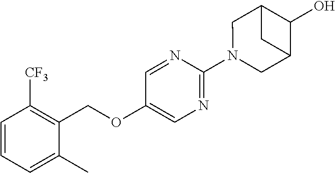 [SMILES: Cc1cccc(C(F)(F)F)c1COc1cnc(N2CC3CC(C2)C3O)nc1] | 1H NMR (399 MHz, DMSO) δ 8.30 (s, 2H), 7.67- 7.59 (m, 2H), 7.52 (t, J = 7.8 Hz, 1H), 5.20 (d, J = 4.2 Hz, 1H), 5.11 (s, 2H), 4.00 (td, J = 5.8, 4.1 Hz, 1H), 3.68 (dt, J = 11.7, 1.5 Hz, 2H), 3.54 (d, J = 11.6 Hz, 2H), 2.55-2.51 (m, 2H), 2.46 (s, 3H), 1.57 (dt, J = 10.9, 5.9 Hz, 1H), 1.31 (d, J = 9.6 Hz, 1H) | 380 |
| 251 | 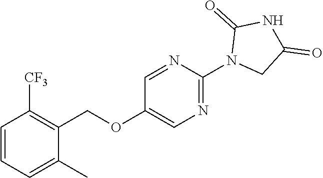 [SMILES: Cc1cccc(C(F)(F)F)c1COc1cnc(N2CC(=O)NC2=O)nc1] | 1H NMR (399 MHz, DMSO) δ 11.21 (s, 1H), 8.62 (s, 2H), 7.69-7.61 (m, 2H), 7.59-7.51 (m, 1H), 5.27 (d, J = 1.2 Hz, 2H), 4.47 (s, 2H), 2.44 (s, 3H) | 367 |
| 252 | 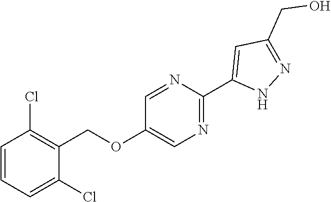 [SMILES: OCc1cc(-c2ncc(OCc3c(Cl)cccc3Cl)cn2)[nH]n1] | 1H NMR (399 MHz, DMSO) δ 13.38 (s, 1H), 8.79 (s, 2H), 7.64 (dd, J = 8.1, 0.9 Hz, 2H), 7.55 (dd, J = 8.9, 7.1 Hz, 1H), 6.82 (s, 1H), 5.52 (s, 2H), 5.10 (s, 1H), 4.52 (s, 2H) | 351 |
| 253 | 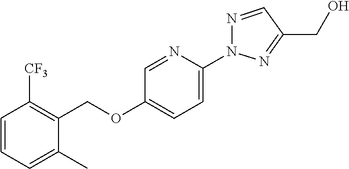 [SMILES: Cc1cccc(C(F)(F)F)c1COc1ccc(-n2ncc(CO)n2)nc1] | 1H NMR (399 MHz, DMSO) δ 8.38 (d, J = 2.8 Hz, 1H), 8.02 (s, 1H), 7.95 (d, J = 9.0 Hz, 1H), 7.82 (dd, J = 9.0, 3.0 Hz, 1H), 7.67 (t, J = 7.6 Hz, 2H), 7.57 (t, J = 7.8 Hz, 1H), 5.44 (t, J = 5.8 Hz, 1H), 5.29 (d, J = 1.3 Hz, 2H), 4.66 (d, J = 5.8 Hz, 2H), 3.30 (s, 1H), 2.46 (s, 3H) | 365 |
| 254 | 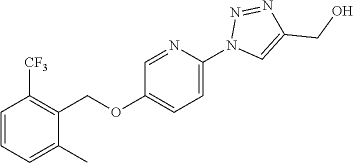 [SMILES: Cc1cccc(C(F)(F)F)c1COc1ccc(-n2cc(CO)nn2)nc1] | 1H NMR (399 MHz, DMSO) δ 8.58 (s, 1H), 8.42 (d, J = 2.9 Hz, 1H), 8.09 (d, J = 9.0 Hz, 1H), 7.89 (dd, J = 9.0, 3.0 Hz, 1H), 7.67 (t, J = 7.7 Hz, 2H), 7.57 (t, J = 7.8 Hz, 1H), 5.36-5.27 (m, 3H), 4.63 (d, J = 5.6 Hz, 2H), 3.30 (s, 1H), 2.46 (s, 3H) | 365 |
| 255 | 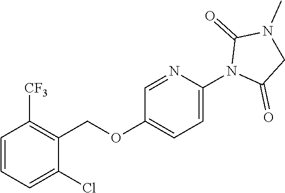 [SMILES: CN1CC(=O)N(c2ccc(OCc3c(Cl)cccc3C(F)(F)F)cn2)C1=O] | 1H NMR (399 MHz, DMSO) δ 8.39 (d, J = 3.0 Hz, 1H), 7.96 (dd, J = 8.2, 1.2 Hz, 1H), 7.87 (dd, J = 8.0, 1.2 Hz, 1H), 7.80-7.67 (m, 2H), 7.39 (d, J = 8.7 Hz, 1H), 5.34 (d, J = 1.3 Hz, 2H), 4.15 (s, 2H), 2.93 (s, 3H) | 400 |
| 256 | 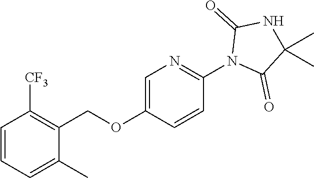 [SMILES: Cc1cccc(C(F)(F)F)c1COc1ccc(N2C(=O)NC(C)(C)C2=O)nc1] | 1H NMR (399 MHz, DMSO) δ 8.54 (s, 1H), 8.37 (dd, J = 3.1, 0.5 Hz, 1H), 7.75 (dd, J = 8.7, 3.1 Hz, 1H), 7.71-7.62 (m, 2H), 7.61-7.52 (m, 1H), 7.44 (dd, J = 8.6, 0.6 Hz, 1H), 5.26 (d, J = 1.3 Hz, 2H), 2.45 (s, 3H), 1.41 (s, 6H) | 394 |
| 257 | 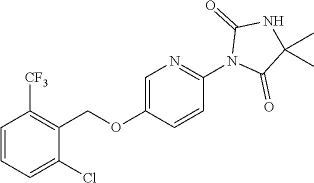 [SMILES: CC1(C)NC(=O)N(c2ccc(OCc3c(Cl)cccc3C(F)(F)F)cn2)C1=O] | 1H NMR (399 MHz, DMSO) δ 8.55 (s, 1H), 8.37 (d, J = 3.1 Hz, 1H), 7.95 (dd, J = 8.1, 1.2 Hz, 1H), 7.87 (dd, J = 8.0, 1.2 Hz, 1H), 7.80-7.67 (m, 2H), 7.44 (d, J = 8.7 Hz, 1H), 5.36-5.31 (m, 2H), 1.41 (s, 6H) | 414 |
| 258 | 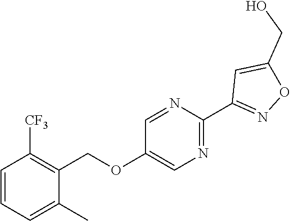 [SMILES: Cc1cccc(C(F)(F)F)c1COc1cnc(-c2cc(CO)on2)nc1] | 1H NMR (399 MHz, DMSO) δ 8.90 (s, 2H), 8.43 (s, 0H), 7.71 (t, J = 7.8 Hz, 2H), 7.62 (t, J = 7.8 Hz, 1H), 6.91 (s, 1H), 5.80 (s, 1H), 5.44 (s, 2H), 4.69 (s, 2H), 2.50 (s, 3H), 1.28 (s, 1H) | 366 |
| 259 | 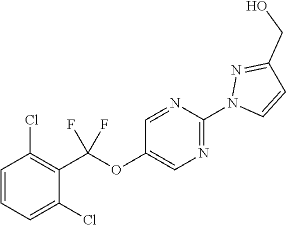 [SMILES: OCc1ccn(-c2ncc(OC(F)(F)c3c(Cl)cccc3Cl)cn2)n1] | 1H NMR (400 MHz, DMSO) δ 8.92 (s, 2H), 8.56 (d, J = 2.8 Hz, 1H), 7.71 (d, J = 7.2 Hz, 2H), 7.64 (dd, J = 8.8, 6.8 Hz, 1H), 6.58 (d, J = 2.8 Hz, 1H), 4.53 (s, 2H). | 387 |
| 260 | 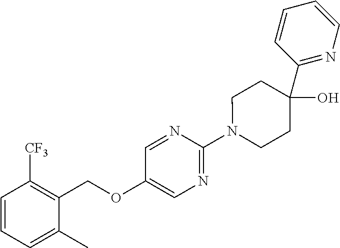 [SMILES: Cc1cccc(C(F)(F)F)c1COc1cnc(N2CCC(O)(c3ccccn3)CC2)nc1] | 1H NMR (400 MHz, DMSO) δ 8.50-8.45 (m, 1H), 8.31 (s, 2H), 7.79 (td, J = 7.7, 1.8 Hz, 1H), 7.70 (d, J = 8.0 Hz, 1H), 7.65-7.62 (m, 2H), 7.53 (t, J = 7.8 Hz, 1H), 7.23 (m, J = 7.4, 4.8, 1.2 Hz, 1H), 5.35 (s, 1H), 5.14 (s, 2H), 4.46 (d, J = 12.6 Hz, 2H), 3.36-3.35 (m, 1H), 3.29-3.28 (m, 1H), 2.47 (s, 3H), 2.09 (td, J = 13.4, 4.6 Hz, 2H), 1.55 (d, J = 12.5 Hz, 2H). | 445 |
| 261 | 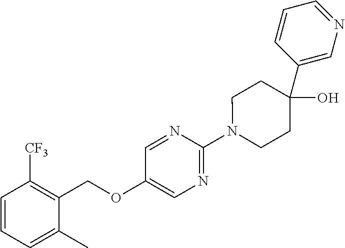 [SMILES: Cc1cccc(C(F)(F)F)c1COc1cnc(N2CCC(O)(c3cccnc3)CC2)nc1] | 1H NMR (400 MHz, DMSO) δ 8.71 (d, J = 1.9 Hz, 1H), 8.44 (dd, J = 4.7, 1.5 Hz, 1H), 8.31 (s, 2H), 7.89- 7.84 (m, 1H), 7.65-7.62 (m, 2H), 7.53 (t, J = 7.6 Hz, 1H), 7.33 (dd, J = 7.4, 4.7 Hz, 1H), 5.31 (s, 1H), 5.13 (s, 2H), 4.48 (d, J = 12.8 Hz, 2H), 3.30-3.26 (m, 2H), 2.46 (s, 3H), 1.92 (td, J = 13.0, 4.4 Hz, 2H), 1.71 (d, J = 12.4 Hz, 2H). | 445 |
| 262 | 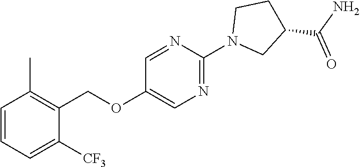 [SMILES: Cc1cccc(C(F)(F)F)c1COc1cnc(N2CC[C@H](C(N)=O)C2)nc1] | 1H NMR (399 MHz, DMSO) δ 8.28 (s, 2H), 7.67- 7.59 (m, 2H), 7.57-7.49 (m, 1H), 7.46 (s, 1H), 6.93 (s, 1H), 5.11 (d, J = 1.3 Hz, 2H), 3.67 (dd, J = 10.8, 7.9 Hz, 1H), 3.58 (ddd, J = 10.6, 7.9, 4.7 Hz, 1H), 3.53-3.36 (m, 2H), 3.02 (p, J = 7.5 Hz, 1H), 2.46 (s, 3H), 2.19-1.97 (m, 2H) | 381 |
| 263 | 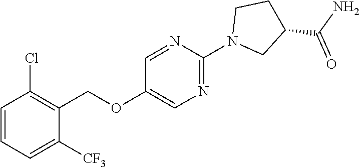 [SMILES: NC(=O)[C@H]1CCN(c2ncc(OCc3c(Cl)cccc3C(F)(F)F)cn2)C1] | 1H NMR (399 MHz, DMSO) δ 8.28 (s, 2H), 7.93 (dd, J = 8.2, 1.2 Hz, 1H), 7.84 (dd, J = 8.0, 1.2 Hz, 1H), 7.69 (td, J = 8.0, 0.9 Hz, 1H), 7.46 (s, 1H), 6.93 (s, 1H), 5.19 (d, J = 1.3 Hz, 2H), 3.67 (dd, J = 10.8, 7.9 Hz, 1H), 3.58 (ddd, J = 10.6, 7.9, 4.7 Hz, 1H), 3.53-3.36 (m, 2H), 3.02 (p, J = 7.6 Hz, 1H), | 401 |
| | | 2.19-1.97 (m, 2H) | |
| 264 | 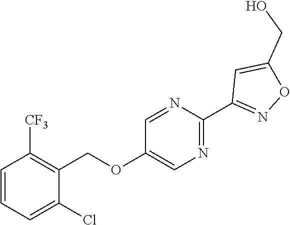 [SMILES: OCc1cc(-c2ncc(OCc3c(Cl)cccc3C(F)(F)F)cn2)no1] | 1H NMR (399 MHz, DMSO) δ 8.89 (s, 2H), 7.98 (dd, J = 8.1, 1.2 Hz, 1H), 7.89 (dd, J = 8.0, 1.2 Hz, 1H), 7.74 (td, J = 8.1, 0.9 Hz, 1H), 6.87 (s, 1H), 5.79-5.70 (m, 1H), 5.49 (d, J = 1.2 Hz, 2H), 4.65 (dd, J = 6.0, 0.8 Hz, 2H) | 386 |
| 265 | 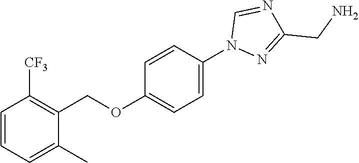 [SMILES: Cc1cccc(C(F)(F)F)c1COc1ccc(-n2cnc(CN)n2)cc1] | 1H NMR (400 MHz, DMSO) δ 9.08 (s, 1H), 7.77 (d, J = 8.9 Hz, 2H), 7.65 (t, J = 8.0 Hz, 2H), 7.54 (t, J = 7.7 Hz, 1H), 7.23 (d, J = 9.0 Hz, 2H), 5.18 (s, 2H), 3.78 (s, 2H), 2.44 (s, 3H), 2.05 (d, J = 59.5 Hz, 2H). | 363 |
| 266 | 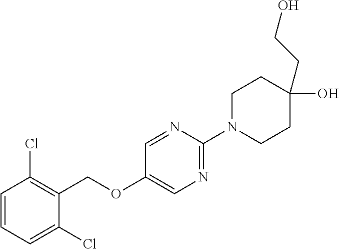 [SMILES: OCCC1(O)CCN(c2ncc(OCc3c(Cl)cccc3Cl)cn2)CC1] | 1H NMR (400 MHz, DMSO) δ 8.24 (s, 2H), 7.59- 7.52 (m, 2H), 7.46 (dd, J = 9.0, 7.1 Hz, 1H), 5.24 (s, 2H), 4.13-4.05 (m, 2H), 3.57 (d, J = 7.1 Hz, 2H), 3.32 (m, J = 13.5, 10.6, 3.5 Hz, 2H), 1.61 (t, J = 7.1 Hz, 2H), 1.56-1.39 (m, 4H). | 398 |
| 267 | 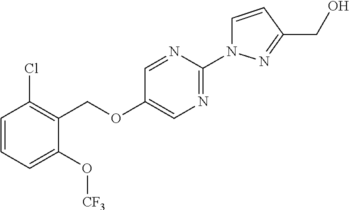 [SMILES: OCc1ccn(-c2ncc(OCc3c(Cl)cccc3OC(F)(F)F)cn2)n1] | 1H NMR (400 MHz, DMSO) δ 8.73 (d, J = 5.8 Hz, 2H), 8.49 (d, J = 2.5 Hz, 1H), 7.65 (dd, J = 12.5, 5.0 Hz, 2H), 7.52 (d, J = 7.5 Hz, 1H), 6.52 (d, J = 2.5 Hz, 1H), 5.40 (s, 2H), 5.24 (t, J = 5.2 Hz, 1H), 4.52 (d, J = 5.9 Hz, 2H). | 401 |
| 268 | 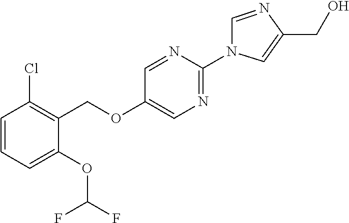 [SMILES: OCc1cn(-c2ncc(OCc3c(Cl)cccc3OC(F)F)cn2)cn1] | 1H NMR (400 MHz, DMSO) δ 8.71 (s, 2H), 8.49 (d, J = 2.4 Hz, 1H), 7.56 (d, J = 8.0 Hz, 1H), 7.52- 7.48 (m, 1H), 7.33 (t, J = 80.8 Hz, 1H), 7.31 (s, 1H), 6.52 (d, J = 2.8 Hz, 1H), 5.36 (s, 2H), 5.23 (t, J = 6.0 Hz, 1H), 4.52 (d, J = 6.0 Hz, 2H). | 383 |
| 269 | 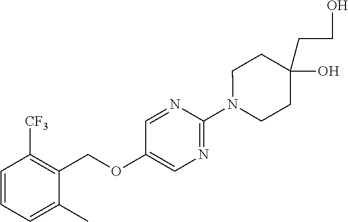 [SMILES: Cc1cccc(C(F)(F)F)c1COc1cnc(N2CCC(O)(CCO)CC2)nc1] | 1H NMR (400 MHz, DMSO) δ 8.26 (s, 2H), 7.63 (t, J = 6.5 Hz, 2H), 7.52 (t, J = 7.7 Hz, 1H), 5.11 (s, 2H), 4.41-4.28 (m, 2H), 4.11 (d, J = 12.8 Hz, 2H), 3.58 (td, J = 7.0, 4.7 Hz, 2H), 3.29 (d, J = 3.6 Hz, 1H), 3.32-3.40 (m, 1H), 2.45 (s, 3H), 1.60 (t, J = 7.1 Hz, 2H), 1.55-1.37 (m, 4H). | 412 |
| 270 | 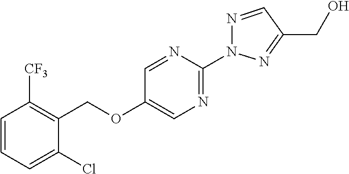 [SMILES: OCc1cnn(-c2ncc(OCc3c(Cl)cccc3C(F)(F)F)cn2)n1] | 1H NMR (399 MHz, DMSO) δ 8.87 (s, 2H), 8.08 (s, 1H), 8.01-7.95 (m, 1H), 7.89 (dd, J = 7.6, 1.0 Hz, 1H), 7.75 (td, J = 8.0, 0.9 Hz, 1H), 5.51-5.44 (m, 3H), 4.66 (d, J = 5.8 Hz, 2H) | 386 |
| 271 | 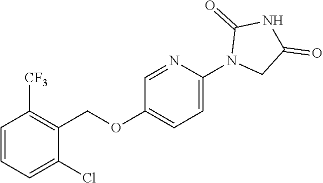 [SMILES: O=C1CN(c2ccc(OCc3c(Cl)cccc3C(F)(F)F)cn2)C(=O)N1] | 1H NMR (399 MHz, DMSO) δ 11.25 (s, 1H), 8.20 (dd, J = 3.1, 0.7 Hz, 1H), 8.10 (dd, J = 9.1, 0.6 Hz, 1H), 7.94 (dd, J = 8.2, 1.2 Hz, 1H), 7.86 (dd, J = 8.0, 1.2 Hz, 1H), 7.75-7.62 (m, 2H), 5.27 (d, J = 1.3 Hz, 2H), 4.45 (s, 2H) | 386 |
| 272 | 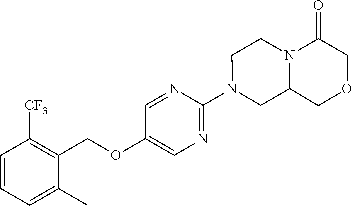 [SMILES: Cc1cccc(C(F)(F)F)c1COc1cnc(N2CCN3C(=O)COCC3C2)nc1] | 1H NMR (399 MHz, DMSO) δ 8.35 (s, 2H), 7.66- 7.60 (m, 2H), 7.53 (t, J = 7.8 Hz, 1H), 5.15 (s, 2H), 4.60-4.48 (m, 2H), 4.47- 4.40 (m, 1H), 4.13-4.02 (m, 1H), 4.07 (s, 2H), 3.67-3.54 (m, 2H), 2.86 (td, J = 12.5, 3.1 Hz, 1H), 2.78-2.69 (m, 2H), 2.44 (s, 3H) | 423 |
| 273 | 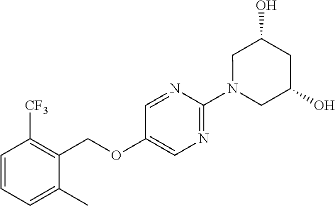 [SMILES: Cc1cccc(C(F)(F)F)c1COc1cnc(N2C[C@H](O)C[C@H](O)C2)nc1] | 1H NMR (399 MHz, DMSO) δ 8.27 (s, 2H), 7.66- 7.59 (m, 2H), 7.52 (t, J = 7.7 Hz, 1H), 5.12 (s, 2H), 4.96 (d, J = 4.7 Hz, 2H), 4.59 (dd, J = 12.5, 4.7 Hz, 2H), 3.46-3.35 (m, 2H), 2.44 (s, 3H), 2.48-2.39 (m, 2H), 2.21-2.11 (m, 1H), 1.24 (q, J = 11.1 Hz, 1H) | 384 |
| 274 | 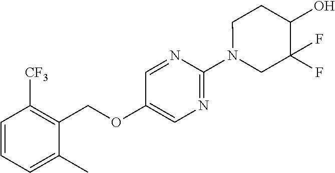 [SMILES: Cc1cccc(C(F)(F)F)c1COc1cnc(N2CCC(O)C(F)(F)C2)nc1] | 1H NMR (399 MHz, DMSO) δ 8.33 (s, 2H), 7.67- 7.60 (m, 2H), 7.53 (t, J = 7.7 Hz, 1H), 5.71 (d, J = 5.4 Hz, 1H), 5.14 (s, 2H), 4.20 (ddd, J = 18.3, 13.8, 6.3 Hz, 1H), 3.99-3.81 (m, 3H), 3.68-3.59 (m, 1H), 2.45 (s, 3H), 1.84 (dq, J = 16.9, 3.6 Hz, 1H), 1.73-1.59 (m, 1H) | 404 |
| 275 | 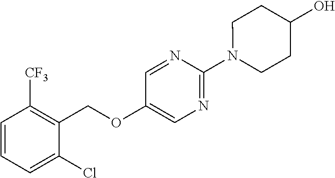 [SMILES: OC1CCN(c2ncc(OCc3c(Cl)cccc3C(F)(F)F)cn2)CC1] | 1H NMR (399 MHz, DMSO) δ 8.29 (s, 2H), 7.96- 7.90 (m, 1H), 7.84 (dd, J = 8.0, 1.2 Hz, 1H), 7.69 (td, J = 8.0, 0.9 Hz, 1H), 5.20 (d, J = 1.3 Hz, 2H), 4.69 (d, J = 4.3 Hz, 1H), 4.24-4.14 (m, 2H), 3.70 (tt, J = 8.4, 4.2 Hz, 1H), 3.19 (ddd, J = 13.3, 10.0, 3.2 Hz, 2H), 1.76 (dddd, J = 11.8, 8.2, 5.2, 3.3 Hz, 2H), 1.38-1.30 (m, 1H), | 388 |
| | | 1.29 (dt, J = 8.8, 3.8 Hz, 1H) | |
| 276 | 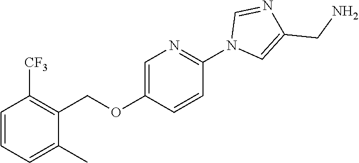 [SMILES: Cc1cccc(C(F)(F)F)c1COc1ccc(-n2cnc(CN)c2)nc1] | 1H NMR (400 MHz, DMSO) δ 8.47 (s, 1H), 8.42 (s, 1H), 8.33 (s, 1H), 7.90 (s, 1H), 7.80 (s, 2H), 7.66 (t, J = 7.9 Hz, 2H), 7.56 (t, J = 7.6 Hz, 1H), 5.26 (s, 2H), 3.90 (s, 2H), 2.45 (s, 3H). | 363 |
| 277 | 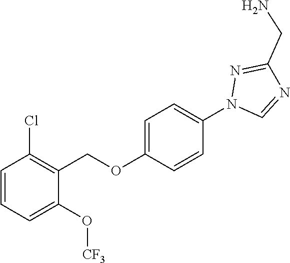 [SMILES: NCc1ncn(-c2ccc(OCc3c(Cl)cccc3OC(F)(F)F)cc2)n1] | 1H NMR (400 MHz, DMSO-d6) δ 9.10 (s, 1H), 7.77 (d, J = 8.9 Hz, 2H), 7.69- 7.58 (m, 2H), 7.50 (d, J = 7.8 Hz, 1H), 7.26-7.19 (m, 2H), 5.34 (s, 1H), 5.22 (s, 2H), 4.54-4.49 (m, 2H), 1.24 (s, 1H). | 399 |
| 278 | 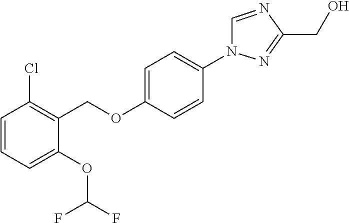 [SMILES: OCc1ncn(-c2ccc(OCc3c(Cl)cccc3OC(F)F)cc2)n1] | 1H NMR (400 MHz, DMSO-d6) δ 9.09 (s, 1H), 7.76 (d, J = 9.0 Hz, 2H), 7.61- 7.44 (m, 2H), 7.31 (d, J = 8.8 Hz, 1H), 7.27-7.17 (m, 2H), 7.16-7.13 (d, J = 12 Hz, 1H), 5.35 (t, J = 6.0 Hz, 1H), 5.19 (s, 2H), 4.51 (d, J = 6.1 Hz, 2H). | 382 |
| 279 | 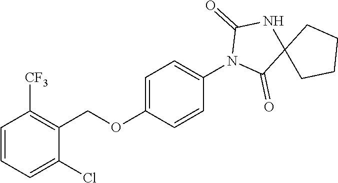 [SMILES: O=C1NC2(CCCC2)C(=O)N1c1ccc(OCc2c(Cl)cccc2C(F)(F)F)cc1] | 1H NMR (399 MHz, DMSO) δ 8.70 (s, 1H), 7.94 (dd, J = 8.2, 1.2 Hz, 1H), 7.85 (dd, J = 8.0, 1.2 Hz, 1H), 7.70 (td, J = 8.0, 0.9 Hz, 1H), 7.34-7.26 (m, 2H), 7.19-7.10 (m, 2H), 5.25- 5.21 (m, 2H), 2.15-1.97 (m, 2H), 1.91-1.70 (m, 6H) | 439 |
| 280 | 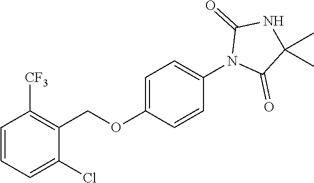 [SMILES: CC1(C)NC(=O)N(c2ccc(OCc3c(Cl)cccc3C(F)(F)F)cc2)C1=O] | 1H NMR (399 MHz, DMSO) δ 8.50 (s, 1H), 7.95 (dd, J = 8.3, 1.2 Hz, 1H), 7.86 (dd, J = 8.0, 1.2 Hz, 1H), 7.71 (td, J = 8.0, 1.0 Hz, 1H), 7.35-7.26 (m, 2H), 7.20-7.11 (m, 2H), 5.26- 5.21 (m, 2H), 1.40 (s, 6H) | 413 |
| 281 | 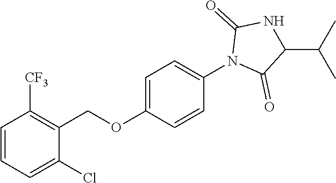 [SMILES: CC(C)C1NC(=O)N(c2ccc(OCc3c(Cl)cccc3C(F)(F)F)cc2)C1=O] | 1H NMR (399 MHz, DMSO) δ 8.48 (d, J = 1.4 Hz, 1H), 7.95 (dd, J = 8.2, 1.2 Hz, 1H), 7.86 (dd, J = 8.0, 1.2 Hz, 1H), 7.71 (td, J = 8.0, 0.9 Hz, 1H), 7.30-7.21 (m, 2H), 7.20-7.12 (m, 2H), 5.24 (d, J = 1.3 Hz, 2H), 4.13 (dd, J = 3.5, 1.3 Hz, 1H), 2.13 (ddh, J = 10.3, 7.0, 3.5 Hz, 1H), 1.03 (d, J = 6.9 Hz, 3H), 0.89 (d, J = 6.8 Hz, 3H) | 427 |
| 282 | 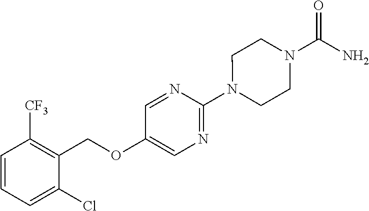 [SMILES: NC(=O)N1CCN(c2ncc(OCc3c(Cl)cccc3C(F)(F)F)cn2)CC1] | 1H NMR (399 MHz, DMSO) δ 8.26 (s, 2H), 7.86 (d, J = 8.0 Hz, 1H), 7.80- 7.74 (m, 1H), 7.62 (t, J = 8.0 Hz, 1H), 5.97 (s, 2H), 5.15 (s, 2H), 3.58-3.51 (m, 4H), 3.33-3.26 (m, 4H) | 416 |
| 283 | 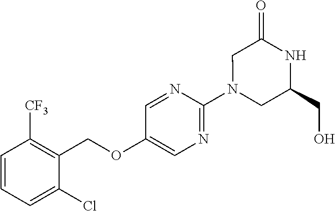 [SMILES: O=C1CN(c2ncc(OCc3c(Cl)cccc3C(F)(F)F)cn2)C[C@H](CO)N1] | 1H NMR (399 MHz, DMSO) δ 8.30 (s, 2H), 7.94- 7.89 (m, 1H), 7.86 (dd, J = 8.1, 1.2 Hz, 1H), 7.77 (dd, J = 8.0, 1.2 Hz, 1H), 7.62 (td, J = 8.0, 0.9 Hz, 1H), 5.18- 5.13 (m, 2H), 4.84 (t, J = 5.1 Hz, 1H), 4.06 (s, 1H), 4.00- 3.90 (m, 2H), 3.65 (dd, J = 13.1, 6.3 Hz, 1H), 3.42-3.32 (m, 2H) | 417 |
| 284 | 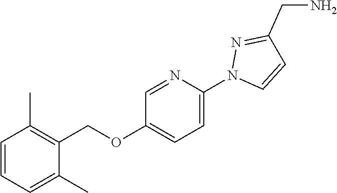 [SMILES: Cc1cccc(C)c1COc1ccc(-n2ccc(CN)n2)nc1] | 1H NMR (400 MHz, Chloroform) δ 8.34 (s, 2H), 8.09 (d, J = 2.6 Hz, 1H), 7.81 (d, J = 9.0 Hz, 1H), 7.38 (d, J = 8.9 Hz, 1H), 7.22-7.14 (m, 1H), 7.08 (d, J = 7.5 Hz, 2H), 6.49 (s, 1H), 5.05 (s, 3H), 4.17 (s, 2H), 2.38 (s, 6H). | 309 |
| 285 | 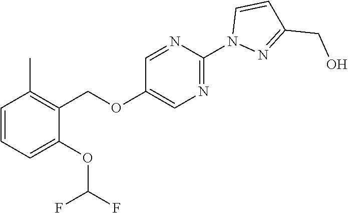 [SMILES: Cc1cccc(OC(F)F)c1COc1cnc(-n2ccc(CO)n2)nc1] | 1H NMR (400 MHz, DMSO) δ 8.69 (s, 2H), 8.48 (d, J = 2.5 Hz, 1H), 7.40 (dd, J = 9.6, 6.3 Hz, 1H), 7.23- 7.18 (m, 1H), 7.12 (d, J = 8.3 Hz, 1H), 6.52 (d, J = 2.5 Hz, 1H), 5.31 (s, 2H), 5.23 (t, J = 5.7 Hz, 1H), 4.52 (d, J = 5.5 Hz, 2H), 2.41 (s, 3H). | 363 |
| 286 | 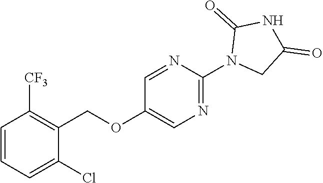 [SMILES: O=C1CN(c2ncc(OCc3c(Cl)cccc3C(F)(F)F)cn2)C(=O)N1] | 1H NMR (399 MHz, DMSO) δ 11.22 (s, 1H), 8.64 (s, 2H), 7.96 (dd, J = 8.1, 1.2 Hz, 1H), 7.87 (dd, J = 8.1, 1.2 Hz, 1H), 7.72 (td, J = 8.1, 1.0 Hz, 1H), 5.36 (d, J = 1.2 Hz, 2H), 4.47 (s, 2H) | 387 |
| 287 | 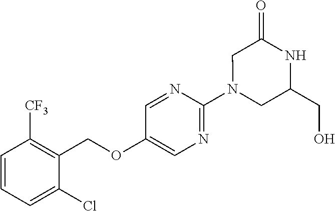 [SMILES: O=C1CN(c2ncc(OCc3c(Cl)cccc3C(F)(F)F)cn2)CC(CO)N1] | 1H NMR (399 MHz, DMSO) δ 8.37 (s, 2H), 8.01- 7.96 (m, 1H), 7.93 (dd, J = 8.2, 1.2 Hz, 1H), 7.84 (dd, J = 8.0, 1.2 Hz, 1H), 7.74- 7.65 (m, 1H), 5.23 (d, J = 1.3 Hz, 2H), 4.92 (s, 1H), 4.13 (s, 1H), 4.07-3.97 (m, 2H), 3.77-3.67 (m, 1H), 3.44 (q, J = 9.9, 7.2 Hz, 3H) | 417 |
| 288 | 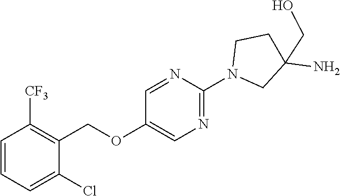 [SMILES: NC1(CO)CCN(c2ncc(OCc3c(Cl)cccc3C(F)(F)F)cn2)C1] | 1H NMR (399 MHz, DMSO) δ 8.29 (s, 1H), 8.21 (s, 2H), 7.85 (dd, J = 8.1, 1.2 Hz, 1H), 7.77 (dd, J = 8.0, 1.2 Hz, 1H), 7.66-7.57 (m, 1H), 5.14-5.09 (m, 2H), 3.59-3.38 (m, 3H), 3.27 (d, J = 11.4 Hz, 1H), 1.96 (dt, J = 12.6, 8.1 Hz, 1H), 1.75 (ddd, J = 12.5, 7.5, 4.7 Hz, 1H) | 403 |
| 289 | 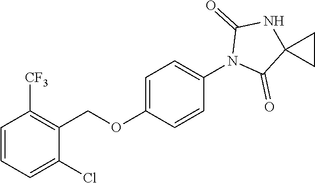 [SMILES: O=C1NC2(CC2)C(=O)N1c1ccc(OCc2c(Cl)cccc2C(F)(F)F)cc1] | 1H NMR (399 MHz, DMSO) δ 8.59 (s, 1H), 7.94 (dd, J = 8.1, 1.2 Hz, 1H), 7.85 (dd, J = 8.1, 1.2 Hz, 1H), 7.70 (td, J = 8.0, 0.9 Hz, 1H), 7.37-7.28 (m, 2H), 7.20-7.08 (m, 2H), 5.26- 5.19 (m, 2H), 1.41-1.26 (m, 4H) | 411 |
| 290 | 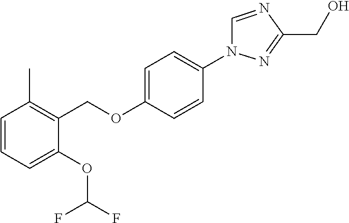 [SMILES: Cc1cccc(OC(F)F)c1COc1ccc(-n2cnc(CO)n2)cc1] | 1H NMR (400 MHz, DMSO-d6) δ 9.09 (s, 1H), 7.80-7.72 (m, 2H), 7.42- 7.34 (m, 1H), 7.25-7.16 (m, 4H), 7.11 (d, J = 8.3 Hz, 1H), 5.35 (t, J = 6.0 Hz, 1H), 5.14 (s, 2H), 4.52 (d, J = 6.0 Hz, 2H), 2.39 (s, 3H). | 362 |
| 291 | 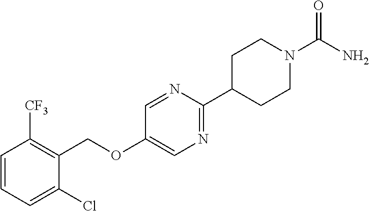 [SMILES: NC(=O)N1CCC(c2ncc(OCc3c(Cl)cccc3C(F)(F)F)cn2)CC1] | 1H NMR (400 MHz, DMSO) δ 8.64 (s, 2H), 7.95 (d, J = 8.1 Hz, 1H), 7.86 (d, J = 7.9 Hz, 1H), 7.72 (t, J = 8.0 Hz, 1H), 5.91 (s, 2H), 5.35 (s, 2H), 4.01 (d, J = 13.1 Hz, 2H), 2.97 (t, J = 11.1 Hz, 1H), 2.80 (t, J = 11.8 Hz, 2H), 1.87 (d, J = 11.2 Hz, 2H), 1.67-1.59 (m, 2H). | 415 |
| 292 | 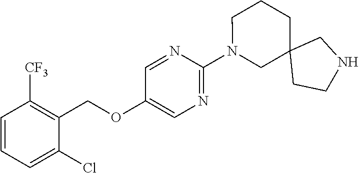 [SMILES: FC(F)(F)c1cccc(Cl)c1COc1cnc(N2CCCC3(CCNC3)C2)nc1] | 1H NMR (399 MHz, DMSO) δ 8.27 (s, 2H), 7.92 (d, J = 8.1 Hz, 1H), 7.84 (d, J = 8.0 Hz, 1H), 7.69 (t, J = 8.0 Hz, 1H), 5.20 (s, 2H), 3.79-3.70 (m, 1H), 3.71 (d, J = 12.8 Hz, 1H), 3.60-3.52 (m, 1H), 3.50 (d, J = 13.0 Hz, 1H), 3.51 (br. s, 2H), 3.09 (t, J = 7.2 Hz, 2H), 2.85 (d, J = 11.5 Hz, 1H), 2.76 (d, J = 11.5 | 427 |
| | | Hz, 1H), 1.69 (tt, J = 14.3, 6.5 | |
| | | Hz, 2H), 1.62-1.51 (m, 4H) | |
| 293 | 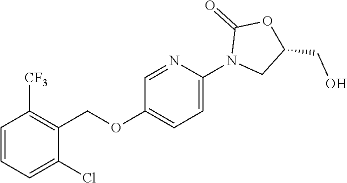 [SMILES: O=C1O[C@H](CO)CN1c1ccc(OCc2c(Cl)cccc2C(F)(F)F)cn1] | 1H NMR (399 MHz, DMSO) δ 8.28-8.22 (m, 1H), 8.09 (d, J = 9.1 Hz, 1H), 7.98 (dd, J = 8.1, 1.2 Hz, 1H), 7.89 (dd, J = 8.0, 1.2 Hz, 1H), 7.79-7.66 (m, 2H), 5.31 (d, J = 1.3 Hz, 2H), 5.24 (t, J = 5.7 Hz, 1H), 4.74 (ddt, J = 9.6, 6.2, 3.7 Hz, 1H), 4.20 (dd, J = 10.0, 9.1 Hz, 1H), 4.00 (dd, J = 10.0, 6.3 Hz, 1H), 3.73 (ddd, J = | 403 |
| | | 12.3, 5.5, 3.3 Hz, 1H), 3.61 | |
| | | (ddd, J = 12.4, 5.8, 4.1 Hz, 1H) | |
| 294 | 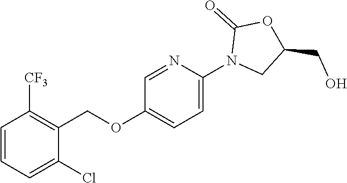 [SMILES: O=C1O[C@@H](CO)CN1c1ccc(OCc2c(Cl)cccc2C(F)(F)F)cn1] | 1H NMR (399 MHz, DMSO) δ 8.28-8.22 (m, 1H), 8.09 (d, J = 9.1 Hz, 1H), 7.98 (dd, J = 8.2, 1.2 Hz, 1H), 7.89 (dd, J = 8.0, 1.2 Hz, 1H), 7.79-7.66 (m, 2H), 5.33-5.28 (m, 2H), 5.24 (t, J = 5.6 Hz, 1H), 4.80- 4.69 (m, 1H), 4.20 (dd, J = 10.0, 9.1 Hz, 1H), 4.00 (dd, J = 10.0, 6.3 Hz, 1H), 3.73 (ddd, J = 12.3, 5.5, 3.3 Hz, | 403 |
| | | 1H), 3.61 (ddd, J = 12.4, 5.9, | |
| | | 4.1 Hz, 1H) | |
| 295 | 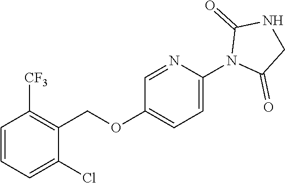 [SMILES: O=C1CNC(=O)N1c1ccc(OCc2c(Cl)cccc2C(F)(F)F)cn1] | 1H NMR (399 MHz, DMSO) δ 8.40 (d, J = 3.1 Hz, 1H), 8.30 (s, 1H), 7.97 (dd, J = 8.1, 1.2 Hz, 1H), 7.88 (dd, J = 8.0, 1.2 Hz, 1H), 7.80- 7.68 (m, 2H), 7.40 (d, J = 8.7 Hz, 1H), 5.37-5.32 (m, 2H), 4.11 (d, J = 1.1 Hz, 2H) | 386 |
| 296 | 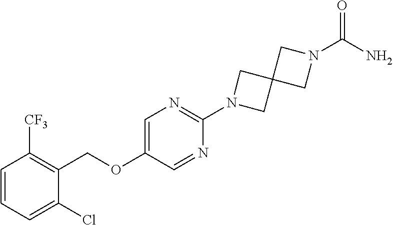 [SMILES: NC(=O)N1CC2(C1)CN(c1ncc(OCc3c(Cl)cccc3C(F)(F)F)cn1)C2] | 1H NMR (400 MHz, DMSO) δ 8.30 (s, 2H), 7.96- 7.89 (m, 1H), 7.84 (dd, J = 7.9, 1.2 Hz, 1H), 7.73-7.64 (m, 1H), 5.87 (s, 1H), 5.20 (s, 2H), 4.10 (s, 4H), 3.95 (s, 4H). | 428 |
| 297 | 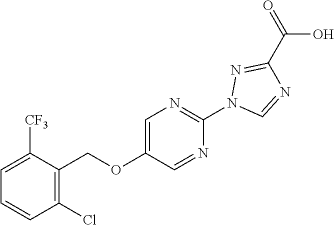 [SMILES: O=C(O)c1ncn(-c2ncc(OCc3c(Cl)cccc3C(F)(F)F)cn2)n1] | 1H NMR (400 MHz, DMSO) δ 13.63 (s, 1H), 9.43 (s, 1H), 8.91 (s, 2H), 7.98 (d, J = 8.1 Hz, 1H), 7.92-7.85 (m, 1H), 7.74 (t, J = 8.0 Hz, 1H), 5.50 (s, 2H). | 400 |
| 298 | 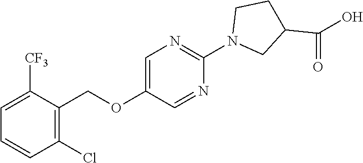 [SMILES: O=C(O)C1CCN(c2ncc(OCc3c(Cl)cccc3C(F)(F)F)cn2)C1] | 1H NMR (400 MHz, DMSO-d6) δ 12.42 (s, 1H), 8.29 (s, 2H), 7.92 (d, J = 8.1 Hz, 1H), 7.83 (d, J = 7.9 Hz, 1H), 7.68 (t, J = 8.0 Hz, 1H), 5.39-4.90 (m, 2H), 3.80- 3.56 (m, 2H), 3.59-3.42 (m, 2H), 3.24-3.04 (m, 1H), 2.31-2.08 (m, 2H). | 402 |
| 299 | 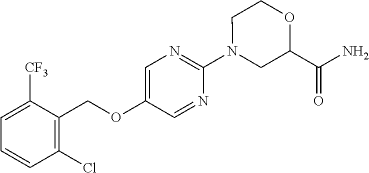 [SMILES: NC(=O)C1CN(c2ncc(OCc3c(Cl)cccc3C(F)(F)F)cn2)CCO1] | 1H NMR (400 MHz, DMSO-d6) δ 8.36 (s, 2H), 7.93 (d, J = 8.1 Hz, 1H), 7.84 (d, J = 7.9 Hz, 1H), 7.69 (t, J = 8.1 Hz, 1H), 7.34 (s, 1H), 7.27 (s, 1H), 5.23 (s, 2H), 4.64-4.53 (m, 1H), 4.25 (d, J = 13.0 Hz, 1H), 4.04-3.97 (m, 1H), 3.89 (dd, J = 10.5, 3.1 Hz, 1H), 3.59 (td, J = 11.4, 2.8 Hz, 1H), 3.00 (m, | 417 |
| | | 1H), 2.83 (dd, J = 13.2, 10.5 | |
| | | Hz, 1H). | |
| 300 | 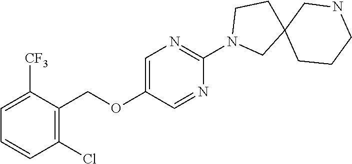 [SMILES: FC(F)(F)c1cccc(Cl)c1COc1cnc(N2CCC3(CCCNC3)C2)nc1] | 1H NMR (399 MHz, DMSO) δ 8.25 (s, 2H), 7.92 (d, J = 8.1 Hz, 1H), 7.83 (d, J = 7.8 Hz, 1H), 7.71-7.65 (m, 1H), 5.18 (s, 2H), 3.57- 3.47 (m, 2H), 3.42 (dt, J = 11.0, 7.6 Hz, 1H), 3.14 (d, J = 11.0 Hz, 1H), 2.64-2.46 (m, 4H), 1.89 (ddd, J = 12.1, 7.5, 4.5 Hz, 1H), 1.67 (dt, J = 12.4, 8.3 Hz, 1H), 1.60- | 427 |
| | | 1.51 (m, 1H), 1.50-1.36 (m, 3H). | |
| 301 | 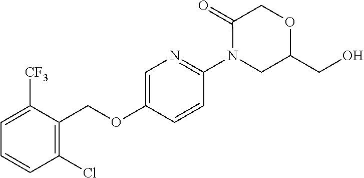 [SMILES: O=C1COC(CO)CN1c1ccc(OCc2c(Cl)cccc2C(F)(F)F)cn1] | 1H NMR (399 MHz, DMSO) δ 8.28 (d, J = 3.0 Hz, 1H), 7.95 (dd, J = 8.2, 1.2 Hz, 1H), 7.92-7.83 (m, 2H), 7.76-7.61 (m, 2H), 5.30 (d, J = 1.2 Hz, 2H), 4.98 (t, J = 5.8 Hz, 1H), 4.32 (d, J = 16.5 Hz, 1H), 4.27 (d, J = 16.6 Hz, 1H), 4.06 (dd, J = 12.5, 3.0 Hz, 1H), 3.95 (dtd, J = 10.6, 5.2, 3.0 Hz, 1H), 3.69- 3.49 (m, 3H). | 417 |
| 302 | 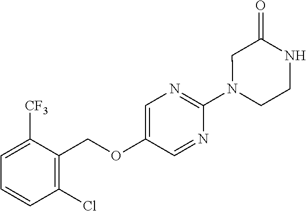 [SMILES: O=C1CN(c2ncc(OCc3c(Cl)cccc3C(F)(F)F)cn2)CCN1] | 1H NMR (399 MHz, DMSO) δ 8.30 (s, 2H), 8.00 (s, 1H), 7.86 (dd, J = 8.2, 1.2 Hz, 1H), 7.77 (dd, J = 7.9, 1.2 Hz, 1H), 7.62 (td, J = 8.0, 1.0 Hz, 1H), 5.16 (d, J = 1.3 Hz, 2H), 4.04 (s, 2H), 3.77 (dd, J = 6.4, 4.4 Hz, 2H), 3.21 (td, J = 5.4, 5.0, 2.6 Hz, 2H). | 387 |
| 303 | 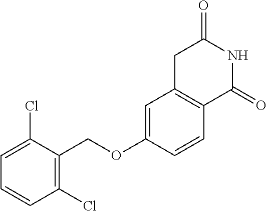 [SMILES: O=C1Cc2cc(OCc3c(Cl)cccc3Cl)ccc2C(=O)N1] | 1H NMR (400 MHz, DMSO) δ 7.81 (d, J = 8.0 Hz, 1H), 7.53 (d, J = 8.0 Hz, 2H), 7.43-7.39 (m, 1H), 6.81- 6.78 (m, 2H), 6.68 (dd, J = 8.8, 2.4 Hz, 1H), 4.46 (d, J = 4.4 Hz, 2H), 4.21 (s, 2H). | 335 |
| 304 | 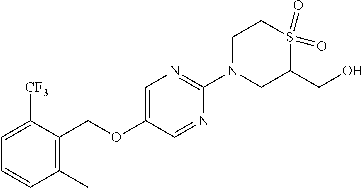 [SMILES: Cc1cccc(C(F)(F)F)c1COc1cnc(N2CCS(=O)(=O)C(CO)C2)nc1] | 1H NMR (400 MHz, DMSO) δ 8.39 (s, 2H), 7.64 (t, J = 6.8 Hz, 2H), 7.53 (t, J = 7.8 Hz, 1H), 5.16 (s, 2H), 5.12 (t, J = 5.8 Hz, 1H), 4.88 (d, J = 14.0 Hz, 1H), 4.70 (d, J = 14.6 Hz, 1H), 4.02-3.86 (m, 1H), 3.70-3.59 (m, 1H), 3.54 (m, J = 11.4, 8.4, 6.4 Hz, 1H), 3.45 (dd, J = 14.4, 10.2 Hz, 1H), 3.17 (dd, J = 12.2, 7.8 Hz, 3H), 2.45 (s, 3H). | 432 |
| 305 | 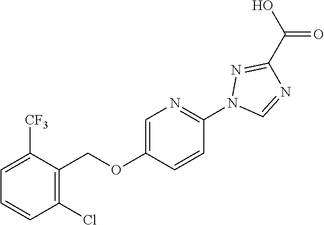 [SMILES: O=C(O)c1ncn(-c2ccc(OCc3c(Cl)cccc3C(F)(F)F)cn2)n1] | 1H NMR (400 MHz, DMSO) δ 9.07 (s, 1H), 8.35 (d, J = 2.1 Hz, 1H), 7.96 (d, J = 8.3 Hz, 1H), 7.86 (t, J = 8.8 Hz, 3H), 7.72 (t, J = 7.8 Hz, 1H), 7.28-7.13 (m, , 1H), 5.36 (s, 2H). | 399 |
| 306 | 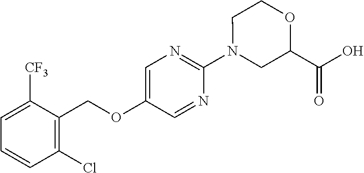 [SMILES: O=C(O)C1CN(c2ncc(OCc3c(Cl)cccc3C(F)(F)F)cn2)CCO1] | 1H NMR (400 MHz, DMSO-d6) δ 8.33 (s, 2H), 7.93 (d, J = 8.0 Hz, 1H), 7.84 (d, J = 7.9 Hz, 1H), 7.69 (t, J = 8.0 Hz, 1H), 5.21 (s, 2H), 4.41 (d, J = 13.2 Hz, 1H), 4.07 (d, J = 13.2 Hz, 1H), 3.93 (d, J = 11.4 Hz, 1H), 3.71 (d, J = 9.6 Hz, 1H), 3.56- 3.40 (m, 2H), 3.08-2.92 (m, 2H). | 418 |
| 307 | 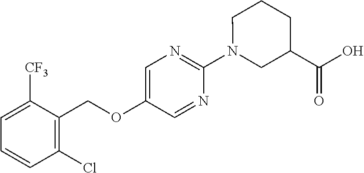 [SMILES: O=C(O)C1CCCN(c2ncc(OCc3c(Cl)cccc3C(F)(F)F)cn2)C1] | 1H NMR (400 MHz, DMSO) δ 12.25 (s, 1H), 8.31 (s, 2H), 7.93 (d, J = 8.1 Hz, 1H), 7.84 (d, J = 7.9 Hz, 1H), 7.69 (t, J = 8.0 Hz, 1H), 5.02 (s, 2H), 4.56 (dd, J = 12.5, 4.0 Hz, 1H), 4.34 (d, J = 13.1 Hz, 1H), 3.03 (s, 1H), 3.01- 2.92 (m, 1H), 2.37 (m, J = 10.7, 4.1 Hz, 1H), 2.01- 1.93 (m, 1H), 1.67 (m, J = | 416 |
| | | 17.7, 10.8, 3.6 Hz, 1H), 1.43 | |
| | | (t, J = 11.8 Hz, 1H), 1.24 (s, 1H). | |
| 308 | 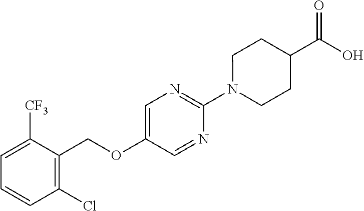 [SMILES: O=C(O)C1CCN(c2ncc(OCc3c(Cl)cccc3C(F)(F)F)cn2)CC1] | 1H NMR (400 MHz, DMSO-d6) δ 8.30 (s, 2H), 7.92 (d, J = 8.1 Hz, 1H), 7.84 (d, J = 7.8 Hz, 1H), 7.69 (t, J = 8.0 Hz, 1H), 5.20 (s, 2H), 4.48-4.33 (m, 2H), 3.36- 3.33 (m, 1H), 3.10-2.94 (m, 2H), 1.94-1.78 (m, 2H), 1.57-1.43 (m, 2H). | 416 |
| 309 | 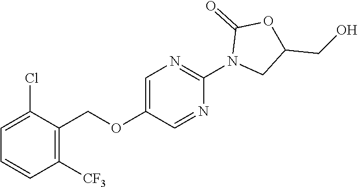 [SMILES: O=C1OC(CO)CN1c1ncc(OCc2c(Cl)cccc2C(F)(F)F)cn1] | 1H NMR (399 MHz, DMSO) δ 8.65 (s, 2H), 7.96 (dd, J = 8.2, 1.2 Hz, 1H), 7.87 (dd, J = 7.9, 1.2 Hz, 1H), 7.72 (td, J = 8.0, 0.9 Hz, 1H), 5.39-5.34 (m, 2H), 5.20 (t, J = 5.7 Hz, 1H), 4.66 (ddd, J = 9.7, 6.1, 3.1 Hz, 1H), 4.18 (dd, J = 9.9, 9.0 Hz, 1H), 3.97 (dd, J = 9.9, 6.3 Hz, 1H), 3.68 (ddd, J = 12.3, 5.6, 3.4 Hz, 1H), 3.57 (ddd, J = 12.3, 5.8, 4.1 Hz, 1H). | 404 |
| 310 | 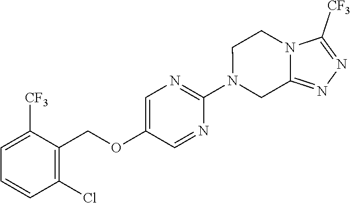 [SMILES: FC(F)(F)c1cccc(Cl)c1COc1cnc(N2CCn3c(nnc3C(F)(F)F)C2)nc1] | 1H NMR (399 MHz, DMSO) δ 8.45 (s, 2H), 7.94 (dd, J = 8.1, 1.2 Hz, 1H), 7.85 (dd, J = 8.0, 1.2 Hz, 1H), 7.70 (td, J = 8.0, 1.0 Hz, 1H), 5.26 (d, J = 1.2 Hz, 2H), 5.10 (s, 2H), 4.24 (tq, J = 7.2, 3.8, 3.1 Hz, 4H), 3.30 (s, 1H), 1.24 (s, 1H). | 479 |
| 311 | 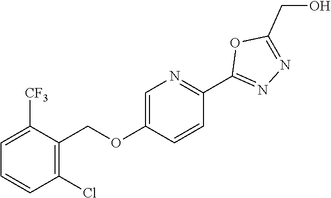 [SMILES: OCc1nnc(-c2ccc(OCc3c(Cl)cccc3C(F)(F)F)cn2)o1] | 1H NMR (399 MHz, DMSO) δ 8.58 (dd, J = 2.9, 0.6 Hz, 1H), 8.23-8.16 (m, 1H), 7.97 (dd, J = 8.1, 1.2 Hz, 1H), 7.92-7.81 (m, 2H), 7.74 (td, J = 8.0, 0.9 Hz, 1H), 5.99 (t, J = 6.3 Hz, 1H), 5.41 (d, J = 1.2 Hz, 2H), 4.74 (d, J = 4.1 Hz). | 386 |
| 312 | 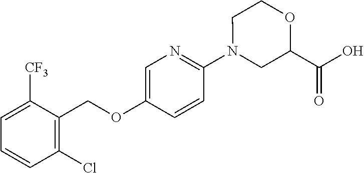 [SMILES: O=C(O)C1CN(c2ccc(OCc3c(Cl)cccc3C(F)(F)F)cn2)CCO1] | 1H NMR (400 MHz, DMSO) δ 7.99 (d, J = 2.8 Hz, 1H), 7.92 (d, J = 8.0 Hz, 1H), 7.83 (d, J = 7.6 Hz, 1H), 7.68 (t, J = 8.0 Hz, 1H), 7.40 (dd, J = 9.2, 3.2 Hz, 1H), 6.80 (d, J = 8.4 Hz, 1H), 5.16 (s, 2H), 4.11-4.05 (m, 2H), 3.79- 3.77 (m, 1H), 3.68-3.66 (m, 1H), 3.51-3.47 (m, 1H), 2.80- 2.75 (m, 1H), 2.67-2.66 (m, 1H). | 417 |
| 313 | 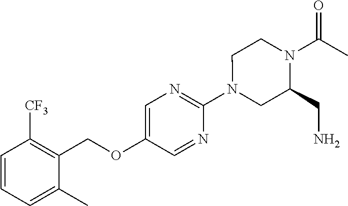 [SMILES: CC(=O)N1CCN(c2ncc(OCc3c(C)cccc3C(F)(F)F)cn2)C[C@@H]1CN] | 1H NMR (400 MHz, CDCl3) δ 8.18 (s, 2H), 7.58 (d, J = 6.8 Hz, 1H), 7.47- 7.46 (m, 1H), 7.42-7.38 (m, 1H), 5.09 (s, 2H), 4.60-4.51 (m, 2H), 3.53 (s, 2H), 3.33- 3.31 (m, 2H), 3.17 (s, 1H), 3.13-3.06 (m, 1H), 3.00- 2.95 (m, 1H), 2.50 (s, 3H), 2.06 (s, 3H). | 424 |
| 314 | 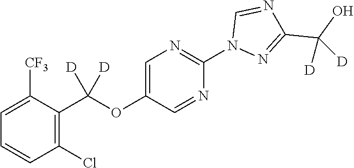 [SMILES: [2H]C([2H])(O)c1ncn(-c2ncc(OC([2H])([2H])c3c(Cl)cccc3C(F)(F)F)cn2)n1] | 1H NMR (400 MHz, DMSO-d6) δ 9.10 (s, 1H), 7.94 (d, J = 8.2 Hz, 1H), 7.86 (d, J = 7.9 Hz, 1H), 7.80- 7.75 (m, 2H), 7.71 (t, J = 8.0 Hz, 1H), 7.27-7.22 (m, 2H). | 390 |
| 315 | 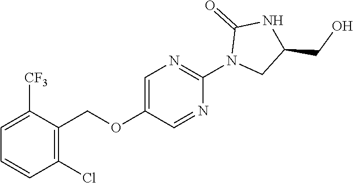 [SMILES: O=C1N[C@@H](CO)CN1c1ncc(OCc2c(Cl)cccc2C(F)(F)F)cn1] | 1H NMR (399 MHz, DMSO) δ 8.53 (s, 2H), 7.94 (dd, J = 8.2, 1.2 Hz, 1H), 7.85 (dd, J = 8.0, 1.2 Hz, 1H), 7.75-7.66 (m, 1H), 7.19 (s, 1H), 5.32 (s, 2H), 4.94 (t, J = 5.5 Hz, 1H), 4.00 (dd, J = 10.5, 9.0 Hz, 1H), 3.76 (dd, J = 10.5, 5.0 Hz, 1H), 3.65 (dt, J = 9.0, 5.1 Hz, 1H), 3.40 (q, J = 5.3 Hz, 2H). | 403 |
| 316 | 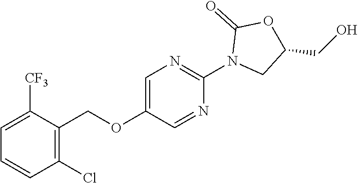 [SMILES: O=C1O[C@H](CO)CN1c1ncc(OCc2c(Cl)cccc2C(F)(F)F)cn1] | 1H NMR (399 MHz, DMSO) δ 8.65 (s, 2H), 7.99- 7.92 (m, 1H), 7.87 (dd, J = 8.0, 1.2 Hz, 1H), 7.72 (td, J = 8.0, 0.9 Hz, 1H), 5.37 (d, J = 1.2 Hz, 2H), 5.20 (t, J = 5.7 Hz, 1H), 4.66 (ddt, J = 9.7, 6.3, 3.1 Hz, 1H), 4.18 (dd, J = 9.9, 8.9 Hz, 1H), 3.97 (dd, J = 9.9, 6.3 Hz, 1H), 3.68 (ddd) | 404 |
| 317 | 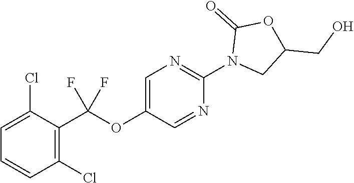 [SMILES: O=C1OC(CO)CN1c1ncc(OC(F)(F)c2c(Cl)cccc2Cl)cn1] | 1H NMR (400 MHz, DMSO) δ 8.81 (s, 2H), 7.71- 7.69 (m, 2H), 7.65-7.61 (m, 1H), 5.23 (s, 1H), 4.71- 4.65 (m, 1H), 4.21 (t, J = 10.0 Hz, 1H), 3.99 (dd, J = 10.0, 6.0 Hz, 1H), 3.68 (dd, J = 12.3, 3.0 Hz, 1H), 3.57 (dd, J = 12.2, 3.8 Hz, 1H). | 406 |
| 318 | 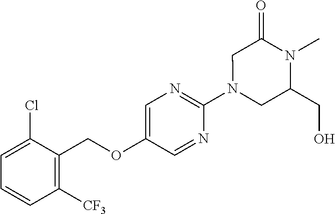 [SMILES: CN1C(=O)CN(c2ncc(OCc3c(Cl)cccc3C(F)(F)F)cn2)CC1CO] | 1H NMR (400 MHz, DMSO) δ 8.30 (s, 2H), 7.86 (dd, J = 8.2, 1.2 Hz, 1H) 7.77 (dd, J = 8.0, 1.2 Hz, 1H), 7.62 (td, J = 8.0, 0.9 Hz, 1H), 5.15 (d, J = 1.2 Hz, 2H), 4.87 (s, 1H), 4.48-4.39 (m, 1H), 4.25-4.21 (d, 1H), 3.84- 3.79 (d, 1H), 3.51-3.44 (m, 1H), 3.42-3.33 (m, 2H), 2.88 (s, 3H). | 431 |
| 319 | 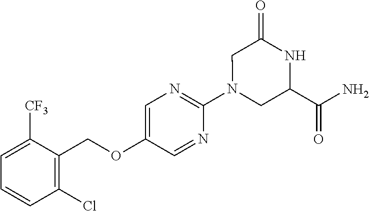 [SMILES: NC(=O)C1CN(c2ncc(OCc3c(Cl)cccc3C(F)(F)F)cn2)CC(=O)N1] | 1H NMR (400 MHz, DMSO-d6) δ 8.36 (s, 2H), 8.11 (d, J = 3.6 Hz, 1H), 7.93 (d, J = 8.0 Hz, 1H), 7.84 (d, J = 7.6 Hz, 1H), 7.69 (t, J = 8.0 Hz, 1H), 7.55 (s, 1H), 7.14 (s, 1H), 5.22 (s, 2H), 4.41 (dd, J = 13.2, 4.0 Hz, 1H), 4.22 (d, J = 18.0 Hz, 1H), 3.97-3.94 (m, 1H), 3.90 (d, J = 18.0 Hz, 2H), 3.71 (dd, J = 13.2, 4.0 Hz, 1H). | 430 |
| 320 | 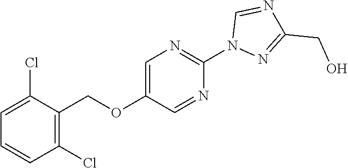 [SMILES: OCc1ncn(-c2ncc(OCc3c(Cl)cccc3Cl)cn2)n1] | 1H NMR (399 MHz, DMSO) δ 9.27 (s, 1H), 8.84 (s, 2H), 7.62 (d, J = 1.3 Hz, 1H), 7.60 (s, 1H), 7.52 (dd, J = 9.0, 7.1 Hz, 1H), 5.51 (s, 2H), 5.44 (t, J = 6.1 Hz, 1H), 4.55 (d, J = 6.1 Hz, 2H). | 352 |
| 321 | 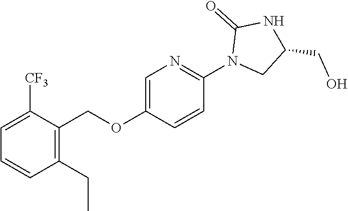 [SMILES: CCc1cccc(C(F)(F)F)c1COc1ccc(N2C[C@@H](CO)NC2=O)nc1] | 1H NMR (399 MHz, DMSO) δ 8.17-8.08 (m, 2H), 7.66 (d, J = 7.9 Hz, 2H), 7.63-7.55 (m, 1H), 7.52 (dd, J = 9.2, 3.1 Hz, 1H), 7.20 (s, 1H), 5.15 (s, 2H), 4.94 (t, J = 5.5 Hz, 1H), 3.99 (dd, J = 10.6, 9.2 Hz, 1H), 3.78 (dd, J = 10.6, 5.1 Hz, 1H), 3.69 (dq, J = 10.1, 5.2 Hz, 1H), 3.41 (h, J = 5.5 Hz, 2H), 2.77 (q, J = 7.5 Hz, 2H), 1.21 (t, J = 7.5 Hz, 3H). | 396 |
| 322 | 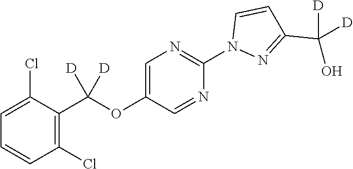 [SMILES: [2H]C([2H])(O)c1ccn(-c2ncc(OC([2H])([2H])c3c(Cl)cccc3Cl)cn2)n1] | 1H NMR (400 MHz, Chloroform-d) δ 8.90-8.38 (m, 3H), 7.50-7.35 (m, 2H), 7.30 (dd, J = 8.9, 7.2 Hz, 2H), 6.52 (d, J = 2.7 Hz, 1H). | 355 |
| 323 | 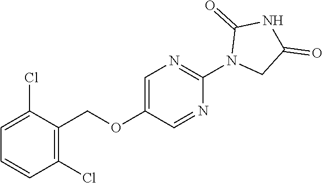 [SMILES: O=C1CN(c2ncc(OCc3c(Cl)cccc3Cl)cn2)C(=O)N1] | 1H NMR (399 MHz, DMSO) δ 11.22 (s, 1H), 8.63 (s, 2H), 7.60 (d, J = 1.2 Hz, 1H), 7.58 (s, 1H), 7.50 (dd, J = 9.0, 7.1 Hz, 1H), 5.40 (s, 2H), 4.47 (s, 2H). | 353 |
| 324 | 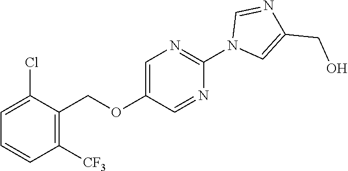 [SMILES: OCc1cn(-c2ncc(OCc3c(Cl)cccc3C(F)(F)F)cn2)cn1] | 1H NMR (399 MHz, DMSO) δ 8.88 (s, 2H), 8.52 (d, J = 1.5 Hz, 1H), 8.09- 8.03 (m, 1H), 8.03-7.94 (m, 1H), 7.87-7.76 (m, 2H), 5.52 (s, 2H), 5.14 (s, 1H), 4.52 (s, 2H). | 385 |
| 325 | 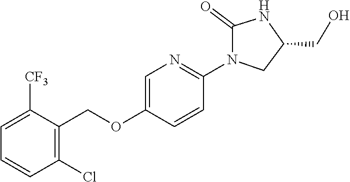 [SMILES: O=C1N[C@H](CO)CN1c1ccc(OCc2c(Cl)cccc2C(F)(F)F)cn1] | 1H NMR (399 MHz, DMSO) δ 8.17-8.09 (m, 2H), 7.93 (d, J = 8.0 Hz, 1H), 7.85 (d, J = 7.9 Hz, 1H), 7.70 (t, J = 8.0 Hz, 1H), 7.53 (dd, J = 9.2, 3.1 Hz, 1H), 7.21 (s, 1H), 5.23 (s, 2H), 4.94 (t, J = 5.5 Hz, 1H), 3.99 (dd, J = 10.6, 9.1 Hz, 1H), 3.77 (dd, J = 10.7, 5.1 Hz, 1H), 3.69 (dt, J = 9.6, 5.1 Hz, 1H), 3.41 (hept, J = 5.3 Hz, 2H), 0.99 | 402 |
| | | (d, J = 6.3 Hz, 5H), 0.97- | |
| | | 0.85 (m, 1H). | |
| 326 | 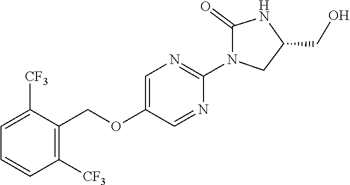 [SMILES: O=C1N[C@H](CO)CN1c1ncc(OCc2c(C(F)(F)F)cccc2C(F)(F)F)cn1] | 1H NMR (399 MHz, DMSO) δ 8.51 (s, 2H), 8.22 (d, J = 8.0 Hz, 2H), 7.93 (t, J = 8.0 Hz, 1H), 7.19 (s, 1H), 5.32 (s, 2H), 4.94 (t, J = 5.5 Hz, 1H), 4.00 (dd, J = 10.4, 9.0 Hz, 1H), 3.76 (dd, J = 10.5, 5.0 Hz, 1H), 3.65 (dq, J = 10.4, 5.4 Hz, 1H), 3.40 (q, J = 5.4 Hz, 2H). | 437 |
| 327 | 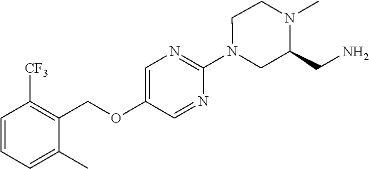 [SMILES: Cc1cccc(C(F)(F)F)c1COc1cnc(N2CCN(C)[C@@H](CN)C2)nc1] | 1H NMR (400 MHz, DMSO-d6) δ 8.30 (s, 2H), 7.75-7.59 (m, 2H), 7.53 (t, J = 7.7 Hz, 1H), 5.13 (s, 2H), 4.48-4.29 (m, 1H), 4.29- 4.14 (m, 1H), 3.02 (m, J = 13.1, 11.1, 3.1 Hz, 1H), 2.92 (dd, J = 13.0, 10.0 Hz, 1H), 2.85-2.75 (m, 2H), 2.73 (dd, J = 13.2, 6.3 Hz, 1H), 2.45 (s, 3H), 2.24 (s, 3H), | 396 |
| | | 2.21-2.11 (m, 1H), 2.01-2.00 | |
| | | (m, 1H). | |
| 328 | 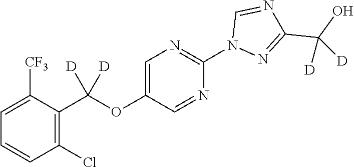 [SMILES: [2H]C([2H])(O)c1ncn(-c2ncc(OC([2H])([2H])c3c(Cl)cccc3C(F)(F)F)cn2)n1] | 1H NMR (400 MHz, CDCl3) δ 9.15 (s, 1H), 8.54 (s, 2H), 7.72 (dd, J = 8.0, 2.9 Hz, 2H), 7.53 (t, J = 8.0 Hz, 1H). | 390 |
| 329 | 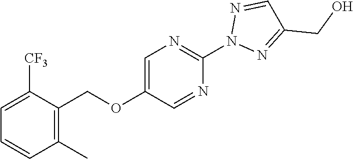 [SMILES: Cc1cccc(C(F)(F)F)c1COc1cnc(-n2ncc(CO)n2)nc1] | 1H NMR (399 MHz, DMSO) δ 8.84 (s, 2H), 8.07 (s, 1H), 7.72-7.64 (m, 2H), 7.58 (t, J = 7.8 Hz, 1H), 5.48 (t, J = 5.8 Hz, 1H), 5.41 (d, J = 1.3 Hz, 2H), 4.66 (d, J = 5.8 Hz, 2H), 2.47 (s, 3H). | 366 |
| 330 | 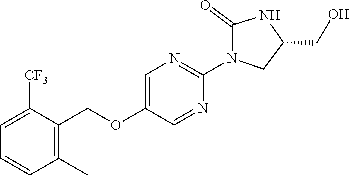 [SMILES: Cc1cccc(C(F)(F)F)c1COc1cnc(N2C[C@@H](CO)NC2=O)nc1] | 1H NMR (399 MHz, DMSO) δ 8.53 (s, 2H), 7.65 (dd, J = 7.8, 6.3 Hz, 2H), 7.59-7.51 (m, 1H), 7.18 (s, 1H), 5.27-5.22 (m, 2H), 4.94 (t, J = 5.5 Hz, 1H), 4.00 (dd, J = 10.4, 8.9 Hz, 1H), 3.77 (dd, J = 10.5, 5.0 Hz, 1H), 3.71-3.60 (m, 1H), 3.40 (hept, J = 5.7 Hz, 2H), 2.45 (s, 3H). | 383 |
| 331 | 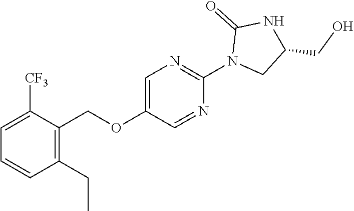 [SMILES: CCc1cccc(C(F)(F)F)c1COc1cnc(N2C[C@@H](CO)NC2=O)nc1] | 1H NMR (399 MHz, DMSO) δ 8.53 (s, 2H), 7.71- 7.64 (m, 2H), 7.64-7.56 (m, 1H), 7.18 (s, 1H), 5.24 (s, 2H), 4.95 (t, J = 5.5 Hz, 1H), 4.01 (dd, J = 10.5, 8.9 Hz, 1H), 3.77 (dd, J = 10.5, 5.0 Hz, 1H), 3.71-3.60 (m, 1H), 3.40 (hept, J = 5.7 Hz, 2H), 2.78 (q, J = 7.5 Hz, 2H), 1.22 (t, J = 7.5 Hz, 3H). | 397 |
| 332 | 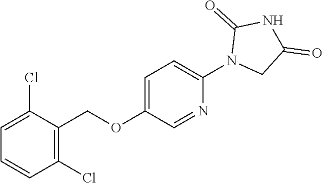 [SMILES: O=C1CN(c2ccc(OCc3c(Cl)cccc3Cl)cn2)C(=O)N1] | 1H NMR (399 MHz, DMSO) δ 11.25 (s, 1H), 8.19 (dd, J = 3.1, 0.7 Hz, 1H), 8.10 (dd, J = 9.1, 0.6 Hz, 1H), 7.65 (dd, J = 9.2, 3.0 Hz, 1H), 7.59 (d, J = 1.2 Hz, 1H), 7.57 (s, 1H), 7.49 (dd, J = 9.0, 7.1 Hz, 1H), 5.30 (s, 2H), 4.44 (s, 2H). | 352 |
| 333 | 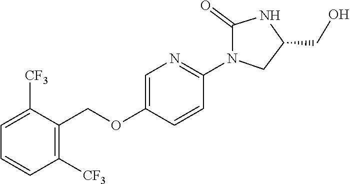 [SMILES: O=C1N[C@H](CO)CN1c1ccc(OCc2c(C(F)(F)F)cccc2C(F)(F)F)cn1] | 1H NMR (399 MHz, DMSO) δ 8.21 (d, J = 8.0 Hz, 2H), 8.13 (d, J = 9.2 Hz, 1H), 8.09 (d, J = 3.0 Hz, 1H), 7.92 (t, J = 8.0 Hz, 1H), 7.50 (dd, J = 9.2, 3.1 Hz, 1H), 7.21 (s, 1H), 5.24 (s, 2H), 4.94 (t, J = 5.5 Hz, 1H), 3.99 (dd, J = 10.6, 9.2 Hz, 1H), 3.77 (dd, J = 10.6, 5.1 Hz, 1H), 3.74- 3.63 (m, 1H), 3.48-3.34 (m, 2H). | 435 |
| 334 | 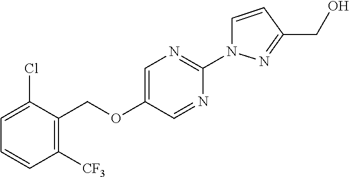 [SMILES: OCc1ccn(-c2ncc(OCc3c(Cl)cccc3C(F)(F)F)cn2)n1] | 1H NMR (399 MHz, DMSO) δ 8.76 (s, 2H), 8.50 (d, J = 2.6 Hz, 1H), 7.97 (dd, J = 8.1, 1.2 Hz, 1H), 7.88 (dd, J = 8.0, 1.2 Hz, 1H), 7.74 (td, J = 8.0, 0.9 Hz, 1H), 6.53 (d, J = 2.6 Hz, 1H), 5.44 (d, J = 1.3 Hz, 2H), 5.24 (t, J = 5.9 Hz, 1H), 4.53 (d, J = 5.9 Hz, 2H). | 385 |
| 335 | 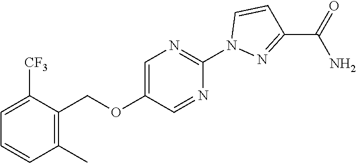 [SMILES: Cc1cccc(C(F)(F)F)c1COc1cnc(-n2ccc(C(N)=O)n2)nc1] | 1H NMR (399 MHz, DMSO) δ 8.82 (s, 2H), 8.63 (d, J = 2.6 Hz, 1H), 7.74 (s, 1H), 7.72-7.64 (m, 2H), 7.58 (t, J = 7.7 Hz, 1H), 7.46 (s, 1H), 6.91 (d, J = 2.6 Hz, 1H), 5.39 (s, 2H), 2.47 (s, 3H). | 378 |
| 336 | 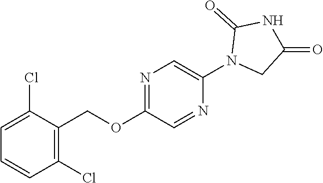 [SMILES: O=C1CN(c2cnc(OCc3c(Cl)cccc3Cl)cn2)C(=O)N1] | 1H NMR (399 MHz, DMSO) δ 11.41 (s, 1H), 8.96 (d, J = 1.5 Hz, 1H), 8.19 (d, J = 1.5 Hz, 1H), 7.61-7.54 (m, 2H), 7.48 (dd, J = 9.0, 7.1 Hz, 1H), 5.54 (s, 2H), 4.42 (s, 2H). | 355 |
| 337 | 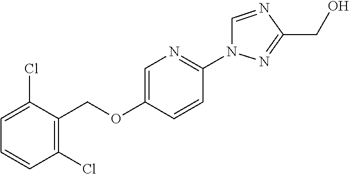 [SMILES: OCc1ncn(-c2ccc(OCc3c(Cl)cccc3Cl)cn2)n1] | 1H NMR (399 MHz, DMSO) δ 9.12 (s, 1H), 8.29 (dd, J = 2.8, 0.9 Hz, 1H), 7.82-7.71 (m, 2H), 7.53 (dd, J = 8.0, 0.9 Hz, 2H), 7.44 (dd, J = 8.9, 7.1 Hz, 1H), 5.34 (t, J = 6.1 Hz, 1H), 5.32 (s, 2H), 4.48 (d, J = 6.1 Hz, 2H). | 351 |
| 338 | 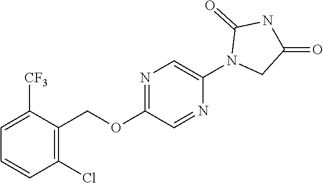 [SMILES: O=C1CN(c2cnc(OCc3c(Cl)cccc3C(F)(F)F)cn2)C(=O)N1] | 1H NMR (399 MHz, DMSO) δ 11.40 (s, 1H), 8.97 (d, J = 1.5 Hz, 1H), 8.19 (d, J = 1.5 Hz, 1H), 7.94 (dd, J = 8.1, 1.2 Hz, 1H), 7.86 (dd, J = 8.0, 1.2 Hz, 1H), 7.71 (td, J = 8.0, 0.9 Hz, 1H), 5.52 (d, J = 1.3 Hz, 2H), 4.44 (s, 2H). | 387 |
| 339 | 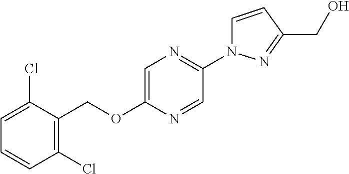 [SMILES: OCc1ccn(-c2cnc(OCc3c(Cl)cccc3Cl)cn2)n1] | 1H NMR (399 MHz, DMSO) δ 8.74 (d, J = 1.4 Hz, 1H), 8.43 (d, J = 2.5 Hz, 1H), 8.29 (d, J = 1.4 Hz, 1H), 7.61 (d, J = 1.2 Hz, 1H), 7.59 (s, 1H), 7.50 (dd, J = 8.9, 7.2 Hz, 1H), 6.56 (d, J = 2.5 Hz, 1H), 5.60 (s, 2H), 5.26 (t, J = 5.8 Hz, 1H), 4.55 (d, J = 5.8 Hz, 2H). | 353 |
| 340 | 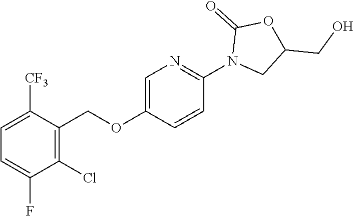 [SMILES: O=C1OC(CO)CN1c1ccc(OCc2c(C(F)(F)F)ccc(F)c2Cl)cn1] | 1H NMR (399 MHz, DMSO) δ 8.24-8.19 (m, 1H), 8.05 (d, J = 9.2 Hz, 1H), 7.71-7.55 (m, 3H), 5.32 (s, 2H), 5.20 (t, J = 5.6 Hz, 1H), 4.76-4.66 (m, 1H), 4.16 (dd, J = 10.0, 9.1 Hz, 1H), 3.96 (dd, J = 10.0, 6.3 Hz, 1H), 3.69 (ddd, J = 12.4, 5.5, 3.3 Hz, 1H), 5.37 (ddd, J = 12.4, 5.9, 4.1 Hz, 1H). | 387 |
| 341 | 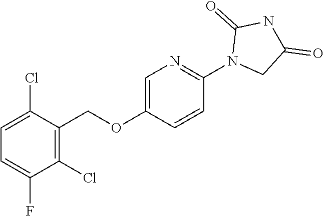 [SMILES: O=C1CN(c2ccc(OCc3c(Cl)ccc(F)c3Cl)cn2)C(=O)N1] | 1H NMR (399 MHz, DMSO) δ 11.26 (s, 1H), 8.22- 8.17 (m, 1H), 8.10 (d, J = 9.1 Hz, 1H), 7.72-7.55 (m, 3H), 5.32 (s, 2H), 4.44 (s, 2H). | 370 |
| 342 | 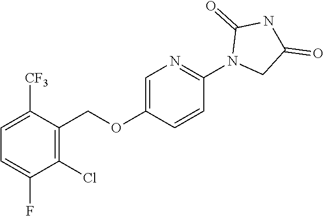 [SMILES: O=C1CN(c2ccc(OCc3c(C(F)(F)F)ccc(F)c3Cl)cn2)C(=O)N1] | 1H NMR (399 MHz, DMSO) δ 11.25 (s, 1H), 8.20 (dd, J = 3.1, 0.7 Hz, 1H), 8.11 (dd, J = 9.1, 0.6 Hz, 1H), 7.94 (dd, J = 8.9, 5.0 Hz, 1H), 7.77 (t, J = 8.6 Hz, 1H), 7.67 (dd, J = 9.2, 3.1 Hz, 1H), 5.30 (s, 2H), 4.45 (s, 2H). | 404 |
| 343 | 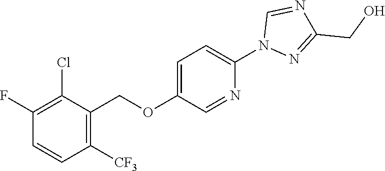 [SMILES: OCc1ncn(-c2ccc(OCc3c(C(F)(F)F)ccc(F)c3Cl)cn2)n1] | 1H NMR (399 MHz, DMSO) δ 9.19 (s, 1H), 8.37 (dd, J = 2.8, 0.7 Hz, 1H), 7.96 (dd, J = 8.9, 5.0 Hz, 1H), 7.92-7.75 (m, 3H), 5.42 (t, J = 6.1 Hz, 1H), 5.39 (s, 2H), 4.55 (d, J = 6.1 Hz, 2H). | 403 |
| 344 | 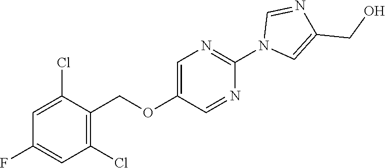 [SMILES: OCc1cn(-c2ncc(OCc3c(Cl)cc(F)cc3Cl)cn2)cn1] | 1H NMR (399 MHz, DMSO) δ 8.77 (s, 2H), 8.43 (d, J = 1.4 Hz, 1H), 7.74- 7.66 (m, 3H), 5.43 (s, 2H), 5.04 (t, J = 5.7 Hz, 1H), 4.43 (dd, J = 5.6, 1.0 Hz, 2H). | 369 |
| 345 | 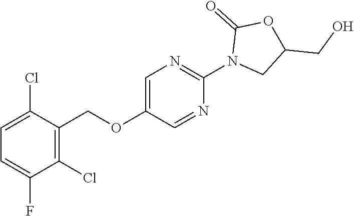 [SMILES: O=C1OC(CO)CN1c1ncc(OCc2c(Cl)ccc(F)c2Cl)cn1] | 1H NMR (399 MHz, DMSO) δ 8.64 (s, 2H), 7.74- 7.54 (m, 2H), 5.41 (d, J = 0.8 Hz, 2H), 5.19 (t, J = 5.7 Hz, 1H), 4.65 (ddt, J = 9.7, 6.3, 3.7 Hz, 1H), 4.17 (dd, J = 9.9, 9.0 Hz, 1H), 3.96 (dd, J = 9.9, 6.3 Hz, 1H), 3.67 (ddd, J = 12.3, 5.6, 3.4 Hz, 1H), 3.56 (ddd, J = 12.3, 5.8, 4.1 Hz, 1H). | 388 |
| 346 | 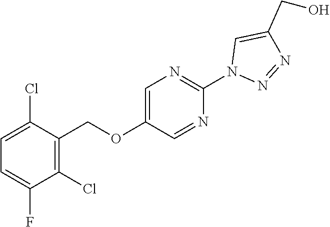 [SMILES: OCc1cn(-c2ncc(OCc3c(Cl)ccc(F)c3Cl)cn2)nn1] | 1H NMR (399 MHz, DMSO) δ 8.91 (s, 2H), 8.61 (s, 1H), 7.74-7.58 (m, 2H), 5.54 (s, 2H), 5.33 (t, J = 5.7 Hz, 1H), 4.62 (dd, J = 5.8, 0.7 Hz, 2H). | 370 |
| 347 | 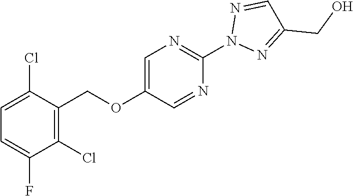 [SMILES: OCc1cnn(-c2ncc(OCc3c(Cl)ccc(F)c3Cl)cn2)n1] | 1H NMR (399 MHz, DMSO) δ 8.86 (s, 2H), 8.08 (s, 1H), 7.77-7.52 (m, 2H), 5.54 (s, 2H), 5.47 (s, 1H), 4.66 (d, J = 3.4 Hz, 2H). | 370 |
| 348 | 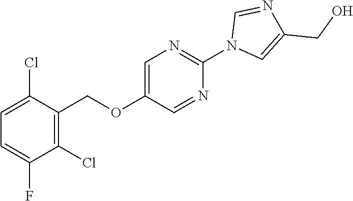 [SMILES: OCc1cn(-c2ncc(OCc3c(Cl)ccc(F)c3Cl)cn2)cn1] | 1H NMR (399 MHz, DMSO) δ 8.71 (s, 2H), 8.36 (d, J = 1.3 Hz, 1H), 7.64 (q, J = 1.1 Hz, 1H), 7.61 (dd, J = 9.0, 5.0 Hz, 1H), 7.56 (t, J = 8.8 Hz, 1H), 5.44-5.39 (m, 2H), 4.97 (t, J = 5.7 Hz, 1H), 4.36 (dd, J = 5.7, 1.0 Hz, 2H). | 368 |
| 349 | 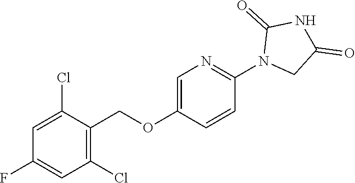 [SMILES: O=C1CN(c2ccc(OCc3c(Cl)cc(F)cc3Cl)cn2)C(=O)N1] | 1H NMR (399 MHz, DMSO) δ 11.26 (s, 1H), 8.20 (d, J = 3.0 Hz, 1H), 8.11 (d, J = 9.1 Hz, 1H), 7.71-7.61 (m, 3H), 5.28 (s, 2H), 4.46 (s, 2H). | 370 |
| 350 | 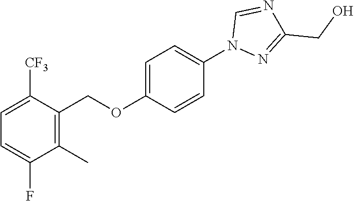 [SMILES: Cc1c(F)ccc(C(F)(F)F)c1COc1ccc(-n2cnc(CO)n2)cc1] | 1H NMR (399 MHz, DMSO) δ 9.14 (s, 1H), 7.80 (s, 1H), 7.81-7.71 (m, 2H), 7.49 (t, J = 8.9 Hz, 1H), 7.30- 7.21 (m, 2H), 5.74 (s, 6H), 5.21 (s). | 382 |
| 351 | 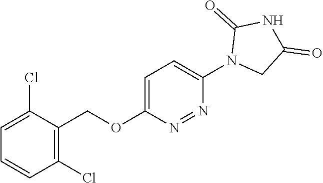 [SMILES: O=C1CN(c2ccc(OCc3c(Cl)cccc3Cl)nn2)C(=O)N1] | 1H NMR (399 MHz, DMSO) δ 11.46 (s, 1H), 8.40 (d, J = 9.6 Hz, 1H), 7.63- 7.55 (m, 2H), 7.49 (dd, J = 8.9, 7.1 Hz, 1H), 7.36 (d, J = 9.6 Hz, 1H), 5.65 (s, 2H), 4.54 (s, 2H). | 353 |
| 353 | 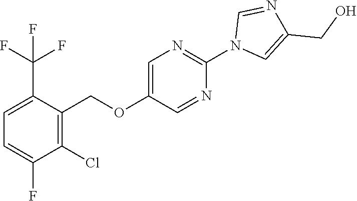 [SMILES: OCc1cn(-c2ncc(OCc3c(C(F)(F)F)ccc(F)c3Cl)cn2)cn1] | 1H NMR (399 MHz, DMSO) δ 8.84-8.78 (m, 1H), 8.80 (s, 2H), 8.43 (d, J = 1.4 Hz, 1H), 7.97 (dd, J = 8.9, 5.0 Hz, 1H), 7.80 (t, J = 8.7 Hz, 1H), 7.72 (q, J = 1.1 Hz, 1H), 5.47 (s, 2H), 5.05 (t, J = 5.7 Hz, 1H), 4.43 (dd, J = 5.7, 1.0 Hz, 2H). | 403 |
| 354 | 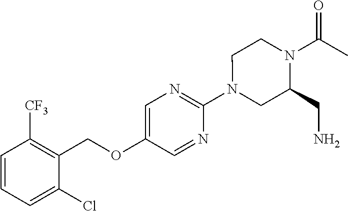 [SMILES: CC(=O)N1CCN(c2ncc(OCc3c(Cl)cccc3C(F)(F)F)cn2)C[C@@H]1CN] | N/A | 444 |
| 355 | 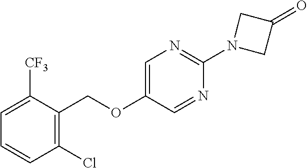 [SMILES: O=C1CN(c2ncc(OCc3c(Cl)cccc3C(F)(F)F)cn2)C1] | N/A | 358 |
| 358 | 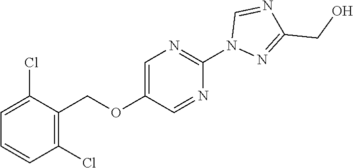 [SMILES: OCc1ncn(-c2ncc(OCc3c(Cl)cccc3Cl)cn2)n1] | 1H NMR (399 MHz, DMSO) δ 9.33 (s, 1H), 8.68 (s, 2H), 7.46 (d, J = 8.1 Hz, 2H), 7.30 (dd, J = 8.6, 7.5 Hz, 1H), 5.44 (t, J = 6.1 Hz, 1H), 4.54 (d, J = 6.0 Hz, 2H), 3.23 (dd, J = 8.3, 6.7 Hz, 2H), 2.98 (t, J = 7.5 Hz, 2H). | 350 |
| 359 | 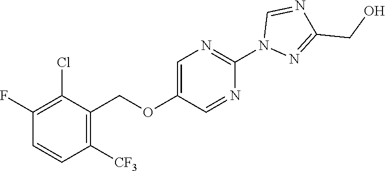 [SMILES: OCc1ncn(-c2ncc(OCc3c(C(F)(F)F)ccc(F)c3Cl)cn2)n1] | 1H NMR (399 MHz, DMSO) δ 9.27 (s, 1H), 8.86 (s, 2H), 7.98 (dd, J = 8.9, 5.0 Hz, 1H), 7.81 (t, J = 8.6 Hz, 1H), 5.51 (s, 2H), 5.44 (s, 1H), 4.55 (d, J = 3.3 Hz, 2H). | 404 |
| 360 | 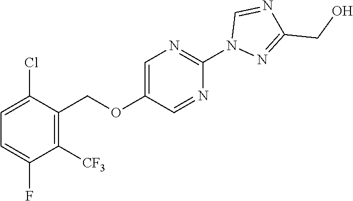 [SMILES: OCc1ncn(-c2ncc(OCc3c(Cl)ccc(F)c3C(F)(F)F)cn2)n1] | 1H NMR (399 MHz, DMSO) δ 9.27 (s, 1H), 8.84 (s, 2H), 8.05 (dd, J = 9.1, 4.7 Hz, 1H), 7.73 (ddd, J = 11.1, 9.1, 0.9 Hz, 1H), 5.52 (d, J = 1.5 Hz, 2H), 5.44 (t, J = 6.1 Hz, 1H), 4.55 (d, J = 6.1 Hz, 2H). | 404 |
| 361 | 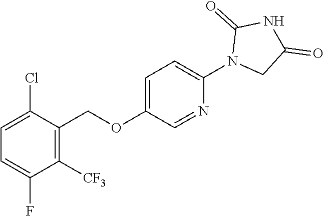 [SMILES: O=C1CN(c2ccc(OCc3c(Cl)ccc(F)c3C(F)(F)F)cn2)C(=O)N1] | 1H NMR (399 MHz, DMSO) δ 11.26 (s, 1H), 8.20-8.15 (m, 1H), 8.10 (d, J = 9.1 Hz, 1H), 8.01 (dd, J = 9.1, 4.7 Hz, 1H), 7.73- 7.64 (m, 1H), 7.64 (dd, J = 9.2, 3.1 Hz, 1H), 5.31 (d, J = 1.4 Hz, 2H), 4.44 (s, 2H). | 404 |
| 362 | 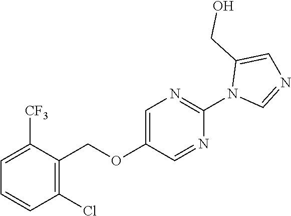 [SMILES: OCc1cncn1-c1ncc(OCc2c(Cl)cccc2C(F)(F)F)cn1] | 1H NMR (399 MHz, DMSO) δ 8.78 (s, 2H), 8.43 (d, J = 1.3 Hz, 1H), 7.72 (q, J = 1.0 Hz, 1H), 7.66-7.57 (m, 2H), 7.52 (dd, J = 9.0, 7.1 Hz, 1H), 5.47 (s, 2H), 5.04 (t, J = 5.7 Hz, 1H), 4.43 (dd, J = 5.8, 1.0 Hz, 2H). | 353 |
| 363 | 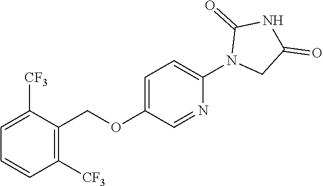 [SMILES: O=C1CN(c2ccc(OCc3c(C(F)(F)F)cccc3C(F)(F)F)cn2)C(=O)N1] | 1H NMR (399 MHz, DMSO) δ 11.31 (s, 1H), 8.22 (d, J = 8.0 Hz, 2H), 8.16 (d, J = 3.0 Hz, 1H), 8.12 (d, J = 9.1 Hz, 1H), 7.93 (t, J = 8.0 Hz, 1H), 7.62 (dd, J = 9.2, 3.1 Hz, 1H), 5.27 (s, 2H), 4.40 (s, 2H). | 420 |
| 366 | 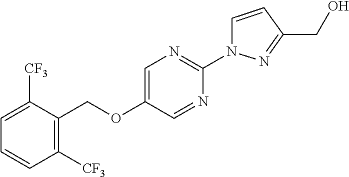 [SMILES: OCc1ccn(-c2ncc(OCc3c(C(F)(F)F)cccc3C(F)(F)F)cn2)n1] | 1H NMR (399 MHz, DMSO) δ 8.75 (s, 2H), 8.50 (d, J = 2.6 Hz, 1H), 8.25 (d, J = 8.0 Hz, 2H), 7.96 (t, J = 8.0 Hz, 1H), 6.53 (d, J = 2.6 Hz, 1H), 5.44 (s, 2H), 5.24 (t, J = 5.8 Hz, 1H), 4.53 (d, J = 5.0 Hz, 2H). | 419 |
| 367 | 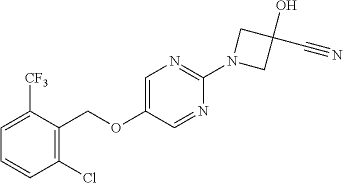 [SMILES: N#CC1(O)CN(c2ncc(OCc3c(Cl)cccc3C(F)(F)F)cn2)C1] | N/A | 385 |
| 368 | 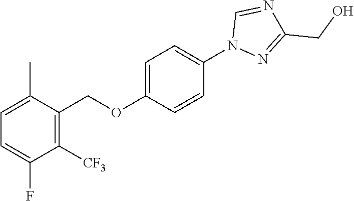 [SMILES: Cc1ccc(F)c(C(F)(F)F)c1COc1ccc(-n2cnc(CO)n2)cc1] | 1H NMR (399 MHz, DMSO) δ 9.11 (s, 1H), 7.83- 7.74 (m, 2H), 7.69 (dd, J = 8.7, 5.3 Hz, 1H), 7.48 (dd, J = 11.8, 8.6 Hz, 1H), 7.27- 7.19 (m, 2H), 5.36 (t, J = 6.1 Hz, 1H), 5.18 (d, J = 1.6 Hz, 2H), 4.52 (d, J = 6.1 Hz, 2H), 2.41 (s, 3H). | 382 |
| 369 | 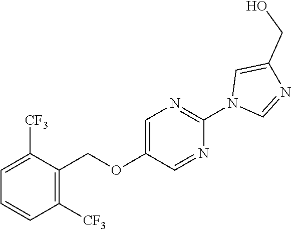 [SMILES: OCc1cn(-c2ncc(OCc3c(C(F)(F)F)cccc3C(F)(F)F)cn2)cn1] | 1H NMR (399 MHz, DMSO) δ 8.78 (s, 2H), 8.43 (d, J = 1.3 Hz, 1H), 8.25 (d, J = 8.0 Hz, 2H), 7.96 (t, J = 8.0 Hz, 1H), 7.71 (d, J = 1.3 Hz, 1H), 5.44 (s, 2H), 5.05 (t, J = 5.7 Hz, 1H), 4.43 (d, J = 5.5 Hz, 2H). | 419 |
| 370 | 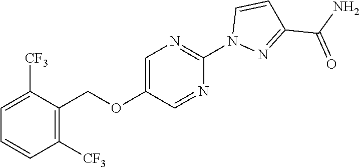 [SMILES: NC(=O)c1ccn(-c2ncc(OCc3c(C(F)(F)F)cccc3C(F)(F)F)cn2)n1] | 1H NMR (399 MHz, DMSO) δ 8.83 (s, 2H), 8.63 (d, J = 2.7 Hz, 1H), 8.25 (d, J = 8.1 Hz, 2H), 7.97 (t, J = 8.0 Hz, 1H), 7.75 (s, 1H), 7.46 (s, 1H), 6.91 (d, J = 2.7 Hz, 1H), 5.48 (s, 2H). | 432 |

### Example A—Measuring CFTR-PTC mutant Translation Readthrough using Nanoluc tagged CFTR Luminescence

16HBE human bronchial epithelial cells (stably over-expressing recombinant human CFTR carrying the premature stop codon (PTC) mutation G542X or R1162X along with a Nanoluc tag incorporated into the CFTR channel within extracellular loop 4 in G542X cell line or C-terminus end in the R1162X cell line were seeded at 0.01×10⁶ cells/well and grown in Minimal Essential Medium (MEM) as monolayers in 384 well plates and incubated at 37° C. with 5% CO₂. Media was changed every other day until day of experiment. Agents to be evaluated were made up in separate 384 well, polypropylene plates at a 2X the final desired test concentration. Test agent 10-point concentration response curves were constructed using the Labcyte Echo 555 acoustic dispenser. Test agents were typically tested at a max final test concentrations of 50 μM with sequential 2-fold lower concentrations below this in the absence or presence of a fixed 10 μM concentration of the aminoglycoside G418 made up in MEM growth media (reduced Serum Media+HEPES Buffer+2.4 g/L Sodium Bicarbonate+L-Glutamine containing CFTR corrector VX-809 (3 μM)). Treatment with 10 μM G418 alone produced approximately 10% of the response observed with a maximally effective concentration of G418 (typically 125-250 μM). Test agent effects in presence of 10 μM G418 were compared to control wells on the plate containing a maximally effective concentration of G418 (typically 125-250 μM) made up in the same media. 25 ul of test agents/control solutions were transferred from the compound plate to the cell plate, which already contained 25 ul/well of cell plating media, using an Integra Viaflo 384. Cell plates were incubated with the compounds for 48 hours, at which time they were processed using Nanoluc luminescence Kit (Promega Nano-Glo Live Cell Assay System) to measure CFTR-PTC mutant translation readthrough. Luminescence was measured as follows. Cell plates were removed from the incubator and allowed to equilibrate to room temperature for 20 minutes. Media was removed from all wells and the plates were blotted to remove excess remaining volume. Nano-Glo Live Cell Substrate was diluted 1:20 in the appropriate amount of Nano-Glo LCS Dilution Buffer to accommodate the number of cell plates being tested. The substrate/dilution buffer mix was then diluted 1:3 with MEM media to make the appropriate volume for the number of plates being tested. The substrate/dilution buffer/OptiMEM mix was added across the Nanoluc testing cell plates and the plates were read for luminescence signal immediately. Test agent effects on CFTR translation readthrough was determined by normalizing any observed increase in luminescence induced by 48 hours pretreatment with test compound to the magnitude of luminescence increase produced by pretreatment with a maximally effective concentration of the reference readthrough enhancing agent G418 (125-250 μM). The data for selected readthrough modulator compounds in combination with the aminoglycoside G418 is provided in Table 1 below.

TABLE 1
CFTR-PTC mutant Translation Readthrough of Selected Compounds in Combination
with G418

| | | CFTR | | CFTR |
|---|---|---|---|---|
| | CFTR | R1162X | CFTR | G542X |
| | R1162X | ACTIVITY | G542X | ACTIVITY |
| | ACTIVITY | Fold 10 μM | ACTIVITY | Fold 10 μM |
| Structure Name | EC50 (uM) | G-418 | EC50 (uM) | G-418 |
| 3-[4-[(2,6- | B | B′ | B | B′ |
| dichlorophenyl)methoxy]phenyl]-5- | | | | |
| (hydroxymethyl)oxazolidin-2-one | | | | |
| (5S)-3-[4-[(2,6- | B | B′ | B | B′ |
| dichlorophenyl)methoxy]phenyl]-5- | | | | |
| (hydroxymethyl)oxazolidin-2-one | | | | |
| (5R)-3-[4-[(2,6- | B | A′ | B | A′ |
| dichlorophenyl)methoxy]phenyl]-5- | | | | |
| (hydroxymethyl)oxazolidin-2-one | | | | |
| 3-[4-[(2,6- | C | A′ | B | B′ |
| dimethylphenyl)methoxy]phenyl]-5- | | | | |
| (hydroxymethyl)oxazolidin-2-one | | | | |
| 3-[4-[(3,5- | C | B′ | B | B′ |
| dichlorophenyl)methoxy]phenyl]-5- | | | | |
| (hydroxymethyl)oxazolidin-2-one | | | | |
| [1-[4-[(2,6- | B | A′ | A | A′ |
| dichlorophenyl)methoxy]phenyl] | | | | |
| imidazol-4-yl]methanol | | | | |
| 3-[5-[(2,6-dichlorophenyl)methoxy]- | B | A′ | B | A′ |
| 2-pyridyl]-5- | | | | |
| (hydroxymethyl)oxazolidin-2-one | | | | |
| 3-[4-[2-(2,6- | C | B′ | B | C′ |
| dichlorophenyl)ethyl]phenyl]-5- | | | | |
| (hydroxymethyl)oxazolidin-2-one | | | | |
| 3-[4-[(2,6-dichlorophenyl)methoxy]- | B | C′ | B | B′ |
| 3-fluoro-phenyl]-5- | | | | |
| (hydroxymethyl)oxazolidin-2-one | | | | |
| 3-[4-[(2,6- | B | B′ | B | A′ |
| dichlorophenyl)methoxy]phenyl] | | | | |
| oxazolidin-2-one | | | | |
| 1-[4-[(2,6- | B | B′ | B | B′ |
| dichlorophenyl)methoxy]phenyl]-4- | | | | |
| (methoxymethyl)imidazolidin-2-one | | | | |
| 5-(hydroxymethyl)-3-[4-[[2-methyl- | B | A′ | B | A′ |
| 6- | | | | |
| (trifluoromethyl)phenyl]methoxy] | | | | |
| phenyl]oxazolidin-2-one | | | | |
| 3-[4-[(2-chloro-6-methoxy- | C | C′ | C | C′ |
| phenyl)methoxy]phenyl]-5- | | | | |
| (hydroxymethyl)oxazolidin-2-one | | | | |
| 3-[4-[(2,6- | C | C′ | C | — |
| dimethylphenyl)methoxy]-1- | | | | |
| naphthyl]-5- | | | | |
| (hydroxymethyl)oxazolidin-2-one | | | | |
| [1-[5-[(2,6- | B | A′ | A | A′ |
| dichlorophenyl)methoxy]pyrimidin- | | | | |
| 2-yl]imidazol-4-yl]methanol | | | | |
| 3-[4-[(2-chloro-6-cyclopropyl- | B | A′ | B | A′ |
| phenyl)methoxy]phenyl]-5- | | | | |
| (hydroxymethyl)oxazolidin-2-one | | | | |
| 3-[4-[[2-chloro-6- | C | B′ | B | C′ |
| (difluoromethoxy)phenyl]methoxy] | | | | |
| phenyl]-5- | | | | |
| (hydroxymethyl)oxazolidin-2-one | | | | |
| 1-[4-[(2,6- | B | A′ | B | A′ |
| dichlorophenyl)methoxy]phenyl]- | | | | |
| 1,2,4-triazole | | | | |
| 4-[4-[(2,6- | B | A′ | B | A′ |
| dichlorophenyl)methoxy]phenyl]-1- | | | | |
| methyl-imidazole | | | | |
| 3-[4-[(2-bromo-6-methoxy- | C | B′ | C | A′ |
| phenyl)methoxy]phenyl]-5- | | | | |
| (hydroxymethyl)oxazolidin-2-one | | | | |
| 3-[5-[(2,6- | C | B′ | B | B′ |
| dichlorophenyl)methoxy]pyrazin-2- | | | | |
| yl]-5-(hydroxymethyl)oxazolidin-2- | | | | |
| one | | | | |
| [1-[4-[(2,6- | B | A′ | B | A′ |
| dichlorophenyl)methoxy]phenyl] | | | | |
| pyrazol-4-yl]methanol | | | | |
| 3-[4-[(2,6- | B | A′ | B | A′ |
| dibromophenyl)methoxy]phenyl]-5- | | | | |
| (hydroxymethyl)oxazolidin-2-one | | | | |
| [3-[4-[(2,6- | B | A′ | B | A′ |
| dichlorophenyl)methoxy]phenyl] | | | | |
| isoxazol-5-yl]methanol | | | | |
| [3-[5-[(2,6- | B | A′ | A | A′ |
| dichlorophenyl)methoxy]-2- | | | | |
| pyridyl]isoxazol-5-yl]methanol | | | | |
| [1-[4-[(2,6- | B | A′ | A | A′ |
| dichlorophenyl)methoxy]phenyl] | | | | |
| pyrazol-3-yl]methanol | | | | |
| 1-[4-[(2,6- | C | A′ | B | B′ |
| dichlorophenyl)methoxy]phenyl] | | | | |
| imidazole | | | | |
| 1-[4-[(2,6- | B | B′ | B | A′ |
| dichlorophenyl)methoxy]phenyl] | | | | |
| pyrrolidine-2,5-dione | | | | |
| [4-[4-[(2,6- | B | A′ | B | A′ |
| dichlorophenyl)methoxy]phenyl] | | | | |
| oxazol-2-yl]methanol | | | | |
| [1-[4-[(2,6- | B | A′ | B | A′ |
| dichlorophenyl)methoxy]phenyl]- | | | | |
| 1,2,4-triazol-3-yl]methanol | | | | |
| 6-[(2,6-dichlorophenyl)methoxy]- | B | A′ | C | B′ |
| 2H-phthalazin-1-one | | | | |
| 3-[4-[(2,6- | B | C′ | B | B′ |
| dichlorophenyl)methoxy]phenyl] | | | | |
| oxazolidine-2,4-dione | | | | |
| [1-[5-[(2,6- | C | A′ | B | B′ |
| dichlorophenyl)methoxy]-2- | | | | |
| pyridyl]imidazol-4-yl]methanol | | | | |
| 2-[4-[4-[(2,6- | B | A′ | B | B′ |
| dichlorophenyl)methoxy]phenyl] | | | | |
| triazol-1-yl]ethanol | | | | |
| 5-[(2,6-dichlorophenyl)methoxy]-2- | B | A′ | B | A′ |
| (1-methyltriazol-4-yl)pyridine | | | | |
| [4-[4-[(2,6- | B | A′ | B | A′ |
| dichlorophenyl)methoxy]phenyl] | | | | |
| morpholin-2-yl]methanol | | | | |
| [1-[5-[(2,6- | A | A′ | A | A′ |
| dichlorophenyl)methoxy]-2- | | | | |
| pyridyl]triazol-4-yl]methanol | | | | |
| 3-[5-[(3,5-dichlorophenyl)methoxy]- | B | A′ | B | A′ |
| 2-pyridyl]-5- | | | | |
| (hydroxymethyl)oxazolidin-2-one | | | | |
| [1-[4-[(3,5- | C | C′ | B | — |
| dichlorophenyl)methoxy]phenyl] | | | | |
| imidazol-4-yl]methanol | | | | |
| 4-[4-[(2,6- | B | B′ | B | B′ |
| dichlorophenyl)methoxy]phenyl]-3- | | | | |
| (hydroxymethyl)-2H-furan-5-one | | | | |
| N-[[3-[4-[[2-methyl-6- | B | A′ | B | A′ |
| (trifluoromethyl)phenyl]methoxy] | | | | |
| phenyl]-2-oxo-oxazolidin-5- | | | | |
| yl]methyl]acetamide | | | | |
| [1-[5-[[2-methyl-6- | B | A′ | A | A′ |
| (trifluoromethyl)phenyl]methoxy]-2- | | | | |
| pyridyl]imidazol-4-yl]methanol | | | | |
| [1-[[1-[5-[[2-methyl-6- | B | A′ | B | A′ |
| (trifluoromethyl)phenyl]methoxy]-2- | | | | |
| pyridyl]imidazol-4- | | | | |
| yl]methyl]imidazol-4-yl]methanol | | | | |
| [1-[5-[[2-methyl-6- | A | A′ | A | A′ |
| (trifluoromethyl)phenyl]methoxy] | | | | |
| pyrimidin-2-yl]imidazol-4-yl]methanol | | | | |
| [1-[5-[(2,6- | B | A′ | B | A′ |
| dimethylphenyl)methoxy]-2- | | | | |
| pyridyl]pyrazol-3-yl]methanol | | | | |
| 5-[5-[(2,6- | B | A′ | B | A′ |
| dichlorophenyl)methoxy]pyrimidin- | | | | |
| 2-yl]-2-oxa-5- | | | | |
| azabicyclo[2.2.1]heptane | | | | |
| [1-[5-[(2,6- | B | A′ | A | A′ |
| dichlorophenyl)methoxy]pyrimidin- | | | | |
| 2-yl]pyrazol-3-yl]methanol | | | | |
| 3-[4-[(2,6-dichloro-4-fluoro- | C | B′ | C | B′ |
| phenyl)methoxy]phenyl]-5- | | | | |
| (hydroxymethyl)oxazolidin-2-one | | | | |
| [1-[5-[2-methyl-6- | A | A′ | A | A′ |
| (trifluoromethyl)phenyl]methoxy] | | | | |
| pyrimidin-2-yl]pyrazol-3-yl]methanol | | | | |
| 2-[3-[[5-[(2,6- | B | A′ | B | A′ |
| dichlorophenyl)methoxy]-2- | | | | |
| pyridyl]amino]pyrazol-1-yl]ethanol | | | | |
| 3-[5-[[2-chloro-6- | B | A′ | A | A′ |
| (trifluoromethyl)phenyl]methoxy]-2- | | | | |
| pyridyl]-5- | | | | |
| (hydroxymethyl)oxazolidin-2-one | | | | |
| [4-[5-[(2,6- | B | A′ | A | A′ |
| dichlorophenyl)methoxy]pyrimidin- | | | | |
| 2-yl]morpholin-2-yl]methanol | | | | |
| [1-[5-[[2-chloro-6- | B | A′ | A | A′ |
| (trifluoromethyl)phenyl]methoxy]-2- | | | | |
| pyridyl]imidazol-4-yl]methanol | | | | |
| 5-(aminomethyl)-3-[4-[(2,6- | C | A′ | B | B′ |
| dichlorophenyl)methoxy]phenyl] | | | | |
| oxazolidin-2-one | | | | |
| 3-[4-[(2,6-dichloro-3-fluoro- | B | B′ | B | A′ |
| phenyl)methoxy]phenyl]-5- | | | | |
| (hydroxymethyl)oxazolidin-2-one | | | | |
| 2-[3-[[5-[(2,6- | B | A′ | B | A′ |
| dichlorophenyl)methoxy]-2- | | | | |
| pyridyl]amino]-1,2,4-triazol-1- | | | | |
| yl]ethanol | | | | |
| [5-[4-[(2,6- | A | A′ | A | A′ |
| dichlorophenyl)methoxy]phenyl]- | | | | |
| 1,3,4-oxadiazol-2-yl]methanol | | | | |
| 3-[5-[2-(2,6-dichlorophenyl)ethyl]- | C | A′ | B | B′ |
| 2-pyridyl]-5- | | | | |
| (hydroxymethyl)oxazolidin-2-one | | | | |
| [1-[5-[2-(2,6-dichlorophenyl)ethyl]- | C | ′ | B | B′ |
| 2-pyridyl]imidazol-4-yl]methanol | | | | |
| 1-[5-[(2,6- | B | A′ | B | A′ |
| dichlorophenyl)methoxy]pyrimidin- | | | | |
| 2-yl]-1-methyl-urea | | | | |
| [3-[5-[(2,6- | B | A′ | B | A′ |
| dichlorophenyl)methoxy]-2-pyridyl]- | | | | |
| 1,2,4-oxadiazol-5-yl]methanol | | | | |
| 3-[4-[(2,6- | C | B′ | B | A′ |
| dichlorophenyl)methoxy]phenyl]-5- | | | | |
| (1-hydroxyethyl)oxazolidin-2-one | | | | |
| [4-[5-[[2-chloro-6- | A | A′ | A | A′ |
| (trifluoromethyl)phenyl]methoxy]-2- | | | | |
| pyridyl]morpholin-2-yl]methanol | | | | |
| [1-[4-[2-(2,6- | C | A′ | B | B′ |
| dichlorophenyl)ethyl]phenyl] | | | | |
| imidazol-4-yl]methanol | | | | |
| [4-[4-[[2-methyl-6- | B | A′ | A | A′ |
| (trifluoromethyl)phenyl]methoxy] | | | | |
| phenyl]morpholin-2-yl]methanol | | | | |
| [4-[5-[(2,6- | B | A′ | B | A′ |
| dichlorophenyl)methoxy]-2- | | | | |
| pyridyl]morpholin-2-yl]methanol | | | | |
| [(2S)-4-[5-[(2,6- | A | A′ | A | A′ |
| dichlorophenyl)methoxy]pyrimidin- | | | | |
| 2-yl]morpholin-2-yl]methanol | | | | |
| [(2R)-4-[5-[(2,6- | B | A′ | B | A′ |
| dichlorophenyl)methoxy]pyrimidin- | | | | |
| 2-yl]morpholin-2-yl]methanol | | | | |
| [1-[4-[[2-methyl-6- | B | A′ | B | A′ |
| (trifluoromethyl)phenyl]methoxy] | | | | |
| phenyl]triazol-4-yl]methanol | | | | |
| [1-[4-[[2-chloro-6- | B | A′ | B | A′ |
| (trifluoromethyl)phenyl]methoxy] | | | | |
| phenyl]triazol-4-yl]methanol | | | | |
| 1-[5-[(2,6- | B | A′ | B | A′ |
| dichlorophenyl)methoxy]pyrimidin- | | | | |
| 2-yl]azetidin-3-ol | | | | |
| [5-[5-[(2,6- | B | B′ | B | B′ |
| dichlorophenyl)methoxy]-2-pyridyl]- | | | | |
| 1H-1,2,4-triazol-3-yl]methanol | | | | |
| [1-[4-[[2-methyl-6- | A | A′ | A | A′ |
| (trifluoromethyl)phenyl]methoxy] | | | | |
| phenyl]-1,2,4-triazol-3-yl]methanol | | | | |
| 5-[(2,6- | B | B′ | B | A′ |
| dichlorophenyl)methoxy]pyridine-2- | | | | |
| carboxamide | | | | |
| 1-[4-[5-[(2,6- | B | A′ | B | A′ |
| dichlorophenyl)methoxy]pyrimidin- | | | | |
| 2-yl]piperazin-1-yl]ethanone | | | | |
| 4-[5-[(2,6- | B | A′ | B | A′ |
| dichlorophenyl)methoxy]pyrimidin- | | | | |
| 2-yl]-1-methyl-piperazin-2-one | | | | |
| [4-[5-[[2-chloro-6- | A | A′ | A | A′ |
| (trifluoromethyl)phenyl]methoxy] | | | | |
| pyrimidin-2-yl]morpholin-2- | | | | |
| yl]methanol | | | | |
| [4-[5-[[2-methyl-6- | A | A′ | A | A′ |
| (trifluoromethyl)phenyl]methoxy] | | | | |
| pyrimidin-2-yl]morpholin-2- | | | | |
| yl]methanol | | | | |
| [4-[5-[[2-fluoro-6- | B | A′ | B | A′ |
| (trifluoromethyl)phenyl]methoxy] | | | | |
| pyrimidin-2-yl]morpholin-2- | | | | |
| yl]methanol | | | | |
| [4-[5-[(2,6- | B | A′ | B | A′ |
| dichlorophenyl)methoxy]pyrimidin- | | | | |
| 2-yl]oxazol-2-yl]methanol | | | | |
| [4-[5-[(2-chloro-6-cyclopropyl- | A | A′ | A | A′ |
| phenyl)methoxy]pyrimidin-2- | | | | |
| yl]morpholin-2-yl]methanol | | | | |
| [1-[5-[[2-chloro-6- | B | A′ | B | A′ |
| (trifluoromethyl)phenyl]methoxy] | | | | |
| pyrimidin-2-yl]triazol-4-yl]methanol | | | | |
| (3R,4S)-1-[5-[(2,6- | C | A′ | B | A′ |
| dichlorophenyl)methoxy]pyrimidin- | | | | |
| 2-yl]pyrrolidine-3,4-diol | | | | |
| [1-[5-[(2,6- | B | A′ | B | A′ |
| dichlorophenyl)methoxy]pyrimidin- | | | | |
| 2-yl]triazol-4-yl]methanol | | | | |
| [2-[5-[(2,6- | B | A′ | B | A′ |
| dichlorophenyl)methoxy]pyrimidin- | | | | |
| 2-yl]triazol-4-yl]methanol | | | | |
| [1-[5-[[2-methyl-6- | B | A′ | B | A′ |
| (trifluoromethyl)phenyl]methoxy] | | | | |
| pyrimidin-2-yl]triazol-4-yl]methanol | | | | |
| [4-[5-[[2-methyl-6- | B | A′ | B | A′ |
| (trifluoromethyl)phenyl]methoxy]-2- | | | | |
| pyridyl]morpholin-2-yl]methanol | | | | |
| 5-[[2-methyl-6- | B | A′ | B | A′ |
| (trifluoromethyl)phenyl]methoxy]-2- | | | | |
| piperazin-1-yl-pyrimidine | | | | |
| [1-[4-[[2-fluoro-3-methyl-6- | C | B′ | B | B′ |
| (trifluoromethyl)phenyl]methoxy] | | | | |
| phenyl]triazol-4-yl]methanol | | | | |
| [6-[[2-methyl-6- | C | A′ | C | A′ |
| (trifluoromethyl)phenyl]methoxy] | | | | |
| imidazo[1,2-a]pyridin-2-yl]methanol | | | | |
| 7-[5-[(2,6- | B | B′ | B | A′ |
| dichlorophenyl)methoxy]pyrimidin- | | | | |
| 2-yl]-5,6,8,8a-tetrahydro-1H- | | | | |
| oxazolo[3,4-a]pyrazin-3-one | | | | |
| 4-[5-[[2-methyl-6- | A | A′ | A | A′ |
| (trifluoromethyl)phenyl]methoxy] | | | | |
| pyrimidin-2-yl]piperazine-1- | | | | |
| carboxamide | | | | |
| [(2S)-4-[5-[[2-methyl-6- | A | A′ | A | A′ |
| (trifluoromethyl)phenyl]methoxy] | | | | |
| pyrimidin-2-yl]morpholin-2- | | | | |
| yl]methanol | | | | |
| [(2S)-4-[5-[[2-chloro-6- | A | A′ | A | A′ |
| (trifluoromethyl)phenyl]methoxy] | | | | |
| pyrimidin-2-yl]morpholin-2- | | | | |
| yl]methanol | | | | |
| [(2R)-4-[5-[[2-methyl-6- | A | A′ | A | A′ |
| (trifluoromethyl)phenyl]methoxy] | | | | |
| pyrimidin-2-yl]morpholin-2- | | | | |
| yl]methanol | | | | |
| [(2R)-4-[5-[[2-chloro-6- | A | A′ | A | A′ |
| (trifluoromethyl)phenyl]methoxy] | | | | |
| pyrimidin-2-yl]morpholin-2- | | | | |
| yl]methanol | | | | |
| 1-[5-[(2,6-dichlorophenyl)methoxy]- | B | B′ | C | A′ |
| 2-pyridyl]ethane-1,2-diol | | | | |
| (4R)-1-[5-[(2,6- | B | A′ | B | A′ |
| dichlorophenyl)methoxy]-2-pyridyl]- | | | | |
| 4-(hydroxymethyl)imidazolidin-2- | | | | |
| one | | | | |
| 4-[5-[[2-methyl-6- | A | A′ | A | A′ |
| (trifluoromethyl)phenyl]methoxy] | | | | |
| pyrimidin-2-yl]piperazin-2-one | | | | |
| 1-[5-[[2-methyl-6- | A | A′ | A | A′ |
| (trifluoromethyl)phenyl]methoxy] | | | | |
| pyrimidin-2-yl]benzotriazole | | | | |
| [1-[5-[[2-ethyl-6- | A | A′ | A | A′ |
| (trifluoromethyl)phenyl]methoxy] | | | | |
| pyrimidin-2-yl]pyrazol-3-yl]methanol | | | | |
| [1-[5-[[2-ethyl-6- | A | A′ | A | A′ |
| (trifluoromethyl)phenyl]methoxy] | | | | |
| pyrimidin-2-yl]imidazol-4-yl]methanol | | | | |
| (2S)-N-[5-[(2,6- | C | B′ | C | B |
| dichlorophenyl)methoxy]-2-pyridyl]- | | | | |
| 2-formamido-3-hydroxy- | | | | |
| propanamide | | | | |
| (5R)-3-[5-[(2,6- | C | B′ | C | A′ |
| dichlorophenyl)methoxy]-2-pyridyl]- | | | | |
| 5-(hydroxymethyl)imidazolidine- | | | | |
| 2,4-dione | | | | |
| 3-[5-[(2,6-dichlorophenyl)methoxy]- | B | B′ | B | B′ |
| 2-pyridyl]-5-methylene- | | | | |
| imidazolidine-2,4-dione | | | | |
| [4-[4-[[2-ethyl-6- | A | A′ | A | A′ |
| (trifluoromethyl)phenyl]methoxy] | | | | |
| phenyl]morpholin-2-yl]methanol | | | | |
| [4-[5-[(2,6- | B | A′ | B | A′ |
| dichlorophenyl)methoxy]pyrimidin- | | | | |
| 2-yl]piperazin-2-yl]methanol | | | | |
| [4-[5-[(2,6- | B | A′ | B | A′ |
| dichlorophenyl)methoxy]pyrimidin- | | | | |
| 2-yl]-6,6-dimethyl-morpholin-2- | | | | |
| yl]methanol | | | | |
| methyl 4-[5-[(2,6- | C | B′ | C | A′ |
| dichlorophenyl)methoxy]pyrimidin- | | | | |
| 2-yl]-6-oxo-piperazine-2-carboxylate | | | | |
| 4-[5-[(2,6- | B | A′ | B | A′ |
| dichlorophenyl)methoxy]pyrimidin- | | | | |
| 2-yl]morpholine | | | | |
| 3-[5-[(2,6- | C | B′ | C | A′ |
| dichlorophenyl)methoxy]pyrimidin- | | | | |
| 2-yl]-5-isopropyl-imidazolidine-2,4- | | | | |
| dione | | | | |
| 5,5-dimethyl-3-[5-[[2-methyl-6- | B | A′ | B | A′ |
| (trifluoromethyl)phenyl]methoxy] | | | | |
| pyrimidin-2-yl]imidazolidine-2,4-dione | | | | |
| 8-[5-[[2-methyl-6- | A | A′ | A | A′ |
| (trifluoromethyl)phenyl]methoxy] | | | | |
| pyrimidin-2-yl]-3,4,6,7,9,9a- | | | | |
| hexahydro-1H-pyrazino[2,1- | | | | |
| c][1,4]oxazine | | | | |
| 4-[5-[(2,6- | B | A′ | B | A′ |
| dichlorophenyl)methoxy]pyrimidin- | | | | |
| 2-yl]-6-(hydroxymethyl)piperazin-2- | | | | |
| one | | | | |
| [4-[5-[(2,6- | B | A′ | A | A′ |
| dichlorophenyl)methoxy]pyrimidin- | | | | |
| 2-yl]morpholin-2-yl]methanamine | | | | |
| 4-[5-[(2,6- | A | A′ | A | A′ |
| dichlorophenyl)methoxy]pyrimidin- | | | | |
| 2-yl]-N-methyl-piperazine-1- | | | | |
| carboxamide | | | | |
| (2R)-2-acetamido-N-[5-[(2,6- | C | C′ | C | C′ |
| dichlorophenyl)methoxy]-2-pyridyl]- | | | | |
| 3-hydroxy-propanamide | | | | |
| 1-methyl-3-[5-[[2-methyl-6- | C | C′ | C | B′ |
| (trifluoromethyl)phenyl]methoxy] | | | | |
| pyrimidin-2-yl]imidazolidine-2,4-dione | | | | |
| (4S)-1-[5-[(2,6- | A | A′ | A | A′ |
| dichlorophenyl)methoxy]-2-pyridyl]- | | | | |
| 4-(hydroxymethyl)imidazolidin-2- | | | | |
| one | | | | |
| (4S)-4-(hydroxymethyl)-1-[5-[[2- | A | A′ | A | A′ |
| methyl-6- | | | | |
| (trifluoromethyl)phenyl]methoxy]-2- | | | | |
| pyridyl]imidazolidin-2-one | | | | |
| 3-[5-[(2,6- | C | A′ | B | A′ |
| dichlorophenyl)methoxy]pyrimidin- | | | | |
| 2-yl]-1,3-diazaspiro[4.4]nonane-2,4- | | | | |
| dione | | | | |
| 1-butyl-3-[5-[(2,6- | C | C′ | C | — |
| dichlorophenyl)methoxy]pyrimidin- | | | | |
| 2-yl]imidazolidine-2,4-dione | | | | |
| 4-[5-[[2-methyl-6- | B | A′ | B | A′ |
| (trifluoromethyl)phenyl]methoxy] | | | | |
| pyrimidin-2-yl]-1-oxido- | | | | |
| thiomorpholin-1-ium | | | | |
| 4-[5-[(2,6- | B | A′ | B | A′ |
| dichlorophenyl)methoxy]pyrimidin- | | | | |
| 2-yl]-1,4-diazepane-1-carboxamide | | | | |
| 1-[4-[5-[(2,6- | B | A′ | B | A′ |
| dichlorophenyl)methoxy]pyrimidin- | | | | |
| 2-yl]piperazin-1-yl]-2-hydroxy- | | | | |
| ethanone | | | | |
| [rac-(2R,6S)-4-[5-[(2,6- | B | A′ | B | A′ |
| dichlorophenyl)methoxy]pyrimidin- | | | | |
| 2-yl]-6-(trifluoromethyl)morpholin- | | | | |
| 2-yl]methanol | | | | |
| [rac-(2S,5S)-4-[5-[(2,6- | B | A′ | B | A′ |
| dichlorophenyl)methoxy]pyrimidin- | | | | |
| 2-yl]-5-methyl-morpholin-2- | | | | |
| yl]methanol | | | | |
| [rac-(2R,5S)-4-[5-[(2,6- | A | A′ | A | A′ |
| dichlorophenyl)methoxy]pyrimidin- | | | | |
| 2-yl]-5-methyl-morpholin-2- | | | | |
| yl]methanol | | | | |
| (3aR,6aS)-2-[5-[(2,6- | B | B′ | B | A′ |
| dichlorophenyl)methoxy]pyrimidin- | | | | |
| 2-yl]-1,3,3a,4,6,6a- | | | | |
| hexahydropyrrolo[3,4-c]pyrrole-5- | | | | |
| carboxamide | | | | |
| 4-[5-[(2,6- | A | A′ | A | A′ |
| dichlorophenyl)methoxy]pyrimidin- | | | | |
| 2-yl]-2-methyl-piperazine-1- | | | | |
| carboxamide | | | | |
| 4-[5-[(2,6- | C | A′ | B | A′ |
| dichlorophenyl)methoxy]pyrimidin- | | | | |
| 2-yl]piperazine-1-sulfonamide | | | | |
| [rac-(2S,6S)-4-[5-[(2,6- | B | B′ | A | A′ |
| dichlorophenyl)methoxy]pyrimidin- | | | | |
| 2-yl]-6-(trifluoromethyl)morpholin- | | | | |
| 2-yl]methanol | | | | |
| 4-[5-[(2,6- | A | A′ | A | B′ |
| dichlorophenyl)methoxy]pyrimidin- | | | | |
| 2-yl]-N,N-dimethyl-piperazine-1- | | | | |
| carboxamide | | | | |
| 2-[4-[5-[(2,6- | B | B′ | B | B′ |
| dichlorophenyl)methoxy]pyrimidin- | | | | |
| 2-yl]piperazin-1-yl]acetamide | | | | |
| [1-[4-[[2-chloro-6- | B | A′ | A | A′ |
| (trifluoromethyl)phenyl]methoxy] | | | | |
| phenyl]-1,2,4-triazol-3-yl]methanol | | | | |
| rac-(1R)-1-[rac-(2S)-4-[5-[(2,6- | C | A′ | B | A′ |
| dichlorophenyl)methoxy]pyrimidin- | | | | |
| 2-yl]morpholin-2-yl]ethanol | | | | |
| 1-[4-[[2-methyl-6- | B | A′ | B | A′ |
| (trifluoromethyl)phenyl]methoxy] | | | | |
| phenyl]-1,2,4-triazole-3-carboxylic | | | | |
| acid | | | | |
| N-[[4-[5-[(2,6- | B | A′ | A | A′ |
| dichlorophenyl)methoxy]pyrimidin- | | | | |
| 2-yl]morpholin-2- | | | | |
| yl]methyl]acetamide | | | | |
| 4-[5-[(2,6- | A | A′ | A | A′ |
| dichlorophenyl)methoxy]pyrimidin- | | | | |
| 2-yl]-2-(hydroxymethyl)piperazine- | | | | |
| 1-carboxamide | | | | |
| methyl 1-[4-[[2-methyl-6- | B | A′ | A | A′ |
| (trifluoromethyl)phenyl]methoxy] | | | | |
| phenyl]-1,2,4-triazole-3-carboxylate | | | | |
| 1-[5-[(2,6- | A | A′ | A | A′ |
| dichlorophenyl)methoxy]pyrimidin- | | | | |
| 2-yl]piperidin-4-ol | | | | |
| 1-[5-[(2,6- | B | A′ | A | A′ |
| dichlorophenyl)methoxy]pyrimidin- | | | | |
| 2-yl]-4-phenyl-piperidin-4-ol | | | | |
| 1-[5-[(2,6- | B | A′ | A | A′ |
| dichlorophenyl)methoxy]pyrimidin- | | | | |
| 2-yl]piperidin-3-ol | | | | |
| rel-(3aR,6aS)-5-[5-[(2,6- | B | A′ | B | A′ |
| dichlorophenyl)methoxy]pyrimidin- | | | | |
| 2-yl]-1,3,3a,4,6,6a- | | | | |
| hexahydrofuro[3,4-c]pyrrole | | | | |
| 2-[2-[4-[5-[(2,6- | A | A′ | A | A′ |
| dichlorophenyl)methoxy]pyrimidin- | | | | |
| 2-yl]piperazin-1-yl]ethoxy]ethanol | | | | |
| 4-[[5-[(2,6- | A | A′ | A | A′ |
| dichlorophenyl)methoxy]pyrimidin- | | | | |
| 2-yl]-methyl-amino]butan-1-ol | | | | |
| 1-[5-[(2,6- | B | A′ | B | A′ |
| dichlorophenyl)methoxy]pyrimidin- | | | | |
| 2-yl]-N-methyl-piperidine-4- | | | | |
| carboxamide | | | | |
| [1-[5-[(2,6- | B | A′ | B | A′ |
| dichlorophenyl)methoxy]pyrimidin- | | | | |
| 2-yl]-5-methyl-pyrrolidin-3- | | | | |
| yl]methanol | | | | |
| N-[(3S)-1-[5-[(2,6- | B | A′ | B | A′ |
| dichlorophenyl)methoxy]pyrimidin- | | | | |
| 2-yl]pyrrolidin-3-yl]acetamide | | | | |
| 1-[1-[5-[(2,6- | B | A′ | B | A′ |
| dichlorophenyl)methoxy]pyrimidin- | | | | |
| 2-yl]pyrrolidin-3-yl]ethanol | | | | |
| (3S)-1-[5-[(2,6- | B | A′ | B | A′ |
| dichlorophenyl)methoxy]pyrimidin- | | | | |
| 2-yl]pyrrolidin-3-ol | | | | |
| 1-[5-[(2,6- | B | A′ | B | B′ |
| dichlorophenyl)methoxy]pyrimidin- | | | | |
| 2-yl]azetidine-3-carboxamide | | | | |
| 2-[5-[(2,6- | B | A′ | A | A′ |
| dichlorophenyl)methoxy]pyrimidin- | | | | |
| 2-yl]-6-oxa-2-azaspiro[3.5]nonane | | | | |
| 1-[5-[(2,6- | B | A′ | B | A′ |
| dichlorophenyl)methoxy]pyrimidin- | | | | |
| 2-yl]pyrrolidine-3-carboxamide | | | | |
| 2-[4-[5-[(2,6- | B | A′ | B | A′ |
| dichlorophenyl)methoxy]pyrimidin- | | | | |
| 2-yl]piperazin-1-yl]ethanol | | | | |
| 1-[5-[(2,6- | A | A′ | A | A′ |
| dichlorophenyl)methoxy]pyrimidin- | | | | |
| 2-yl]piperidine-3-carboxamide | | | | |
| 5-[(2,6-dichlorophenyl)methoxy]-2- | B | B′ | B | A′ |
| (3-methoxypyrrolidin-1- | | | | |
| yl)pyrimidine | | | | |
| 1-[5-[(2,6- | A | A′ | A | A′ |
| dichlorophenyl)methoxy]pyrimidin- | | | | |
| 2-yl]-3-methyl-piperidin-4-ol | | | | |
| 3-[[5-[(2,6- | C | B′ | B | B′ |
| dichlorophenyl)methoxy]pyrimidin- | | | | |
| 2-yl]-methyl-amino]propan-1-ol | | | | |
| 7-[5-[(2,6- | B | A′ | A | A′ |
| dichlorophenyl)methoxy]pyrimidin- | | | | |
| 2-yl]-1-oxa-7-azaspiro[3.4]octane | | | | |
| [1-[5-[(2,6- | B | A′ | B | A′ |
| dichlorophenyl)methoxy]pyrimidin- | | | | |
| 2-yl]-3-piperidyl]methanol | | | | |
| 2-[1-[5-[(2,6- | B | B′ | A | A′ |
| dichlorophenyl)methoxy]pyrimidin- | | | | |
| 2-yl]-4-piperidyl]ethanol | | | | |
| 1-benzyl-3-(5-benzyloxypyrimidin- | B | A′ | A | A′ |
| 2-yl)imidazolidine-2,4-dione | | | | |
| 6-(hydroxymethyl)-4-[5-[[2-methyl- | B | A′ | B | A′ |
| 6- | | | | |
| (trifluoromethyl)phenyl]methoxy] | | | | |
| pyrimidin-2-yl]morpholin-3-one | | | | |
| 5-[[2-chloro-6- | B | A′ | A | A′ |
| (trifluoromethyl)phenyl]methoxy]-2- | | | | |
| (1,2,4-triazol-1-yl)pyrimidine | | | | |
| 5-[(2,6-dichlorophenyl)methoxy]-2- | B | A′ | B | A′ |
| [4-(2-pyridyl)triazol-2-yl]pyrimidine | | | | |
| 5-[(2,6-dichlorophenyl)methoxy]-2- | A | A′ | A | A′ |
| [4-(2-pyridyl)triazol-1-yl]pyrimidine | | | | |
| 1-benzyl-3-[5-[[2-methyl-6- | A | A′ | A | A′ |
| (trifluoromethyl)phenyl]methoxy] | | | | |
| pyrimidin-2-yl]imidazolidine-2,4-dione | | | | |
| 4-[5-[(2,6- | B | A′ | B | A′ |
| dichlorophenyl)methoxy]pyrimidin- | | | | |
| 2-yl]-1-(2-hydroxyethyl)piperazin-2- | | | | |
| one | | | | |
| 1-[4-[5-[(2,6- | C | B′ | C | A′ |
| dichlorophenyl)methoxy]pyrimidin- | | | | |
| 2-yl]morpholin-2-yl]-N,N-dimethyl- | | | | |
| methanamine | | | | |
| 1-[4-[[2-ethyl-6- | B | A′ | A | A′ |
| (trifluoromethyl)phenyl]methoxy] | | | | |
| phenyl]-4- | | | | |
| (hydroxymethyl)imidazolidin-2-one | | | | |
| 5-[[2-methyl-6- | B | A′ | B | A′ |
| (trifluoromethyl)phenyl]methoxy]-2- | | | | |
| (1,2,4-triazol-4-yl)pyrimidine | | | | |
| 1-[4-[[2-methyl-6- | A | A′ | A | B′ |
| (trifluoromethyl)phenyl]methoxy] | | | | |
| phenyl]-1,2,4-triazole-3-carboxamide | | | | |
| [4-[5-[[2-methyl-6- | A | A′ | A | A′ |
| (trifluoromethyl)phenyl]methoxy] | | | | |
| pyrimidin-2-yl]morpholin-2- | | | | |
| yl]methanamine | | | | |
| [1-[5-[(2,6- | A | A′ | A | A′ |
| dichlorophenyl)methoxy]pyrimidin- | | | | |
| 2-yl]-3-piperidyl]methanamine | | | | |
| N-[[1-[5-[(2,6- | B | B′ | B | B′ |
| dichlorophenyl)methoxy]pyrimidin- | | | | |
| 2-yl]-3-piperidyl]methyl]formamide | | | | |
| [1-[5-[(2,6- | A | A′ | A | A′ |
| dichlorophenyl)methoxy]pyrimidin- | | | | |
| 2-yl]-4-fluoro-4-piperidyl]methanol | | | | |
| 4-[5-[(2,6- | B | A′ | B | A′ |
| dichlorophenyl)methoxy]pyrimidin- | | | | |
| 2-yl]-6-hydroxy-1,4-diazepane-1- | | | | |
| carboxamide | | | | |
| N-[[4-[5-[(2,6- | B | B′ | B | A′ |
| dichlorophenyl)methoxy]pyrimidin- | | | | |
| 2-yl]morpholin-2-yl]methyl]-2,2,2- | | | | |
| trifluoro-acetamide | | | | |
| [1-[5-[[2-methyl-6- | B | A′ | A | A′ |
| (trifluoromethyl)phenyl]methoxy] | | | | |
| pyrimidin-2-yl]-1,2,4-triazol-3- | | | | |
| yl]methanol | | | | |
| [1-[5-[[2-chloro-6- | B | A′ | A | A′ |
| (trifluoromethyl)phenyl]methoxy] | | | | |
| pyrimidin-2-yl]-1,2,4-triazol-3- | | | | |
| yl]methanol | | | | |
| [1-[5-[[2-methyl-6- | A | A′ | A | A′ |
| (trifluoromethyl)phenyl]methoxy]-2- | | | | |
| pyridyl]-1,2,4-triazol-3-yl]methanol | | | | |
| [1-[5-[[2-chloro-6- | A | A′ | A | A′ |
| (trifluoromethyl)phenyl]methoxy]-2- | | | | |
| pyridyl]-1,2,4-triazol-3-yl]methanol | | | | |
| 1-[5-[(2,6- | A | B′ | B | A′ |
| dichlorophenyl)methoxy]pyrimidin- | | | | |
| 2-yl]pyrazole-3-carbonitrile | | | | |
| 1-[5-[(2,6- | B | C′ | B | A′ |
| dichlorophenyl)methoxy]pyrimidin- | | | | |
| 2-yl]-1,2,4-triazole-3-carbonitrile | | | | |
| 1-methyl-3-[4-[[2-methyl-6- | B | A′ | A | A′ |
| (trifluoromethyl)phenyl]methoxy] | | | | |
| phenyl]imidazolidine-2,4-dione | | | | |
| 5,5-dimethyl-3-[4-[[2-methyl-6- | A | A′ | A | A′ |
| (trifluoromethyl)phenyl]methoxy] | | | | |
| phenyl]imidazolidine-2,4-dione | | | | |
| 1-[5-[(2,6- | A | B′ | A | A′ |
| dichlorophenyl)methoxy]pyrimidin- | | | | |
| 2-yl]pyrazole-3-carboxamide | | | | |
| 1-[5-[(2,6- | C | — | B | C′ |
| dichlorophenyl)methoxy]pyrimidin- | | | | |
| 2-yl]-1,2,4-triazole-3-carboxamide | | | | |
| [1-[4-[[2-(1,1-difluoroethyl)-6- | B | B′ | B | B′ |
| methyl- | | | | |
| phenyl]methoxy]phenyl]triazol-4- | | | | |
| yl]methanol | | | | |
| [1-[5-[[2-methyl-6- | A | A′ | A | A′ |
| (trifluoromethyl)phenyl]methoxy] | | | | |
| pyrimidin-2-yl]pyrrolidin-3- | | | | |
| yl]methanol | | | | |
| 1-[5-[[2-methyl-6- | B | A′ | A | A′ |
| (trifluoromethyl)phenyl]methoxy] | | | | |
| pyrimidin-2-yl]pyrrolidine-3- | | | | |
| carboxamide | | | | |
| 1-[5-[[2-chloro-6- | A | A′ | A | A′ |
| (trifluoromethyl)phenyl]methoxy] | | | | |
| pyrimidin-2-yl]pyrazole-3- | | | | |
| carboxamide | | | | |
| [4-(hydroxymethyl)-1-[5-[[2-methyl- | A | A′ | A | A′ |
| 6- | | | | |
| (trifluoromethyl)phenyl]methoxy] | | | | |
| pyrimidin-2-yl]-4-piperidyl]methanol | | | | |
| N-[[4-[5-[(2,6- | B | B′ | B | A′ |
| dichlorophenyl)methoxy]pyrimidin- | | | | |
| 2-yl]morpholin-2- | | | | |
| yl]methyl]methanesulfonamide | | | | |
| [4-[5-[(2,6- | B | B′ | B | A′ |
| dichlorophenyl)methoxy]pyrimidin- | | | | |
| 2-yl]morpholin-2-yl]methylurea | | | | |
| [(2S)-4-[5-[(2,6- | B | A′ | A | A′ |
| dichlorophenyl)methoxy]pyrimidin- | | | | |
| 2-yl]morpholin-2-yl]methanamine | | | | |
| [(2R)-4-[5-[(2,6- | B | A′ | A | B′ |
| dichlorophenyl)methoxy]pyrimidin- | | | | |
| 2-yl]morpholin-2-yl]methanamine | | | | |
| rac-(3S,4S)-1-[5-[(2,6- | B | A′ | A | A′ |
| dichlorophenyl)methoxy]pyrimidin- | | | | |
| 2-yl]-3-(hydroxymethyl)piperidin-4- | | | | |
| ol | | | | |
| 4-[methyl-[5-[[2-methyl-6- | A | A′ | A | A′ |
| (trifluoromethyl)phenyl]methoxy] | | | | |
| pyrimidin-2-yl]amino]butan-1-ol | | | | |
| 1-[5-[[2-methyl-6- | A | A′ | A | A′ |
| (trifluoromethyl)phenyl]methoxy] | | | | |
| pyrimidin-2-yl]piperidin-4-ol | | | | |
| 2-[2-[4-[5-[[2-methyl-6- | B | A′ | B | A′ |
| (trifluoromethyl)phenyl]methoxy] | | | | |
| pyrimidin-2-yl]piperazin-1- | | | | |
| yl]ethoxy]ethanol | | | | |
| (3S)-1-[5-[[2-methyl-6- | A | A′ | A | A′ |
| (trifluoromethyl)phenyl]methoxy] | | | | |
| pyrimidin-2-yl]piperidin-3-ol | | | | |
| (3R)-1-[5-[2-methyl-6- | B | A′ | A | A′ |
| (trifluoromethyl)phenyl]methoxy] | | | | |
| pyrimidin-2-yl]piperidin-3-ol | | | | |
| 2-(hydroxymethyl)-4-[5-[[2-methyl- | A | A′ | A | A′ |
| 6- | | | | |
| (trifluoromethyl)phenyl]methoxy] | | | | |
| pyrimidin-2-yl]piperazine-1- | | | | |
| carboxamide | | | | |
| 5-[(2,6-dichlorophenyl)methoxy]-2- | C | — | C | — |
| (1H-tetrazol-5-yl)pyrimidine | | | | |
| 3-[5-[2-(2,6- | C | — | C | — |
| dichlorophenyl)ethyl]pyrimidin-2- | | | | |
| yl]-5-(hydroxymethyl)oxazolidin-2- | | | | |
| one | | | | |
| 1,1-dimethyl-3-[5-[[2-methyl-6- | B | C′ | B | A′ |
| (trifluoromethyl)phenyl]methoxy] | | | | |
| pyrimidin-2-yl]urea | | | | |
| 1-[5-[[2-chloro-6- | C | — | B | C′ |
| (trifluoromethyl)phenyl]methoxy] | | | | |
| pyrimidin-2-yl]hexahydropyrimidine- | | | | |
| 2,4-dione | | | | |
| [(2S)-4-[5-[[2-methyl-6- | A | A′ | A | A′ |
| (trifluoromethyl)phenyl]methoxy] | | | | |
| pyrimidin-2-yl]morpholin-2- | | | | |
| yl]methanamine | | | | |
| [(2R)-4-[5-[[2-methyl-6- | A | A′ | A | A′ |
| (trifluoromethyl)phenyl]methoxy] | | | | |
| pyrimidin-2-yl]morpholin-2- | | | | |
| yl]methanamine | | | | |
| [3-[5-[(2,6- | B | A′ | A | A′ |
| dichlorophenyl)methoxy]pyrimidin- | | | | |
| 2-yl]isoxazol-5-yl]methanol | | | | |
| 5-[[2-methyl-6- | A | A′ | A | A′ |
| (trifluoromethyl)phenyl]methoxy]-2- | | | | |
| [4-(2-pyridyl)triazol-2-yl]pyrimidine | | | | |
| (2S)-2-methyl-4-[5-[[2-methyl-6- | A | A′ | A | A′ |
| (trifluoromethyl)phenyl]methoxy] | | | | |
| pyrimidin-2-yl]piperazine-1- | | | | |
| carboxamide | | | | |
| 5-[[2-methyl-6- | A | B′ | A | A′ |
| (trifluoromethyl)phenyl]methoxy]-2- | | | | |
| [4-(2-pyridyl)triazol-1-yl]pyrimidine | | | | |
| N-[[(2S)-4-[5-[[2-methyl-6- | B | A′ | A | A′ |
| (trifluoromethyl)phenyl]methoxy] | | | | |
| pyrimidin-2-yl]morpholin-2- | | | | |
| yl]methyl]acetamide | | | | |
| 5-[[2-chloro-6- | A | A′ | A | A′ |
| (trifluoromethyl)phenyl]methoxy]-2- | | | | |
| [4-(2-pyridyl)triazol-2-yl]pyrimidine | | | | |
| 5-[[2-chloro-6- | A | A′ | A | A′ |
| (trifluoromethyl)phenyl]methoxy]-2- | | | | |
| [4-(2-pyridyl)triazol-1-yl]pyrimidine | | | | |
| N-[[(2R)-4-[5-[[2-methyl-6- | B | A′ | A | A′ |
| (trifluoromethyl)phenyl]methoxy] | | | | |
| pyrimidin-2-yl]morpholin-2- | | | | |
| yl]methyl]acetamide | | | | |
| [1-[4-[(2,6- | B | A′ | B | B′ |
| dichlorophenyl)methoxy]phenyl] | | | | |
| pyrazol-3-yl]methanamine | | | | |
| 4-[5-[[2-(1,1-difluoroethyl)-6- | A | A′ | A | A′ |
| methyl-phenyl]methoxy]pyrimidin- | | | | |
| 2-yl]piperazine-1-carboxamide | | | | |
| [1-[5-[(2,6- | B | A′ | B | A′ |
| dichlorophenyl)methoxy]-2- | | | | |
| pyridyl]triazol-4-yl]methanamine | | | | |
| [5-[4-[(2,6- | B | B′ | B | B′ |
| dichlorophenyl)methoxy]phenyl]- | | | | |
| 1,3,4-oxadiazol-2-yl]methanamine | | | | |
| [1-[4-[(2,6- | B | B′ | B | B′ |
| dichlorophenyl)methoxy]phenyl] | | | | |
| imidazol-4-yl]methanamine | | | | |
| 1-[4-[5-[(2,6- | B | B′ | B | B′ |
| dichlorophenyl)methoxy]pyrimidin- | | | | |
| 2-yl]morpholin-2-yl]-N-methyl- | | | | |
| methanamine | | | | |
| 5-[(2,6-dichlorophenyl)methoxy]-N- | B | C′ | B | C′ |
| methyl-N-(morpholin-2- | | | | |
| ylmethyl)pyrimidin-2-amine | | | | |
| 3-[(4-methoxyphenyl)methyl]-1-[4- | B | B′ | B | B′ |
| [[2-methyl-6- | | | | |
| (trifluoromethyl)phenyl]methoxy] | | | | |
| phenyl]imidazolidine-2,4-dione | | | | |
| N-methyl-4-[5-[[2-methyl-6- | A | A′ | A | A′ |
| (trifluoromethyl)phenyl]methoxy] | | | | |
| pyrimidin-2-yl]piperazine-1- | | | | |
| carboxamide | | | | |
| 1-[5-[[2-(1,1-difluoroethyl)-6- | B | A′ | B | A′ |
| methyl-phenyl]methoxy]-2-pyridyl]- | | | | |
| 4-(hydroxymethyl)imidazolidin-2- | | | | |
| one | | | | |
| [3-[5-[(2,6- | B | A′ | A | A′ |
| dichlorophenyl)methoxy]-2- | | | | |
| pyridyl]isoxazol-5-yl]methanamine | | | | |
| 5-isopropyl-3-[4-[[2-methyl-6- | B | A′ | A | A′ |
| (trifluoromethyl)phenyl]methoxy] | | | | |
| phenyl]imidazolidine-2,4-dione | | | | |
| 3-[4-[[2-methyl-6- | B | B′ | A | A′ |
| (trifluoromethyl)phenyl]methoxy] | | | | |
| phenyl]-1,3-diazaspiro[4.4]nonane-2,4- | | | | |
| dione | | | | |
| 1-butyl-3-[4-[[2-methyl-6- | B | B′ | B | B′ |
| (trifluoromethyl)phenyl]methoxy] | | | | |
| phenyl]imidazolidine-2,4-dione | | | | |
| 3-methyl-1-[5-[[2-methyl-6- | A | A′ | A | A′ |
| (trifluoromethyl)phenyl]methoxy]-2- | | | | |
| pyridyl]imidazolidine-2,4-dione | | | | |
| 1-[5-[[2-chloro-6- | A | A′ | A | A′ |
| (trifluoromethyl)phenyl]methoxy]-2- | | | | |
| pyridyl]-3-methyl-imidazolidine-2,4- | | | | |
| dione | | | | |
| 3-methyl-1-[5-[[2-methyl-6- | B | A′ | B | A′ |
| (trifluoromethyl)phenyl]methoxy] | | | | |
| pyrimidin-2-yl]imidazolidine-2,4-dione | | | | |
| 3-(hydroxymethyl)-1-[5-[[2-methyl- | B | A′ | B | A′ |
| 6- | | | | |
| (trifluoromethyl)phenyl]methoxy] | | | | |
| pyrimidin-2-yl]imidazolidine-2,4-dione | | | | |
| [5-[5-[[2-chloro-6- | B | A′ | A | A′ |
| (trifluoromethyl)phenyl]methoxy] | | | | |
| pyrimidin-2-yl]-1H-pyrazol-3- | | | | |
| yl]methanol | | | | |
| 5-[[2-chloro-6- | B | A′ | B | A′ |
| (trifluoromethyl)phenyl]methoxy]-2- | | | | |
| (3,3-difluoropyrrolidin-1- | | | | |
| yl)pyrimidine | | | | |
| 1-[5-[[2-chloro-6- | A | A′ | A | A′ |
| (trifluoromethyl)phenyl]methoxy] | | | | |
| pyrimidin-2-yl]-4-methyl-piperidin-4- | | | | |
| ol | | | | |
| [5-[4-[[2-chloro-6- | A | A′ | A | A′ |
| (trifluoromethyl)phenyl]methoxy] | | | | |
| phenyl]-1,3,4-oxadiazol-2-yl]methanol | | | | |
| (3R)-1-[5-[[2-methyl-6- | B | A′ | B | A′ |
| (trifluoromethyl)phenyl]methoxy] | | | | |
| pyrimidin-2-yl]pyrrolidine-3- | | | | |
| carboxamide | | | | |
| (3R)-1-[5-[[2-chloro-6- | B | A′ | B | A′ |
| (trifluoromethyl)phenyl]methoxy] | | | | |
| pyrimidin-2-yl]pyrrolidine-3- | | | | |
| carboxamide | | | | |
| 3,3-dimethyl-1-[5-[[2-methyl-6- | A | A′ | A | A′ |
| (trifluoromethyl)phenyl]methoxy] | | | | |
| pyrimidin-2-yl]piperidin-4-ol | | | | |
| 1-[5-[[2-methyl-6- | B | A′ | A | A′ |
| (trifluoromethyl)phenyl]methoxy] | | | | |
| pyrimidin-2-yl]piperidin-4-amine | | | | |
| 3-[5-[[2-methyl-6- | B | A′ | B | A′ |
| (trifluoromethyl)phenyl]methoxy] | | | | |
| pyrimidin-2-yl]-3- | | | | |
| azabicyclo[3.1.1]heptan-6-ol | | | | |
| 1-[5-[[2-methyl-6- | B | A′ | B | A′ |
| (trifluoromethyl)phenyl]methoxy] | | | | |
| pyrimidin-2-yl]imidazolidine-2,4-dione | | | | |
| [5-[5-[(2,6- | B | B′ | B | A′ |
| dichlorophenyl)methoxy]pyrimidin- | | | | |
| 2-yl]-1H-pyrazol-3-yl]methanol | | | | |
| [2-[5-[[2-methyl-6- | A | A′ | A | A′ |
| (trifluoromethyl)phenyl]methoxy]-2- | | | | |
| pyridyl]triazol-4-yl]methanol | | | | |
| [1-[5-[[2-methyl-6- | A | A′ | A | A′ |
| (trifluoromethyl)phenyl]methoxy]-2- | | | | |
| pyridyl]triazol-4-yl]methanol | | | | |
| 3-[5-[[2-chloro-6- | B | C′ | B | A′ |
| (trifluoromethyl)phenyl]methoxy]-2- | | | | |
| pyridyl]-1-methyl-imidazolidine-2,4- | | | | |
| dione | | | | |
| 5,5-dimethyl-3-[5-[[2-methyl-6- | B | B′ | B | A′ |
| (trifluoromethyl)phenyl]methoxy]-2- | | | | |
| pyridyl]imidazolidine-2,4-dione | | | | |
| 3-[5-[[2-chloro-6- | B | B′ | B | A′ |
| (trifluoromethyl)phenyl]methoxy]-2- | | | | |
| pyridyl]-5,5-dimethyl-imidazolidine- | | | | |
| 2,4-dione | | | | |
| [3-[5-[[2-methyl-6- | B | A′ | A | A′ |
| (trifluoromethyl)phenyl]methoxy] | | | | |
| pyrimidin-2-yl]isoxazol-5-yl]methanol | | | | |
| [1-[5-[(2,6-dichlorophenyl)-difluoro- | C | — | C | — |
| methoxy]pyrimidin-2-yl]pyrazol-3- | | | | |
| yl]methanol | | | | |
| 1-[5-[[2-methyl-6- | B | A′ | A | A′ |
| (trifluoromethyl)phenyl]methoxy] | | | | |
| pyrimidin-2-yl]-4-(2-pyridyl)piperidin- | | | | |
| 4-ol | | | | |
| 1-[5-[[2-methyl-6- | A | A′ | A | A′ |
| (trifluoromethyl)phenyl]methoxy] | | | | |
| pyrimidin-2-yl]-4-(3-pyridyl)piperidin- | | | | |
| 4-ol | | | | |
| (3S)-1-[5-[[2-methyl-6- | B | A′ | B | A′ |
| (trifluoromethyl)phenyl]methoxy] | | | | |
| pyrimidin-2-yl]pyrrolidine-3- | | | | |
| carboxamide | | | | |
| (3S)-1-[5-[[2-chloro-6- | B | A′ | B | A′ |
| (trifluoromethyl)phenyl]methoxy] | | | | |
| pyrimidin-2-yl]pyrrolidine-3- | | | | |
| carboxamide | | | | |
| [3-[5-[[2-chloro-6- | B | A′ | A | A′ |
| (trifluoromethyl)phenyl]methoxy] | | | | |
| pyrimidin-2-yl]isoxazol-5-yl]methanol | | | | |
| [1-[4-[[2-methyl-6- | B | A′ | A | A′ |
| (trifluoromethyl)phenyl]methoxy] | | | | |
| phenyl]-1,2,4-triazol-3-yl]methanamine | | | | |
| 1-[5-[(2,6- | A | A′ | A | A′ |
| dichlorophenyl)methoxy]pyrimidin- | | | | |
| 2-yl]-4-(2-hydroxyethyl)piperidin-4- | | | | |
| ol | | | | |
| [1-[5-[[2-chloro-6- | B | B′ | A | A′ |
| (trifluoromethoxy)phenyl]methoxy] | | | | |
| pyrimidin-2-yl]pyrazol-3-yl]methanol | | | | |
| [1-[5-[[2-chloro-6- | B | B′ | B | A′ |
| (difluoromethoxy)phenyl]methoxy] | | | | |
| pyrimidin-2-yl]imidazol-4- | | | | |
| yl]methanol | | | | |
| 4-(2-hydroxyethyl)-1-[5-[[2-methyl- | A | A′ | A | A′ |
| 6- | | | | |
| (trifluoromethyl)phenyl]methoxy] | | | | |
| pyrimidin-2-yl]piperidin-4-ol | | | | |
| [2-[5-[[2-chloro-6- | B | A | A | A′ |
| (trifluoromethyl)phenyl]methoxy] | | | | |
| pyrimidin-2-yl]triazol-4-yl]methanol | | | | |
| 1-[5-[[2-chloro-6- | A | A′ | A | A′ |
| (trifluoromethyl)phenyl]methoxy]-2- | | | | |
| pyridyl]imidazolidine-2,4-dione | | | | |
| 8-[5-[[2-methyl-6- | A | A′ | A | A′ |
| (trifluoromethyl)phenyl]methoxy] | | | | |
| pyrimidin-2-yl]-6,7,9,9a-tetrahydro- | | | | |
| 1H-pyrazino[2,1-c][1,4]oxazin-4-one | | | | |
| rac-(3S,5R)-1-[5-[[2-methyl-6- | B | A′ | B | A′ |
| (trifluoromethyl)phenyl]methoxy] | | | | |
| pyrimidin-2-yl]piperidine-3,5-diol | | | | |
| 3,3-difluoro-1-[5-[[2-methyl-6- | A | A′ | A | C′ |
| (trifluoromethyl)phenyl]methoxy] | | | | |
| pyrimidin-2-yl]piperidin-4-ol | | | | |
| 1-[5-[[2-chloro-6- | A | A′ | A | A′ |
| (trifluoromethyl)phenyl]methoxy] | | | | |
| pyrimidin-2-yl]piperidin-4-ol | | | | |
| [1-[5-[[2-methyl-6- | B | A′ | A | A′ |
| (trifluoromethyl)phenyl]methoxy]-2- | | | | |
| pyridyl]imidazol-4-yl]methanamine | | | | |
| [1-[4-[[2-chloro-6- | B | A′ | B | A′ |
| (trifluoromethoxy)phenyl]methoxy] | | | | |
| phenyl]-1,2,4-triazol-3-yl]methanol | | | | |
| [1-[4-[[2-chloro-6- | B | B′ | B | A′ |
| (difluoromethoxy)phenyl]methoxy] | | | | |
| phenyl]-1,2,4-triazol-3-yl]methanol | | | | |
| 3-[4-[[2-chloro-6- | B | B′ | A | A′ |
| (trifluoromethyl)phenyl]methoxy] | | | | |
| phenyl]-1,3-diazaspiro[4.4]nonane-2,4- | | | | |
| dione | | | | |
| 3-[4-[[2-chloro-6- | B | A′ | A | A′ |
| (trifluoromethyl)phenyl]methoxy] | | | | |
| phenyl]-5,5-dimethyl-imidazolidine- | | | | |
| 2,4-dione | | | | |
| 3-[4-[[2-chloro-6- | B | B′ | A | A′ |
| (trifluoromethyl)phenyl]methoxy] | | | | |
| phenyl]-5-isopropyl-imidazolidine-2,4- | | | | |
| dione | | | | |
| 4-[5-[[2-chloro-6- | A | A′ | A | A′ |
| (trifluoromethyl)phenyl]methoxy] | | | | |
| pyrimidin-2-yl]piperazine-1- | | | | |
| carboxamide | | | | |
| (6R)-4-[5-[[2-chloro-6- | B | A′ | B | A′ |
| (trifluoromethyl)phenyl]methoxy] | | | | |
| pyrimidin-2-yl]-6- | | | | |
| (hydroxymethyl)piperazin-2-one | | | | |
| [1-[5-[(2,6- | B | B′ | B | A′ |
| dimethylphenyl)methoxy]-2- | | | | |
| pyridyl]pyrazol-3-yl]methanamine | | | | |
| [1-[5-[[2-(difluoromethoxy)-6- | B | C′ | B | A′ |
| methyl-phenyl]methoxy]pyrimidin- | | | | |
| 2-yl]pyrazol-3-yl]methanol | | | | |
| 1-[5-[[2-chloro-6- | B | A′ | B | A′ |
| (trifluoromethyl)phenyl]methoxy] | | | | |
| pyrimidin-2-yl]imidazolidine-2,4-dione | | | | |
| 4-[5-[[2-chloro-6- | B | A′ | B | A′ |
| (trifluoromethyl)phenyl]methoxy] | | | | |
| pyrimidin-2-yl]-6- | | | | |
| (hydroxymethyl)piperazin-2-one | | | | |
| [3-amino-1-[5-[[2-chloro-6- | B | B′ | C | A′ |
| (trifluoromethyl)phenyl]methoxy] | | | | |
| pyrimidin-2-yl]pyrrolidin-3- | | | | |
| yl]methanol | | | | |
| 6-[4-[[2-chloro-6- | B | A′ | A | A′ |
| (trifluoromethyl)phenyl]methoxy] | | | | |
| phenyl]-4,6-diazaspiro[2.4]heptane- | | | | |
| 5,7-dione | | | | |
| [1-[4-[[2-(difluoromethoxy)-6- | B | B′ | B | B′ |
| methyl-phenyl]methoxy]phenyl]- | | | | |
| 1,2,4-triazol-3-yl]methanol | | | | |
| 4-[5-[[2-chloro-6- | A | A′ | A | A′ |
| (trifluoromethyl)phenyl]methoxy] | | | | |
| pyrimidin-2-yl]piperidine-1- | | | | |
| carboxamide | | | | |
| 7-[5-[[2-chloro-6- | B | B′ | B | B′ |
| (trifluoromethyl)phenyl]methoxy] | | | | |
| pyrimidin-2-yl]-2,7- | | | | |
| diazaspiro[4.5]decane | | | | |
| (5S)-3-[5-[[2-chloro-6- | B | A′ | B | A′ |
| (trifluoromethyl)phenyl]methoxy]-2- | | | | |
| pyridyl]-5- | | | | |
| (hydroxymethyl)oxazolidin-2-one | | | | |
| (5R)-3-[5-[[2-chloro-6- | B | A′ | B | A′ |
| (trifluoromethyl)phenyl]methoxy]-2- | | | | |
| pyridyl]-5- | | | | |
| (hydroxymethyl)oxazolidin-2-one | | | | |
| 3-[5-[[2-chloro-6- | C | — | B | B′ |
| (trifluoromethyl)phenyl]methoxy]-2- | | | | |
| pyridyl]imidazolidine-2,4-dione | | | | |
| 6-[5-[[2-chloro-6- | B | B′ | B | A′ |
| (trifluoromethyl)phenyl]methoxy] | | | | |
| pyrimidin-2-yl]-2,6- | | | | |
| diazaspiro[3.3]heptane-2- | | | | |
| carboxamide | | | | |
| 1-[5-[[2-chloro-6- | C | — | C | — |
| (trifluoromethyl)phenyl]methoxy] | | | | |
| pyrimidin-2-yl]-1,2,4-triazole-3- | | | | |
| carboxylic acid | | | | |
| 1-[5-[[2-chloro-6- | B | B′ | B | A′ |
| (trifluoromethyl)phenyl]methoxy] | | | | |
| pyrimidin-2-yl]pyrrolidine-3- | | | | |
| carboxylic acid | | | | |
| 4-[5-[[2-chloro-6- | B | A′ | B | A′ |
| (trifluoromethyl)phenyl]methoxy] | | | | |
| pyrimidin-2-yl]morpholine-2- | | | | |
| carboxamide | | | | |
| 2-[5-[[2-chloro-6- | B | A′ | C | A′ |
| (trifluoromethyl)phenyl]methoxy] | | | | |
| pyrimidin-2-yl]-2,9- | | | | |
| diazaspiro[4.5]decane | | | | |
| 4-[5-[[2-chloro-6- | B | A′ | B | A′ |
| (trifluoromethyl)phenyl]methoxy]-2- | | | | |
| pyridyl]-6- | | | | |
| (hydroxymethyl)morpholin-3-one | | | | |
| 4-[5-[[2-chloro-6- | B | A′ | A | A′ |
| (trifluoromethyl)phenyl]methoxy] | | | | |
| pyrimidin-2-yl]piperazin-2-one | | | | |
| 6-[(2,6- | C | — | C | — |
| dichlorophenyl)methylamino]-4H- | | | | |
| isoquinoline-1,3-dione | | | | |
| [4-[5-[[2-methyl-6- | B | A′ | B | A′ |
| (trifluoromethyl)phenyl]methoxy] | | | | |
| pyrimidin-2-yl]-1,1-dioxo-1,4- | | | | |
| thiazinan-2-yl]methanol | | | | |
| 1-[5-[[2-chloro-6- | A | — | B | B′ |
| (trifluoromethyl)phenyl]methoxy]-2- | | | | |
| pyridyl]-1,2,4-triazole-3-carboxylic | | | | |
| acid | | | | |
| 4-[5-[[2-chloro-6- | A | — | B | C′ |
| (trifluoromethyl)phenyl]methoxy] | | | | |
| pyrimidin-2-yl]morpholine-2- | | | | |
| carboxylic acid | | | | |
| 1-[5-[[2-chloro-6- | A | — | B | C′ |
| (trifluoromethyl)phenyl]methoxy] | | | | |
| pyrimidin-2-yl]piperidine-3-carboxylic | | | | |
| acid | | | | |
| 1-[5-[[2-chloro-6- | B | A′ | B | A; |
| (trifluoromethyl)phenyl]methoxy] | | | | |
| pyrimidin-2-yl]piperidine-4-carboxylic | | | | |
| acid | | | | |
| 3-[5-[[2-chloro-6- | B | A′ | B | A′ |
| (trifluoromethyl)phenyl]methoxy] | | | | |
| pyrimidin-2-yl]-5- | | | | |
| (hydroxymethyl)oxazolidin-2-one | | | | |
| 5-{[2-chloro-6- | B | C′ | — | — |
| (trifluoromethyl)phenyl]methoxy}- | | | | |
| 2-[3-(trifluoromethyl)- | | | | |
| 5H,6H,7H,8H-[1,2,4]triazolo[4,3- | | | | |
| a]pyrazin-7-yl]pyrimidine | | | | |
| 6-{[(2,6- | C | — | C | — |
| dichlorophenyl)methyl]amino}- | | | | |
| 1,2,3,4-tetrahydroisoquinoline-1,3- | | | | |
| dione | | | | |
| 2-(hydroxymethyl)-4-(5-{[2-methyl- | B | — | B | C′ |
| 6- | | | | |
| (trifluoromethyl)phenyl]methoxy} | | | | |
| pyrimidin-2-yl)-1λ⁶-thiomorpholine- | | | | |
| 1,1-dione | | | | |
| 1-(5-{[2-chloro-6- | C | — | B | C′ |
| (trifluoromethyl)phenyl]methoxy] | | | | |
| pyridin-2-yl)-1H-1,2,4-triazole-3- | | | | |
| carboxylic acid | | | | |
| 4-(5-{[2-chloro-6- | C | — | B | — |
| (trifluoromethyl)phenyl]methoxy} | | | | |
| pyrimidin-2-yl)morpholine-2- | | | | |
| carboxylic acid | | | | |
| 1-(5-{[2-chloro-6- | C | — | B | — |
| (trifluoromethyl)phenyl]methoxy] | | | | |
| pyrimidin-2-yl)piperidine-3-carboxylic | | | | |
| acid | | | | |
| 1-(5-{[2-chloro-6- | B | C′ | B | C′ |
| (trifluoromethyl)phenyl]methoxy} | | | | |
| pyrimidin-2-yl)piperidine-4-carboxylic | | | | |
| acid | | | | |
| [5-(5-{[2-chloro-6- | A | C′ | A | C′ |
| (trifluoromethyl)phenyl]methoxy} | | | | |
| pyridin-2-yl)-1,3,4-oxadiazol-2- | | | | |
| yl]methanol | | | | |
| 4-(5-{[2-chloro-6- | C | C′ | B | C′ |
| (trifluoromethyl)phenyl]methoxy] | | | | |
| pyridin-2-yl)morpholine-2-carboxylic | | | | |
| acid | | | | |
| rac-1-[(2R)-2-(aminomethyl)-4-(5- | B | C′ | B | C′ |
| ([2-methyl-6- | | | | |
| (trifluoromethyl)phenyl]methoxy} | | | | |
| pyrimidin-2-yl)piperazin-1-yl]ethan-1- | | | | |
| one | | | | |
| [1-(4-{[2-chloro-6- | A | C′ | A | C′ |
| (trifluoromethyl)phenyl](²H₂) | | | | |
| methoxy}phenyl)-1H-1,2,4-triazol-3- | | | | |
| yl](²H₂)methanol | | | | |
| (4R)-1-(5-{[2-chloro-6- | B | C′ | B | C′ |
| (trifluoromethyl)phenyl]methoxy} | | | | |
| pyrimidin-2-yl)-4- | | | | |
| (hydroxymethyl)imidazolidin-2-one | | | | |
| (5S)-3-(5-{[2-chloro-6- | C | C′ | B | C′ |
| (trifluoromethyl)phenyl]methoxy} | | | | |
| pyrimidin-2-yl)-5-(hydroxymethyl)- | | | | |
| 1,3-oxazolidin-2-one | | | | |
| 3-{5-[(2,6- | C | — | C | — |
| dichlorophenyl)difluoromethoxy] | | | | |
| pyrimidin-2-yl}-5-(hydroxymethyl)-1,3- | | | | |
| oxazolidin-2-one | | | | |
| 4-(5-{[2-chloro-6- | B | C′ | A | C′ |
| (trifluoromethyl)phenyl]methoxy] | | | | |
| pyrimidin-2-yl)-6-(hydroxymethyl)-1- | | | | |
| methylpiperazin-2-one | | | | |
| 4-(5-{[2-chloro-6- | B | C′ | B | C′ |
| (trifluoromethyl)phenyl]methoxy} | | | | |
| pyrimidin-2-yl)-6-oxopiperazine-2- | | | | |
| carboxamide | | | | |
| (1-{5-[(2,6- | B | C′ | B | C′ |
| dichlorophenyl)methoxy]pyrimidin- | | | | |
| 2-yl}-1H-1,2,4-triazol-3-yl)methanol | | | | |
| (4S)-1-(5-([2-ethyl-6- | A | C′ | A | C′ |
| (trifluoromethyl)phenyl]methoxy] | | | | |
| pyridin-2-yl)-4- | | | | |
| (hydroxymethyl)imidazolidin-2-one | | | | |
| (1-(5-[(2,6- | B | C′ | — | — |
| dichlorophenyl)(²H₂)methoxy] | | | | |
| pyrimidin-2-yl]-1H-pyrazol-3- | | | | |
| yl)(²H₂)methanol | | | | |
| 1-{5-[(2,6- | B | C′ | B | C′ |
| dichlorophenyl)methoxy]pyrimidin- | | | | |
| 2-yl}imidazolidine-2,4-dione | | | | |
| [1-(5-{[2-chloro-6- | A | C′ | A | C′ |
| (trifluoromethyl)phenyl]methoxy} | | | | |
| pyrimidin-2-yl)-1H-imidazol-4- | | | | |
| yl]methanol | | | | |
| (4S)-1-(5-{[2-chloro-6- | A | C′ | — | — |
| (trifluoromethyl)phenyl]methoxy} | | | | |
| pyridin-2-yl)-4- | | | | |
| (hydroxymethyl)imidazolidin-2-one | | | | |
| (4S)-1-(5-{[2,6- | C | C′ | — | — |
| bis(trifluoromethyl)phenyl]methoxy} | | | | |
| pyrimidin-2-yl)-4- | | | | |
| (hydroxymethyl)imidazolidin-2-one | | | | |
| 1-[(2S)-1-methyl-4-(5-{[2-methyl-6- | C | C′ | — | — |
| (trifluoromethyl)phenyl]methoxy} | | | | |
| pyrimidin-2-yl)piperazin-2- | | | | |
| yl]methanamine | | | | |
| [1-(5-{[2-chloro-6- | B | C′ | — | — |
| (trifluoromethyl)phenyl](²H₂)methoxy} | | | | |
| pyrimidin-2-yl)-1H-1,2,4-triazol- | | | | |
| 3-yl](²H₂)methanol | | | | |
| [2-(5-{[2-methyl-6- | A | C′ | — | — |
| (trifluoromethyl)phenyl]methoxy} | | | | |
| pyrimidin-2-yl)-2H-1,2,3-triazol-4- | | | | |
| yl]methanol | | | | |
| (4S)-4-(hydroxymethyl)-1-(5-{[2- | B | C′ | — | — |
| methyl-6- | | | | |
| (trifluoromethyl)phenyl]methoxy} | | | | |
| pyrimidin-2-yl)imidazolidin-2-one | | | | |
| (4S)-1-(5-([2-ethyl-6- | B | C′ | — | — |
| (trifluoromethyl)phenyl]methoxy} | | | | |
| pyrimidin-2-yl)-4- | | | | |
| (hydroxymethyl)imidazolidin-2-one | | | | |
| 1-{5-[(2,6- | A | C′ | — | — |
| dichlorophenyl)methoxy]pyridin-2- | | | | |
| yl}imidazolidine-2,4-dione | | | | |
| (4S)-1-(5-{[2,6- | A | C′ | — | — |
| bis(trifluoromethyl)phenyl]methoxy} | | | | |
| pyridin-2-yl)-4- | | | | |
| (hydroxymethyl)imidazolidin-2-one | | | | |
| [1-(5-{[2-chloro-6- | A | C′ | — | — |
| (trifluoromethyl)phenyl]methoxy} | | | | |
| pyrimidin-2-yl)-1H-pyrazol-3- | | | | |
| yl]methanol | | | | |
| 1-(5-{[2-methyl-6- | A | C′ | — | — |
| (trifluoromethyl)phenyl]methoxy} | | | | |
| pyrimidin-2-yl)-1H-pyrazole-3- | | | | |
| carboxamide | | | | |
| 1-{5-[(2,6- | B | C′ | — | — |
| dichlorophenyl)methoxy]pyrazin-2- | | | | |
| yl}imidazolidine-2,4-dione | | | | |
| (1-{5-[(2,6- | A | C′ | — | — |
| dichlorophenyl)methoxy]pyridin-2- | | | | |
| yl}-1H-1,2,4-triazol-3-yl)methanol | | | | |
| 1-(5-{[2-chloro-6- | B | C′ | — | — |
| (trifluoromethyl)phenyl]methoxy} | | | | |
| pyrazin-2-yl)imidazolidine-2,4-dione | | | | |
| (1-{5-[(2,6- | B | C′ | — | — |
| dichlorophenyl)methoxy]pyrazin-2- | | | | |
| yl]-1H-pyrazol-3-yl)methanol | | | | |
| 3-{5-[(2,6-dichloro-3- | B | C′ | — | — |
| fluorophenyl)methoxy]pyridin-2- | | | | |
| yl}-5-(hydroxymethyl)-1,3- | | | | |
| oxazolidin-2-one | | | | |
| 1-{5-[(2,6-dichloro-3- | A | C′ | — | — |
| fluorophenyl)methoxy]pyridin-2- | | | | |
| yl}imidazolidine-2,4-dione | | | | |
| 1-(5-{[2-chloro-3-fluoro-6- | A | C′ | — | — |
| (trifluoromethyl)phenyl]methoxy} | | | | |
| pyridin-2-yl)imidazolidine-2,4-dione | | | | |
| [1-(5-{[2-chloro-3-fluoro-6- | A | C′ | — | — |
| (trifluoromethyl)phenyl]methoxy} | | | | |
| pyridin-2-yl)-1H-1,2,4-triazol-3- | | | | |
| yl]methanol | | | | |
| (1-{5-[(2,6-dichloro-4- | B | C′ | — | — |
| fluorophenyl)methoxy]pyrimidin-2- | | | | |
| yl}-1H-imidazol-4-yl)methanol | | | | |
| 3-{5-[(2,6-dichloro-3- | B | C′ | — | — |
| fluorophenyl)methoxy]pyrimidin-2- | | | | |
| yl}-5-(hydroxymethyl)-1,3- | | | | |
| oxazolidin-2-one | | | | |
| (1-{5-[(2,6-dichloro-3- | B | C′ | — | — |
| fluorophenyl)methoxy]pyrimidin-2- | | | | |
| yl}-1H-1,2,3-triazol-4-yl)methanol | | | | |
| (2-{5-[(2,6-dichloro-3- | A | C′ | — | — |
| fluorophenyl)methoxy]pyrimidin-2- | | | | |
| yl}-2H-1,2,3-triazol-4-yl)methanol | | | | |
| (1-{5-[(2,6-dichloro-3- | A | C′ | — | — |
| fluorophenyl)methoxy]pyrimidin-2- | | | | |
| yl]-1H-imidazol-4-yl)methanol | | | | |
| 1-{5-[(2,6-dichloro-4- | B | C′ | — | — |
| fluorophenyl)methoxy]pyridin-2- | | | | |
| yl}imidazolidine-2,4-dione | | | | |
| [1-(4-{[3-fluoro-2-methyl-6- | A | C′ | — | — |
| (trifluoromethyl)phenyl]methoxy} | | | | |
| phenyl)-1H-1,2,4-triazol-3-yl]methanol | | | | |
| 1-{6-[(2,6- | B | C′ | — | — |
| dichlorophenyl)methoxy]pyridazin- | | | | |
| 3-yl}imidazolidine-2,4-dione | | | | |
| [1-(5-{[2-chloro-3-fluoro-6- | A | C′ | — | — |
| (trifluoromethyl)phenyl]methoxy} | | | | |
| pyrimidin-2-yl)-1H-imidazol-4- | | | | |
| yl]methanol | | | | |
| 1-[(2S)-2-(aminomethyl)-4-(5-{[2- | B | B′ | — | — |
| chloro-6- | | | | |
| (trifluoromethyl)phenyl]methoxy} | | | | |
| pyrimidin-2-yl)piperazin-1-yl]ethan-1- | | | | |
| one | | | | |
| 1-(5-{[2-chloro-6- | A | A′ | — | — |
| (trifluoromethyl)phenyl]methoxy} | | | | |
| pyrimidin-2-yl)azetidin-3-one | | | | |
| (1-{5-[2-(2,6- | B | C′ | — | — |
| dichlorophenyl)ethyl]pyrimidin-2- | | | | |
| yl}-1H-1,2,4-triazol-3-yl)methanol | | | | |
| [1-(5-{[2-chloro-3-fluoro-6- | B | A′ | — | — |
| (trifluoromethyl)phenyl]methoxy} | | | | |
| pyrimidin-2-yl)-1H-1,2,4-triazol-3- | | | | |
| yl]methanol | | | | |
| [1-(5-{[6-chloro-3-fluoro-2- | A | A′ | — | — |
| (trifluoromethyl)phenyl]methoxy] | | | | |
| pyrimidin-2-yl)-1H-1,2,4-triazol-3- | | | | |
| yl]methanol | | | | |
| 1-(5-{[6-chloro-3-fluoro-2- | A | A′ | — | — |
| (trifluoromethyl)phenyl]methoxy} | | | | |
| pyridin-2-yl)imidazolidine-2,4-dione | | | | |
| (1-{5-[(2,6- | B | A′ | — | — |
| dichlorophenyl)methoxy]pyrimidin- | | | | |
| 2-yl}-1H-imidazol-5-yl)methanol | | | | |
| 1-(5-{[2,6- | A | A′ | — | — |
| bis(trifluoromethyl)phenyl]methoxy} | | | | |
| pyridin-2-yl)imidazolidine-2,4- | | | | |
| dione | | | | |
| [1-(5-{[2,6- | A | A′ | — | — |
| bis(trifluoromethyl)phenyl]methoxy} | | | | |
| pyrimidin-2-yl)-1H-pyrazol-3- | | | | |
| yl]methanol | | | | |
| 1-(5-{[2-chloro-6- | A | A′ | — | — |
| (trifluoromethyl)phenyl]methoxy} | | | | |
| pyrimidin-2-yl)-3-hydroxyazetidine-3- | | | | |
| carbonitrile | | | | |
| [1-(4-{[3-fluoro-6-methyl-2- | A | A′ | — | — |
| (trifluoromethyl)phenyl]methoxy} | | | | |
| phenyl)-1H-1,2,4-triazol-3-yl]methanol | | | | |
| [1-(5-{[2,6- | A | A′ | — | — |
| bis(trifluoromethyl)phenyl]methoxy} | | | | |
| pyrimidin-2-yl)-1H-imidazol-4- | | | | |
| yl]methanol | | | | |
| 1-(5-{[2,6- | A | A′ | — | — |
| bis(trifluoromethyl)phenyl]methoxy} | | | | |
| pyrimidin-2-yl)-1H-pyrazole-3- | | | | |
| carboxamide | | | | |

A: less than 1.0 μM;
B: between 1.0 and 10.0 μM,
C: greater than 10.0 μM;
A′ = ≥10X,
B′ = ≥5X to <10X,
C′ = <5X

### Example B—Measuring functional CFTR channel activity using TECC-24 Electrophysiology Platform

16HBE human bronchial epithelial cells were seeded onto Transwell 24-well filter inserts coated with human collagen type IV. Cells were seeded 7 days prior to functional testing at a density of 1.7×10⁵ cells onto the “apical” chamber filter of the Transwell insert. Cells were grown as submerged cultures (200 μL apical/700 μL basolateral) in Minimal Essential Medium (MEM) containing 10% qualified Fetal Bovine Serum and 1% Penicillin/Streptomycin and incubated at 37° C. and 5% CO₂. Growth media on cells was replenished with fresh media every 1-3 days prior to test agent treatment day. After 7 days in culture, the CFTR-mediated chloride (Cl⁻) equivalent current (Ieq) of the 16HBE cells was recorded, which may typically form electrically tight epithelia with a transepithelial resistance (Rt) of 2000-6000 Ω·cm2 as described below. Forty-eight hours prior to electrophysiological measurements, fresh growth media containing the test agents was applied to both chambers separating filter bound epithelial monolayer.

On the day of electrophysiology studies, electrodes of a “Multi-(24-channel) transepithelial current clamp amplifier” (MTECC-24 (TECC-24 for short), EP Devices, Belgium) were soaked in an assay buffer (see below for composition) for at least 2 hours using 24-well Transwell plates without inserts. Cell culture media was replaced in the Transwell assay plates with assay buffer (see below) and the plates were mounted onto a custom-built automated robotic assay platform (Yamaha RCX240S controller) with heated plate holders set at 37° C. for 60 min. After equilibration, transepithelial potential difference (PD) and resistance (Rt) was monitoredat ˜5 min intervals using the TECC-24 transepithelial current clamp. Electrode potential differences were monitored for each pair of Ag/AgCl voltage electrodes by taking voltage measurements from a control (“blank”) plate with matching buffer solutions and 16HBE cells that were not treated with any test agent. PD values for each electrode pair were subtracted from the blank plate from that of the test plates at each time point. Ieq was calculated from the corrected PD values and Rt using Ohm's law. Ieq values were slope corrected to account for measurement drift over time using the equation: Slope corrected value=calculated Ieq−((time x slope)+y-intercept) with the slope and y-intercept calculated from the data points captured during the initial baseline period and following addition of the CFTR inhibitor.

The equivalent current (Ieq) traces associated with CFTR channel activation were plotted as baseline subtracted mean±standard deviation from 3-4 separate wells on the 24-well Transwell (n=3-4). There are 4 recording periods, with the first set of data points recorded baseline Ieq currents prior to sequential activation of available CFTR channels with forskolin (10 μM) and then in combination with potentiator VX-770/ivacaftor (1 μM). Recorded the last set of data points in the presence of CFTR inhibitor CFTRinh-172 (20 μM) to confirm that currents are mediated via activation of CFTR channels. The test agents and control/reference compounds were pre-diluted to 10-fold concentrations in assay buffer and added manually using a multi-channel pipette to both the basolateral and apical side of the membrane (test plates only). Employed CFTR-mediated changes in Ieq, (i.e. forskolin/VX-770 activated currents) to measure functional CFTR surface expression or treatment-related functional rescue of mutant CFTR.

The magnitude of test agent mediated enhancement of functional CFTR currents was calculated by comparing forskolin/VX-770 stimulated Ieq relative to baseline/CFTRinh-172 Ieq following 48 hour pretreatment with test agent(s) and normalizing to the forskolin/VX-770 stimulated Ieq determined following 48 hour pretreatment with a maximally effective concentration a reference control aminoglycoside agent (typically 110 μM G418). In other treatments, the compounds of the current disclosure were administered with with both a sub maximally effective concentration of aminoglycoside (i.e. 10 μM G418) and a cereblon E3 ligase modulator that promotes degradation of proteins including GSPT1 (Drug Discov Today Technol 2019; 31:29-34. doi: 10.1016/j.ddtec.2019.02.004, J Med Chem. 2021 Feb. 25;64 (4): 1835-1843. doi: 10.1021/acs.jmedchem.0c01489.) (Concentrations ranging from 1 nM to 10 μM).

Assay buffer: The CFTR-mediated transepithelial currents was recorded on TECC-24 using a C₁-concentration gradient to provide a driving force for chloride ions. An asymmetrical chloride gradient was established using the following assay buffer (applied at 200 μL apical/750 μL basolateral sides epithelia). The basolateral solution contained (mM): 137 NaCl, 4 KCl, 1.8 CaCl₂), 1 MgCl₂, 10 HEPES and D-Glucose, adjusted to pH 7.4 with NaOH. The apical solution was matched to the basolateral except for (mM): 137 Na-gluconate replaced 137 NaCl.

Amplification of CFTR R1162X readthrough using combinations of readthrough modulator compound, aminoglycoside and the eRF3 modulator CC-90009 is provided in Table 2, whereas amplification of G542X readthrough using combinations of readthrough modulator compound, aminoglycoside and the eRF3 modulator CC-90009 is provided in Table 3. The readthrough modulators increased potency of aminoglycoside enhancement of CFTR PTC mutant readthrough so that maximal readthrough can be achieved at lower aminoglycosides concentrations. Enhancement was even more significant when eRF3 modulators were included.

TABLE 2
Amplification of CFTR R1162X readthrough by combinations of readthrough
modulators with Aminoglycoside and Cereblon E3 ligase eRF3 modulators

| | | eRF3 | | | Currents |
|---|---|---|---|---|---|
| | | modu- | | | (% of 110 |
| | RTM | lator | | | μM G418 |
| Read-through modulator | Conc. | conc. | | aminoglycoside | (Max |
| compound (RTM) | (uM) | (uM) | aminoglycoside | conc. (uM) | effect)) |
| 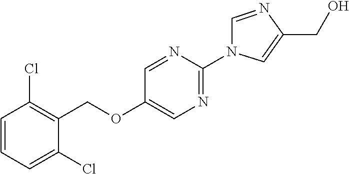 [SMILES: OCc1cn(-c2ncc(OCc3c(Cl)cccc3Cl)cn2)cn1] | 0.5 | — | G418 | 10 | 64 |
| — | — | 1 | G418 | 10 | 110 |
| 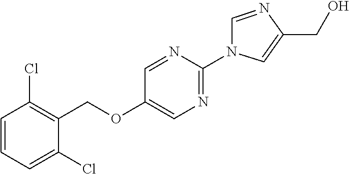 [SMILES: OCc1cn(-c2ncc(OCc3c(Cl)cccc3Cl)cn2)cn1] | 0.5 | 1 | G418 | 10 | 472 |
| 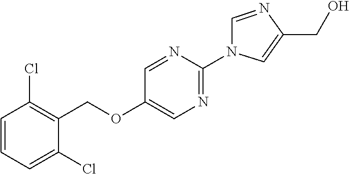 [SMILES: OCc1cn(-c2ncc(OCc3c(Cl)cccc3Cl)cn2)cn1] | 0.5 | — | ELX-02 | 100 | 51 |
| — | — | 1 | ELX-02 | 100 | 198 |
| 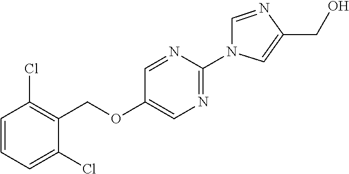 [SMILES: OCc1cn(-c2ncc(OCc3c(Cl)cccc3Cl)cn2)cn1] | 0.5 | 1 | ELX-02 | 100 | 473 |
| 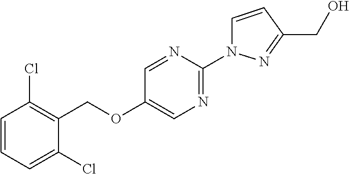 [SMILES: OCc1ccn(-c2ncc(OCc3c(Cl)cccc3Cl)cn2)n1] | 1 | — | ELX-02 | 100 | 102 |
| 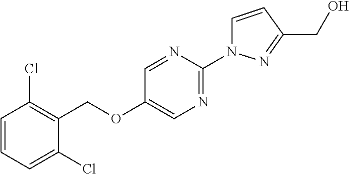 [SMILES: OCc1ccn(-c2ncc(OCc3c(Cl)cccc3Cl)cn2)n1] | — | 1 | ELX-02 | 100 | 63 |
| 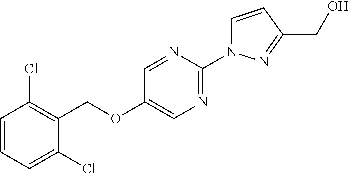 [SMILES: OCc1ccn(-c2ncc(OCc3c(Cl)cccc3Cl)cn2)n1] | 1 | 1 | ELX-02 | 100 | 300 |
| 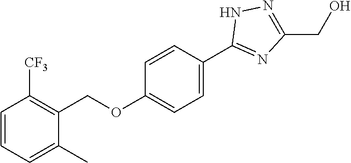 [SMILES: Cc1cccc(C(F)(F)F)c1COc1ccc(-c2nc(CO)n[nH]2)cc1] | 1 | — | ELX-02 | 100 | 106 |
| 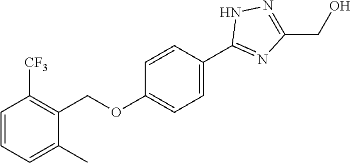 [SMILES: Cc1cccc(C(F)(F)F)c1COc1ccc(-c2nc(CO)n[nH]2)cc1] | — | 1 | ELX-02 | 100 | 63 |
| 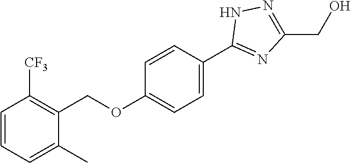 [SMILES: Cc1cccc(C(F)(F)F)c1COc1ccc(-c2nc(CO)n[nH]2)cc1] | 1 | 1 | ELX-02 | 100 | 269 |
| 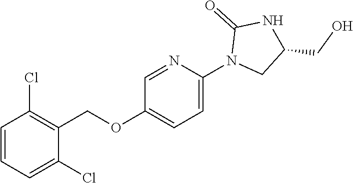 [SMILES: O=C1N[C@H](CO)CN1c1ccc(OCc2c(Cl)cccc2Cl)cn1] | 1 | — | ELX-02 | 100 | 70 |
| 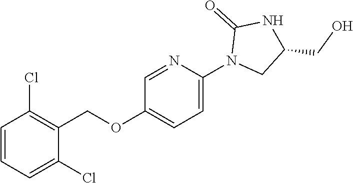 [SMILES: O=C1N[C@H](CO)CN1c1ccc(OCc2c(Cl)cccc2Cl)cn1] | — | 1 | ELX-02 | 100 | 63 |
| 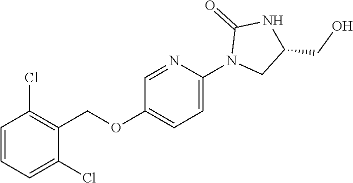 [SMILES: O=C1N[C@H](CO)CN1c1ccc(OCc2c(Cl)cccc2Cl)cn1] | 1 | 1 | ELX-02 | 100 | 266 |
| 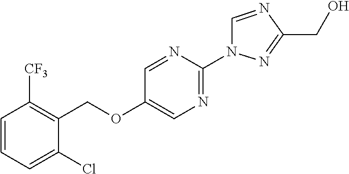 [SMILES: OCc1ncn(-c2ncc(OCc3c(Cl)cccc3C(F)(F)F)cn2)n1] | 3 | — | ELX-02 | 100 | 102 |
| 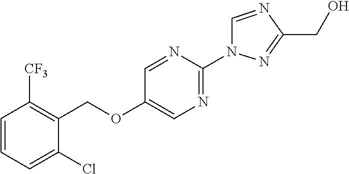 [SMILES: OCc1ncn(-c2ncc(OCc3c(Cl)cccc3C(F)(F)F)cn2)n1] | — | 1 | ELX-02 | 100 | 77 |
| 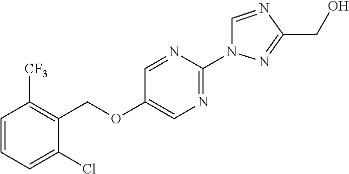 [SMILES: OCc1ncn(-c2ncc(OCc3c(Cl)cccc3C(F)(F)F)cn2)n1] | 3 | 1 | ELX-02 | 100 | 288 |
| 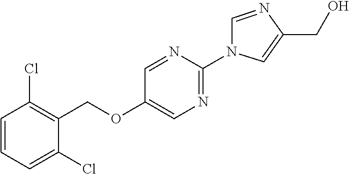 [SMILES: OCc1cn(-c2ncc(OCc3c(Cl)cccc3Cl)cn2)cn1] | 0.5 | — | Paramomycin | 2000 | 7 |
| — | — | 1 | Paramomycin | 2000 | 55 |
| 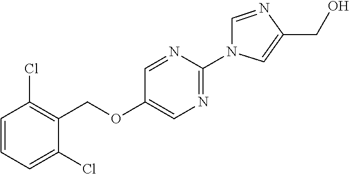 [SMILES: OCc1cn(-c2ncc(OCc3c(Cl)cccc3Cl)cn2)cn1] | 0.5 | 1 | Paramomycin | 2000 | 183 |
| 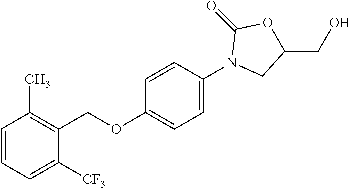 [SMILES: Cc1cccc(C(F)(F)F)c1COc1ccc(N2CC(CO)OC2=O)cc1] | 1 | — | G418 | 10 | 76 |
| — | — | 1 | G418 | 10 | 95 |
| 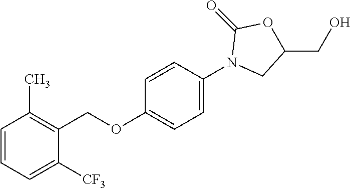 [SMILES: Cc1cccc(C(F)(F)F)c1COc1ccc(N2CC(CO)OC2=O)cc1] | 1 | 1 | G418 | 10 | 423 |
| 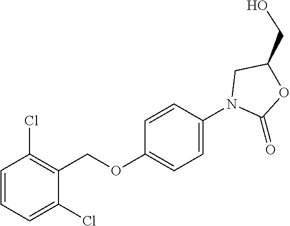 [SMILES: O=C1O[C@H](CO)CN1c1ccc(OCc2c(Cl)cccc2Cl)cc1] | 5 | — | G418 | 10 | 54 |
| — | — | 10 | G418 | 10 | 130 |
| 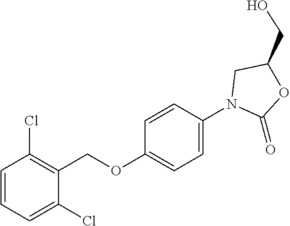 [SMILES: O=C1O[C@H](CO)CN1c1ccc(OCc2c(Cl)cccc2Cl)cc1] | 5 | 10 | G418 | 10 | 314 |
| 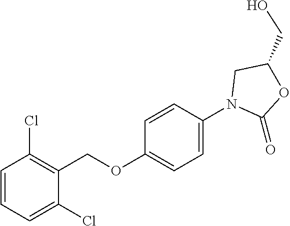 [SMILES: O=C1O[C@@H](CO)CN1c1ccc(OCc2c(Cl)cccc2Cl)cc1] | 5 | — | G418 | 10 | 81 |
| — | — | 10 | G418 | 10 | 139 |
| 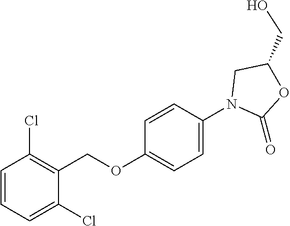 [SMILES: O=C1O[C@@H](CO)CN1c1ccc(OCc2c(Cl)cccc2Cl)cc1] | 5 | 10 | G418 | 10 | 320 |

TABLE 3
Amplification of CFTR G542X readthrough by combinations of readthrough
modulators with Aminoglycoside and Cereblon E3 ligase eRF3 modulators

| | | eRF3 | | | Currents |
|---|---|---|---|---|---|
| | | modulator | | | (% of 110 |
| | RTM | conc. | | | μM G418 |
| Read-through modulator | Conc. | (uM) | | aminoglycoside | (Max |
| compound (RTM) | (uM) | | aminoglycoside | conc. (uM) | effect)) |
| 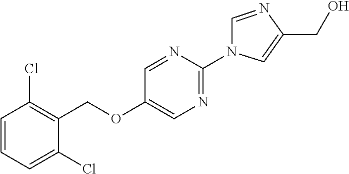 [SMILES: OCc1cn(-c2ncc(OCc3c(Cl)cccc3Cl)cn2)cn1] | 0.5 | — | G418 | 10 | 78 |
| — | — | 1 | G418 | 10 | 130 |
| 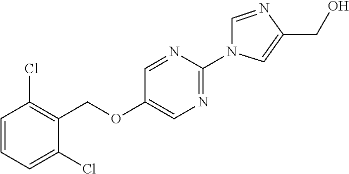 [SMILES: OCc1cn(-c2ncc(OCc3c(Cl)cccc3Cl)cn2)cn1] | 0.5 | 1 | G418 | 10 | 429 |
| 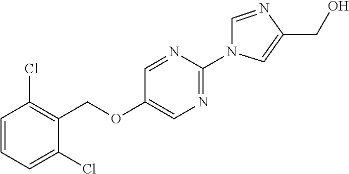 [SMILES: OCc1cn(-c2ncc(OCc3c(Cl)cccc3Cl)cn2)cn1] | 0.5 | — | ELX-02 | 100 | 50 |
| — | — | 1 | ELX-02 | 100 | 191 |
| 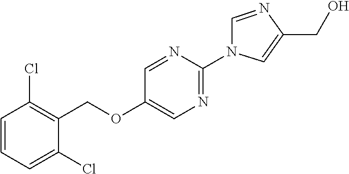 [SMILES: OCc1cn(-c2ncc(OCc3c(Cl)cccc3Cl)cn2)cn1] | 0.5 | 1 | ELX-02 | 100 | 421 |
| 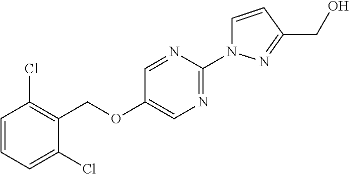 [SMILES: OCc1ccn(-c2ncc(OCc3c(Cl)cccc3Cl)cn2)n1] | 1 | — | ELX-02 | 100 | 94 |
| 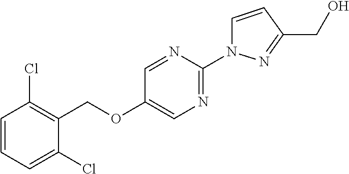 [SMILES: OCc1ccn(-c2ncc(OCc3c(Cl)cccc3Cl)cn2)n1] | — | 1 | ELX-02 | 100 | 83 |
| 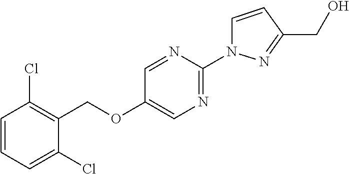 [SMILES: OCc1ccn(-c2ncc(OCc3c(Cl)cccc3Cl)cn2)n1] | 1 | 1 | ELX-02 | 100 | 278 |
| 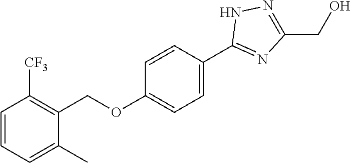 [SMILES: Cc1cccc(C(F)(F)F)c1COc1ccc(-c2nc(CO)n[nH]2)cc1] | 1 | — | ELX-02 | 100 | 109 |
| 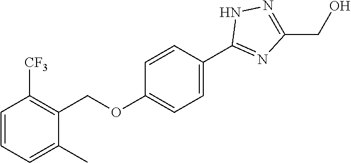 [SMILES: Cc1cccc(C(F)(F)F)c1COc1ccc(-c2nc(CO)n[nH]2)cc1] | — | 1 | ELX-02 | 100 | 83 |
| 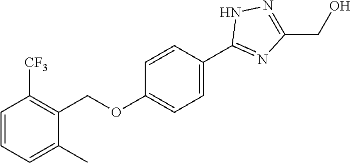 [SMILES: Cc1cccc(C(F)(F)F)c1COc1ccc(-c2nc(CO)n[nH]2)cc1] | 1 | 1 | ELX-02 | 100 | 263 |
| 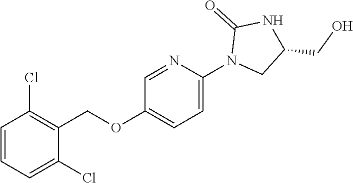 [SMILES: O=C1N[C@H](CO)CN1c1ccc(OCc2c(Cl)cccc2Cl)cn1] | 1 | -- | ELX-02 | 100 | 79 |
| 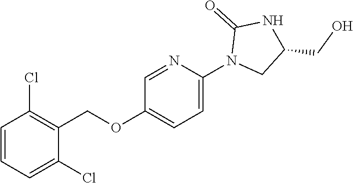 [SMILES: O=C1N[C@H](CO)CN1c1ccc(OCc2c(Cl)cccc2Cl)cn1] | — | 1 | ELX-02 | 100 | 83 |
| 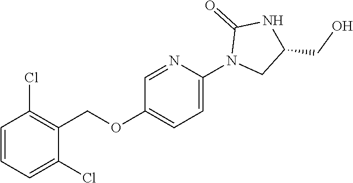 [SMILES: O=C1N[C@H](CO)CN1c1ccc(OCc2c(Cl)cccc2Cl)cn1] | 1 | 1 | ELX-02 | 100 | 293 |
| 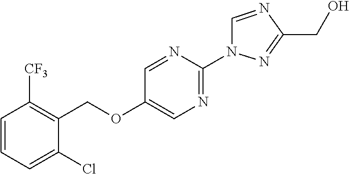 [SMILES: OCc1ncn(-c2ncc(OCc3c(Cl)cccc3C(F)(F)F)cn2)n1] | 3 | — | ELX-02 | 100 | 111 |
| 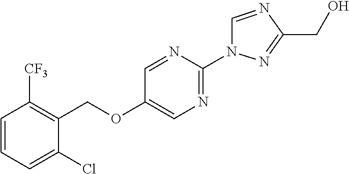 [SMILES: OCc1ncn(-c2ncc(OCc3c(Cl)cccc3C(F)(F)F)cn2)n1] | — | 1 | ELX-02 | 100 | 60 |
| 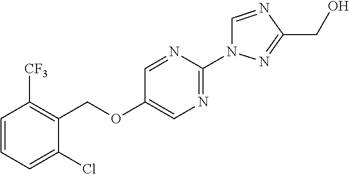 [SMILES: OCc1ncn(-c2ncc(OCc3c(Cl)cccc3C(F)(F)F)cn2)n1] | 3 | 1 | ELX-02 | 100 | 263 |
| 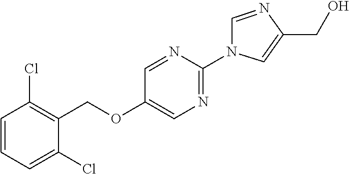 [SMILES: OCc1cn(-c2ncc(OCc3c(Cl)cccc3Cl)cn2)cn1] | 0.5 | -- | Paramomycin | 2000 | 5 |
| — | — | 1 | Paramomycin | 2000 | 65 |
| 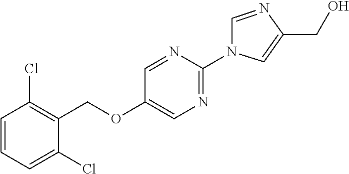 [SMILES: OCc1cn(-c2ncc(OCc3c(Cl)cccc3Cl)cn2)cn1] | 0.5 | 1 | Paramomycin | 2000 | 140 |
| 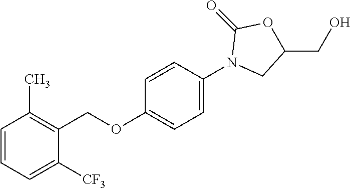 [SMILES: Cc1cccc(C(F)(F)F)c1COc1ccc(N2CC(CO)OC2=O)cc1] | 1 | — | G418 | 10 | 76 |
| — | — | 1 | G418 | 10 | 130 |
| 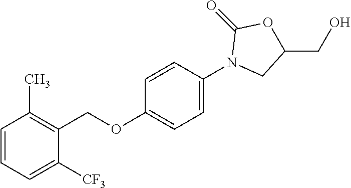 [SMILES: Cc1cccc(C(F)(F)F)c1COc1ccc(N2CC(CO)OC2=O)cc1] | 1 | 1 | G418 | 10 | 290 |
| 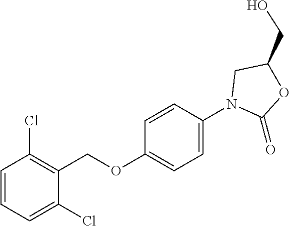 [SMILES: O=C1O[C@H](CO)CN1c1ccc(OCc2c(Cl)cccc2Cl)cc1] | 5 | — | G418 | 10 | 54 |
| — | — | 10 | G418 | 10 | 102 |
| 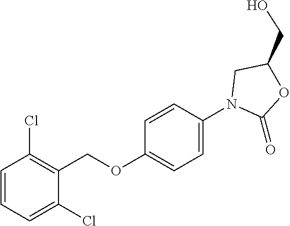 [SMILES: O=C1O[C@H](CO)CN1c1ccc(OCc2c(Cl)cccc2Cl)cc1] | 5 | 10 | G418 | 10 | 323 |

With respect to the use of substantially any plural and/or singular terms herein, those having skill in the art can translate from the plural to the singular and/or from the singular to plural as is appropriate to the context and/or application. The various singular/plural permutations can be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims can contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A compound having the structure of Formula (I) 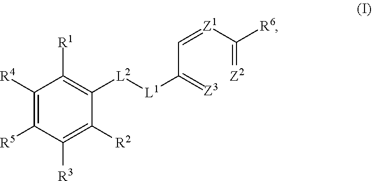
or a pharmaceutically acceptable salt thereof, wherein:
R¹, R², R³, R⁴, and R⁵ are each independently selected from the group consisting of: hydrogen, halo, —CN, —NO₂, —OH, C₁₋₆ alkyl, C₁₋₆ haloalkyl, C₁₋₆ alkoxy, C₁₋₆ haloalkoxy, and C₃₋₈ cycloalkyl;
L¹ is O, or CH₂;
L² is CH₂, CHF, or CF₂;
Z¹ is CH, N, or C—OCH₃;
Z² is CH or N;
Z³ is CH, N, or CF; and
R⁶ is selected from the group consisting of: 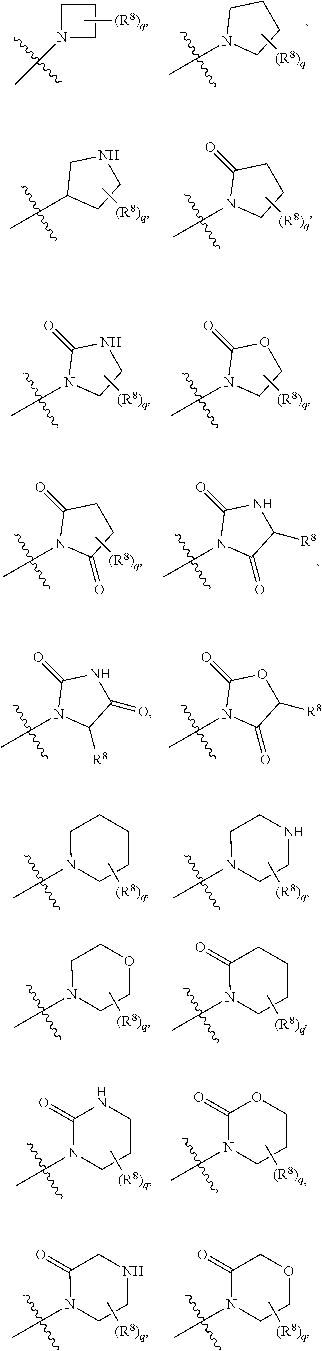 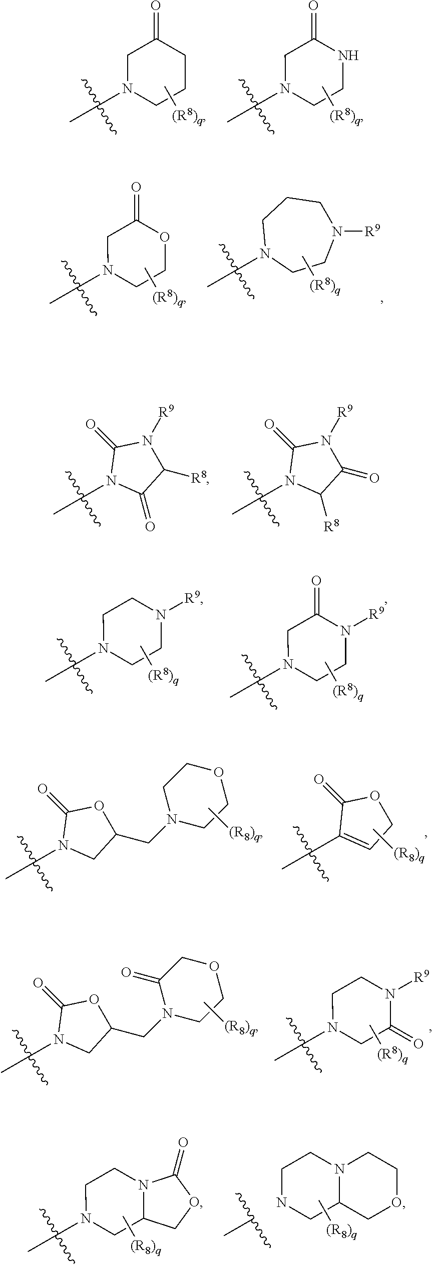 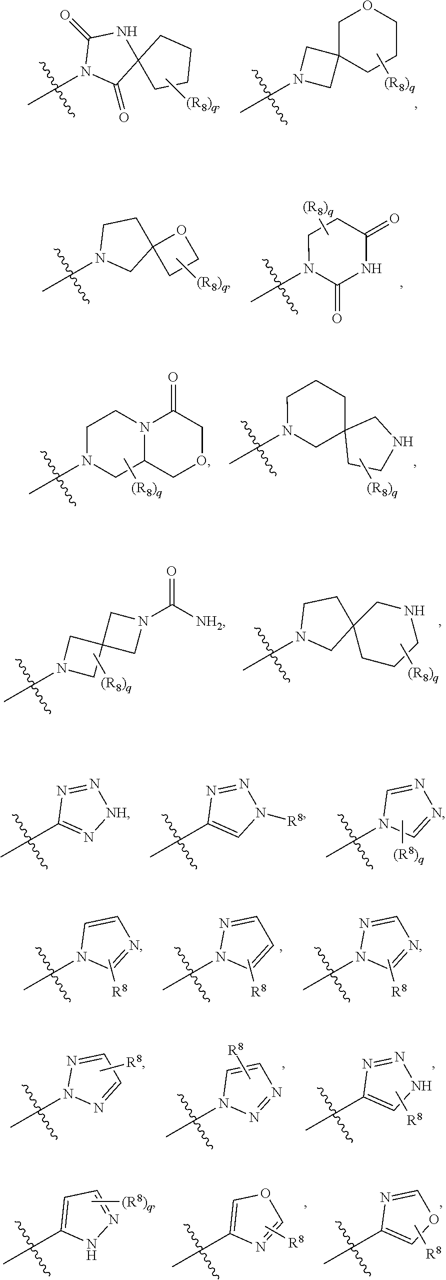 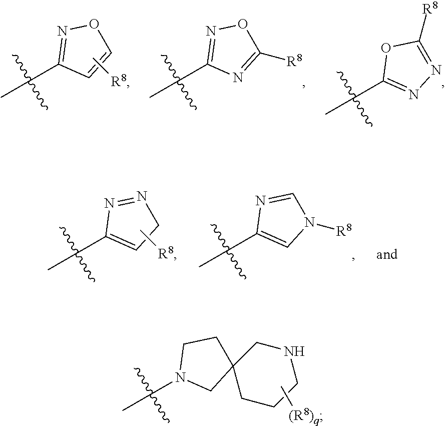
wherein q is an integer value selected from 0, 1, and 2;
wherein each R⁸ is independently selected from fluorine, phenyl, pyridine, carboxyl, —C(═O)—(C₁-C₆_alkyl), —(C₁₋₆_alkyl)-phenyl, —(C₁₋₆_alkyl)-phenyl-O—(C₁-C₆ alkyl), —CF₃, —C₁₋₆ alkyl, —(C₁₋₆ alkyl)-CN, —C₁₋₆ alkenyl, —C₃₋₈ cycloalkyl, —(C₁₋₆ alkyl)-OH, —(C₁₋₆ alkyl)-NH₂, —S(═O)₂NH₂, —S(═O)₂—(C₁₋₆ alkyl), —(C₁₋₆ alkyl)-N(CH₃)₂, —(C₁-C₆ alkyl)═O-alkyl, —(C₁-C₆ alkyl)═O—(C₁-C₆ alkyl)═OH, —(C₁₋₆ alkyl)-NH₂, C₁₋₆ alkyl-O-alkenyl, —C(═O) NH₂, —C(═O) OH, —C(═O) OCH₃, —C(═O) NH(C₁₋₆ alkyl), C(═O)N(C₁₋₆ alkyl)₂, —C(═O)—O—(C₁-6 alkyl), —(C₁₋₆ alkyl)-NC(═O)—H, —OH, —(C₁₋₆ alkyl)-NC(═O)—CF₃, or —(C₁₋₆ alkyl)-NC(═O)(C₁₋₆ alkyl); and
wherein each R⁹ is independently selected from hydrogen, —C₁₋₆ alkyl, —C₃₋₈ cycloalkyl, —(C₁₋₆ alkyl)═OH, —(C₁₋₆ alkyl)-NH₂, or —(C₁₋₆ alkyl)-NC(═O) (C₁₋₆ alkyl).

2. The compound of claim 1, wherein L¹ is O.

3. The compound of claim 1, wherein L¹ is CH₂.

4. (canceled)

5. The compound of claim 1, wherein L² is CH₂.

6. The compound of claim 1, wherein L² is CHF or CF₂.

7. (canceled)

8. The compound of Claim 1, wherein Z¹ is N, Z² is N, and Z³ is CH.

9. The compound of Claim 1, wherein Z¹ is N, Z² is CH, and Z³ is CH.

10. The compound of claim 1, wherein Z¹ is CH, Z² is CH, and Z³ is CH.

11. The compound of claim 1, wherein Z¹ is CH, Z² is CH, and Z³ is N.

12. The compound of claim 1, wherein Z¹ is CH, Z² is CH, and Z³ is CF.

13. The compound of claim 1, wherein Z¹ is N, Z² is CH, and Z³ is N.

14. The compound of claim 1, wherein Z¹ is CH, Z² is N, and Z³ is N.

15. The compound of claim 1, wherein R¹ is selected from the group consisting of —CN, halo, —C₁₋₆ alkyl, —C₁₋₆ alkoxy, —C₁₋₆ haloalkyl, and —C₁₋₆ haloalkoxy.

16. The compound of claim 1, wherein R¹ selected from the group consisting of: —CN, —F, —C₁, —Br, —CH₃, —CH₂CH3, —CF₃, —CF₂CH3, —OCF₃, —OCHF₂, and -cyclopropyl.

17. The compound of claim 1, wherein R² is selected from the group consisting of —CN, halo, —C₁₋₆ alkyl, —C₁₋₆ alkoxy, —C₁₋₆ haloalkyl, and —C₁₋₆ haloalkoxy.

18. The compound of claim 1, wherein R² is selected from the group consisting of: —CN, —F, —C₁, —Br, —CH₃, —CH₂CH3, —CF₃, —CF₂CH3, ═OCF₃, —OCHF₂, and -cyclopropyl.

19. The compound of claim 1, wherein R³, R⁴, and R⁵ are each independently is-hydrogen or halo.

20. (canceled)

21. (canceled)

22. (canceled)

23. The compound of Claim 1, wherein R⁶ is selected from the group consisting of:

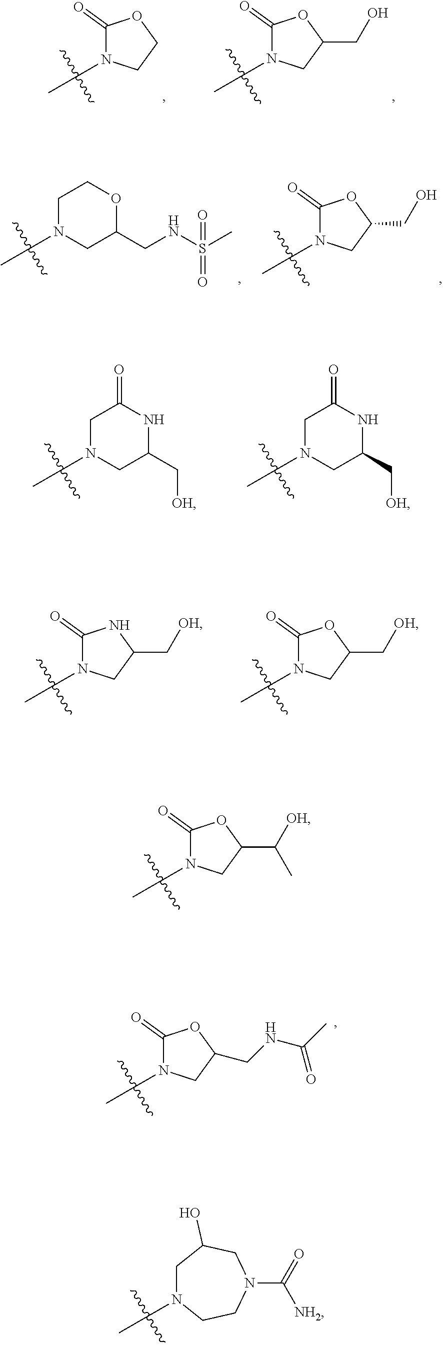

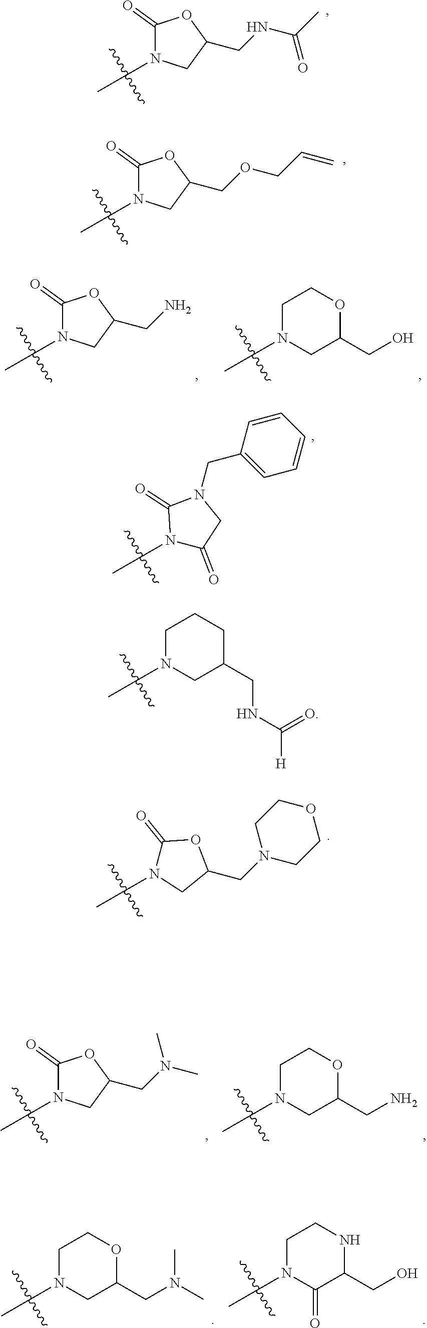

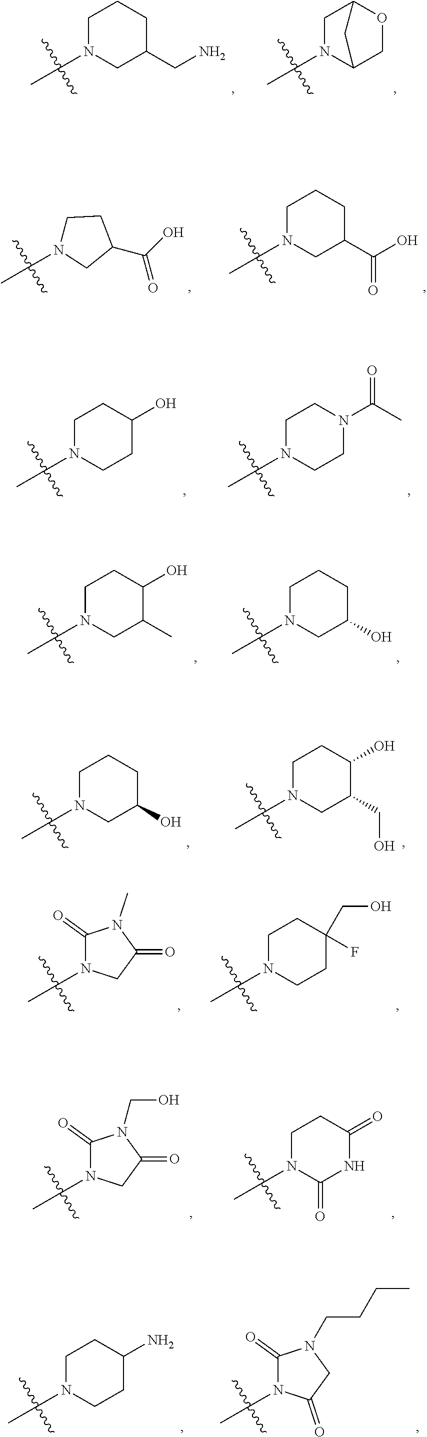

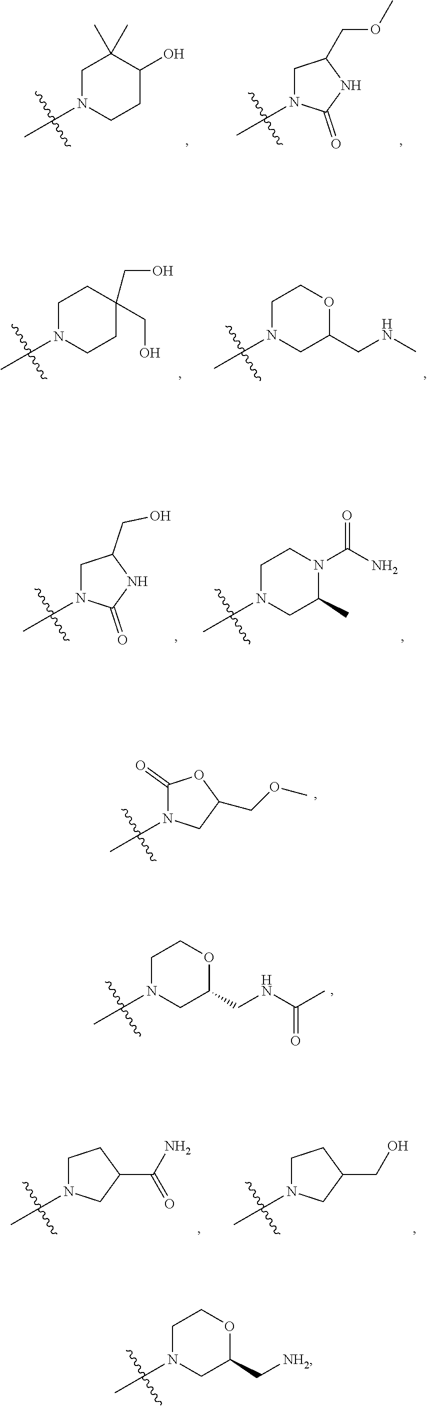

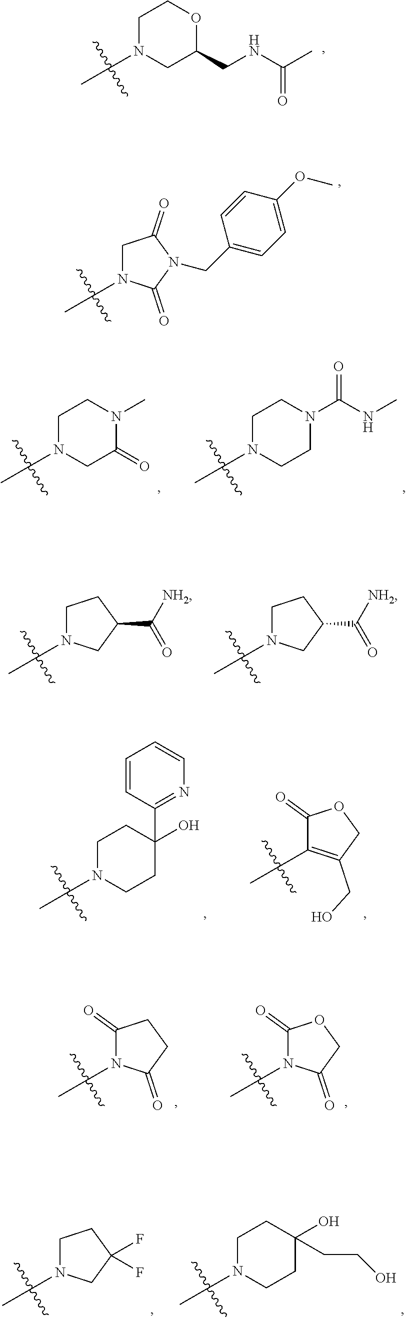

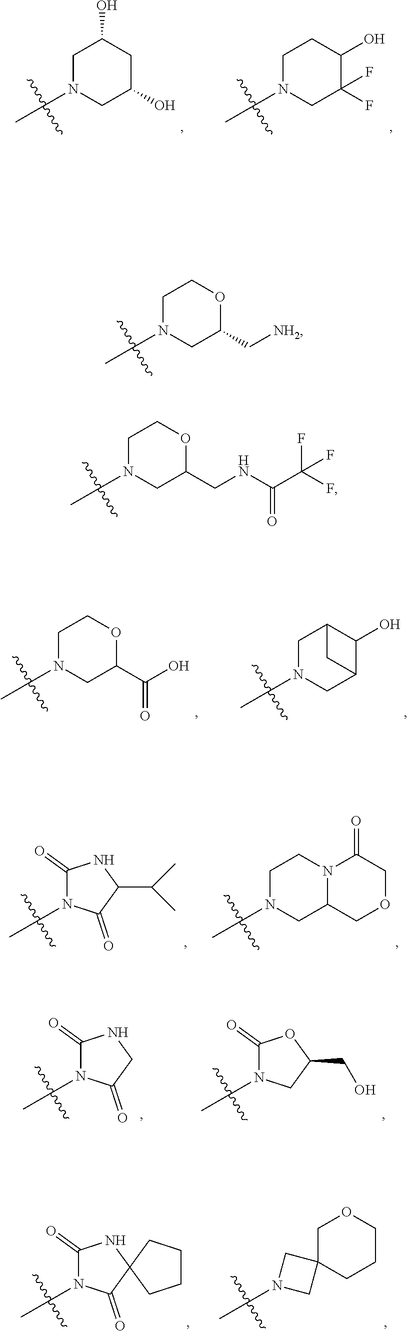

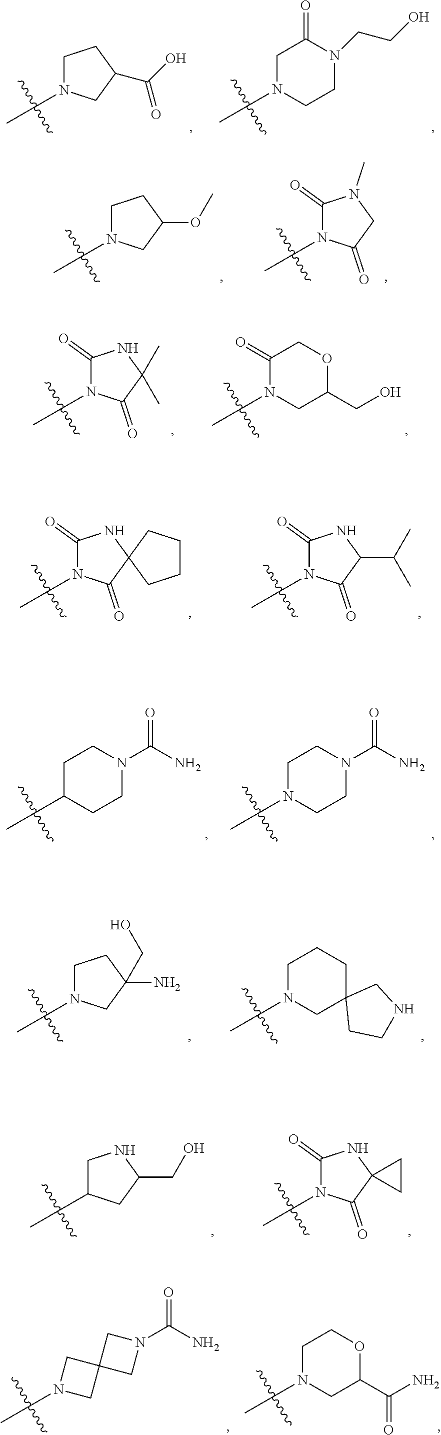

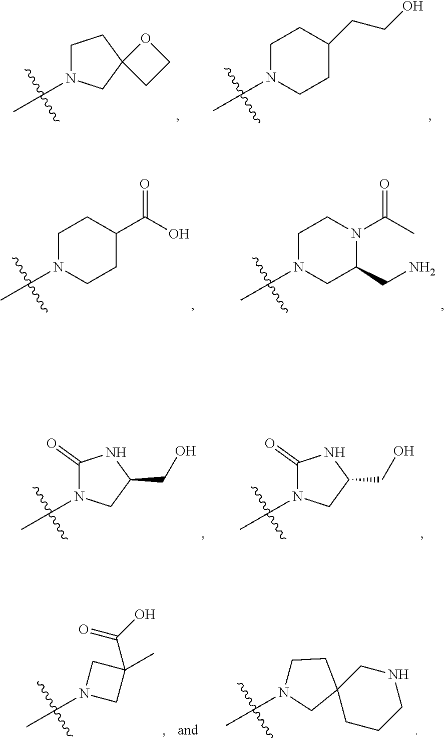

24. A compound selected from the group consisting of: 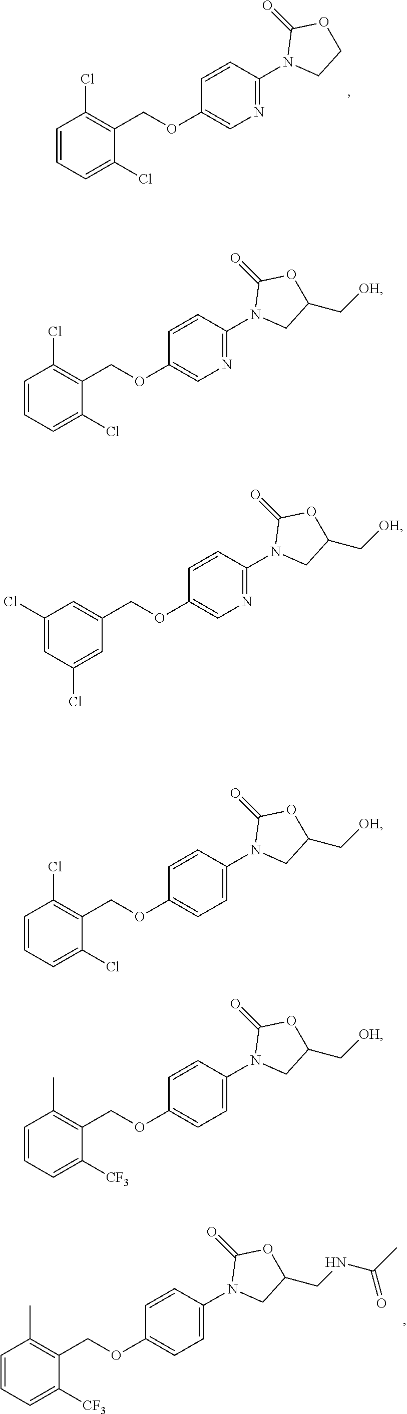 [SMILES: O=C1OCCN1c1ccc(OCc2c(Cl)cccc2Cl)cn1 ; O=C1OC(CO)CN1c1ccc(OCc2c(Cl)cccc2Cl)cn1 ; O=C1OC(CO)CN1c1ccc(OCc2cc(Cl)cc(Cl)c2)cn1 ; O=C1OC(CO)CN1c1ccc(OCc2c(Cl)cccc2Cl)cc1 ; Cc1cccc(C(F)(F)F)c1COc1ccc(N2CC(CO)OC2=O)cc1 ; CC(=O)NCC1CN(c2ccc(OCc3c(C)cccc3C(F)(F)F)cc2)C(=O)O1] 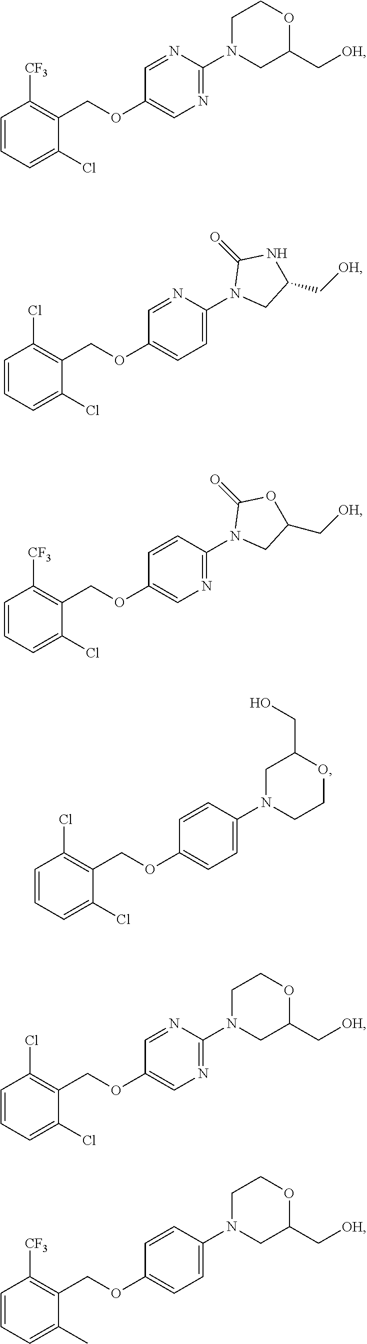 [SMILES: OCC1CN(c2ncc(OCc3c(Cl)cccc3C(F)(F)F)cn2)CCO1 ; O=C1N[C@H](CO)CN1c1ccc(OCc2c(Cl)cccc2Cl)cn1 ; O=C1OC(CO)CN1c1ccc(OCc2c(Cl)cccc2C(F)(F)F)cn1 ; OCC1CN(c2ccc(OCc3c(Cl)cccc3Cl)cc2)CCO1 ; OCC1CN(c2ncc(OCc3c(Cl)cccc3Cl)cn2)CCO1 ; Cc1cccc(C(F)(F)F)c1COc1ccc(N2CCOC(CO)C2)cc1] 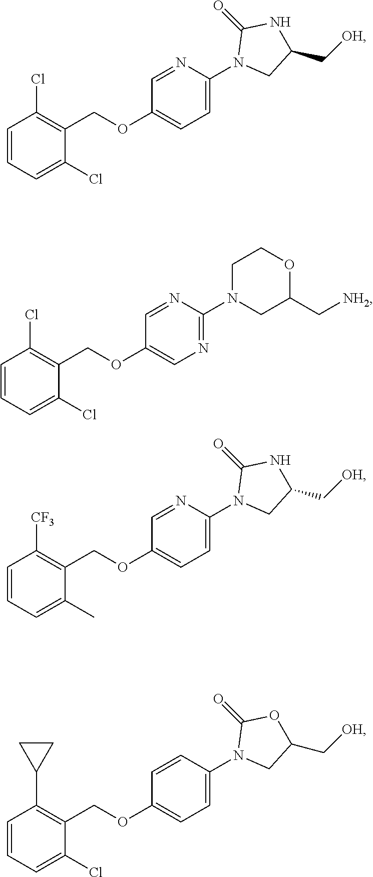 [SMILES: O=C1N[C@@H](CO)CN1c1ccc(OCc2c(Cl)cccc2Cl)cn1 ; NCC1CN(c2ncc(OCc3c(Cl)cccc3Cl)cn2)CCO1 ; Cc1cccc(C(F)(F)F)c1COc1ccc(N2C[C@@H](CO)NC2=O)nc1 ; O=C1OC(CO)CN1c1ccc(OCc2c(Cl)cccc2C2CC2)cc1] 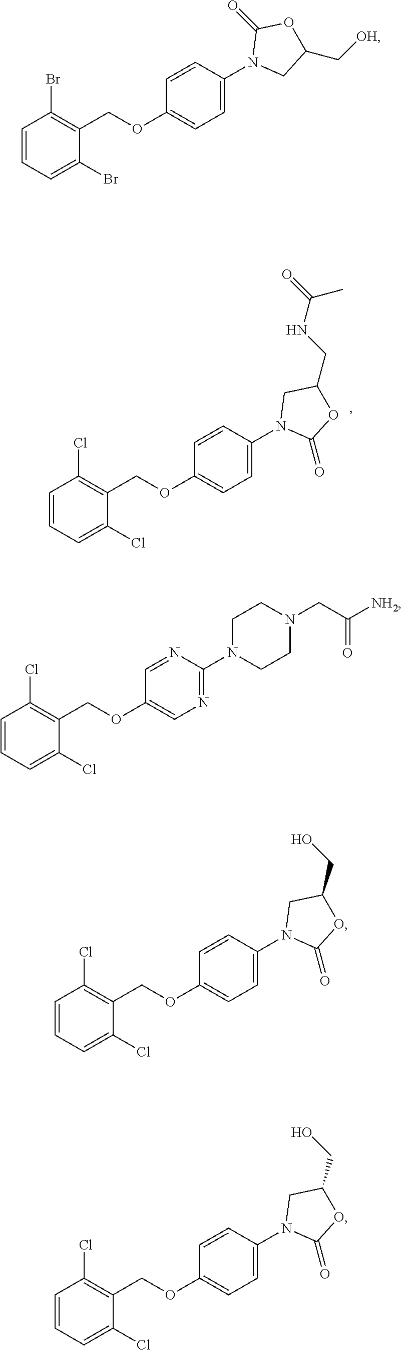 [SMILES: O=C1OC(CO)CN1c1ccc(OCc2c(Br)cccc2Br)cc1 ; CC(=O)NCC1CN(c2ccc(OCc3c(Cl)cccc3Cl)cc2)C(=O)O1 ; NC(=O)CN1CCN(c2ncc(OCc3c(Cl)cccc3Cl)cn2)CC1 ; O=C1O[C@H](CO)CN1c1ccc(OCc2c(Cl)cccc2Cl)cc1 ; O=C1O[C@@H](CO)CN1c1ccc(OCc2c(Cl)cccc2Cl)cc1] 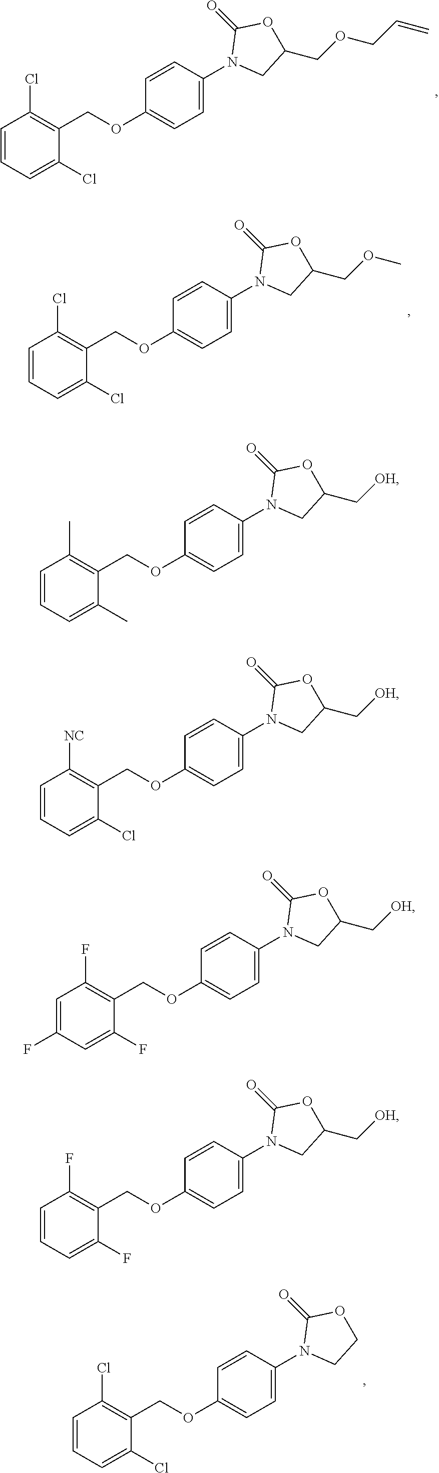 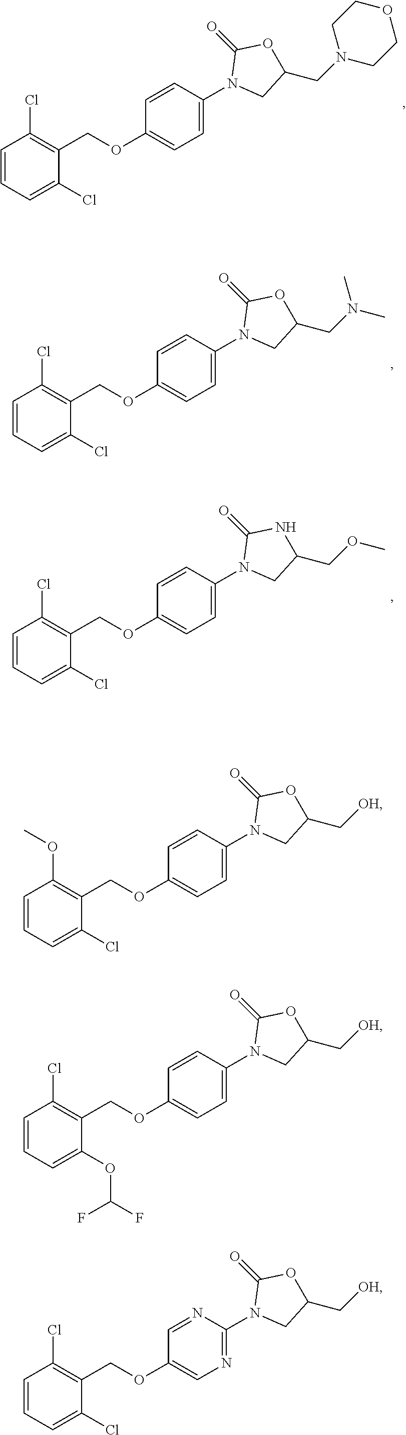 [SMILES: O=C1OC(CN2CCOCC2)CN1c1ccc(OCc2c(Cl)cccc2Cl)cc1 ; CN(C)CC1CN(c2ccc(OCc3c(Cl)cccc3Cl)cc2)C(=O)O1 ; COCC1CN(c2ccc(OCc3c(Cl)cccc3Cl)cc2)C(=O)N1 ; COc1cccc(Cl)c1COc1ccc(N2CC(CO)OC2=O)cc1 ; O=C1OC(CO)CN1c1ccc(OCc2c(Cl)cccc2OC(F)F)cc1 ; O=C1OC(CO)CN1c1ncc(OCc2c(Cl)cccc2Cl)cn1] 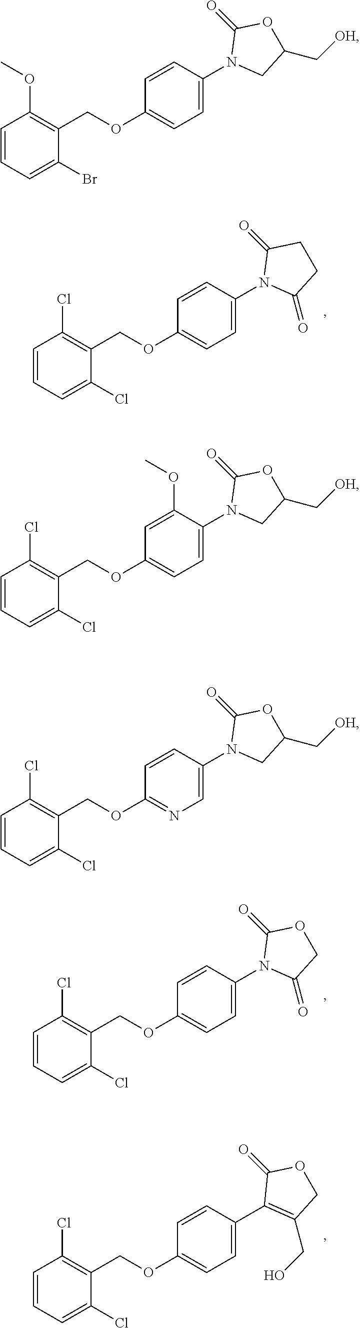 [SMILES: COc1cccc(Br)c1COc1ccc(N2CC(CO)OC2=O)cc1 ; O=C1CCC(=O)N1c1ccc(OCc2c(Cl)cccc2Cl)cc1 ; COc1cc(OCc2c(Cl)cccc2Cl)ccc1N1CC(CO)OC1=O ; O=C1OC(CO)CN1c1ccc(OCc2c(Cl)cccc2Cl)nc1 ; O=C1COC(=O)N1c1ccc(OCc2c(Cl)cccc2Cl)cc1 ; O=C1OCC(CO)=C1c1ccc(OCc2c(Cl)cccc2Cl)cc1] 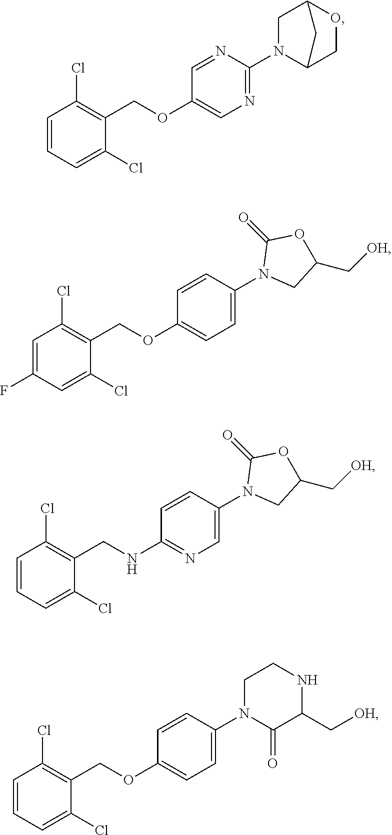 [SMILES: Clc1cccc(Cl)c1COc1cnc(N2CC3CC2CO3)nc1 ; O=C1OC(CO)CN1c1ccc(OCc2c(Cl)cc(F)cc2Cl)cc1 ; O=C1OC(CO)CN1c1ccc(NCc2c(Cl)cccc2Cl)nc1 ; O=C1C(CO)NCCN1c1ccc(OCc2c(Cl)cccc2Cl)cc1] 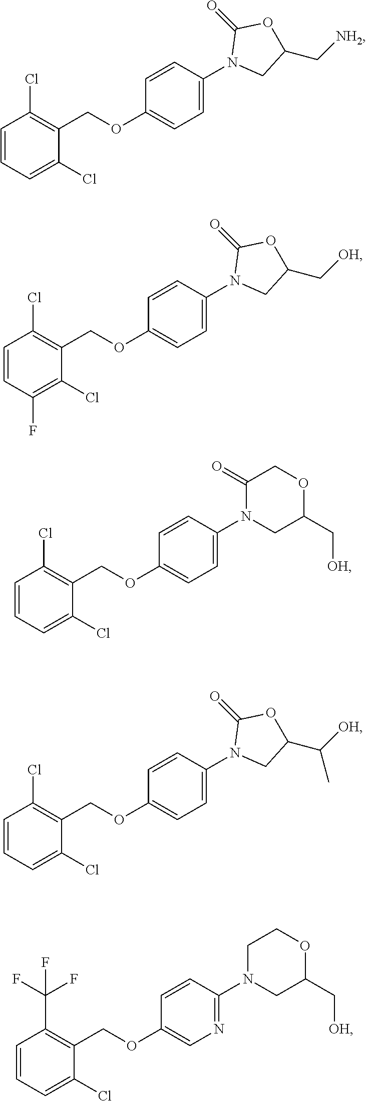 [SMILES: NCC1CN(c2ccc(OCc3c(Cl)cccc3Cl)cc2)C(=O)O1 ; O=C1OC(CO)CN1c1ccc(OCc2c(Cl)ccc(F)c2Cl)cc1 ; O=C1COC(CO)CN1c1ccc(OCc2c(Cl)cccc2Cl)cc1 ; CC(O)C1CN(c2ccc(OCc3c(Cl)cccc3Cl)cc2)C(=O)O1 ; OCC1CN(c2ccc(OCc3c(Cl)cccc3C(F)(F)F)cn2)CCO1] 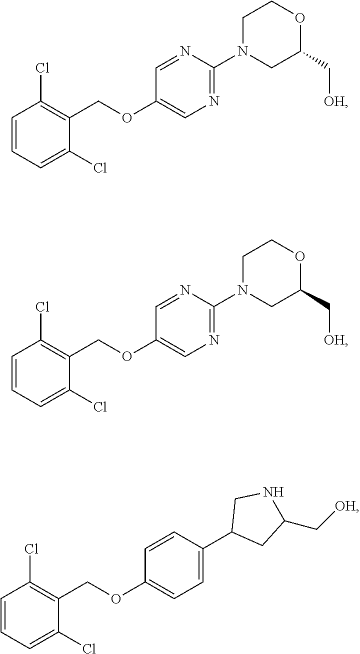 [SMILES: OC[C@@H]1CN(c2ncc(OCc3c(Cl)cccc3Cl)cn2)CCO1 ; OC[C@H]1CN(c2ncc(OCc3c(Cl)cccc3Cl)cn2)CCO1 ; OCC1CC(c2ccc(OCc3c(Cl)cccc3Cl)cc2)CN1] 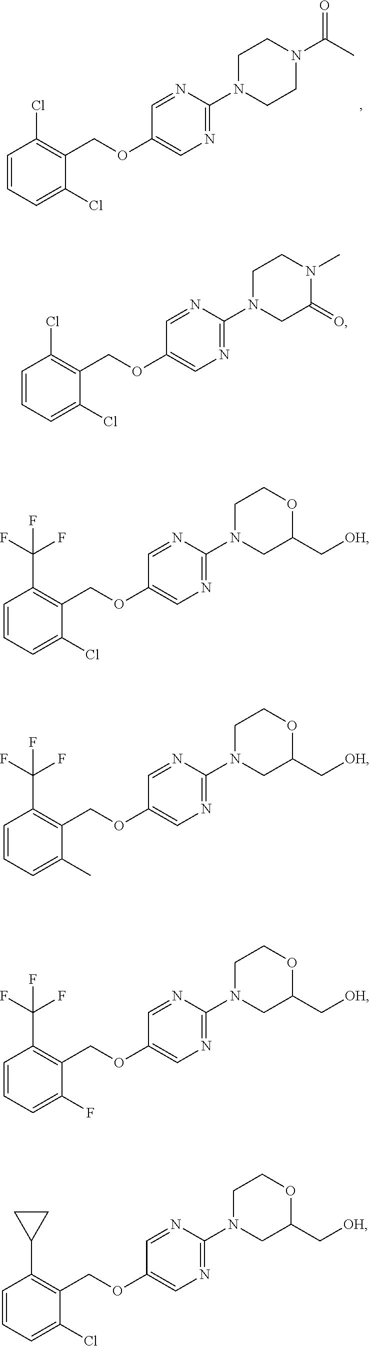 [SMILES: CC(=O)N1CCN(c2ncc(OCc3c(Cl)cccc3Cl)cn2)CC1 ; CN1CCN(c2ncc(OCc3c(Cl)cccc3Cl)cn2)CC1=O ; OCC1CN(c2ncc(OCc3c(Cl)cccc3C(F)(F)F)cn2)CCO1 ; Cc1cccc(C(F)(F)F)c1COc1cnc(N2CCOC(CO)C2)nc1 ; OCC1CN(c2ncc(OCc3c(F)cccc3C(F)(F)F)cn2)CCO1 ; OCC1CN(c2ncc(OCc3c(Cl)cccc3C3CC3)cn2)CCO1] 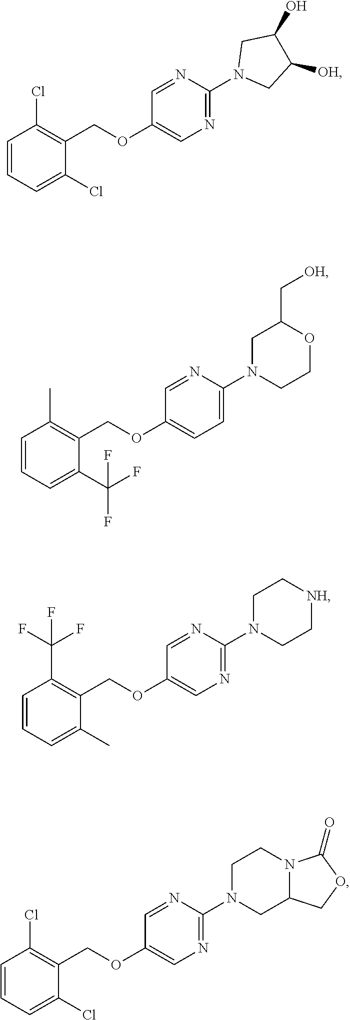 [SMILES: O[C@@H]1CN(c2ncc(OCc3c(Cl)cccc3Cl)cn2)C[C@@H]1O ; Cc1cccc(C(F)(F)F)c1COc1ccc(N2CCOC(CO)C2)nc1 ; Cc1cccc(C(F)(F)F)c1COc1cnc(N2CCNCC2)nc1 ; O=C1OCC2CN(c3ncc(OCc4c(Cl)cccc4Cl)cn3)CCN12] 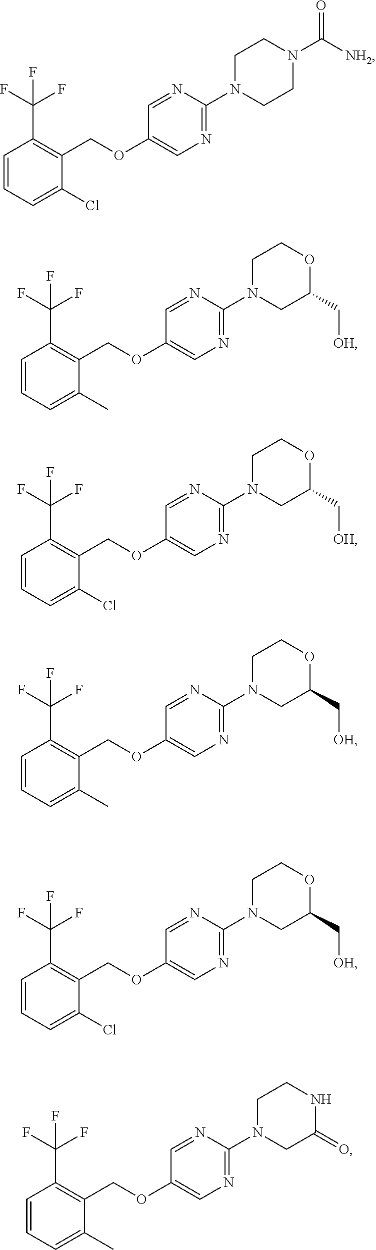 [SMILES: NC(=O)N1CCN(c2ncc(OCc3c(Cl)cccc3C(F)(F)F)cn2)CC1 ; Cc1cccc(C(F)(F)F)c1COc1cnc(N2CCO[C@H](CO)C2)nc1 ; OC[C@@H]1CN(c2ncc(OCc3c(Cl)cccc3C(F)(F)F)cn2)CCO1 ; Cc1cccc(C(F)(F)F)c1COc1cnc(N2CCO[C@@H](CO)C2)nc1 ; OC[C@H]1CN(c2ncc(OCc3c(Cl)cccc3C(F)(F)F)cn2)CCO1 ; Cc1cccc(C(F)(F)F)c1COc1cnc(N2CCNC(=O)C2)nc1] 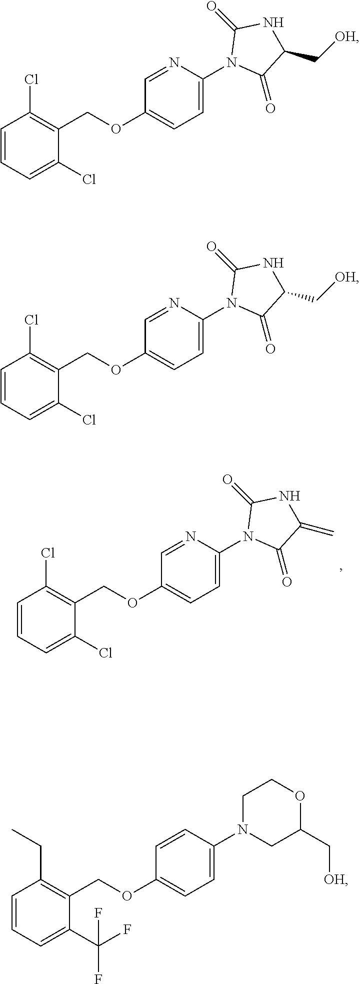 [SMILES: O=C1N[C@@H](CO)C(=O)N1c1ccc(OCc2c(Cl)cccc2Cl)cn1 ; O=C1N[C@H](CO)C(=O)N1c1ccc(OCc2c(Cl)cccc2Cl)cn1 ; C=C1NC(=O)N(c2ccc(OCc3c(Cl)cccc3Cl)cn2)C1=O ; CCc1cccc(C(F)(F)F)c1COc1ccc(N2CCOC(CO)C2)cc1] 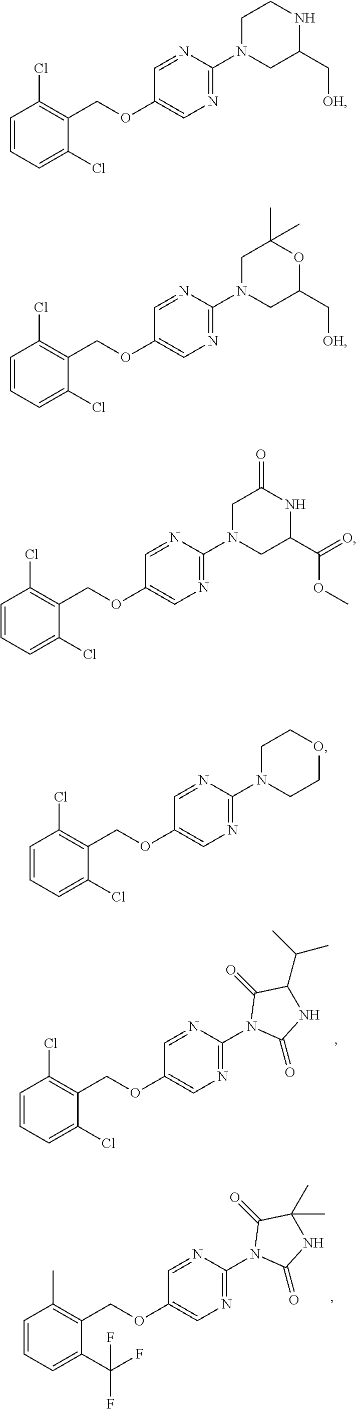 [SMILES: OCC1CN(c2ncc(OCc3c(Cl)cccc3Cl)cn2)CCN1 ; CC1(C)CN(c2ncc(OCc3c(Cl)cccc3Cl)cn2)CC(CO)O1 ; COC(=O)C1CN(c2ncc(OCc3c(Cl)cccc3Cl)cn2)CC(=O)N1 ; Clc1cccc(Cl)c1COc1cnc(N2CCOCC2)nc1 ; CC(C)C1NC(=O)N(c2ncc(OCc3c(Cl)cccc3Cl)cn2)C1=O ; Cc1cccc(C(F)(F)F)c1COc1cnc(N2C(=O)NC(C)(C)C2=O)nc1] 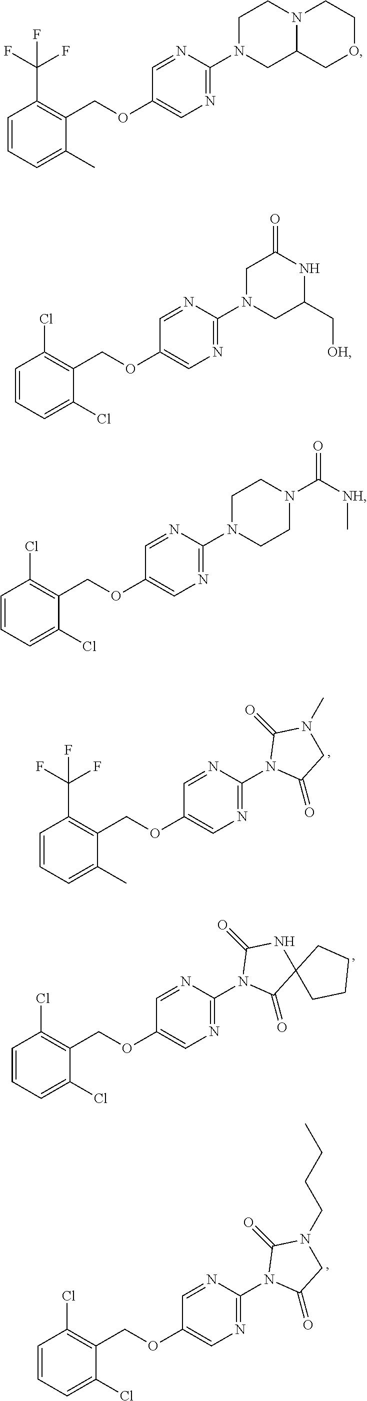 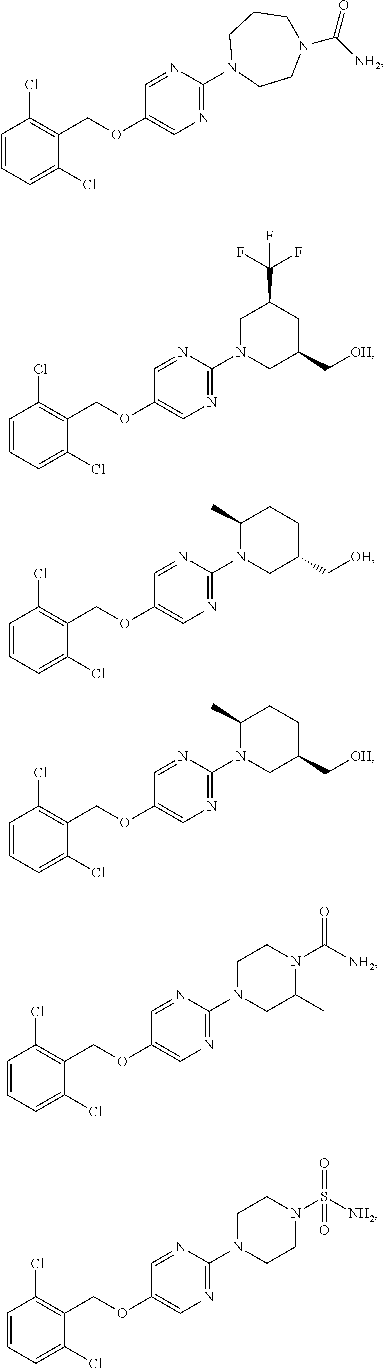 [SMILES: NC(=O)N1CCCN(c2ncc(OCc3c(Cl)cccc3Cl)cn2)CC1 ; OC[C@@H]1C[C@H](C(F)(F)F)CN(c2ncc(OCc3c(Cl)cccc3Cl)cn2)C1 ; C[C@H]1CC[C@H](CO)CN1c1ncc(OCc2c(Cl)cccc2Cl)cn1 ; C[C@H]1CC[C@@H](CO)CN1c1ncc(OCc2c(Cl)cccc2Cl)cn1 ; CC1CN(c2ncc(OCc3c(Cl)cccc3Cl)cn2)CCN1C(N)=O ; NS(=O)(=O)N1CCN(c2ncc(OCc3c(Cl)cccc3Cl)cn2)CC1] 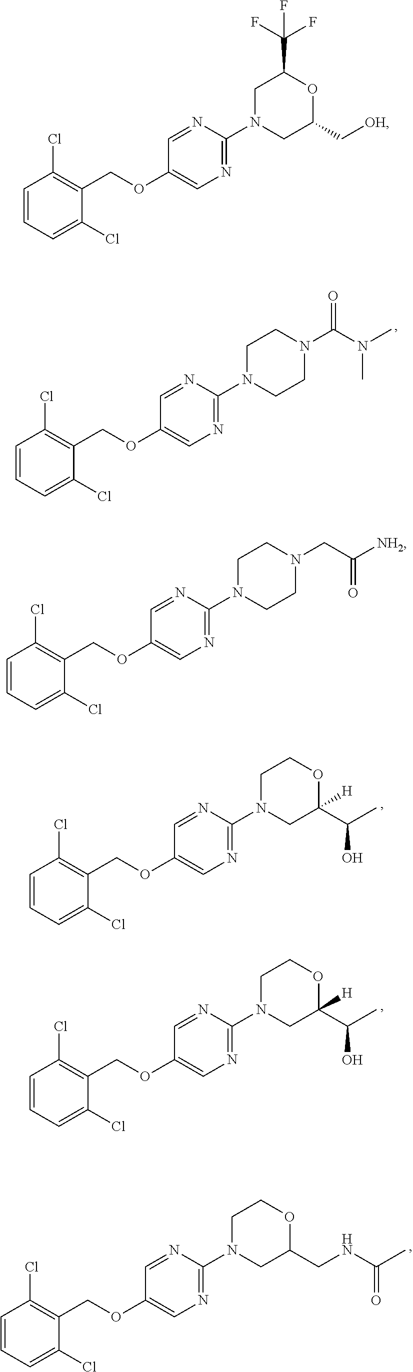 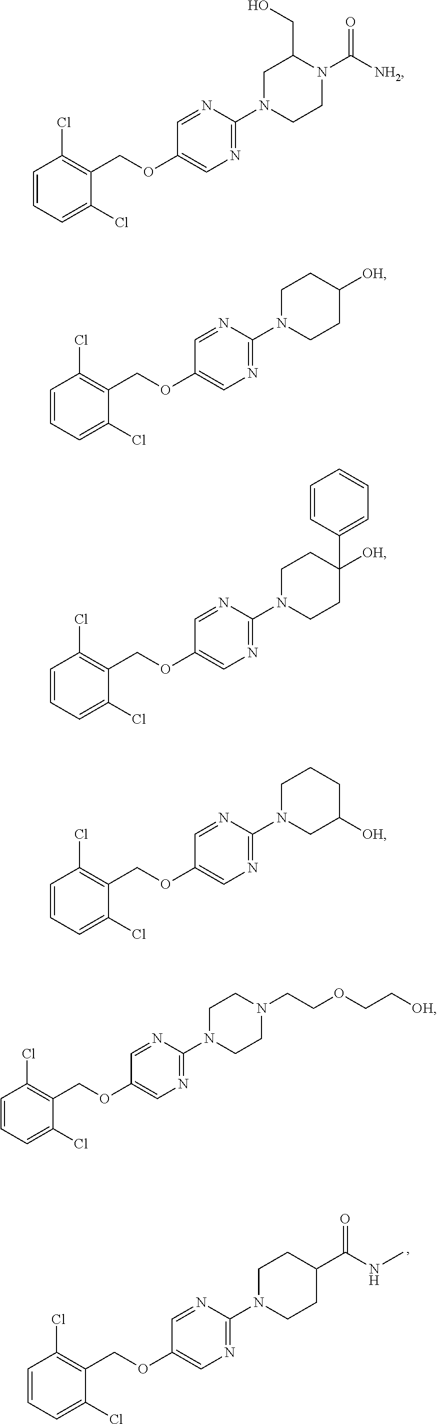 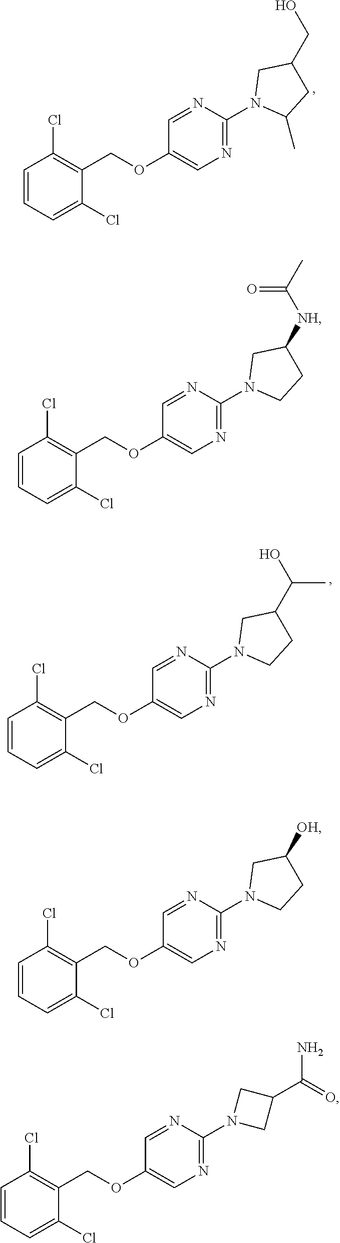 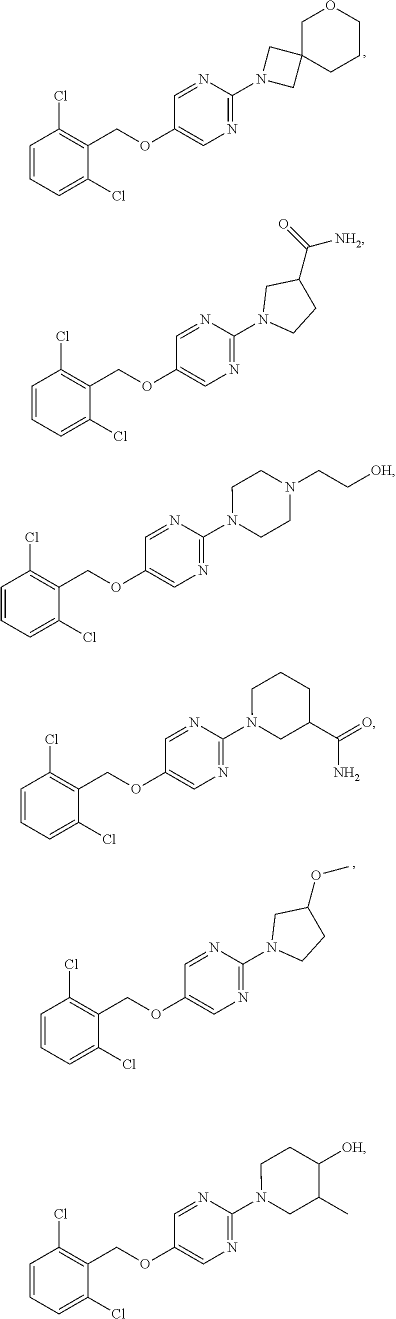 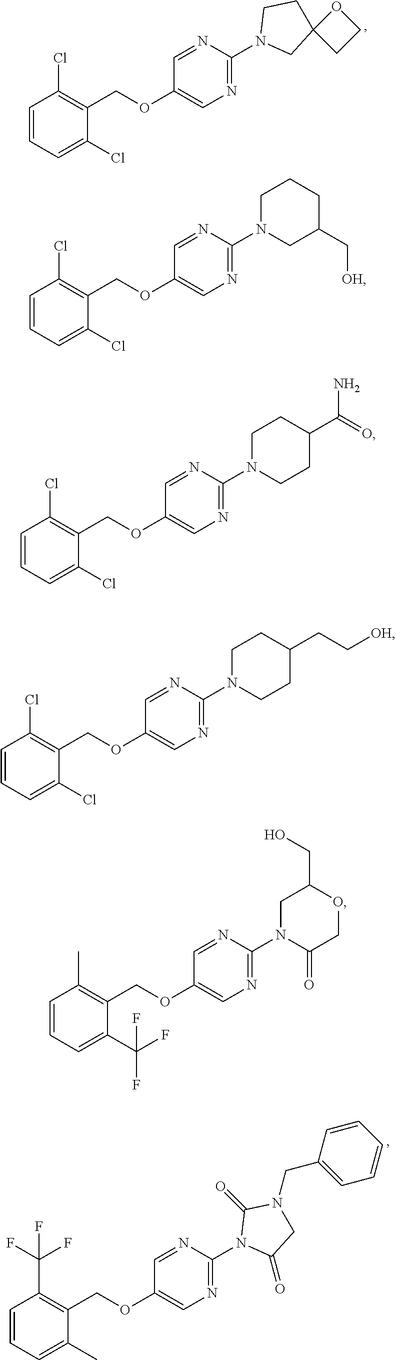 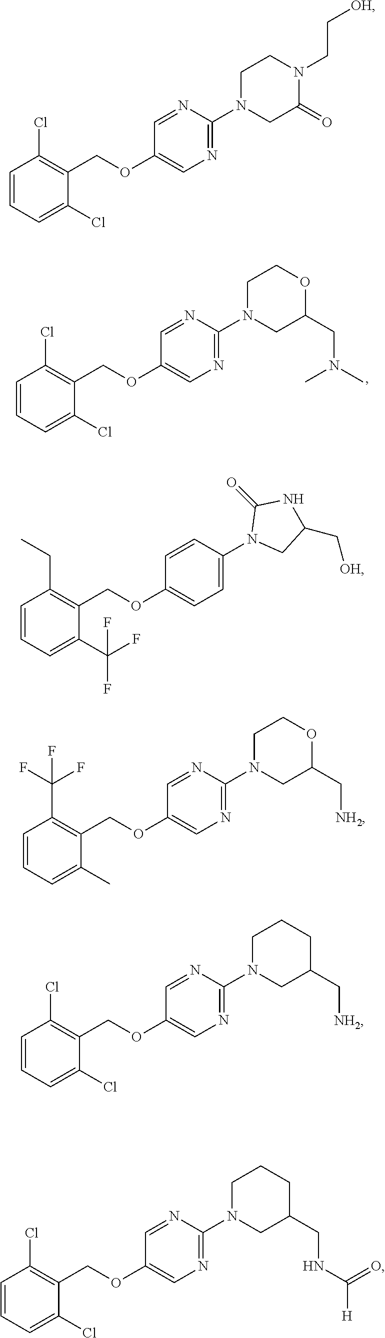 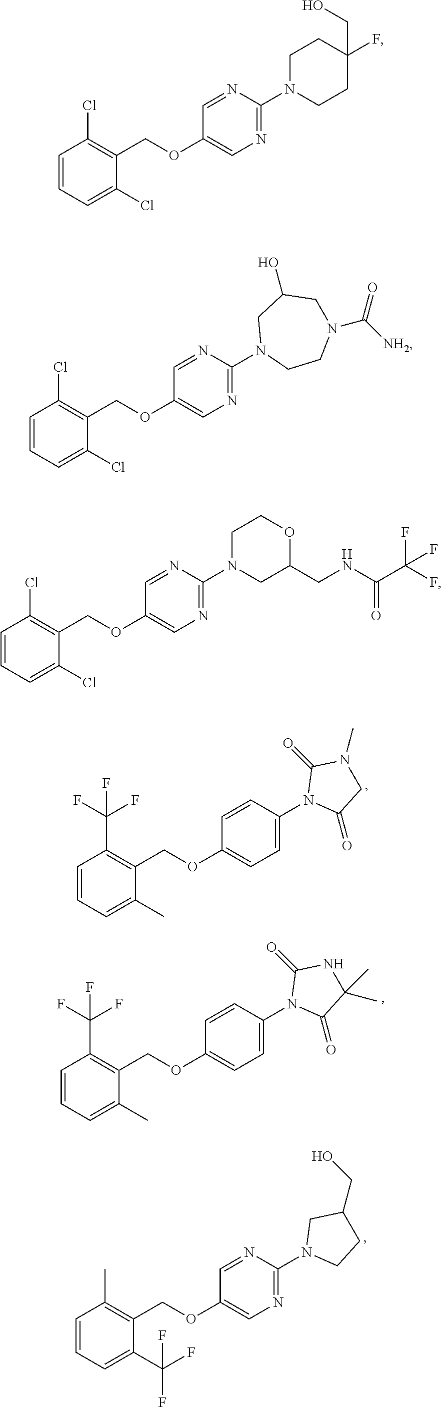 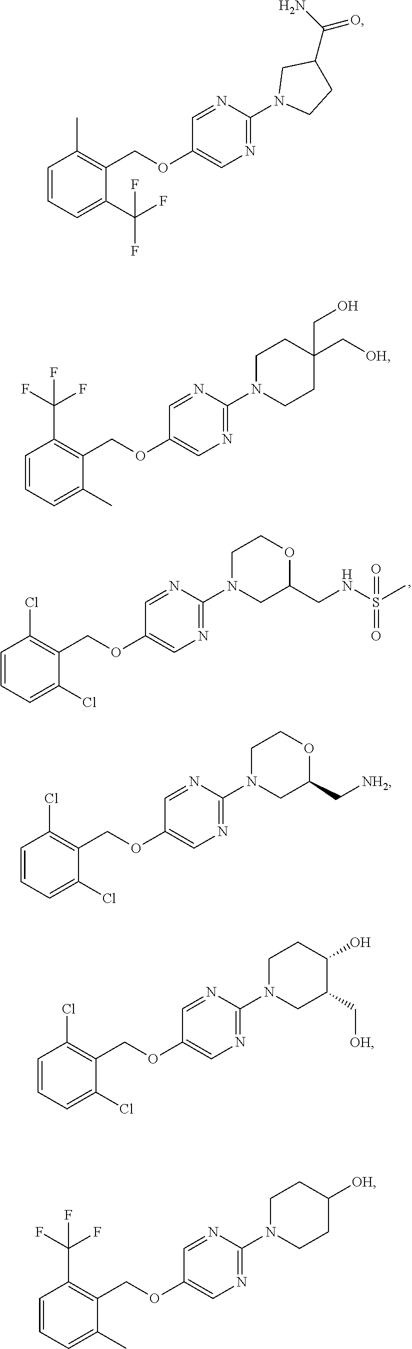 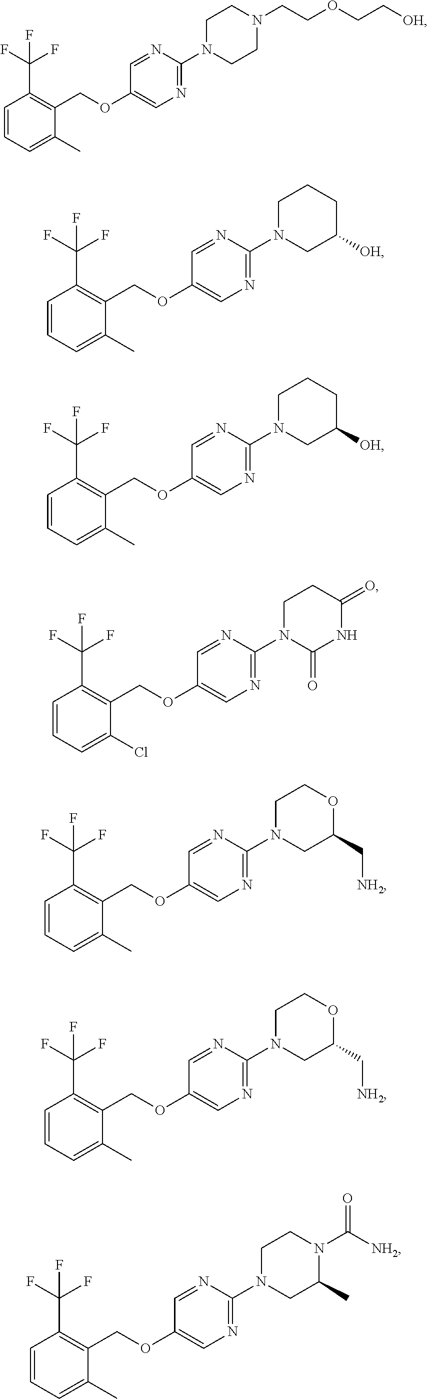 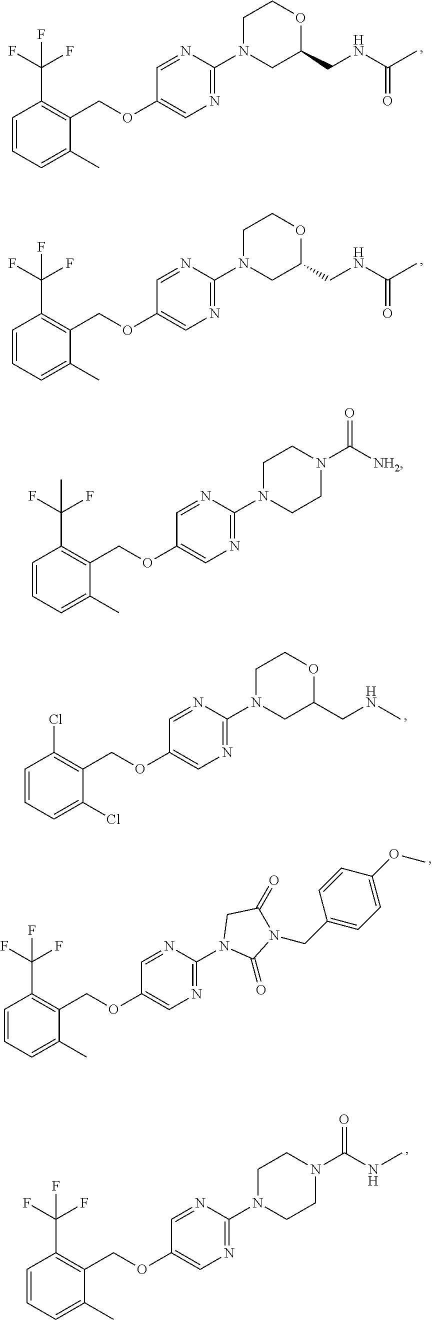 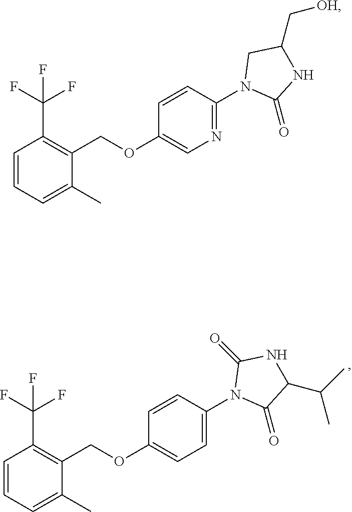 [SMILES: Cc1cccc(C(F)(F)F)c1COc1ccc(N2CC(CO)NC2=O)nc1 ; Cc1cccc(C(F)(F)F)c1COc1ccc(N2C(=O)NC(C(C)C)C2=O)cc1 ; ","] 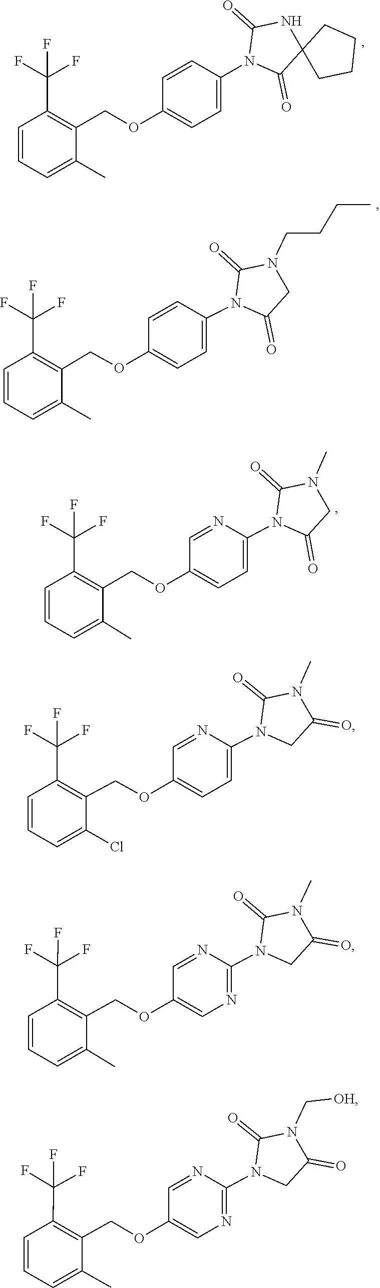 [SMILES: Cc1cccc(C(F)(F)F)c1COc1ccc(N2C(=O)NC3(CCCC3)C2=O)cc1 ; CCCCN1CC(=O)N(c2ccc(OCc3c(C)cccc3C(F)(F)F)cc2)C1=O ; Cc1cccc(C(F)(F)F)c1COc1ccc(N2C(=O)CN(C)C2=O)nc1 ; CN1C(=O)CN(c2ccc(OCc3c(Cl)cccc3C(F)(F)F)cn2)C1=O ; Cc1cccc(C(F)(F)F)c1COc1cnc(N2CC(=O)N(C)C2=O)nc1 ; Cc1cccc(C(F)(F)F)c1COc1cnc(N2CC(=O)N(CO)C2=O)nc1] 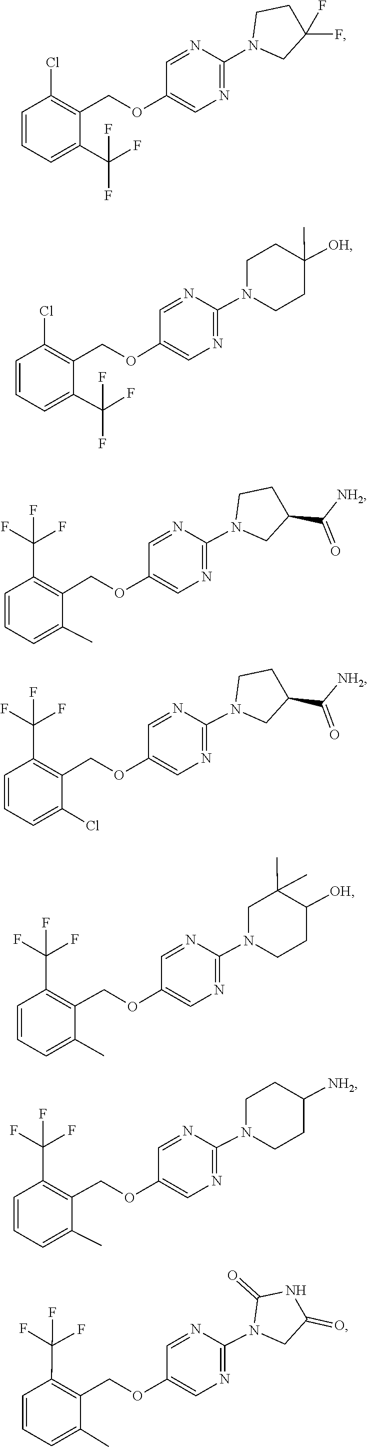 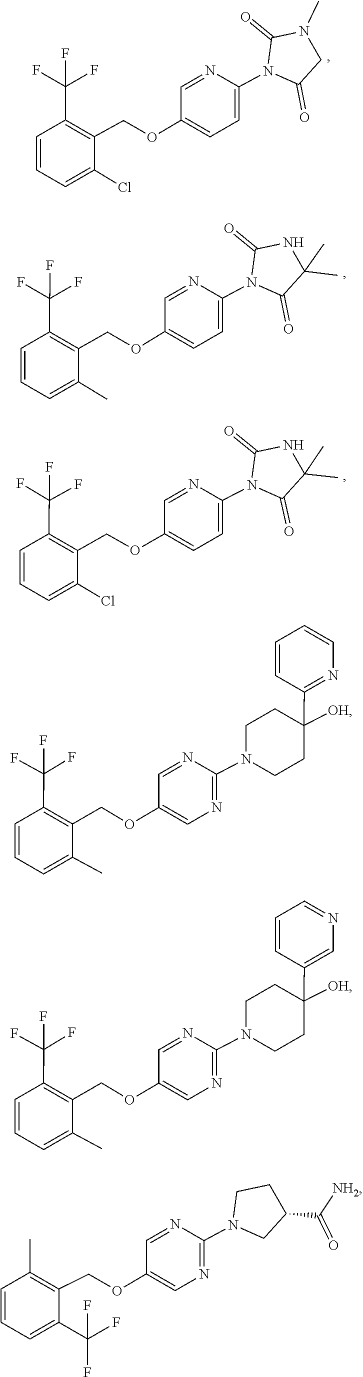 [SMILES: CN1CC(=O)N(c2ccc(OCc3c(Cl)cccc3C(F)(F)F)cn2)C1=O ; Cc1cccc(C(F)(F)F)c1COc1ccc(N2C(=O)NC(C)(C)C2=O)nc1 ; CC1(C)NC(=O)N(c2ccc(OCc3c(Cl)cccc3C(F)(F)F)cn2)C1=O ; Cc1cccc(C(F)(F)F)c1COc1cnc(N2CCC(O)(c3ccccn3)CC2)nc1 ; Cc1cccc(C(F)(F)F)c1COc1cnc(N2CCC(O)(c3cccnc3)CC2)nc1 ; Cc1cccc(C(F)(F)F)c1COc1cnc(N2CC[C@H](C(N)=O)C2)nc1] 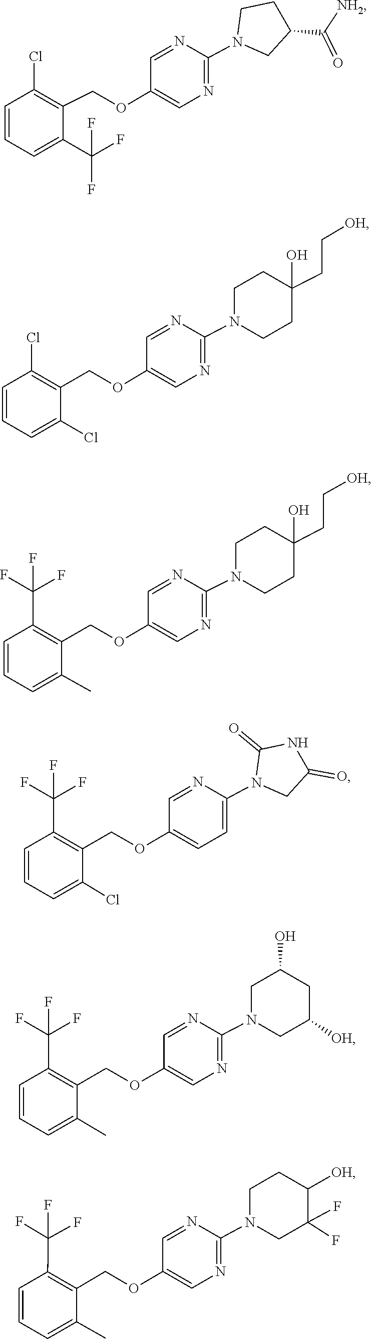 [SMILES: NC(=O)[C@H]1CCN(c2ncc(OCc3c(Cl)cccc3C(F)(F)F)cn2)C1 ; OCCC1(O)CCN(c2ncc(OCc3c(Cl)cccc3Cl)cn2)CC1 ; Cc1cccc(C(F)(F)F)c1COc1cnc(N2CCC(O)(CCO)CC2)nc1 ; O=C1CN(c2ccc(OCc3c(Cl)cccc3C(F)(F)F)cn2)C(=O)N1 ; Cc1cccc(C(F)(F)F)c1COc1cnc(N2C[C@H](O)C[C@H](O)C2)nc1 ; Cc1cccc(C(F)(F)F)c1COc1cnc(N2CCC(O)C(F)(F)C2)nc1] 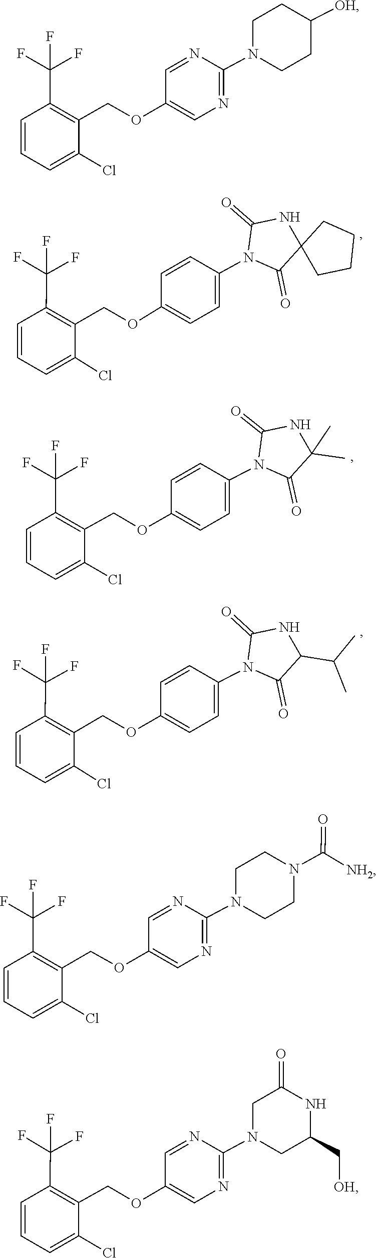 [SMILES: OC1CCN(c2ncc(OCc3c(Cl)cccc3C(F)(F)F)cn2)CC1 ; O=C1NC2(CCCC2)C(=O)N1c1ccc(OCc2c(Cl)cccc2C(F)(F)F)cc1 ; CC1(C)NC(=O)N(c2ccc(OCc3c(Cl)cccc3C(F)(F)F)cc2)C1=O ; CC(C)C1NC(=O)N(c2ccc(OCc3c(Cl)cccc3C(F)(F)F)cc2)C1=O ; NC(=O)N1CCN(c2ncc(OCc3c(Cl)cccc3C(F)(F)F)cn2)CC1 ; O=C1CN(c2ncc(OCc3c(Cl)cccc3C(F)(F)F)cn2)C[C@H](CO)N1] 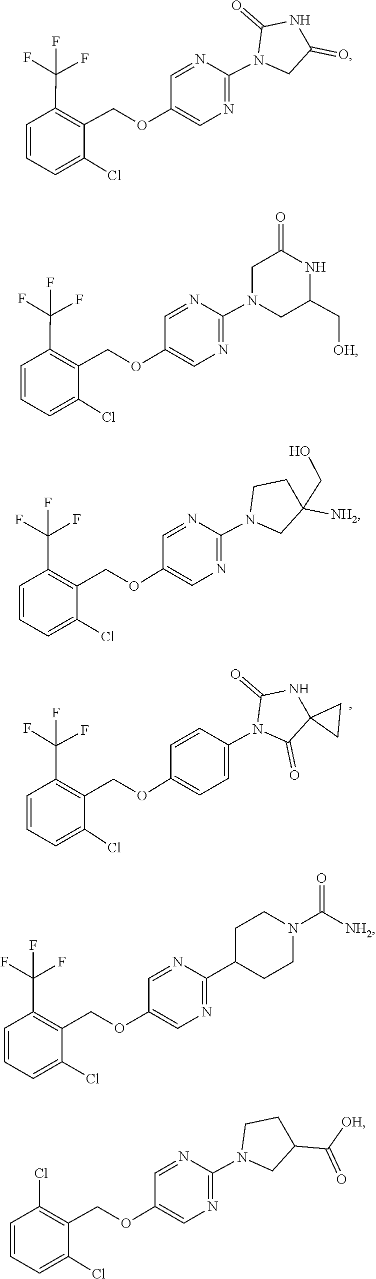 [SMILES: O=C1CN(c2ncc(OCc3c(Cl)cccc3C(F)(F)F)cn2)C(=O)N1 ; O=C1CN(c2ncc(OCc3c(Cl)cccc3C(F)(F)F)cn2)CC(CO)N1 ; NC1(CO)CCN(c2ncc(OCc3c(Cl)cccc3C(F)(F)F)cn2)C1 ; O=C1NC2(CC2)C(=O)N1c1ccc(OCc2c(Cl)cccc2C(F)(F)F)cc1 ; NC(=O)N1CCC(c2ncc(OCc3c(Cl)cccc3C(F)(F)F)cn2)CC1 ; O=C(O)C1CCN(c2ncc(OCc3c(Cl)cccc3Cl)cn2)C1] 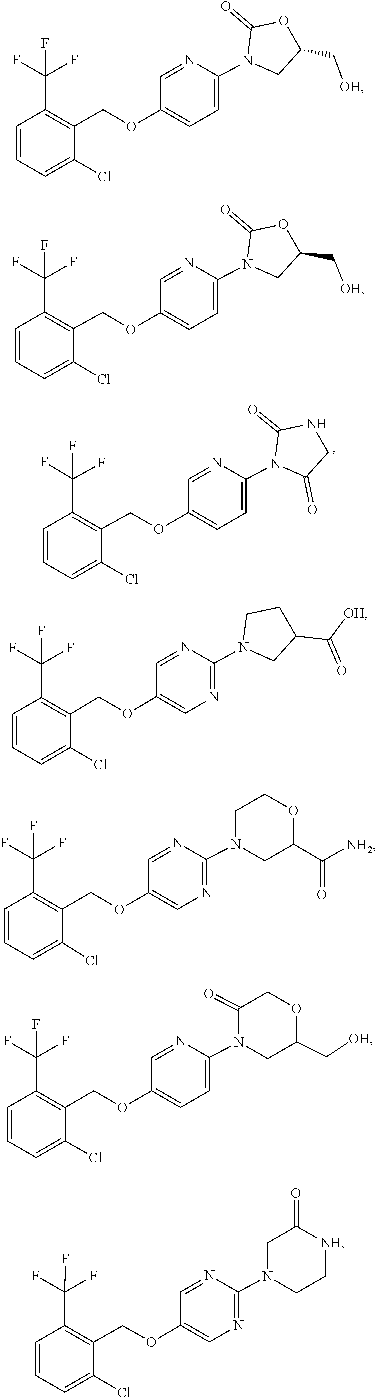 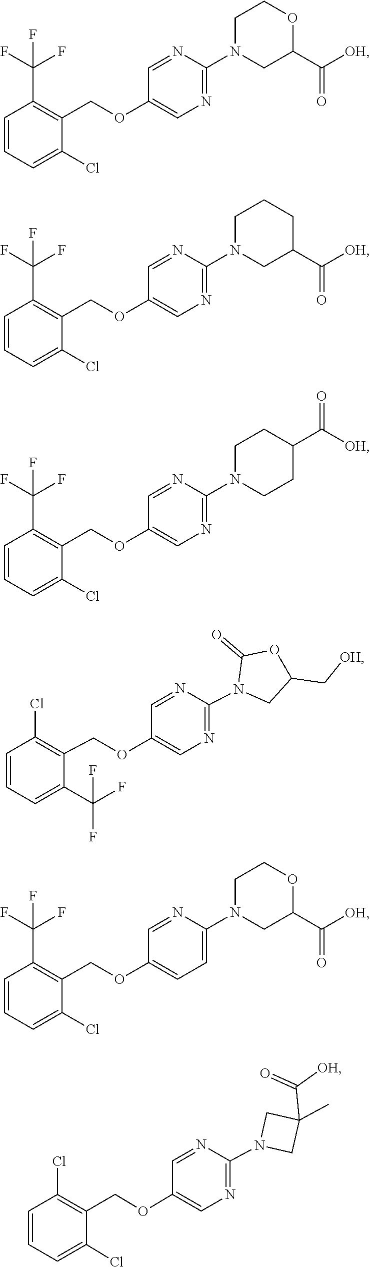 [SMILES: O=C(O)C1CN(c2ncc(OCc3c(Cl)cccc3C(F)(F)F)cn2)CCO1 ; O=C(O)C1CCCN(c2ncc(OCc3c(Cl)cccc3C(F)(F)F)cn2)C1 ; O=C(O)C1CCN(c2ncc(OCc3c(Cl)cccc3C(F)(F)F)cn2)CC1 ; O=C1OC(CO)CN1c1ncc(OCc2c(Cl)cccc2C(F)(F)F)cn1 ; O=C(O)C1CN(c2ccc(OCc3c(Cl)cccc3C(F)(F)F)cn2)CCO1 ; CC1(C(=O)O)CN(c2ncc(OCc3c(Cl)cccc3Cl)cn2)C1] 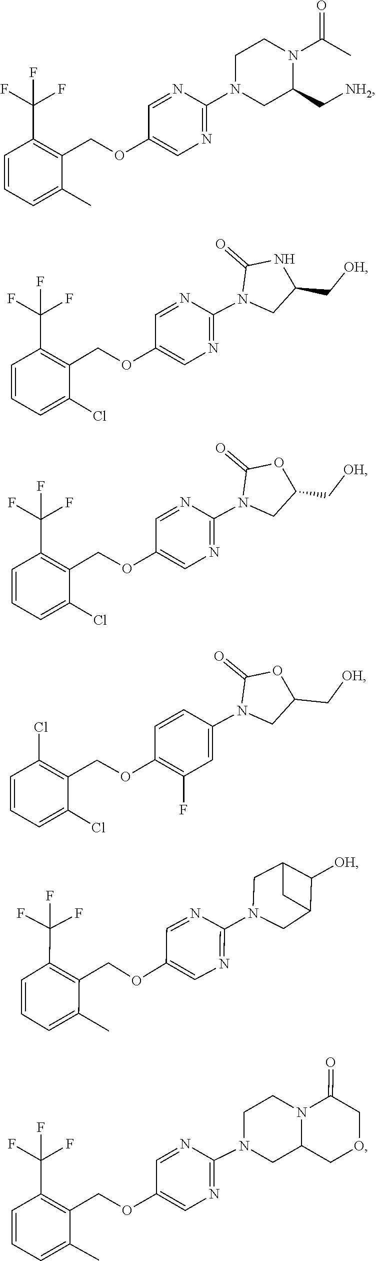 [SMILES: CC(=O)N1CCN(c2ncc(OCc3c(C)cccc3C(F)(F)F)cn2)C[C@@H]1CN ; O=C1N[C@@H](CO)CN1c1ncc(OCc2c(Cl)cccc2C(F)(F)F)cn1 ; O=C1O[C@H](CO)CN1c1ncc(OCc2c(Cl)cccc2C(F)(F)F)cn1 ; O=C1OC(CO)CN1c1ccc(OCc2c(Cl)cccc2Cl)c(F)c1 ; Cc1cccc(C(F)(F)F)c1COc1cnc(N2CC3CC(C2)C3O)nc1 ; Cc1cccc(C(F)(F)F)c1COc1cnc(N2CCN3C(=O)COCC3C2)nc1] 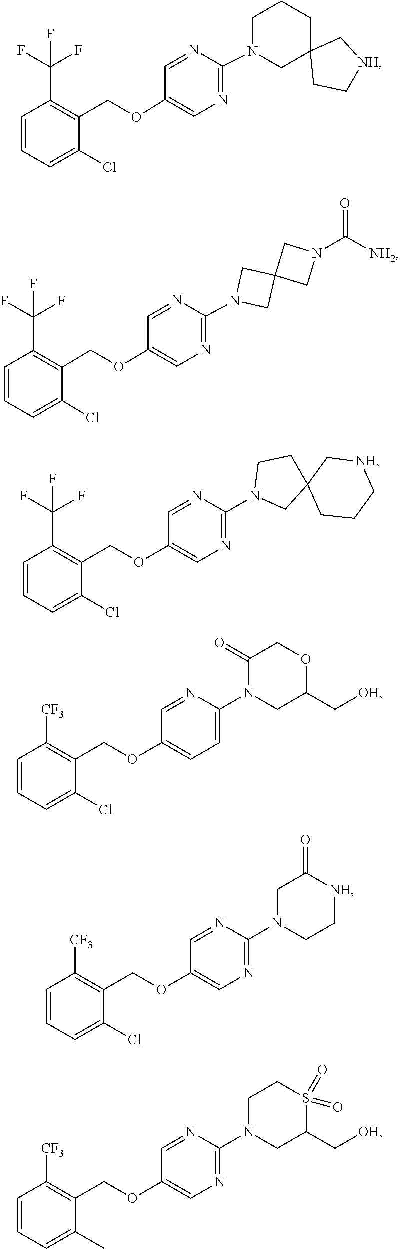 [SMILES: FC(F)(F)c1cccc(Cl)c1COc1cnc(N2CCCC3(CCNC3)C2)nc1 ; NC(=O)N1CC2(C1)CN(c1ncc(OCc3c(Cl)cccc3C(F)(F)F)cn1)C2 ; FC(F)(F)c1cccc(Cl)c1COc1cnc(N2CCC3(CCCNC3)C2)nc1 ; O=C1COC(CO)CN1c1ccc(OCc2c(Cl)cccc2C(F)(F)F)cn1 ; O=C1CN(c2ncc(OCc3c(Cl)cccc3C(F)(F)F)cn2)CCN1 ; Cc1cccc(C(F)(F)F)c1COc1cnc(N2CCS(=O)(=O)C(CO)C2)nc1] 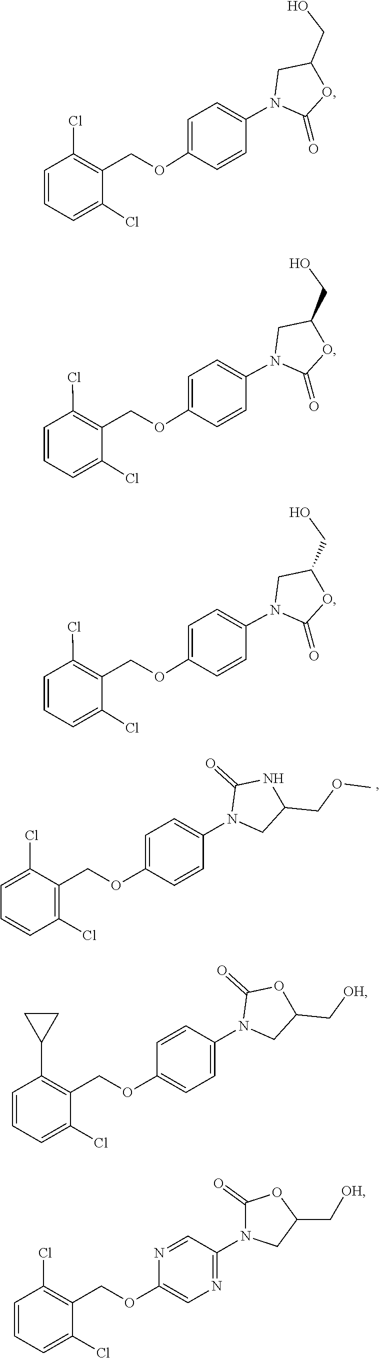 [SMILES: O=C1OC(CO)CN1c1ccc(OCc2c(Cl)cccc2Cl)cc1 ; O=C1O[C@H](CO)CN1c1ccc(OCc2c(Cl)cccc2Cl)cc1 ; O=C1O[C@@H](CO)CN1c1ccc(OCc2c(Cl)cccc2Cl)cc1 ; COCC1CN(c2ccc(OCc3c(Cl)cccc3Cl)cc2)C(=O)N1 ; O=C1OC(CO)CN1c1ccc(OCc2c(Cl)cccc2C2CC2)cc1 ; O=C1OC(CO)CN1c1cnc(OCc2c(Cl)cccc2Cl)cn1] 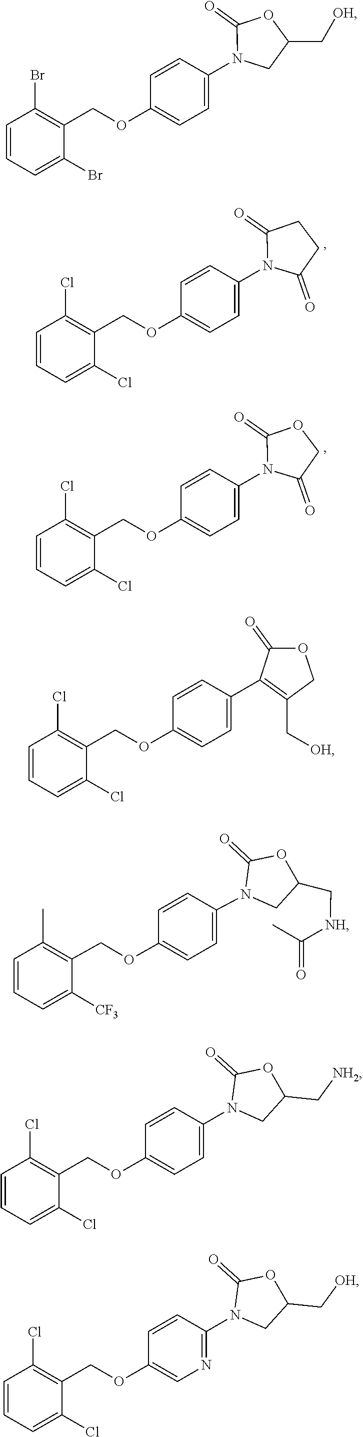 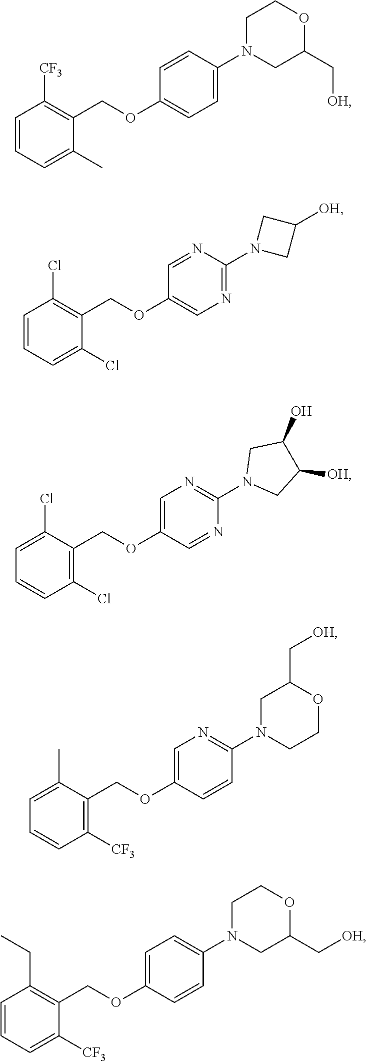 [SMILES: Cc1cccc(C(F)(F)F)c1COc1ccc(N2CCOC(CO)C2)cc1 ; OC1CN(c2ncc(OCc3c(Cl)cccc3Cl)cn2)C1 ; O[C@@H]1CN(c2ncc(OCc3c(Cl)cccc3Cl)cn2)C[C@@H]1O ; Cc1cccc(C(F)(F)F)c1COc1ccc(N2CCOC(CO)C2)nc1 ; CCc1cccc(C(F)(F)F)c1COc1ccc(N2CCOC(CO)C2)cc1] 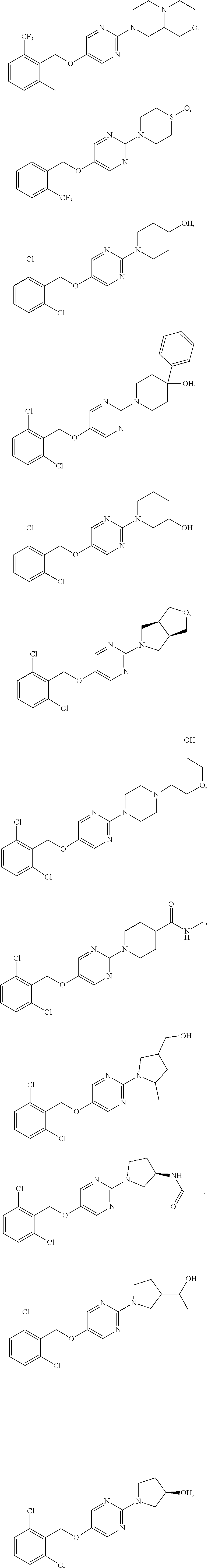 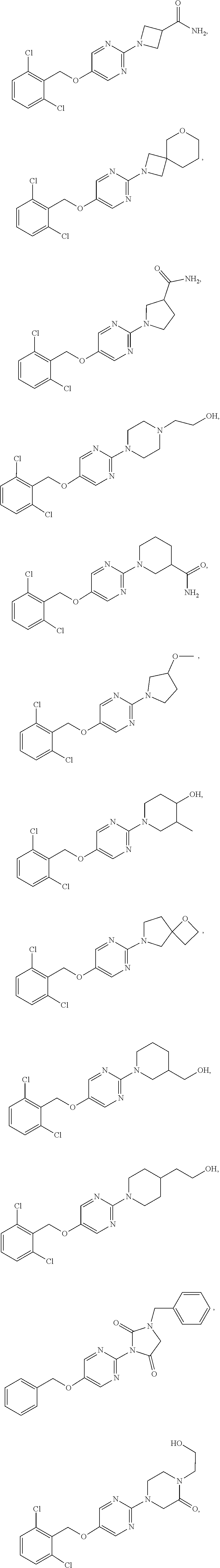 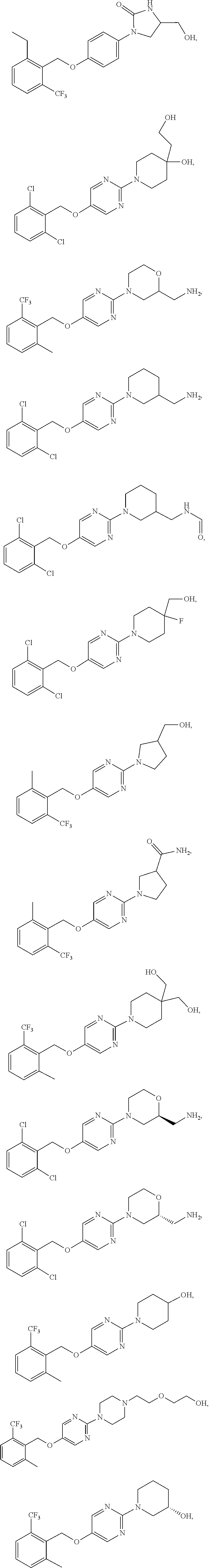 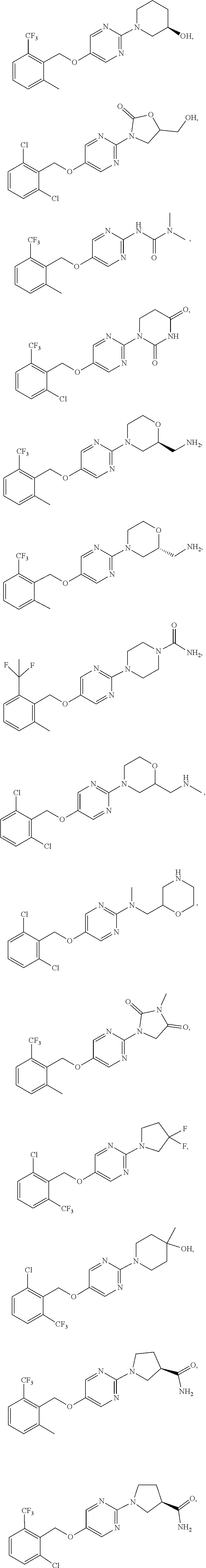 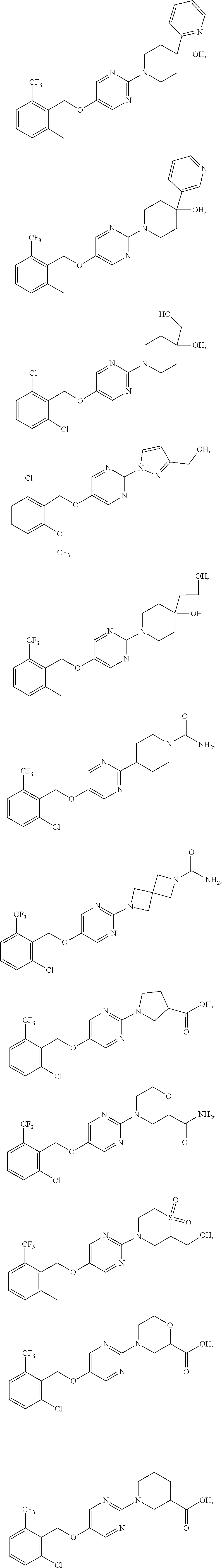 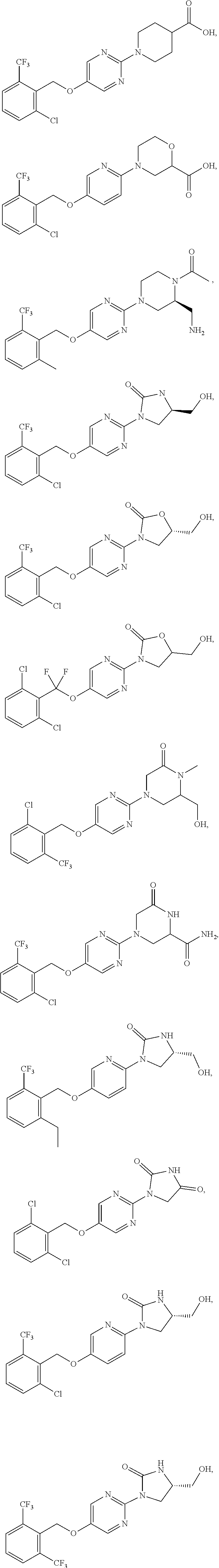 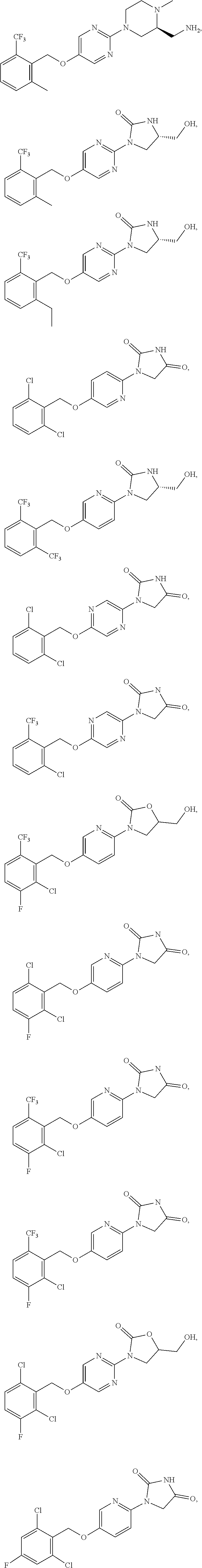 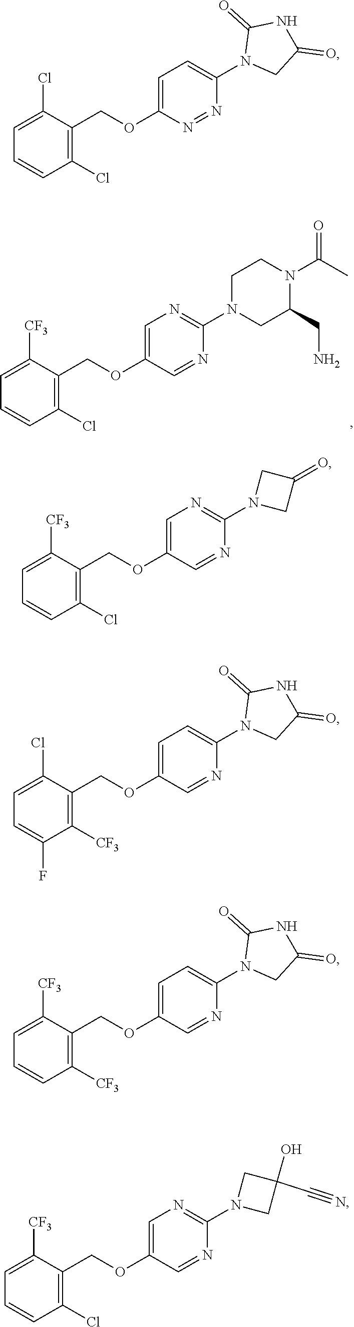 [SMILES: O=C1CN(c2ccc(OCc3c(Cl)cccc3Cl)nn2)C(=O)N1 ; CC(=O)N1CCN(c2ncc(OCc3c(Cl)cccc3C(F)(F)F)cn2)C[C@@H]1CN ; O=C1CN(c2ncc(OCc3c(Cl)cccc3C(F)(F)F)cn2)C1 ; O=C1CN(c2ccc(OCc3c(Cl)ccc(F)c3C(F)(F)F)cn2)C(=O)N1 ; O=C1CN(c2ccc(OCc3c(C(F)(F)F)cccc3C(F)(F)F)cn2)C(=O)N1 ; N#CC1(O)CN(c2ncc(OCc3c(Cl)cccc3C(F)(F)F)cn2)C1]
and pharmaceutically acceptable salts thereof.

25. (canceled)

26. The compound of Claim 1, wherein R⁶ is selected from the group consisting of:

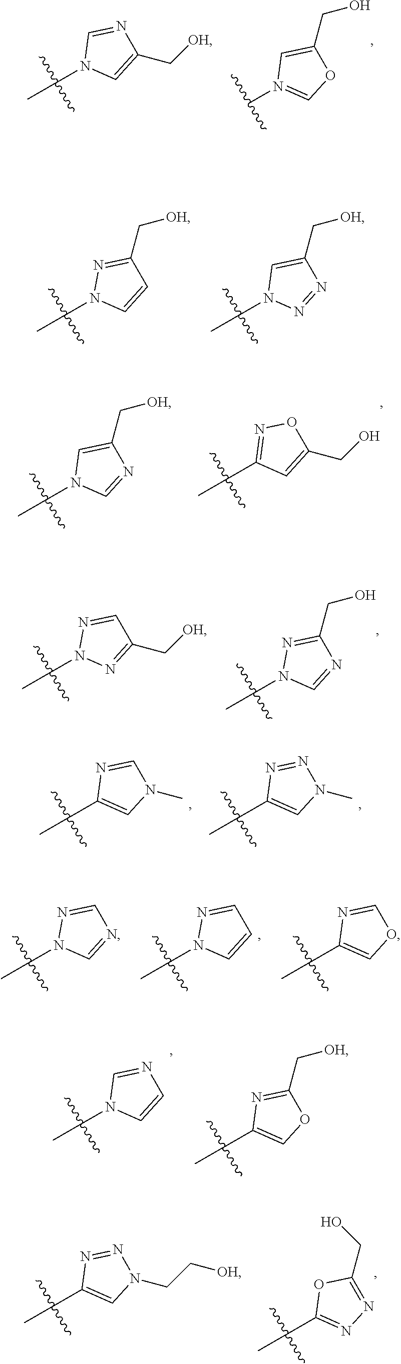

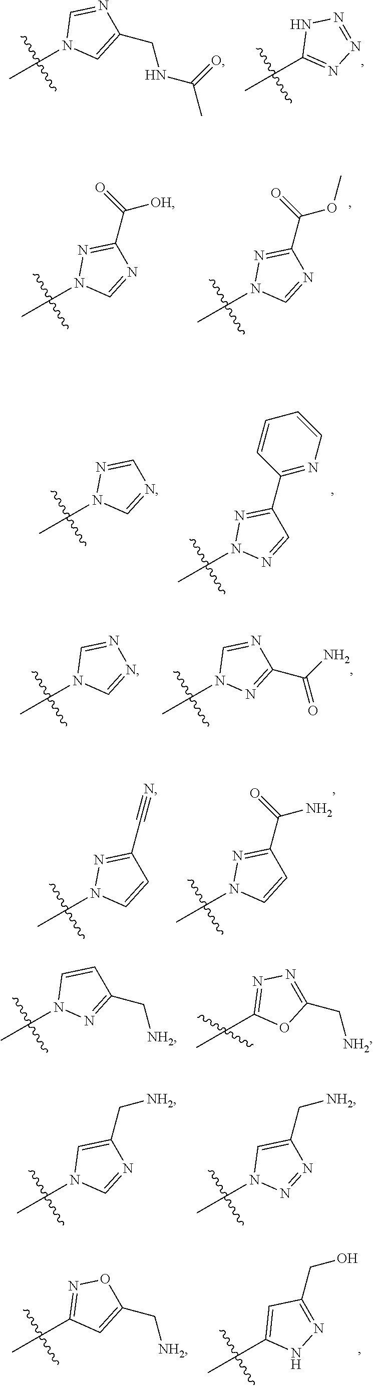

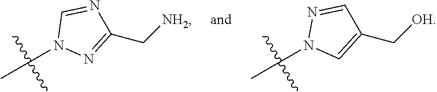 [SMILES: *n1cnc(CN)n1 ; *n1cc(CO)cn1]

27. A compound selected from the group consisting of: 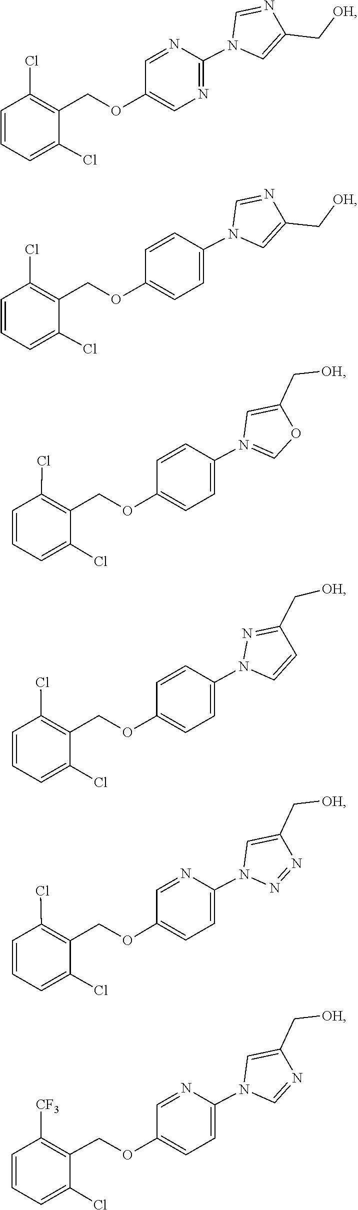 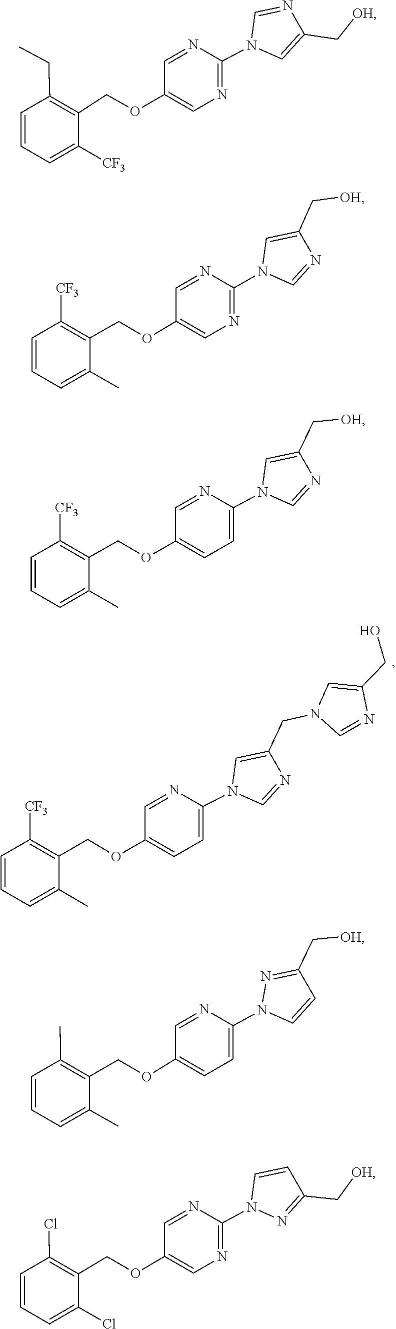 [SMILES: CCc1cccc(C(F)(F)F)c1COc1cnc(-n2cnc(CO)c2)nc1 ; Cc1cccc(C(F)(F)F)c1COc1cnc(-n2cnc(CO)c2)nc1 ; Cc1cccc(C(F)(F)F)c1COc1ccc(-n2cnc(CO)c2)nc1 ; Cc1cccc(C(F)(F)F)c1COc1ccc(-n2cnc(Cn3cnc(CO)c3)c2)nc1 ; Cc1cccc(C)c1COc1ccc(-n2ccc(CO)n2)nc1 ; OCc1ccn(-c2ncc(OCc3c(Cl)cccc3Cl)cn2)n1] 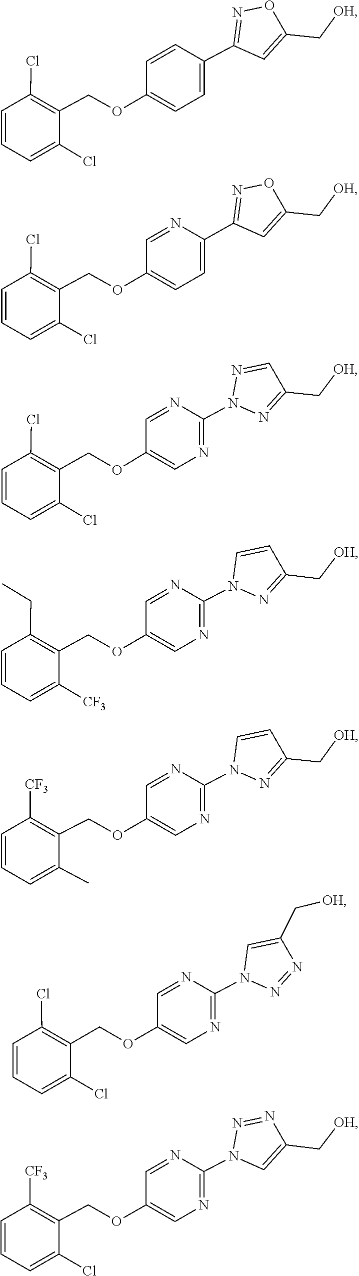 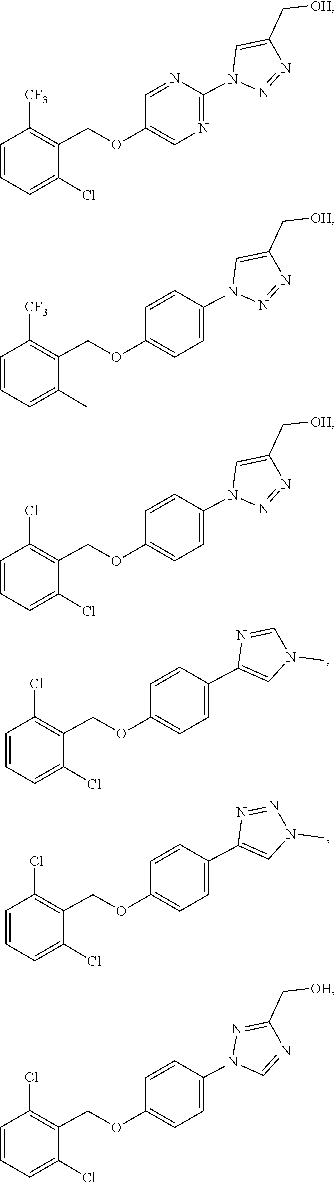 [SMILES: OCc1cn(-c2ncc(OCc3c(Cl)cccc3C(F)(F)F)cn2)nn1 ; Cc1cccc(C(F)(F)F)c1COc1ccc(-n2cc(CO)nn2)cc1 ; OCc1cn(-c2ccc(OCc3c(Cl)cccc3Cl)cc2)nn1 ; Cn1cnc(-c2ccc(OCc3c(Cl)cccc3Cl)cc2)c1 ; Cn1cc(-c2ccc(OCc3c(Cl)cccc3Cl)cc2)nn1 ; OCc1ncn(-c2ccc(OCc3c(Cl)cccc3Cl)cc2)n1] 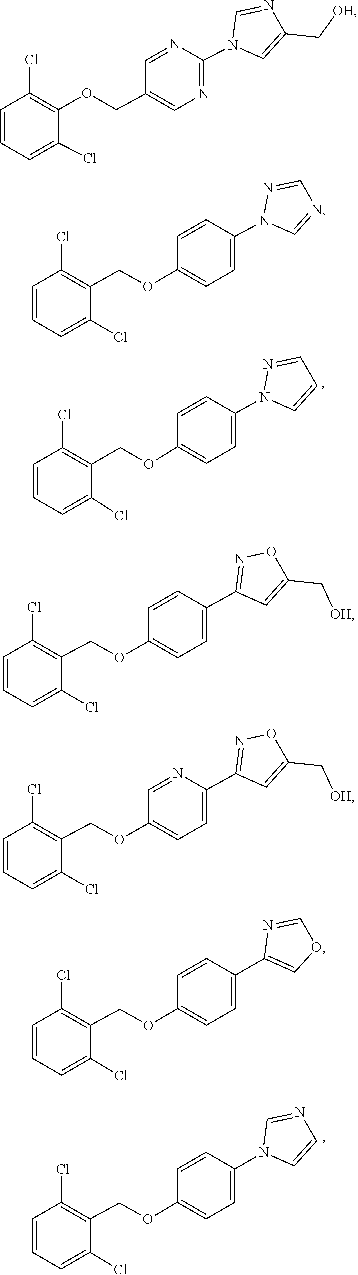 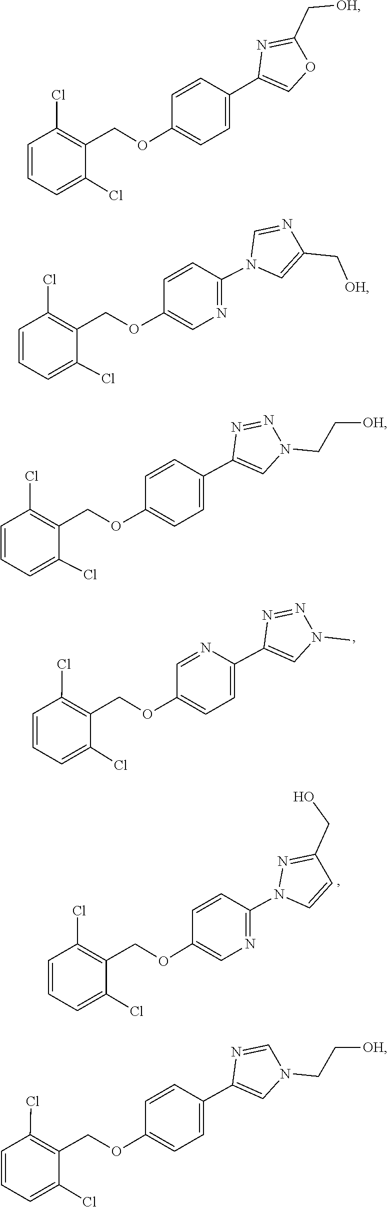 [SMILES: OCc1nc(-c2ccc(OCc3c(Cl)cccc3Cl)cc2)co1 ; OCc1cn(-c2ccc(OCc3c(Cl)cccc3Cl)cn2)cn1 ; OCCn1cc(-c2ccc(OCc3c(Cl)cccc3Cl)cc2)nn1 ; Cn1cc(-c2ccc(OCc3c(Cl)cccc3Cl)cn2)nn1 ; OCc1ccn(-c2ccc(OCc3c(Cl)cccc3Cl)cn2)n1 ; OCCn1cnc(-c2ccc(OCc3c(Cl)cccc3Cl)cc2)c1] 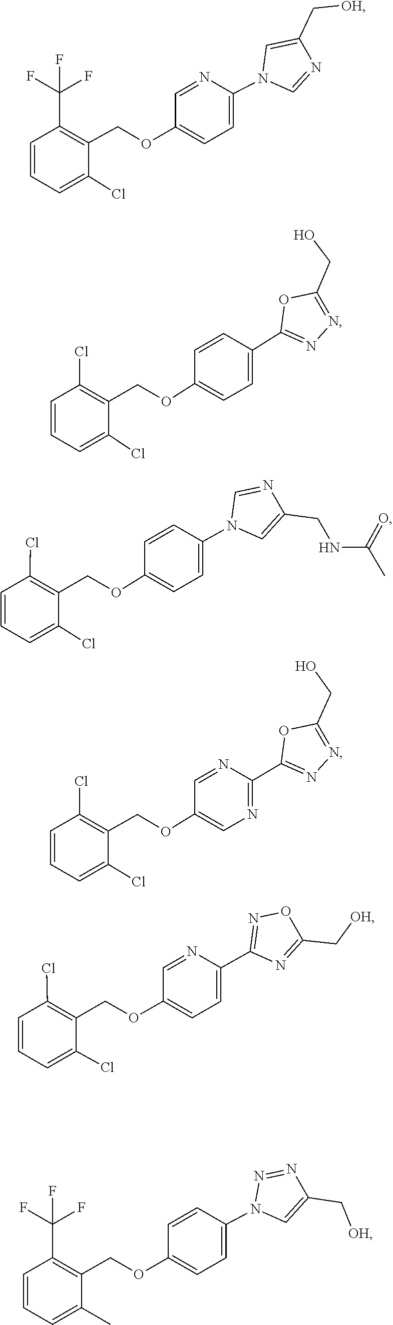 [SMILES: OCc1cn(-c2ccc(OCc3c(Cl)cccc3C(F)(F)F)cn2)cn1 ; OCc1nnc(-c2ccc(OCc3c(Cl)cccc3Cl)cc2)o1 ; CC(=O)NCc1cn(-c2ccc(OCc3c(Cl)cccc3Cl)cc2)cn1 ; OCc1nnc(-c2ncc(OCc3c(Cl)cccc3Cl)cn2)o1 ; OCc1nc(-c2ccc(OCc3c(Cl)cccc3Cl)cn2)no1 ; Cc1cccc(C(F)(F)F)c1COc1ccc(-n2cc(CO)nn2)cc1] 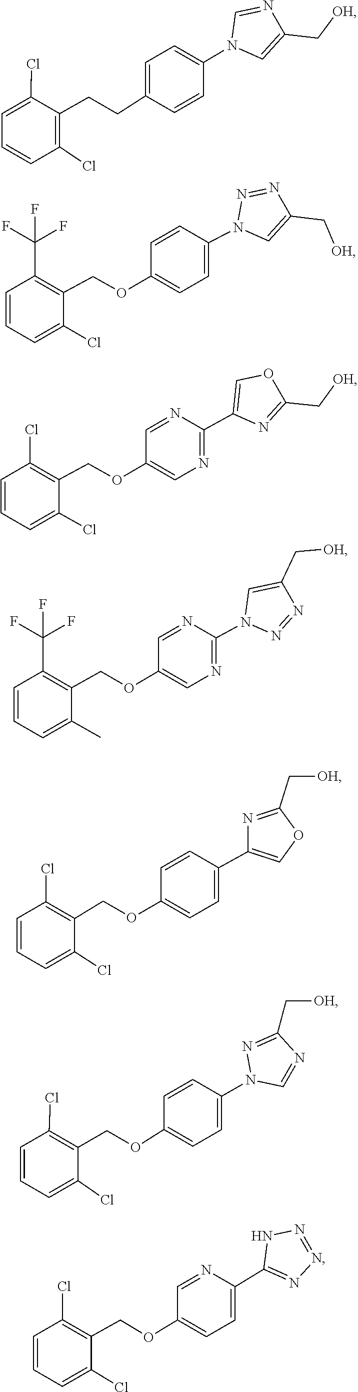 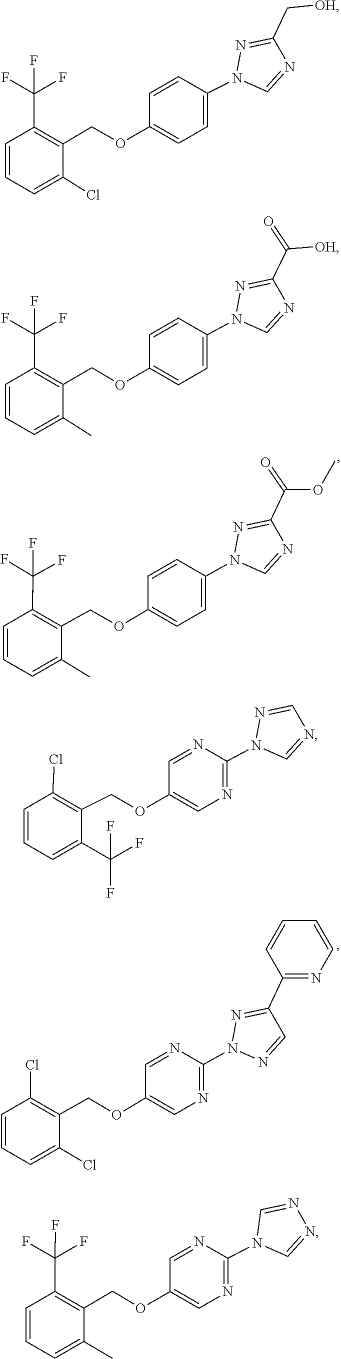 [SMILES: OCc1ncn(-c2ccc(OCc3c(Cl)cccc3C(F)(F)F)cc2)n1 ; Cc1cccc(C(F)(F)F)c1COc1ccc(-n2cnc(C(=O)O)n2)cc1 ; COC(=O)c1ncn(-c2ccc(OCc3c(C)cccc3C(F)(F)F)cc2)n1 ; FC(F)(F)c1cccc(Cl)c1COc1cnc(-n2cncn2)nc1 ; Clc1cccc(Cl)c1COc1cnc(-n2ncc(-c3ccccn3)n2)nc1 ; Cc1cccc(C(F)(F)F)c1COc1cnc(-n2cnnc2)nc1] 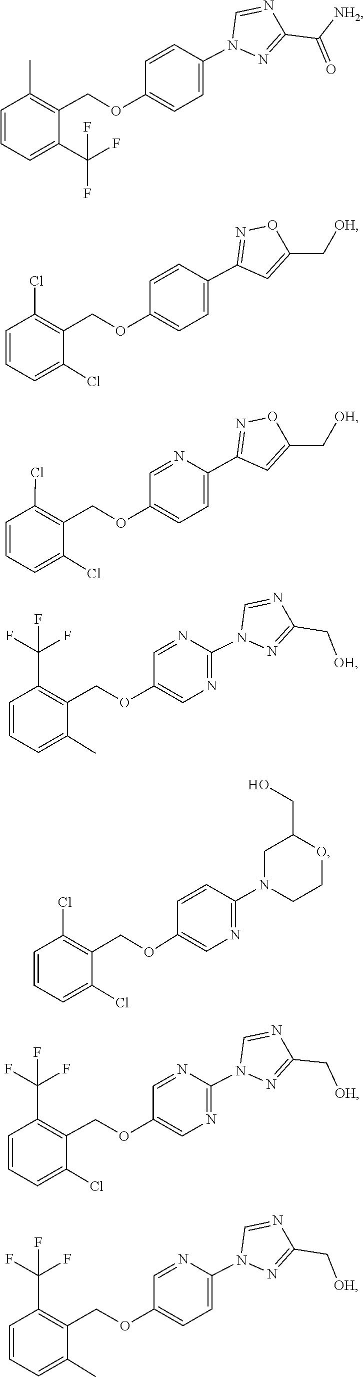 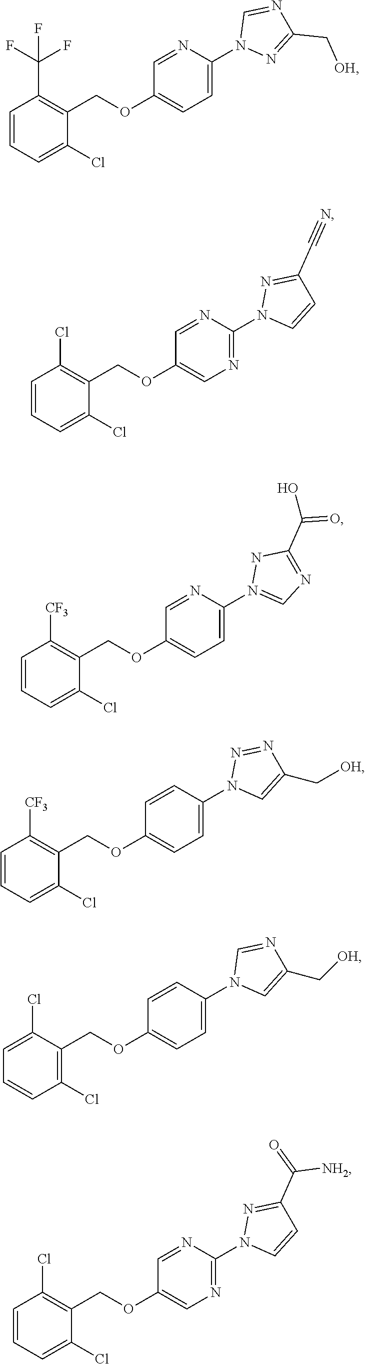 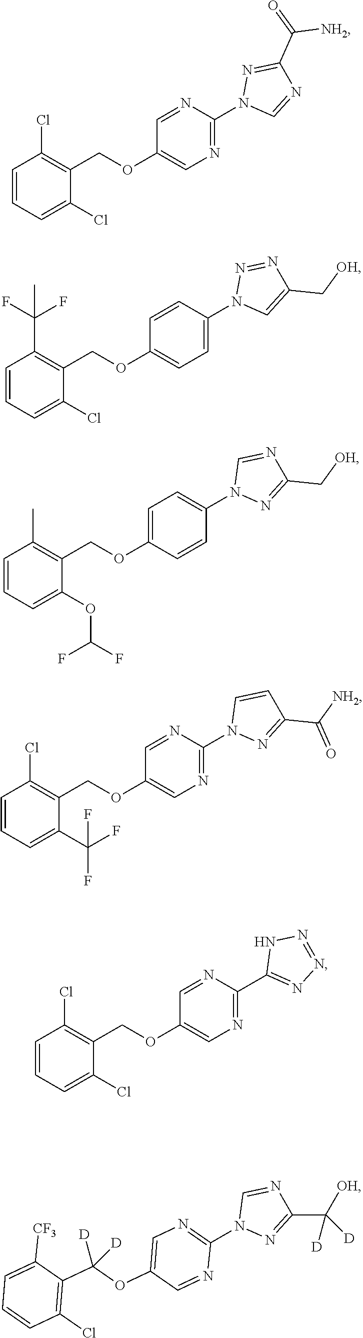 [SMILES: NC(=O)c1ncn(-c2ncc(OCc3c(Cl)cccc3Cl)cn2)n1 ; CC(F)(F)c1cccc(Cl)c1COc1ccc(-n2cc(CO)nn2)cc1 ; Cc1cccc(OC(F)F)c1COc1ccc(-n2cnc(CO)n2)cc1 ; NC(=O)c1ccn(-c2ncc(OCc3c(Cl)cccc3C(F)(F)F)cn2)n1 ; Clc1cccc(Cl)c1COc1cnc(-c2nnn[nH]2)nc1 ; [2H]C([2H])(O)c1ncn(-c2ncc(OC([2H])([2H])c3c(Cl)cccc3C(F)(F)F)cn2)n1] 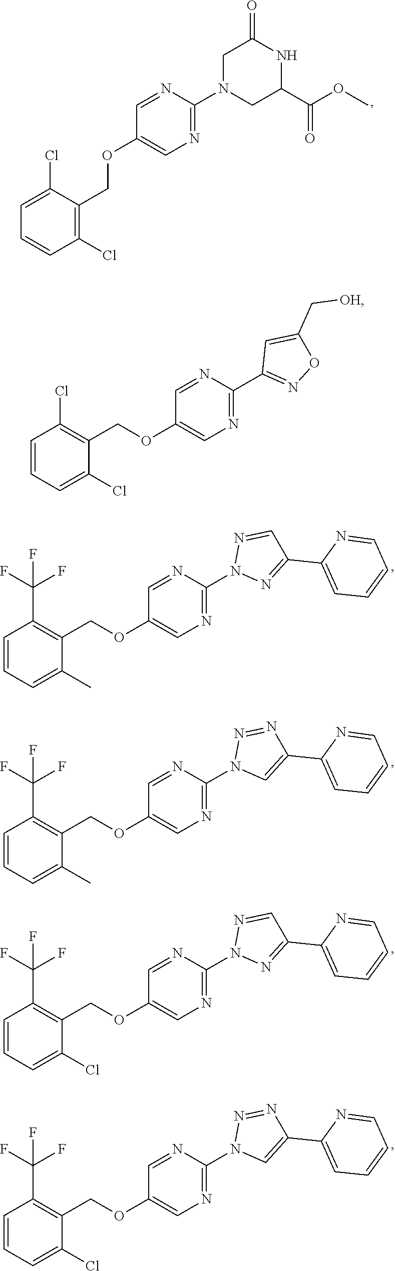 [SMILES: COC(=O)C1CN(c2ncc(OCc3c(Cl)cccc3Cl)cn2)CC(=O)N1 ; OCc1cc(-c2ncc(OCc3c(Cl)cccc3Cl)cn2)no1 ; Cc1cccc(C(F)(F)F)c1COc1cnc(-n2ncc(-c3ccccn3)n2)nc1 ; Cc1cccc(C(F)(F)F)c1COc1cnc(-n2cc(-c3ccccn3)nn2)nc1 ; FC(F)(F)c1cccc(Cl)c1COc1cnc(-n2ncc(-c3ccccn3)n2)nc1 ; FC(F)(F)c1cccc(Cl)c1COc1cnc(-n2cc(-c3ccccn3)nn2)nc1] 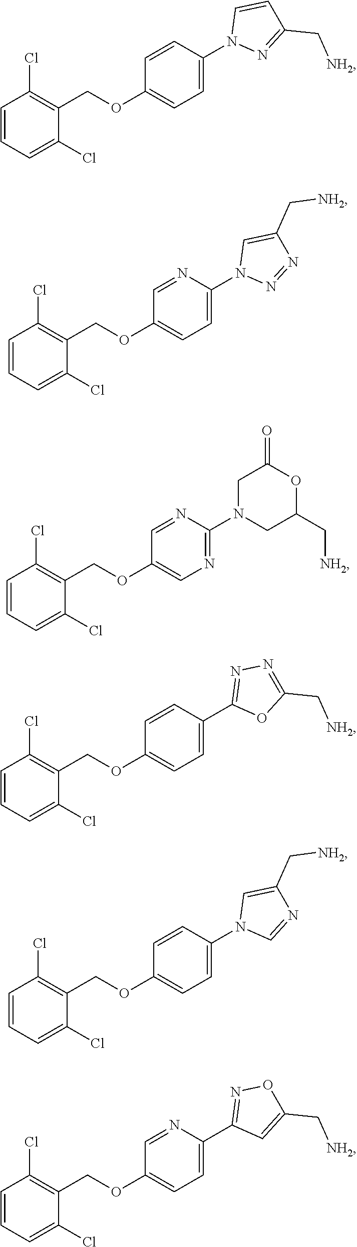 [SMILES: NCc1ccn(-c2ccc(OCc3c(Cl)cccc3Cl)cc2)n1 ; NCc1cn(-c2ccc(OCc3c(Cl)cccc3Cl)cn2)nn1 ; NCC1CN(c2ncc(OCc3c(Cl)cccc3Cl)cn2)CC(=O)O1 ; NCc1nnc(-c2ccc(OCc3c(Cl)cccc3Cl)cc2)o1 ; NCc1cn(-c2ccc(OCc3c(Cl)cccc3Cl)cc2)cn1 ; NCc1cc(-c2ccc(OCc3c(Cl)cccc3Cl)cn2)no1] 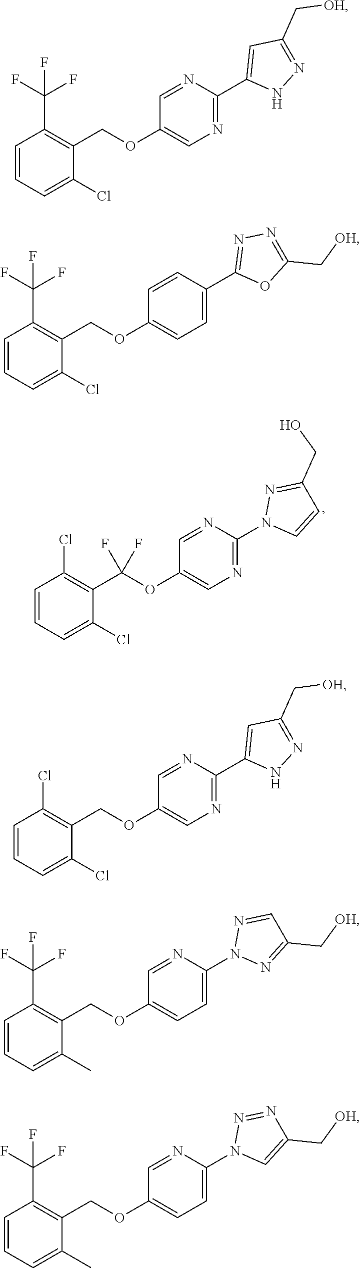 [SMILES: OCc1cc(-c2ncc(OCc3c(Cl)cccc3C(F)(F)F)cn2)[nH]n1 ; OCc1nnc(-c2ccc(OCc3c(Cl)cccc3C(F)(F)F)cc2)o1 ; OCc1ccn(-c2ncc(OC(F)(F)c3c(Cl)cccc3Cl)cn2)n1 ; OCc1cc(-c2ncc(OCc3c(Cl)cccc3Cl)cn2)[nH]n1 ; Cc1cccc(C(F)(F)F)c1COc1ccc(-n2ncc(CO)n2)nc1 ; Cc1cccc(C(F)(F)F)c1COc1ccc(-n2cc(CO)nn2)nc1] 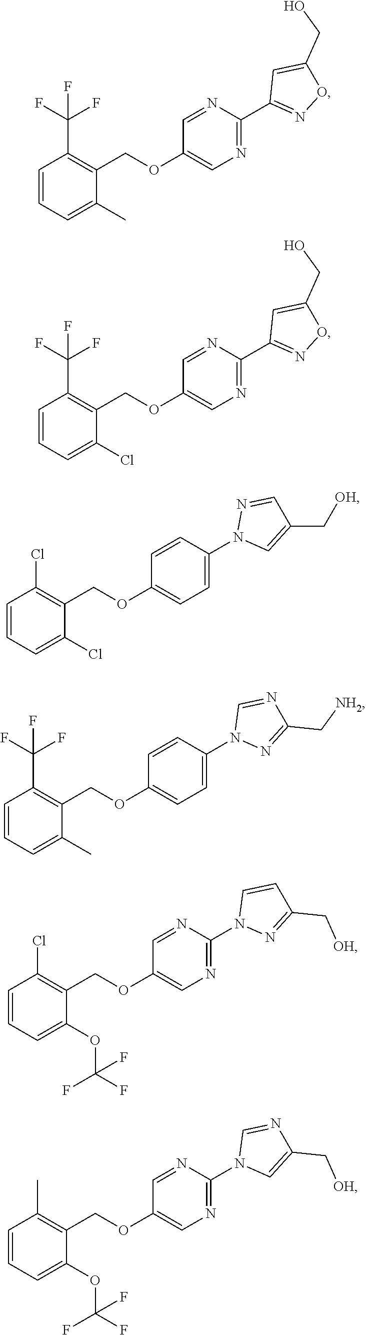 [SMILES: Cc1cccc(C(F)(F)F)c1COc1cnc(-c2cc(CO)on2)nc1 ; OCc1cc(-c2ncc(OCc3c(Cl)cccc3C(F)(F)F)cn2)no1 ; OCc1cnn(-c2ccc(OCc3c(Cl)cccc3Cl)cc2)c1 ; Cc1cccc(C(F)(F)F)c1COc1ccc(-n2cnc(CN)n2)cc1 ; OCc1ccn(-c2ncc(OCc3c(Cl)cccc3OC(F)(F)F)cn2)n1 ; Cc1cccc(OC(F)(F)F)c1COc1cnc(-n2cnc(CO)c2)nc1] 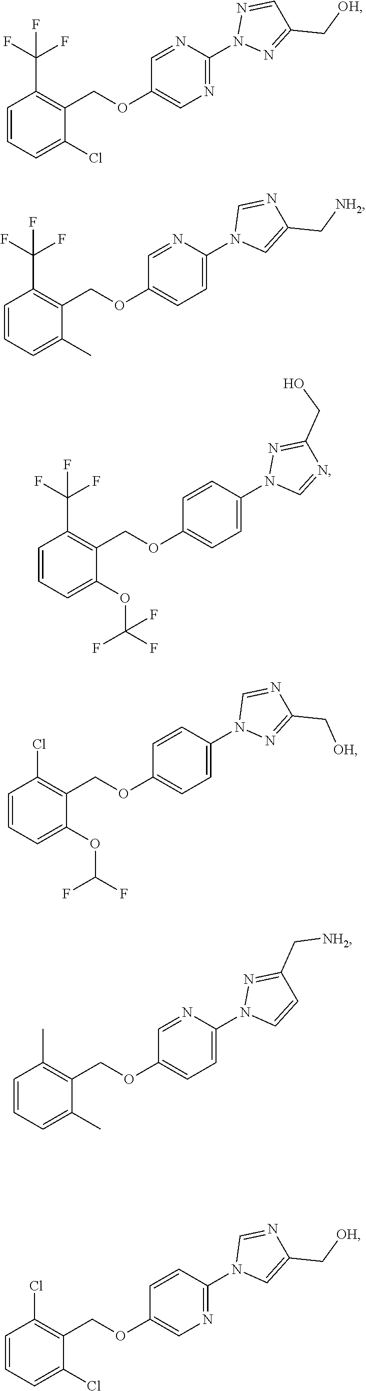 [SMILES: OCc1cnn(-c2ncc(OCc3c(Cl)cccc3C(F)(F)F)cn2)n1 ; Cc1cccc(C(F)(F)F)c1COc1ccc(-n2cnc(CN)c2)nc1 ; OCc1ncn(-c2ccc(OCc3c(OC(F)(F)F)cccc3C(F)(F)F)cc2)n1 ; OCc1ncn(-c2ccc(OCc3c(Cl)cccc3OC(F)F)cc2)n1 ; Cc1cccc(C)c1COc1ccc(-n2ccc(CN)n2)nc1 ; OCc1cn(-c2ccc(OCc3c(Cl)cccc3Cl)cn2)cn1] 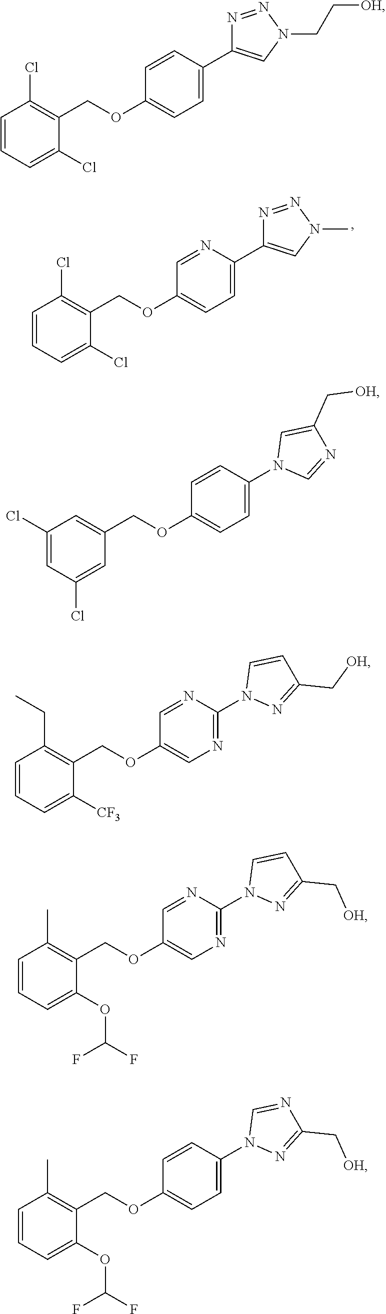 [SMILES: OCCn1cc(-c2ccc(OCc3c(Cl)cccc3Cl)cc2)nn1 ; Cn1cc(-c2ccc(OCc3c(Cl)cccc3Cl)cn2)nn1 ; OCc1cn(-c2ccc(OCc3cc(Cl)cc(Cl)c3)cc2)cn1 ; CCc1cccc(C(F)(F)F)c1COc1cnc(-n2ccc(CO)n2)nc1 ; Cc1cccc(OC(F)F)c1COc1cnc(-n2ccc(CO)n2)nc1 ; Cc1cccc(OC(F)F)c1COc1ccc(-n2cnc(CO)n2)cc1] 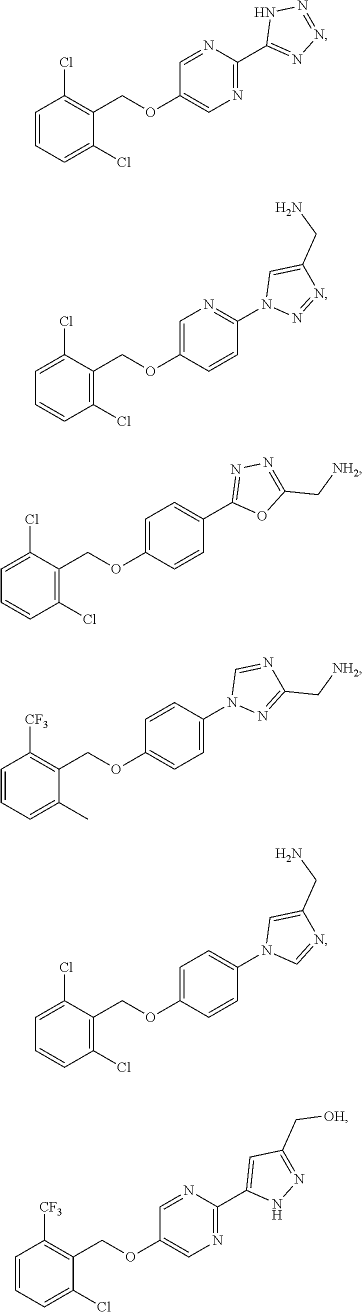 [SMILES: Clc1cccc(Cl)c1COc1cnc(-c2nnn[nH]2)nc1 ; NCc1cn(-c2ccc(OCc3c(Cl)cccc3Cl)cn2)nn1 ; NCc1nnc(-c2ccc(OCc3c(Cl)cccc3Cl)cc2)o1 ; Cc1cccc(C(F)(F)F)c1COc1ccc(-n2cnc(CN)n2)cc1 ; NCc1cn(-c2ccc(OCc3c(Cl)cccc3Cl)cc2)cn1 ; OCc1cc(-c2ncc(OCc3c(Cl)cccc3C(F)(F)F)cn2)[nH]n1] 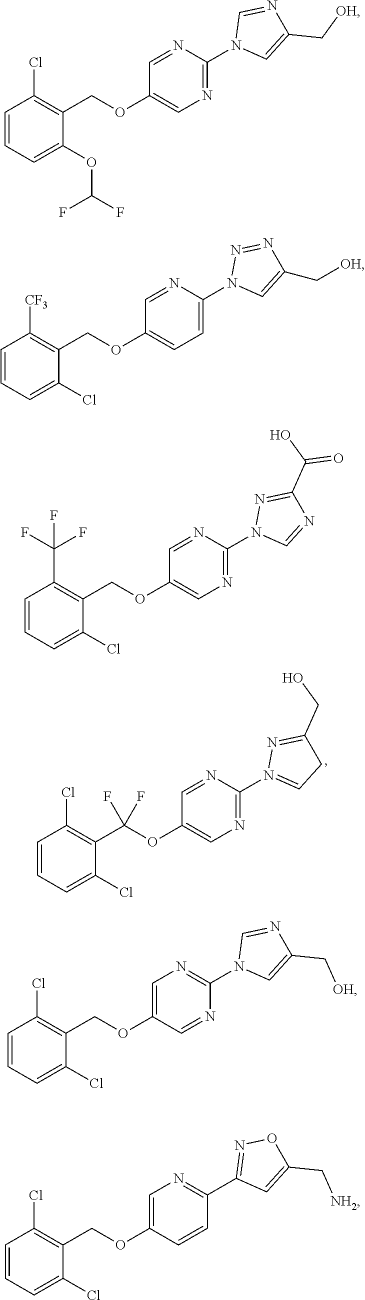 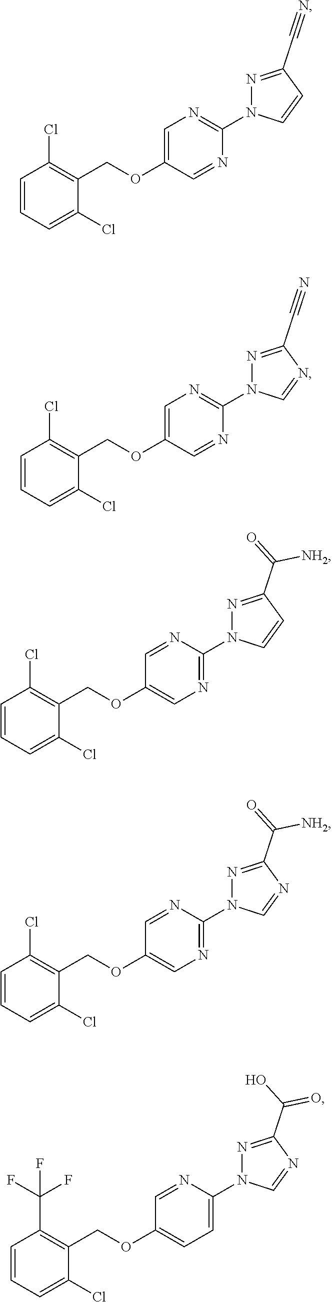 [SMILES: N#Cc1ccn(-c2ncc(OCc3c(Cl)cccc3Cl)cn2)n1 ; N#Cc1ncn(-c2ncc(OCc3c(Cl)cccc3Cl)cn2)n1 ; NC(=O)c1ccn(-c2ncc(OCc3c(Cl)cccc3Cl)cn2)n1 ; NC(=O)c1ncn(-c2ncc(OCc3c(Cl)cccc3Cl)cn2)n1 ; O=C(O)c1ncn(-c2ccc(OCc3c(Cl)cccc3C(F)(F)F)cn2)n1] 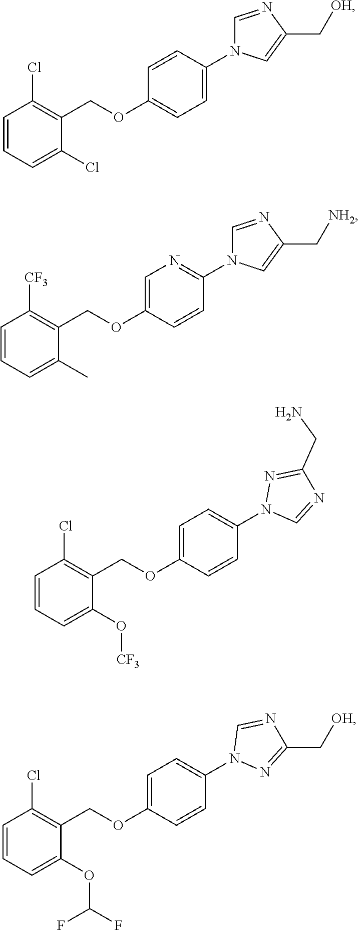 [SMILES: OCc1cn(-c2ccc(OCc3c(Cl)cccc3Cl)cc2)cn1 ; Cc1cccc(C(F)(F)F)c1COc1ccc(-n2cnc(CN)c2)nc1 ; NCc1ncn(-c2ccc(OCc3c(Cl)cccc3OC(F)(F)F)cc2)n1 ; OCc1ncn(-c2ccc(OCc3c(Cl)cccc3OC(F)F)cc2)n1] 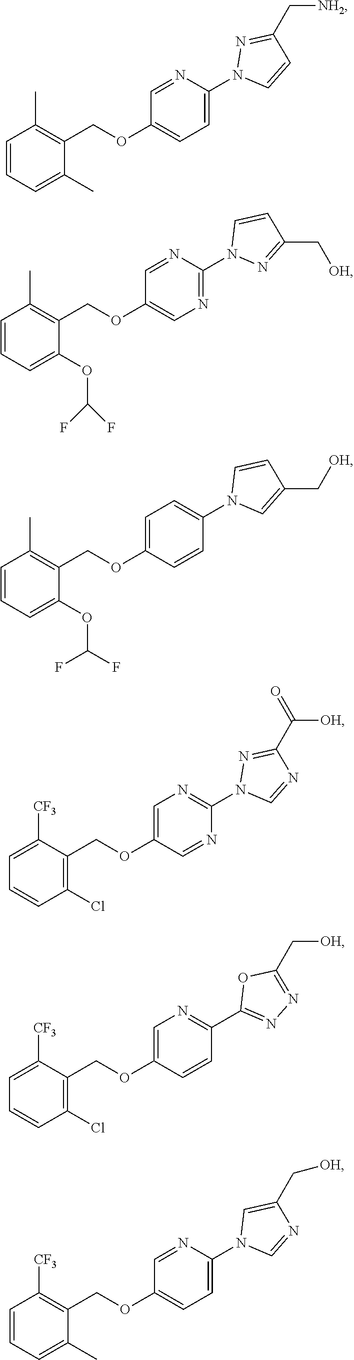 [SMILES: Cc1cccc(C)c1COc1ccc(-n2ccc(CN)n2)nc1 ; Cc1cccc(OC(F)F)c1COc1cnc(-n2ccc(CO)n2)nc1 ; Cc1cccc(OC(F)F)c1COc1ccc(-n2ccc(CO)c2)cc1 ; O=C(O)c1ncn(-c2ncc(OCc3c(Cl)cccc3C(F)(F)F)cn2)n1 ; OCc1nnc(-c2ccc(OCc3c(Cl)cccc3C(F)(F)F)cn2)o1 ; Cc1cccc(C(F)(F)F)c1COc1ccc(-n2cnc(CO)c2)nc1] 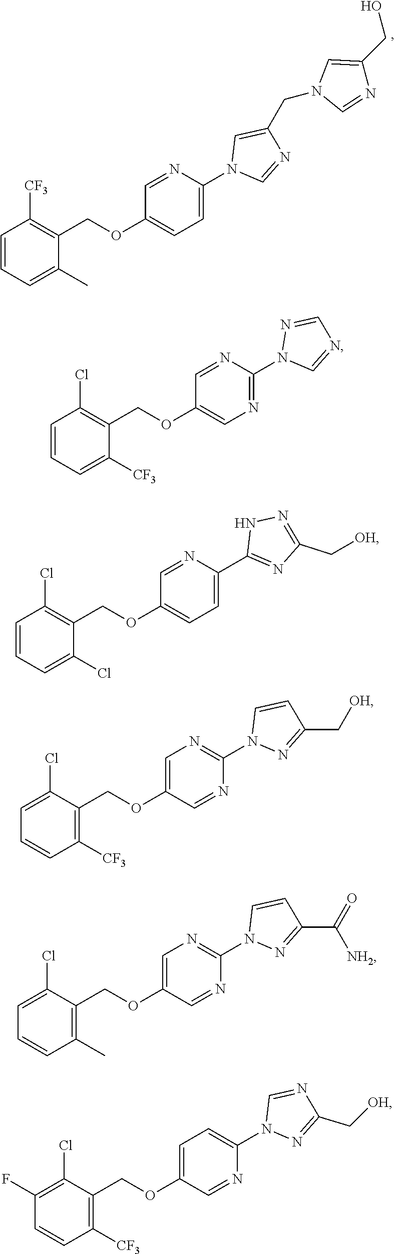 [SMILES: Cc1cccc(C(F)(F)F)c1COc1ccc(-n2cnc(Cn3cnc(CO)c3)c2)nc1 ; FC(F)(F)c1cccc(Cl)c1COc1cnc(-n2cncn2)nc1 ; OCc1n[nH]c(-c2ccc(OCc3c(Cl)cccc3Cl)cn2)n1 ; OCc1ccn(-c2ncc(OCc3c(Cl)cccc3C(F)(F)F)cn2)n1 ; Cc1cccc(Cl)c1COc1cnc(-n2ccc(C(N)=O)n2)nc1 ; OCc1ncn(-c2ccc(OCc3c(C(F)(F)F)ccc(F)c3Cl)cn2)n1] 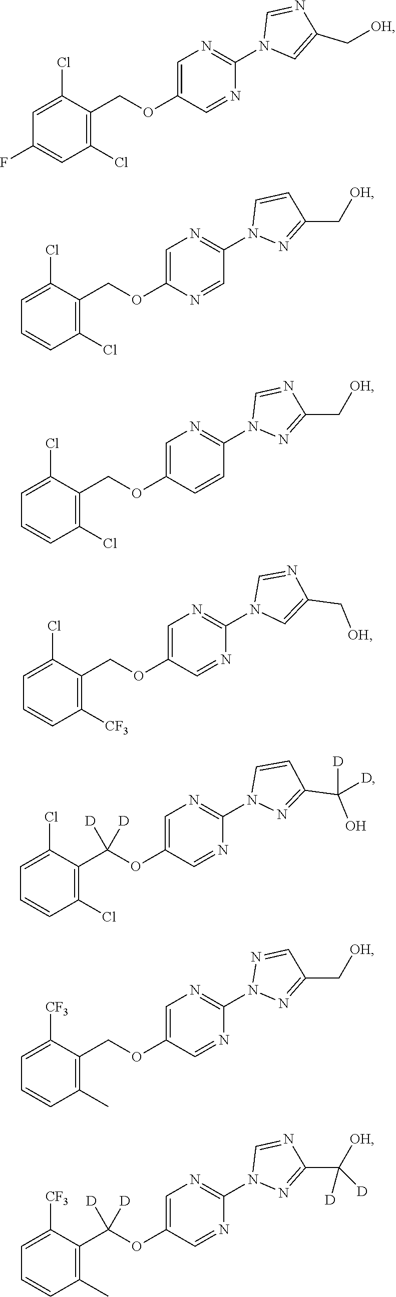 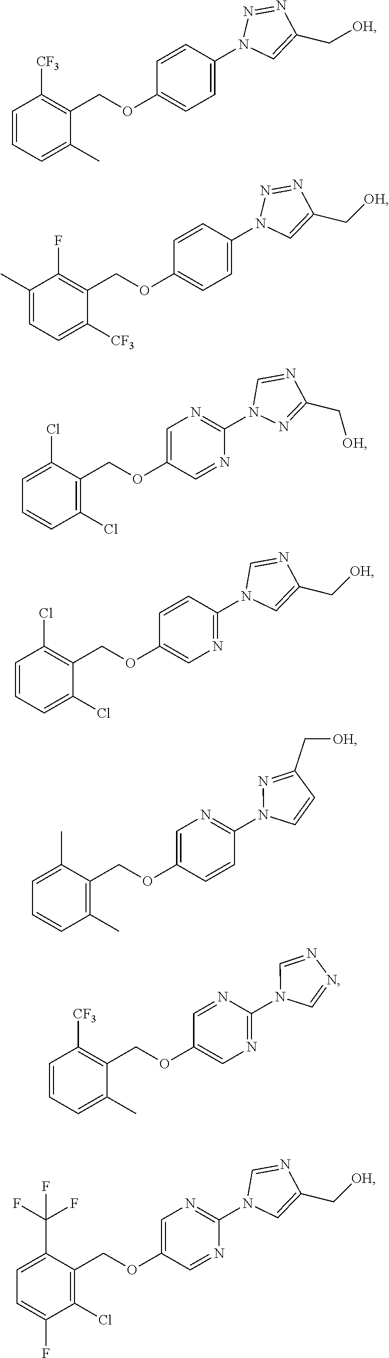 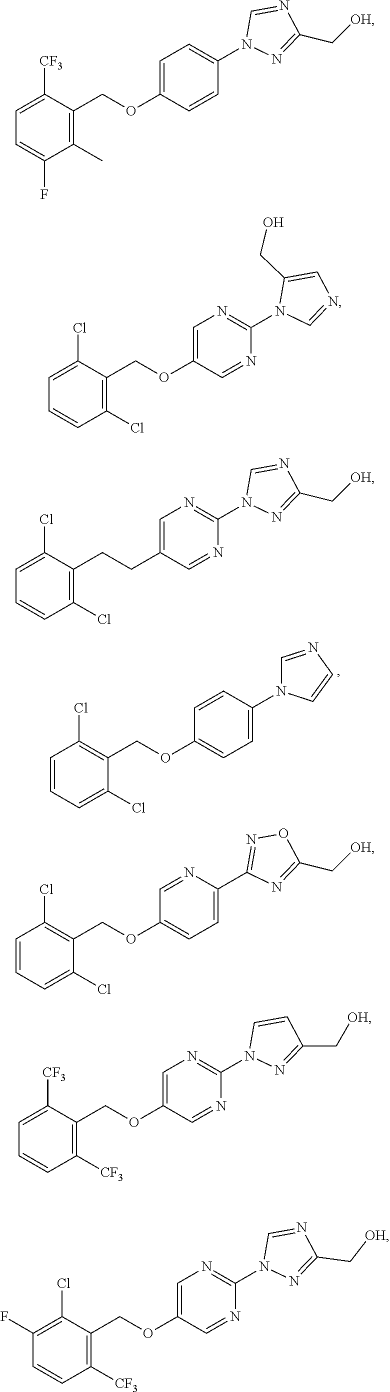 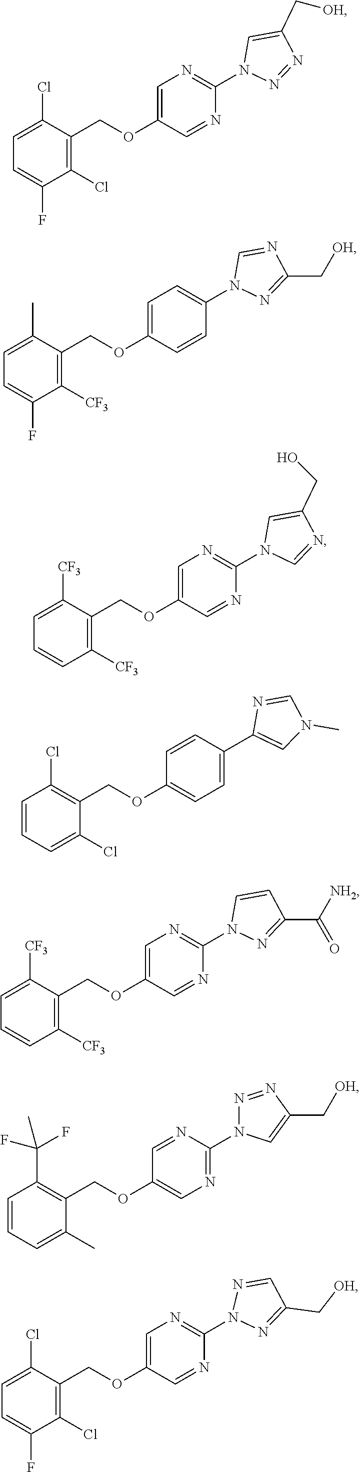 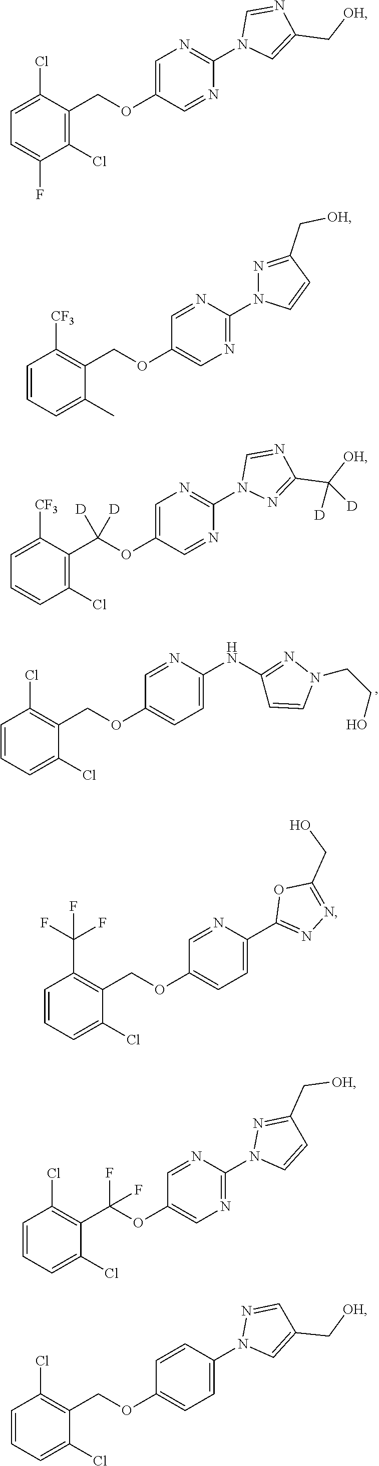 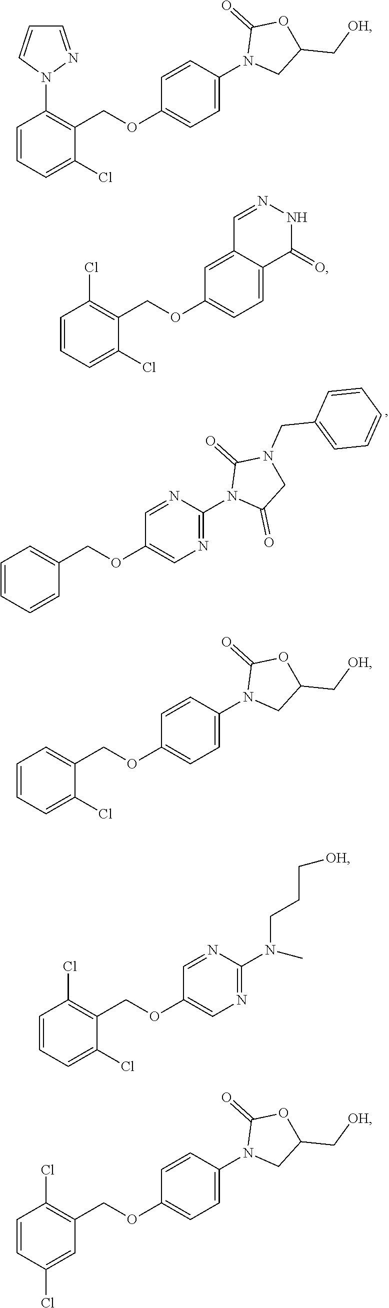 [SMILES: O=C1OC(CO)CN1c1ccc(OCc2c(Cl)cccc2-n2cccn2)cc1 ; O=c1[nH]ncc2cc(OCc3c(Cl)cccc3Cl)ccc12 ; O=C1CN(Cc2ccccc2)C(=O)N1c1ncc(OCc2ccccc2)cn1 ; O=C1OC(CO)CN1c1ccc(OCc2ccccc2Cl)cc1 ; CN(CCCO)c1ncc(OCc2c(Cl)cccc2Cl)cn1 ; O=C1OC(CO)CN1c1ccc(OCc2cc(Cl)ccc2Cl)cc1] 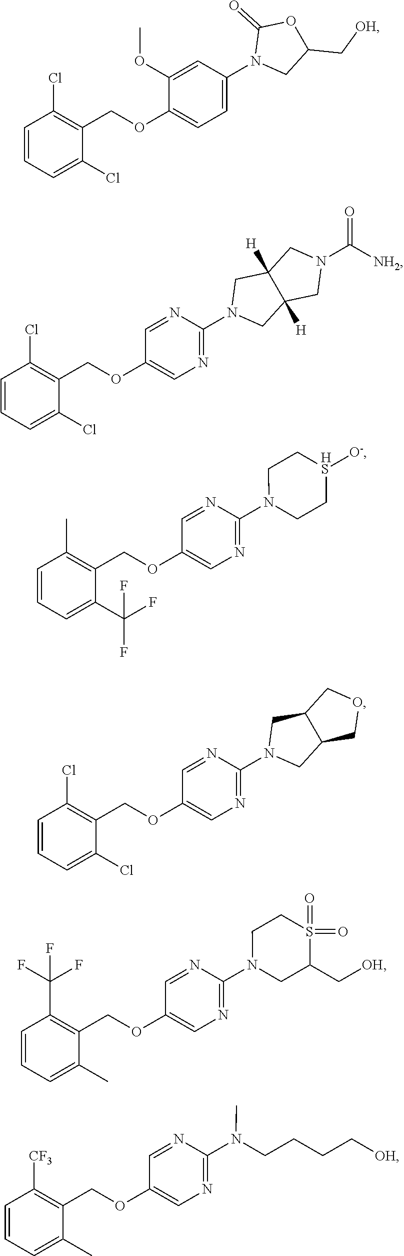 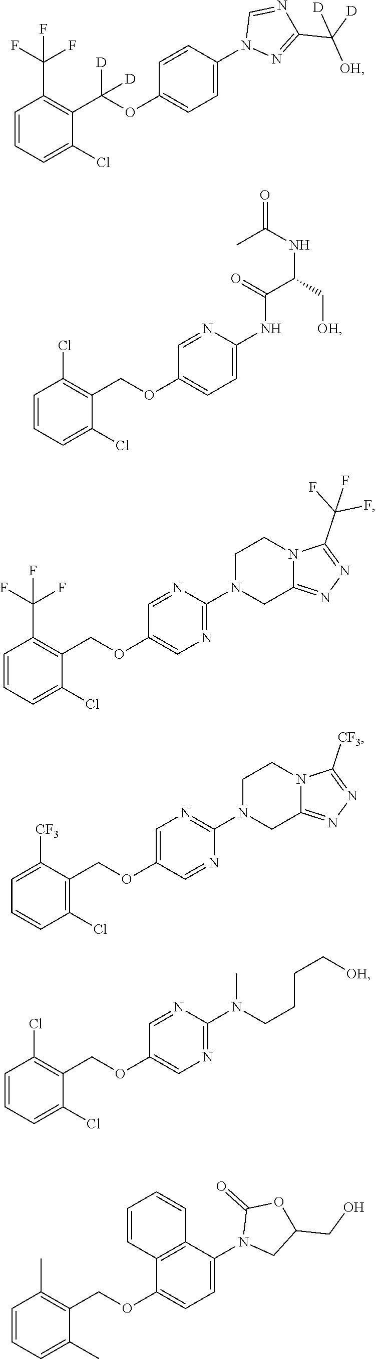 [SMILES: [2H]C([2H])(O)c1ncn(-c2ccc(OC([2H])([2H])c3c(Cl)cccc3C(F)(F)F)cc2)n1 ; CC(=O)N[C@H](CO)C(=O)Nc1ccc(OCc2c(Cl)cccc2Cl)cn1 ; FC(F)(F)c1cccc(Cl)c1COc1cnc(N2CCn3c(nnc3C(F)(F)F)C2)nc1 ; FC(F)(F)c1cccc(Cl)c1COc1cnc(N2CCn3c(nnc3C(F)(F)F)C2)nc1 ; CN(CCCCO)c1ncc(OCc2c(Cl)cccc2Cl)cn1 ; Cc1cccc(C)c1COc1ccc(N2CC(CO)OC2=O)c2ccccc12] 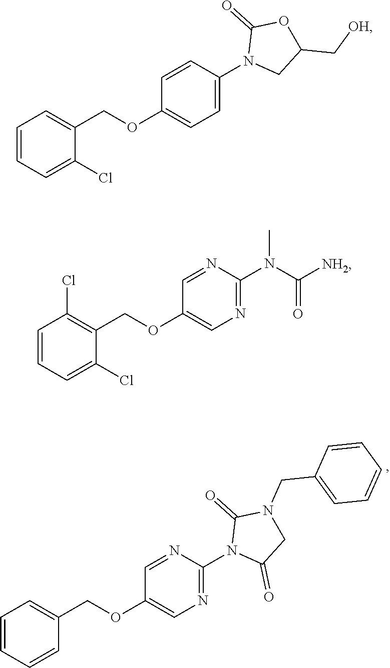 [SMILES: O=C1OC(CO)CN1c1ccc(OCc2ccccc2Cl)cc1 ; CN(C(N)=O)c1ncc(OCc2c(Cl)cccc2Cl)cn1 ; O=C1CN(Cc2ccccc2)C(=O)N1c1ncc(OCc2ccccc2)cn1]
and pharmaceutically acceptable salts thereof.

28. (canceled)

29. A pharmaceutical composition comprising a therapeutically effective amount of the compounds of Claim 1 and a pharmaceutically acceptable excipient.

30. The pharmaceutical composition of claim 29, further comprising an aminoglycoside, an eRF3 modulator, or a combination thereof.

31. (canceled)

32. (canceled)

33. (canceled)

34. A method of enhancing readthrough of genes containing premature termination codons, the method comprising administering to a subject in need thereof: (i) a readthrough modulator; and (ii) an aminoglycoside, wherein the readthrough modulator is the compound of claim 1.

35. (canceled)

36. (canceled)

37. (canceled)

38. (canceled)

39. (canceled)

40. (canceled)

41. (canceled)

42. (canceled)

43. (canceled)

44. A method of treating cystic fibrosis, the method comprising administering (i) eRF3 modulator, (ii) an aminoglycoside, and (iii) a readthrough modulator to a subject in need thereof, wherein the readthrough modulator is the compound of claim 1.

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. The compound of claim 1, with the proviso that when R¹ and R² are both hydrogen and R⁶ is optionally substituted pyrazolyl, then R³ is not halo and R⁴ is not halo.
